(19) United States
(12) Patent Application Publication
(10) Pub. No.: US 2024/0191298 A1
(43) Pub. Date: Jun. 13, 2024

(54) DNA-BARCODED ANTIGEN MULTIMERS AND METHOD OF USE THEREOF

(71) Applicant: BOARD OF REGENTS, THE UNIVERSITY OF TEXAS SYSTEM, Austin, TX (US)

(72) Inventors: Ning JIANG, Austin, TX (US); Shuqi ZHANG, Austin, TX (US); Keyue MA, Austin, TX (US); Chenfeng HE, Austin, TX (US); Alexandra A. SCHONNESEN, Austin, TX (US)

(73) Assignee: BOARD OF REGENTS, THE UNIVERSITY OF TEXAS SYSTEM, Austin, TX (US)

(21) Appl. No.: 18/534,150

(22) Filed: Dec. 8, 2023

Related U.S. Application Data
(63) Continuation of application No. 17/046,581, filed on Oct. 9, 2020.
(60) Provisional application No. 62/655,317, filed on Apr. 10, 2018.
(60) Provisional application No. 62/719,007, filed on Aug. 16, 2018.

(51) Int. Cl. C12Q 1/6881

(52) U.S. Cl. CPC C12Q 1/6881; C12Q 2531/113; C12Q 2563/185

(57) ABSTRACT

Provided herein are methods compositions and methods to generate pMHC libraries, and methods of using the pMHC libraries to determine the sequences of T cell receptors, and T cell developmental and activation status.

### CROSS REFERENCES TO RELATED APPLICATIONS

This application is a continuation of U.S. application Ser. No. 17/046,581, filed Oct. 9, 2020, which is a US National Phase 371 application from International Application No. PCT/US2019/026757, filed Apr. 10, 2019, which claims the benefit of U.S. Provisional Application No. 62/655,317, filed Apr. 10, 2018, and U.S. Provisional Application No. 62/719,007, filed Aug. 16, 2018, all of which are hereby incorporated herein by reference in their entirety.

This invention was made with government support under Grant Nos. R00 AG040149, S10 OD020072, and R33 CA225539 awarded by the National Institutes of Health. The government has certain rights in the invention.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Dec. 1, 2023, is entitled “1414754_ST26.xml”, and is 5,768,330 bytes in size.

### BACKGROUND

### 1. Field

The present disclosure relates generally to the field of immunology. More particularly, it concerns the generation of pMHC molecules and their use in detecting T cells.

### 2. Description of Related Art

Each CD8⁺ T cell can potentially recognize multiple species of peptides bound by Major Histocompatibility Complex (pMHC) Class I molecules on the surface of most nucleated cells using a distinct TCR. This TCR-mediated reactivity and cross-reactivity affects the quality of the immune response in viral infection (Mongkolsapaya et al., 2003), auto-immune diseases (Lang et al., 2002), and cancer immunotherapy (Cameron et al., 2013). Thus, the ability to identify the antigenic peptide or peptides recognized by a T cell and its T cell receptor (TCR) sequence is essential for the monitor and treatment of immune-related diseases.

Fluorescent pMHC tetramers are widely used to identify antigen-binding T cells (Newell and Davis, 2014). While combinatorial tetramer staining can expand the number of peptides that can be interrogated, fluorescence spectral overlapping limits the number of peptides that can be examined at a time, not to mention the extent of cross-reactivity (Newell and Davis, 2014). Using isotope-labeled pMHC tetramers, mass cytometry, such as by CyTOF® (Fluidigm®), can interrogate an even larger number of peptides; however, examining cross-reactivity has not been demonstrated. Furthermore, the destructive nature of CyTOF® prohibits linking of pMHCs bound by a T cell to its TCR sequence (Newell and Davis, 2014).

DNA-barcoded pMHC multimer technology has been used for the bulk analysis of antigen-binding T cell frequencies for more than 1000 pMHCs (Bentzen et al., 2016). However, with bulk analysis, information on the binding of peptides to individual T cells is lost and cross-reactivity cannot be assessed at single cell level, which limits the assessment of cross-reactivity in primary T cells, such as T cells in clinical samples. It also remains challenging to link peptides with the individual TCR sequences that they bind to for a large number of peptides in hundreds of single T cells simultaneously. This information is valuable for tracking antigen-specific T cell lineages in disease settings, TCR-based therapeutics development (Stronen et al., 2016), and for uncovering patterns in TCR recognition (Glanville et al., 2017). One further limitation of current multimer-based methods is that while the peptide library size can be scaled up, each peptide must still be chemically synthesized for each pMHC species (Rodenko et al., 2006). The high cost associated with chemically synthesized peptides prevents the quick generation of a pMHC library that can be tailored to any pathogen or disease. Clearly, there exists a need for methods to quickly and cost effectively generate pMHC libraries to investigate T cells.

### SUMMARY

In some embodiments, the present disclosure provides compositions and methods to generate DNA barcode labeled pMHC or peptide antigen multimer libraries for hundreds or thousands of peptides, and methods of using the pMHC or peptide antigen multimer libraries to determine the following linked information at single cell level for individual T or B cells: sequences of T or B cell receptors, antigen specificity, T or B cell transcriptomic or gene expression level, and proteogenomics by the expression level of protein markers inside or on the surface of T or B cells at single cell level for individual T or B cells. This linked information is then used to assess T or B cell developmental, activation status, clonal expansion status, phenotype, antigen specificity, and funcation in different physiological or pathological conditions, such as infection, vaccination, allergy, autoimmune diseases, cancer, aging, and neurodegenerative diseases. TCR or BCR sequences and antigen sequences can be used as therapeutics in difference diseases or vaccine. The status of T or B cell developmental, activation status, clonal expansion status, phenotype, antigen specificity, and funcation can be used for immune profiling, disease early diagnosis, therapeutics development, prognosis, treatment progress monitoring, and treatment responder or non-responder separation.

In some embodiments, the present disclosure provides compositions and methods to generate pMHC libraries, and methods of using the pMHC libraries to determine the sequences of T cell receptors, and T cell developmental and activation status.

In a first embodiment, there is provided a composition comprising multimer backbone linked to a peptide-encoding oligonucleotide.

In some aspects, the multimer backbone comprises 2, 3, 4, 5, 6, 7, 8, 9, 10, or more protein subunits. In particular aspects, the multimer backbone is a dimerization antibody, engineered antibody Fab′ or similar construct that binds to a universal moiety either on a peptide or pMHC, such as the FLAG portion of the peptide or biotin, to dimerize antigens. In certain aspects, the multimer backbone is a tetramer formed by streptavidin or other similar proteins. In some aspects, the multimer backbone is a pentamer, octamer, streptamer (e.g., formed by Strep-tag), or dodecamer (e.g., formed by tetramerized streptavidin). In some aspects, the protein subunits comprise streptavidin or a glucan. In certain aspects, the glucan is dextran.

In certain aspects, the peptide-encoding oligonucleotide is further linked to a DNA handle. In some aspects, the peptide-encoding oligonucleotide is linked to the DNA handle by annealing and PCR. In some aspects, the peptide-encoding oligonucleotide is linked to the DNA handle by annealing without PCR. In some aspects, the DNA handle is an oligonucleotide comprising a first sequencing primer and a barcode. In some aspects, the barcode comprises a 8-20, such as 10-14, such as 8, 9, 10, 11, 12, 13, 14, 15, 16, 17, 18, 19, or 20, base pair degenerate sequence. In some aspects, the degenerate sequence has one or more fixed nucleotides in the middle. In particular aspects, the barcode comprises a 12 base pair degenerate sequence. In some aspects, the DNA handle further comprises a specific nucleotide sequence whose corresponding amino acid sequence can be recognized by certain proteases, such as partial FLAG (DDDDK), IEGR, or IDGR. In some aspects, the nucleotide sequence, whose amino acid sequence is recognized by proteases starts with ATG. In some aspects, the peptide-encoding oligonucleotide is further linked to a second sequencing primer.

In certain aspects, the DNA handle is linked to the multimer backbone. In some aspects, DNA barcodes denoting each type of pMHC multimer are annealed. In certain aspects, the annealing is followed by PCR. In particular aspects, each type of the pMHC multimer in the final pool has a similar DNA:multimer backbone ratio. In some aspects, the ratio of the DNA handle to multimer backbone is between 0.1:1 to 20:1, such as 0.1:1 to 1:1, 1:1 to 2:1, 2:1 to 3:1, 3:1 to 4:1, 4:1 to 5:1, 5:1 to 6:1, 6:1 to 7:1, 7:1 to 8:1, 8:1 to 9:1, 9:1 to 10:1, 10:1 to 11:1, 11:1 to 12:1, 12:1 to 13:1, 13:1 to 14:1, 14:1 to 15:1, 15:1 to 16:1, 16:1 to 17:1, 17:1 to 18:1, 18:1 to 19:1, or 19:1 to 20:1.

In some aspects, the multimer backbone is further linked to one or more detectable moieties. In particular aspects, the one or more detectable moieties comprise the barcode in the DNA handle and/or a fluorophore. In some aspects, the DNA handle or peptide-encoding oligonucleotide is linked to the detectable label. In certain aspects, the DNA handle is covalently linked to the detectable label. In particular aspects, the covalent link is a HyNic-4FB crosslink, Tetrazine-TCO crosslink, or other crosslinking chemistries. In certain aspects, the detectable moieties are attached to the multimer backbone or to the peptide-encoding oligonucleotide. In some aspects, the one or more detectable moieties are fluorophores. In some aspects, the fluorophore is a PE, PE-Cy5, PE-Cy7, APC, APC-Cy7, Qdot 565, qdot 605, Qdot 655, Qdot 705, Brilliant Violet (BV) 421, BV 605, BV 510, BV 711, BV786, PerCP, PerCP/Cy5.5, Alexa Fluor 488, Alexa Fluor 647, FITC, BV570, BV650, DyLignt 488, Dylight 649, and/or PE/Dazzle 594. In particular aspects, the fluorophores are R-phycoerythrin (PE) and allophycocyani (APC).

In certain aspects, the composition further comprises at least two peptide-major histocompatibility complex (pMHC) monomers linked to the multimer backbone. In some aspects, the composition comprises between 2 and 12, such as 2, 3, 4, 5, 6, 7, 8, 9, 10, 11, or 12, pMHC monomers.

In some aspects, the peptide-encoding oligonucleotide encodes a peptide identical to the peptide of the pMHC monomers. In some aspects, the peptide-encoding oligonucleotide comprises DNA. In certain aspects, the peptide-encoding oligonucleotide further comprises a 5′ primer region and/or a 3′ primer region.

In some aspects, the sequence of the DNA handle is constant and the sequence of the peptide-encoding oligonucleotide is variable.

In certain aspects, the pMHC monomers are biotinylated. In some aspects, the pMHC monomers are attached to the streptavidin by streptavidin-biotin interaction.

In some aspects, the composition comprises a pMHC tetramer. In other aspects, the composition comprises a pMHC pentamer.

In another embodiment, there is provided a method for generating a DNA-barcoded pMHC multimer comprising performing in vitro transcription/translation (IVTT) on a peptide-encoding oligonucleotide comprising a DNA handle, thereby obtaining the target peptide antigens; loading the peptides onto MHC monomers to produce pMHC monomers; and binding the pMHC monomers to a multimer backbone linked to a oligonucleotide comprising a DNA handle that peptide encoding oligonucleotides can use to attach or extend themselvese to the multimer backbone, thereby obtaining the DNA-barcoded pMHC multimer. In particular aspects, the DNA-barcoded multimer is a multimer of the composition of any of the above embodiments or aspects thereof. In some aspects, the MHC monomers are biotinylated. In certain aspects, the multimer backbone comprises streptavidin or streptamer. In some aspects, the multimer backbone comprises dextran. In some aspects, the DNA-barcoded fluorescent pMHC multimer is further defined as a DNA-barcoded fluorescent pMHC multimer. In some aspects, the DNA-barcoded pMHC multimer is further defined as a DNA-barcoded pMHC tetramer, pentamer, octamer, or dodecamer.

In some aspects, the method further comprises amplifying the peptide-encoding DNA oligonucleotide by PCR to add IVTT adaptors to the peptide-encoding oligonucleotide prior to performing IVTT. In some aspects, the DNA handle is an oligonucleotide comprising a first sequencing primer, a barcode, and a partial FLAG sequence. In particular aspects, the DNA handle has a constant sequence and the peptide-encoding oligonucleotide has a variable sequence. In particular aspects, the barcode comprises a 12 base pair degenerate sequence.

In some aspects, the peptide-encoding DNA oligonucleotide comprises a partial FLAG peptide at the N-terminus. In specific aspects, the partial FLAG peptide is cleaved by enterokinase after performing IVTT.

In some aspects, the peptide-encoding DNA oligonucleotide comprises a IEGR or IDGR at the N-terminus. In specific aspects, the IEGR or IDGR peptide is cleaved by factor Xa after performing IVTT.

In certain aspects, loading comprises contacting the target peptide library with MHC monomers comprising UV-cleavable temporary peptides and applying UV light to exchange the temporary peptides with the library peptides. In some aspects, loading comprises contacting the target peptide library with MHC monomers comprising non-library peptides and chemically exchanging the peptides to generate pMHC monomers. In some aspects, loading comprises unfolding the MHC monomers to release non-target peptides, contacting the unfolded MHC monomers with the target peptide library, and refolding the MHC monomers with the target peptide library to generate the pMHC monomers. In certain aspects, loading comprises contacting the MHC monomers with the target peptide library and performing CLIP peptide exchange to generate pMHC monomers. In certain aspects, loading comprises contacting the target peptide library with MHC monomers comprising temperature-sensitive temporary peptides and applying a different temperature to exchange the temporary peptides with the library peptides.

In some aspects, the DNA-barcoded pMHC or peptide multimer further comprises one or more detectable moieties. In certain aspects, the one or more detectable moieties are fluorophores. In some aspects, the fluorophores are PE, PE-Cy5, PE-Cy7, APC, APC-Cy7, Qdot 565, qdot 605, Qdot 655, Qdot 705, Brilliant Violet (BV) 421, BV 605, BV 510, BV 711, BV786, PerCP, PerCP/Cy5.5, Alexa Fluor 488, Alexa Fluor 647, FITC, BV570, BV650, DyLignt 488, Dylight 649, and/or PE/Dazzle 594. In particular aspects, the fluorophores are R-phycoerythrin (PE) and/or allophycocyani (APC).

In certain aspects, the barcoded peptide-encoding DNA oligonucleotide is generated by annealing the peptide-encoding oligonucleotide of step (a) to a linker oligonucleotide comprising a (1) region complementary to the peptide-encoding DNA oligonucleotide, (2) a barcode, and (3) a 5′ primer region and performing overlap extension. In particular aspects, the barcode is a 12 base pair degenerate sequence. In some aspects, the region complementary to the peptide-encoding DNA oligonucleotide is a partial FLAG sequence. In certain aspects, the linker oligonucleotide further comprises at least one spacer. In some aspects, the spacer is a C12 spacer and/or C18 spacer. In some aspects, the linker oligonucleotide comprises 2 spacers. In some aspects, the linker oligonucleotide further comprises an amine group. In certain aspects, the linker oligonucleotide is linked to the polymer conjugate by a covalent linkage. In particular aspects, the linker oligonucleotide is linked to the polymer conjugate by a HyNic-4FB linkage.

In another embodiment there is provided a method of generating a library of DNA-barcoded pMHC or peptide multimers comprising performing the method of any of the present embodiments by using a plurality of peptide-encoding DNA oligonucleotides. In some aspects, the peptide of each pMHC or peptide monomer is identical to a peptide encoded by the barcoded peptide-encoding DNA oligonucleotide linked to streptavidin for each DNA-barcoded pMHC multimer. In other aspects, the peptide of each pMHC or peptide monomer is different to a peptide encoded by the barcoded peptide-encoding DNA oligonucleotide linked to streptavidin for each DNA-barcoded pMHC multimer. Further provided herein is a DNA-barcoded pMHC multimer library produced by the method of the present embodiments.

In a further embodiment, there is provided a method for determining the specificity of T cell receptors (TCRs) or B cell receptor (BCR) comprising staining a plurality of T or B cells with a library of DNA-barcoded pMHC or peptide multimers of the embodiments, thereby generating pMHC multimer-bound T cells or peptide multimer-bound B cells; sorting the pMHC multimer-bound T cells or peptide multimer-bound B cells; sequencing the DNA barcode of each pMHC multimer or peptide multimer and the TCR or BCR sequences of the T or B cell bound to said pMHC multimer; and determining the copy number of each DNA-barcoded pMHC multimer bound to the corresponding T cell to determine the TCR specificity.

In another embodiment, there is provided a method for linking precursor T or B cells to their specific antigens comprising staining a plurality of T or B cells with a library of DNA-barcoded pMHC or peptide multimers of the embodiments, thereby generating pMHC multimer-bound T cells or peptide multimer-bound B cells; sorting the pMHC multimer-bound T cells or peptide multimer-bound B cells; sequencing the DNA barcode of each pMHC or peptide multimer and the TCR or BCR sequences of the T or B cell bound to said pMHC multimer; and determining the copy number of each DNA-barcoded pMHC multimer bound to the corresponding T or B cell to determine the antigen type and the TCR or BCR sequences linked to the antigen.

In some aspects of the above embodiments, the method may further comprise using the TCR sequences to determine the frequency of T cells for one or more of the target antigens in the DNA-barcoded pMHC or peptide multimer library. In some aspects, the copy number is determined by counting the number of copies of each unique barcode.

In certain aspects of the embodiments, the sorting comprises performing flow cytometry. In some aspects, flow cytometry uses a fluorophore attached to the pMHC multimer. In certain aspects, the sorting comprises separating tetramer bound T cells from unbound T cells or a sub-population of T cells. In some aspects, separating comprises using flow cytometry or using magnetically labeled antibodies or streptavidin. In certain aspects, sorting is further defined as separating each DNA-barcoded pMHC multimer-bound T cell or peptide multimer-bound B cell into a separate reaction container. In some aspects, the reaction container is a 96-well or 384-well plate. In some aspects, sorting is further defined as separating each DNA-barcoded pMHC multimer-bound T cell or peptide multimer-bound B cell in bulk. In some aspects, the cells are sorted in bulk and dispersed to the reaction container, such as a microwell plate.

In some aspects of the embodiment, the peptide-encoding oligonucleotide and DNA handle attached to the pMHC-multimer or peptide multimer form a double-stranded DNA with a 3′ polyA overhang. In some aspects of the embodiment, the peptide-encoding oligonucleotide and DNA handle attached to the pMHC-multimer or peptide multimer form a double-stranded DNA without a 3′ polyA overhang. In some aspects, sequencing comprises preparing DNA-sequencing libraries comprising at least one amplification step wherein the primer pair is used to amplify the DNA barcode of the pMHC multimer and a different primer set is used to amplify the TCRα and TCRβ sequences of each T cell. In certain aspects, a set of reverse transcription primers are used to synthesize cDNA from TCRα and TCRβ sequences of each T cell before PCR amplification. In some aspects, preparing DNA-sequencing libraries comprises nested PCR of the DNA barcodes and TCRα and TCRβ sequences of each corresponding T cell. In certain aspects, the primers used in the amplification of the DNA barcode of the pMHC multimer and the TCRα and TCRβ sequences of each corresponding T cell comprise cellular barcodes.

In certain aspects, determining TCR or BCR specificity of each T or B cell further comprises associating the TCRα and TCRβ or BCR heavy and BCR light chain sequences of the T or B cell with the count of each DNA-barcoded pMHC or peptide multimer that was bound to said T or B cell. In some aspects, the count of each DNA-barcoded pMHC multimer that was bound to said T or B cell comprises subtracting a count of irrelevant pMHC or peptide multimers bound to the T or B cell from the number of each DNA-barcoded pMHC or peptide multimers bound to the T or B cell. In certain aspects, the count of each DNA-barcoded pMHC or peptide multimer that was bound to said T or B cell comprises subtracting a count of each DNA-barcoded pMHC or peptide multimers bound to an irrelevant T or B cell clone from the count of each DNA-barcoded pMHC or peptide multimers from the T or B cell of interest. In some aspects, the count of each DNA-barcoded pMHC or peptide multimer that was bound to said T or B cell comprises subtracting a count of a DNA-barcoded MHC or peptide multimer lacking an exchanged peptide bound to the T or B cell from the count of each DNA-barcoded pMHC or peptide multimer bound to the T or B cell. In certain aspects, the count of each DNA-barcoded pMHC or peptide multimer that was bound to said T or B cell comprises generating a ratio of the MID sequences of the last suspected true binding DNA-barcoded pMHC or peptide multimer and the first suspected false binding DNA-barcoded pMHC or peptide multimer and dividing all DNA-barcoded pMHC or peptide multimers by that ratio.

In another embodiment, there is provided a method for identifying neoantigen-specific TCRs or BCRs comprising staining a plurality of T cells with a library of DNA-barcoded pMHC or peptide multimers of the embodiments, wherein the library comprises DNA-barcoded pMHC or peptide multimers, wherein the peptides in the DNA-barcoded pMHC or peptide multimer comprise a set of neoantigen peptides and/or a set of wild-type antigen peptides; sorting the T or B cells bound to the DNA-barcoded pMHC or peptide multimers; sequencing the barcodes of the DNA-barcoded pMHC or peptide multimers and the TCRs or BCRs of the corresponding T or B cell; and sorting fluorophores that are only specific to neo-antigen DNA-barcoded pMHC or peptide multimers to identify neoantigen-specific TCRs or BCRs. In some aspects, the peptide is a cancer germline antigen-derived peptide, tumor-associated antigen-derived peptides, viral peptide, microbial peptide, human self protein-derived peptide or other non-peptide T or B cell antigen.

In some aspects, the peptides in the DNA-barcoded pMHC or peptide multimers comprise a set of neoantigen peptides. In certain aspects, the peptides in the DNA-barcoded pMHC or peptide multimer comprise a set of wild-type antigen peptides. In some aspects, the peptides in the DNA-barcoded pMHC or peptide multimer comprise a set of neo-antigen peptides and a set of wild-type antigen peptides.

In some aspects, the set of neo-antigen peptides comprise a fluorophore attached to the multimer backbone and the set of wild-type antigen peptides comprise a fluorophore attached to the multimer backbone. In certain aspects, the fluorophore for the neo-antigen peptides is the same as the fluorophore for the wild-type antigen peptides. In some aspects, the fluorophore for the neo-antigen peptides is different from the fluorophore for the wild-type antigen peptides.

In some aspects, sequencing determines if the T or B cell bound only to the neo-antigen peptide, only to the wild-type antigen peptide, or to both the neo-antigen and wild-type peptides. In some aspects, if the T or B cell only bound the neo-antigen peptide, then the TCR or BCR is neoantigen-specific. In certain aspects, sorting comprises flow cytometry using fluorophore intensity of a fluorophore attached to the pMHC multimer. In some aspects, the sorting comprises separating multimer bound T cells from unbound Tor B cells or a sub-population of T or B cells. In some aspects, separating comprises using magnetically labeled antibodies or streptavidin. In some aspects, sorting is further defined as separating each DNA-barcoded pMHC or peptide multimer-bound T or B cell into a separate reaction container or in bulk. In some aspects, the reaction container is a 96-well, 384-well plate or other tubes.

In some aspects, the method further comprises repeating the steps over the course of immune therapy to monitor response to therapy. In certain aspects, the method further comprises determining a subject's immune system status and administering treatment. In some aspects, the method further comprises determining the presence of infection, monitoring immune status, and administering treatment to a subject. In some aspects, the method further comprises determining response to a vaccine. In certain aspects, the method further comprises determining the auto-antigen in an autoimmune subject and monitoring response to treatment. In some aspects, the method further comprises generating neoantigen-specific T or B cells using the identified neoantigen-specific TCRs or BCRs.

Further provided herein is a composition comprising the neoantigen-specific T cells produced by the present embodiments. Further provided is a method of treating cancer in a subject comprising administering an effective amount of the composition of the embodiments to the subject.

In another embodiment, there is provided a method for identifying antigen cross-reactivity in naïve and/or non-naïve T or B cells comprising obtaining a plurality of neoantigen- and wild type antigen-presenting of DNA-barcoded pMHC or peptide multimers of the embodiments, wherein the neoantigen-presenting DNA-barcoded pMHC or peptide multimers comprise a first fluorophore and the wild-type antigen-presenting DNA-barcoded pMHC or peptide multimers comprise a second fluorophore; staining naïve and/or non-naive T or B cells with a plurality of pMHC or peptide multimers to generate pMHC multimer-T cell complexes or peptide-multimer-B cell complexes; sorting the pMHC multimer-T cells complexes or peptide-multimer-B cell complexes; determining the TCR or BCR sequences for all sorted T or B cells; and sequencing the barcodes of the DNA-barcoded pMHC or peptide multimers and the TCRs or BCRs of the corresponding T cell which bound to the T or B cell to determine if the T or B cell only bound to the neo-antigen pMHC or peptide multimer, only the wild-type antigen pMHC or peptide multimer, or both neo-antigen and wild-type pMHC or peptide multimers, thereby identifying neo-antigens that only induce neo-antigen specific TCRs and do not induce cross-reactive TCRs or BCRs. All of these analysis can be performed on individual patients while waiting for analysis results to inform on treatment option or other medical decision as the use of IVTT allows for the quick generation of the pMHC or peptide library.

In some aspects, the first fluorophore and the second fluorophore are the same. In other aspects, the first fluorophore and the second fluorophore are different. In some aspects, the sorting is based on fluorescence intensity. In certain aspects, sorting comprises flow cytometry using fluorophore intensity of a fluorophore attached to the pMHC or peptide multimer. In some aspects, the sorting comprises separating multimer bound T or B cells from unbound T or B cells or a sub-population of T or B cells. In some aspects, separating comprises using magnetically labeled antibodies or streptavidin. In some aspects, sorting is further defined as separating each DNA-barcoded pMHC multimer-bound T cell or DNA-barcoded peptide multimer-bound B cell into a separate reaction container or in bulk. In some aspects, the reaction container is a 96-well, 384-well plate or other tubes.

In some aspects, the method further comprises repeating the steps over the course of immune therapy to monitor response to therapy. In certain aspects, the method further comprises determining a subject's immune system status and administering treatment. In some aspects, the method further comprises determining the presence of infection, monitoring immune status, and administering treatment to a subject. In some aspects, the method further comprises determining response to a vaccine. In certain aspects, the method further comprises determining the auto-antigen in an autoimmune subject and monitoring response to treatment. generating neoantigen-specific T or B cells using the identified neoantigen-specific TCRs or BCRs.

In a further embodiment, there is provided a method for preparing DNA that is complementary to a target nucleic acid molecule comprising hybridizing a first strand synthesis primer to said target nucleic acid molecule; synthesizing the first strand of the complementary DNA molecule by extension of the first strand synthesis primer using a polymerase with template switching activity; hybridizing a template switching oligonucleotide to a 3′ overhang generated by the polymerase, wherein the template switching oligonucleotide comprises a restriction endonuclease site; extending the first strand of the complementary DNA molecule using the template switching oligonucleotide as the template, thereby generating the first strand of the complementary DNA molecule which is complementary to the target nucleic acid molecule and the template switching oligonucleotide; and amplifying the complementary DNA molecule.

In some aspects, the first strand synthesis primer comprises a cellular barcode. In some aspects, the first strand synthesis primer comprises or consists of sequences in Table 1. In some aspects, the restriction endonuclease site is a SalI site. In certain aspects, the template switching oligo comprises the sequence of sequences in Table 1. In some aspects, the target nucleic acid molecule is a plurality of target nucleic acid molecules. In certain aspects, the target nucleic acid molecule is RNA, such as mRNA or total RNA. In some aspects, the polymerase with template switching activity and strand displacement is a RNA dependent DNA polymerase. In certain aspects, the polymerase is a PrimeScript reverse transcriptase, M-MuLV reverse transcriptase, SmartScribe reverse transcriptase, Maxima H Minus Reverse Transcriptase, or Superscript II reverse transcriptase. In some aspects, the target nucleic acid molecule is DNA.

In additional aspects, the method further comprises cleaving the amplified complementary DNA molecules. In some aspects, the method further comprises preparing a sequencing library from the cleaved complementary DNA molecules. In certain aspects, the further comprises adding sequencing adaptors. In some aspects, preparing a sequencing library comprises the use of a Tn5 transposase to add sequencing adaptors. In certain aspects, the sequencing adaptors comprise the sequences depicted in Table 1. In some aspects, preparing a sequencing library comprises the use of custom primers. In some aspects, the custom primers have the sequences depicted in Table 1.

Further provided herein is a method for analyzing a genome or gene expression comprising preparing a sequencing library by the method of the embodiments, and sequencing the library.

In another embodiment, there is provided a method for analyzing a gene expression from a single cell comprising providing a single cell; lysing the single cell; preparing a sequencing library by the method of the embodiments, wherein the target nucleic acid is total RNA from the single cell; and sequencing the library. In some aspects, the single cell is a human cell. In certain aspects, the single cell is an immune effector cell. In some aspects, the single cell is a T cell. In some aspects, the single cell is provided by FACS, micropipette picking, or dilution.

In yet another embodiment, there is provided a method for analyzing gene expression from a plurality of single cells comprising providing a plurality of single cells; staining the plurality of single cells with a plurality of pMHC or peptide multimers prepared by the method of the embodiments; sorting the stained single cells into individual reservoirs; lysing the single cells; concurrently preparing complementary DNA by the method of claim 117 for each of the lysed single cells; cleaving the restriction site of the complementary DNAs; pooling the cleaved complementary DNA of each of the single cells; preparing sequencing libraries from the pooled cleaved complementary DNA; and sequencing the libraries. In some aspects, the single cells are T or B cells. In certain aspects, the T or B cells are naïve T or B cells. In some aspects, the T or B cells are neoantigen binding T or B cells. In some aspects, the method further comprises performing the method of claim 89 for identifying neoantigen-specific TCRs or BCRs. In some aspects, the method is performed in high-throughput by using microdroplet methods, in-drop method, or microwell methods.

In further embodiments, there are provided additional methods in combination with any of the above embodiments. The above methods provided herein may be used to detect self-antigen specific T or B cells, wherein the self-antigen specific T or B cells cause severe adverse effect after immune checkpoint blockade therapy and other cancer immunotherapy, before a subject is administered a therapy. Also provided herein is a method of detecting T or B cell binding epitopes and further developing the T or B cell binding epitopes into vaccines or TCR or BCR redirected adoptive T or B cell therapy for any pathogens. Further, some embodiments provide a method of using common pathogen and auto-immune disease associated epitopes identified according to the present methods to test and monitor the immune health of individuals and predict individual's protective capacity to infection or likelihood of developing auto-immune diseases and monitoring the early on-set of auto-immune diseases. In addition, there is provided a method of detecting regulatory T or B cell binding epitopes according to the present methods and developing vaccines to eliminate or enhance regulator T or B cell function or number for immunological diseases.

In further embodiment, there is provided a method for analyzing T or B cell antigen specificity in combination with analyzing TCR or BCR sequences, gene expression and proteogenomics from a single cell comprising generating peptides according to the present embodiments; generating DNA-barcoded pMHC or peptide multimers of the embodiments; staining T or B cells with pMHC or peptide multimer library thereby generating pMHC or peptide multimer-bound T or B cells; sorting the pMHC multimer-bound T cells; sorting the peptide multimer-bound B cells; sequencing the DNA barcode of each pMHC or peptide multimer, the TCR TCR sequences, gene expression and proteogenomics of the T or B cell bound to said pMHC multimer; and determining the copy number of each DNA-barcoded pMHC or peptide multimer bound to the corresponding T or B cell to determine the TCR or BCR specificity.

In certain aspects, the peptide-encoding oligonucleotide is linked to the DNA handle by annealing. In some aspects, the DNA handle is an oligonucleotide comprising a first universal primer and a specific nucleotide sequence, whose corresponding amino acid sequence can be recognized by certain proteases, such as partial FLAG (DDDDK), IEGR, IDGR. In some aspects, the nucleotide sequence, whose amino acid sequence are recognized by proteases starts with ATG. In some aspects, the peptide-encoding oligonucleotide comprises a partial FLAG, IEGR or IDGR peptide at the N-terminus. In some aspects, the peptide-encoding DNA oligonucleotide is further linked to a second sequencing primer. In some aspects, the peptide-encoding oligonucleotide further comprises a polyA sequence with a length ranging from 18-30, such as 18, 19, 20, 21, 22, 23, 24, 25, 26, 27, 28, 29, or 30 base pairs. In certain aspects, the last 2-4 polyA nucleotides, such as 2, 3, or 4 nucleotides are bound by phosphothioate bonds. In certain aspects, the DNA handle is linked to the multimer backbone.

In certain aspects, the peptide-encoding oligonucleotide can be substituted with random generated oligonucleotides. Random generated oligonucleotides can comprise a partial FLAG, IEGR or IDGR peptide at the N-terminus, a random generated oligonucleotide barcode between 8-30 bp, such as 8, 9, 10, 11, 12, 13, 14, 15, 16, 17, 18, 19, 20, 21, 22, 23, 24, 25, 26, 27, 28, 29 or 30 base pairs, and a polyA sequence with a length ranging from 18-30, such as 18, 19, 20, 21, 22, 23, 24, 25, 26, 27, 28, 29, or 30 base pairs. In certain aspects, the last 2-4 polyA nucleotides, such as 2, 3, or 4 nucleotides are bound by phosphothioate bonds. In certain aspects, the DNA handle is linked to the multimer backbone.

In another embodiment, there is provided a method for the use of any of the present embodiments with single cell gene expression analysis platforms. In some aspects, the platform is the BD BD Rhapsody™ Single-Cell Analysis System, or single cell RNA sequencing (scRNA-seq) platforms, such as 10× genomics Chromium, 1CellBio inDrop or Dolomite Bio Nadia. In some aspects, the method is combined with DNA-labeled antibody sequencing, such as CITE-seq or REAP-seq or commercially available DNA-labeled antibodies, such as BD Ab-seq products or Biolegend TotalSeq.

The present method including the TetTCR-Seq, single cell gene expression or scRNA-seq, and DNA-labeled antibody sequencing is referred to herein as TetTCR-SeqHD. TetTCR-SeqHD can use peptide or antigen encoding oligonucleotides with poly A tail or random oligonucleotides with poly A tail barcoding antigen speicficity added to the 3′end to interface with scRNA-seq protocols that high-throughput scRNA-seq platforms use. In some aspects, the DNA linker oligonucleotide or DNA handle is covalentely linked to streptavidin in order to complementary bind peptide-encoding DNA oligonucleotide or random oligonucleotide barcoding antigen speicficity. In some aspects, the method only comprises annealing to link the peptide-encoding DNA oligonucleotide to the streptavidin. MID or UMI and cell barcodes from high-throught platforms during reverse transcription may be used. Reverse transcription using primers containing polyT in the above single cell analysis platforms can generate cDNA of peptide-encoding DNA oligonucleotide for each individual cell.

In some aspects, the proteinase is not limited to enterokinase, enteropeptidase or factor Xa. Any enzyme with a specific cleavage site and the peptides encoding the cleavage site can be used here to construct the DNA handle or liner sequences and paired with that enzyme in generating peptides.

In particular aspects, the reverse transcription part of TetTCR-SeqHD is compatible with single cell RNA sequencing protocols, such as Smart-seq and Smart-seq2 protocols. In certain aspects, amplification of the peptide or antigen encoding oligos with poly A tail or random oligonucleotide with poly A tail barcoding antigen specificity is accomplished using the single cell gene expression analysis platforms or single cell RNA sequencing protocols, such as Smart-seq and Smart-seq2 protocols or by adding a primer that anneals to the 5′ end of the peptide or antigen encoding oligos with poly A tail or random oligonucleotide with poly A tail barcoding antigen specificity.

Further provided herein is a method to generate a set of peptides using oligonucleotides that encode the peptides but without a polyA tail by using a separate set of random barcoded oligonucleotides with a long poly A tail to covalently attach to a multimer backbone via a DNA linker or handle. The random barcoded oligonucleotides with poly A tail can be used in the reverse transcription. This set of random barcoded oligonucleotides with poly A tail can be re-used between cohort of samples or patients while only changing the short oligonucleotides that encode peptide to match specific antigens one wants to test in the sample or neo-antigens identified in individual patients.

In some aspects of any of the above embodiments, the methods comprise reading of the antigen specificity by qPCR without performing sequencing. This method can be applied to a set of pre-defined oligonucleotides that are used to denote peptide antigens.

In a further embodiment, there is provided a method comprising reading antigen specificity by qPCR without performing sequencing in combination the with above embodiments.

In another embodiment, there is provided a method to determine whether predicted cancer antigens or foreign antigens or self-antigens are presented by MHC on cancer cells or virally infected host cells or host cells comprising generating a pMHC multimer library by according to the embodiments; using the pMHC multimer library to identify polyclonal T cells from patients or healthy individuals to culture; expanding polyclonal T cell culture and exposing the T cells to either cancer cells, virally infected cells or host cells to be activated by antigens presented by their MHC molecules; and performing TetTCR-Seq or TetTCR-SeqHD to examine the antigen specificity and activation status at single T cell level to determine which antigen-recognizing T cells have been activated, which indicates the existence of that antigen or antigens on the surface of target cells that T cells were exposed to.

In a further embodiment, there is provided a method of identifying linked antigen targets and recognizing B cell receptors or antibodies according to the embodiments.

Further provided herein is a method of detecting self-antigen specific T or B cells according to the embodiments, wherein the self-antigen specific T or B cells cause severe adverse effect after immune checkpoint blockade therapy in a disease, preventive vaccine or therapeutic vaccine.

In another embodiment, there is provided a method of detecting T or B cell binding epitopes according to the embodiments and developing the T or B cell binding epitopes into vaccines or TCR or B cell receptor redirected adoptive T or B cell therapy or antibody-based therapies in a disease, preventive vaccine or therapeutic vaccine.

A further embodiment provides a method of using pathogen and autoimmune disease-associated protein epitopes identified according to the embodiments to monitor the immune health of a subject by associated T or B cell number changes or associated gene signature of T or B cells in a disease, preventive vaccine or therapeutic vaccine.

A method of detecting regulatory T or B cell binding epitopes according to any one of claims 1-178 and developing vaccines to eliminate or enhance regulator T or B cell function or number for a disease or preventive vaccine or therapeutic vaccine.

In any of the above embodiments, the disease or preventive vaccine or therapeutic vaccine is in cancer, an infectious disease, autoimmune disease, autoimmune disease, neurodegenerative disease, allergy, asthma, organ transplantation, bone marrow transplantation, trauma, wound, psychological diseases, cardiovascular diseases, diseases of the endocrine system, diseases of any organ or tissue or cells of the human body, or aging.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1I: Workflow for generation of DNA-BC pMHC tetramer library and proof-of-concept of using TetTCR-Seq for high-throughput linking of antigen binding to TCR sequences for single T cells. (a) Workflow for generation of DNA-BC pMHC tetramers. Grey text boxes denote step order and names. (b) DNA-BC pMHC tetramer libraries are used to stain and isolate rare antigen-binding T cell populations from primary human CD8⁺ T cells by magnetic enrichment. Cells are single-cell sorted into lysis buffer and RT-PCR is performed to amplify both the TCRαβ genes and the DNA-BC to determine the pMHC specificities by NGS. Shown is Experiment 1, a proof-of-concept, using a 96 peptide library to link antigenic peptide binding to TCR sequences for hundreds of single T cells. (c) CMV-NLV peptide generated from either IVTT or conventional synthetic (Syn) method were used to form pMHC tetramers in order to stain either a cognate or a non-cognate T cell clone. (d) MID counts per peptide detected on single T cells sorted from the Tetramer fraction in Experiment 1 (16 out of 768 peptides, aggregated from 8 cells, had >0 MID counts). Dashed line represents MID threshold for identifying positively bound peptides. (e) Peptide rank curve by MID counts for each of top 10 ranked peptides in the order of high-to-low for single sorted cells from the spike-in clone (8 cells) in Experiment 1. Black dashed line represents MID threshold for identifying positively bound peptides as defined in (d). Each solid line represents the MID counts for each of the 96 peptides that can potentially bind on a single cell with only top 10 peptides, by MID counts, are shown. Blue solid lines indicate cells with at least one positively binding peptide; Inset pie charts indicate proportion of cells with the indicated number of positively binding peptides. (f) Fluorescent intensity of the HCV-KLV(WT) binding T cell clone, used as spike-in in Experiment 1, stained individually with the indicated pMHC tetramers, generated using Syn peptides, in a separate validation experiment. (g) Peptide rank curve by MID counts as in (e) for the Tetramer⁺ primary T cell populations (167 cells) in Experiment 1. Black dashed line and blue solid lines are similarly defined as in (e). Grey solid lines indicate cells that did not positively bind any peptides based on the criteria discussed at the beginning of the Supplementary Information. (h) Calculated frequencies of antigen-binding T cell populations in total CD8⁺ T cells for peptide antigens with at least 1 detected T cell, separated by phenotype. (i) V-gene usage of unique TCR sequences that are specific for YFV_LLW (naïve and non-naïve combined, n=11 for TRAV, n=15 for TRBV) or MART1 A2L (naïve and non-naïve combined, n=33 for TRAV, n=43 for TRBV). Fl, fluorescence intensity. MFI, Median Fluorescence Intensity. a.u., arbitrary unit. APL, altered peptide ligand.

FIGS. 2A-2H: High prevalence of neo-antigen binding T cells that cross-react to WT counterpart peptides and high-throughput isolation of neo-antigen-specific TCRs for multiple specificities in parallel using TetTCR-seq. (a-c) Experiment 3, isolation of single Neo and/or WT binding T cells from a healthy donor using a 40 Neo-WT antigen library. (a) DNA-BC pMHC tetramer staining profile of naïve CD8⁺ T cells from the tetramer pool-enriched fraction. (b) Relative proportion of T cells among the three possible antigen binding combinations (Neo⁺WT⁻, Neo⁻WT⁺, Neo⁺WT⁺) for each Neo-WT antigen pair from Experiment 3. Data was filtered to only include pairs where both peptides were or detected in at least one cell, and have at least 3 detected cells total (149 cells, see Methods). (c) Neo-antigens in (b) were grouped based on mutation positions, middle (4-6) or fringe (1-3, 7-9). Statistical test was performed between the two groups on associated percentage of cross-reactive T cells as red bars shown in (b). Each circle denotes one Neo-WT antigen pair (n=11, One-tailed Mann Whitney U-Test). (d-f) Experiment 5 and 6, isolation of Neo and/or WT binding T cells using a 315 Neo-WT antigen library. (d) DNA-BC pMHC tetramer staining profile of naïve CD8⁺ T cells from the tetramer pool-enriched fraction for Experiment 5. See Supplementary FIG. 15 for gating scheme. (e) Percent cross-reactive T cells for Neo-WT antigen pairs based on the mutation position of the neo-antigen. Same data filter as (b) is used. Each circle denotes one Neo-WT pair (n=517 cells, see Supplementary Information). (f) Neo-antigens in (e) were grouped based on mutation position (left) or PAM1 value (right). Red bars denote median. Statistical test was performed between the two groups as indicated on associated percentage of cross-reactive T cells as shown in (e). (n=62, One-Tailed Mann Whitney U-Test). (g) LDH cytotoxicity assay on in vitro expanded primary T cell lines sorted using DNA-BC pMHC tetramers as in (a) interacting with T2 cells pulsed with the 20 neo-antigen peptide pool or 20 WT counterpart peptide pool. Each pair of black/grey bars represent one T cell line derived from sorting 5 cells from one of the three indicated populations in (a). Each condition was performed in triplicates. Standard deviation is shown for each condition. (h) Fluorescent intensity histogram of Jurkat 76 cell line transduced with TCRs from Experiment 3 and 4 stained with indicated tetramers. One TCR, AB5, was identified to only recognize the neo-antigen, GANAB_S5F, while the other TCR, M11, was identified to be cross-reactive to both the neo-antigen, GANAB_S5F and its WT counterpart, GANAB, from TetTCR-Seq. Fl, fluorescence Intensity. a.u., arbitrary unit.

FIGS. 3A-3E: pMHC tetramers produced by IVTT has similar staining performance as the conventional method using chemically synthesized peptide. (a-e) pMHC tetramers, containing the indicated peptide, were generated using IVTT or chemically synthesized and used to stain a cognate and non-cognate T cell clone. Anti-CD8a (RPA-T8) was present throughout the staining.

FIGS. 4A-4F: IVTT can generate 20-100 μM of the desired peptide. (a-f) Peptides generated from either IVTT or the traditional, synthetic peptide method were diluted at different ratios and were used to form PE labeled pMHC tetramers. Starting concentration of synthetic peptide is 100 μM for all peptides. These pMHC tetramers were used to stain a cognate T cell clone. Anti-CD8a (RPA-T8) was present throughout the staining. MFI: Median Fluorescence Intensity. a.u.: arbitrary unit.

FIGS. 5A-5D: Covalent attachment of DNA-BC to PE and APC streptavidin does not affect staining intensity of the resulting tetramers. (a-d) PE and APC labeled streptavidin were covalently attached with DNA linker at a molar ratio of 3-7 streptavidin molecules per one molecule of DNA-BC. An oligonucleotide encoding HCV-KLV(WT) was annealed to streptavidin-conjugated DNA linker and extended to form DNA-BC. DNA-BC pMHC tetramers were formed with either the HCV-KLV(WT) or TYR-YMD peptide and with either PE or APC streptavidin scaffold, as indicated. Resulting tetramers were used to stain a cognate and non-cognate T cell clone. Anti-CD8a (RPA-T8) was present throughout the staining. Fl: fluorescence intensity. a.u.: arbitrary unit.

FIGS. 6A-6E: Quantification of the detection limit of DNA-BC pMHC tetramers. (a) Fluorescence of PE-Quantibrite™ beads that were used for (b) calibration of PE fluorescence intensity to protein abundance. (c) PE labeled, DNA-BC pMHC tetramers containing the HCV-KLV(WT) peptide (with the DNA-BC corresponding to HCV-KLV(WT) sequence) was used to stain a cognate T cell clone at the indicated tetramers dilutions starting at 5 μg/ml for 1×. Anti-CD8a (RPA-T8) was present throughout the staining. (d) Calculation of tetramer abundance on each of the staining dilutions from (c) using the calibration curve from (b). Corrected value indicates subtraction of background value from the unstained cell population. (e) qPCR of DNA-BC on single cells sorted from various populations. Tet Dilution 1×-625× are the 5 tetramer dilutions from (c), amplified with primers specific for DNA-BC encoding the HCV-KLV(WT) sequence. Negative control #1 is a GP100-IMD binding T cell clone that has been stained with 1× dilution of the DNA-BC HCV-KLV(WT) tetramer as in (c), amplified with primers specific for DNA-BC encoding the HCV-KLV(WT) sequence. Negative control #2 is two PE labeled DNA-BC pMHC tetramer were made containing the HCV-KLV(WT) or GP100-IMD peptide. Each tetramer contains a DNA-BC sequence that corresponds to the peptide. The two tetramers were pooled and used to stain the HCV-KLV(WT) binding clone in (c) at 5 μg/ml each (none diluted). qPCR was performed using primers specific for DNA-BC encoding GP100-IMD only (which corresponds to bound GP100-IMD tetramer). Each circle indicates a qPCR reaction with one sorted cell. 0 Cq value represents no detected amplification after 40 cycles. Red bars indicate the mean Cq value for positively amplified cells.

FIGS. 7A-7D: Gating scheme and sorting strategy for Experiment 1 and 2. (a) Representative gating scheme for Experiment 1 and 2. Shown is gating scheme for Experiment 1. Single-cell lymphocytes were first gated. The HCV-specific T cell clone spike-in, pre-stained with BV605-CD8a, and the primary T cell population, stained with BV785-CD8a, were isolated. CD8⁺ T cells were gated to be 7-AAD⁻CD3⁺. Naïve and non-naïve antigen-binding cells were sorted from the PE⁺, endogenous peptides and APC⁺, foreign peptides. The same antibody panel and gating scheme is used for Experiment 2. (b) Tetramer staining of flow-through fraction was used to set the PE and APC tetramer negative and positive gates. An example from Experiment 1 was shown. (c) Frequency of the four antigen-binding T cell populations for Experiment 1 and 2. (d) Percent of naïve cells from Foreign and Endogenous Tetramer⁺ CD8+ T cells for Experiment 1 and 2. Bulk indicates flow-through CD8+ T cells from the same experiment. (d) Frequency of the four antigen-binding T cell populations for Experiment 1 and 2.

FIGS. 8A-8E: Processing of DNA-BC sequencing reads for sort 1. Reads within the same cell barcode that have the same MID sequence were clustered together and were considered as one MID. A consensus peptide-encoding sequence was generated for each cluster. (a) MIDs were filtered to only include those having the peptide-encoding sequence be a length of 25-30. All peptides used were 9-10 AA in length, so the DNA length should be 27 and 30. (b) MIDs were then filtered such that the closest Levenshtein distance of the peptide-encoding sequence to the reference DNA-BC list is no greater than 2. (c) Percent of total reads belonging to each group of MIDs sharing the same read count. MIDs with low read counts (left of the vertical dashed line) were discarded as sequencing error. The resulting MIDs can then be assigned to each sorted T cell according to the cell barcode. (d, e) Total MID counts associated with each cell from the PE⁺ (d) and APC⁺ (e) populations from experiment 1 were compared to their corresponding tetramer staining intensity from index sorting analysis. Each circle denotes one cell. Line indicates linear regression and the associated R-squared value.

FIGS. 9A-9F: Verification of pMHC classification using the spike-in HCV-KLV(WT) binding clone and primary cells with shared TCRs for experiment 1. (a) Top 10 pMHC specificities of the sorted spike-in HCV-KLV(WT) binding clone, ordered by MID count from high-to-low. Bold border separates detected and non-detected binding peptides by the criteria. (b) In a separate experiment, T cell clone from (a) was stained with the indicated conventional pMHC tetramers in separate tubes in the presence of anti-CD8a (RPA-T8). (c,d) Bolded peptides outside the true binding peptide threshold in (a) were tested for pMHC tetramer staining as in (b). (e) MID count for the top 8 ranked peptides for the tetramer+ primary T cells with shared TCRα and/or TCRβ sequence. Dashed line indicates MID count threshold for identifying positive binding peptides. (f) Top 5 peptides by MID count for T cells sharing at least one TCRα or β chain from (e). Bold border separates positive and non-specific binding peptides (SEQ ID NOs: 1621, 1592, 1618, 1614, 1616, 1711, 1705, 1712 and 1719, respectively, left to right by column, top to bottom by row, respectively first appearance only).

FIGS. 10A-10D: Analysis of Experiment 2. (a) MID counts greater than 0 from peptides in the Tetramer population (n=8 cells). (b) Peptide rank curve by MID counts for all primary T cells. Dashed lines indicate MID threshold for identifying positively bound peptides. Each solid line indicates a cell and only the top 8 peptides were shown ranked by their MID counts. Blue solid lines indicate cells with at least one positively binding peptide; grey solid lines indicate cells that did not positively bind any peptides based on the criteria discussed at the beginning of the supplementary information. Insert pie chart indicate proportion of cells with the indicated number of positively bound peptides. In the insert, paired indicates detection of 2 antigens; one for a wildtype antigen and one for an altered peptide ligand with one amino acid substitution. This was found for GP100 and NY-ESO-1 (Supplementary Table) (c) V-gene usage of TCR sequences that are specific for YFV_LLW (n=27 for TRAV, n=29 for TRBV) or MART1_A2L (n=37 for TRAV, n=39 for TRBV). Only distinct TCR sequences were used (one clonal population counts for only one TRAV and/or one TRBV). (d) Estimated frequencies of antigen-binding T cell populations in total CD8⁺ T cells with at least 1 detected cell, separated by phenotype. It was found that CMV and EBV-specific T cells accounted for the majority of this donor's non-naïve repertoire, which corroborates the CMV and EBV seropositive status of this individual. In agreement with Experiment 1, it was found that, among peptides surveyed, naïve T cells contained greater diversity of antigen specific T cell populations compared to the non-naïve compartment, which is highly skewed towards a select few antigen specific T cell populations. It was also found the same dominance in TCRα V gene usage among the MART1-A2L and YFV-LLW specific TCRs in this donor compared to Experiment 1.

FIGS. 11A-11D: Gating scheme and sorting strategy for Experiment 3 and 4. (a) Representative gating and sorting scheme for Experiment 3 and 4. Gating scheme for Experiment 3 is shown. (b) Tetramer gating on the flow-through fraction of Experiment 3 (c) Estimated frequency of the sorted Tetramer⁺ populations for Experiment 3 and 4. (d) Percentage of naive cells of the indicated Tetramer⁺ CD8+ T cell population of total Tetramer⁺ T cells for Experiment 3 and 4. Bulk refers to the flow-through from the same experiment.

FIGS. 12A-12E: Analysis for Experiment 3. (a) MID counts for each peptide from each cell from the Tetramer population (12 cells, 42 peptides each). (b-d) Peptide rank curve by MID counts for the top 5 peptides for Neo⁺WT⁻ (b), Neo⁻WT⁺ (c), and Neo⁺WT⁺ population (d) for Experiment. Dashed lines indicate MID threshold for identifying positively bound peptides. Each solid line indicates a cell and only the top 5 peptides were shown raked by their MID counts. Blue solid lines indicate cells with at least one positively binding peptide; grey solid lines indicate cells that did not positively bind any peptides based on the criteria discussed at the beginning of the supplementary information. Insert pie charts for all three panels indicate proportion of cells with the indicated number of positively bound peptides. (e) Cell count for all detected peptides for each Neo-WT antigen pair (n=223 cells) (g) Number of Neo⁺WT⁻, Neo⁻WT⁺, and Neo⁺WT⁺ peptides that are targeted by TCRs with successfully recovered TCRαβ sequences.

FIGS. 13A-13C: Verification of pMHC classification using the spike-in HCV-KLV(WT) binding clone and primary cells with shared TCRs in Experiment 3. (a) Top 5 epitopes by MID count for T cells sharing at least one TCRα or β chain. Bold border indicates the positively-classified binding peptides. TCRα or β chains with the same color in the same cluster have the same nucleotide sequence for the respective chain. (b,c) Peptide rank curve by MID counts for the HCV-KLV(WT) binding spike-in clone (12 cells) (SEQ ID NOs: 1776, 2204, 2199, 2236, 2164, 2261, 2300, 2306, 2505, 2506, 2425, 2445, 2482, 2487 and 2447, respectively, left to right by column, top to bottom by row, first appearance only) (b) and primary cells with shared TCR (13 cells) (c). Dashed lines indicate MID threshold for identifying positively bound peptides. Each solid blue line indicates a cell and only the top 5 peptides were shown raked by their MID counts. For (c) only cells with identical TCRα and TCRβ sequence on an AA level were considered, corresponding to cluster 1a, 2, 5, and 6 in (a). For WT-antigen, the peptide was named after the protein; for Neo-antigen, the peptide was named as protein name_AA #AA.

FIGS. 14A-14H: DNA-BC analysis for Experiment 4. (a) MID counts associated with peptides from the sorted Tetramer⁻ CD8⁺ T cells (36 cells). MID threshold for positively binding peptide is designated by the dashed line. (b-d) Peptide rank curve by MID counts for the (b) Neo⁺WT⁻, (c) Neo⁻WT⁺ and (d) Neo⁺WT⁺ primary cells. Dashed line indicates MID threshold for identifying positively bound peptides. Each solid line indicates a cell and only the top 5 peptides were shown ranked by their MID counts. Blue solid lines indicate cells with at least one positively binding peptide; grey solid lines indicate cells that did not positively bind any peptides based on the criteria discussed at the beginning of the supplementary information. Insert pie charts for all three panels indicate proportion of cells with the indicated number of positively bound peptides. (e) Cell count for all detected peptides for each Neo-WT gene pair (n=274 cells). (f) Relative proportion of the three cell populations for each Neo-WT gene pair from (e), similar to FIG. 2B. Each antigen was normalized by the relative frequency and number of cells sorted from the corresponding Tetramer⁺ population (see Methods). Only pairs where both the Neo-antigen and Wildtype were detected in at least one cell, and have at least 3 detected cells total were considered (n=200 cells). (g) Comparison of cross-reactivity for Neo-WT antigen-binding T cell populations from (f) that have mutations near the middle or fringes (n=11 Neo-WT antigen pairs, One-tailed Mann-Whitney U Test). (h) Comparison of the percent cross-reactive T cells that exist within each Neo-WT antigen-binding T cell population between Experiment 3 and 4. Only Neo-WT pairs that meet the criteria in (f) and are shared between the two experiments are considered. Dot represents one Neo-WT pair and lines connect the same pair from the two experiments (n=18, One-tailed Wilcoxon Signed-Rank Test).

FIGS. 15A-15E: Validation for “undetected” peptides in Experiment 3 and 4. (a) ELISA for all 40 pMHC monomers UV-exchanged with IVTT-generated Neo or WT peptides. UV-exchanged pMHC monomers are plated at a concentration of 1.6 nM estimated based on the un-exchanged MHC monomer concentration, followed by anti-β2M staining. Blue dots represent un-exchanged MHC monomer diluted at various concentration from lowest to highest (0.05, 0.25, 1.25, 6.25, 31.25 nM). Red dot represents UV-exchanged pMHC in IVTT solution that did not contain a peptide-encoding DNA template. Black dots indicate the 5 “undetected” peptides in Experiment 3 and 4. Solid line is a sigmoidal model fit to the standards. Arrows indicate “undetected” peptides from Experiment 3 and 4. (b) TetTCR-Seq experiment on an additional donor's PBMC sample using an IVTT-generated pMHC tetramer library for PPI_ALWM and the five “undetected” peptides. Shown is the estimated frequency of each antigen-binding CD8+ T cell population. (c-e) Peptide titration experiments were performed for three of the “undetected” peptides where T cell clones could be generated using Tetramer⁺ T cells from (b). Peptides generated from either IVTT or the traditional, synthetic peptide method, were diluted at different ratios and were used to form PE labeled pMHC tetramers. Starting concentration of synthetic peptide is 100 μM for all peptides. These pMHC tetramers were used to stain a cognate T cell clone. Anti-CD8a (RPA-T8) was present throughout the staining. MFI, Median Fluorescence Intensity. a.u., arbitrary unit. For WT-antigen, the peptide was named after the protein; for neo-antigen, the peptide was named as protein name_AA #AA.

FIGS. 16A-16D: Gating scheme and sorting strategy for Experiment 5 and 6. (a) Representative gating scheme for Experiment 5 and 6. Shown is the gating scheme for Experiment 5. (b) Tetramer gating on the flow-through fraction from Experiment 5. (c) Estimated frequencies of the three Tetramer⁺ populations for Experiment 5. Frequencies could not be obtained for Experiment 6. (d) Naïve T cell percentages for each of the three Tetramer⁺ populations and bulk flow-through CD8+ T cells for Experiment 5 and 6.

FIGS. 17A-17K: Analysis of Experiment 5 and 6. (a-h) MID counts associated with peptides from the sorted Tetramer⁻ CD8⁺ T cells for Experiment 5 (a) and 6 (e). Peptide rank curve by MID counts for the indicated Tetramer⁺ cell populations for Experiment 5 (b-d) and 6 (f-h). Dashed line indicates MID threshold for identifying positively bound peptides. Each solid line indicates a cell and only the top 8 peptides were shown ranked by their MID counts. Blue solid lines indicate cells with at least one positively binding peptide; grey solid lines indicate cells that did not positively bind any peptides based on the criteria discussed at the beginning of the Supplementary Information. Insert pie charts for all these panels indicate proportion of cells with the indicated number of positively bound peptides. For insert pie charts, 2+ Paired indicates that all detected peptides from a given cell belong to a particular Neo/WT antigen pair; this has the same meaning as “2” in pie chart inserts of Experiment 3 and 4, but since one WT was included that had two neo-antigens in this library (DHX33-LLA) it was found one cell that was cross reactive to all three peptides, which is counted in this category as well. 2+ unpaired indicates at least 2 detected peptides but at least one peptide did not belong to a particular Neo/WT antigen pair. (i) Total cell counts for Neo-WT antigen pairs with at least one detected cell (n=678 cells). (j) As in FIG. 2f, a greater difference in the percent of cross-reactive antigen-binding populations is observed when revising the peptide middle position to position 3-7. Each circle represents the percent of cross-reactive T cells observed for one Neo-WT antigen pair. Only antigen pairs where both the Neo and WT peptides were detected in at least one cell, with at least 3 cells total are included. Bars denote median. (n=62 Neo-WT antigen pairs, One-tailed Mann-Whitney U Test). (k) Definition of PAM1 high/low threshold. PAM1 values for amino acid pairs i and j are calculated by adding the one directional PAM1 values, PAM1ᵢⱼ+PAM1ⱼᵢ, as defined by Wilbur et al. Shown is a histogram of all the possible PAM1 values between non-identical amino acids (n=190 AA transitions). The top 10% is designated as PAM1 High.

FIG. 18: ELISA on the 315 pMHC monomer library UV-exchanged with IVTT-generated peptides for Experiment 5 and 6. UV-exchanged pMHC monomer using IVTT-generated peptides are plated on ELISA plates at a concentration of 1.6 nM estimated from unexchanged MHC monomer concentration and then stained with anti-β2m antibody. Blue circles represent pMHC concentration standards. Solid line represents sigmoidal model fit to the standards. Red dot represents UV-exchanged pMHC in IVTT solution that did not contain a peptide-encoding DNA template, thus serves as a negative control. Black dots represent peptides that were not detected in Experiments 5 or 6. Green diamonds represents peptides that were detected in at least one cell in Experiment 5 or 6. Top histogram combines both the detected and undetected peptides in respect to pMHC monomer concentration plotted below. Dashed line represents the minimum threshold for pMHC UV-exchange. The blue dot standard to the right side of the dashed line is 0.4 nM of un-exchanged MHC monomer.

FIG. 19: Both PE and APC fluorescent DNA-BC pMHC tetramers can be used to sort neo-antigen-specific T cells with no functional reactivity to WT counterpart peptide. A DNA-BC pMHC library was constructed as in Experiment 3 and 4 to sort APC⁺PE⁻ (Neo⁺WT⁻) primary T cells. A fluorescence swapped pMHC library compared to Experiment 3 and 4, where neo-antigen pMHCs were on the PE channel and WT pMHCs were on the APC channel, was used to sort PE⁺APC⁻ (Neo⁺WT⁻) primary T cells. 5 cells were sorted per well for in vitro culture. LDH cytotoxicity assay on in vitro expanded primary T cells sorted interacting with T2 cells pulsed with the 20 neo-antigen peptide pool or 20 WT counterpart peptide pool. Each pair of black/grey bars represent one T cell line. Each condition was performed in triplicates. Standard deviation is shown for each condition.

FIGS. 20A-20C: Characterization of the Neo⁺WT⁻ and Neo⁺WT⁺ cell lines in FIG. 2G. (a,b) T cell clonal composition as assessed by single cell TCR sequencing and matched pMHC specificity for the T cell lines in the Neo⁺WT⁻ (a) and Neo⁺WT⁺ (b) of FIG. 2g. For (a), TetTCR-Seq was performed for pooled cell lines and the resulting single sorted cells were matched to the correct T cell line from bulk TCR sequencing results of each T cell line (SEQ ID NOs: 4406-4425, respectively, left to right by column, top to bottom by row). For (b), TetTCR-Seq was performed on each T cell line using the 40 Neo-WT DNA-BC pMHC tetramer library. Single cell DNA-BC and TCR sequences were used to tally the T cell clonality and the antigen binding of each T clone within a T cell line. For WT-antigen, the peptide was named after the protein; for neo-antigen, the peptide was named as protein name_AA #AA (SEQ ID NOs: 4406-4425, respectively, left to right by column, top to bottom by row). (c) LDH cytotoxicity assay on the monoclonal T cell Neo⁺WT⁺ lines, discovered from (b), using the pMHC identified by TetTCR-Seq. Each condition performed in triplicates. “Neo pool-1” and “WT Pool-1” refers to the other 19 Neo-antigens and Wildtype peptides, respectively, that were not identified by TetTCR-Seq for the given cell line. HCV-KLV peptide was used as a known-antigen negative control.

FIGS. 21A-21B: Tetramer staining of additional Jurkat 76 cell lines transduced with TCRs identified from Experiment 3. Jurkat 76 cells were transduced with the indicated TCRs, derived from primary T cell with positively identified antigens from Experiment 3, and then stained with the indicated pMHC tetramers. (a) A pair of TCRs that were identified to be cross reactive for both the Neo-antigen and Wildtype versions of SEC24A or just the Wildtype from TetTCR-Seq. (b) a TCR identified to be cross reactive for the Neo-antigen and Wildtype versions of NSDHL from TetTCR-Seq. Fl, fluorescence Intensity. a.u., arbitrary unit. For WT-antigen, the peptide was named after the protein; for Neo-antigen, the peptide was named as protein name_AA #AA.

FIGS. 22A-22D: 3′ end sequencing for highly multiplexed single cell RNA-seq (3′end scRNA-seq) is robust and reproducible. (a) Illustration of workflow of 3′end scRNA-seq. (b) Comparison of ERCC detection efficiency between 3′end scRNA-seq and published scRNA-seq data using Fluidigm C1. (c) 3′end scRNA-seq is robust in gene expression quantification compared to original Smart-seq2. (d) 3′end scRNA-seq has very low cross-contamination rate.

FIGS. 23A-23B: Schematics of TetTCR-SeqHD. (a) Workflow of generating DNA-labeled tetramer for TetTCR-SeqHD. (b) Workflow of application of TetTCR-SeqHD to study gene expression, phenotype, and TCR repertoire of antigen specific T cells

FIGS. 24A-24D: TetTCR-SeqHD of CD8+ T cell clones. (a) The different antigen specific T cell clones used and the types of TCRβ among these polyclonal populations. (b) The distribution of TCRβ species within each polyclonal population. (c) Sequencing metrics of TetTCR-SeqHD on T cell clones. (d) Density plot of MID counts (log 10) of self and foreign peptides.

FIGS. 25A-25C: Data quality metrics for T cell clones. (a) Histogram of predicted antigen specificity using pMHC DNA barcodes. Within each predicted antigen specificity, the stacked bar denotes distribution of the true antigen specificity based on TCRβ sequence. (b) The recall and precision rate of antigen specificity identification using pMHC DNA barcodes. (c) Table showing the recall, precision and false discovery rate of antigen specificity identification using pMHC DNA barcodes for each clone.

FIG. 26: Circos plot showing the distribution of TCRβ species within each predicted antigen specificity using pMHC DNA barcodes.

FIGS. 27A-27F: TetTCR-SeqHD of enriched CD8+ T cells from frozen healthy blood donors' PBMCs. (a) Density plot of MID counts (log 10) of self and foreign peptides. (b) Histogram of MID counts (log 10) of self and foreign peptides. Dashed line is the negative threshold to call positive tetramer binding events. (c) tSNE analysis of single cell gene expression. Red dots are foreign-antigen specific cells and blue dots are self-antigen specific cells. The antigen specificities were predicted by pMHC DNA barcodes. (d) PCA analysis of antigen specific gene expression characters. (e) Heatmap showing the predicted antigen specificities for the top 10 abundant TCRs with unique TCRα and TCRβ. (f) Table showing the percentage of foreign antigen, self-antigen and negatives in each donor, as well as the ratio between number of foreign and self-antigen specific cells predicted using pMHC DNA barcodes in comparison with flow cytometry. Donor849_negative is the sorted tetramer negative population.

FIG. 28: AbSeq of antigen specific CD8⁺ T cells. Left: tSNE and phenograph clustering analysis using gene expression and antibody expression. Right: Antibody expression of CD45RA, CD45RO, CD197 and CD95.

### DESCRIPTION OF ILLUSTRATIVE EMBODIMENTS

It has been a challenge to link peptides with the individual TCR sequences that they bind, compounded when analyzing a large number of peptides in hundreds of single T cells simultaneously. The addition of molecular identifiers to TCR sequencing can improve the accuracy of TCR sequencing. Further, by probing a large number of T cells with MHCs that have been modified to house specific peptides, TCR sequences can be associated with the antigens that they bind. Accordingly, in certain embodiments, the present disclosure provides methods to use molecular identifiers to increase sequencing accuracy and peptide MHC tetramers to stain T cells, in order to link TCR sequences to their antigen.

In some embodiments, the present disclosure provides compositions and methods to generate DNA barcode labeled pMHC or peptide antigen multimer libraries for hundreds or thousands of peptides, and methods of using the pMHC or peptide antigen multimer libraries to determine the following linked information at single cell level for individual T or B cells: sequences of T or B cell receptors, antigen specificity, T or B cell transcriptomic or gene expression level, and proteogenomics by the expression level of protein markers inside or on the surface of T or B cells at single cell level for individual T or B cells. This linked information is then used to assess T or B cell developmental, activation status, clonal expansion status, phenotype, antigen specificity, and funcation in different physiological or pathological conditions, such as infection, vaccination, allergy, autoimmune diseases, cancer, aging, and neurodegenerative diseases. TCR or BCR sequences and antigen sequences can be used as therapeutics in difference diseases or vaccine. The status of T or B cell developmental, activation status, clonal expansion status, phenotype, antigen specificity, and funcation can be used for immune profiling, disease early diagnosis, therapeutics development, prognosis, treatment progress monitoring, and treatment responder or non-responder separation.

In some embodiments, the present methods comprise the labelling of oligonucleotides barcoding antigen specificities by first covalently linking a universal DNA linker oligonucleotides or DNA handle to multimer backbone, such as dimerization antibodies or streptavidin. Then, the DNA barcode that either directly encodes the codons for amino acids in the antigen peptide or a string of random oligonucleotides that is designated to represent the identity of a particular peptide is annealed to the universal DNA linker oligonucleotides or DNA handle. This process can eliminate the need to individually covalently link DNA barcode to multimer backbone. This process can be performed in parallel for hundreds or thousands of DNA barcodes. This process can ensures that all of the DNA barcodes use the same batch of multimer backbone with the same DNA handle to multimer ratio. This process can also eliminate the DNA:multimer ratio differences if individual DNA barcodes are to be covalently linked to multimer backbone. This approach made it feasible to screen hundreds or thousands of DNA-labeled antigens at once without introducing bias to the barcode labeling ratio. This way, the true differences on antigen binding can be examined by comparing the DNA barcode aboundance without to worry about if DNA-barcode:multimer ratio introduced by individually labelling DNA barcode to multimer would causing the aboundance difference among different antigens or antigen-specific T cell number difference. This approach can also make it possible to use DNA-barcode number to separate true T cell binding antigens from background noise. This approach can also make it fast and easy to tailor a large set of different peptide antigens for different diseases or different individual patients where antigens are different. This approach can also enable the simultaneous high throughput manner, which can be easily applied in patient samples for screening thousands or tens of thousands of peptides.

In certain embodiments, the present methods allow for the quick generation of peptides using in vitro transcription and translation. This can allow one to synthesize peptide encoding oligonucleotides, which has a much faster turnaround time and a much lower cost compared to synthesizing peptides. This approach can allow make it fast and easy to tailor a large set of different peptide antigens for different diseases or different individual patients where antigens are different. This approach can also enable the simultaneous high throughput manner, which can be applied in patient samples for screening thousands or tens of thousands of peptides.

In some aspects, the methods described herein comprise the simultaneous profiling of gene expression or transcriptome, proteogenomics and TCR or BCR sequences for each single cell. This can allows for the assessment of T or B cell developmental, activation status, clonal expansion status, phenotype, antigen specificity, and funcation in different physiological or pathological conditions, such as infection, vaccination, allergy, autoimmune diseases, cancer, aging, and neurodegenerative diseases. TCR or BCR sequences and antigen sequences which can be used as therapeutics in difference diseases or vaccine. The status of T or B cell developmental, activation status, clonal expansion status, phenotype, antigen specificity, and funcation can be used for immune profiling, disease early diagnosis, therapeutics development, prognosis, treatment progress monitoring, and treatment responder or non-responder separation.

In certain aspects, the methods described herein can be used for scalable analysis for different amounts of cells as well as cells with different frequency in existence, such as antigen-specific CD8+ T cells existed at a frequency of 1 in a million CD8+ T cells or 1 in 100 CD8+ T cells. For rare antigen specific T or B cells or primary antigen specific T or B cells, plate-based single cell sequencing methods can be used while high throughput single cell gene expression analysis platforms can be used for thousands or tens of thousands of antigen specific T or B cells.

In some embodiments, the present disclosure provides methods for generating peptide MHC (pMHC) multimers for T cell isolation. First, an antigen is prepared by performing in vitro transcription/translation on a barcoded peptide-encoding oligonucleotide. The nascent peptide is then loaded into a MHC monomers, generating a pMHC. Loading may be performed by peptide exchange, such as UV-mediated peptide exchange, temperature-based peptide exchange or other methods. Several pMHC monomers with identical known peptides are then linked to a polymer conjugate which is also linked to an oligonucleotide encoding the peptide now associated with the MHC monomer, as well as a barcode. The polymer conjugate may be a dextran or a polypeptide. The pMHC multimers may further comprise a fluorophore or other detectable moiety which may aid in detection and sorting. The fluorophore may be phycoerythrin (PE), allophycocyani (APE), PE-Cy5, PE-Cy7, APC, APC-Cy7, QDOT® 565, QDOT® 605, QDOT® 655, QDOT® 705, BRILLIANT® VIOLET (BV) 421, BV 605, BV 510, BV 711, BV786, PERCP, PERCP/CY5.5, ALEXAFLUOR® 488, ALEXAFLUOR® 647, FITC, BV570, BV650, DYLIGNT® 488, DYLIGHT® 649, OR PE/DAZZLE® 594. The pMHC multimers generated as above may then be used to interrogate any antigen binding cells, such as T cells. T cells can bind the peptides of the pMHC multimers and thus these pMHC multimers can be used to isolate or stain T cells, such as by FACS. By maintaining the association of the pMHC multimers with the T cells, they may be sequenced together, thereby linking the TCR sequence with its antigen. The library preparation and sequencing can be done in a highly multiplexed fashion by preparing sequencing libraries from pMHC bound T cells which have been FACS sorted into individual wells simultaneously, and subsequently pooled for sequencing. The barcodes included in the pMHC multimers cam increase sequencing accuracy and allow for background reduction. This method accurately pairs T cell receptors with their antigens in a highly multiplexed and cost effective manner. The sequencing of the TCRs is referred to herein as Tetramer associated TCR Sequencing (TetTCR-Seq). Binding may be determined using a library of DNA-barcoded antigen-tetramers that are rapidly and inexpensively generated using an in vitro transcription/translation platform. TetTCR-Seq is effective for rapidly isolating TCR sequences that are only neoantigen-specific with no cross-reactivity to corresponding wildtype-antigens. Thus, in another method, there is provided a method for identifying neoantigen-specific T cell receptors. pMHC multimers comprising neoantigen or wild type peptides are generated using the methods presented herein, and used to stain a plurality of T cells. These pMHC multimers may be labelled so as to distinguish neoantigen presenting pMHC multimers from wild type during sorting. For example, these multimers may be labelled using different fluorophores. These pMHC bound T cells are then sorted and sequenced. T cells which only bind the neoantigen peptides can then be sequenced to identify neoantigen-specific TCRs. This method may be used over the course of immune therapy, so as to monitor the response to therapy. The neoantigen specific T cells may then be used to prepare populations of the specific neoantigen specific T cells. These populations of T cells may then be used to treat a subject, for example, a subject having cancer.

In another method, there is provided a method for identifying antigen cross-reactivity in naïve T cells. Antigen cross-reactivity can have severe consequences, so it is important for therapeutic purposes that the antigen binding repertoire of T cells is known. To begin, a plurality of pMHC multimers which present either neoantigens or wild type antigens may be used to stain naïve T cells, and sorted. The TCR sequences, and associated neoantigen sequences may then determined by sequencing. This data can then be used to help determine the course of treatment for an individual, whether by T cell therapy, or neoantigen based therapy.

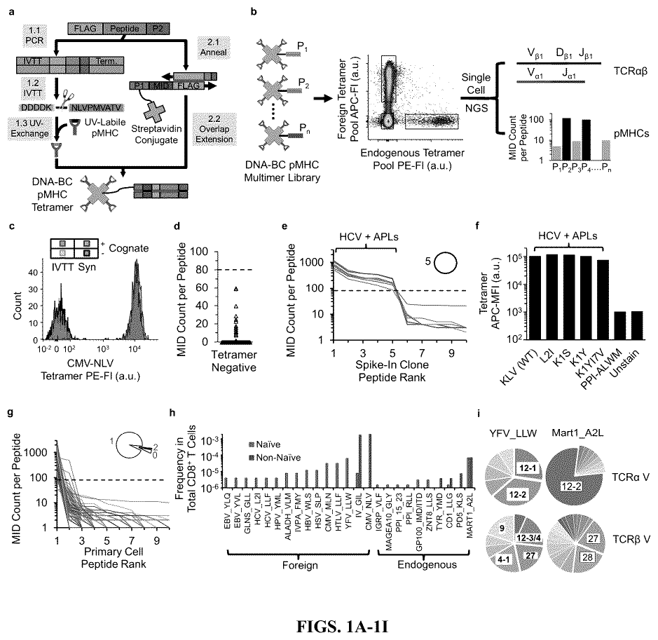

In some embodiments, there are provided methods for examining antigen-specific T cell frequency using TetTCR-seq to detect a disease or disorder. The TetTCR-seq may be applied to a sample, such as blood or other biological sample, obtained from a subject, particularly a human. The TetTCR-seq may be used to detect infection (e.g., CMV, EBV, HBV, HCV, HPV, and influenza), vaccination, and/or disease history of a subject. For example, the T cell frequency of a viral antigen or cancer antigen may be determined as shown in FIG. 1.

In another method, there is provided a method for 3′ end sequencing of RNA from a plurality of single cells. 3′ end sequencing is a method for gene expression profiling, but present methods have limited accuracy and biased sequencing depth among all cells analyzed. The method provided herein is based on the Smart-seq2 method (Picelli et al., 2013), though incorporates cellular barcodes in the reverse transcription primer to increase throughput and accuracy, and a restriction site in the template switch oligonucleotide. The reverse transcription primers comprising cellular barcodes are added to individual wells prior to cells, thereby discriminating individual cells at the library preparation stage. Cleavage of the restriction site prior to library preparation, followed by custom library preparation using the cleaved site, greatly increases 3′ end enrichment. These libraries can then be pooled and sequenced, and the gene expression can be profiled from a multitude of cells with high accuracy. Single cell 3′ end RNA-seq library can be re-pooled to adjust sequencing depth for each individual cell, thus achieving even read depth distribution among all cells analyzed. This method may be further used to analyze any cell type. Of particular interest is the gene expression of T cells, such as those isolated by the methods described herein.

In further embodiments, there are provided methods for combining the TetTCR-seq to obtain antigen specificity and TCR sequences with the T cell activation and developmental status by 3′ end single cell RNA-sequencing. The combination may be used to obtain an integrated T cell profile. The integrated T cell profile may be used to determine the presence of a disease or disorder, such as an infection, vaccination response, or cancer immunotherapy response.

Thus, the current method of TetTCR-seq may be used to obtain the T Cell Receptor (TCR) sequence and the peptide sequence of the peptide Major Histocompatibility Complex (pMHC) that the TCR binds. In addition, TetTCR-seq may be used to identify TCR cross-reactivity in a high-throughput manner. The method may be used for identifying non-crossreactive TCR sequences that react with cancer neoantigen epitopes, but not with the wildtype endogeneous epitope. Using a TCR transgenic cell lines or T cell clones generated from primary T cells, this method can also be used to identify a large peptide library to find out all possible cross-reactive peptide that a T cell may have. The read out may be sorting single T cells in either 96 well plates or 384 well plate and using multiplex PCR. A variation of this method can also be used to screen of MHC binding from pool of in vitro transcription/translation generated peptides. In addition, TetTCR-seq can be made high throughput by single cell droplet sequencing to interrogate even large number of T cells.

Further, the TetTCR-seq may be used to select the best peptide or peptide combinations and/or TCR and TCR combinations, immune monitoring on infection, vaccination, auto-immune diseases, and/or cancer. These methods may further comprise patient evaluation on which therapy to use for infection, to identify the vaccination, for tracking therapy efficacy, infection, or vaccination efficacy, and/or for post-trial analysis of patient stratification, such as responder and non-responders T cell signatures. These may be performed based on TCR clonality and antigen specificity. The 3′end scRNA-seq may be further used to reveal T cell activation and developmental status. Thus, the TetTCR-seq may be combined with in tube 3′end scRNA-seq, BD Rhapsody or 10× genomic's CHROMIUM systems, which may be high throughput.

The methods provided herein may be used to detect self-antigen specific T cells, wherein the self-antigen specific T cells cause severe adverse effect after immune checkpoint blockade therapy and other cancer immunotherapy, before a subject is administered a therapy. Also provided herein is a method of detecting T cell binding epitopes and further developing the T cell binding epitopes into vaccines or TCR redirected adoptive T cell therapy for any pathogens. Further, some embodiments provide a method of using common pathogen and auto-immune disease associated epitopes identified according to the present methods to test and monitor the immune health of individuals and predict individual's protective capacity to infection or likelihood of developing auto-immune diseases and monitoring the early on-set of auto-immune diseases. In addition, there is provided a method of detecting regulatory T cell binding epitopes according to the present methods and developing vaccines to eliminate or enhance regulator T cell function or number for immunological diseases.

### I. Definitions

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a T cell therapy comprising T cells bearing high affinity TCR(s) or a mixture of neo-antigen peptides as a vaccine or immune checkpoint blockade.

“Subject” and “patient” refer to either a human or non-human, such as primates, mammals, and vertebrates. In particular embodiments, the subject is a human.

The term “antibody” herein is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc. and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

“T cell” as used herein denotes a lymphocyte that is maintained in the thymus and has either α:β or γ:δ heterodimeric receptor. There are Va, νβ, Vy and V8, Ja, Iβ, Jy and J5, and \ddot{υ}β and 'Oδ loci. Naive T cells have not encountered specific antigens and T cells are naive when leaving the thymus. Naive T cells are identified as CD45RO″, CD45RA⁺, and CD62L⁺. Memory T cells mediate immunological memory to respond rapidly on re-exposure to the antigen that originally induced their expansion and can be “CD8⁺” (T cytotoxic cells) or “CD4⁺” (T helper cells). Memory CD4 T cells are identified as CD4⁺, CD45RO⁺ cells and memory CD8 cells are identified as CD8⁺ CD45RO⁺. In some aspects, “precursor T cells” refers to cells found in individuals without an immune response to antigen targets. The antigen targets may be HIV-specific T cells in healthy HIV negative blood donors or pre-proinsulin-specific T cells in healthy blood donors who are not diabetic.

“T cell receptor” (TCR) refers to a molecule found on the surface of T cells (or T lymphocytes) that, in association with CD3, is generally responsible for recognizing antigens bound to major histocompatibility complex (MHC) molecules. The TCR has a disulfide-linked heterodimer of the highly variable α and β chains (also known as TCRα and TCRβ, respectively) in most T cells. In a small subset of T cells, the TCR is made up of a heterodimer of variable γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al., 1997). TCR as used in the present disclosure may be from various animal species, including human, mouse, rat, or other mammals. A TCR may be cell-bound or in soluble form.

TCRs of this disclosure can be “immunospecific” or capable of binding to a desired degree, including “specifically or selectively binding” a target while not significantly binding other components present in a test sample.

“Major histocompatibility complex molecules” (MHC molecules) refer to glycoproteins that deliver peptide antigens to a cell surface. MHC class I molecules are heterodimers consisting of a membrane spanning a chain and a non-covalently associated β2 microglobulin. MHC class II molecules are composed of two transmembrane glycoproteins, a and J, both of which span the membrane. Each chain has two domains. MHC class I molecules deliver peptides originating in the cytosol to the cell surface, where the peptide:MHC complex is recognized by CD8+ T cells. MHC class II molecules deliver peptides originating in the vesicular system to the cell surface, where they are recognized by CD4+ T cells. An MHC molecule may be from various animal species, including human, mouse, rat, or other mammals.

“Peptide antigen” refers to an amino acid sequence, ranging from about 7 amino acids to about 25 amino acids in length that is specifically recognized by a TCR, or binding domains thereof, as an antigen, and which may be derived from or based on a fragment of a longer target biological molecule (e.g., polypeptide, protein) or derivative thereof. An antigen may be expressed on a cell surface, within a cell, or as an integral membrane protein. An antigen may be a host-derived (e.g., tumor antigen, autoimmune antigen) or have an exogenous origin (e.g., bacterial, viral).

“MHC-peptide tetramer staining” refers to an assay used to detect antigen-specific T cells, which features a tetramer of MHC molecules, each comprising an identical peptide having an amino acid sequence that is cognate (e.g., identical or related to) at least one antigen, wherein the complex is capable of binding T cells specific for the cognate antigen. Each of the MHC molecules may be tagged with a biotin molecule. Biotinylated MHC/peptides are tetramerized by the addition of streptavidin, which is typically fluorescently labeled. The tetramer may be detected by flow cytometry via the fluorescent label. The fluorescent label, or fluorophore, may be phycoerythrin (PE), allophycocyani (APE), PE-Cy5, PE-Cy7, APC, APC-Cy7, Qdot® 565, Qdot® 605, Qdot® 655, Qdot® 705, Brilliant® Violet (BV) 421, BV 605, BV 510, BV 711, BV786, PerCP, PerCP/Cy5.5, AlexaFluor® 488, AlexaFluor® 647, FITC, BV570, BV650, DyLignt® 488, Dylight® 649, PE/Dazzle® 594.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g. adenine “A,” guanine “G,” thymine “T” and cytosine “C”) or RNA (e.g. A, G, uracil “U” and C). The term “nucleic acid” encompasses the terms “oligonucleotide” and “polynucleotide.” The term “oligonucleotide” refers to at least one molecule of between about 3 and about 100 nucleobases in length. The term “polynucleotide” refers to at least one molecule of greater than about 100 nucleobases in length. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss”, a double-stranded nucleic acid by the prefix “ds”, and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example, and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, cells in leukapheresis chamber, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

“Analogous” forms of purines and pyrimidines are well known in the art, and include, but are not limited to aziridinylcytosine, 4-acetylcytosine, 5-fluorouracil, 5-bromouracil, 5-carboxymethylaminomethyl-2-thiouracil, 5-carboxymethylaminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N.sup.6-methyladenine, 7-methylguanine, 5-methylaminomethyluracil, 5-methoxyaminomethyl-2-thiouracil, beta-D-mannosylqueosine, 5-methoxyuracil, 2-methylthio-N6-isopentenyladenine, uracil-5-oxyacetic acid methylester, pseudouracil, queosine, 2-thiocytosine, 5-methyl-2-thiouracil, 2-thiouracil, 4-thiouracil, 5-methyluracil, uracil-5-oxyacetic acid, and 2,6-diaminopurine. The nucleic acid molecule can also contain one or more hypermodified bases, for example and without limitation, 5-hydroxymethyluracil, 5-hydroxyuracil, a-putrescinylthymine, 5-hydroxymethylcytosine, 5-hydroxycytosine, 5-methylcytosine, ˜-methyl cytosine, 2-aminoadenine, acarbamoylmethyladenine, N′-methyladenine, inosine, xanthine, hypoxanthine, 2,6-diaminpurine, and N₇-methylguanine. The nucleic acid molecule can also contain one or more non-natural bases, for example and without limitation, 7-deaza-7-hydroxymethyladenine, 7-deaza-7-hydroxymethylguanine, isocytosine (isoC), 5-methylisocytosine, and isoguanine (isoG). The nucleic acid molecule containing only canonical, hypermodified, non-natural bases, or any combinations the bases thereof, can also contain, for example and without limitation where each linkage between nucleotide residues can consist of a standard phosphodiester linkage, and in addition, may contain one or more modified linkages, for example and without limitation, substitution of the non-bridging oxygen atom with a nitrogen atom (i.e., a phosphoramidate linkage, a sulfur atom (i.e., a phosphorothioate linkage), or an alkyl or aryl group (i.e., alkyl or aryl phosphonates), substitution of the bridging oxygen atom with a sulfur atom (i.e., phosphorothiolate), substitution of the phosphodiester bond with a peptide bond (i.e., peptide nucleic acid or PNA), or formation of one or more additional covalent bonds (i.e., locked nucleic acid or LNA), which has an additional bond between the 2′-oxygen and the 4′-carbon of the ribose sugar.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

“Incorporating,” as used herein, means becoming part of a nucleic acid polymer.

“Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.”

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is generally single-stranded for maximum efficiency in amplification, but may alternatively be double-stranded. If double-stranded, the primer can first be treated to separate its strands before being used to prepare extension products. This denaturation step is typically affected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al, editors, PCR: A Practical Approach and PCR2: A Practical Approach (IRL Press, Oxford, 1991 and 1995, respectively).

“Nested PCR” refers to a two-stage PCR wherein the amplicon of a first PCR becomes the sample for a second PCR using a new set of primers, at least one of which binds to an interior location of the first amplicon. As used herein, “initial primers” or “first set of primers” in reference to a nested amplification reaction mean the primers used to generate a first amplicon, and “secondary primers” or “second set of primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999) (two-color real-time PCR). Usually, distinct sets of primers are employed for each sequence being amplified.

The term “barcode” refers to a nucleic acid sequence that is used to identify a single cell or a subpopulation of cells. Barcode sequences can be linked to a target nucleic acid of interest during amplification and used to trace back the amplicon to the cell from which the target nucleic acid originated. A barcode sequence can be added to a target nucleic acid of interest during amplification by carrying out PCR with a primer that contains a region comprising the barcode sequence and a region that is complementary to the target nucleic acid such that the barcode sequence is incorporated into the final amplified target nucleic acid product (i.e., amplicon). Barcodes can be included in either the forward primer or the reverse primer or both primers used in PCR to amplify a target nucleic acid.

The term “molecular identifier” (or “MID”) as used herein refers to a unique nucleotide sequence that is used to distinguish between a single cell or genome or a subpopulation of cells or genomes, and to distinguish duplicate sequences arising from amplification from those which are biological duplicates. MIDs may also be used to count the occurrences of specific, tagged sequences for absolute molecular counting. A MID can be linked to a target nucleic acid of interest by ligation prior to amplification, or during amplification (e.g., reverse transcription or PCR), and used to trace back the amplicon to the genome or cell from which the target nucleic acid originated. A MID can be added to a target nucleic acid by including the sequence in the adaptor to be ligated to the target. A MID can also be added to a target nucleic acid of interest during amplification by carrying out reverse transcription with a primer that contains a region comprising the barcode sequence and a region that is complementary to the target nucleic acid such that the barcode sequence is incorporated into the final amplified target nucleic acid product (i.e., amplicon). The MID may be any number of nucleotides of sufficient length to distinguish the MID from other MID. For example, a MID may be anywhere from 4 to 20 nucleotides long, such as 5 to 11, or 12 to 20. In particular aspects, the MID has a length of 6 random nucleotides. The term “molecular identifier,” “MID,” “molecular identification sequence,” “MIS,” “unique molecular identifier,” “UMI,” “molecular barcode,” “molecular identifier sequence”, “molecular tag sequence” and “barcode” are used interchangeably herein.

“Sample” means a material obtained or isolated from a fresh or preserved biological sample or synthetically-created source that contains nucleic acids of interest. In certain embodiments, a sample is the biological material that contains the variable immune region(s) for which data or information are sought. Samples can include at least one cell, fetal cell, cell culture, tissue specimen, blood, cells in leukapheresis chamber, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals, other animals, plants, fungi, bacteria, and viruses.

### II. Antigen-Specific T Cell Isolation

Certain embodiments of the present disclosure concern obtaining a population of antigen-specific T cells which are used to determine the TCR sequence. Particularly, the present disclosure relates to a substantially pure antigen-specific T cell population having a functional status which is substantially unaltered by a purification procedure comprising staining the desired T cell population, isolating the stained T cell population from a sample comprising non-stained T cell population and removing said stain, i.e. the functional status of the T cell population before purification is substantially the same as after the purification. In particular aspects, a T cell population is provided which is substantially free from any binding reagents used for the isolation of the population, e.g. antibodies or TCR binding ligands such as multimeric TCR binding ligands. The T cells may be from an in vitro culture, or a physiologic sample. For the most part, the physiologic samples employed will be blood or lymph, but samples may also involve other sources of T cells, particularly where T cells may be invasive. Thus, other sites of interest are tissues, or associated fluids, as in the brain, lymph node, neoplasms, spleen, liver, kidney, pancreas, tonsil, thymus, joints, and synovia. Prior treatments may involve removal of cells by various techniques, including centrifugation, using Ficoll-Hypaque, panning, affinity separation, using antibodies specific for one or more markers present as surface membrane proteins on the surface of cells, or any other technique that provides enrichment of the set or subset of cells of interest.

A. Starting Population of T Cells

A starting population of T cells can be obtained from a patient sample or from a healthy blood donor. In some aspects, the sample is a blood sample such as peripheral blood sample or cells in leukapheresis chamber. The blood sample can be about 1 mL to about 500 mL, such as about 2 mL to 80 mL, such as about 50 mL. The sample can include at least 500 antigen-specific T cells, at least 250 antigen-specific T cells, at least 100 antigen-specific T cells or at least 10 antigen-specific T cells.

In some embodiments, the T cells are derived from the blood, bone marrow, lymph, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells). In one embodiment, the cells (e.g., CD8⁺ cells or CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L.

In some embodiments, T cells are separated from a PBMC sample or cells in leukapheresis chamber by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 10 to about 30 days, such as about 15 to about 28 days.

The cultured T cells can be pooled and rapidly expanded. Rapid expansion provides an increase in the number of antigen-specific T-cells of at least about 50-fold (e.g., 50-, 60-, 70-, 80-, 90-, 100-, 150-fold or greater) over a period of about 10 to about 28 days. In particular, rapid expansion provides an increase of at least about 200-fold (e.g., 200-, 300-, 400-, 500-, 600-, 700-, 800-, 900-, 1000-fold or greater) over a period of about 10 to about 28 days. In some aspects, the TCR affinity is measured and/or sequence is obtained from T cells, such as tumor infiltrating lymphocytes with or without in vitro expansion.

B. Antigens

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP). The tumor-associated antigen may be a testis antigen or germline cancer antigen, such as MAGE-A1, MAGE-A3, MAGE-A4, NY-ESO-1, PRAME, CT83 and SSX2.

Other tumor associated antigens include Plu-1, HASH-1, HasH-2, Cripto and Criptin. Additionally, a tumor antigen may be a self peptide hormone, such as whole length gonadotrophin hormone releasing hormone (GnRH, International Patent Publication No. WO 95/20600), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp1OO, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A 10, MAGE-A12, MART-1, BAGE, DAM-6, -10, GAGE-1, -2, -8, GAGE-3, -4, -5, -6, -7B, NA88-A, MART-1, MC1R, Gp1OO, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (POKs), TRK receptors, PRAME, P15, RU1, RU2, SART-1, SART-3, Wilms' tumor antigen (WTi), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDK 2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (HI, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al, 2009).

Antigens derived from Epstein-Ban virus (EBV) that are contemplated for use in certain embodiments include EBV lytic proteins gp350 and gp1 1O, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or nonstructural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, pi coma virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GENBANK®, SWISS-PROT®, and TREMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (Zysk et al, 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacterium polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides), Proteus polypeptides, Pseudomonas polypeptides, Rickettsia polypeptides, Rochalimaea polypeptides, Salmonella polypeptides, Shigella polypeptides, Staphylococcus polypeptides, group Astreptococcus polypeptides (e.g., S. pyogenes M proteins), group B streptococcus (S. agalactiae) polypeptides, Treponema polypeptides, and Yersinia polypeptides (e.g., Y. pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofllaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides, (e.g., P. falciparum circumsporozoite (PfCSP)), sporozoite surface protein 2 (PfSSP2), carboxyl terminus of liver state antigen 1 (PfLSAl c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

In some embodiments, the antigen is an autoantigen. In one embodiment, the autoantigen is a type 1 diabetes autoantigen, including, but not limited to, insulin, pre-insulin, PTPRN, PDX1, ZnT8, CHGA IAAP, GAD(65) and/or DiaPep277. In one embodiment, the autoantigen is an alopecia areata autoantigen, including, but not limited to, keratin 16, K18585, M1 0510, J01523, 022528, D04547, 005529, B20572 and/or F11552. In one embodiment, the autoantigen is a systemic lupus erythematosus autoantigen, including, but not limited to, TRIM21/Ro52/SS-A 1 and/or histone H2B. In one embodiment, the autoantigen is a Behcet's disease autoantigen, including, but not limited to, S-antigen, alpha-enolase, selenium binding partner and/or Sipl C-ter. In one embodiment, the autoantigen is a Sjogren's syndrome autoantigen, including, but not limited to, La/SSB, KLK11 and/or a 45-kd nucleus protein. In one embodiment, the autoantigen is a rheumatoid arthritis autoantigen, including, but not limited to, vimentin, gelsolin, alpha 2 HS glycoprotein (AHSG), glial fibrillary acidic protein (GFAP), alB-glycoprotein (A1BG), RA33 and/or citrullinated 31F4G1. In one embodiment, the autoantigen is a Grave's disease autoantigen. In one embodiment, the autoantigen is an antiphospholipid antibody syndrome autoantigen, including, but not limited to, zwitterionic phospholipids, phosphatidyl-ethanolamine, phospholipid-binding plasma protein, phospholipid-protein complexes, anionic phospholipids, cardiolipin, β2-glycoprotein I (β2GPI), phosphatidylserine, lyso(bis)phosphatidic acid, phosphatidylethanolamine, vimentin and/or annexin A5. In one embodiment, the autoantigen is a multiple sclerosis autoantigen, including, but not limited to, myelin-associated oligodendrocytic basic protein (MOBP), myelin basic protein (MBP), myelin proteolipid protein (PLP), myelin oligodendrocyte glycoprotein (MOG) and/or alpha-B-crytallin. In one embodiment, the autoantigen is an irritable bowel disease autoantigen, including, but not limited to, a ribonucleoprotein complex, a small nuclear ribonuclear polypeptide A and/or Ro-5,200 kDa. In one embodiment, the autoantigen is a Crohn's disease autoantigen, including, but not limited to, zymogen granule membrane glycoprotein 2 (GP2), an 84 by allele of CTLA-4 AT repeat polymorphism, MRP 8, MRP 14 and/or complex MRP8/14. In one embodiment, the autoantigen is a dermatomyositis autoantigen, including, but not limited to, aminoacyl-tRNA synthetases, Mi-2 helicase/deacetylase protein complex, signal recognition particle (SRP), T2F1-Y, MDAS, NXP2, SAE and/or HMGCR. In one embodiment, the autoantigen is an ulcerative colitis autoantigen, including, but not limited to, 7E12H12 and/or M(r) 40 kD autoantigen.

In some embodiments, the autoantigen is a collagen, e.g., collagen type II; other collagens such as collagen type IX, collagen type V, collagen type XXVII, collagen type XVIII, collagen type IV, collagen type IX; aggrecan I; pancreas-specific protein disulphide isomerise A2; interphotoreceptor retinoid binding protein (IRBP); a human IRBP peptide 1-20; protein lipoprotein; insulin 2; glutamic acid decarboxylase (GAD) 1 (GAD67 protein), BAFF, IGF2. Further examples of autoantigens include ICA69 and CYP1A2, Tph and Fabp2, Tgn, Spt1 & 2 and Mater, and the CB11 peptide from collagen.

In some aspects, the peptide antigens are continuous segments of a protein. In other aspects, the peptide antigen comprises multiple segments from the same or different proteins. The multiple segments can bind to MHC and form a linear peptide sequence. The peptide sequence may be informatically predicted to bind to a certain MHC allele. The peptide sequence may be experimentally validated.

C. Isolation by DNA-pMHC Multimers

In some embodiments, the present disclosure provides a DNA-pMHC multimer for isolation of antigen-specific T cells. The DNA-pMHC multimer may comprise a multimer backbone, multiple pMHCs, and a peptide-encoding oligonucleotide, optionally comprising a DNA handle comprise a DNA barcode.

The multimer backbone may comprise multiple protein subunits to which MHC, a peptide-encoding oligonucleotide, and/or a DNA barcode are attached. The multimer backbone may comprise 2-20 subunits, such as 2, 3, 4, 5, 6, 7, 8, 9, 10, 11, 12, 13, 14, 15, 16, 17, 18, 19, or 20 subunits. The protein subunits may be comprised of streptavidin or a glucan, such as dextran.

The multimer backbone may be attached to 2 or more MHCs, such as 2-20, such as 2, 3, 4, 5, 6, 7, 8, 9, 10, 11, 12, 13, 14, 15, 16, 17, 18, 19, or 20 MHCs. In particular aspects, the multimer backbone is a tetramer, pentamer, octamer, or dodecamer. The MHC may be a class I MHC, a class II MHC, a CD1, or a MHC-like molecule. For MHC class I the presenting peptide is a 9-1 1 mer peptide; for MHC class II, the presenting peptide is 12-18mer peptides. For alternative MHC-molecules it may be fragments from lipids or gluco-molecules which are presented. In some aspects, the multimer backbone is a PROS@ MHC Class I Pentamer (ProImmune), a dodecamer comprising a biotinylated scaffold protein linked to four streptavidin tetramers, each capable of binding three biotinylated pMHC monomers (Huang et al., PNAS, 113(13); E1890-E1897, 2016), a MHC I streptamer (Iba), or a MHC-dextramer (Immudex).

In some aspects, the multimer backbone is a tetravalent conjugates (e.g., MHC I STREPTAMERS®) which comprise four identical subunits of a single ligand (e.g., peptide-major histocompatibility complexes (pMHC)) which specifically binds to the TCR and has a detectable label.

The multimer backbone may be attached to one or more peptide-encoding oligonucleotides. The peptide encoded by the oligonucleotide preferably has the same sequence as the peptide for the peptide of the pMHC complex. The peptide-encoding oligonucleotide may be linked to the multimer backbone through a DNA handle, referred to herein as a DNA oligonucleotide segment comprising at least one primer set for amplifying the oligonucleotide. The DNA handle may further encode a partial FLAG peptide. In particular aspects, the DNA handle further comprises a 10-14, such as 12, base pair degenerate region that serves as a unique molecular identifier or barcode. In some embodiments, there is provided a multimer backbone linked to a DNA handle. Thus, the peptide maybe be identified by sequencing rather than flow cytometry.

Further provided herein are methods for producing a DNA-pMHC multimer comprising the multimer backbone attached to multiple MHCs and the peptide-encoding oligonucleotide which can comprise the DNA handle. The peptide of the pMHC may have a length of about 8 to about 25 amino acids and may comprise anchor amino acid residues capable of allele-specific binding to a predetermined MHC molecule class, e.g. an MHC class I, an MHC class II or a non-classical MHC class. In particular aspects, the MHC molecule is an MHC class I molecule. Included in the HLA proteins are the class II subunits HLA-DPa, HLA-\ddot{υ}Pβ, HLA-DQa, HLA-DQ, HLA-DRa and HLA-DR, and the class I proteins HLA-A, HLA-B, HLA-C, and β2-microglobulin. The peptides of the pMHC complex may have a sequence derived from a wide variety of proteins. The T cell epitopic sequences from a number of antigens are known in the art. Alternatively, the epitopic sequence may be empirically determined, by isolating and sequencing peptides bound to native MHC proteins, by synthesis of a series of peptides from the target sequence, then assaying for T cell reactivity to the different peptides, or by producing a series of binding complexes with different peptides and quantitating the T cell binding. Alternatively, the epitopic sequence may be informatically predicted to bind to certain MHC alleles. Preparation of fragments, identifying sequences, and identifying the minimal sequence is described in U.S. Pat. No. 5,019,384; incorporated herein by reference. The peptides may be prepared in a variety of ways. Conveniently, they can be synthesized by conventional techniques employing automatic synthesizers, or may be synthesized manually. Alternatively, DNA sequences can be prepared which encode the particular peptide. The peptides may be generated by in vitro transcription/translation from the known DNA sequence. Alternatively, the DNA sequence may be cloned and expressed to provide the desired peptide. In this instance a methionine may be the first amino acid. In addition, peptides may be produced by recombinant methods as a fusion to proteins that are one of a specific binding pair, allowing purification of the fusion protein by means of affinity reagents, followed by proteolytic cleavage, usually at an engineered site to yield the desired peptide (see, e.g., Driscoll et al., 1993). The peptides may also be isolated from natural sources and purified by known techniques, including, for example, chromatography on ion exchange materials, separation by size, immunoaffinity chromatography and electrophoresis.

In one embodiment, a synthetic single-stranded DNA oligonucleotide that encodes the peptide is obtained and is utilized as a DNA template to produce the peptide using in vitro transcription/translation (IVTT) (Shimzu et al., Nat Biotechnol, 19(8): 751-5, 2001) and as the peptide-encoding oligonucleotide attached to the DNA-pMHC multimer.

For the IVTT, the peptide-encoding oligonucleotide may be amplified by polymerase chain reaction (PCR) to include adapters that allows for IVTT. The peptide-encoding sequence may comprise a partial FLAG peptide at the N-terminus, followed by the peptide of interest. During IVTT, enterokinase may be added to the solution to cleave off the FLAG peptide so that peptides without a methionine at the P1 position of the N-terminus can be produced. After IVTT, a biotinylated pMHC monomer containing a temporary peptide, such as a UV-cleavable peptide, may be added to the solution. The temporary peptide can then be switched with the target peptide.

In some aspects, MHC monomers can be generated which allow for conditional release of the MHC ligand, such as by UV irradiation (Rodenko et al., 2006) for switching the temporary and target peptides. This UV switching method comprises exposing the solution to UV light, allowing for dissociation of the temporary UV-cleavable peptide and association of the MHC with the target peptide produced by IVTT.

In other aspects, the exchange of the temporary peptide may be by chemical methods, such as biorthogonal cleavage and exchange by employing azobenzene-containing peptides (Choo et al., Angewandte Chemie International Edition, 53(49), 2014). In another method, the peptide of the pMHC may be exchanged with the target peptide by re-folding of the MHC protein in the presence of the target peptide to produce the desired pMHC (Leisner et al., PLOS One, 2008). Alternatively, the pMHC may be generated by using CLIP peptide exchange for MHC Class II (Day et al., J Clin Invest, 112)6) 831-42, 2003). In some aspects, the pMHCs may be generated by using the QUICKSWITCH™ Custom Tetramer Kit or the FLET-T™ Kit. In other aspects, the peptide of the pMHC may be exchanged with the target peptide by temperature change of the MHC protein in the presence of the target peptide to produce the desired pMHC (Luimstra et al., 2018).

In the second part of the method for producing the DNA-pMHC multimer, the peptide-encoding oligonucleotide may be annealed to a linker oligonucleotide (or DNA handle) and gap-filled using a polymerase to create a double-stranded fragment. The peptide-encoding oligonucleotide or DNA handle may be attached to the multimer backbone by methods known in the art, such as through covalent interactions, such as by a HyNic-4FB crosslink or Tetrazine-TCO crosslink, or by streptavidin-biotin interactions. In one method, the DNA handle is attached to the multimer backbone using SOLULINK®. The multimer backbone, such as streptavidin tetramer, and the oligonucleotide may be added at a molar ratio of 0.1-20, such as 3-7, such as 0.1, 3, 4, 5, 5.8, 6, or, 7, or more or fewer multimers to each oligonucleotide. The excess oligonucleotide may be removed by wash steps, such as 1, 2, 3, 4, 5, 6, 7, 8, 9, or 10, particularly 6, wash steps in a protein concentrator.

In one specific method, the linker oligonucleotide or DNA handle itself is already covalently linked to a R-phycoerythrin-streptavidin or Allophycocyanin-streptavidin conjugate. The linker sequence or DNA handle may comprise of (1) a region that's complementary to the peptide-encoding oligonucleotide, (2) a 12 base pair degenerate region that serves as a unique molecular identifier, and (3) a primer region. The resulting product is a MHC multimer, such as a fluorescent streptavidin conjugate, that is covalently linked to a double stranded DNA fragment containing the peptide-encoding sequence.

To create the final DNA-pMHC tetramer, the pMHC multimer, such as a fluorescent streptavidin conjugate, from the second part of the method is added to the IVTT solution in the first part of the method that contains the biotinylated pMHC to produce the final DNA-pMHC tetramer.

The multimer backbone may be labeled by one or more detectable labels, such as one or more fluorophores. Exemplary fluorophores include PE, PE-Cy5, PE-Cy7, APC, APC-Cy7, Qdot 565, qdot 605, Qdot 655, Qdot 705, Brilliant Violet (BV) 421, BV 605, BV 510, BV 711, BV786, PerCP, PerCP/Cy5.5, Alexa Fluor 488, Alexa Fluor 647, FITC, BV570, BV650, DyLignt 488, Dylight 649, and PE/Dazzle 594.

The labeled pMHC multimer may be free in solution, or may be attached to an insoluble support. Examples of suitable insoluble supports include beads, e.g. magnetic beads, membranes and microliter plates. These are typically made of glass, plastic (e.g. polystyrene), polysaccharides, nylon or nitrocellulose. In general, the label will have a light detectable characteristic. Preferred labels are fluorophores, such as fluorescein isothiocyanate (FITC), rhodamine, Texas Red, phycoerythrin and allophycocyanin. Other labels of interest may include dyes, enzymes, chemiluminescers, particles, radioisotopes, nucleic acids or other directly or indirectly detectable agent.

A number of methods for detection and quantitation of labeled cells are known in the art. Flow cytometry is a convenient means of enumerating cells that are a small percent of the total population. Fluorescent microscopy may also be used. Various immunoassays, e.g. ELISA, RIA, etc. may be used to quantitate the number of cells present after binding to an insoluble support. In particular aspects, flow cyometry is used for the separation of a labeled subset of T cells from a complex mixture of cells.

Alternative means of separation utilize the binding complex bound directly or indirectly to an insoluble support, e.g. column, microtiter plate, magnetic beads, etc. The cell sample is added to the binding complex. The complex may be bound to the support by any convenient means. After incubation, the insoluble support is washed to remove non-bound components. From one to six washes may be employed, with sufficient volume to thoroughly wash non-specifically bound cells present in the sample. The desired cells are then eluted from the binding complex. In particular the use of magnetic particles to separate cell subsets from complex mixtures is described in Miltenyi et al, 1990.

In some embodiments, the T cells which bind the specific pMHC can then be isolated by sorting for the detectable label. The separation of T cell, from other sample components, e.g. unstained T cells may be effected by conventional methods, e.g. cell sorting, preferably by FACS methods using commercially available systems (e.g. FACSVantage by Becton Dickinson or Moflo by Cytomation), or by magnetic cell separation (e.g. MACS by Miltenyi). The staining may be removed from the T cell by disruption of the reversible bond which results in a complete removal of any reagent bound to the target cell, because the bond between the receptor-binding component and the receptor on the target cell is a low-affinity interaction.

Further provided herein are methods of using the DNA-pMHC multimer by contacting it to T cells. T cells bearing a TCR that binds to the particular target pMHC will bind to the DNA-pMHC multimer. The T cell bound-DNA-pMHC multimer is then sorted into lysis buffer based on the detectable label, such as fluorescence. An amplification scheme may then be used to prepare a DNA library, consisting of both the TCR sequence and the DNA barcode, which can be sequenced using next generation sequencing platforms (TetTCR-seq).

The TetTCR-seq may be used to identify non-cross reactive, neoantigen-specific TCR sequences. DNA-pMHC multimers containing the neoantigen peptide are produced in one fluorescent channel (e.g., Allophycocyanin/R-Phycoerythrin), and the corresponding DNA-pMHC multimer containing the wildtype peptide are produced in another fluorescent channel. Multiple neoantigen/wildtype DNA-pMHC multimer pairs can be included in the same two fluorescent channels and in the same staining solution, since the peptide can be deconvoluted at the sequence level.

### III. TCR Sequencing

Methods are also provided herein for the sequencing of the TCR. In some embodiments, methods are provided for the simultaneous sequencing of TCRα and TCRβ genes, DNA-barcode encoding for antigenic peptide sequences, and amplification of transcripts of functional interest in the single T cells which enable linkage of TCR specificity with information about T cell function. The methods generally involve sorting of single T cells into separate locations (e.g., separate wells of a multi-well titer plate) followed by nested polymerase chain reaction (PCR) amplification of nucleic acids encoding TCRs, DNA-barcode encoding for antigenic peptide sequences and T cell phenotypic markers. The amplicons are barcoded to identify their cell of origin, combined, and analyzed by deep sequencing.

In one method, a nested PCR approach is used in combination with deep sequencing such as described in Han et al., incorporated herein by reference, with modifications. Briefly, single T cells are sorted into separate wells (e.g., 96- or 384-well PCR plate) and reverse transcription is performed using TCR primers and phenotyping primers. In order to amplify unknown TCR sequences, ligation anchor PCR may be used. One amplification primer is specific for a TCR constant region. The other primer is ligated to the terminus of cDNA synthesized from TCR encoding mRNA. The variable region is amplified by PCR between the constant region sequence and the ligated primer. Included in this first reaction are also primers to serve as hybridization locations for barcoding primers in subsequent amplification reactions. Next, nested PCR is performed with TCRα/TCR primers (e.g., sequences in Table 1) and a third reaction is performed to incorporate individual barcodes. The products are combined, purified and sequenced using a next generation sequencing platform, such as but not limited to the Illumina® HiSEQ™ system (e.g., HiSEQ2000™ and HiSEQIOOO™), the MiSEQ™ system and SOLEXA sequencing, Helicos True Single Molecule Sequencing (tSMS), the Roche 454 sequencing platform and Genome Sequencer FLX systems, the Life Technology SOLiD sequencing platform and IonTorrent system, the single molecule, real-time (SMRT™) technology of Pacific Bioscience, and nanopore sequencing. The resulting paired-end sequencing reads are assembled and deconvoluted using barcode identifiers at both ends of each sequence by a custom software pipeline to separate reads from every well in every plate. For TCR sequences, the CDR3 nucleotide sequences are then extracted and translated.

### IV. Production of T Cell Lines

Methods are also provided herein for the generation of T cell lines. In some embodiments, methods are provided for the generation of T cell lines using a DNA-BC pMHC multimer pool. The methods will generally involve separation of T cells from PBMCs, concentration, stimulation of T cells with DNA-BC pMHC multimers comprising antigens of interest, and sorting them by flow cytometry. Stimulated T cells may then be cultured for use in subsequent experiments.

In one method, T cell lines are generated according to previously published protocol (Yu et al., 2015; Zhang et al., 2016), but using the DNA-BC pMHC multimer pool to stimulate and provide a functional fluorophore for subsequent separation. Cells may then be gated by flow cytometry. Single or 5 or more cells from the same population (Neo⁺WT⁻, Neo⁻WT⁺, Neo⁺WT⁺) may be sorted into each well for subsequent culture.

### V. RNA Sequencing

RNA sequencing (RNA-seq) is a well-established method for analyzing gene expression. A variety of methodologies for RNA-seq exist. See, for example, U.S. patent application Ser. No. 14/912,556, U.S. Pat. No. 5,962,272, both of which are incorporated herein by reference. Generally, methods for RNA-seq begin by generating a cDNA from the RNA by reverse transcription. In this process, a primer is hybridized to the 3′ end of the RNA, and a reverse transcriptase extends from the primer, synthesizing complementary DNA. A second primer then hybridizes to the 3′ end of the nascent cDNA, and either a DNA polymerase, or the same reverse transcriptase extends from the primer, and synthesizes a complementary strand, thereby generating double stranded DNA, after which logarithmic amplification can begin (i.e. PCR). Many methods of cDNA synthesis utilize the poly(A) tail of the mRNA as the starting point for cDNA synthesis and utilize a first primer which has a stretch of T nucleotides, complementary to the poly(A) tail. Some methods then use random primers as the other primers, though this has proved to cause consistent bias. As practiced in U.S. patent application Ser. No. 14/912,556 and U.S. Pat. No. 5,962,272, certain reverse transcriptases can add extra non-templated nucleotides to the end of a sequence, and then switch templates to a primer which binds there. This allows for the addition of the second primer, with very low bias.

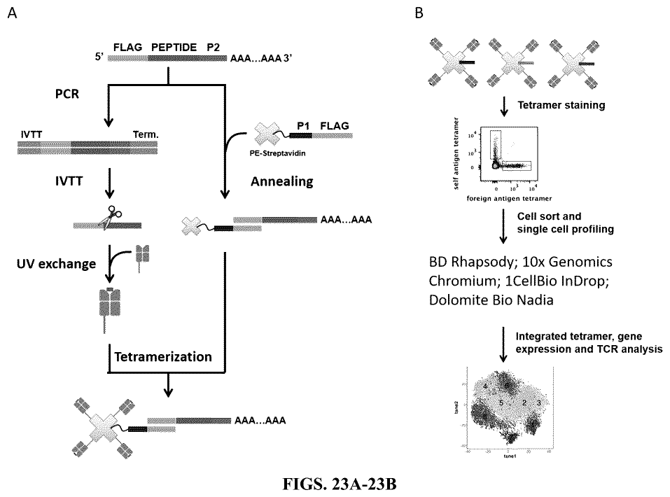

Further embodiments of the present disclosure concern highly multiplexed 3′ end RNA sequencing to analyze the gene expression of a plurality of single cells (FIG. 23). These methods use the template switch activity of particular reverse transcriptases, as described above, to add a template switch primer comprising a restriction endonuclease site. The reverse transcription (RT) primer includes a cellular barcode and a restriction enzyme (e.g., SalI or Spel) site is incorporated on the template switching oligo (TSO). In one method, the RT primer and the template switch primer comprise the sequences in Table 1. RT primers with unique cell barcodes may then be individually dispensed into wells. These wells may be in a 96-, 384, or nanowell plate. Target cells are then sorted by FACS, adding single cells to each well or by dispersing. These cells are then lysed. cDNA amplification is performed similarly to the Smart-Seq2 protocol, but with the primers provided in Table 1 (Picelli et al., 2013). After cDNA amplification, multiple single cell PCR products are pooled, each of which has the unique cell barcode at the 3′ end to differentiate the individual cells during analysis. After purification, PCR products are digested by restriction enzyme incubation. Digested products may be used for preparing a DNA library, such as by using a modified Nextera XT DNA library prep kit, where custom primers designed to enrich 3′ end are used to prepare sequencing libraries.

TABLE 1
Oligo Sequences.

| Oligo # | Oligo sequences 5′ to 3′ |
|---|---|
| SEQ ID | /5AmMC12//iSp18/ TAG TAC TCA GAG GTT GAT CTA CAT TG (N:25252525)(N)(N) (N)(N)(N) |
| NO. 1 | (N)(N)(N)(N)(N)(N) GAC GAT GAC GAC AAG |
| SEQ ID | GCG AAT TAA TAC GAC TCA CTA TAG GGC TTA AGT ATA AGG AGG AAA ACA T ATG GAC GAT |
| NO. 2 | GAC GAC AAG |
| SEQ ID | AAA CCC CTC CGT HA GAG AGG GGT TA TGC TAG CGA GGT GCT TCG TTA |
| NO. 3 | |
| SEQ ID | TCA GAG GH GAT CTA CAT TG |
| NO. 4 | |
| SEQ ID | AG CGA GGT GCT TCG TTA |
| NO. 5 | |
| SEQ ID | GACGTGTGCTCTTCCGATCT NHNHN ATCACG TAC TCA GAG GTT GAT CTA CAT TG |
| NO. 6 | |
| SEQ ID | GACGTGTGCTCTTCCGATCT NHNHN CGATGT TAC TCA GAG GTT GAT CTA CAT TG |
| NO. 7 | |
| SEQ ID | GACGTGTGCTCTTCCGATCT NHNHN TTAGGC TAC TCA GAG GTT GAT CTA CAT TG |
| NO. 8 | |
| SEQ ID | GACGTGTGCTCTTCCGATCT NHNHN TGACCA TAC TCA GAG GTT GAT CTA CAT TG |
| NO. 9 | |
| SEQ ID | GACGTGTGCTCTTCCGATCT NHNHN ACAGTG TAC TCA GAG GTT GAT CTA CAT TG |
| NO. 10 | |
| SEQ ID | GACGTGTGCTCTTCCGATCT NHNHN GCCAAT TAC TCA GAG GTT GAT CTA CAT TG |
| NO. 11 | |
| SEQ ID | GACGTGTGCTCTTCCGATCT NHNHN CAGATC TAC TCA GAG GTT GAT CTA CAT TG |
| NO. 12 | |
| SEQ ID | GACGTGTGCTCTTCCGATCT NHNHN ACTTGA TAC TCA GAG GTT GAT CTA CAT TG |
| NO. 13 | |
| SEQ ID | GACGTGTGCTCTTCCGATCT NHNHN GATCAG TAC TCA GAG GTT GAT CTA CAT TG |
| NO. 14 | |
| SEQ ID | GACGTGTGCTCTTCCGATCT NHNHN TAGCTT TAC TCA GAG GTT GAT CTA CAT TG |
| NO. 15 | |
| SEQ ID | GACGTGTGCTCTTCCGATCT NHNHN GGCTAC TAC TCA GAG GTT GAT CTA CAT TG |
| NO. 16 | |
| SEQ ID | GACGTGTGCTCTTCCGATCT NHNHN CTTGTA TAC TCA GAG GTT GAT CTA CAT TG |
| NO. 17 | |
| SEQ ID | ACACTCTTTCCCTACACGACGCTCTTCCGATCT NHNHN TCAAG AG CGA GGT GCT TCG TTA |
| NO. 18 | |
| SEQ ID | ACACTCTTTCCCTACACGACGCTCTTCCGATCT NHNHN AACAC AG CGA GGT GCT TCG TTA |
| NO. 19 | |
| SEQ ID | ACACTCTTTCCCTACACGACGCTCTTCCGATCT NHNHN ACATA AG CGA GGT GCT TCG TTA |
| NO. 20 | |
| SEQ ID | ACACTCTTTCCCTACACGACGCTCTTCCGATCT NHNHN TAAGA AG CGA GGT GCT TCG TTA |
| NO. 21 | |
| SEQ ID | ACACTCTTTCCCTACACGACGCTCTTCCGATCT NHNHN TCAAG AG CGA GGT GCT TCG TTA |
| NO. 22 | |
| SEQ ID | ACACTCTTTCCCTACACGACGCTCTTCCGATCT NHNHN AGTTT AG CGA GGT GCT TCG TTA |
| NO. 23 | |
| SEQ ID | ACACTCTTTCCCTACACGACGCTCTTCCGATCT NHNHN ATACA AG CGA GGT GCT TCG TTA |
| NO. 24 | |
| SEQ ID | ACACTCTTTCCCTACACGACGCTCTTCCGATCT NHNHN TTATG AG CGA GGT GCT TCG TTA |
| NO. 25 | |
| SEQ ID | AATGATACGGCGACCACCGAGATCTACACTCTTTCCCTACACGAC |
| NO. 26 | |
| SEQ ID | CAAGCAGAAGACGGCATACGAGATAA XXXXXX GTGACTGGAGTTCAGACGTGTGCTCTTCCGATCT (XXXXXX |
| NO. 27 | denotes cell barcodes) |
| SEQ ID | CGAGGTGCTTCGTTACAGGATGATGTTTTTGTCCATGATAGCCTTGTCGTCATCGTC |
| NO. 28 | |
| SEQ ID | CGAGGTGCTTCGTTACAGTTTAACTTTGATGTTCAGCAGAGCCTTGTCGTCATCGTC |
| NO. 29 | |
| SEQ ID | CGAGGTGCTTCGTTAAACGTGCAGAGATTTGTCCATCAGAGCCTTGTCGTCATCGTC |
| NO. 30 | |
| SEQ ID | CGAGGTGCTTCGTTACAGGTAGATGTGGTGGTCAGACAGAGCCTTGTCGTCATCGTC |
| NO. 31 | |
| SEQ ID | CGAGGTGCTTCGTTATGCTGCAGGATCAGGACCCCACAGTGCCTTGTCGTCATCGTC |
| NO. 32 | |
| SEQ ID | CGAGGTGCTTCGTTAAGCTGCTGCCGGATCAGGACCCCACAGTGCCTTGTCGTCATCGTC |
| NO. 33 | |
| SEQ ID | CGAGGTGCTTCGTTACAGCGGCAGCAGACGCATCCACAGAGCCTTGTCGTCATCGTC |
| NO. 34 | |
| SEQ ID | CGAGGTGCTTCGTTAAACTTCCATCGTGTGGGTGCCCAGCATAGCCTTGTCGTCATCGTC |
| NO. 35 | |
| SEQ ID | CGAGGTGCTTCGTTAAACGGTCCAGCAAACACCATTGATGCACTTGTCGTCATCGTC |
| NO. 36 | |
| SEQ ID | CGAGGTGCTTCGTTAAACCATCGTCAGCAGACCACCCAGGCACTTGTCGTCATCGTC |
| NO. 37 | |
| SEQ ID | CGAGGTGCTTCGTTATGCAGAGGTCTGGAAACTCCACAGCAGGCACTTGTCGTCATCGTC |
| NO. 38 | |
| SEQ ID | CGAGGTGCTTCGTTAAACAGCTTCCAGCAGCAGGTGCATACACTTGTCGTCATCGTC |
| NO. 39 | |
| SEQ ID | CGAGGTGCTTCGTTACAGGTAGAATGCGTGTTCCCACATATCCTTGTCGTCATCGTC |
| NO. 40 | |
| SEQ ID | CGAGGTGCTTCGTTAAACGGTCAGGATACCGATACCAGCCAGTTCCTTGTCGTCATCGTC |
| NO. 41 | |
| SEQ ID | CGAGGTGCTTCGTTAAACCTGGCAGATGTAAGAGTCAATGAACTTGTCGTCATCGTC |
| NO. 42 | |
| SEQ ID | CGAGGTGCTTCGTTACAGGTAGAAACCAACAGCGAACAGGAACTTGTCGTCATCGTC |
| NO. 43 | |
| SEQ ID | CGAGGTGCTTCGTTACAGAGCAACAGACAGAACGATCAGGAACTTGTCGTCATCGTC |
| NO. 44 | |
| SEQ ID | CGAGGTGCTTCGTTAAACAGACGGGAAGAAGTCAGACGGCAGGAACTTGTCGTCATCGTC |
| NO. 45 | |
| SEQ ID | CGAGGTGCTTCGTTAGATCAGCATGAAAACAGACCACAGGAACTTGTCGTCATCGTC |
| NO. 46 | |
| SEQ ID | CGAGGTGCTTCGTTACAGCAGCAGAGCCAGAGCGTACAGGAACTTGTCGTCATCGTC |
| NO. 47 | |
| SEQ ID | CGAGGTGCTTCGTTAGATTTCGTAGATGAATTTGTTCATAAACTTGTCGTCATCGTC |
| NO. 48 | |
| SEQ ID | CGAGGTGCTTCGTTAGATGAAGTGGAAGTCAGAGTACATGAACTTGTCGTCATCGTC |
| NO. 49 | |
| SEQ ID | CGAGGTGCTTCGTTAAACGTCGGTAAAGAATTCACCCACAAACTTGTCGTCATCGTC |
| NO. 50 | |
| SEQ ID | CGAGGTGCTTCGTTACAGGGTGAAAACAAAACCCAGAATGCCCTTGTCGTCATCGTC |
| NO. 51 | |
| SEQ ID | CGAGGTGCTTCGTTACAGCATAGCAACCAGCGTGCACAGGCCCTTGTCGTCATCGTC |
| NO. 52 | |
| SEQ ID | CGAGGTGCTTCGTTACAGAGACGGAGCGTGGTGCAGCAGACCCTTGTCGTCATCGTC |
| NO. 53 | |
| SEQ ID | CGAGGTGCTTCGTTACAGTTCTTCTTCCAGAGACAGCAGACCCTTGTCGTCATCGTC |
| NO. 54 | |
| SEQ ID | CGAGGTGCTTCGTTACAGGAAACGGTTCAGGTTCGGAGACAGACCCTTGTCGTCATCGTC |
| NO. 55 | |
| SEQ ID | CGAGGTGCTTCGTTACAGGTGTTCCATACCGTCGTACAGACCCTTGTCGTCATCGTC |
| NO. 56 | |
| SEQ ID | CGAGGTGCTTCGTTAAACCAGGTACAGAGCTTCAACCAGGTGCTTGTCGTCATCGTC |
| NO. 57 | |
| SEQ ID | CGAGGTGCTTCGTTAAACAGACAGAACACCGTCAACAGCCAGGATCTTGTCGTCATCGTC |
| NO. 58 | |
| SEQ ID | CGAGGTGCTTCGTTAAACACCGTGCACCGGCTCTTTCAGGATCTTGTCGTCATCGTC |
| NO. 59 | |
| SEQ ID | CGAGGTGCTTCGTTACAGTTTGTGGATGTGTTCCATCAGGATCTTGTCGTCATCGTC |
| NO. 60 | |
| SEQ ID | CGAGGTGCTTCGTTAAACGTATTGCAGATCTTGACCCGGCAGGATCTTGTCGTCATCGTC |
| NO. 61 | |
| SEQ ID | CGAGGTGCTTCGTTAAACGCCTTTGGTGATGTCGGTCAGGATCTTGTCGTCATCGTC |
| NO. 62 | |
| SEQ ID | CGAGGTGCTTCGTTAAACAGAGAACGGAACCTGGTCCATGATCTTGTCGTCATCGTC |
| NO. 63 | |
| SEQ ID | CGAGGTGCTTCGTTAAACACGTTCCAGGGCTTCCAGCATGATCTTGTCGTCATCGTC |
| NO. 64 | |
| SEQ ID | CGAGGTGCTTCGTTAAACAGAAAACGGAACTTGATCGGTGATCTTGTCGTCATCGTC |
| NO. 65 | |
| SEQ ID | CGAGGTGCTTCGTTAAACAGCGTTAATACCCAGAGCAACAATTTTCTTGTCGTCATCGTC |
| NO. 66 | |
| SEQ ID | CGAGGTGCTTCGTTACAGAACAATCAGGAACACTTGCAGTTTCTTGTCGTCATCGTC |
| NO. 67 | |
| SEQ ID | CGAGGTGCTTCGTTAAGCCAGCAGGTCACCTTCAGACAGTTTCTTGTCGTCATCGTC |
| NO. 68 | |
| SEQ ID | CGAGGTGCTTCGTTAAACAGCGTTGATACCCAGAGCAACCAGTTTCTTGTCGTCATCGTC |
| NO. 69 | |
| SEQ ID | CGAGGTGCTTCGTTACACATTGTTGATACCCAGTGCAACCAGTTTCTTGTCGTCATCGTC |
| NO. 70 | |
| SEQ ID | CGAGGTGCTTCGTTACACTTGCCAATACTGACCCCAGGTTTTCTTGTCGTCATCGTC |
| NO. 71 | |
| SEQ ID | CGAGGTGCTTCGTTACAGAGCGGTAACCTGACCAGCGCACAGCAGCTTGTCGTCATCGTC |
| NO. 72 | |
| SEQ ID | CGAGGTGCTTCGTTAAACTTCGATCAGAGCCAGACCGAACAGCAGCTTGTCGTCATCGTC |
| NO. 73 | |
| SEQ ID | CGAGGTGCTTCGTTACACATAAACCGGGTAACCAAACAGCAGCTTGTCGTCATCGTC |
| NO. 74 | |
| SEQ ID | CGAGGTGCTTCGTTAAACCCAGCCACCCAGGATGTTGAACAGCAGCTTGTCGTCATCGTC |
| NO. 75 | |
| SEQ ID | CGAGGTGCTTCGTTAAACAAACATGCAGGTTGCGCCCAGCAGCTTGTCGTCATCGTC |
| NO. 76 | |
| SEQ ID | CGAGGTGCTTCGTTACAGCAGGAAAGAGGTCAGGTCGATCAGCAGCTTGTCGTCATCGTC |
| NO. 77 | |
| SEQ ID | CGAGGTGCTTCGTTACAGCCACAGAGAGAACAGAGACAGCAGCTTGTCGTCATCGTC |
| NO. 78 | |
| SEQ ID | CGAGGTGCTTCGTTAAACAGCCATCGGACCGTTCCACAGCAGCTTGTCGTCATCGTC |
| NO. 79 | |
| SEQ ID | CGAGGTGCTTCGTTAAACATTGATAGACGGGATGTTCAGCATCTTGTCGTCATCGTC |
| NO. 80 | |
| SEQ ID | CGAGGTGCTTCGTTACAGCGGCAGCAGGTGCTGGTACAGCATCTTGTCGTCATCGTC |
| NO. 81 | |
| SEQ ID | CGAGGTGCTTCGTTAAACGGTGCAACCAGATTCCCAAACCATCTTGTCGTCATCGTC |
| NO. 82 | |
| SEQ ID | CGAGGTGCTTCGTTAAACGGTAGCCAGGTCGGTCTGAGCCAGGTTCTTGTCGTCATCGTC |
| NO. 83 | |
| SEQ ID | CGAGGTGCTTCGTTAAACGGTAGCAACCATCGGAACCAGGTTCTTGTCGTCATCGTC |
| NO. 84 | |
| SEQ ID | CGAGGTGCTTCGTTAAACAACATCCATGCACGGGATCAGCTGCTTGTCGTCATCGTC |
| NO. 85 | |
| SEQ ID | CGAGGTGCTTCGTTACAGAGAGGTCAGAGCGCACAGCAGACGCTTGTCGTCATCGTC |
| NO. 86 | |
| SEQ ID | CGAGGTGCTTCGTTACAGCAGAGCCAGCAGCGGCAGCAGACGCTTGTCGTCATCGTC |
| NO. 87 | |
| SEQ ID | CGAGGTGCTTCGTTACAGGTACGGTGCGTTCGGGAACATGCGCTTGTCGTCATCGTC |
| NO. 88 | |
| SEQ ID | CGAGGTGCTTCGTTAAACCATCGTGGTGCCATATTCCATCATACGCTTGTCGTCATCGTC |
| NO. 89 | |
| SEQ ID | CGAGGTGCTTCGTTAAACCAGGTACAGAGCTTCAACCAGATGTGACTTGTCGTCATCGTC |
| NO. 90 | |
| SEQ ID | CGAGGTGCTTCGTTAAACTTCCAGCAGACGGCCAATGATAGACTTGTCGTCATCGTC |
| NO. 91 | |
| SEQ ID | CGAGGTGCTTCGTTACAGCAGTTTAACACCAGCAGCCAGAGACTTGTCGTCATCGTC |
| NO. 92 | |
| SEQ ID | CGAGGTGCTTCGTTAAGCCTGGGTGATCCACATCAGCAGAGACTTGTCGTCATCGTC |
| NO. 93 | |
| SEQ ID | CGAGGTGCTTCGTTAAACCTGGGTGATCCACATCAGCAGAGACTTGTCGTCATCGTC |
| NO. 94 | |
| SEQ ID | CGAGGTGCTTCGTTAAGCGTAGTAAACGGTGATCGGCAGAGACTTGTCGTCATCGTC |
| NO. 95 | |
| SEQ ID | CGAGGTGCTTCGTTACAGTTCAGCCTGCAGCGGAGACAGAGACTTGTCGTCATCGTC |
| NO. 96 | |
| SEQ ID | CGAGGTGCTTCGTTAAACAGCGTTGATACCCAGAGCAACCAGAGACTTGTCGTCATCGTC |
| NO. 97 | |
| SEQ ID | CGAGGTGCTTCGTTACAGGTTAACTTCGTAAACGTGGTACAGAGACTTGTCGTCATCGTC |
| NO. 98 | |
| SEQ ID | CGAGGTGCTTCGTTACAGGGTAGCCACGGTGTTATACAGAGACTTGTCGTCATCGTC |
| NO. 99 | |
| SEQ ID | CGAGGTGCTTCGTTAAACGCCAACTTCAAAAACACGGTACATAGACTTGTCGTCATCGTC |
| NO. 100 | |
| SEQ ID | CGAGGTGCTTCGTTAAACACCCGTAATGGTACTAGCAACAGACTTGTCGTCATCGTC |
| NO. 101 | |
| SEQ ID | CGAGGTGCTTCGTTAAACAGGCATCGGAGACGGTTTTGACAGGGTCTTGTCGTCATCGTC |
| NO. 102 | |
| SEQ ID | CGAGGTGCTTCGTTAAACGGTCAGAACAATGTTAGCAGCAACCTTGTCGTCATCGTC |
| NO. 103 | |
| SEQ ID | CGAGGTGCTTCGTTAAACCAGCGGGGTCAGCATAACAATAACCTTGTCGTCATCGTC |
| NO. 104 | |
| SEQ ID | CGAGGTGCTTCGTTACAGCATAACAGAGGTTTCTTCCAGAACCTTGTCGTCATCGTC |
| NO. 105 | |
| SEQ ID | CGAGGTGCTTCGTTAGATAGCGAAACCCAGACCGAACAGAACCTTGTCGTCATCGTC |
| NO. 106 | |
| SEQ ID | CGAGGTGCTTCGTTATGCTTCCAGCAGGTCGTCGTGCAGAACCTTGTCGTCATCGTC |
| NO. 107 | |
| SEQ ID | CGAGGTGCTTCGTTATTCAACACCCGGAACACCACCCATCAGAACCTTGTCGTCATCGTC |
| NO. 108 | |
| SEQ ID | CGAGGTGCTTCGTTAAACAGCCAGAGAAGAAACAATAATCATAACCTTGTCGTCATCGTC |
| NO. 109 | |
| SEQ ID | CGAGGTGCTTCGTTAAACCACGTATTGCAGCAGGATGTTCATAACCTTGTCGTCATCGTC |
| NO. 110 | |
| SEQ ID | CGAGGTGCTTCGTTAGAGGTACACCAGAACACCAGTAACAACCTTGTCGTCATCGTC |
| NO. 111 | |
| SEQ ID | CGAGGTGCTTCGTTACAGGGTTGAAGTGCCCGGCAGTAACCACTTGTCGTCATCGTC |
| NO. 112 | |
| SEQ ID | CGAGGTGCTTCGTTAAACAGTAGAAGTACCCGGCAGTAACCACTTGTCGTCATCGTC |
| NO. 113 | |
| SEQ ID | CGAGGTGCTTCGTTAAACAAACGGAACCAGCAGAGACAGCCACTTGTCGTCATCGTC |
| NO. 114 | |
| SEQ ID | CGAGGTGCTTCGTTAAGCGGTAACCGGACCAGGTTCCAGGTACTTGTCGTCATCGTC |
| NO. 115 | |
| SEQ ID | CGAGGTGCTTCGTTAGATGTGCACGATAGCCGGTAACAGGTACTTGTCGTCATCGTC |
| NO. 116 | |
| SEQ ID | CGAGGTGCTTCGTTACAGACGCGGACCACGACGAGGCAGCAGGTACTTGTCGTCATCGTC |
| NO. 117 | |
| SEQ ID | CGAGGTGCTTCGTTACAGGGTCCACCAGTTCTGCTGCAGGTACTTGTCGTCATCGTC |
| NO. 118 | |
| SEQ ID | CGAGGTGCTTCGTTAAACAGCGTTGATACCCAGAGCAACCAGGTACTTGTCGTCATCGTC |
| NO. 119 | |
| SEQ ID | CGAGGTGCTTCGTTAAACAGCGTTAACACCCAGAGCAACCAGGTACTTGTCGTCATCGTC |
| NO. 120 | |
| SEQ ID | CGAGGTGCTTCGTTAAACCTGAGACATGGTGCCATCCATATACTTGTCGTCATCGTC |
| NO. 121 | |
| SEQ ID | CGAGGTGCTTCGTTAGGTGGTTTCCGGCTGCAGGTCCAGCATGTACTTGTCGTCATCGTC |
| NO. 122 | |
| SEQ ID | CGAGGTGCTTCGTTAAACAACAATCAGGTGGTCCAGAACATACTTGTCGTCATCGTC |
| NO. 123 | |
| SEQ ID | CGAGGTGCTTCGTTAAACAGAAACATCCAGGTAGATCAGGAACTTGTCGTCATCGTC |
| NO. 124 | |
| SEQ ID | CGAGGTGCTTCGTTACAGGTGCAGGTCAAAGTCCGGCATGAACTTGTCGTCATCGTC |
| NO. 125 | |
| SEQ ID | CGAGGTGCTTCGTTAAACACCGAACAGTTTAACACCCAGCAGAACCTTGTCGTCATCGTC |
| NO. 126 | |
| SEQ ID | CGAGGTGCTTCGTTACAGGTAGGTGTTGTGGTGGATCAGAGCCTTGTCGTCATCGTC |
| NO. 127 | |
| SEQ ID | CGAGGTGCTTCGTTAAACCAGGAAGATGGTGAAGTTTTCCAGAACCTTGTCGTCATCGTC |
| NO. 128 | |
| SEQ ID | CGAGGTGCTTCGTTACAGGAAGATGGTGAAGTTTTCCAGAACAGACTTGTCGTCATCGTC |
| NO. 129 | |
| SEQ ID | CGAGGTGCTTCGTTAAACTTCGTAGTTCAGGCCGGTCAGGATCTTGTCGTCATCGTC |
| NO. 130 | |
| SEQ ID | CGAGGTGCTTCGTTACAGAACCGGAACAAAACCGTACAGAGCCTTGTCGTCATCGTC |
| NO. 131 | |
| SEQ ID | CGAGGTGCTTCGTTAAACCGGCGGAGCCCAAGACATAACAACCTTGTCGTCATCGTC |
| NO. 132 | |
| SEQ ID | CGAGGTGCTTCGTTACAGCAGCAGAGACGGGGTTTCCAGCAGAGCCTTGTCGTCATCGTC |
| NO. 133 | |
| SEQ ID | CGAGGTGCTTCGTTAGATGTGCGGGATAACCGGAGACAGAGCCTTGTCGTCATCGTC |
| NO. 134 | |
| SEQ ID | CGAGGTGCTTCGTTAAACACCGTAAACCAGGAATTCAAACAGTTTCTTGTCGTCATCGTC |
| NO. 135 | |
| SEQ ID | CGAGGTGCTTCGTTAAACCGGAACAGAGCAGCAATTCAGGTTCTTGTCGTCATCGTC |
| NO. 136 | |
| SEQ ID | CGAGGTGCTTCGTTAGATCAGGTGGATGAACGGGATAATCAGCTTGTCGTCATCGTC |
| NO. 137 | |
| SEQ ID | CGAGGTGCTTCGTTACAGACACGGCGGCATACCAAACAGCAGCTTGTCGTCATCGTC |
| NO. 138 | |
| SEQ ID | CGAGGTGCTTCGTTACAGCAGAACCAGTTGATGAGACAGTTTCTTGTCGTCATCGTC |
| NO. 139 | |
| SEQ ID | CGAGGTGCTTCGTTAAACAGAGTAAACGTAAGAACCAACAGCCTTGTCGTCATCGTC |
| NO. 140 | |
| SEQ ID | CGAGGTGCTTCGTTAAACACGGGTCAGCAGGTTATACAGGAACTTGTCGTCATCGTC |
| NO. 141 | |
| SEQ ID | CGAGGTGCTTCGTTACAGTTTCTGCTGGATGTTCATCAGTTTCTTGTCGTCATCGTC |
| NO. 142 | |
| SEQ ID | CGAGGTGCTTCGTTACAGCGGAAACAGTTGTTCACCCAGCATCTTGTCGTCATCGTC |
| NO. 143 | |
| SEQ ID | CGAGGTGCTTCGTTAAACAGAAACGTCCAGGTAGGTCAGGAACTTGTCGTCATCGTC |
| NO. 144 | |
| SEQ ID | CGAGGTGCTTCGTTACAGGTGCAGGTCGAAGTCCGGCATAGACTTGTCGTCATCGTC |
| NO. 145 | |
| SEQ ID | CGAGGTGCTTCGTTACACACCAGACAGTTTCACACCCAGCAGAACCTTGTCGTCATCGTC |
| NO. 146 | |
| SEQ ID | CGAGGTGCTTCGTTACAGGTGGGTGTTGTGGTGGATCAGAGCCTTGTCGTCATCGTC |
| NO. 147 | |
| SEQ ID | CGAGGTGCTTCGTTAAACCAGCAGGATGGTGAAGTTTTCCAGAACCTTGTCGTCATCGTC |
| NO. 148 | |
| SEQ ID | CGAGGTGCTTCGTTACAGCAGGATGGTGAAGTTTTCCAGAACAGACTTGTCGTCATCGTC |
| NO. 149 | |
| SEQ ID | CGAGGTGCTTCGTTATGCTTCGTAGTTCAGACCAGTCAGGATCTTGTCGTCATCGTC |
| NO. 150 | |
| SEQ ID | CGAGGTGCTTCGTTACAGAACCGGAACAGAACCGTACAGAGCCTTGTCGTCATCGTC |
| NO. 151 | |
| SEQ ID | CGAGGTGCTTCGTTAAACCGGCGGAGCCCAAGACAGAACAACCTTGTCGTCATCGTC |
| NO. 152 | |
| SEQ ID | CGAGGTGCTTCGTTACAGCAGCAGAGACAGGGTTTCCAGCAGAGCCTTGTCGTCATCGTC |
| NO. 153 | |
| SEQ ID | CGAGGTGCTTCGTTAGATCAGCGGGATAACCGGAGACAGAGCCTTGTCGTCATCGTC |
| NO. 154 | |
| SEQ ID | CGAGGTGCTTCGTTACACACCGTGAACCAGGAACTCGAACAGTTTCTTGTCGTCATCGTC |
| NO. 155 | |
| SEQ ID | CGAGGTGCTTCGTTAAACCGGAACAGAGCAACGGTTCAGGTTCTTGTCGTCATCGTC |
| NO. 156 | |
| SEQ ID | CGAGGTGCTTCGTTAGATCAGGTGGATGCACGGGATAATCAGCTTGTCGTCATCGTC |
| NO. 157 | |
| SEQ ID | CGAGGTGCTTCGTTACAGGCACGGGGTCATACCGAACAGCAGCTTGTCGTCATCGTC |
| NO. 158 | |
| SEQ ID | CGAGGTGCTTCGTTACAGCAGAACCGGCTGGTGAGACAGTTTCTTGTCGTCATCGTC |
| NO. 159 | |
| SEQ ID | CGAGGTGCTTCGTTAAACAGAGTAAACGTGAGAACCAACAGCCTTGTCGTCATCGTC |
| NO. 160 | |
| SEQ ID | CGAGGTGCTTCGTTAAACACGGGTCAGCGGGTTATACAGGAACTTGTCGTCATCGTC |
| NO. 161 | |
| SEQ ID | CGAGGTGCTTCGTTACAGTTGCTGCTGGATGTTCATCAGTTTCTTGTCGTCATCGTC |
| NO. 162 | |
| SEQ ID | CGAGGTGCTTCGTTACAGCGGGAACAGACGTTCACCCAGCATCTTGTCGTCATCGTC |
| NO. 163 | |
| SEQ ID | CGAGGTGCTTCGTTACAGGAAGTGAACCAGTTCAGCAACTTTCTTGTCGTCATCGTC |
| NO. 164 | |
| SEQ ID | CGAGGTGCTTCGTTACAGGAAGTGAACCAGTTCAGCCATTTTCTTGTCGTCATCGTC |
| NO. 165 | |
| SEQ ID | CGAGGTGCTTCGTTACAGGAAGTGAACCAGTTCAACCATTTTCTTGTCGTCATCGTC |
| NO. 166 | |
| SEQ ID | CGAGGTGCTTCGTTACAGGAAGTGAACCAGTTTAGCAACTTTCTTGTCGTCATCGTC |
| NO. 167 | |
| SEQ ID | CGAGGTGCTTCGTTAGGTGAAACGCACAAATGCAAACAGGCGCTTGTCGTCATCGTC |
| NO. 168 | |
| SEQ ID | CGAGGTGCTTCGTTAGGTGTTACGGATCAGTTCATCCAGGTACTTGTCGTCATCGTC |
| NO. 169 | |
| SEQ ID | CGAGGTGCTTCGTTAAACTTCGTTACCACGGAATTGCAGGAACTTGTCGTCATCGTC |
| NO. 170 | |
| SEQ ID | CGAGGTGCTTCGTTAAACTTTTTCTTCAATATCGGTCAGGATCTTGTCGTCATCGTC |
| NO. 171 | |
| SEQ ID | CGAGGTGCTTCGTTACAGGTGCAGGTCAAAATCCGGCATGAACTTGTCGTCATCGTC |
| NO. 172 | |
| SEQ ID | CGAGGTGCTTCGTTACAGCTTCTGTTGGATGTTCATCAGTTTCTTGTCGTCATCGTC |
| NO. 173 | |
| SEQ ID | CGAGGTGCTTCGTTAAACAGGTTTGTCAACCGGAAACATACCCTTGTCGTCATCGTC |
| NO. 174 | |
| SEQ ID | CGAGGTGCTTCGTTAAACCGGAAACATACCCAGATACTGAACCTTGTCGTCATCGTC |
| NO. 175 | |
| SEQ ID | CGAGGTGCTTCGTTACAGTTCATATTCCACATGCGGTAACCACTTGTCGTCATCGTC |
| NO. 176 | |
| SEQ ID | CGAGGTGCTTCGTTAAACGTGCAACGGAGATGCCCACAGTTTCTTGTCGTCATCGTC |
| NO. 177 | |
| SEQ ID | CGAGGTGCTTCGTTACAGGGTGAAGATGTCCACATTCAGGATCTTGTCGTCATCGTC |
| NO. 178 | |
| SEQ ID | CGAGGTGCTTCGTTACAGGTGGGTAATGAAAACGTAAACAAACTTGTCGTCATCGTC |
| NO. 179 | |
| SEQ ID | CGAGGTGCTTCGTTAAATACGTGCCTGGGTCAGCAGCATGAACTTGTCGTCATCGTC |
| NO. 180 | |
| SEQ ID | CGAGGTGCTTCGTTACACACGTACTAAGGCCAGAATTGACAGCATCTTGTCGTCATCGTC |
| NO. 181 | |
| SEQ ID | CGAGGTGCTTCGTTAAACTTCTGCCGGGGTGTAAGACAGAGCCTTGTCGTCATCGTC |
| NO. 182 | |
| SEQ ID | CGAGGTGCTTCGTTAGATCAGACCCAGGTCACCGTCCATCAGATGCTTGTCGTCATCGTC |
| NO. 183 | |
| SEQ ID | CGAGGTGCTTCGTTACAGACCCAGGTCACCGTCCATCAGATGCTTGTCGTCATCGTC |
| NO. 184 | |
| SEQ ID | CGAGGTGCTTCGTTACAGAGACGGAGAATGCGGAACCATCAGCTTGTCGTCATCGTC |
| NO. 185 | |
| SEQ ID | CGAGGTGCTTCGTTATGCGTTCAGAATTTGCTCAAACAGTTTCTTGTCGTCATCGTC |
| NO. 186 | |
| SEQ ID | CGAGGTGCTTCGTTACAGTTTGGTGTGCAGGGTCAGCATGTACTTGTCGTCATCGTC |
| NO. 187 | |
| SEQ ID | CGAGGTGCTTCGTTAAATCGCAATGAAAAAAGAGGTCAGACCCTTGTCGTCATCGTC |
| NO. 188 | |
| SEQ ID | CGAGGTGCTTCGTTAAACCAGGTACAGGTGGTCAGACAGAAACTTGTCGTCATCGTC |
| NO. 189 | |
| SEQ ID | CGAGGTGCTTCGTTACAGACCAGAGAAGATAGCCAGCAGGTACTTGTCGTCATCGTC |
| NO. 190 | |
| SEQ ID | CGAGGTGCTTCGTTAAACAACTGCGGTGATGGTGTTCAGTTTCTTGTCGTCATCGTC |
| NO. 191 | |
| SEQ ID | CGAGGTGCTTCGTTACAGACCGTGAGCGTCGTCCACCAGCATCTTGTCGTCATCGTC |
| NO. 192 | |
| SEQ ID | CGAGGTGCTTCGTTATGCAACAATAACAGCCAGCATCAGCATCTTGTCGTCATCGTC |
| NO. 193 | |
| SEQ ID | CGAGGTGCTTCGTTACACAACCGCCAGCGTACCTGCTAACAGCTTGTCGTCATCGTC |
| NO. 194 | |
| SEQ ID | CGAGGTGCTTCGTTAAACACGAGGAGACAGCGGAGCCAGAGACTTGTCGTCATCGTC |
| NO. 195 | |
| SEQ ID | CGAGGTGCTTCGTTAAACGCCGAACAGTTTCACACCCAGCAGAACCTTGTCGTCATCGTC |
| NO. 196 | |
| SEQ ID | CGAGGTGCTTCGTTAAACCGTACCAACCATCGTAAACAGCGTCTTGTCGTCATCGTC |
| NO. 197 | |
| SEQ ID | CGAGGTGCTTCGTTAAACATTCGGCACGGTCATAGCCAGCAGCTTGTCGTCATCGTC |
| NO. 198 | |
| SEQ ID | CGAGGTGCTTCGTTACACATTCGGAACTTTAATTGCCAGTAACTTGTCGTCATCGTC |
| NO. 199 | |
| SEQ ID | CGAGGTGCTTCGTTAAACTTCCAGGTCGTTGATTTTGGTCATAAACTTGTCGTCATCGTC |
| NO. 200 | |
| SEQ ID | CGAGGTGCTTCGTTACAGAACAGACAGCAGATCGTTCAGGAACTTGTCGTCATCGTC |
| NO. 201 | |
| SEQ ID | CGAGGTGCTTCGTTAAATGAACCATGCAATAACCATCAGACCCTTGTCGTCATCGTC |
| NO. 202 | |
| SEQ ID | CGAGGTGCTTCGTTAAACAGCAACAACATAAGAAAAGATGAACTTGTCGTCATCGTC |
| NO. 203 | |
| SEQ ID | CGAGGTGCTTCGTTACAGATAGGTGTTGTGGTGGATCAGAGCCTTGTCGTCATCGTC |
| NO. 204 | |
| SEQ ID | CGAGGTGCTTCGTTAGATATTAGCAGCCCAGTCCAGCAGAATCTTGTCGTCATCGTC |
| NO. 205 | |
| SEQ ID | CGAGGTGCTTCGTTAAACCGGAGACAGTTCAGAGAACAGACTCTTGTCGTCATCGTC |
| NO. 206 | |
| SEQ ID | CGAGGTGCTTCGTTACAGTTCGGTGTAGTATTCCAGAACAGACTTGTCGTCATCGTC |
| NO. 207 | |
| SEQ ID | CGAGGTGCTTCGTTAAACTTCAAACAGAGATTTCGCAATATGCTTGTCGTCATCGTC |
| NO. 208 | |
| SEQ ID | CGAGGTGCTTCGTTAAACCGGCGGAGCCCAACTCATAACAACCTTGTCGTCATCGTC |
| NO. 209 | |
| SEQ ID | CGAGGTGCTTCGTTAAACGGTCACAAAAATATCCATTGCGGTCTTGTCGTCATCGTC |
| NO. 210 | |
| SEQ ID | CGAGGTGCTTCGTTAAACAAAAATGTCCATAGCGGTAACATACTTGTCGTCATCGTC |
| NO. 211 | |
| SEQ ID | CGAGGTGCTTCGTTATGCGCCAACAATCCAGGTCAGAACGTACTTGTCGTCATCGTC |
| NO. 212 | |
| SEQ ID | CGAGGTGCTTCGTTACAGAACCGGAACAAAACCATACAGTGCCTTGTCGTCATCGTC |
| NO. 213 | |
| SEQ ID | CGAGGTGCTTCGTTACAGTAACAGAGACGGGGTTTCCAGCAGTGCCTTGTCGTCATCGTC |
| NO. 214 | |
| SEQ ID | CGAGGTGCTTCGTTACAGCAGAGACGGGGTTTCCAGCAGTGCCTTGTCGTCATCGTC |
| NO. 215 | |
| SEQ ID | CGAGGTGCTTCGTTAGATCCAGTACAGCATATTGAAGATCAGCTTGTCGTCATCGTC |
| NO. 216 | |
| SEQ ID | CGAGGTGCTTCGTTAAACCGGAGAGGTGGTCAGGTCCAGAGACTTGTCGTCATCGTC |
| NO. 217 | |
| SEQ ID | CGAGGTGCTTCGTTACAGGTAGATGTTAGCCAGCGGCATTTTCTTGTCGTCATCGTC |
| NO. 218 | |
| SEQ ID | CGAGGTGCTTCGTTAAACCAGGAAGTCCAGAGAGAAAGAGAACTTGTCGTCATCGTC |
| NO. 219 | |
| SEQ ID | CGAGGTGCTTCGTTACAGCTTCACGGTGTACTTTTGCAGAAACTTGTCGTCATCGTC |
| NO. 220 | |
| SEQ ID | CGAGGTGCTTCGTTAGATTTTTGCGATCATAGCGTTCAGGATCTTGTCGTCATCGTC |
| NO. 221 | |
| SEQ ID | CGAGGTGCTTCGTTAGATGTAGGTGTGCAGTTCAGACAGTTTCTTGTCGTCATCGTC |
| NO. 222 | |
| SEQ ID | CGAGGTGCTTCGTTAAACGCTAACAGACAGTAACAGCAGAGACTTGTCGTCATCGTC |
| NO. 223 | |
| SEQ ID | CGAGGTGCTTCGTTACAGGGTCACGGTCAGTTCGGCCATATACTTGTCGTCATCGTC |
| NO. 224 | |
| SEQ ID | CGAGGTGCTTCGTTACAGTTCACCCGGAGAGTCATACATATACTTGTCGTCATCGTC |
| NO. 225 | |
| SEQ ID | CGAGGTGCTTCGTTAAACAATGTAAACAATAGAGAACGGCATCATCTTGTCGTCATCGTC |
| NO. 226 | |
| SEQ ID | CGAGGTGCTTCGTTAAATGTAAACAATAGAGAACGGCATCATCTTGTCGTCATCGTC |
| NO. 227 | |
| SEQ ID | CGAGGTGCTTCGTTAGATGTAAACAATAGAGAACGGCATCATCAGCTTGTCGTCATCGTC |
| NO. 228 | |
| SEQ ID | CGAGGTGCTTCGTTACAGGTAGAACAGGTGAGAGAAACTCATGGTCTTGTCGTCATCGTC |
| NO. 229 | |
| SEQ ID | CGAGGTGCTTCGTTACAGCAGGATAGAAATGCCCATAATGAACTTGTCGTCATCGTC |
| NO. 230 | |
| SEQ ID | CGAGGTGCTTCGTTAAACCAGGAATGCACGGTGAAACAGAACCTTGTCGTCATCGTC |
| NO. 231 | |
| SEQ ID | CGAGGTGCTTCGTTAAACCAGGTTCAGAACATCAGAAGAAAACTTGTCGTCATCGTC |
| NO. 232 | |
| SEQ ID | CGAGGTGCTTCGTTACAGAAACTCCAGATACGGAACCAGACGCTTGTCGTCATCGTC |
| NO. 233 | |
| SEQ ID | CGAGGTGCTTCGTTAAACCGGCTTGATCTCACGAGACAGTTTCTTGTCGTCATCGTC |
| NO. 234 | |
| SEQ ID | CGAGGTGCTTCGTTAAACATAGTAGGTTAAGATTGCCAGCAGCTTGTCGTCATCGTC |
| NO. 235 | |
| SEQ ID | CGAGGTGCTTCGTTAAGCGTTCACGTTCAGATCCGGCAGAAACTTGTCGTCATCGTC |
| NO. 236 | |
| SEQ ID | CGAGGTGCTTCGTTAGATCGGAGACAGGATTTCAGAGGTGTACTTGTCGTCATCGTC |
| NO. 237 | |
| SEQ ID | CGAGGTGCTTCGTTACAGAGCCAGATAGCGATTAAACAGGTTCTTGTCGTCATCGTC |
| NO. 238 | |
| SEQ ID | CGAGGTGCTTCGTTACAGCAGCCAGGTAACTGATGCGATCAGCAGCTTGTCGTCATCGTC |
| NO. 239 | |
| SEQ ID | CGAGGTGCTTCGTTACAGCCAGGTAACAGATGCGATCAGCAGCTTGTCGTCATCGTC |
| NO. 240 | |
| SEQ ID | CGAGGTGCTTCGTTAAACGCCTTCCATAAATTCGTCCAGGAACTTGTCGTCATCGTC |
| NO. 241 | |
| SEQ ID | CGAGGTGCTTCGTTAGATATGCGGGATAACCGGAGACAGAGCCTTGTCGTCATCGTC |
| NO. 242 | |
| SEQ ID | CGAGGTGCTTCGTTAAGCCAGTTGAACCGGAGGCCATAAATACTTGTCGTCATCGTC |
| NO. 243 | |
| SEQ ID | CGAGGTGCTTCGTTAAACAACACGTAACGGCTCCCATAACCACTTGTCGTCATCGTC |
| NO. 244 | |
| SEQ ID | CGAGGTGCTTCGTTACAGCAGACACGGCGGCATACCAAACAGCAGCTTGTCGTCATCGTC |
| NO. 245 | |
| SEQ ID | CGAGGTGCTTCGTTACAGACACGGCGGCATACCGAACAGCAGCTTGTCGTCATCGTC |
| NO. 246 | |
| SEQ ID | CGAGGTGCTTCGTTACAGTTTCGCAATGGTTTCATTCAGACCCTTGTCGTCATCGTC |
| NO. 247 | |
| SEQ ID | CGAGGTGCTTCGTTAAACAGGCGGCGGCATACCAATAACCAGCTTGTCGTCATCGTC |
| NO. 248 | |
| SEQ ID | CGAGGTGCTTCGTTAAACTTCCGGGCCTTTTTCGTCCAGCAGCTTGTCGTCATCGTC |
| NO. 249 | |
| SEQ ID | CGAGGTGCTTCGTTAGATAGAAGAGTAATACTGATAAATGAACTTGTCGTCATCGTC |
| NO. 250 | |
| SEQ ID | CGAGGTGCTTCGTTAAACTTCGTAGTTCAGACCCGTCAGAATCTTGTCGTCATCGTC |
| NO. 251 | |
| SEQ ID | CGAGGTGCTTCGTTACAGGGTCGGGTCAGCAGGATTCAGAATCTTGTCGTCATCGTC |
| NO. 252 | |
| SEQ ID | CGAGGTGCTTCGTTACAGGAAAGGGAACATAACAATCAGGATCTTGTCGTCATCGTC |
| NO. 253 | |
| SEQ ID | CGAGGTGCTTCGTTACATCAGGGTCAGCAGGTACAGCATGAACTTGTCGTCATCGTC |
| NO. 254 | |
| SEQ ID | CGAGGTGCTTCGTTAAACCATAACCAGGTACATGAACAGGAACTTGTCGTCATCGTC |
| NO. 255 | |
| SEQ ID | CGAGGTGCTTCGTTACAGCAGCGGGAACAGAACATTCAGGAACTTGTCGTCATCGTC |
| NO. 256 | |
| SEQ ID | CGAGGTGCTTCGTTACAGTGCCAGGTTTTCCAGAAAGATATACTTGTCGTCATCGTC |
| NO. 257 | |
| SEQ ID | CGAGGTGCTTCGTTAGGTATTATAGAACACAGCAACCATTTTCTTGTCGTCATCGTC |
| NO. 258 | |
| SEQ ID | CGAGGTGCTTCGTTACAGCATGTAGATAAACGGATTCAGAACCTTGTCGTCATCGTC |
| NO. 259 | |
| SEQ ID | CGAGGTGCTTCGTTAAACAAACACAACCAGTTCGTTCAGATACTTGTCGTCATCGTC |
| NO. 260 | |
| SEQ ID | CGAGGTGCTTCGTTAAACAACGGTCACGGTGTAGATTTCCAGGAACTTGTCGTCATCGTC |
| NO. 261 | |
| SEQ ID | CGAGGTGCTTCGTTAAACGGTCACGGTATAGATTTCCAGGAACTTGTCGTCATCGTC |
| NO. 262 | |
| SEQ ID | CGAGGTGCTTCGTTAGATGAATGCGAAAAAGGTGAACAGGAACTTGTCGTCATCGTC |
| NO. 263 | |
| SEQ ID | CGAGGTGCTTCGTTAGATAGCCAGCAGATAGCAGTCAATGAACTTGTCGTCATCGTC |
| NO. 264 | |
| SEQ ID | CGAGGTGCTTCGTTAAACGTGCGGAGAACCTTGCAGCAGAGACTTGTCGTCATCGTC |
| NO. 265 | |
| SEQ ID | CGAGGTGCTTCGTTACAGCGGGAACAGTTGTTCACCCAGCATCTTGTCGTCATCGTC |
| NO. 266 | |
| SEQ ID | CGAGGTGCTTCGTTAAACAAACAGCAGAACCAGGAACAGGAACTTGTCGTCATCGTC |
| NO. 267 | |
| SEQ ID | CGAGGTGCTTCGTTAAACGCCCATAACCAGCGGAAAAACCAGCTTGTCGTCATCGTC |
| NO. 268 | |
| SEQ ID | CGAGGTGCTTCGTTACAGCGGAAAAACCAGATCATGCAGACGCTTGTCGTCATCGTC |
| NO. 269 | |
| SEQ ID | CGAGGTGCTTCGTTAAACAGAGTAAACATAAGAACCAACAGCCTTGTCGTCATCGTC |
| NO. 270 | |
| SEQ ID | CGAGGTGCTTCGTTATGCCGGAAAGAAGATAATGCTCAGCAGCTTGTCGTCATCGTC |
| NO. 271 | |
| SEQ ID | CGAGGTGCTTCGTTACATGAAATGAGAGAAAACGGTCAGGAACTTGTCGTCATCGTC |
| NO. 272 | |
| SEQ ID | CGAGGTGCTTCGTTATGCAGATGAGAATGCAGCGAACAGTAACTTGTCGTCATCGTC |
| NO. 273 | |
| SEQ ID | CGAGGTGCTTCGTTACAGACCCCACAGAGAAACCAGTAATTGCTTGTCGTCATCGTC |
| NO. 274 | |
| SEQ ID | CGAGGTGCTTCGTTAAACTTCCACAACCACACCCAGTTGATGCTTGTCGTCATCGTC |
| NO. 275 | |
| SEQ ID | CGAGGTGCTTCGTTAAACACGTTGAACGGCATCCAGAATAAACTTGTCGTCATCGTC |
| NO. 276 | |
| SEQ ID | CGAGGTGCTTCGTTACAGAGAGTTATGATATTCAGACAGTTTCTTGTCGTCATCGTC |
| NO. 277 | |
| SEQ ID | CGAGGTGCTTCGTTAAATGAATTTGAAGTTCTGGTCTGCTAACAGCTTGTCGTCATCGTC |
| NO. 278 | |
| SEQ ID | CGAGGTGCTTCGTTACAGGTACGGTTTGAAATAATTCAGAACCTTGTCGTCATCGTC |
| NO. 279 | |
| SEQ ID | CGAGGTGCTTCGTTAAATAGAAGAAATTGCGCCAACCAGAGCCTTGTCGTCATCGTC |
| NO. 280 | |
| SEQ ID | CGAGGTGCTTCGTTAAACACGGGTCAGCAGGTTATACAGAAACTTGTCGTCATCGTC |
| NO. 281 | |
| SEQ ID | CGAGGTGCTTCGTTAAACCGGGGTACTGATTTCAACAATGTGCTTGTCGTCATCGTC |
| NO. 282 | |
| SEQ ID | CGAGGTGCTTCGTTAAACAATTTCAACACCAGCCAGCAGTTTCTTGTCGTCATCGTC |
| NO. 283 | |
| SEQ ID | CGAGGTGCTTCGTTAAACGGTGTGGACAACCTGTTCGCCCAGAATCTTGTCGTCATCGTC |
| NO. 284 | |
| SEQ ID | CGAGGTGCTTCGTTACAGAAAAACCAGTGAACCCGCCATTGCCTTGTCGTCATCGTC |
| NO. 285 | |
| SEQ ID | CGAGGTGCTTCGTTAAGCTGCAATGATGGTGGTCGGCATGTACTTGTCGTCATCGTC |
| NO. 286 | |
| SEQ ID | CGAGGTGCTTCGTTACATACCAAAAATCTGTGCAACCAGGATCTTGTCGTCATCGTC |
| NO. 287 | |
| SEQ ID | CGAGGTGCTTCGTTACAGACCCAGAACTTGCGTAATCAGAATCTTGTCGTCATCGTC |
| NO. 288 | |
| SEQ ID | CGAGGTGCTTCGTTACAGACCCAGGAACAGAGCAGCCAGGATACGCTTGTCGTCATCGTC |
| NO. 289 | |
| SEQ ID | CGAGGTGCTTCGTTACAGCAGAACCGTCCAAGAACCCAGCAGCTTGTCGTCATCGTC |
| NO. 290 | |
| SEQ ID | CGAGGTGCTTCGTTAAACGCCGTAAACCAGGAACTCAAACAGTTTCTTGTCGTCATCGTC |
| NO. 291 | |
| SEQ ID | CGAGGTGCTTCGTTAGGTGTACGGCAGCGGGTTAGCCAGTTTCTTGTCGTCATCGTC |
| NO. 292 | |
| SEQ ID | CGAGGTGCTTCGTTACAGCAGAACCAGTTGGTGAGACAGTTTCTTGTCGTCATCGTC |
| NO. 293 | |
| SEQ ID | CGAGGTGCTTCGTTACAGACCGATTGCTTCGTCCAGCAGGAACTTGTCGTCATCGTC |
| NO. 294 | |
| SEQ ID | CGAGGTGCTTCGTTAGGTGGTAGCCATAGAATCTTGCAGATACTTGTCGTCATCGTC |
| NO. 295 | |
| SEQ ID | CGAGGTGCTTCGTTACAGGAAAGAAGAGATAGATGCCATCAGGAACTTGTCGTCATCGTC |
| NO. 296 | |
| SEQ ID | CGAGGTGCTTCGTTAGAAAGAAGAGATAGATGCCATCAGGAACTTGTCGTCATCGTC |
| NO. 297 | |
| SEQ ID | CGAGGTGCTTCGTTACAGAAAACTTGAAATAGATGCCATCAGCTTGTCGTCATCGTC |
| NO. 298 | |
| SEQ ID | CGAGGTGCTTCGTTATGCCGAGAAATGCAGAGCGAACAGCAGCTTGTCGTCATCGTC |
| NO. 299 | |
| SEQ ID | CGAGGTGCTTCGTTAAATACCCGGATAATGCTTAATCAGACGCTTGTCGTCATCGTC |
| NO. 300 | |
| SEQ ID | CGAGGTGCTTCGTTACAGAACACCAGAATAGCTGCTCATAAACTTGTCGTCATCGTC |
| NO. 301 | |
| SEQ ID | CGAGGTGCTTCGTTAAACCATTGCCAGCAGCGGACCCATACCCTTGTCGTCATCGTC |
| NO. 302 | |
| SEQ ID | CGAGGTGCTTCGTTACAGAAAGATGGTGAAGTTTTCCAGAACAGACTTGTCGTCATCGTC |
| NO. 303 | |
| SEQ ID | CGAGGTGCTTCGTTAAACCAGAAAGATGGTGAAGTTTTCCAGAACCTTGTCGTCATCGTC |
| NO. 304 | |
| SEQ ID | CGAGGTGCTTCGTTACATATTGGTTTCCAGGGTCATCAGAAACTTGTCGTCATCGTC |
| NO. 305 | |
| SEQ ID | CGAGGTGCTTCGTTAAACAACATAGAAAGAAACTGCAAACGTAACCTTGTCGTCATCGTC |
| NO. 306 | |
| SEQ ID | CGAGGTGCTTCGTTACAGCAGTAAGGTAACTTGCAGTAATGCCTTGTCGTCATCGTC |
| NO. 307 | |
| SEQ ID | CGAGGTGCTTCGTTAAACAGATGCAGCGTGTTCAGAGGTGTACTTGTCGTCATCGTC |
| NO. 308 | |
| SEQ ID | CGAGGTGCTTCGTTAGGTTTCCAGGAAGGTTTCAGCCAGAGACTTGTCGTCATCGTC |
| NO. 309 | |
| SEQ ID | CGAGGTGCTTCGTTAAACGGTGTTAGAGATAGCTGCCATCGTCTTGTCGTCATCGTC |
| NO. 310 | |
| SEQ ID | CGAGGTGCTTCGTTACAGCGGAACAGACGGAGATGCCAGAAACTTGTCGTCATCGTC |
| NO. 311 | |
| SEQ ID | CGAGGTGCTTCGTTAAACAGACGGAGATGCCAGGAACATATACTTGTCGTCATCGTC |
| NO. 312 | |
| SEQ ID | CGAGGTGCTTCGTTACAGAGACACATCATGTTTCAGCAGCATCTTGTCGTCATCGTC |
| NO. 313 | |
| SEQ ID | CGAGGTGCTTCGTTACAGAACAATTAACATATTCAGCAGTAACTTGTCGTCATCGTC |
| NO. 314 | |
| SEQ ID | CGAGGTGCTTCGTTACAGAGCAGAGGTATAACCGATCATAAACTTGTCGTCATCGTC |
| NO. 315 | |
| SEQ ID | CGAGGTGCTTCGTTAGGTGTAACCAATCATAAACAGCAGGTACTTGTCGTCATCGTC |
| NO. 316 | |
| SEQ ID | CGAGGTGCTTCGTTAAACCGGATCAATGTCCAGCGGCAGTTTCTTGTCGTCATCGTC |
| NO. 317 | |
| SEQ ID | CGAGGTGCTTCGTTAGATTTCAAAACTCTGGTTCAGTTGGAACTTGTCGTCATCGTC |
| NO. 318 | |
| SEQ ID | CGAGGTGCTTCGTTAGATCAGGTGAATAAACGGGATAATCAGCTTGTCGTCATCGTC |
| NO. 319 | |
| SEQ ID | CGAGGTGCTTCGTTAAATTGAGCTACTTGCCCAGAACATTAACTTGTCGTCATCGTC |
| NO. 320 | |
| SEQ ID | CGAGGTGCTTCGTTAGATCAGGTACAGGTGTGAGATAATCATCTTGTCGTCATCGTC |
| NO. 321 | |
| SEQ ID | CGAGGTGCTTCGTTAAACAGAAACATCCAGGTAAATCAGGAACTTGTCGTCATCGTC |
| NO. 322 | |
| SEQ ID | CGAGGTGCTTCGTTAAACAGAAACATTGAAAATCAGCAGTAACTTGTCGTCATCGTC |
| NO. 323 | |
| SEQ ID | CGAGGTGCTTCGTTAAACAAACAGATTCATCCACAGCAGGCTCTTGTCGTCATCGTC |
| NO. 324 | |
| SEQ ID | CGAGGTGCTTCGTTACACATGATACCATTTTTCCTGGGTGAACTTGTCGTCATCGTC |
| NO. 325 | |
| SEQ ID | CGAGGTGCTTCGTTAAACAGAAATGTCCTGAATAAACAGATTCTTGTCGTCATCGTC |
| NO. 326 | |
| SEQ ID | CGAGGTGCTTCGTTAGGTGTTTTTAATCAGTTCGTCCAGGTACTTGTCGTCATCGTC |
| NO. 327 | |
| SEQ ID | CGAGGTGCTTCGTTAAACTTCGTTACCACGAGATTGCAGGAACTTGTCGTCATCGTC |
| NO. 328 | |
| SEQ ID | CGAGGTGCTTCGTTAAACTTTTTCTTCCATGTCGGTCAGGATCTTGTCGTCATCGTC |
| NO. 329 | |
| SEQ ID | CGAGGTGCTTCGTTACAGGTGCAGGTCGAAGTCCGGCATAGACTTGTCGTCATCGTC |
| NO. 330 | |
| SEQ ID | CGAGGTGCTTCGTTACAGTTGCTGCTGGATGTTCATCAGTTTCTTGTCGTCATCGTC |
| NO. 331 | |
| SEQ ID | CGAGGTGCTTCGTTAAACAGGTTTGTCAACCGGCAGCATACCCTTGTCGTCATCGTC |
| NO. 332 | |
| SEQ ID | CGAGGTGCTTCGTTAAACCGGCAGCATACCCAGATACTGAACCTTGTCGTCATCGTC |
| NO. 333 | |
| SEQ ID | CGAGGTGCTTCGTTACAGTTCATATTCCACGTGCGGTAAACGCTTGTCGTCATCGTC |
| NO. 334 | |
| SEQ ID | CGAGGTGCTTCGTTAAACGTGTAACGGAGATGCGCCCAGTTTCTTGTCGTCATCGTC |
| NO. 335 | |
| SEQ ID | CGAGGTGCTTCGTTACAGGGTGAAAACGTCCACATTCAGGATCTTGTCGTCATCGTC |
| NO. 336 | |
| SEQ ID | CGAGGTGCTTCGTTACAGGTGGGTGGTGAAAACGTAAACAAACTTGTCGTCATCGTC |
| NO. 337 | |
| SEQ ID | CGAGGTGCTTCGTTACAGACGTGCCTGGGTCAGCAGCATGAACTTGTCGTCATCGTC |
| NO. 338 | |
| SEQ ID | CGAGGTGCTTCGTTACACACCAACCAGAGCCAGGATAGACAGCATCTTGTCGTCATCGTC |
| NO. 339 | |
| SEQ ID | CGAGGTGCTTCGTTAAACTTCAACCGGGGTGTAAGACAGAGCCTTGTCGTCATCGTC |
| NO. 340 | |
| SEQ ID | CGAGGTGCTTCGTTAGATCAGACCCAGGTCACCGTCCATCAGGTTCTTGTCGTCATCGTC |
| NO. 341 | |
| SEQ ID | CGAGGTGCTTCGTTACAGACCCAGGTCACCGTCCATCAGGTTCTTGTCGTCATCGTC |
| NO. 342 | |
| SEQ ID | CGAGGTGCTTCGTTACAGAGACGGAGAGTGCAGAACCATCAGCTTGTCGTCATCGTC |
| NO. 343 | |
| SEQ ID | CGAGGTGCTTCGTTATGCTTTCAGGATCTGCTCAAACAGTTTCTTGTCGTCATCGTC |
| NO. 344 | |
| SEQ ID | CGAGGTGCTTCGTTACAGTTTGGTACGCAGGGTCAGCATGTACTTGTCGTCATCGTC |
| NO. 345 | |
| SEQ ID | CGAGGTGCTTCGTTAGATAGCGATAACAAAAGAGGTCAGACCCTTGTCGTCATCGTC |
| NO. 346 | |
| SEQ ID | CGAGGTGCTTCGTTAAACCAGGTACGGGTGGTCAGACAGGAACTTGTCGTCATCGTC |
| NO. 347 | |
| SEQ ID | CGAGGTGCTTCGTTACAGACCAGAGAAGATAGCGAACAGGTACTTGTCGTCATCGTC |
| NO. 348 | |
| SEQ ID | CGAGGTGCTTCGTTAAACAACCGGGGTGATGGTGTTCAGTTTCTTGTCGTCATCGTC |
| NO. 349 | |
| SEQ ID | CGAGGTGCTTCGTTACAGACCGTGAGCGTCGTCCACCAGAACCTTGTCGTCATCGTC |
| NO. 350 | |
| SEQ ID | CGAGGTGCTTCGTTATGCAACAATAACAGCGAACATCAGCATCTTGTCGTCATCGTC |
| NO. 351 | |
| SEQ ID | CGAGGTGCTTCGTTACACTCCCGCCAGCGTACCTGCTAACAGCTTGTCGTCATCGTC |
| NO. 352 | |
| SEQ ID | CGAGGTGCTTCGTTATGCACGAGGAGACAGCGGAGCCAGAGACTTGTCGTCATCGTC |
| NO. 353 | |
| SEQ ID | CGAGGTGCTTCGTTAAACGCCAGACAGTTTCACACCCAGCAGAACCTTGTCGTCATCGTC |
| NO. 354 | |
| SEQ ID | CGAGGTGCTTCGTTAAACGGTGCCCACAATGGTAAACAGGGTCTTGTCGTCATCGTC |
| NO. 355 | |
| SEQ ID | CGAGGTGCTTCGTTAAACATTCGGAACTTTCATAGCCAGCAGCTTGTCGTCATCGTC |
| NO. 356 | |
| SEQ ID | CGAGGTGCTTCGTTAAACTTCCAGACCGTTGATTTTGGTCATAAACTTGTCGTCATCGTC |
| NO. 357 | |
| SEQ ID | CGAGGTGCTTCGTTACATAACAGACAGCAGGTCGTTCAGGAACTTGTCGTCATCGTC |
| NO. 358 | |
| SEQ ID | CGAGGTGCTTCGTTAAATGAACCATGCGATAGCCATCAGACCCTTGTCGTCATCGTC |
| NO. 359 | |
| SEQ ID | CGAGGTGCTTCGTTAAACAGCAACAACATAAGAGATGATGAACTTGTCGTCATCGTC |
| NO. 360 | |
| SEQ ID | CGAGGTGCTTCGTTACAGGTGGGTGTTGTGGTGGATCAGAGCCTTGTCGTCATCGTC |
| NO. 361 | |
| SEQ ID | CGAGGTGCTTCGTTAAACATTAGCAGCCCAGTCCAGCAGAATCTTGTCGTCATCGTC |
| NO. 362 | |
| SEQ ID | CGAGGTGCTTCGTTAAACCGGAGACAGTTCAGAGAACAGAGCCTTGTCGTCATCGTC |
| NO. 363 | |
| SEQ ID | CGAGGTGCTTCGTTACAGTTCGGTGTAGTATTCCAGCAGAGACTTGTCGTCATCGTC |
| NO. 364 | |
| SEQ ID | CGAGGTGCTTCGTTAAACTTCAAACGGAGATTTCGCAATGTGCTTGTCGTCATCGTC |
| NO. 365 | |
| SEQ ID | CGAGGTGCTTCGTTAAACCGGCGGAGCCCAAGACAGAACAACCTTGTCGTCATCGTC |
| NO. 366 | |
| SEQ ID | CGAGGTGCTTCGTTAAACGGTCACAAACAGATCCATTGCGGTCTTGTCGTCATCGTC |
| NO. 367 | |
| SEQ ID | CGAGGTGCTTCGTTAAACAAACAGGTCCATAGCGGTAACATACTTGTCGTCATCGTC |
| NO. 368 | |
| SEQ ID | CGAGGTGCTTCGTTATGCGCCAACAATCCAGGTAACAACGTACTTGTCGTCATCGTC |
| NO. 369 | |
| SEQ ID | CGAGGTGCTTCGTTACAGAACCGGAACAGAACCATACAGAGCCTTGTCGTCATCGTC |
| NO. 370 | |
| SEQ ID | CGAGGTGCTTCGTTACAGCAGCAGAGACAGGGTTTCCAGCAGAGCCTTGTCGTCATCGTC |
| NO. 371 | |
| SEQ ID | CGAGGTGCTTCGTTACAGCAGAGACAGGGTTTCCAGCAGAGCCTTGTCGTCATCGTC |
| NO. 372 | |
| SEQ ID | CGAGGTGCTTCGTTAGATCCAGTAAAACATATTGAAGATCAGCTTGTCGTCATCGTC |
| NO. 373 | |
| SEQ ID | CGAGGTGCTTCGTTAAACCGGAGAGGTGGTCGGGTCCAGAGACTTGTCGTCATCGTC |
| NO. 374 | |
| SEQ ID | CGAGGTGCTTCGTTACAGGTAGATGTTAGCCAGAGACATTTTCTTGTCGTCATCGTC |
| NO. 375 | |
| SEQ ID | CGAGGTGCTTCGTTAAACCAGGAAGTCCAGCGGGAAAGAGAACTTGTCGTCATCGTC |
| NO. 376 | |
| SEQ ID | CGAGGTGCTTCGTTACAGCTTCACGGTATATTCTTGCAGAAACTTGTCGTCATCGTC |
| NO. 377 | |
| SEQ ID | CGAGGTGCTTCGTTAGATTTTGGTAATCATAGCGTTCAGGATCTTGTCGTCATCGTC |
| NO. 378 | |
| SEQ ID | CGAGGTGCTTCGTTAGATGTAAGCGTGCAGTTCAGACAGTTTCTTGTCGTCATCGTC |
| NO. 379 | |
| SEQ ID | CGAGGTGCTTCGTTAAACGCTAACCGGCAGTAACAGCAGAGACTTGTCGTCATCGTC |
| NO. 380 | |
| SEQ ID | CGAGGTGCTTCGTTACAGGGTCACGGTCAGTTTTGCCATATACTTGTCGTCATCGTC |
| NO. 381 | |
| SEQ ID | CGAGGTGCTTCGTTACAGTTCACCCGGAGAACCATACATATACTTGTCGTCATCGTC |
| NO. 382 | |
| SEQ ID | CGAGGTGCTTCGTTAAACAATGTAAACAATAGAGAACGGCATAACCTTGTCGTCATCGTC |
| NO. 383 | |
| SEQ ID | CGAGGTGCTTCGTTAGATGTAAACGATAGAGAACGGCATAACCTTGTCGTCATCGTC |
| NO. 384 | |
| SEQ ID | CGAGGTGCTTCGTTAGATGTAAACAATAGAGAACGGCATAACCAGCTTGTCGTCATCGTC |
| NO. 385 | |
| SEQ ID | CGAGGTGCTTCGTTACAGGTAGAACAGGTGAGAAGAAGACATGGTCTTGTCGTCATCGTC |
| NO. 386 | |
| SEQ ID | CGAGGTGCTTCGTTACAGCAGGATAGAAATGCCGGTAATGAACTTGTCGTCATCGTC |
| NO. 387 | |
| SEQ ID | CGAGGTGCTTCGTTAAACCAGGAATGCACGGTGCAGCAGAACCTTGTCGTCATCGTC |
| NO. 388 | |
| SEQ ID | CGAGGTGCTTCGTTAAACCAGGTTCAGAACTTCAGAAGAGAACTTGTCGTCATCGTC |
| NO. 389 | |
| SEQ ID | CGAGGTGCTTCGTTACAGAAACTCCAGGTACGGACCCAGACGCTTGTCGTCATCGTC |
| NO. 390 | |
| SEQ ID | CGAGGTGCTTCGTTAAACCGGCATAATTTCACGAGACAGTTTCTTGTCGTCATCGTC |
| NO. 391 | |
| SEQ ID | CGAGGTGCTTCGTTAAACATAGTACGGTAAGATTGCCAGCAGCTTGTCGTCATCGTC |
| NO. 392 | |
| SEQ ID | CGAGGTGCTTCGTTAAGCGTTTGCGTTCAGGTCCGGCAGAAACTTGTCGTCATCGTC |
| NO. 393 | |
| SEQ ID | CGAGGTGCTTCGTTAGATCGGAGAAGAGATTTCAGAGGTGTACTTGTCGTCATCGTC |
| NO. 394 | |
| SEQ ID | CGAGGTGCTTCGTTACAGAGCCGGATAGCGATTGAACAGGTTCTTGTCGTCATCGTC |
| NO. 395 | |
| SEQ ID | CGAGGTGCTTCGTTACAGCAGCCAGGTAACTGATGCGATCAGGAACTTGTCGTCATCGTC |
| NO. 396 | |
| SEQ ID | CGAGGTGCTTCGTTACAGCCAGGTAACAGATGCGATCAGGAACTTGTCGTCATCGTC |
| NO. 397 | |
| SEQ ID | CGAGGTGCTTCGTTAAACAGCTTCCATAAATTCGTCCAGGAACTTGTCGTCATCGTC |
| NO. 398 | |
| SEQ ID | CGAGGTGCTTCGTTAGATCAGCGGGATAACCGGAGACAGAGCCTTGTCGTCATCGTC |
| NO. 399 | |
| SEQ ID | CGAGGTGCTTCGTTAAGCCAGTTGAACGGCAGGCCATAAATACTTGTCGTCATCGTC |
| NO. 400 | |
| SEQ ID | CGAGGTGCTTCGTTAAACAACACGTAACGGTTCCCATAAACGCTTGTCGTCATCGTC |
| NO. 401 | |
| SEQ ID | CGAGGTGCTTCGTTACAGCAGGCACGGGGTCATACCGAACAGCAGCTTGTCGTCATCGTC |
| NO. 402 | |
| SEQ ID | CGAGGTGCTTCGTTACAGGCACGGGGTCATACCGAACAGCAGCTTGTCGTCATCGTC |
| NO. 403 | |
| SEQ ID | CGAGGTGCTTCGTTACAGTTTCGCAATGGTTTCGTCCAGACCCTTGTCGTCATCGTC |
| NO. 404 | |
| SEQ ID | CGAGGTGCTTCGTTAAACAGGCGGCGGCATACCAATAACACGCTTGTCGTCATCGTC |
| NO. 405 | |
| SEQ ID | CGAGGTGCTTCGTTAAACTTCCGGTTCTTTTTCGTCCAGCAGCTTGTCGTCATCGTC |
| NO. 406 | |
| SEQ ID | CGAGGTGCTTCGTTAGATAGAAGAGTAATACTGGTCAATGAACTTGTCGTCATCGTC |
| NO. 407 | |
| SEQ ID | CGAGGTGCTTCGTTATGCTTCGTAGTTCAGACCCGTCAGAATCTTGTCGTCATCGTC |
| NO. 408 | |
| SEQ ID | CGAGGTGCTTCGTTACAGGGTCGGGTCAGCAGGGTCCAGAATCTTGTCGTCATCGTC |
| NO. 409 | |
| SEQ ID | CGAGGTGCTTCGTTACAGGAACGGAACCATAACAATCAGGATCTTGTCGTCATCGTC |
| NO. 410 | |
| SEQ ID | CGAGGTGCTTCGTTACATCAGGGTAACCAGGTACAGCATGAACTTGTCGTCATCGTC |
| NO. 411 | |
| SEQ ID | CGAGGTGCTTCGTTAAACGGTAACCAGGTACATGAACAGGAACTTGTCGTCATCGTC |
| NO. 412 | |
| SEQ ID | CGAGGTGCTTCGTTACAGCAGCGGGAAAAACACGTTCAGGAACTTGTCGTCATCGTC |
| NO. 413 | |
| SEQ ID | CGAGGTGCTTCGTTACAGTGCCAGGTTACCCAGAAAAATATACTTGTCGTCATCGTC |
| NO. 414 | |
| SEQ ID | CGAGGTGCTTCGTTAGGTGGTATAGAACACAGCAACCATTTTCTTGTCGTCATCGTC |
| NO. 415 | |
| SEQ ID | CGAGGTGCTTCGTTACAGGGTGTAGATAAACGGATTCAGAACCTTGTCGTCATCGTC |
| NO. 416 | |
| SEQ ID | CGAGGTGCTTCGTTAAACAAACACAACCAGTTCGTTCACATACTTGTCGTCATCGTC |
| NO. 417 | |
| SEQ ID | CGAGGTGCTTCGTTAAACAACGGTCACGGTGTAGATACCCAGGAACTTGTCGTCATCGTC |
| NO. 418 | |
| SEQ ID | CGAGGTGCTTCGTTAAACGGTAACGGTGTAGATACCCAGGAACTTGTCGTCATCGTC |
| NO. 419 | |
| SEQ ID | CGAGGTGCTTCGTTAGATAGATGCGAAAAAGGTGAACAGGAACTTGTCGTCATCGTC |
| NO. 420 | |
| SEQ ID | CGAGGTGCTTCGTTAGATAGCCAGCAGATAGCAGTCAATAGACTTGTCGTCATCGTC |
| NO. 421 | |
| SEQ ID | CGAGGTGCTTCGTTACAGGTGCGGAGAACCTTGCAGCAGAGACTTGTCGTCATCGTC |
| NO. 422 | |
| SEQ ID | CGAGGTGCTTCGTTACAGCGGGAACAGACGTTCACCCAGCATCTTGTCGTCATCGTC |
| NO. 423 | |
| SEQ ID | CGAGGTGCTTCGTTAAACAAACAGCAGAACAGAGAACAGGAACTTGTCGTCATCGTC |
| NO. 424 | |
| SEQ ID | CGAGGTGCTTCGTTAAACGCCCATAACCAGCGGCAGAACCAGCTTGTCGTCATCGTC |
| NO. 425 | |
| SEQ ID | CGAGGTGCTTCGTTACAGCGGCAGAACCAGATCATGCAGACGCTTGTCGTCATCGTC |
| NO. 426 | |
| SEQ ID | CGAGGTGCTTCGTTAAACAGAGTAAACGTGAGAACCAACAGCCTTGTCGTCATCGTC |
| NO. 427 | |
| SEQ ID | CGAGGTGCTTCGTTATGCCGGAAAAGAGATAATGCTCAGCAGCTTGTCGTCATCGTC |
| NO. 428 | |
| SEQ ID | CGAGGTGCTTCGTTACATGAACGGAGAGAAAACGGTCAGGAACTTGTCGTCATCGTC |
| NO. 429 | |
| SEQ ID | CGAGGTGCTTCGTTATGCAGAAGAGAATGCAGCGAACAGAACCTTGTCGTCATCGTC |
| NO. 430 | |
| SEQ ID | CGAGGTGCTTCGTTACAGACCCCACAGAGAAACCAGTAACAGCTTGTCGTCATCGTC |
| NO. 431 | |
| SEQ ID | CGAGGTGCTTCGTTAAACTTCAACAACACCACCCAGTTGATGCTTGTCGTCATCGTC |
| NO. 432 | |
| SEQ ID | CGAGGTGCTTCGTTAAACACGTTGAACTGCATCCAGGATAGACTTGTCGTCATCGTC |
| NO. 433 | |
| SEQ ID | CGAGGTGCTTCGTTACAGAGAGTTGCGATATTCAGACAGTTTCTTGTCGTCATCGTC |
| NO. 434 | |
| SEQ ID | CGAGGTGCTTCGTTAAATGAATTTCAGGTTCTGGTCTGCTAACAGCTTGTCGTCATCGTC |
| NO. 435 | |
| SEQ ID | CGAGGTGCTTCGTTACAGGTACGGCTCAAAATAATTCAGAACCTTGTCGTCATCGTC |
| NO. 436 | |
| SEQ ID | CGAGGTGCTTCGTTAAATAGACGGAATTGCGCCAACCAGAGCCTTGTCGTCATCGTC |
| NO. 437 | |
| SEQ ID | CGAGGTGCTTCGTTAAACACGGGTCAGCGGGTTATACAGGAACTTGTCGTCATCGTC |
| NO. 438 | |
| SEQ ID | CGAGGTGCTTCGTTAAACCGGGGTAGAGATTTCAACCATGTGCTTGTCGTCATCGTC |
| NO. 439 | |
| SEQ ID | CGAGGTGCTTCGTTAAACAATTTCGTCACCAGCCAGCAGTTTCTTGTCGTCATCGTC |
| NO. 440 | |
| SEQ ID | CGAGGTGCTTCGTTAAACGGTGTGAACAACCTGACCACCTAAAATCTTGTCGTCATCGTC |
| NO. 441 | |
| SEQ ID | CGAGGTGCTTCGTTACAGAAAAACAGGGCTACCCGCCATTGCCTTGTCGTCATCGTC |
| NO. 442 | |
| SEQ ID | CGAGGTGCTTCGTTAAGCTGCAATGATGGTGGTGCTCATGTACTTGTCGTCATCGTC |
| NO. 443 | |
| SEQ ID | CGAGGTGCTTCGTTACAGACCAAAAATCTGAGCAACCAGGATCTTGTCGTCATCGTC |
| NO. 444 | |
| SEQ ID | CGAGGTGCTTCGTTACAGACCCAGAACCTGTGCAATCAGAATCTTGTCGTCATCGTC |
| NO. 445 | |
| SEQ ID | CGAGGTGCTTCGTTACAGACCCAGGAACAGAGCAGCCCAGATACGCTTGTCGTCATCGTC |
| NO. 446 | |
| SEQ ID | CGAGGTGCTTCGTTACAGCAGAACCGTCCAACCACCCAGCAGCTTGTCGTCATCGTC |
| NO. 447 | |
| SEQ ID | CGAGGTGCTTCGTTAAACGCCATGAACCAGGAACTCAAACAGTTTCTTGTCGTCATCGTC |
| NO. 448 | |
| SEQ ID | CGAGGTGCTTCGTTAGGTGTACGGCAGCGGTTTAGCCAGTTTCTTGTCGTCATCGTC |
| NO. 449 | |
| SEQ ID | CGAGGTGCTTCGTTACAGCAGAACCGGCTGGTGAGACAGTTTCTTGTCGTCATCGTC |
| NO. 450 | |
| SEQ ID | CGAGGTGCTTCGTTACAGACCGTTTGCTTCGTCCAGCAGGAACTTGTCGTCATCGTC |
| NO. 451 | |
| SEQ ID | CGAGGTGCTTCGTTAGGTGGTAGCCAGAGAGTCTTGCAGATACTTGTCGTCATCGTC |
| NO. 452 | |
| SEQ ID | CGAGGTGCTTCGTTACAGAGAAGAAGAGATAGATGCCATCAGGAACTTGTCGTCATCGTC |
| NO. 453 | |
| SEQ ID | CGAGGTGCTTCGTTAAGAAGAAGAGATAGATGCCATCAGGAACTTGTCGTCATCGTC |
| NO. 454 | |
| SEQ ID | CGAGGTGCTTCGTTACAGGCTGCTTGAAATAGATGCCATCAGCTTGTCGTCATCGTC |
| NO. 455 | |
| SEQ ID | CGAGGTGCTTCGTTATGCCGAGAAGTACAGAGCGAACAGCAGCTTGTCGTCATCGTC |
| NO. 456 | |
| SEQ ID | CGAGGTGCTTCGTTAAATACCCGGATAGTGTTTCATCAGACGCTTGTCGTCATCGTC |
| NO. 457 | |
| SEQ ID | CGAGGTGCTTCGTTACAGAACACCAGAATATGCTGACATGAACTTGTCGTCATCGTC |
| NO. 458 | |
| SEQ ID | CGAGGTGCTTCGTTAAACGGTTGCCAGCAGCGGACCCATACCCTTGTCGTCATCGTC |
| NO. 459 | |
| SEQ ID | CGAGGTGCTTCGTTACAGCAGGATGGTGAAGTTTTCCAGAACAGACTTGTCGTCATCGTC |
| NO. 460 | |
| SEQ ID | CGAGGTGCTTCGTTAAACCAGCAGGATGGTGAAGTTTTCCAGAACCTTGTCGTCATCGTC |
| NO. 461 | |
| SEQ ID | CGAGGTGCTTCGTTACATTTTGGTTTCCAGGGTCATCAGGAACTTGTCGTCATCGTC |
| NO. 462 | |
| SEQ ID | CGAGGTGCTTCGTTAAACCAGGTAGAAAGAAACTGCAAACGTAACCTTGTCGTCATCGTC |
| NO. 463 | |
| SEQ ID | CGAGGTGCTTCGTTACAGCAGTAAGGTAACCTGAGACAGAGCCTTGTCGTCATCGTC |
| NO. 464 | |
| SEQ ID | CGAGGTGCTTCGTTAAACAGATGCAGCGTGTTCCGGGGTGTACTTGTCGTCATCGTC |
| NO. 465 | |
| SEQ ID | CGAGGTGCTTCGTTAGGTTTCCCAGAAGGTTTCAGCCAGAGACTTGTCGTCATCGTC |
| NO. 466 | |
| SEQ ID | CGAGGTGCTTCGTTAAACGGTGTTAGAGATAGCAGCCATACGCTTGTCGTCATCGTC |
| NO. 467 | |
| SEQ ID | CGAGGTGCTTCGTTACAGCGGAACAGACGGAGAAGCCAGAACCTTGTCGTCATCGTC |
| NO. 468 | |
| SEQ ID | CGAGGTGCTTCGTTAAACAGACGGAGATGCCAGAACCATGTACTTGTCGTCATCGTC |
| NO. 469 | |
| SEQ ID | CGAGGTGCTTCGTTACAGAGACACGTCCTGTTTCAGCAGCATCTTGTCGTCATCGTC |
| NO. 470 | |
| SEQ ID | CGAGGTGCTTCGTTACAGTGCAATTAACATATTCAGCAGTAACTTGTCGTCATCGTC |
| NO. 471 | |
| SEQ ID | CGAGGTGCTTCGTTACAGAGCAGATGCGTAACCGATCATAAACTTGTCGTCATCGTC |
| NO. 472 | |
| SEQ ID | CGAGGTGCTTCGTTATGCGTAACCAATCATAAACAGCAGGTACTTGTCGTCATCGTC |
| NO. 473 | |
| SEQ ID | CGAGGTGCTTCGTTAAACCGGGTTGATGTCCAGCGGCAGTTTCTTGTCGTCATCGTC |
| NO. 474 | |
| SEQ ID | CGAGGTGCTTCGTTAGATTTCAAATGACTGGTTCAGTTGTGACTTGTCGTCATCGTC |
| NO. 475 | |
| SEQ ID | CGAGGTGCTTCGTTAGATCAGGTGGATGCACGGGATAATCAGCTTGTCGTCATCGTC |
| NO. 476 | |
| SEQ ID | CGAGGTGCTTCGTTAGATTGAGCTACTTGCCCACAGCATTAACTTGTCGTCATCGTC |
| NO. 477 | |
| SEQ ID | CGAGGTGCTTCGTTAGATCAGAGACAGGTGTGAGATAATCATCTTGTCGTCATCGTC |
| NO. 478 | |
| SEQ ID | CGAGGTGCTTCGTTAAACAGAAACGTCCAGGTAGGTCAGGAACTTGTCGTCATCGTC |
| NO. 479 | |
| SEQ ID | CGAGGTGCTTCGTTAAACAGAAACATTGAAGGTCAGCAGTAACTTGTCGTCATCGTC |
| NO. 480 | |
| SEQ ID | CGAGGTGCTTCGTTAAACAAACGGGTTCATCCACAGCAGGCTCTTGTCGTCATCGTC |
| NO. 481 | |
| SEQ ID | CGAGGTGCTTCGTTAAACGTGATACCATTCTTCCTGGGTGAACTTGTCGTCATCGTC |
| NO. 482 | |
| SEQ ID | CGAGGTGCTTCGTTAAACAGAAATGTCCTGTGAAAACAGATTCTTGTCGTCATCGTC |
| NO. 483 | |
| SEQ ID NO. | |
| 484 | AAGCAGTGGTATCAACGCAGAGT XXXXXX TTT TTT TTT TTT TTT TTT TTT TTT TTT TTT VN |
| SEQ ID NO. | AAGCAGTGGTATCAACGCAGAGTCGACrGrG+G |
| 485 | |
| SEQ ID NO. | AAGCAGTGGTATCAACGCAGAGT |
| 486 | |
| SEQ ID NO. | CAAGCAGAAGACGGCATACGAGAT XXXXXXXX GTCTCGTGGGCTCGG |
| 487 | |
| SEQ ID NO. | AATGATACGGCGACCACCGAGATCTACACTCTTTCCCTACACGACGCTCTTCCGATCTNHNHNAAGCAGTGGTATC |
| 488 | AACGCAGAGT |
| SEQ ID NO. | AAGCAGTGGTATCAACGCAGAGT XXXXXX TTT TTT TTT TTT TTT TTT TTT TTT TTT TTT VN |
| 484 | |
| SEQ ID NO. | AAGCAGTGGTATCAACGCAGAGTCGACrGrG + G |
| 485 | |
| SEQ ID NO. | AAGCAGTGGTATCAACGCAGAGT |
| 486 | |
| SEQ ID NO. | CAAGCAGAAGACGGCATACGAGATXXXXXXXXGTCTCGTGGGCTCGG |
| 487 | |
| SEQ ID NO. | AATGATACGGCGACCACCGAGATCTACACTCTTTCCCTACACGACGCTCTTCCGATCTNHNHNAAGCAGTGGTATC |
| 488 | AACGCAGAGT |
| SEQ ID: 501 | ATGGACGACGACGACAAGCGTCAGTTCGGTCCGGACTGGATCGTTGCTTAACGAAGCACCTCGCTAAAAAAAAAA |
| | AAAAAAAAAAAAAAA |
| SEQ ID: 502 | ATGGACGACGACGACAAGATGGTTGGGGTCCGGACCCGCTGTACGTTTAACGAAGCACCTCGCTAAAAAAAAAA |
| | AAAAAAAAAAAAAAA |
| SEQ ID: 503 | ATGGACGACGACGACAAGAACCTGGCTCAGGACCTGGCTACCGTTTAACGAAGCACCTCGCTAAAAAAAAAAAAA |
| | AAAAAAAAAAAA |
| SEQ ID: 504 | ATGGACGACGACGACAAGCAGCTGGCTCGTCAGCAGGITCACGTTTAACGAAGCACCTCGCTAAAAAAAAAAAAA |
| | AAAAAAAAAAAA |
| SEQ ID: 505 | ATGGACGACGACGACAAGTTCCTGCAGGACGTTATGAACATCCTGTAACGAAGCACCTCGCTAAAAAAAAAAAAA |
| | AAAAAAAAAAAA |
| SEQ ID: 506 | ATGGACGACGACGACAAGCTGCTGCAGGAATACAACTGGGAACTGTAACGAAGCACCTCGCTAAAAAAAAAAAAA |
| | AAAAAAAAAAAA |
| SEQ ID: 507 | ATGGACGACGACGACAAGCGTATGATGGAATACGGTACCACCATGGTTTAACGAAGCACCTCGCTAAAAAAAAAA |
| | AAAAAAAAAAAAAAA |
| SEQ ID: 508 | ATGGACGACGACGACAAGGTTATGAACATCCTGCTGCAGTACGTTGTTTAACGAAGCACCTCGCTAAAAAAAAAAA |
| | AAAAAAAAAAAAAA |
| SEQ ID: 509 | ATGGACGACGACGACAAGGTTATGAACATCCTGCTGCAGTACGTTTAACGAAGCACCTCGCTAAAAAAAAAAAAAA |
| | AAAAAAAAAAA |
| SEQ ID: 510 | ATGGACGACGACGACAAGGAACTGGCTGAATACCTGTACAACATCTAACGAAGCACCTCGCTAAAAAAAAAAAAA |
| | AAAAAAAAAAAA |
| SEQ ID: 511 | ATGGACGACGACGACAAGATCCTGATGCACTGCCAGACCACCCTGTAACGAAGCACCTCGCTAAAAAAAAAAAAA |
| | AAAAAAAAAAAA |
| SEQ ID: 512 | ATGGACGACGACGACAAGATGCTGTACCAGCACCTGCTGCCGCTGTAACGAAGCACCTCGCTAAAAAAAAAAAAA |
| | AAAAAAAAAAAA |
| SEQ ID: 513 | ATGGACGACGACGACAAGGGTATCGTTGAACAGTGCTGCACCTCTATCTAACGAAGCACCTCGCTAAAAAAAAAAA |
| | AAAAAAAAAAAAAA |
| SEQ ID: 514 | ATGGACGACGACGACAAGGCTCTGTGGATGCGTCTGCTGCCGCTGCTGTAACGAAGCACCTCGCTAAAAAAAAAA |
| | AAAAAAAAAAAAAAA |
| SEQ ID: 515 | ATGGACGACGACGACAAGCTGGCTCTGTGGGGTCCGGACCCGGCTGCTTAACGAAGCACCTCGCTAAAAAAAAAA |
| | AAAAAAAAAAAAAAA |
| SEQ ID: 516 | ATGGACGACGACGACAAGCGTCTGCTGCCGCTGCTGGCTCTGCTGGCTCTGTAACGAAGCACCTCGCTAAAAAAAA |
| | AAAAAAAAAAAAAAAAA |
| SEQ ID: 517 | ATGGACGACGACGACAAGGCTCTGTGGATGCGTCTGCTGCCGCTGTAACGAAGCACCTCGCTAAAAAAAAAAAAA |
| | AAAAAAAAAAAA |
| SEQ ID: 518 | ATGGACGACGACGACAAGCACCTGGTTGAAGCTCTGTACCTGGTTTAACGAAGCACCTCGCTAAAAAAAAAAAAAA |
| | AAAAAAAAAAA |
| SEQ ID: 519 | ATGGACGACGACGACAAGTCTCTGCAGAAACGTGGTATCGTTGAACAGTAACGAAGCACCTCGCTAAAAAAAAAA |
| | AAAAAAAAAAAAAAA |
| SEQ ID: 520 | ATGGACGACGACGACAAGTCTCTGCAGCCGCTGGCTCTGGAAGGTTAACGAAGCACCTCGCTAAAAAAAAAAAAA |
| | AAAAAAAAAAAA |
| SEQ ID: 521 | ATGGACGACGACGACAAGTCTCTGTACCAGCTGGAAAACTACTGCTAACGAAGCACCTCGCTAAAAAAAAAAAAAA |
| | AAAAAAAAAAA |
| SEQ ID: 522 | ATGGACGACGACGACAAGGTTTGCGGTGAACGTGGTTTCTTCTACACCTAACGAAGCACCTCGCTAAAAAAAAAAA |
| | AAAAAAAAAAAAAA |
| SEQ ID: 523 | ATGGACGACGACGACAAGGCTCTGTGGGGTCCGGACCCGGCTGCTGCTTAACGAAGCACCTCGCTAAAAAAAAAA |
| | AAAAAAAAAAAAAAA |
| SEQ ID: 524 | ATGGACGACGACGACAAGCGTCTGCTGCCGCTGCTGGCTCTGCTGTAACGAAGCACCTCGCTAAAAAAAAAAAAA |
| | AAAAAAAAAAAA |
| SEQ ID: 525 | ATGGACGACGACGACAAGTGGGGTCCGGACCCGGCTGCTGCTTAACGAAGCACCTCGCTAAAAAAAAAAAAAAAA |
| | AAAAAAAAA |
| SEQ ID: 526 | ATGGACGACGACGACAAGTTCCTGATCGTTCTGTCTGTTGCTCTGTAACGAAGCACCTCGCTAAAAAAAAAAAAAA |
| | AAAAAAAAAAA |
| SEQ ID: 527 | ATGGACGACGACGACAAGAAACTGCAGGTTTTCCTGATCGTTCTGTAACGAAGCACCTCGCTAAAAAAAAAAAAAA |
| | AAAAAAAAAAA |
| SEQ ID: 528 | ATGGACGACGACGACAAGTTCCTGTGGTCTGTTTTCATGCTGATCTAACGAAGCACCTCGCTAAAAAAAAAAAAAA |
| | AAAAAAAAAAA |
| SEQ ID: 529 | ATGGACGACGACGACAAGTTCCTGTTCGCTGTTGGTTTCTACCTGTAACGAAGCACCTCGCTAAAAAAAAAAAAAA |
| | AAAAAAAAAAA |
| SEQ ID: 530 | ATGGACGACGACGACAAGCTGAACATCGACCTGCTGTGGTCTGTTTAACGAAGCACCTCGCTAAAAAAAAAAAAAA |
| | AAAAAAAAAAA |
| SEQ ID: 531 | ATGGACGACGACGACAAGGTTCTGTTCGGTCTGGGTTTCGCTATCTAACGAAGCACCTCGCTAAAAAAAAAAAAAA |
| | AAAAAAAAAAA |
| SEQ ID: 532 | ATGGACGACGACGACAAGTTCCTGTGGTCTGTTTTCTGGCTGATCTAACGAAGCACCTCGCTAAAAAAAAAAAAAA |
| | AAAAAAAAAAA |
| SEQ ID: 533 | ATGGACGACGACGACAAGAACCTGTTCCTGTTCCTGTTCGCTGTTTAACGAAGCACCTCGCTAAAAAAAAAAAAAA |
| | AAAAAAAAAAA |
| SEQ ID: 534 | ATGGACGACGACGACAAGTACCTGCTGCTGCGTGTTCTGAACATCTAACGAAGCACCTCGCTAAAAAAAAAAAAAA |
| | AAAAAAAAAAA |
| SEQ ID: 535 | ATGGACGACGACGACAAGCACCTGTGCGGTTCTCACCTGGTTGAAGCTTAACGAAGCACCTCGCTAAAAAAAAAAA |
| | AAAAAAAAAAAAAA |
| SEQ ID: 536 | ATGGACGACGACGACAAGTCTCACCTGGTTGAAGCTCTGTACCTGGTTTAACGAAGCACCTCGCTAAAAAAAAAAA |
| | AAAAAAAAAAAAAA |
| SEQ ID: 537 | ATGGACGACGACGACAAGCTGTGCGGTTCTCACCTGGTTGAAGCTCTGTAACGAAGCACCTCGCTAAAAAAAAAAA |
| | AAAAAAAAAAAAAA |
| SEQ ID: 538 | ATGGACGACGACGACAAGGCTCTGACCGCTGTTGCTGAAGAAGTTTAACGAAGCACCTCGCTAAAAAAAAAAAAA |
| | AAAAAAAAAAAA |
| SEQ ID: 539 | ATGGACGACGACGACAAGTCTCTGTACCACGTTTACGAAGTTAACCTGTAACGAAGCACCTCGCTAAAAAAAAAAA |
| | AAAAAAAAAAAAAA |
| SEQ ID: 540 | ATGGACGACGACGACAAGACCATCGCTGACTTCTGGCAGATGGTTTAACGAAGCACCTCGCTAAAAAAAAAAAAA |
| | AAAAAAAAAAAA |
| SEQ ID: 541 | ATGGACGACGACGACAAGGTTATCGTTATGCTGACCCCGCTGGTTTAACGAAGCACCTCGCTAAAAAAAAAAAAAA |
| | AAAAAAAAAAA |
| SEQ ID: 542 | ATGGACGACGACGACAAGCTGCTGCCGCCGCTGCTGGAACACCTGTAACGAAGCACCTCGCTAAAAAAAAAAAAA |
| | AAAAAAAAAAAA |
| SEQ ID: 543 | ATGGACGACGACGACAAGTCTCTGGCTGCTGGTGTTAAACTGCTGTAACGAAGCACCTCGCTAAAAAAAAAAAAAA |
| | AAAAAAAAAAA |
| SEQ ID: 544 | ATGGACGACGACGACAAGTCTCTGTCTCCGCTGCAGGCTGAACTGTAACGAAGCACCTCGCTAAAAAAAAAAAAAA |
| | AAAAAAAAAAA |
| SEQ ID: 545 | ATGGACGACGACGACAAGATGGTTTGGGAATCTGGTTGCACCGTTTAACGAAGCACCTCGCTAAAAAAAAAAAAA |
| | AAAAAAAAAAAA |
| SEQ ID: 546 | ATGGACGACGACGACAAGGTTATGATCATCGTTTCTTCTCTGGCTGTTTAACGAAGCACCTCGCTAAAAAAAAAAAA |
| | AAAAAAAAAAAAA |
| SEQ ID: 547 | ATGGACGACGACGACAAGGCTCTGGGTGACCTGTTCCAGTCTATCTAACGAAGCACCTCGCTAAAAAAAAAAAAAA |
| | AAAAAAAAAAA |
| SEQ ID: 548 | ATGGACGACGACGACAAGGACCTGACGTCTTTCCTGCTGTCTCTGTAACGAAGCACCTCGCTAAAAAAAAAAAAAA |
| | AAAAAAAAAAA |
| SEQ ID: 549 | ATGGACGACGACGACAAGGAAATCCTGGGTGCTCTGCTGTCTATCTAACGAAGCACCTCGCTAAAAAAAAAAAAAA |
| | AAAAAAAAAAA |
| SEQ ID: 550 | ATGGACGACGACGACAAGTTCCTGCTGTCTCTGTTCTCTCTGTGGCTGTAACGAAGCACCTCGCTAAAAAAAAAAAA |
| | AAAAAAAAAAAAA |
| SEQ ID: 551 | ATGGACGACGACGACAAGATCCTGGCTGTTGACGGTGTTCTGTCTGTTTAACGAAGCACCTCGCTAAAAAAAAAAA |
| | AAAAAAAAAAAAAA |
| SEQ ID: 552 | ATGGACGACGACGACAAGATCCTGGGTGCTCTGCTGTCTATCCTGTAACGAAGCACCTCGCTAAAAAAAAAAAAAA |
| | AAAAAAAAAAA |
| SEQ ID: 553 | ATGGACGACGACGACAAGATCCTGAAAGACTTCTCTATCCTGCTGTAACGAAGCACCTCGCTAAAAAAAAAAAAAA |
| | AAAAAAAAAAA |
| SEQ ID: 554 | ATGGACGACGACGACAAGATCCTGTCTGCTCACGTTGCTACCGCTTAACGAAGCACCTCGCTAAAAAAAAAAAAAA |
| | AAAAAAAAAAA |
| SEQ ID: 555 | ATGGACGACGACGACAAGCTGCTGATCGACCTGACCTCTTTCCTGTAACGAAGCACCTCGCTAAAAAAAAAAAAAA |
| | AAAAAAAAAAA |
| SEQ ID: 556 | ATGGACGACGACGACAAGCTGCTGATGGAAGGTGTTCCGAAATCTCTGTAACGAAGCACCTCGCTAAAAAAAAAA |
| | AAAAAAAAAAAAAAA |
| SEQ ID: 557 | ATGGACGACGACGACAAGICTATCTCTGTTCTGATCTCTGCTCTGTAACGAAGCACCTCGCTAAAAAAAAAAAAAAA |
| | AAAAAAAAAA |
| SEQ ID: 558 | ATGGACGACGACGACAAGTCTCTGAACTACTCTGGTGTTAAAGAACTGTAACGAAGCACCTCGCTAAAAAAAAAAA |
| | AAAAAAAAAAAAAA |
| SEQ ID: 559 | ATGGACGACGACGACAAGTCTGTTCACTCTCTGCACATCTGGTCTCTGTAACGAAGCACCTCGCTAAAAAAAAAAA |
| | AAAAAAAAAAAAAA |
| SEQ ID: 560 | ATGGACGACGACGACAAGGTTGTTACCGGTGTTCTGGTTTACCTGTAACGAAGCACCTCGCTAAAAAAAAAAAAAA |
| | AAAAAAAAAAA |
| SEQ ID: 561 | ATGGACGACGACGACAAGTTCATCTTCTCTATCCTGGTTCTGGCTTAACGAAGCACCTCGCTAAAAAAAAAAAAAAA |
| | AAAAAAAAAA |
| SEQ ID: 562 | ATGGACGACGACGACAAGATCCAGGCTACCGTTATGATCATCGTTTAACGAAGCACCTCGCTAAAAAAAAAAAAAA |
| | AAAAAAAAAAA |
| SEQ ID: 563 | ATGGACGACGACGACAAGAAAATGTACGCTTTCACCCTGGAATCTTAACGAAGCACCTCGCTAAAAAAAAAAAAAA |
| | AAAAAAAAAAA |
| SEQ ID: 564 | ATGGACGACGACGACAAGAAATCTCTGAACTACTCTGGTGTTAAATAACGAAGCACCTCGCTAAAAAAAAAAAAAA |
| | AAAAAAAAAAA |
| SEQ ID: 565 | ATGGACGACGACGACAAGCTGGCTGTTGACGGTGTTCTGTCTGTTTAACGAAGCACCTCGCTAAAAAAAAAAAAAA |
| | AAAAAAAAAAA |
| SEQ ID: 566 | ATGGACGACGACGACAAGCTGCTGTCTCTGTTCTCTCTGTGGCTGTAACGAAGCACCTCGCTAAAAAAAAAAAAAA |
| | AAAAAAAAAAA |
| SEQ ID: 567 | ATGGACGACGACGACAAGCGTCTGCTGTACCCGGACTACCAGATCTAACGAAGCACCTCGCTAAAAAAAAAAAAA |
| | AAAAAAAAAAAA |
| SEQ ID: 568 | ATGGACGACGACGACAAGACCATGCACTCTCTGACCATCCAGATGTAACGAAGCACCTCGCTAAAAAAAAAAAAAA |
| | AAAAAAAAAAA |
| SEQ ID: 569 | ATGGACGACGACGACAAGGTTGCTGCTAACATCGTTCTGACCGTTTAACGAAGCACCTCGCTAAAAAAAAAAAAAA |
| | AAAAAAAAAAA |
| SEQ ID: 570 | ATGGACGACGACGACAAGTGCCTGGGTCACAACCACAAAGAAGTTTAACGAAGCACCTCGCTAAAAAAAAAAAAA |
| | AAAAAAAAAAAA |
| SEQ ID: 571 | ATGGACGACGACGACAAGAAAATCGCTGACCCGATCTGCACCTTCATCTAACGAAGCACCTCGCTAAAAAAAAAAA |
| | AAAAAAAAAAAAAA |
| SEQ ID: 572 | ATGGACGACGACGACAAGAAAATGTACGCTTTCACCCTGGAATCTGTTTAACGAAGCACCTCGCTAAAAAAAAAAA |
| | AAAAAAAAAAAAAA |
| SEQ ID: 573 | ATGGACGACGACGACAAGCTGCTGATCGACCTGACCTCTTTCCTGCTGTAACGAAGCACCTCGCTAAAAAAAAAAA |
| | AAAAAAAAAAAAAA |
| SEQ ID: 574 | ATGGACGACGACGACAAGCTGCTGTCTATCCTGTGCATCTGGGTTTAACGAAGCACCTCGCTAAAAAAAAAAAAAA |
| | AAAAAAAAAAA |
| SEQ ID: 575 | ATGGACGACGACGACAAGTCTCTGTACAACACCGTTGCTACCCTGTACTAACGAAGCACCTCGCTAAAAAAAAAAA |
| | AAAAAAAAAAAAAA |
| SEQ ID: 576 | ATGGACGACGACGACAAGTTCCTGGGTAAAATCTGGCCGTCTTACAAATAACGAAGCACCTCGCTAAAAAAAAAAA |
| | AAAAAAAAAAAAAA |
| SEQ ID: 577 | ATGGACGACGACGACAAGCTGGTTGGTCCGACCCCGGTTAACATCTAACGAAGCACCTCGCTAAAAAAAAAAAAA |
| | AAAAAAAAAAAA |
| SEQ ID: 578 | ATGGACGACGACGACAAGGCTCTGGTTGAAATCTGCACCGAAATGTAACGAAGCACCTCGCTAAAAAAAAAAAAA |
| | AAAAAAAAAAAA |
| SEQ ID: 579 | ATGGACGACGACGACAAGGTTATCTACCAGTACATGGACGACCTGTAACGAAGCACCTCGCTAAAAAAAAAAAAA |
| | AAAAAAAAAAAA |
| SEQ ID: 580 | ATGGACGACGACGACAAGATCCTGAAAGAACCGGTTCACGGTGTTTAACGAAGCACCTCGCTAAAAAAAAAAAAA |
| | AAAAAAAAAAAA |
| SEQ ID: 581 | ATGGACGACGACGACAAGGCTATCATCCGTATCCTGCAGCAGCTGTAACGAAGCACCTCGCTAAAAAAAAAAAAA |
| | AAAAAAAAAAAA |
| SEQ ID: 582 | ATGGACGACGACGACAAGCGTGGTCCGGGTCGTGGTTTCGTTACCATCTAACGAAGCACCTCGCTAAAAAAAAAAA |
| | AAAAAAAAAAAAAA |
| SEQ ID: 583 | ATGGACGACGACGACAAGTCTCTGCTGAACGCTACCGACATCGCTGTTTAACGAAGCACCTCGCTAAAAAAAAAAA |
| | AAAAAAAAAAAAAA |
| SEQ ID: 584 | ATGGACGACGACGACAAGCTGCTGAACGCTACCGACATCGCTGTTTAACGAAGCACCTCGCTAAAAAAAAAAAAA |
| | AAAAAAAAAAAA |
| SEQ ID: 585 | ATGGACGACGACGACAAGCCGCTGACCTTCGGTTGGTGCTACAAACTGTAACGAAGCACCTCGCTAAAAAAAAAA |
| | AAAAAAAAAAAAAAA |
| SEQ ID: 586 | ATGGACGACGACGACAAGGTTCTGGAATGGCGTTTCGACTCTCGTCTGTAACGAAGCACCTCGCTAAAAAAAAAAA |
| | AAAAAAAAAAAAAA |
| SEQ ID: 587 | ATGGACGACGACGACAAGGGTATCCTGGGTTTCGTTTTCACCCTGTAACGAAGCACCTCGCTAAAAAAAAAAAAAA |
| | AAAAAAAAAAA |
| SEQ ID: 588 | ATGGACGACGACGACAAGAAACTGTACCAGAACCCGACCACCTACATCTAACGAAGCACCTCGCTAAAAAAAAAAA |
| | AAAAAAAAAAAAAA |
| SEQ ID: 589 | ATGGACGACGACGACAAGCGTCTGTACCAGAACCCGACCACCTACATCTAACGAAGCACCTCGCTAAAAAAAAAAA |
| | AAAAAAAAAAAAAA |
| SEQ ID: 590 | ATGGACGACGACGACAAGGCTATCATGGACAAAAACATCATCCTGTAACGAAGCACCTCGCTAAAAAAAAAAAAA |
| | AAAAAAAAAAAA |
| SEQ ID: 591 | ATGGACGACGACGACAAGTTCATGTACTCTGACTTCCACTTCATCTAACGAAGCACCTCGCTAAAAAAAAAAAAAA |
| | AAAAAAAAAAA |
| SEQ ID: 592 | ATGGACGACGACGACAAGAAACTGGTTGCTCTGGGTATCAACGCTGTTTAACGAAGCACCTCGCTAAAAAAAAAAA |
| | AAAAAAAAAAAAAA |
| SEQ ID: 593 | ATGGACGACGACGACAAGCTGCTGTTCAACATCCTGGGTGGTTGGGTTTAACGAAGCACCTCGCTAAAAAAAAAAA |
| | AAAAAAAAAAAAAA |
| SEQ ID: 594 | ATGGACGACGACGACAAGTGCATCAACGGTGTTTGCTGGACCGTTTAACGAAGCACCTCGCTAAAAAAAAAAAAA |
| | AAAAAAAAAAAA |
| SEQ ID: 595 | ATGGACGACGACGACAAGTACCTGCTGCCGCGTCGTGGTCCGCGTCTGTAACGAAGCACCTCGCTAAAAAAAAAA |
| | AAAAAAAAAAAAAAA |
| SEQ ID: 596 | ATGGACGACGACGACAAGTACCTGGTTGCTCTGGGTATCAACGCTGTTTAACGAAGCACCTCGCTAAAAAAAAAAA |
| | AAAAAAAAAAAAAA |
| SEQ ID: 597 | ATGGACGACGACGACAAGTACCTGGTTGCTCTGGGTGTTAACGCTGTTTAACGAAGCACCTCGCTAAAAAAAAAAA |
| | AAAAAAAAAAAAAA |
| SEQ ID: 598 | ATGGACGACGACGACAAGAAACTGGTTGCTCTGGGTATCAACAACGTTTAACGAAGCACCTCGCTAAAAAAAAAA |
| | AAAAAAAAAAAAAAA |
| SEQ ID: 599 | ATGGACGACGACGACAAGTCTCTGGTTGCTCTGGGTATCAACGCTGTTTAACGAAGCACCTCGCTAAAAAAAAAAA |
| | AAAAAAAAAAAAAA |
| SEQ ID: 600 | ATGGACGACGACGACAAGAAAATCGTTGCTCTGGGTATCAACGCTGTTTAACGAAGCACCTCGCTAAAAAAAAAAA |
| | AAAAAAAAAAAAAA |
| SEQ ID: 601 | ATGGACGACGACGACAAGTGCCTGGGTGGTCTGCTGACCATGGTTTAACGAAGCACCTCGCTAAAAAAAAAAAAA |
| | AAAAAAAAAAAA |
| SEQ ID: 602 | ATGGACGACGACGACAAGTACCTGCAGCAGAACTGGTGGACCCTGTAACGAAGCACCTCGCTAAAAAAAAAAAAA |
| | AAAAAAAAAAAA |
| SEQ ID: 603 | ATGGACGACGACGACAAGTACCTGCTGGAAATGCTGTGGCGTCTGTAACGAAGCACCTCGCTAAAAAAAAAAAAA |
| | AAAAAAAAAAAA |
| SEQ ID: 604 | ATGGACGACGACGACAAGTACGTTCTGGACCACCTGATCGTTGTTTAACGAAGCACCTCGCTAAAAAAAAAAAAAA |
| | AAAAAAAAAAA |
| SEQ ID: 605 | ATGGACGACGACGACAAGGGICTGTGCACCCTGGTTGCTATGCTGTAACGAAGCACCTCGCTAAAAAAAAAAAAA |
| | AAAAAAAAAAAA |
| SEQ ID: 606 | ATGGACGACGACGACAAGTACCTGCTGCCGGGTTGGAAACTGTAACGAAGCACCTCGCTAAAAAAAAAAAAAAAA |
| | AAAAAAAAA |
| SEQ ID: 607 | ATGGACGACGACGACAAGTCTCTGATCTCTGGTATGTGGCTGCTGTAACGAAGCACCTCGCTAAAAAAAAAAAAAA |
| | AAAAAAAAAAA |
| SEQ ID: 608 | ATGGACGACGACGACAAGACCCTGCTGGCTAACGTTACCGCTGTTTAACGAAGCACCTCGCTAAAAAAAAAAAAAA |
| | AAAAAAAAAAA |
| SEQ ID: 609 | ATGGACGACGACGACAAGTTCCTGTACGCTCTGGCTCTGCTGCTGTAACGAAGCACCTCGCTAAAAAAAAAAAAAA |
| | AAAAAAAAAAA |
| SEQ ID: 610 | ATGGACGACGACGACAAGGAAGTTAAAGAAAAACACGAATTCCTGTAACGAAGCACCTCGCTAAAAAAAAAAAAA |
| | AAAAAAAAAAAA |
| SEQ ID: 611 | ATGGACGACGACGACAAGATCCTGATGAACGACCAGGAAGTTGGTGTTTAACGAAGCACCTCGCTAAAAAAAAAA |
| | AAAAAAAAAAAAAAA |
| SEQ ID: 612 | ATGGACGACGACGACAAGGGTATCATCTACATCATCTACAAACTGTAACGAAGCACCTCGCTAAAAAAAAAAAAAA |
| | AAAAAAAAAAA |
| SEQ ID: 613 | ATGGACGACGACGACAAGGAAGCTGCTGGTATCGGTATCCTGACCGTTTAACGAAGCACCTCGCTAAAAAAAAAA |
| | AAAAAAAAAAAAAAA |
| SEQ ID: 614 | ATGGACGACGACGACAAGGAACTGGCTGGTATCGGTATCCTGACCGTTTAACGAAGCACCTCGCTAAAAAAAAAA |
| | AAAAAAAAAAAAAAA |
| SEQ ID: 615 | ATGGACGACGACGACAAGGCTCTGGCTGGTATCGGTATCCTGACCGTTTAACGAAGCACCTCGCTAAAAAAAAAAA |
| | AAAAAAAAAAAAAA |
| SEQ ID: 616 | ATGGACGACGACGACAAGGCTGCTGGTATCGGTATCCTGACCGTTTAACGAAGCACCTCGCTAAAAAAAAAAAAA |
| | AAAAAAAAAAAA |
| SEQ ID: 617 | ATGGACGACGACGACAAGGCTCTGGGTATCGGTATCCTGACCGTTTAACGAAGCACCTCGCTAAAAAAAAAAAAA |
| | AAAAAAAAAAAA |
| SEQ ID: 618 | ATGGACGACGACGACAAGCTGCTGGCTGGTATCGGTACCGTTCCGATCTAACGAAGCACCTCGCTAAAAAAAAAAA |
| | AAAAAAAAAAAAAA |
| SEQ ID: 619 | ATGGACGACGACGACAAGTGCACCTCTATCTGCTCTCTGTACTAACGAAGCACCTCGCTAAAAAAAAAAAAAAAAA |
| | AAAAAAAA |
| SEQ ID: 620 | ATGGACGACGACGACAAGTGCGGTTCTCACCTGGTTGAAGCTCTGTACTAACGAAGCACCTCGCTAAAAAAAAAAA |
| | AAAAAAAAAAAAAA |
| SEQ ID: 621 | ATGGACGACGACGACAAGGGTTCTCACCTGGTTGAAGCTCTGTACTAACGAAGCACCTCGCTAAAAAAAAAAAAAA |
| | AAAAAAAAAAA |
| SEQ ID: 622 | ATGGACGACGACGACAAGTGCCTGGAACTGGCTGAATACCTGTACTAACGAAGCACCTCGCTAAAAAAAAAAAAA |
| | AAAAAAAAAAAA |
| SEQ ID: 623 | ATGGACGACGACGACAAGTCTACCGCTAACACCAACATGTTCACCTACTAACGAAGCACCTCGCTAAAAAAAAAAA |
| | AAAAAAAAAAAAAA |
| SEQ ID: 624 | ATGGACGACGACGACAAGAAATGCCTGGAACTGGCTGAATACCTGTACTAACGAAGCACCTCGCTAAAAAAAAAA |
| | AAAAAAAAAAAAAAA |
| SEQ ID: 625 | ATGGACGACGACGACAAGCAGCAGGACAAACACTACGACCTGTCTTACTAACGAAGCACCTCGCTAAAAAAAAAA |
| | AAAAAAAAAAAAAAA |
| SEQ ID: 626 | ATGGACGACGACGACAAGGTTTCTGCTACCGCTGGTACCACCGTTTACTAACGAAGCACCTCGCTAAAAAAAAAAA |
| | AAAAAAAAAAAAAA |
| SEQ ID: 627 | ATGGACGACGACGACAAGTCTACCAAAGTTATCGACTTCCACTACTAACGAAGCACCTCGCTAAAAAAAAAAAAAA |
| | AAAAAAAAAAA |
| SEQ ID: 628 | ATGGACGACGACGACAAGTACCTGGCTTGCGAACGTCTGCTGTACTAACGAAGCACCTCGCTAAAAAAAAAAAAA |
| | AAAAAAAAAAAA |
| SEQ ID: 629 | ATGGACGACGACGACAAGGITACCGACGCTGCTCACCTGCTGATCTAACGAAGCACCTCGCTAAAAAAAAAAAAAA |
| | AAAAAAAAAAA |
| SEQ ID: 630 | ATGGACGACGACGACAAGCCGACCGAAAAAGGTGCTAACGAATACTAACGAAGCACCTCGCTAAAAAAAAAAAAA |
| | AAAAAAAAAAAA |
| SEQ ID: 631 | ATGGACGACGACGACAAGCTGATCGACCTGACCTCTTTCCTGCTGTAACGAAGCACCTCGCTAAAAAAAAAAAAAA |
| | AAAAAAAAAAA |
| SEQ ID: 632 | ATGGACGACGACGACAAGAAACCGACCGAAAAAGGTGCTAACGAATACTAACGAAGCACCTCGCTAAAAAAAAAA |
| | AAAAAAAAAAAAAAA |
| SEQ ID: 633 | ATGGACGACGACGACAAGGTTGTTACCGACGCTGCTCACCTGCTGATCTAACGAAGCACCTCGCTAAAAAAAAAAA |
| | AAAAAAAAAAAAAA |
| SEQ ID: 634 | ATGGACGACGACGACAAGCTGACGTCTTTTCCTGCTGTCTCTGTTCTAACGAAGCACCTCGCTAAAAAAAAAAAAAAA |
| | AAAAAAAAAA |
| SEQ ID: 635 | ATGGACGACGACGACAAGTCTACCAACGTTGGTTCTAACACCTACTAACGAAGCACCTCGCTAAAAAAAAAAAAAA |
| | AAAAAAAAAAA |
| SEQ ID: 636 | ATGGACGACGACGACAAGTCTTCTACCAACGTTGGTTCTAACACCTACTAACGAAGCACCTCGCTAAAAAAAAAAA |
| | AAAAAAAAAAAAAA |
| SEQ ID: 637 | ATGGACGACGACGACAAGCTGACCTCTCTGACCATCCTGCAGCTGTACTAACGAAGCACCTCGCTAAAAAAAAAAA |
| | AAAAAAAAAAAAAA |
| SEQ ID: 638 | ATGGACGACGACGACAAGCCGACCCACGAAGAACACCTGTTCTACTAACGAAGCACCTCGCTAAAAAAAAAAAAA |
| | AAAAAAAAAAAA |
| SEQ ID: 639 | ATGGACGACGACGACAAGATCCCGACCCACGAAGAACACCTGTTCTACTAACGAAGCACCTCGCTAAAAAAAAAAA |
| | AAAAAAAAAAAAAA |
| SEQ ID: 640 | ATGGACGACGACGACAAGACCTCTCTGACCATCCTGCAGCTGTACTAACGAAGCACCTCGCTAAAAAAAAAAAAAA |
| | AAAAAAAAAAA |
| SEQ ID: 641 | ATGGACGACGACGACAAGTCTACCGGTCACATGATCCTGGCTTACTAACGAAGCACCTCGCTAAAAAAAAAAAAAA |
| | AAAAAAAAAAA |
| SEQ ID: 642 | ATGGACGACGACGACAAGTTCGGTGACCACCCGGGTCACTCTTACTAACGAAGCACCTCGCTAAAAAAAAAAAAAA |
| | AAAAAAAAAAA |
| SEQ ID: 643 | ATGGACGACGACGACAAGATCTCTACCGGTCACATGATCCTGGCTTACTAACGAAGCACCTCGCTAAAAAAAAAAA |
| | AAAAAAAAAAAAAA |
| SEQ ID: 644 | ATGGACGACGACGACAAGTTCCAGGACTCTGGTCTGCTGTACTAACGAAGCACCTCGCTAAAAAAAAAAAAAAAA |
| | AAAAAAAAA |
| SEQ ID: 645 | ATGGACGACGACGACAAGCAGCTGTTCCAGGACTCTGGTCTGCTGTACTAACGAAGCACCTCGCTAAAAAAAAAAA |
| | AAAAAAAAAAAAAA |
| SEQ ID: 646 | ATGGACGACGACGACAAGCTGTCTTGGCACGACGACCTGACCCAGTACTAACGAAGCACCTCGCTAAAAAAAAAA |
| | AAAAAAAAAAAAAAA |
| SEQ ID: 647 | ATGGACGACGACGACAAGTGGCCGGACGAAGGTGCTTCTCTGTACTAACGAAGCACCTCGCTAAAAAAAAAAAAA |
| | AAAAAAAAAAAA |
| SEQ ID: 648 | ATGGACGACGACGACAAGGCTCTGGACATCGAAATCGCTACCTACTAACGAAGCACCTCGCTAAAAAAAAAAAAA |
| | AAAAAAAAAAAA |
| SEQ ID: 649 | ATGGACGACGACGACAAGCTGGCTCTGGACATCGAAATCGCTACCTACTAACGAAGCACCTCGCTAAAAAAAAAAA |
| | AAAAAAAAAAAAAA |
| SEQ ID: 650 | ATGGACGACGACGACAAGGTTTGCGGTGAACGTGGTTTCTTCTACACCTAACGAAGCACCTCGCTAAAAAAAAAAA |
| | AAAAAAAAAAAAAA |
| SEQ ID: 651 | ATGGACGACGACGACAAGGGTGAACGTGGTTTCTTCTACACCTAACGAAGCACCTCGCTAAAAAAAAAAAAAAAA |
| | AAAAAAAAA |
| SEQ ID: 652 | ATGGACGACGACGACAAGCTGGTTTGCGGTGAACGTGGTTTCTTCTACTAACGAAGCACCTCGCTAAAAAAAAAAA |
| | AAAAAAAAAAAAAA |
| SEQ ID: 653 | ATGGACGACGACGACAAGGCTCTGTGGGGTCCGGACCCGGCTGCTGCTTTCTAACGAAGCACCTCGCTAAAAAAA |
| | AAAAAAAAAAAAAAAAAA |
| SEQ ID: 654 | ATGGACGACGACGACAAGTGCACCGAACTGAAACTGTCTGACTACTAACGAAGCACCTCGCTAAAAAAAAAAAAA |
| | AAAAAAAAAAAA |
| SEQ ID: 655 | ATGGACGACGACGACAAGCACTCTAACCTGAACGACGCTACCTACTAACGAAGCACCTCGCTAAAAAAAAAAAAAA |
| | AAAAAAAAAAA |
| SEQ ID: 656 | ATGGACGACGACGACAAGAAATCTTGCCTGCCGGCTTGCGTTTACTAACGAAGCACCTCGCTAAAAAAAAAAAAAA |
| | AAAAAAAAAAA |
| SEQ ID: 657 | ATGGACGACGACGACAAGCTGGTTTCTGACGGTGGTCCGAACCTGTACTAACGAAGCACCTCGCTAAAAAAAAAA |
| | AAAAAAAAAAAAAAA |
| SEQ ID: 658 | ATGGACGACGACGACAAGGTTTCTGACGGTGGTCCGAACCTGTACTAACGAAGCACCTCGCTAAAAAAAAAAAAA |
| | AAAAAAAAAAAA |
| SEQ ID: 659 | ATGGACGACGACGACAAGGCTCTGGCTTCTTGCATGGGTCTGATCTACTAACGAAGCACCTCGCTAAAAAAAAAAA |
| | AAAAAAAAAAAAAA |
| SEQ ID: 660 | ATGGACGACGACGACAAGGGTTCTGAAGAACTGCGTTCTCTGTACTAACGAAGCACCTCGCTAAAAAAAAAAAAA |
| | AAAAAAAAAAAA |
| SEQ ID: 661 | ATGGACGACGACGACAAGTTCCGTGACTACGTTGACCGTTTCTACTAACGAAGCACCTCGCTAAAAAAAAAAAAAA |
| | AAAAAAAAAAA |
| SEQ ID: 662 | ATGGACGACGACGACAAGCAGCGTCCGCTGGTTACCATCAAAATCTAACGAAGCACCTCGCTAAAAAAAAAAAAA |
| | AAAAAAAAAAAA |
| SEQ ID: 663 | ATGGACGACGACGACAAGATCTCTGAACGTATCCTGTCTACCTACTAACGAAGCACCTCGCTAAAAAAAAAAAAAA |
| | AAAAAAAAAAA |
| SEQ ID: 664 | ATGGACGACGACGACAAGCGTCGTGGTTGGGAAGTTCTGAAATACTAACGAAGCACCTCGCTAAAAAAAAAAAAA |
| | AAAAAAAAAAAA |
| SEQ ID: 665 | ATGGACGACGACGACAAGATGGCTCTGTGGATGCGTCTGCTGCCGCTGTAACGAAGCACCTCGCTAAAAAAAAAA |
| | AAAAAAAAAAAAAAA |
| SEQ ID: 666 | ATGGACGACGACGACAAGTGGATGCGTCTGCTGCCGCTGCTGGCTCTGTAACGAAGCACCTCGCTAAAAAAAAAA |
| | AAAAAAAAAAAAAAA |
| SEQ ID: 667 | ATGGACGACGACGACAAGTGGATGCGTCTGCTGCCGCTGCTGTAACGAAGCACCTCGCTAAAAAAAAAAAAAAAA |
| | AAAAAAAAA |
| SEQ ID: 668 | ATGGACGACGACGACAAGCTGTGGATGCGTCTGCTGCCGCTGTAACGAAGCACCTCGCTAAAAAAAAAAAAAAAA |
| | AAAAAAAAA |
| SEQ ID: 669 | ATGGACGACGACGACAAGTCTCTGCAGAAACGTGGTATCGTTTAACGAAGCACCTCGCTAAAAAAAAAAAAAAAA |
| | AAAAAAAAA |
| SEQ ID: 670 | ATGGACGACGACGACAAGATGGCTCTGTGGATGCGTCTGCTGTAACGAAGCACCTCGCTAAAAAAAAAAAAAAAA |
| | AAAAAAAAA |
| SEQ ID: 671 | ATGGACGACGACGACAAGATGATGATCGCTCGTTTCAAAATGTAACGAAGCACCTCGCTAAAAAAAAAAAAAAAA |
| | AAAAAAAAA |
| SEQ ID: 672 | ATGGACGACGACGACAAGATGATGATCGCTCGTTTCAAAATGTTCTAACGAAGCACCTCGCTAAAAAAAAAAAAAA |
| | AAAAAAAAAAA |
| SEQ ID: 673 | ATGGACGACGACGACAAGATGTCTCGTAAACACAAATGGAAACTGTAACGAAGCACCTCGCTAAAAAAAAAAAAA |
| | AAAAAAAAAAAA |
| SEQ ID: 674 | ATGGACGACGACGACAAGCTGATGTCTCGTAAACACAAATGGAAACTGTAACGAAGCACCTCGCTAAAAAAAAAA |
| | AAAAAAAAAAAAAAA |
| SEQ ID: 675 | ATGGACGACGACGACAAGTCTCTGAAAAAAGGTGCTGCTGCTCTGTAACGAAGCACCTCGCTAAAAAAAAAAAAA |
| | AAAAAAAAAAAA |
| SEQ ID: 676 | ATGGACGACGACGACAAGTTCTCTCTGAAAAAAGGTGCTGCTGCTCTGTAACGAAGCACCTCGCTAAAAAAAAAAA |
| | AAAAAAAAAAAAAA |
| SEQ ID: 677 | ATGGACGACGACGACAAGCACCCGCGTTACTTCAACCAGCTGTAACGAAGCACCTCGCTAAAAAAAAAAAAAAAA |
| | AAAAAAAAA |
| SEQ ID: 678 | ATGGACGACGACGACAAGCTGATGCACTGCCAGACCACCCTGTAACGAAGCACCTCGCTAAAAAAAAAAAAAAAA |
| | AAAAAAAAA |
| SEQ ID: 679 | ATGGACGACGACGACAAGGCTATGATGATCGCTCGTTTCAAAATGTTCTAACGAAGCACCTCGCTAAAAAAAAAAA |
| | AAAAAAAAAAAAAA |
| SEQ ID: 680 | ATGGACGACGACGACAAGATGTCTCGTCTGTCTAAAGTTGCTCCGGTTTAACGAAGCACCTCGCTAAAAAAAAAAA |
| | AAAAAAAAAAAAAA |
| SEQ ID: 681 | ATGGACGACGACGACAAGATGGCTGCTCTGCCGCGTCTGATCGGTTTCTAACGAAGCACCTCGCTAAAAAAAAAAA |
| | AAAAAAAAAAAAAA |
| SEQ ID: 682 | ATGGACGACGACGACAAGATGATCGCTCGTTTCAAAATGTTCTAACGAAGCACCTCGCTAAAAAAAAAAAAAAAAA |
| | AAAAAAAA |
| SEQ ID: 683 | ATGGACGACGACGACAAGACCCTGAAAAAAATGCGTGAAATCTAACGAAGCACCTCGCTAAAAAAAAAAAAAAAA |
| | AAAAAAAAA |
| SEQ ID: 684 | ATGGACGACGACGACAAGGAAGCTAAACAGAAAGGTTTCGTTCCGTTCTAACGAAGCACCTCGCTAAAAAAAAAA |
| | AAAAAAAAAAAAAAA |
| SEQ ID: 685 | ATGGACGACGACGACAAGCGTATGATGGAATACGGTACCACCATGTAACGAAGCACCTCGCTAAAAAAAAAAAAA |
| | AAAAAAAAAAAA |
| SEQ ID: 686 | ATGGACGACGACGACAAGGAAGTTAAAGAAAAAGGTATGGCTGCTCTGTAACGAAGCACCTCGCTAAAAAAAAAA |
| | AAAAAAAAAAAAAAA |
| SEQ ID: 687 | ATGGACGACGACGACAAGGAAGTTAAAGAAAAAGGTATGGCTGCTTAACGAAGCACCTCGCTAAAAAAAAAAAAA |
| | AAAAAAAAAAAA |
| SEQ ID: 688 | ATGGACGACGACGACAAGTACGCTATGATGATCGCTCGTTTCAAAATGTAACGAAGCACCTCGCTAAAAAAAAAAA |
| | AAAAAAAAAAAAAA |
| SEQ ID: 689 | ATGGACGACGACGACAAGAACCCGCACAAAATGATGGGTGTTCCGCTGTAACGAAGCACCTCGCTAAAAAAAAAA |
| | AAAAAAAAAAAAAAA |
| SEQ ID: 690 | ATGGACGACGACGACAAGTCTCGTAAACACAAATGGAAACTGTAACGAAGCACCTCGCTAAAAAAAAAAAAAAAA |
| | AAAAAAAAA |
| SEQ ID: 691 | ATGGACGACGACGACAAGTTCCAGCAGGACAAACACTACGACCTGTAACGAAGCACCTCGCTAAAAAAAAAAAAA |
| | AAAAAAAAAAAA |
| SEQ ID: 692 | ATGGACGACGACGACAAGTACGCTTTCCTGCACGCTACCGACCTGTAACGAAGCACCTCGCTAAAAAAAAAAAAAA |
| | AAAAAAAAAAA |
| SEQ ID: 693 | ATGGACGACGACGACAAGTTCTCTCTGAAAAAAGGTGCTGCTGCTTAACGAAGCACCTCGCTAAAAAAAAAAAAAA |
| | AAAAAAAAAAA |
| SEQ ID: 694 | ATGGACGACGACGACAAGTCTCTGAAAAAAGGTGCTGCTGCTTAACGAAGCACCTCGCTAAAAAAAAAAAAAAAA |
| | AAAAAAAAA |
| SEQ ID: 695 | ATGGACGACGACGACAAGACCCTGAAAAAAATGCGTGAAATCATCTAACGAAGCACCTCGCTAAAAAAAAAAAAA |
| | AAAAAAAAAAAA |
| SEQ ID: 696 | ATGGACGACGACGACAAGGAACGTATGTCTCGTCTGTCTAAAGTTTAACGAAGCACCTCGCTAAAAAAAAAAAAAA |
| | AAAAAAAAAAA |
| SEQ ID: 697 | ATGGACGACGACGACAAGTACGCTAAATGGAAACTGTGCTCTGCTTAACGAAGCACCTCGCTAAAAAAAAAAAAA |
| | AAAAAAAAAAAA |
| SEQ ID: 698 | ATGGACGACGACGACAAGGCTGCTAAAATGTACGCTTTCACCCTGTAACGAAGCACCTCGCTAAAAAAAAAAAAAA |
| | AAAAAAAAAAA |
| SEQ ID: 699 | ATGGACGACGACGACAAGTGCCCGCGTGAACGTCCGGAAGAACTGTAACGAAGCACCTCGCTAAAAAAAAAAAAA |
| | AAAAAAAAAAAA |
| SEQ ID: 700 | ATGGACGACGACGACAAGTACGCTTACGCTAAATGGAAACTGTAACGAAGCACCTCGCTAAAAAAAAAAAAAAAA |
| | AAAAAAAAA |
| SEQ ID: 701 | ATGGACGACGACGACAAGTTCCTGCTGTCTCTGTTCTCTCTGTAACGAAGCACCTCGCTAAAAAAAAAAAAAAAAA |
| | AAAAAAAA |
| SEQ ID: 702 | ATGGACGACGACGACAAGTCTGTTCGTGCTGCTTTCGTTCACGCTCTGTAACGAAGCACCTCGCTAAAAAAAAAAA |
| | AAAAAAAAAAAAAA |
| SEQ ID: 703 | ATGGACGACGACGACAAGAACGCTTCTGTTCGTGCTGCTTTCTAACGAAGCACCTCGCTAAAAAAAAAAAAAAAAA |
| | AAAAAAAA |
| SEQ ID: 704 | ATGGACGACGACGACAAGCACTCTCTGCACATCTGGTCTCTGTAACGAAGCACCTCGCTAAAAAAAAAAAAAAAAA |
| | AAAAAAAA |
| SEQ ID: 705 | ATGGACGACGACGACAAGGAAGTTCTGAAACGTGAACCGCTGTAACGAAGCACCTCGCTAAAAAAAAAAAAAAAA |
| | AAAAAAAAA |
| SEQ ID: 706 | ATGGACGACGACGACAAGCTGAACCACCTGAAAGCTACCCCGATCTAACGAAGCACCTCGCTAAAAAAAAAAAAA |
| | AAAAAAAAAAAA |
| SEQ ID: 707 | ATGGACGACGACGACAAGATCCTGAAACTGCAGGTTTTCCTGTAACGAAGCACCTCGCTAAAAAAAAAAAAAAAAA |
| | AAAAAAAA |
| SEQ ID: 708 | ATGGACGACGACGACAAGATGGGTATCCTGAAACTGCAGGTTTTCTAACGAAGCACCTCGCTAAAAAAAAAAAAA |
| | AAAAAAAAAAAA |
| SEQ ID: 709 | ATGGACGACGACGACAAGATGGGTATCCTGAAACTGCAGGTTTTCCTGTAACGAAGCACCTCGCTAAAAAAAAAAA |
| | AAAAAAAAAAAAAA |
| SEQ ID: 710 | ATGGACGACGACGACAAGAACACCTACGGTAAACGTAACGCTGTTTAACGAAGCACCTCGCTAAAAAAAAAAAAA |
| | AAAAAAAAAAAA |
| SEQ ID: 711 | ATGGACGACGACGACAAGTTCCTGCACCGTAACGGTGTTCTGTAACGAAGCACCTCGCTAAAAAAAAAAAAAAAA |
| | AAAAAAAAA |
| SEQ ID: 712 | ATGGACGACGACGACAAGTACCTGAAAACCAACCTGTTCCTGTAACGAAGCACCTCGCTAAAAAAAAAAAAAAAAA |
| | AAAAAAAA |
| SEQ ID: 713 | ATGGACGACGACGACAAGAACCTGATCTTCAAATGGATCCTGTAACGAAGCACCTCGCTAAAAAAAAAAAAAAAA |
| | AAAAAAAAA |
| SEQ ID: 714 | ATGGACGACGACGACAAGTACGTTATGGTTACCGCTGCTCTGTAACGAAGCACCTCGCTAAAAAAAAAAAAAAAAA |
| | AAAAAAAA |
| SEQ ID: 715 | ATGGACGACGACGACAAGACCCTGTCTTTCCGTCTGCTGTGCGCTCTGTAACGAAGCACCTCGCTAAAAAAAAAAA |
| | AAAAAAAAAAAAAA |
| SEQ ID: 716 | ATGGACGACGACGACAAGTACCTGAAAACCAACCTGTTCCTGTTCCTGTAACGAAGCACCTCGCTAAAAAAAAAAA |
| | AAAAAAAAAAAAAA |
| SEQ ID: 717 | ATGGACGACGACGACAAGTACCTGAAAACCAACCTGTTCCTGTTCTAACGAAGCACCTCGCTAAAAAAAAAAAAAA |
| | AAAAAAAAAAA |
| SEQ ID: 718 | ATGGACGACGACGACAAGTCTTTCCGTCTGCTGTGCGCTCTGTAACGAAGCACCTCGCTAAAAAAAAAAAAAAAAA |
| | AAAAAAAA |
| SEQ ID: 719 | ATGGACGACGACGACAAGACCCTGCACCGTCTGACCTGGTCTTTCTAACGAAGCACCTCGCTAAAAAAAAAAAAAA |
| | AAAAAAAAAAA |
| SEQ ID: 720 | ATGGACGACGACGACAAGTGCGGTATGGACAAATTCTCTATCACCCTGTAACGAAGCACCTCGCTAAAAAAAAAAA |
| | AAAAAAAAAAAAAA |
| SEQ ID: 721 | ATGGACGACGACGACAAGTGCGGTATGGACAAATTCTCTATCTAACGAAGCACCTCGCTAAAAAAAAAAAAAAAA |
| | AAAAAAAAA |
| SEQ ID: 722 | ATGGACGACGACGACAAGAACCTGATCTTCAAATGGAAATCTATCTAACGAAGCACCTCGCTAAAAAAAAAAAAAA |
| | AAAAAAAAAAA |
| SEQ ID: 723 | ATGGACGACGACGACAAGTGGCCGTGCAACGGTCGTATCCTGTGCCTGTAACGAAGCACCTCGCTAAAAAAAAAA |
| | AAAAAAAAAAAAAAA |
| SEQ ID: 724 | ATGGACGACGACGACAAGGTTCTGCTGGAAAAAAAATCTCCGCTGTAACGAAGCACCTCGCTAAAAAAAAAAAAA |
| | AAAAAAAAAAAA |
| SEQ ID: 725 | ATGGACGACGACGACAAGTTCCTGGTTCGTTCTTTCTACCTGTAACGAAGCACCTCGCTAAAAAAAAAAAAAAAAA |
| | AAAAAAAA |
| SEQ ID: 726 | ATGGACGACGACGACAAGCACCTGCGTAACCGTGACCGTCTGTAACGAAGCACCTCGCTAAAAAAAAAAAAAAAA |
| | AAAAAAAAA |
| SEQ ID: 727 | ATGGACGACGACGACAAGTCTCCGATGCGTTCTGTTCTGCTGTAACGAAGCACCTCGCTAAAAAAAAAAAAAAAAA |
| | AAAAAAAA |
| SEQ ID: 728 | ATGGACGACGACGACAAGGCTGCTCTGCAGCGTCTGGCTGCTGTTTAACGAAGCACCTCGCTAAAAAAAAAAAAA |
| | AAAAAAAAAAAA |
| SEQ ID: 729 | ATGGACGACGACGACAAGCTGCCGGCTCGTACCTCTCCGATGTAACGAAGCACCTCGCTAAAAAAAAAAAAAAAA |
| | AAAAAAAAA |
| SEQ ID: 730 | ATGGACGACGACGACAAGCTGCTGGAAAAAAAATCTCCGCTGTAACGAAGCACCTCGCTAAAAAAAAAAAAAAAA |
| | AAAAAAAAA |
| SEQ ID: 731 | ATGGACGACGACGACAAGGACAAAGAACGTCTGGCTGCTCTGTAACGAAGCACCTCGCTAAAAAAAAAAAAAAAA |
| | AAAAAAAAA |
| SEQ ID: 732 | ATGGACGACGACGACAAGCACGCTCGTATCAAACTGAAAGTTTAACGAAGCACCTCGCTAAAAAAAAAAAAAAAA |
| | AAAAAAAAA |
| SEQ ID: 733 | ATGGACGACGACGACAAGTACCGTGGTCGTTCTTGCCCGATCTAACGAAGCACCTCGCTAAAAAAAAAAAAAAAAA |
| | AAAAAAAA |
| SEQ ID: 734 | ATGGACGACGACGACAAGCAGCAGGACAAAGAACGTCTGGCTGCTCTGTAACGAAGCACCTCGCTAAAAAAAAAA |
| | AAAAAAAAAAAAAAA |
| SEQ ID: 735 | ATGGACGACGACGACAAGGAACTGCCGGCTCGTACCTCTCCGATGTAACGAAGCACCTCGCTAAAAAAAAAAAAA |
| | AAAAAAAAAAAA |
| SEQ ID: 736 | ATGGACGACGACGACAAGTGCTACCGTGGTCGTTCTTGCCCGATCTAACGAAGCACCTCGCTAAAAAAAAAAAAAA |
| | AAAAAAAAAAA |
| SEQ ID: 737 | ATGGACGACGACGACAAGCGTCCGCGTGACCGTTCTGGTCTGTAACGAAGCACCTCGCTAAAAAAAAAAAAAAAA |
| | AAAAAAAAA |
| SEQ ID: 738 | ATGGACGACGACGACAAGTCTCCGATGCGTTCTGTTCTGCTGACCCTGTAACGAAGCACCTCGCTAAAAAAAAAAA |
| | AAAAAAAAAAAAAA |
| SEQ ID: 739 | ATGGACGACGACGACAAGGCTCTGCAGCGTCTGGCTGCTGTTTAACGAAGCACCTCGCTAAAAAAAAAAAAAAAA |
| | AAAAAAAAA |
| SEQ ID: 740 | ATGGACGACGACGACAAGGCTGCTCTGCAGCGTCTGGCTGCTGTTCTGTAACGAAGCACCTCGCTAAAAAAAAAAA |
| | AAAAAAAAAAAAAA |
| SEQ ID: 741 | ATGGACGACGACGACAAGCACCTGCGTAACCGTGACCGTCTGGCTTAACGAAGCACCTCGCTAAAAAAAAAAAAA |
| | AAAAAAAAAAAA |
| SEQ ID: 742 | ATGGACGACGACGACAAGCTGGCTAAAGAATGGCAGGCTCTGTAACGAAGCACCTCGCTAAAAAAAAAAAAAAAA |
| | AAAAAAAAA |
| SEQ ID: 743 | ATGGACGACGACGACAAGCAGGACAAAGAACGTCTGGCTGCTCTGTAACGAAGCACCTCGCTAAAAAAAAAAAAA |
| | AAAAAAAAAAAA |
| SEQ ID: 744 | ATGGACGACGACGACAAGAACCTGCAGATCCGTGAAACCTCTCTGTAACGAAGCACCTCGCTAAAAAAAAAAAAA |
| | AAAAAAAAAAAA |
| SEQ ID: 745 | ATGGACGACGACGACAAGCTGCTGAACGTTAAACTGGCTCTGTAACGAAGCACCTCGCTAAAAAAAAAAAAAAAA |
| | AAAAAAAAA |
| SEQ ID: 746 | ATGGACGACGACGACAAGGAACTGCGTCTGCGTCTGGACCAGCTGTAACGAAGCACCTCGCTAAAAAAAAAAAAA |
| | AAAAAAAAAAAA |
| SEQ ID: 747 | ATGGACGACGACGACAAGATGGAACGTCGTCGTATCACCTCTGCTTAACGAAGCACCTCGCTAAAAAAAAAAAAAA |
| | AAAAAAAAAAA |
| SEQ ID: 748 | ATGGACGACGACGACAAGTGGTACCGTTCTAAATTCGCTGACCTGTAACGAAGCACCTCGCTAAAAAAAAAAAAAA |
| | AAAAAAAAAAA |
| SEQ ID: 749 | ATGGACGACGACGACAAGCACCTGAAACGTAACATCGTTGTTTAACGAAGCACCTCGCTAAAAAAAAAAAAAAAA |
| | AAAAAAAAA |
| SEQ ID: 750 | ATGGACGACGACGACAAGTACCGTCGTCAGCTGCAGTCTCTGTAACGAAGCACCTCGCTAAAAAAAAAAAAAAAA |
| | AAAAAAAAA |
| SEQ ID: 751 | ATGGACGACGACGACAAGTACCGTTCTAAATTCGCTGACCTGTAACGAAGCACCTCGCTAAAAAAAAAAAAAAAAA |
| | AAAAAAAA |
| SEQ ID: 752 | ATGGACGACGACGACAAGTCTAACCTGCAGATCCGTGAAACCTCTCTGTAACGAAGCACCTCGCTAAAAAAAAAAA |
| | AAAAAAAAAAAAAA |
| SEQ ID: 753 | ATGGACGACGACGACAAGGAACTGCGTGAACTGCGTCTGCGTCTGTAACGAAGCACCTCGCTAAAAAAAAAAAAA |
| | AAAAAAAAAAAA |
| SEQ ID: 754 | ATGGACGACGACGACAAGGACTACCGTCGTCAGCTGCAGTCTCTGTAACGAAGCACCTCGCTAAAAAAAAAAAAA |
| | AAAAAAAAAAAA |
| SEQ ID: 755 | ATGGACGACGACGACAAGTCTGCTGCTCGTCGTTCTTACGTTTAACGAAGCACCTCGCTAAAAAAAAAAAAAAAAA |
| | AAAAAAAA |
| SEQ ID: 756 | ATGGACGACGACGACAAGGAAGGTCACCTGAAACGTAACATCGTTTAACGAAGCACCTCGCTAAAAAAAAAAAAA |
| | AAAAAAAAAAAA |
| SEQ ID: 757 | ATGGACGACGACGACAAGATGGAACGTCGTCGTATCACCTCTGCTGCTTAACGAAGCACCTCGCTAAAAAAAAAAA |
| | AAAAAAAAAAAAAA |
| SEQ ID: 758 | ATGGACGACGACGACAAGCTGCGTCTGCGTCTGGACCAGCTGTAACGAAGCACCTCGCTAAAAAAAAAAAAAAAA |
| | AAAAAAAAA |
| SEQ ID: 759 | ATGGACGACGACGACAAGGACCTGGAACGTAAAATCGAATCTCTGTAACGAAGCACCTCGCTAAAAAAAAAAAAA |
| | AAAAAAAAAAAA |
| SEQ ID: 760 | ATGGACGACGACGACAAGCTGCAGATCCGTGAAACCTCTCTGTAACGAAGCACCTCGCTAAAAAAAAAAAAAAAA |
| | AAAAAAAAA |
| SEQ ID: 761 | ATGGACGACGACGACAAGCGTGAACTGCGTCTGCGTCTGGACCAGCTGTAACGAAGCACCTCGCTAAAAAAAAAA |
| | AAAAAAAAAAAAAAA |
| SEQ ID: 762 | ATGGACGACGACGACAAGCTGGCTCGTATGCCGCCGCCGCTGTAACGAAGCACCTCGCTAAAAAAAAAAAAAAAA |
| | AAAAAAAAA |
| SEQ ID: 763 | ATGGACGACGACGACAAGGAAATCCGTACCCAGTACGAAGCTATGTAACGAAGCACCTCGCTAAAAAAAAAAAAA |
| | AAAAAAAAAAAA |
| SEQ ID: 764 | ATGGACGACGACGACAAGGGTCCGGGTACCCGTCTGTCTCTGTAACGAAGCACCTCGCTAAAAAAAAAAAAAAAA |
| | AAAAAAAAA |
| SEQ ID: 765 | ATGGACGACGACGACAAGGCTGACCGTGGTCTGCTGCGTGACATCTAACGAAGCACCTCGCTAAAAAAAAAAAAA |
| | AAAAAAAAAAAA |
| SEQ ID: 766 | ATGGACGACGACGACAAGGCTCTGAAATGCAAAGGTTTCCACGTTTAACGAAGCACCTCGCTAAAAAAAAAAAAA |
| | AAAAAAAAAAAA |
| SEQ ID: 767 | ATGGACGACGACGACAAGGAACTGCGTTCTCGTTACTGGGCTATCTAACGAAGCACCTCGCTAAAAAAAAAAAAAA |
| | AAAAAAAAAAA |
| SEQ ID: 768 | ATGGACGACGACGACAAGATCCTGAAAGGTAAATTCCAGACCGCTTAACGAAGCACCTCGCTAAAAAAAAAAAAA |
| | AAAAAAAAAAAA |
| SEQ ID: 769 | ATGGACGACGACGACAAGCGTCCGATCATCCGTCCGGCTACCCTGTAACGAAGCACCTCGCTAAAAAAAAAAAAAA |
| | AAAAAAAAAAA |
| SEQ ID: 770 | ATGGACGACGACGACAAGGAACTGCGTTCTCTGTACAACACCGTTTAACGAAGCACCTCGCTAAAAAAAAAAAAAA |
| | AAAAAAAAAAA |
| SEQ ID: 771 | ATGGACGACGACGACAAGGAAATCTACAAACGTTGGATCATCTAACGAAGCACCTCGCTAAAAAAAAAAAAAAAA |
| | AAAAAAAAA |
| SEQ ID: 772 | ATGGACGACGACGACAAGCGTGTTAAAGAAAAATACCAGCACCTGTAACGAAGCACCTCGCTAAAAAAAAAAAAA |
| | AAAAAAAAAAAA |
| SEQ ID: 773 | ATGGACGACGACGACAAGTACCTGAAAGACCAGCAGCTGCTGTAACGAAGCACCTCGCTAAAAAAAAAAAAAAAA |
| | AAAAAAAAA |
| SEQ ID: 774 | ATGGACGACGACGACAAGTGGCCGACCGTTCGTGAACGTATGTAACGAAGCACCTCGCTAAAAAAAAAAAAAAAA |
| | AAAAAAAAA |
| SEQ ID: 775 | ATGGACGACGACGACAAGTTCCTGAAAGAAAAAGGTGGTCTGTAACGAAGCACCTCGCTAAAAAAAAAAAAAAAA |
| | AAAAAAAAA |
| SEQ ID: 776 | ATGGACGACGACGACAAGGGTCCGAAAGTTAAACAGTGGCCGCTGTAACGAAGCACCTCGCTAAAAAAAAAAAAA |
| | AAAAAAAAAAAA |
| SEQ ID: 777 | ATGGACGACGACGACAAGTTCCTGCGTGGTCGTGCTTACGGTCTGTAACGAAGCACCTCGCTAAAAAAAAAAAAAA |
| | AAAAAAAAAAA |
| SEQ ID: 778 | ATGGACGACGACGACAAGCGTGCTAAATTCAAACAGCTGCTGTAACGAAGCACCTCGCTAAAAAAAAAAAAAAAA |
| | AAAAAAAAA |
| SEQ ID: 779 | ATGGACGACGACGACAAGCAGGCTAAATG |
| | AAAAAAAAAAAA |
| SEQ ID: 780 | ATGGACGACGACGACAAGTGCCCGCTGTCTAAAATCCTGCTGTAACGAAGCACCTCGCTAAAAAAAAAAAAAAAAA |
| | AAAAAAAA |
| SEQ ID: 781 | ATGGACGACGACGACAAGtggtccgtcacgcaatAAAAAAAAAAAAAAAAAAAAAAA*A*A |
| SEQ ID: 782 | ATGGACGACGACGACAAGaggtgattgtgggataAAAAAAAAAAAAAAAAAAAAAAA*A*A |
| SEQ ID: 783 | ATGGACGACGACGACAAGagcggcgttgatacttAAAAAAAAAAAAAAAAAAAAAAA*A*A |
| SEQ ID: 784 | ATGGACGACGACGACAAGtaggtcgcgcttgcttAAAAAAAAAAAAAAAAAAAAAAA*A*A |
| SEQ ID: 785 | ATGGACGACGACGACAAGtgttgcaggttgctgtAAAAAAAAAAAAAAAAAAAAAAA*A*A |
| SEQ ID: 786 | ATGGACGACGACGACAAGgatgtgagttatgcagAAAAAAAAAAAAAAAAAAAAAAA*A*A |
| SEQ ID: 787 | ATGGACGACGACGACAAGaggtatcgcagtctggAAAAAAAAAAAAAAAAAAAAAAA*A*A |
| SEQ ID: 788 | ATGGACGACGACGACAAGtataatgggcgtctctAAAAAAAAAAAAAAAAAAAAAAA*A*A |
| SEQ ID: 789 | ATGGACGACGACGACAAGttcggcctggtgtaacAAAAAAAAAAAAAAAAAAAAAAA*A*A |
| SEQ ID: 790 | ATGGACGACGACGACAAGcctacgtatcgaagttAAAAAAAAAAAAAAAAAAAAAAA*A*A |
| SEQ ID: 791 | ATGGACGACGACGACAAGtctgccttgtatccgcAAAAAAAAAAAAAAAAAAAAAAA*A*A |
| SEQ ID: 792 | ATGGACGACGACGACAAGtgttgaccttcctcttAAAAAAAAAAAAAAAAAAAAAAA*A*A |
| SEQ ID: 793 | ATGGACGACGACGACAAGcctcatgcagtattgaAAAAAAAAAAAAAAAAAAAAAAA*A*A |
| SEQ ID: 794 | ATGGACGACGACGACAAGagtcatccacgcactcAAAAAAAAAAAAAAAAAAAAAAA*A*A |
| SEQ ID: 795 | ATGGACGACGACGACAAGaggttgtcgaattcccAAAAAAAAAAAAAAAAAAAAAAA*A*A |
| SEQ ID: 796 | ATGGACGACGACGACAAGtgcagaaaggtcatctAAAAAAAAAAAAAAAAAAAAAAA*A*A |
| SEQ ID: 797 | ATGGACGACGACGACAAGatttccggatcaatgcAAAAAAAAAAAAAAAAAAAAAAA*A*A |
| SEQ ID: 798 | ATGGACGACGACGACAAGgaatccgtactgattgAAAAAAAAAAAAAAAAAAAAAAA*A*A |
| SEQ ID: 799 | ATGGACGACGACGACAAGagagcgcagacattgcAAAAAAAAAAAAAAAAAAAAAAA*A*A |
| SEQ ID: 800 | ATGGACGACGACGACAAGtgtatgtctaccgagaAAAAAAAAAAAAAAAAAAAAAAA*A*A |
| SEQ ID: 801 | ATGGACGACGACGACAAGtgcttcctacgttcgtAAAAAAAAAAAAAAAAAAAAAAA*A*A |
| SEQ ID: 802 | ATGGACGACGACGACAAGtagtggggtaaaccatAAAAAAAAAAAAAAAAAAAAAAA*A*A |
| SEQ ID: 803 | ATGGACGACGACGACAAGcaaattttccatggcgAAAAAAAAAAAAAAAAAAAAAAA*A*A |
| SEQ ID: 804 | ATGGACGACGACGACAAGaaggccttcgtttcgaAAAAAAAAAAAAAAAAAAAAAAA*A*A |
| SEQ ID: 805 | ATGGACGACGACGACAAGgtcgagggagatatgcAAAAAAAAAAAAAAAAAAAAAAA*A*A |
| SEQ ID: 806 | ATGGACGACGACGACAAGctggacccagacatatAAAAAAAAAAAAAAAAAAAAAAA*A*A |
| SEQ ID: 807 | ATGGACGACGACGACAAGtagtcaagcactcggcAAAAAAAAAAAAAAAAAAAAAAA*A*A |
| SEQ ID: 808 | ATGGACGACGACGACAAGactaaggcggaaatctAAAAAAAAAAAAAAAAAAAAAAA*A*A |
| SEQ ID: 809 | ATGGACGACGACGACAAGtttagtgccggtgataAAAAAAAAAAAAAAAAAAAAAAA*A*A |
| SEQ ID: 810 | ATGGACGACGACGACAAGacttgcaacctaccggAAAAAAAAAAAAAAAAAAAAAAA*A*A |
| SEQ ID: 811 | ATGGACGACGACGACAAGtctacaacggacgtgaAAAAAAAAAAAAAAAAAAAAAAA*A*A |
| SEQ ID: 812 | ATGGACGACGACGACAAGagcaaaaccctacctaAAAAAAAAAAAAAAAAAAAAAAA*A*A |
| SEQ ID: 813 | ATGGACGACGACGACAAGttatcatcggtatgggAAAAAAAAAAAAAAAAAAAAAAA*A*A |
| SEQ ID: 814 | ATGGACGACGACGACAAGttctgcggatcgtcctAAAAAAAAAAAAAAAAAAAAAAA*A*A |
| SEQ ID: 815 | ATGGACGACGACGACAAGcctgcaaaggtatagcAAAAAAAAAAAAAAAAAAAAAAA*A*A |
| SEQ ID: 816 | ATGGACGACGACGACAAGagtactaagaagcgccAAAAAAAAAAAAAAAAAAAAAAA*A*A |
| SEQ ID: 817 | ATGGACGACGACGACAAGttggatacttgctgagAAAAAAAAAAAAAAAAAAAAAAA*A*A |
| SEQ ID: 818 | ATGGACGACGACGACAAGgtgtctccaaatcttcAAAAAAAAAAAAAAAAAAAAAAA*A*A |
| SEQ ID: 819 | ATGGACGACGACGACAAGgactctattacccaccAAAAAAAAAAAAAAAAAAAAAAA*A*A |
| SEQ ID: 820 | ATGGACGACGACGACAAGcagggattccaatatcAAAAAAAAAAAAAAAAAAAAAAA*A*A |
| SEQ ID: 821 | ATGGACGACGACGACAAGtatgcctagacaggttAAAAAAAAAAAAAAAAAAAAAAA*A*A |
| SEQ ID: 822 | ATGGACGACGACGACAAGagtagcattttcggtgAAAAAAAAAAAAAAAAAAAAAAA*A*A |
| SEQ ID: 823 | ATGGACGACGACGACAAGgacgtacgattgctacAAAAAAAAAAAAAAAAAAAAAAA*A*A |
| SEQ ID: 824 | ATGGACGACGACGACAAGgctcatgacatcgctaAAAAAAAAAAAAAAAAAAAAAAA*A*A |
| SEQ ID: 825 | ATGGACGACGACGACAAGgccttcaattctatggAAAAAAAAAAAAAAAAAAAAAAA*A*A |
| SEQ ID: 826 | ATGGACGACGACGACAAGctagtgttacaggtgcAAAAAAAAAAAAAAAAAAAAAAA*A*A |
| SEQ ID: 827 | ATGGACGACGACGACAAGccgagtgctctaaccaAAAAAAAAAAAAAAAAAAAAAAA*A*A |
| SEQ ID: 828 | ATGGACGACGACGACAAGatacgtcgtggcaacgAAAAAAAAAAAAAAAAAAAAAAA*A*A |
| SEQ ID: 829 | ATGGACGACGACGACAAGactgaggtccgatctaAAAAAAAAAAAAAAAAAAAAAAA*A*A |
| SEQ ID: 830 | ATGGACGACGACGACAAGttcgctcggaacatacAAAAAAAAAAAAAAAAAAAAAAA*A*A |
| SEQ ID: 831 | ATGGACGACGACGACAAGcaactcggtagttgagAAAAAAAAAAAAAAAAAAAAAAA*A*A |
| SEQ ID: 832 | ATGGACGACGACGACAAGtttgtttaggggttgcAAAAAAAAAAAAAAAAAAAAAAA*A*A |
| SEQ ID: 833 | ATGGACGACGACGACAAGaagcgcatttcgttctAAAAAAAAAAAAAAAAAAAAAAA*A*A |
| SEQ ID: 834 | ATGGACGACGACGACAAGcgagctccaactatcaAAAAAAAAAAAAAAAAAAAAAAA*A*A |
| SEQ ID: 835 | ATGGACGACGACGACAAGaatctggacggcttgtAAAAAAAAAAAAAAAAAAAAAAA*A*A |
| SEQ ID: 836 | ATGGACGACGACGACAAGcatttatgggtggtcaAAAAAAAAAAAAAAAAAAAAAAA*A*A |
| SEQ ID: 837 | ATGGACGACGACGACAAGattcctgataccagagAAAAAAAAAAAAAAAAAAAAAAA*A*A |
| SEQ ID: 838 | ATGGACGACGACGACAAGtgcaaatgcccaatacAAAAAAAAAAAAAAAAAAAAAAA*A*A |
| SEQ ID: 839 | ATGGACGACGACGACAAGtcattgttgggtaacgAAAAAAAAAAAAAAAAAAAAAAA*A*A |
| SEQ ID: 840 | ATGGACGACGACGACAAGcagtagccacgtgtgaAAAAAAAAAAAAAAAAAAAAAAA*A*A |
| SEQ ID: 841 | ATGGACGACGACGACAAGagaggatgggattactAAAAAAAAAAAAAAAAAAAAAAA*A*A |
| SEQ ID: 842 | ATGGACGACGACGACAAGctataagcgaaaccagAAAAAAAAAAAAAAAAAAAAAAA*A*A |
| SEQ ID: 843 | ATGGACGACGACGACAAGtgacgggctgtagtttAAAAAAAAAAAAAAAAAAAAAAA*A*A |
| SEQ ID: 844 | ATGGACGACGACGACAAGcctgtgtaagacgctgAAAAAAAAAAAAAAAAAAAAAAA*A*A |
| SEQ ID: 845 | ATGGACGACGACGACAAGtatggagacacaaacgAAAAAAAAAAAAAAAAAAAAAAA*A*A |
| SEQ ID: 846 | ATGGACGACGACGACAAGtacgaagggcagcataAAAAAAAAAAAAAAAAAAAAAAA*A*A |
| SEQ ID: 847 | ATGGACGACGACGACAAGggccgatatagcaagtAAAAAAAAAAAAAAAAAAAAAAA*A*A |
| SEQ ID: 848 | ATGGACGACGACGACAAGgagtggtcacacaggtAAAAAAAAAAAAAAAAAAAAAAA*A*A |
| SEQ ID: 849 | ATGGACGACGACGACAAGatatgattcacggtggAAAAAAAAAAAAAAAAAAAAAAA*A*A |
| SEQ ID: 850 | ATGGACGACGACGACAAGtgaccgagaccagagaAAAAAAAAAAAAAAAAAAAAAAA*A*A |
| SEQ ID: 851 | ATGGACGACGACGACAAGgctatcattgagcggaAAAAAAAAAAAAAAAAAAAAAAA*A*A |
| SEQ ID: 852 | ATGGACGACGACGACAAGtagtacgcaggttgatAAAAAAAAAAAAAAAAAAAAAAA*A*A |
| SEQ ID: 853 | ATGGACGACGACGACAAGtggatgtaacgcagcaAAAAAAAAAAAAAAAAAAAAAAA*A*A |
| SEQ ID: 854 | ATGGACGACGACGACAAGtcaactttgagggcacAAAAAAAAAAAAAAAAAAAAAAA*A*A |
| SEQ ID: 855 | ATGGACGACGACGACAAGctgaaaacctttgaggAAAAAAAAAAAAAAAAAAAAAAA*A*A |
| SEQ ID: 856 | ATGGACGACGACGACAAGaaggaaatagagctccAAAAAAAAAAAAAAAAAAAAAAA*A*A |
| SEQ ID: 857 | ATGGACGACGACGACAAGgtaaatcgccctggtaAAAAAAAAAAAAAAAAAAAAAAA*A*A |
| SEQ ID: 858 | ATGGACGACGACGACAAGgccttgtgaagcacgaAAAAAAAAAAAAAAAAAAAAAAA*A*A |
| SEQ ID: 859 | ATGGACGACGACGACAAGctattgaacaccgcagAAAAAAAAAAAAAAAAAAAAAAA*A*A |
| SEQ ID: 860 | ATGGACGACGACGACAAGtagtcccgagaccagaAAAAAAAAAAAAAAAAAAAAAAA*A*A |
| SEQ ID: 861 | ATGGACGACGACGACAAGtaccttcgaaagggccAAAAAAAAAAAAAAAAAAAAAAA*A*A |
| SEQ ID: 862 | ATGGACGACGACGACAAGaggggaaagatgtcagAAAAAAAAAAAAAAAAAAAAAAA*A*A |
| SEQ ID: 863 | ATGGACGACGACGACAAGcacacgagagaacaccAAAAAAAAAAAAAAAAAAAAAAA*A*A |
| SEQ ID: 864 | ATGGACGACGACGACAAGgagaacaaacgtggcgAAAAAAAAAAAAAAAAAAAAAAA*A*A |
| SEQ ID: 865 | ATGGACGACGACGACAAGgaaacaggaaccccacAAAAAAAAAAAAAAAAAAAAAAA*A*A |
| SEQ ID: 866 | ATGGACGACGACGACAAGgtatgggaccaacaacAAAAAAAAAAAAAAAAAAAAAAA*A*A |
| SEQ ID: 867 | ATGGACGACGACGACAAGagccgtgagttctccaAAAAAAAAAAAAAAAAAAAAAAA*A*A |
| SEQ ID: 868 | ATGGACGACGACGACAAGagcacggtagtgatgaAAAAAAAAAAAAAAAAAAAAAAA*A*A |
| SEQ ID: 869 | ATGGACGACGACGACAAGctcggcaatgaactgcAAAAAAAAAAAAAAAAAAAAAAA*A*A |
| SEQ ID: 870 | ATGGACGACGACGACAAGttcacggggagctacaAAAAAAAAAAAAAAAAAAAAAAA*A*A |
| SEQ ID: 871 | ATGGACGACGACGACAAGcccggaatattccctaAAAAAAAAAAAAAAAAAAAAAAA*A*A |
| SEQ ID: 872 | ATGGACGACGACGACAAGgcatcgtttccaacggAAAAAAAAAAAAAAAAAAAAAAA*A*A |
| SEQ ID: 873 | ATGGACGACGACGACAAGaaagtaagccaaccgcAAAAAAAAAAAAAAAAAAAAAAA*A*A |
| SEQ ID: 874 | ATGGACGACGACGACAAGagcctagcttaatgcgAAAAAAAAAAAAAAAAAAAAAAA*A*A |
| SEQ ID: 875 | ATGGACGACGACGACAAGgttaccctgcttcgagAAAAAAAAAAAAAAAAAAAAAAA*A*A |
| SEQ ID: 876 | ATGGACGACGACGACAAGgagtgaaagtcaccccAAAAAAAAAAAAAAAAAAAAAAA*A*A |
| SEQ ID: 877 | ATGGACGACGACGACAAGctagtctatttgcgacAAAAAAAAAAAAAAAAAAAAAAA*A*A |
| SEQ ID: 878 | ATGGACGACGACGACAAGgttgggtaaacgcagcAAAAAAAAAAAAAAAAAAAAAAA*A*A |
| SEQ ID: 879 | ATGGACGACGACGACAAGtggaactgtatagctgAAAAAAAAAAAAAAAAAAAAAAA*A*A |
| SEQ ID: 880 | ATGGACGACGACGACAAGctgacagttcacccgtAAAAAAAAAAAAAAAAAAAAAAA*A*A |
| SEQ ID: 881 | ATGGACGACGACGACAAGtcaactggcatgtgtaAAAAAAAAAAAAAAAAAAAAAAA*A*A |
| SEQ ID: 882 | ATGGACGACGACGACAAGcctactggtactacgcAAAAAAAAAAAAAAAAAAAAAAA*A*A |
| SEQ ID: 883 | ATGGACGACGACGACAAGactaggtgctcagttcAAAAAAAAAAAAAAAAAAAAAAA*A*A |
| SEQ ID: 884 | ATGGACGACGACGACAAGaagcgtgttgctgcagAAAAAAAAAAAAAAAAAAAAAAA*A*A |
| SEQ ID: 885 | ATGGACGACGACGACAAGcagctgagatcaggtcAAAAAAAAAAAAAAAAAAAAAAA*A*A |
| SEQ ID: 886 | ATGGACGACGACGACAAGgcactgcttatagaagAAAAAAAAAAAAAAAAAAAAAAA*A*A |
| SEQ ID: 887 | ATGGACGACGACGACAAGtgatgtacgattggagAAAAAAAAAAAAAAAAAAAAAAA*A*A |
| SEQ ID: 888 | ATGGACGACGACGACAAGttcagtggacatcctcAAAAAAAAAAAAAAAAAAAAAAA*A*A |
| SEQ ID: 889 | ATGGACGACGACGACAAGgttttaggtagggaagAAAAAAAAAAAAAAAAAAAAAAA*A*A |
| SEQ ID: 890 | ATGGACGACGACGACAAGtgtgacaagcatgagtAAAAAAAAAAAAAAAAAAAAAAA*A*A |
| SEQ ID: 891 | ATGGACGACGACGACAAGggattcccctaagcagAAAAAAAAAAAAAAAAAAAAAAA*A*A |
| SEQ ID: 892 | ATGGACGACGACGACAAGcagcctatcgaccaagAAAAAAAAAAAAAAAAAAAAAAA*A*A |
| SEQ ID: 893 | ATGGACGACGACGACAAGtatcggtagtccctctAAAAAAAAAAAAAAAAAAAAAAA*A*A |
| SEQ ID: 894 | ATGGACGACGACGACAAGttacgcgttcagacggAAAAAAAAAAAAAAAAAAAAAAA*A*A |
| SEQ ID: 895 | ATGGACGACGACGACAAGatgaggtagctccaccAAAAAAAAAAAAAAAAAAAAAAA*A*A |
| SEQ ID: 896 | ATGGACGACGACGACAAGggggagtgtgtgtataAAAAAAAAAAAAAAAAAAAAAAA*A*A |
| SEQ ID: 897 | ATGGACGACGACGACAAGgttcgggcttttcgacAAAAAAAAAAAAAAAAAAAAAAA*A*A |
| SEQ ID: 898 | ATGGACGACGACGACAAGtgcgcagaaacctcgaAAAAAAAAAAAAAAAAAAAAAAA*A*A |
| SEQ ID: 899 | ATGGACGACGACGACAAGcggtaccgtttcacgaAAAAAAAAAAAAAAAAAAAAAAA*A*A |
| SEQ ID: 900 | ATGGACGACGACGACAAGccgattgatgaacgtcAAAAAAAAAAAAAAAAAAAAAAA*A*A |
| SEQ ID: 901 | ATGGACGACGACGACAAGatcacctgaggaactaAAAAAAAAAAAAAAAAAAAAAAA*A*A |
| SEQ ID: 902 | ATGGACGACGACGACAAGctcgaattagcgcggaAAAAAAAAAAAAAAAAAAAAAAA*A*A |
| SEQ ID: 903 | ATGGACGACGACGACAAGatacagagacgaccatAAAAAAAAAAAAAAAAAAAAAAA*A*A |
| SEQ ID: 904 | ATGGACGACGACGACAAGggtacactgaaatggtAAAAAAAAAAAAAAAAAAAAAAA*A*A |
| SEQ ID: 905 | ATGGACGACGACGACAAGcaggatgaacctatacAAAAAAAAAAAAAAAAAAAAAAA*A*A |
| SEQ ID: 906 | ATGGACGACGACGACAAGcagatggccgataagaAAAAAAAAAAAAAAAAAAAAAAA*A*A |
| SEQ ID: 907 | ATGGACGACGACGACAAGctagtgagggcgcattAAAAAAAAAAAAAAAAAAAAAAA*A*A |
| SEQ ID: 908 | ATGGACGACGACGACAAGtgatacgactagcgccAAAAAAAAAAAAAAAAAAAAAAA*A*A |
| SEQ ID: 909 | ATGGACGACGACGACAAGgatcacctgcaggctaAAAAAAAAAAAAAAAAAAAAAAA*A*A |
| SEQ ID: 910 | ATGGACGACGACGACAAGgcatgttgccagaagaAAAAAAAAAAAAAAAAAAAAAAA*A*A |
| SEQ ID: 911 | ATGGACGACGACGACAAGgagacgtagtactatgAAAAAAAAAAAAAAAAAAAAAAA*A*A |
| SEQ ID: 912 | ATGGACGACGACGACAAGtccagctcaacaacgtAAAAAAAAAAAAAAAAAAAAAAA*A*A |
| SEQ ID: 913 | ATGGACGACGACGACAAGcagtgcctgagatgacAAAAAAAAAAAAAAAAAAAAAAA*A*A |
| SEQ ID: 914 | ATGGACGACGACGACAAGagcacctctaagtcggAAAAAAAAAAAAAAAAAAAAAAA*A*A |
| SEQ ID: 915 | ATGGACGACGACGACAAGttgcgttagagtgtcgAAAAAAAAAAAAAAAAAAAAAAA*A*A |
| SEQ ID: 916 | ATGGACGACGACGACAAGgtcaaatcgtctgcacAAAAAAAAAAAAAAAAAAAAAAA*A*A |
| SEQ ID: 917 | ATGGACGACGACGACAAGgcaacttgtgcctacaAAAAAAAAAAAAAAAAAAAAAAA*A*A |
| SEQ ID: 918 | ATGGACGACGACGACAAGcgagcaaagtgtccttAAAAAAAAAAAAAAAAAAAAAAA*A*A |
| SEQ ID: 919 | ATGGACGACGACGACAAGcatgaaagacacgacgAAAAAAAAAAAAAAAAAAAAAAA*A*A |
| SEQ ID: 920 | ATGGACGACGACGACAAGaggagtatctcacacaAAAAAAAAAAAAAAAAAAAAAAA*A*A |
| SEQ ID: 921 | ATGGACGACGACGACAAGtcgtgcatacctagagAAAAAAAAAAAAAAAAAAAAAAA*A*A |
| SEQ ID: 922 | ATGGACGACGACGACAAGctcattcccagatcggAAAAAAAAAAAAAAAAAAAAAAA*A*A |
| SEQ ID: 923 | ATGGACGACGACGACAAGtacctagcaaggacggAAAAAAAAAAAAAAAAAAAAAAA*A*A |
| SEQ ID: 924 | ATGGACGACGACGACAAGtacagagtccgctgttAAAAAAAAAAAAAAAAAAAAAAA*A*A |
| SEQ ID: 925 | ATGGACGACGACGACAAGctgttggaatttctggAAAAAAAAAAAAAAAAAAAAAAA*A*A |
| SEQ ID: 926 | ATGGACGACGACGACAAGtaggccgaagtaccacAAAAAAAAAAAAAAAAAAAAAAA*A*A |
| SEQ ID: 927 | ATGGACGACGACGACAAGcacgtaacgagtttgcAAAAAAAAAAAAAAAAAAAAAAA*A*A |
| SEQ ID: 928 | ATGGACGACGACGACAAGggtcctaatctatgtgAAAAAAAAAAAAAAAAAAAAAAA*A*A |
| SEQ ID: 929 | ATGGACGACGACGACAAGgagcgtgcagattaccAAAAAAAAAAAAAAAAAAAAAAA*A*A |
| SEQ ID: 930 | ATGGACGACGACGACAAGtcactcgaacggagacAAAAAAAAAAAAAAAAAAAAAAA*A*A |
| SEQ ID: 931 | ATGGACGACGACGACAAGtgggcaacagagtaggAAAAAAAAAAAAAAAAAAAAAAA*A*A |
| SEQ ID: 932 | ATGGACGACGACGACAAGtgatatggagacaccaAAAAAAAAAAAAAAAAAAAAAAA*A*A |
| SEQ ID: 933 | ATGGACGACGACGACAAGcattgtggcaagactgAAAAAAAAAAAAAAAAAAAAAAA*A*A |
| SEQ ID: 934 | ATGGACGACGACGACAAGttatgactaccgcacaAAAAAAAAAAAAAAAAAAAAAAA*A*A |
| SEQ ID: 935 | ATGGACGACGACGACAAGtatgcggaacgttgtgAAAAAAAAAAAAAAAAAAAAAAA*A*A |
| SEQ ID: 936 | ATGGACGACGACGACAAGccattgcgtcttgtccAAAAAAAAAAAAAAAAAAAAAAA*A*A |
| SEQ ID: 937 | ATGGACGACGACGACAAGtggcgctgcgtataatAAAAAAAAAAAAAAAAAAAAAAA*A*A |
| SEQ ID: 938 | ATGGACGACGACGACAAGtgccttacgacacgtaAAAAAAAAAAAAAAAAAAAAAAA*A*A |
| SEQ ID: 939 | ATGGACGACGACGACAAGgtttgggtaggagggaAAAAAAAAAAAAAAAAAAAAAAA*A*A |
| SEQ ID: 940 | ATGGACGACGACGACAAGgttcgttttcggtgccAAAAAAAAAAAAAAAAAAAAAAA*A*A |
| SEQ ID: 941 | ATGGACGACGACGACAAGatattcgccggcaaatAAAAAAAAAAAAAAAAAAAAAAA*A*A |
| SEQ ID: 942 | ATGGACGACGACGACAAGgggaatcatttgctccAAAAAAAAAAAAAAAAAAAAAAA*A*A |
| SEQ ID: 943 | ATGGACGACGACGACAAGccacggaactcgatgtAAAAAAAAAAAAAAAAAAAAAAA*A*A |
| SEQ ID: 944 | ATGGACGACGACGACAAGgtaatctttgctctcgAAAAAAAAAAAAAAAAAAAAAAA*A*A |
| SEQ ID: 945 | ATGGACGACGACGACAAGaagtgcggtatcgaggAAAAAAAAAAAAAAAAAAAAAAA*A*A |
| SEQ ID: 946 | ATGGACGACGACGACAAGgggctgcaagttcacaAAAAAAAAAAAAAAAAAAAAAAA*A*A |
| SEQ ID: 947 | ATGGACGACGACGACAAGaacccaagcagctatcAAAAAAAAAAAAAAAAAAAAAAA*A*A |
| SEQ ID: 948 | ATGGACGACGACGACAAGgatggagaggttgaatAAAAAAAAAAAAAAAAAAAAAAA*A*A |
| SEQ ID: 949 | ATGGACGACGACGACAAGttagaggttgacggtaAAAAAAAAAAAAAAAAAAAAAAA*A*A |
| SEQ ID: 950 | ATGGACGACGACGACAAGgataatctccgacggcAAAAAAAAAAAAAAAAAAAAAAA*A*A |
| SEQ ID: 951 | ATGGACGACGACGACAAGagattagtgctcccgaAAAAAAAAAAAAAAAAAAAAAAA*A*A |
| SEQ ID: 952 | ATGGACGACGACGACAAGactccagttcttgtacAAAAAAAAAAAAAAAAAAAAAAA*A*A |
| SEQ ID: 953 | ATGGACGACGACGACAAGcaccctactcaaagacAAAAAAAAAAAAAAAAAAAAAAA*A*A |
| SEQ ID: 954 | ATGGACGACGACGACAAGtacctcatacgcgttgAAAAAAAAAAAAAAAAAAAAAAA*A*A |
| SEQ ID: 955 | ATGGACGACGACGACAAGcgaaaatcgggtagatAAAAAAAAAAAAAAAAAAAAAAA*A*A |
| SEQ ID: 956 | ATGGACGACGACGACAAGcgatcgctcctaccatAAAAAAAAAAAAAAAAAAAAAAA*A*A |
| SEQ ID: 957 | ATGGACGACGACGACAAGcccactccatactagaAAAAAAAAAAAAAAAAAAAAAAA*A*A |
| SEQ ID: 958 | ATGGACGACGACGACAAGacggctttacgcaagaAAAAAAAAAAAAAAAAAAAAAAA*A*A |
| SEQ ID: 959 | ATGGACGACGACGACAAGtcgcagaaccatctgcAAAAAAAAAAAAAAAAAAAAAAA*A*A |
| SEQ ID: 960 | ATGGACGACGACGACAAGgagttgctagcctgtaAAAAAAAAAAAAAAAAAAAAAAA*A*A |
| SEQ ID: 961 | ATGGACGACGACGACAAGttaactgcttcagccgAAAAAAAAAAAAAAAAAAAAAAA*A*A |
| SEQ ID: 962 | ATGGACGACGACGACAAGtcgcgatgaccgctatAAAAAAAAAAAAAAAAAAAAAAA*A*A |
| SEQ ID: 963 | ATGGACGACGACGACAAGgacgaacgcgttaccaAAAAAAAAAAAAAAAAAAAAAAA*A*A |
| SEQ ID: 964 | ATGGACGACGACGACAAGcggcaaaactactgtcAAAAAAAAAAAAAAAAAAAAAAA*A*A |
| SEQ ID: 965 | ATGGACGACGACGACAAGcccgactctgatgaagAAAAAAAAAAAAAAAAAAAAAAA*A*A |
| SEQ ID: 966 | ATGGACGACGACGACAAGactgcgctacagagtcAAAAAAAAAAAAAAAAAAAAAAA*A*A |
| SEQ ID: 967 | ATGGACGACGACGACAAGacggtgtaccttagggAAAAAAAAAAAAAAAAAAAAAAA*A*A |
| SEQ ID: 968 | ATGGACGACGACGACAAGtcgagtccgcagtatcAAAAAAAAAAAAAAAAAAAAAAA*A*A |
| SEQ ID: 969 | ATGGACGACGACGACAAGgacgctgcctaattggAAAAAAAAAAAAAAAAAAAAAAA*A*A |
| SEQ ID: 970 | ATGGACGACGACGACAAGtggggatggactagtaAAAAAAAAAAAAAAAAAAAAAAA*A*A |
| SEQ ID: 971 | ATGGACGACGACGACAAGgctctaaaggccacagAAAAAAAAAAAAAAAAAAAAAAA*A*A |
| SEQ ID: 972 | ATGGACGACGACGACAAGcaggagtggtgccttaAAAAAAAAAAAAAAAAAAAAAAA*A*A |
| SEQ ID: 973 | ATGGACGACGACGACAAGccgagaagtgttttgaAAAAAAAAAAAAAAAAAAAAAAA*A*A |
| SEQ ID: 974 | ATGGACGACGACGACAAGtgttcaagccacctagAAAAAAAAAAAAAAAAAAAAAAA*A*A |
| SEQ ID: 975 | ATGGACGACGACGACAAGctcccttgagtgtagcAAAAAAAAAAAAAAAAAAAAAAA*A*A |
| SEQ ID: 976 | ATGGACGACGACGACAAGaatgagcactaccgacAAAAAAAAAAAAAAAAAAAAAAA*A*A |
| SEQ ID: 977 | ATGGACGACGACGACAAGacgcaagtcgcaaagcAAAAAAAAAAAAAAAAAAAAAAA*A*A |
| SEQ ID: 978 | ATGGACGACGACGACAAGattgggagagtcagttAAAAAAAAAAAAAAAAAAAAAAA*A*A |
| SEQ ID: 979 | ATGGACGACGACGACAAGgcgacctatataaagcAAAAAAAAAAAAAAAAAAAAAAA*A*A |
| SEQ ID: 980 | ATGGACGACGACGACAAGatccgccacttcagatAAAAAAAAAAAAAAAAAAAAAAA*A*A |
| SEQ ID: 981 | ATGGACGACGACGACAAGtaagcgggttcctattAAAAAAAAAAAAAAAAAAAAAAA*A*A |
| SEQ ID: 982 | ATGGACGACGACGACAAGaccctacgtaccgtcaAAAAAAAAAAAAAAAAAAAAAAA*A*A |
| SEQ ID: 983 | ATGGACGACGACGACAAGtgcgccatcggttttcAAAAAAAAAAAAAAAAAAAAAAA*A*A |
| SEQ ID: 984 | ATGGACGACGACGACAAGgcctaacttctgcctcAAAAAAAAAAAAAAAAAAAAAAA*A*A |
| SEQ ID: 985 | ATGGACGACGACGACAAGgtcctttaatcccctaAAAAAAAAAAAAAAAAAAAAAAA*A*A |
| SEQ ID: 986 | ATGGACGACGACGACAAGgattgtctagacgtagAAAAAAAAAAAAAAAAAAAAAAA*A*A |
| SEQ ID: 987 | ATGGACGACGACGACAAGaacccgcaaaatcctaAAAAAAAAAAAAAAAAAAAAAAA*A*A |
| SEQ ID: 988 | ATGGACGACGACGACAAGtacaacaccaacgctcAAAAAAAAAAAAAAAAAAAAAAA*A*A |
| SEQ ID: 989 | ATGGACGACGACGACAAGtgtgctattgtctccaAAAAAAAAAAAAAAAAAAAAAAA*A*A |
| SEQ ID: 990 | ATGGACGACGACGACAAGagatccacacccggttAAAAAAAAAAAAAAAAAAAAAAA*A*A |
| SEQ ID: 991 | ATGGACGACGACGACAAGgtggtctccaccatcaAAAAAAAAAAAAAAAAAAAAAAA*A*A |
| SEQ ID: 992 | ATGGACGACGACGACAAGgatattccgtcaaaccAAAAAAAAAAAAAAAAAAAAAAA*A*A |
| SEQ ID: 993 | ATGGACGACGACGACAAGacatcgtcgcggattaAAAAAAAAAAAAAAAAAAAAAAA*A*A |
| SEQ ID: 994 | ATGGACGACGACGACAAGaacggtatttggcggcAAAAAAAAAAAAAAAAAAAAAAA*A*A |
| SEQ ID: 995 | ATGGACGACGACGACAAGcgctggattgcaaatgAAAAAAAAAAAAAAAAAAAAAAA*A*A |
| SEQ ID: 996 | ATGGACGACGACGACAAGcaaaggggttacatcgAAAAAAAAAAAAAAAAAAAAAAA*A*A |
| SEQ ID: 997 | ATGGACGACGACGACAAGcgagcagttcaaggagAAAAAAAAAAAAAAAAAAAAAAA*A*A |
| SEQ ID: 998 | ATGGACGACGACGACAAGagtagggtccagcatgAAAAAAAAAAAAAAAAAAAAAAA*A*A |
| SEQ ID: 999 | ATGGACGACGACGACAAGatgcttgcccagtctaAAAAAAAAAAAAAAAAAAAAAAA*A*A |
| SEQ ID: | ATGGACGACGACGACAAGtcgtaaatctaggcgaAAAAAAAAAAAAAAAAAAAAAAA*A*A |
| 1000 | |
| SEQ ID: | ATGGACGACGACGACAAGtggtgacatagagcgcAAAAAAAAAAAAAAAAAAAAAAA*A*A |
| 1001 | |
| SEQ ID: | ATGGACGACGACGACAAGttggtcgacttcgaagAAAAAAAAAAAAAAAAAAAAAAA*A*A |
| 1002 | |
| SEQ ID: | ATGGACGACGACGACAAGccacttaccgtctctcAAAAAAAAAAAAAAAAAAAAAAA*A*A |
| 1003 | |
| SEQ ID: | ATGGACGACGACGACAAGtgtcctaagtcgacgaAAAAAAAAAAAAAAAAAAAAAAA*A*A |
| 1004 | |
| SEQ ID: | ATGGACGACGACGACAAGgcgaacggacgataacAAAAAAAAAAAAAAAAAAAAAAA*A*A |
| 1005 | |
| SEQ ID: | ATGGACGACGACGACAAGacggtgagtaaccatgAAAAAAAAAAAAAAAAAAAAAAA*A*A |
| 1006 | |
| SEQ ID: | ATGGACGACGACGACAAGgaatgtgagacgggctAAAAAAAAAAAAAAAAAAAAAAA*A*A |
| 1007 | |
| SEQ ID: | ATGGACGACGACGACAAGgattggtgtgctcgcaAAAAAAAAAAAAAAAAAAAAAAA*A*A |
| 1008 | |
| SEQ ID: | ATGGACGACGACGACAAGcggacttcttacgttcAAAAAAAAAAAAAAAAAAAAAAA*A*A |
| 1009 | |
| SEQ ID: | ATGGACGACGACGACAAGacatccaaaggctccaAAAAAAAAAAAAAAAAAAAAAAA*A*A |
| 1010 | |
| SEQ ID: | ATGGACGACGACGACAAGttagagtccttacacgAAAAAAAAAAAAAAAAAAAAAAA*A*A |
| 1011 | |
| SEQ ID: | ATGGACGACGACGACAAGacgctcaaggttgtgtAAAAAAAAAAAAAAAAAAAAAAA*A*A |
| 1012 | |
| SEQ ID: | ATGGACGACGACGACAAGcggggcctaataatggAAAAAAAAAAAAAAAAAAAAAAA*A*A |
| 1013 | |
| SEQ ID: | ATGGACGACGACGACAAGccgtaagcctggattgAAAAAAAAAAAAAAAAAAAAAAA*A*A |
| 1014 | |
| SEQ ID: | ATGGACGACGACGACAAGgctacgctatgtgttaAAAAAAAAAAAAAAAAAAAAAAA*A*A |
| 1015 | |
| SEQ ID: | ATGGACGACGACGACAAGaaacaccagtgggtagAAAAAAAAAAAAAAAAAAAAAAA*A*A |
| 1016 | |
| SEQ ID: | ATGGACGACGACGACAAGttgactctaaggcaggAAAAAAAAAAAAAAAAAAAAAAA*A*A |
| 1017 | |
| SEQ ID: | ATGGACGACGACGACAAGcactatttgtcttgggAAAAAAAAAAAAAAAAAAAAAAA*A*A |
| 1018 | |
| SEQ ID: | ATGGACGACGACGACAAGgctacaagttgaccatAAAAAAAAAAAAAAAAAAAAAAA*A*A |
| 1019 | |
| SEQ ID: | ATGGACGACGACGACAAGgcagtagcggatactcAAAAAAAAAAAAAAAAAAAAAAA*A*A |
| 1020 | |
| SEQ ID: | ATGGACGACGACGACAAGaactggtatcgctcacAAAAAAAAAAAAAAAAAAAAAAA*A*A |
| 1021 | |
| SEQ ID: | ATGGACGACGACGACAAGagcttgacgagcctatAAAAAAAAAAAAAAAAAAAAAAA*A*A |
| 1022 | |
| SEQ ID: | ATGGACGACGACGACAAGattgccgatgagtagaAAAAAAAAAAAAAAAAAAAAAAA*A*A |
| 1023 | |
| SEQ ID: | ATGGACGACGACGACAAGaacaggtggttacggtAAAAAAAAAAAAAAAAAAAAAAA*A*A |
| 1024 | |
| SEQ ID: | ATGGACGACGACGACAAGaaactgacgctcgaggAAAAAAAAAAAAAAAAAAAAAAA*A*A |
| 1025 | |
| SEQ ID: | ATGGACGACGACGACAAGcgaaatgtcggctcagAAAAAAAAAAAAAAAAAAAAAAA*A*A |
| 1026 | |
| SEQ ID: | ATGGACGACGACGACAAGtccgatctcagagtttAAAAAAAAAAAAAAAAAAAAAAA*A*A |
| 1027 | |
| SEQ ID: | ATGGACGACGACGACAAGactgcttcgagaagcgAAAAAAAAAAAAAAAAAAAAAAA*A*A |
| 1028 | |
| SEQ ID: | ATGGACGACGACGACAAGgtgatgctgtagggcaAAAAAAAAAAAAAAAAAAAAAAA*A*A |
| 1029 | |
| SEQ ID: | ATGGACGACGACGACAAGagtgggtatgtggtacAAAAAAAAAAAAAAAAAAAAAAA*A*A |
| 1030 | |
| SEQ ID: | ATGGACGACGACGACAAGggagtaagttcaagcaAAAAAAAAAAAAAAAAAAAAAAA*A*A |
| 1031 | |
| SEQ ID: | ATGGACGACGACGACAAGgagcagttttcgccgaAAAAAAAAAAAAAAAAAAAAAAA*A*A |
| 1032 | |
| SEQ ID: | ATGGACGACGACGACAAGcgattacgagtctaggAAAAAAAAAAAAAAAAAAAAAAA*A*A |
| 1033 | |
| SEQ ID: | ATGGACGACGACGACAAGcgcggcacttcttagaAAAAAAAAAAAAAAAAAAAAAAA*A*A |
| 1034 | |
| SEQ ID: | ATGGACGACGACGACAAGggtgcagttcctaagaAAAAAAAAAAAAAAAAAAAAAAA*A*A |
| 1035 | |
| SEQ ID: | ATGGACGACGACGACAAGcgtaggcattagaagaAAAAAAAAAAAAAAAAAAAAAAA*A*A |
| 1036 | |
| SEQ ID: | ATGGACGACGACGACAAGgctatcatcagcgcctAAAAAAAAAAAAAAAAAAAAAAA*A*A |
| 1037 | |
| SEQ ID: | ATGGACGACGACGACAAGgcgggtaggtctaaatAAAAAAAAAAAAAAAAAAAAAAA*A*A |
| 1038 | |
| SEQ ID: | ATGGACGACGACGACAAGcgtccctttgaacattAAAAAAAAAAAAAAAAAAAAAAA*A*A |
| 1039 | |
| SEQ ID: | ATGGACGACGACGACAAGtcagactgcgagacttAAAAAAAAAAAAAAAAAAAAAAA*A*A |
| 1040 | |
| SEQ ID: | ATGGACGACGACGACAAGtgtgttcgttatcggtAAAAAAAAAAAAAAAAAAAAAAA*A*A |
| 1041 | |
| SEQ ID: | ATGGACGACGACGACAAGcctaacagcgtaagcaAAAAAAAAAAAAAAAAAAAAAAA*A*A |
| 1042 | |
| SEQ ID: | ATGGACGACGACGACAAGcctgacatttccgtcaAAAAAAAAAAAAAAAAAAAAAAA*A*A |
| 1043 | |
| SEQ ID: | ATGGACGACGACGACAAGcgaaaccatcgccaatAAAAAAAAAAAAAAAAAAAAAAA*A*A |
| 1044 | |
| SEQ ID: | ATGGACGACGACGACAAGgatcacagaagagtgcAAAAAAAAAAAAAAAAAAAAAAA*A*A |
| 1045 | |
| SEQ ID: | ATGGACGACGACGACAAGacgatacagagcaggtAAAAAAAAAAAAAAAAAAAAAAA*A*A |
| 1046 | |
| SEQ ID: | ATGGACGACGACGACAAGgtcaggaacgagtcttAAAAAAAAAAAAAAAAAAAAAAA*A*A |
| 1047 | |
| SEQ ID: | ATGGACGACGACGACAAGatactgattccctgtcAAAAAAAAAAAAAAAAAAAAAAA*A*A |
| 1048 | |
| SEQ ID: | ATGGACGACGACGACAAGttttcgccatggttgtAAAAAAAAAAAAAAAAAAAAAAA*A*A |
| 1049 | |
| SEQ ID: | ATGGACGACGACGACAAGgttcctacgaacaactAAAAAAAAAAAAAAAAAAAAAAA*A*A |
| 1050 | |
| SEQ ID: | ATGGACGACGACGACAAGtcgataacgctactacAAAAAAAAAAAAAAAAAAAAAAA*A*A |
| 1051 | |
| SEQ ID: | ATGGACGACGACGACAAGtggaacacctgaagttAAAAAAAAAAAAAAAAAAAAAAA*A*A |
| 1052 | |
| SEQ ID: | ATGGACGACGACGACAAGcacgacgtgaaactctAAAAAAAAAAAAAAAAAAAAAAA*A*A |
| 1053 | |
| SEQ ID: | ATGGACGACGACGACAAGatccagtttcaagaggAAAAAAAAAAAAAAAAAAAAAAA*A*A |
| 1054 | |
| SEQ ID: | ATGGACGACGACGACAAGctgcggcgatctttcaAAAAAAAAAAAAAAAAAAAAAAA*A*A |
| 1055 | |
| SEQ ID: | ATGGACGACGACGACAAGcggacttgacttccagAAAAAAAAAAAAAAAAAAAAAAA*A*A |
| 1056 | |
| SEQ ID: | ATGGACGACGACGACAAGgtgtgaatgcataagcAAAAAAAAAAAAAAAAAAAAAAA*A*A |
| 1057 | |
| SEQ ID: | ATGGACGACGACGACAAGtcaccgtgttaggtcaAAAAAAAAAAAAAAAAAAAAAAA*A*A |
| 1058 | |
| SEQ ID: | ATGGACGACGACGACAAGggcatgattgtcgcacAAAAAAAAAAAAAAAAAAAAAAA*A*A |
| 1059 | |
| SEQ ID: | ATGGACGACGACGACAAGgcctagggacacgattAAAAAAAAAAAAAAAAAAAAAAA*A*A |
| 1060 | |
| SEQ ID: | ATGGACGACGACGACAAGacagtccaccatgatcAAAAAAAAAAAAAAAAAAAAAAA*A*A |
| 1061 | |
| SEQ ID: | ATGGACGACGACGACAAGcaaccagtatagaagcAAAAAAAAAAAAAAAAAAAAAAA*A*A |
| 1062 | |
| SEQ ID: | ATGGACGACGACGACAAGtgtaactcacgggttaAAAAAAAAAAAAAAAAAAAAAAA*A*A |
| 1063 | |
| SEQ ID: | ATGGACGACGACGACAAGatagacccttggccctAAAAAAAAAAAAAAAAAAAAAAA*A*A |
| 1064 | |
| SEQ ID: | ATGGACGACGACGACAAGctgtgtatgccctttgAAAAAAAAAAAAAAAAAAAAAAA*A*A |
| 1065 | |
| SEQ ID: | ATGGACGACGACGACAAGatcccaaacttagtgcAAAAAAAAAAAAAAAAAAAAAAA*A*A |
| 1066 | |
| SEQ ID: | ATGGACGACGACGACAAGtcttattacgcccggaAAAAAAAAAAAAAAAAAAAAAAA*A*A |
| 1067 | |
| SEQ ID: | ATGGACGACGACGACAAGacgaatagtgcgccacAAAAAAAAAAAAAAAAAAAAAAA*A*A |
| 1068 | |
| SEQ ID: | ATGGACGACGACGACAAGatgcactgatgatgcgAAAAAAAAAAAAAAAAAAAAAAA*A*A |
| 1069 | |
| SEQ ID: | ATGGACGACGACGACAAGggtaaagtgtcccaagAAAAAAAAAAAAAAAAAAAAAAA*A*A |
| 1070 | |
| SEQ ID: | ATGGACGACGACGACAAGggaagaactagtcccgAAAAAAAAAAAAAAAAAAAAAAA*A*A |
| 1071 | |
| SEQ ID: | ATGGACGACGACGACAAGtagccagatgaaatggAAAAAAAAAAAAAAAAAAAAAAA*A*A |
| 1072 | |
| SEQ ID: | ATGGACGACGACGACAAGacgacacaatgattccAAAAAAAAAAAAAAAAAAAAAAA*A*A |
| 1073 | |
| SEQ ID: | ATGGACGACGACGACAAGccatgtgaaagccaggAAAAAAAAAAAAAAAAAAAAAAA*A*A |
| 1074 | |
| SEQ ID: | ATGGACGACGACGACAAGagggtagaacctcattAAAAAAAAAAAAAAAAAAAAAAA*A*A |
| 1075 | |
| SEQ ID: | ATGGACGACGACGACAAGaacagaaacccgaagaAAAAAAAAAAAAAAAAAAAAAAA*A*A |
| 1076 | |
| SEQ ID: | ATGGACGACGACGACAAGtgggtcggaaatttacAAAAAAAAAAAAAAAAAAAAAAA*A*A |
| 1077 | |
| SEQ ID: | ATGGACGACGACGACAAGccgcagcatacaatccAAAAAAAAAAAAAAAAAAAAAAA*A*A |
| 1078 | |
| SEQ ID: | ATGGACGACGACGACAAGatccagacaacgttgaAAAAAAAAAAAAAAAAAAAAAAA*A*A |
| 1079 | |
| SEQ ID: | ATGGACGACGACGACAAGcaaatggcacgcccttAAAAAAAAAAAAAAAAAAAAAAA*A*A |
| 1080 | |
| SEQ ID: | ATGGACGACGACGACAAGccactcatatacgggtAAAAAAAAAAAAAAAAAAAAAAA*A*A |
| 1081 | |
| SEQ ID: | ATGGACGACGACGACAAGttgaccgtagaatgtgAAAAAAAAAAAAAAAAAAAAAAA*A*A |
| 1082 | |
| SEQ ID: | ATGGACGACGACGACAAGtttcatcggccagtggAAAAAAAAAAAAAAAAAAAAAAA*A*A |
| 1083 | |
| SEQ ID: | ATGGACGACGACGACAAGacgtacccggtagacaAAAAAAAAAAAAAAAAAAAAAAA*A*A |
| 1084 | |
| SEQ ID: | ATGGACGACGACGACAAGgcagggtggaacctatAAAAAAAAAAAAAAAAAAAAAAA*A*A |
| 1085 | |
| SEQ ID: | ATGGACGACGACGACAAGacgtatttattccgccAAAAAAAAAAAAAAAAAAAAAAA*A*A |
| 1086 | |
| SEQ ID: | ATGGACGACGACGACAAGtgtggtcactcggaatAAAAAAAAAAAAAAAAAAAAAAA*A*A |
| 1087 | |
| SEQ ID: | ATGGACGACGACGACAAGctggcatgttgtaggtAAAAAAAAAAAAAAAAAAAAAAA*A*A |
| 1088 | |
| SEQ ID: | ATGGACGACGACGACAAGttaggcaggtgcattgAAAAAAAAAAAAAAAAAAAAAAA*A*A |
| 1089 | |
| SEQ ID: | ATGGACGACGACGACAAGccagaggaaatggggaAAAAAAAAAAAAAAAAAAAAAAA*A*A |
| 1090 | |
| SEQ ID: | ATGGACGACGACGACAAGtgtcaacgcatgaaagAAAAAAAAAAAAAAAAAAAAAAA*A*A |
| 1091 | |
| SEQ ID: | ATGGACGACGACGACAAGcgtttcaatgcagggtAAAAAAAAAAAAAAAAAAAAAAA*A*A |
| 1092 | |
| SEQ ID: | ATGGACGACGACGACAAGgaccccggtaagtttaAAAAAAAAAAAAAAAAAAAAAAA*A*A |
| 1093 | |
| SEQ ID: | ATGGACGACGACGACAAGctcattacggacagtgAAAAAAAAAAAAAAAAAAAAAAA*A*A |
| 1094 | |
| SEQ ID: | ATGGACGACGACGACAAGgggccattagtagtgtAAAAAAAAAAAAAAAAAAAAAAA*A*A |
| 1095 | |
| SEQ ID: | ATGGACGACGACGACAAGttacacctgggaatccAAAAAAAAAAAAAAAAAAAAAAA*A*A |
| 1096 | |
| SEQ ID: | ATGGACGACGACGACAAGctctaccttagtggcgAAAAAAAAAAAAAAAAAAAAAAA*A*A |
| 1097 | |
| SEQ ID: | ATGGACGACGACGACAAGgaattgcggtatcgtcAAAAAAAAAAAAAAAAAAAAAAA*A*A |
| 1098 | |
| SEQ ID: | ATGGACGACGACGACAAGgcctcaacgcaacacaAAAAAAAAAAAAAAAAAAAAAAA*A*A |
| 1099 | |
| SEQ ID: | ATGGACGACGACGACAAGagcgactacagctgagAAAAAAAAAAAAAAAAAAAAAAA*A*A |
| 1100 | |
| SEQ ID: | ATGGACGACGACGACAAGacacacgcaaaacagtAAAAAAAAAAAAAAAAAAAAAAA*A*A |
| 1101 | |
| SEQ ID: | ATGGACGACGACGACAAGgactaagctgcaatccAAAAAAAAAAAAAAAAAAAAAAA*A*A |
| 1102 | |
| SEQ ID: | ATGGACGACGACGACAAGcatacggcgatcttagAAAAAAAAAAAAAAAAAAAAAAA*A*A |
| 1103 | |
| SEQ ID: | ATGGACGACGACGACAAGtatcgtcctatgttcgAAAAAAAAAAAAAAAAAAAAAAA*A*A |
| 1104 | |
| SEQ ID: | ATGGACGACGACGACAAGtaggtccttgggaatgAAAAAAAAAAAAAAAAAAAAAAA*A*A |
| 1105 | |
| SEQ ID: | ATGGACGACGACGACAAGctgagactagcactacAAAAAAAAAAAAAAAAAAAAAAA*A*A |
| 1106 | |
| SEQ ID: | ATGGACGACGACGACAAGgcgtttgagcatccatAAAAAAAAAAAAAAAAAAAAAAA*A*A |
| 1107 | |
| SEQ ID: | ATGGACGACGACGACAAGtaacccaacgcaacctAAAAAAAAAAAAAAAAAAAAAAA*A*A |
| 1108 | |
| SEQ ID: | ATGGACGACGACGACAAGggagttacgcatctggAAAAAAAAAAAAAAAAAAAAAAA*A*A |
| 1109 | |
| SEQ ID: | ATGGACGACGACGACAAGtttgggctcggcctatAAAAAAAAAAAAAAAAAAAAAAA*A*A |
| 1110 | |
| SEQ ID: | ATGGACGACGACGACAAGatgatgagtggaagggAAAAAAAAAAAAAAAAAAAAAAA*A*A |
| 1111 | |
| SEQ ID: | ATGGACGACGACGACAAGgtcagagcactcaaatAAAAAAAAAAAAAAAAAAAAAAA*A*A |
| 1112 | |
| SEQ ID: | ATGGACGACGACGACAAGtgcaagaaacaggcagAAAAAAAAAAAAAAAAAAAAAAA*A*A |
| 1113 | |
| SEQ ID: | ATGGACGACGACGACAAGatggcgttcaggcttcAAAAAAAAAAAAAAAAAAAAAAA*A*A |
| 1114 | |
| SEQ ID: | ATGGACGACGACGACAAGgtttagtcgcgatagcAAAAAAAAAAAAAAAAAAAAAAA*A*A |
| 1115 | |
| SEQ ID: | ATGGACGACGACGACAAGcgcagacccaatgcatAAAAAAAAAAAAAAAAAAAAAAA*A*A |
| 1116 | |
| SEQ ID: | ATGGACGACGACGACAAGtgaaatagtagcgaccAAAAAAAAAAAAAAAAAAAAAAA*A*A |
| 1117 | |
| SEQ ID: | ATGGACGACGACGACAAGcatcgccggctaaatcAAAAAAAAAAAAAAAAAAAAAAA*A*A |
| 1118 | |
| SEQ ID: | ATGGACGACGACGACAAGatgtacgggctctctcAAAAAAAAAAAAAAAAAAAAAAA*A*A |
| 1119 | |
| SEQ ID: | ATGGACGACGACGACAAGccccgttaacatatggAAAAAAAAAAAAAAAAAAAAAAA*A*A |
| 1120 | |
| SEQ ID: | ATGGACGACGACGACAAGgactcgttggcgctatAAAAAAAAAAAAAAAAAAAAAAA*A*A |
| 1121 | |
| SEQ ID: | ATGGACGACGACGACAAGgcccagacctttaggaAAAAAAAAAAAAAAAAAAAAAAA*A*A |
| 1122 | |
| SEQ ID: | ATGGACGACGACGACAAGtcccaacaattaccctAAAAAAAAAAAAAAAAAAAAAAA*A*A |
| 1123 | |
| SEQ ID: | ATGGACGACGACGACAAGcctgtgtgcatctgctAAAAAAAAAAAAAAAAAAAAAAA*A*A |
| 1124 | |
| SEQ ID: | ATGGACGACGACGACAAGggccgttccttggtaaAAAAAAAAAAAAAAAAAAAAAAA*A*A |
| 1125 | |
| SEQ ID: | ATGGACGACGACGACAAGagagtaggttgtgttgAAAAAAAAAAAAAAAAAAAAAAA*A*A |
| 1126 | |
| SEQ ID: | ATGGACGACGACGACAAGactcgataataggacgAAAAAAAAAAAAAAAAAAAAAAA*A*A |
| 1127 | |
| SEQ ID: | ATGGACGACGACGACAAGcccgacgaatggttatAAAAAAAAAAAAAAAAAAAAAAA*A*A |
| 1128 | |
| SEQ ID: | ATGGACGACGACGACAAGcgaccgaatcattcccAAAAAAAAAAAAAAAAAAAAAAA*A*A |
| 1129 | |
| SEQ ID: | ATGGACGACGACGACAAGgcctgtagactttgcaAAAAAAAAAAAAAAAAAAAAAAA*A*A |
| 1130 | |
| SEQ ID: | ATGGACGACGACGACAAGggatccaatacacctaAAAAAAAAAAAAAAAAAAAAAAA*A*A |
| 1131 | |
| SEQ ID: | ATGGACGACGACGACAAGgggagcgaattgtggaAAAAAAAAAAAAAAAAAAAAAAA*A*A |
| 1132 | |
| SEQ ID: | ATGGACGACGACGACAAGcgaccttacggcatgaAAAAAAAAAAAAAAAAAAAAAAA*A*A |
| 1133 | |
| SEQ ID: | ATGGACGACGACGACAAGccgtcacttacgtataAAAAAAAAAAAAAAAAAAAAAAA*A*A |
| 1134 | |
| SEQ ID: | ATGGACGACGACGACAAGcgcagtttcacgtaacAAAAAAAAAAAAAAAAAAAAAAA*A*A |
| 1135 | |
| SEQ ID: | ATGGACGACGACGACAAGggcaagctgaatctacAAAAAAAAAAAAAAAAAAAAAAA*A*A |
| 1136 | |
| SEQ ID: | ATGGACGACGACGACAAGtgcggctacattgccaAAAAAAAAAAAAAAAAAAAAAAA*A*A |
| 1137 | |
| SEQ ID: | ATGGACGACGACGACAAGatcttctcagtcttcgAAAAAAAAAAAAAAAAAAAAAAA*A*A |
| 1138 | |
| SEQ ID: | ATGGACGACGACGACAAGgcaggaagatagtcgtAAAAAAAAAAAAAAAAAAAAAAA*A*A |
| 1139 | |
| SEQ ID: | ATGGACGACGACGACAAGgtgatgtgtctgatacAAAAAAAAAAAAAAAAAAAAAAA*A*A |
| 1140 | |
| SEQ ID: | ATGGACGACGACGACAAGcgtagccaaagtcgtgAAAAAAAAAAAAAAAAAAAAAAA*A*A |
| 1141 | |
| SEQ ID: | ATGGACGACGACGACAAGacttcacggaactacgAAAAAAAAAAAAAAAAAAAAAAA*A*A |
| 1142 | |
| SEQ ID: | ATGGACGACGACGACAAGcgacaaggtatcagttAAAAAAAAAAAAAAAAAAAAAAA*A*A |
| 1143 | |
| SEQ ID: | ATGGACGACGACGACAAGgtatctagggaagtccAAAAAAAAAAAAAAAAAAAAAAA*A*A |
| 1144 | |
| SEQ ID: | ATGGACGACGACGACAAGaagtcagcgaggcgttAAAAAAAAAAAAAAAAAAAAAAA*A*A |
| 1145 | |
| SEQ ID: | ATGGACGACGACGACAAGcgtgtgaccatgatgaAAAAAAAAAAAAAAAAAAAAAAA*A*A |
| 1146 | |
| SEQ ID: | ATGGACGACGACGACAAGacaaagctttcaggctAAAAAAAAAAAAAAAAAAAAAAA*A*A |
| 1147 | |
| SEQ ID: | ATGGACGACGACGACAAGttagtcgtcacatcgcAAAAAAAAAAAAAAAAAAAAAAA*A*A |
| 1148 | |
| SEQ ID: | ATGGACGACGACGACAAGctagaacatgcttcgtAAAAAAAAAAAAAAAAAAAAAAA*A*A |
| 1149 | |
| SEQ ID: | ATGGACGACGACGACAAGagaaacaacgtcaaggAAAAAAAAAAAAAAAAAAAAAAA*A*A |
| 1150 | |
| SEQ ID: | ATGGACGACGACGACAAGtctgtactagctgcacAAAAAAAAAAAAAAAAAAAAAAA*A*A |
| 1151 | |
| SEQ ID: | ATGGACGACGACGACAAGtgcgcattgatggttgAAAAAAAAAAAAAAAAAAAAAAA*A*A |
| 1152 | |
| SEQ ID: | ATGGACGACGACGACAAGtctacccgactttcccAAAAAAAAAAAAAAAAAAAAAAA*A*A |
| 1153 | |
| SEQ ID: | ATGGACGACGACGACAAGtcgcttgtttgcttcaAAAAAAAAAAAAAAAAAAAAAAA*A*A |
| 1154 | |
| SEQ ID: | ATGGACGACGACGACAAGccggtcaagcagtacaAAAAAAAAAAAAAAAAAAAAAAA*A*A |
| 1155 | |
| SEQ ID: | ATGGACGACGACGACAAGttctttgaggcactagAAAAAAAAAAAAAAAAAAAAAAA*A*A |
| 1156 | |
| SEQ ID: | ATGGACGACGACGACAAGaaaagcacagttgcctAAAAAAAAAAAAAAAAAAAAAAA*A*A |
| 1157 | |
| SEQ ID: | ATGGACGACGACGACAAGcttctacctcgaggatAAAAAAAAAAAAAAAAAAAAAAA*A*A |
| 1158 | |
| SEQ ID: | ATGGACGACGACGACAAGggttccaaccttatcaAAAAAAAAAAAAAAAAAAAAAAA*A*A |
| 1159 | |
| SEQ ID: | ATGGACGACGACGACAAGctatgaccgggtgttcAAAAAAAAAAAAAAAAAAAAAAA*A*A |
| 1160 | |
| SEQ ID: | ATGGACGACGACGACAAGgagatcaggagttctaAAAAAAAAAAAAAAAAAAAAAAA*A*A |
| 1161 | |
| SEQ ID: | ATGGACGACGACGACAAGcggagatctgcagactAAAAAAAAAAAAAAAAAAAAAAA*A*A |
| 1162 | |
| SEQ ID: | ATGGACGACGACGACAAGtcttgcgatatgtctcAAAAAAAAAAAAAAAAAAAAAAA*A*A |
| 1163 | |
| SEQ ID: | ATGGACGACGACGACAAGctgtaacaactcggttAAAAAAAAAAAAAAAAAAAAAAA*A*A |
| 1164 | |
| SEQ ID: | ATGGACGACGACGACAAGggttacacgacttgctAAAAAAAAAAAAAAAAAAAAAAA*A*A |
| 1165 | |
| SEQ ID: | ATGGACGACGACGACAAGagagggaacattcgtcAAAAAAAAAAAAAAAAAAAAAAA*A*A |
| 1166 | |
| SEQ ID: | ATGGACGACGACGACAAGgggtattgaacaaacgAAAAAAAAAAAAAAAAAAAAAAA*A*A |
| 1167 | |
| SEQ ID: | ATGGACGACGACGACAAGagtgccagactggcaaAAAAAAAAAAAAAAAAAAAAAAA*A*A |
| 1168 | |
| SEQ ID: | ATGGACGACGACGACAAGggtagatgacgaggagAAAAAAAAAAAAAAAAAAAAAAA*A*A |
| 1169 | |
| SEQ ID: | ATGGACGACGACGACAAGcgtcaattctcagccgAAAAAAAAAAAAAAAAAAAAAAA*A*A |
| 1170 | |
| SEQ ID: | ATGGACGACGACGACAAGacgggagtaagtgtcaAAAAAAAAAAAAAAAAAAAAAAA*A*A |
| 1171 | |
| SEQ ID: | ATGGACGACGACGACAAGaacacttccagtgtcaAAAAAAAAAAAAAAAAAAAAAAA*A*A |
| 1172 | |
| SEQ ID: | ATGGACGACGACGACAAGcatggcggccatttcaAAAAAAAAAAAAAAAAAAAAAAA*A*A |
| 1173 | |
| SEQ ID: | ATGGACGACGACGACAAGgctgatctggattgccAAAAAAAAAAAAAAAAAAAAAAA*A*A |
| 1174 | |
| SEQ ID: | ATGGACGACGACGACAAGcgttaagtgcggtcctAAAAAAAAAAAAAAAAAAAAAAA*A*A |
| 1175 | |
| SEQ ID: | ATGGACGACGACGACAAGgcccatagtgaaacggAAAAAAAAAAAAAAAAAAAAAAA*A*A |
| 1176 | |
| SEQ ID: | ATGGACGACGACGACAAGcagaataggcaagcttAAAAAAAAAAAAAAAAAAAAAAA*A*A |
| 1177 | |
| SEQ ID: | ATGGACGACGACGACAAGtcatcgcacgactgttAAAAAAAAAAAAAAAAAAAAAAA*A*A |
| 1178 | |
| SEQ ID: | ATGGACGACGACGACAAGtccacacttgctagggAAAAAAAAAAAAAAAAAAAAAAA*A*A |
| 1179 | |
| SEQ ID: | ATGGACGACGACGACAAGtaataatagcacgcccAAAAAAAAAAAAAAAAAAAAAAA*A*A |
| 1180 | |
| SEQ ID: | ATGGACGACGACGACAAGgttcaacgccgcttacAAAAAAAAAAAAAAAAAAAAAAA*A*A |
| 1181 | |
| SEQ ID: | ATGGACGACGACGACAAGtcgagctattcccataAAAAAAAAAAAAAAAAAAAAAAA*A*A |
| 1182 | |
| SEQ ID: | ATGGACGACGACGACAAGtcccagtctggacatcAAAAAAAAAAAAAAAAAAAAAAA*A*A |
| 1183 | |
| SEQ ID: | ATGGACGACGACGACAAGccgagatcaaacttcgAAAAAAAAAAAAAAAAAAAAAAA*A*A |
| 1184 | |
| SEQ ID: | ATGGACGACGACGACAAGacgctctaatcgtcgcAAAAAAAAAAAAAAAAAAAAAAA*A*A |
| 1185 | |
| SEQ ID: | ATGGACGACGACGACAAGggtgttaacgagaacgAAAAAAAAAAAAAAAAAAAAAAA*A*A |
| 1186 | |
| SEQ ID: | ATGGACGACGACGACAAGctctatacgggtcagaAAAAAAAAAAAAAAAAAAAAAAA*A*A |
| 1187 | |
| SEQ ID: | ATGGACGACGACGACAAGcatctcccctgtcattAAAAAAAAAAAAAAAAAAAAAAA*A*A |
| 1188 | |
| SEQ ID: | ATGGACGACGACGACAAGgcagatgtgtcggttgAAAAAAAAAAAAAAAAAAAAAAA*A*A |
| 1189 | |
| SEQ ID: | ATGGACGACGACGACAAGacgaacttcccttatgAAAAAAAAAAAAAAAAAAAAAAA*A*A |
| 1190 | |
| SEQ ID: | ATGGACGACGACGACAAGgagtcactccgtcactAAAAAAAAAAAAAAAAAAAAAAA*A*A |
| 1191 | |
| SEQ ID: | ATGGACGACGACGACAAGttcgagacgtgagcgtAAAAAAAAAAAAAAAAAAAAAAA*A*A |
| 1192 | |
| SEQ ID: | ATGGACGACGACGACAAGaatactgtggcacctcAAAAAAAAAAAAAAAAAAAAAAA*A*A |
| 1193 | |
| SEQ ID: | ATGGACGACGACGACAAGcaaagttcagtgtgagAAAAAAAAAAAAAAAAAAAAAAA*A*A |
| 1194 | |
| SEQ ID: | ATGGACGACGACGACAAGatttgccattgccttcAAAAAAAAAAAAAAAAAAAAAAA*A*A |
| 1195 | |
| SEQ ID: | ATGGACGACGACGACAAGacgtaccatatgcgatAAAAAAAAAAAAAAAAAAAAAAA*A*A |
| 1196 | |
| SEQ ID: | ATGGACGACGACGACAAGcccagtcgggaattatAAAAAAAAAAAAAAAAAAAAAAA*A*A |
| 1197 | |
| SEQ ID: | ATGGACGACGACGACAAGgcaatatctatgggccAAAAAAAAAAAAAAAAAAAAAAA*A*A |
| 1198 | |
| SEQ ID: | ATGGACGACGACGACAAGcttgtcctcaagtgagAAAAAAAAAAAAAAAAAAAAAAA*A*A |
| 1199 | |
| SEQ ID: | ATGGACGACGACGACAAGttgctaaacatgggcaAAAAAAAAAAAAAAAAAAAAAAA*A*A |
| 1200 | |
| SEQ ID: | ATGGACGACGACGACAAGtcagagtctaataggcAAAAAAAAAAAAAAAAAAAAAAA*A*A |
| 1201 | |
| SEQ ID: | ATGGACGACGACGACAAGgtggttcccgtttgatAAAAAAAAAAAAAAAAAAAAAAA*A*A |
| 1202 | |
| SEQ ID: | ATGGACGACGACGACAAGgtgtcctgatagggatAAAAAAAAAAAAAAAAAAAAAAA*A*A |
| 1203 | |
| SEQ ID: | ATGGACGACGACGACAAGcttttccagcataccgAAAAAAAAAAAAAAAAAAAAAAA*A*A |
| 1204 | |
| SEQ ID: | ATGGACGACGACGACAAGagtcacggatttctagAAAAAAAAAAAAAAAAAAAAAAA*A*A |
| 1205 | |
| SEQ ID: | ATGGACGACGACGACAAGatgggtcacaaccagtAAAAAAAAAAAAAAAAAAAAAAA*A*A |
| 1206 | |
| SEQ ID: | ATGGACGACGACGACAAGgcacaggacagtaactAAAAAAAAAAAAAAAAAAAAAAA*A*A |
| 1207 | |
| SEQ ID: | ATGGACGACGACGACAAGcatctacaacggaacaAAAAAAAAAAAAAAAAAAAAAAA*A*A |
| 1208 | |
| SEQ ID: | ATGGACGACGACGACAAGataagaccgtaaaggcAAAAAAAAAAAAAAAAAAAAAAA*A*A |
| 1209 | |
| SEQ ID: | ATGGACGACGACGACAAGgctcgcttcgctagttAAAAAAAAAAAAAAAAAAAAAAA*A*A |
| 1210 | |
| SEQ ID: | ATGGACGACGACGACAAGgaaagcctataccactAAAAAAAAAAAAAAAAAAAAAAA*A*A |
| 1211 | |
| SEQ ID: | ATGGACGACGACGACAAGggtaaagacggtgtccAAAAAAAAAAAAAAAAAAAAAAA*A*A |
| 1212 | |
| SEQ ID: | ATGGACGACGACGACAAGttgttcggcctgaggtAAAAAAAAAAAAAAAAAAAAAAA*A*A |
| 1213 | |
| SEQ ID: | ATGGACGACGACGACAAGgtcggctagagaacacAAAAAAAAAAAAAAAAAAAAAAA*A*A |
| 1214 | |
| SEQ ID: | ATGGACGACGACGACAAGagagtccgtgcgatatAAAAAAAAAAAAAAAAAAAAAAA*A*A |
| 1215 | |
| SEQ ID: | ATGGACGACGACGACAAGatatcgcgcagtaccaAAAAAAAAAAAAAAAAAAAAAAA*A*A |
| 1216 | |
| SEQ ID: | ATGGACGACGACGACAAGcaaagctacgggctttAAAAAAAAAAAAAAAAAAAAAAA*A*A |
| 1217 | |
| SEQ ID: | ATGGACGACGACGACAAGaccgcaaaccacatttAAAAAAAAAAAAAAAAAAAAAAA*A*A |
| 1218 | |
| SEQ ID: | ATGGACGACGACGACAAGcggttaagctgattgtAAAAAAAAAAAAAAAAAAAAAAA*A*A |
| 1219 | |
| SEQ ID: | ATGGACGACGACGACAAGtttgtctcacgtccagAAAAAAAAAAAAAAAAAAAAAAA*A*A |
| 1220 | |
| SEQ ID: | ATGGACGACGACGACAAGcttccgcgagcaaaagAAAAAAAAAAAAAAAAAAAAAAA*A*A |
| 1221 | |
| SEQ ID: | ATGGACGACGACGACAAGcaagtcggatctactaAAAAAAAAAAAAAAAAAAAAAAA*A*A |
| 1222 | |
| SEQ ID: | ATGGACGACGACGACAAGaatactcgcgacggctAAAAAAAAAAAAAAAAAAAAAAA*A*A |
| 1223 | |
| SEQ ID: | ATGGACGACGACGACAAGcgcctatcgccgttttAAAAAAAAAAAAAAAAAAAAAAA*A*A |
| 1224 | |
| SEQ ID: | ATGGACGACGACGACAAGgtttactactacacgcAAAAAAAAAAAAAAAAAAAAAAA*A*A |
| 1225 | |
| SEQ ID: | ATGGACGACGACGACAAGgttaaggttacgtcacAAAAAAAAAAAAAAAAAAAAAAA*A*A |
| 1226 | |
| SEQ ID: | ATGGACGACGACGACAAGagctgttcacacgaccAAAAAAAAAAAAAAAAAAAAAAA*A*A |
| 1227 | |
| SEQ ID: | ATGGACGACGACGACAAGcaatactctctggcatAAAAAAAAAAAAAAAAAAAAAAA*A*A |
| 1228 | |
| SEQ ID: | ATGGACGACGACGACAAGttccagtgcatgcgttAAAAAAAAAAAAAAAAAAAAAAA*A*A |
| 1229 | |
| SEQ ID: | ATGGACGACGACGACAAGtgccttttccccgcatAAAAAAAAAAAAAAAAAAAAAAA*A*A |
| 1230 | |
| SEQ ID: | ATGGACGACGACGACAAGcctaacccaaggaagcAAAAAAAAAAAAAAAAAAAAAAA*A*A |
| 1231 | |
| SEQ ID: | ATGGACGACGACGACAAGtagtcttacatctccgAAAAAAAAAAAAAAAAAAAAAAA*A*A |
| 1232 | |
| SEQ ID: | ATGGACGACGACGACAAGctagggtaggctatagAAAAAAAAAAAAAAAAAAAAAAA*A*A |
| 1233 | |
| SEQ ID: | ATGGACGACGACGACAAGtcttgtggaggcttttAAAAAAAAAAAAAAAAAAAAAAA*A*A |
| 1234 | |
| SEQ ID: | ATGGACGACGACGACAAGggaacgagaattacgtAAAAAAAAAAAAAAAAAAAAAAA*A*A |
| 1235 | |
| SEQ ID: | ATGGACGACGACGACAAGggtaagaaatgcttggAAAAAAAAAAAAAAAAAAAAAAA*A*A |
| 1236 | |
| SEQ ID: | ATGGACGACGACGACAAGagtcttcaccaactcaAAAAAAAAAAAAAAAAAAAAAAA*A*A |
| 1237 | |
| SEQ ID: | ATGGACGACGACGACAAGtcaacaaagccttgctAAAAAAAAAAAAAAAAAAAAAAA*A*A |
| 1238 | |
| SEQ ID: | ATGGACGACGACGACAAGggttgctagctctaagAAAAAAAAAAAAAAAAAAAAAAA*A*A |
| 1239 | |
| SEQ ID: | ATGGACGACGACGACAAGcttaccttgttcacctAAAAAAAAAAAAAAAAAAAAAAA*A*A |
| 1240 | |
| SEQ ID: | ATGGACGACGACGACAAGaacatgtagaggggtgAAAAAAAAAAAAAAAAAAAAAAA*A*A |
| 1241 | |
| SEQ ID: | ATGGACGACGACGACAAGttgggttccttcacttAAAAAAAAAAAAAAAAAAAAAAA*A*A |
| 1242 | |
| SEQ ID: | ATGGACGACGACGACAAGgcaccatgctacagtgAAAAAAAAAAAAAAAAAAAAAAA*A*A |
| 1243 | |
| SEQ ID: | ATGGACGACGACGACAAGatgcatgagaaagggaAAAAAAAAAAAAAAAAAAAAAAA*A*A |
| 1244 | |
| SEQ ID: | ATGGACGACGACGACAAGccactagtgagatagaAAAAAAAAAAAAAAAAAAAAAAA*A*A |
| 1245 | |
| SEQ ID: | ATGGACGACGACGACAAGcgacacaccaatattgAAAAAAAAAAAAAAAAAAAAAAA*A*A |
| 1246 | |
| SEQ ID: | ATGGACGACGACGACAAGcagatagtcttgtcacAAAAAAAAAAAAAAAAAAAAAAA*A*A |
| 1247 | |
| SEQ ID: | ATGGACGACGACGACAAGttgtcgagggatacttAAAAAAAAAAAAAAAAAAAAAAA*A*A |
| 1248 | |
| SEQ ID: | ATGGACGACGACGACAAGcgttgagcacctttgcAAAAAAAAAAAAAAAAAAAAAAA*A*A |
| 1249 | |
| SEQ ID: | ATGGACGACGACGACAAGaacagagaagaatcgcAAAAAAAAAAAAAAAAAAAAAAA*A*A |
| 1250 | |
| SEQ ID: | ATGGACGACGACGACAAGgcgtgcttgtactccaAAAAAAAAAAAAAAAAAAAAAAA*A*A |
| 1251 | |
| SEQ ID: | ATGGACGACGACGACAAGttcacgcctcattgatAAAAAAAAAAAAAAAAAAAAAAA*A*A |
| 1252 | |
| SEQ ID: | ATGGACGACGACGACAAGccggcatccgttatacAAAAAAAAAAAAAAAAAAAAAAA*A*A |
| 1253 | |
| SEQ ID: | ATGGACGACGACGACAAGtgagcgttaaccagatAAAAAAAAAAAAAAAAAAAAAAA*A*A |
| 1254 | |
| SEQ ID: | ATGGACGACGACGACAAGtgccgattagcctacgAAAAAAAAAAAAAAAAAAAAAAA*A*A |
| 1255 | |
| SEQ ID: | ATGGACGACGACGACAAGtgttcgtgtggcgcatAAAAAAAAAAAAAAAAAAAAAAA*A*A |
| 1256 | |
| SEQ ID: | ATGGACGACGACGACAAGaccggtagcttatcacAAAAAAAAAAAAAAAAAAAAAAA*A*A |
| 1257 | |
| SEQ ID: | ATGGACGACGACGACAAGacgggagctcactgatAAAAAAAAAAAAAAAAAAAAAAA*A*A |
| 1258 | |
| SEQ ID: | ATGGACGACGACGACAAGgtataactcgagagctAAAAAAAAAAAAAAAAAAAAAAA*A*A |
| 1259 | |
| SEQ ID: | ATGGACGACGACGACAAGcccatcggttatccctAAAAAAAAAAAAAAAAAAAAAAA*A*A |
| 1260 | |
| SEQ ID: | ATGGACGACGACGACAAGagacatgccccgctatAAAAAAAAAAAAAAAAAAAAAAA*A*A |
| 1261 | |
| SEQ ID: | ATGGACGACGACGACAAGgtttctaatcgtccgcAAAAAAAAAAAAAAAAAAAAAAA*A*A |
| 1262 | |
| SEQ ID: | ATGGACGACGACGACAAGgaatgaagcttcgacaAAAAAAAAAAAAAAAAAAAAAAA*A*A |
| 1263 | |
| SEQ ID: | ATGGACGACGACGACAAGgcgattgacccattgaAAAAAAAAAAAAAAAAAAAAAAA*A*A |
| 1264 | |
| SEQ ID: | ATGGACGACGACGACAAGgttggtcctctagagaAAAAAAAAAAAAAAAAAAAAAAA*A*A |
| 1265 | |
| SEQ ID: | ATGGACGACGACGACAAGttgttattcgcccctcAAAAAAAAAAAAAAAAAAAAAAA*A*A |
| 1266 | |
| SEQ ID: | ATGGACGACGACGACAAGattggtgtgtagagctAAAAAAAAAAAAAAAAAAAAAAA*A*A |
| 1267 | |
| SEQ ID: | ATGGACGACGACGACAAGtgccggatgtaattgcAAAAAAAAAAAAAAAAAAAAAAA*A*A |
| 1268 | |
| SEQ ID: | ATGGACGACGACGACAAGagaaacgaaacgttcgAAAAAAAAAAAAAAAAAAAAAAA*A*A |
| 1269 | |
| SEQ ID: | ATGGACGACGACGACAAGcccaaggatggtgctaAAAAAAAAAAAAAAAAAAAAAAA*A*A |
| 1270 | |
| SEQ ID: | ATGGACGACGACGACAAGggaatgggcgagttcaAAAAAAAAAAAAAAAAAAAAAAA*A*A |
| 1271 | |
| SEQ ID: | ATGGACGACGACGACAAGccagcttacccgtattAAAAAAAAAAAAAAAAAAAAAAA*A*A |
| 1272 | |
| SEQ ID: | ATGGACGACGACGACAAGtacgctttaccgtcccAAAAAAAAAAAAAAAAAAAAAAA*A*A |
| 1273 | |
| SEQ ID: | ATGGACGACGACGACAAGgcgcttcgattctattAAAAAAAAAAAAAAAAAAAAAAA*A*A |
| 1274 | |
| SEQ ID: | ATGGACGACGACGACAAGgcaagtgtgggaacgtAAAAAAAAAAAAAAAAAAAAAAA*A*A |
| 1275 | |
| SEQ ID: | ATGGACGACGACGACAAGgaagctcaattggccaAAAAAAAAAAAAAAAAAAAAAAA*A*A |
| 1276 | |
| SEQ ID: | ATGGACGACGACGACAAGttttccaccctgcatcAAAAAAAAAAAAAAAAAAAAAAA*A*A |
| 1277 | |
| SEQ ID: | ATGGACGACGACGACAAGgtcttcgggtgagtttAAAAAAAAAAAAAAAAAAAAAAA*A*A |
| 1278 | |
| SEQ ID: | ATGGACGACGACGACAAGagaatgctgctggtttAAAAAAAAAAAAAAAAAAAAAAA*A*A |
| 1279 | |
| SEQ ID: | ATGGACGACGACGACAAGtgcatcacgttagacgAAAAAAAAAAAAAAAAAAAAAAA*A*A |
| 1280 | |
| SEQ ID: | ATGGACGACGACGACAAGtcgttgccatgaactcAAAAAAAAAAAAAAAAAAAAAAA*A*A |
| 1281 | |
| SEQ ID: | ATGGACGACGACGACAAGtgacgcttgccatctaAAAAAAAAAAAAAAAAAAAAAAA*A*A |
| 1282 | |
| SEQ ID: | ATGGACGACGACGACAAGggcctgtaaggattacAAAAAAAAAAAAAAAAAAAAAAA*A*A |
| 1283 | |
| SEQ ID: | ATGGACGACGACGACAAGgccgattcgattcactAAAAAAAAAAAAAAAAAAAAAAA*A*A |
| 1284 | |
| SEQ ID: | ATGGACGACGACGACAAGggagaaccagaacgacAAAAAAAAAAAAAAAAAAAAAAA*A*A |
| 1285 | |
| SEQ ID: | ATGGACGACGACGACAAGaacgccttttacgtgtAAAAAAAAAAAAAAAAAAAAAAA*A*A |
| 1286 | |
| SEQ ID: | ATGGACGACGACGACAAGaagtcccctctactgcAAAAAAAAAAAAAAAAAAAAAAA*A*A |
| 1287 | |
| SEQ ID: | ATGGACGACGACGACAAGacattcaggtccctccAAAAAAAAAAAAAAAAAAAAAAA*A*A |
| 1288 | |
| SEQ ID: | ATGGACGACGACGACAAGtaggggatggttctggAAAAAAAAAAAAAAAAAAAAAAA*A*A |
| 1289 | |
| SEQ ID: | ATGGACGACGACGACAAGcaagtggatggagaggAAAAAAAAAAAAAAAAAAAAAAA*A*A |
| 1290 | |
| SEQ ID: | ATGGACGACGACGACAAGgctctctacaaaggggAAAAAAAAAAAAAAAAAAAAAAA*A*A |
| 1291 | |
| SEQ ID: | ATGGACGACGACGACAAGgtacaatagacgagtcAAAAAAAAAAAAAAAAAAAAAAA*A*A |
| 1292 | |
| SEQ ID: | ATGGACGACGACGACAAGctaaagtcatcctgccAAAAAAAAAAAAAAAAAAAAAAA*A*A |
| 1293 | |
| SEQ ID: | ATGGACGACGACGACAAGcctattgtactcctcgAAAAAAAAAAAAAAAAAAAAAAA*A*A |
| 1294 | |
| SEQ ID: | ATGGACGACGACGACAAGtatgacgctgtaggcgAAAAAAAAAAAAAAAAAAAAAAA*A*A |
| 1295 | |
| SEQ ID: | ATGGACGACGACGACAAGgctaggtctgactgtcAAAAAAAAAAAAAAAAAAAAAAA*A*A |
| 1296 | |
| SEQ ID: | ATGGACGACGACGACAAGtccagagaatgtgagtAAAAAAAAAAAAAAAAAAAAAAA*A*A |
| 1297 | |
| SEQ ID: | ATGGACGACGACGACAAGtgcttcagtcacagtaAAAAAAAAAAAAAAAAAAAAAAA*A*A |
| 1298 | |
| SEQ ID: | ATGGACGACGACGACAAGttggtgactccgacctAAAAAAAAAAAAAAAAAAAAAAA*A*A |
| 1299 | |
| SEQ ID: | ATGGACGACGACGACAAGgcttcccattcatactAAAAAAAAAAAAAAAAAAAAAAA*A*A |
| 1300 | |
| SEQ ID: | ATGGACGACGACGACAAGtatgtcaactcgcgggAAAAAAAAAAAAAAAAAAAAAAA*A*A |
| 1301 | |
| SEQ ID: | ATGGACGACGACGACAAGaccaacggcttcttgaAAAAAAAAAAAAAAAAAAAAAAA*A*A |
| 1302 | |
| SEQ ID: | ATGGACGACGACGACAAGgtccacccaccatattAAAAAAAAAAAAAAAAAAAAAAA*A*A |
| 1303 | |
| SEQ ID: | ATGGACGACGACGACAAGaaagatcccggctataAAAAAAAAAAAAAAAAAAAAAAA*A*A |
| 1304 | |
| SEQ ID: | ATGGACGACGACGACAAGgggacatcgtttaacaAAAAAAAAAAAAAAAAAAAAAAA*A*A |
| 1305 | |
| SEQ ID: | ATGGACGACGACGACAAGctcgtgcatccacgtaAAAAAAAAAAAAAAAAAAAAAAA*A*A |
| 1306 | |
| SEQ ID: | ATGGACGACGACGACAAGaccggactctggtactAAAAAAAAAAAAAAAAAAAAAAA*A*A |
| 1307 | |
| SEQ ID: | ATGGACGACGACGACAAGctgtagtgcgcagtatAAAAAAAAAAAAAAAAAAAAAAA*A*A |
| 1308 | |
| SEQ ID: | ATGGACGACGACGACAAGacacttcggtgacctgAAAAAAAAAAAAAAAAAAAAAAA*A*A |
| 1309 | |
| SEQ ID: | ATGGACGACGACGACAAGtactgcttccgactgaAAAAAAAAAAAAAAAAAAAAAAA*A*A |
| 1310 | |
| SEQ ID: | ATGGACGACGACGACAAGgtttcagcccaaacttAAAAAAAAAAAAAAAAAAAAAAA*A*A |
| 1311 | |
| SEQ ID: | ATGGACGACGACGACAAGcgtactgacctcgagtAAAAAAAAAAAAAAAAAAAAAAA*A*A |
| 1312 | |
| SEQ ID: | ATGGACGACGACGACAAGgcgtcaaacttttgagAAAAAAAAAAAAAAAAAAAAAAA*A*A |
| 1313 | |
| SEQ ID: | ATGGACGACGACGACAAGatccctttggatccctAAAAAAAAAAAAAAAAAAAAAAA*A*A |
| 1314 | |
| SEQ ID: | ATGGACGACGACGACAAGcttcgttgttcatcgtAAAAAAAAAAAAAAAAAAAAAAA*A*A |
| 1315 | |
| SEQ ID: | ATGGACGACGACGACAAGcgtctaggataccataAAAAAAAAAAAAAAAAAAAAAAA*A*A |
| 1316 | |
| SEQ ID: | ATGGACGACGACGACAAGctaagccaaatctcgcAAAAAAAAAAAAAAAAAAAAAAA*A*A |
| 1317 | |
| SEQ ID: | ATGGACGACGACGACAAGggacgtagagcactagAAAAAAAAAAAAAAAAAAAAAAA*A*A |
| 1318 | |
| SEQ ID: | ATGGACGACGACGACAAGacccctgatagatcttAAAAAAAAAAAAAAAAAAAAAAA*A*A |
| 1319 | |
| SEQ ID: | ATGGACGACGACGACAAGagcactgcggtttgttAAAAAAAAAAAAAAAAAAAAAAA*A*A |
| 1320 | |
| SEQ ID: | ATGGACGACGACGACAAGcgctctatgtaggaatAAAAAAAAAAAAAAAAAAAAAAA*A*A |
| 1321 | |
| SEQ ID: | ATGGACGACGACGACAAGctttgataccatgggaAAAAAAAAAAAAAAAAAAAAAAA*A*A |
| 1322 | |
| SEQ ID: | ATGGACGACGACGACAAGccaccaccatcttctgAAAAAAAAAAAAAAAAAAAAAAA*A*A |
| 1323 | |
| SEQ ID: | ATGGACGACGACGACAAGcagtcgtattgggaccAAAAAAAAAAAAAAAAAAAAAAA*A*A |
| 1324 | |
| SEQ ID: | ATGGACGACGACGACAAGggtgtacatctgttgtAAAAAAAAAAAAAAAAAAAAAAA*A*A |
| 1325 | |
| SEQ ID: | ATGGACGACGACGACAAGcttgtggagagtcgatAAAAAAAAAAAAAAAAAAAAAAA*A*A |
| 1326 | |
| SEQ ID: | ATGGACGACGACGACAAGactttaagcccgcgttAAAAAAAAAAAAAAAAAAAAAAA*A*A |
| 1327 | |
| SEQ ID: | ATGGACGACGACGACAAGgaaaacggtcttccgaAAAAAAAAAAAAAAAAAAAAAAA*A*A |
| 1328 | |
| SEQ ID: | ATGGACGACGACGACAAGcctcactcgtgtttccAAAAAAAAAAAAAAAAAAAAAAA*A*A |
| 1329 | |
| SEQ ID: | ATGGACGACGACGACAAGgttacatccggccagtAAAAAAAAAAAAAAAAAAAAAAA*A*A |
| 1330 | |
| SEQ ID: | ATGGACGACGACGACAAGtccgagataatctaggAAAAAAAAAAAAAAAAAAAAAAA*A*A |
| 1331 | |
| SEQ ID: | ATGGACGACGACGACAAGgcactatcacctcagaAAAAAAAAAAAAAAAAAAAAAAA*A*A |
| 1332 | |
| SEQ ID: | ATGGACGACGACGACAAGtcaggaggtcgtacctAAAAAAAAAAAAAAAAAAAAAAA*A*A |
| 1333 | |
| SEQ ID: | ATGGACGACGACGACAAGaattgtgctcatcgggAAAAAAAAAAAAAAAAAAAAAAA*A*A |
| 1334 | |
| SEQ ID: | ATGGACGACGACGACAAGcggcccgattctaatcAAAAAAAAAAAAAAAAAAAAAAA*A*A |
| 1335 | |
| SEQ ID: | ATGGACGACGACGACAAGtgtatggcagcaagacAAAAAAAAAAAAAAAAAAAAAAA*A*A |
| 1336 | |
| SEQ ID: | ATGGACGACGACGACAAGcaaagaccgacgaattAAAAAAAAAAAAAAAAAAAAAAA*A*A |
| 1337 | |
| SEQ ID: | ATGGACGACGACGACAAGgtgcctctgttcatggAAAAAAAAAAAAAAAAAAAAAAA*A*A |
| 1338 | |
| SEQ ID: | ATGGACGACGACGACAAGgaacgaagtggtagtcAAAAAAAAAAAAAAAAAAAAAAA*A*A |
| 1339 | |
| SEQ ID: | ATGGACGACGACGACAAGgtctcgactagatttgAAAAAAAAAAAAAAAAAAAAAAA*A*A |
| 1340 | |
| SEQ ID: | ATGGACGACGACGACAAGcactcccgaatggtgtAAAAAAAAAAAAAAAAAAAAAAA*A*A |
| 1341 | |
| SEQ ID: | ATGGACGACGACGACAAGaagaaagataaccgcgAAAAAAAAAAAAAAAAAAAAAAA*A*A |
| 1342 | |
| SEQ ID: | ATGGACGACGACGACAAGaaccagagggagggatAAAAAAAAAAAAAAAAAAAAAAA*A*A |
| 1343 | |
| SEQ ID: | ATGGACGACGACGACAAGgctgtcgctacgaattAAAAAAAAAAAAAAAAAAAAAAA*A*A |
| 1344 | |
| SEQ ID: | ATGGACGACGACGACAAGtctcccactggtgactAAAAAAAAAAAAAAAAAAAAAAA*A*A |
| 1345 | |
| SEQ ID: | ATGGACGACGACGACAAGcagactaggaggagagAAAAAAAAAAAAAAAAAAAAAAA*A*A |
| 1346 | |
| SEQ ID: | ATGGACGACGACGACAAGgcagacaggacatcagAAAAAAAAAAAAAAAAAAAAAAA*A*A |
| 1347 | |
| SEQ ID: | ATGGACGACGACGACAAGtccatggaagtgtaccAAAAAAAAAAAAAAAAAAAAAAA*A*A |
| 1348 | |
| SEQ ID: | ATGGACGACGACGACAAGgtcattgactgtagtcAAAAAAAAAAAAAAAAAAAAAAA*A*A |
| 1349 | |
| SEQ ID: | ATGGACGACGACGACAAGctcggaccttttctcgAAAAAAAAAAAAAAAAAAAAAAA*A*A |
| 1350 | |
| SEQ ID: | ATGGACGACGACGACAAGtgctgatggtaaaccgAAAAAAAAAAAAAAAAAAAAAAA*A*A |
| 1351 | |
| SEQ ID: | ATGGACGACGACGACAAGggctttcggtggtacaAAAAAAAAAAAAAAAAAAAAAAA*A*A |
| 1352 | |
| SEQ ID: | ATGGACGACGACGACAAGcacatccaaccagcacAAAAAAAAAAAAAAAAAAAAAAA*A*A |
| 1353 | |
| SEQ ID: | ATGGACGACGACGACAAGaccatcccgaaacgagAAAAAAAAAAAAAAAAAAAAAAA*A*A |
| 1354 | |
| SEQ ID: | ATGGACGACGACGACAAGgagctacctcacattaAAAAAAAAAAAAAAAAAAAAAAA*A*A |
| 1355 | |
| SEQ ID: | ATGGACGACGACGACAAGgatagtaccatgcgttAAAAAAAAAAAAAAAAAAAAAAA*A*A |
| 1356 | |
| SEQ ID: | ATGGACGACGACGACAAGgacataggaggtcatgAAAAAAAAAAAAAAAAAAAAAAA*A*A |
| 1357 | |
| SEQ ID: | ATGGACGACGACGACAAGtgtcgtatcactatccAAAAAAAAAAAAAAAAAAAAAAA*A*A |
| 1358 | |
| SEQ ID: | ATGGACGACGACGACAAGctgcaagtgggcgaatAAAAAAAAAAAAAAAAAAAAAAA*A*A |
| 1359 | |
| SEQ ID: | ATGGACGACGACGACAAGagatccgataacgtacAAAAAAAAAAAAAAAAAAAAAAA*A*A |
| 1360 | |
| SEQ ID: | ATGGACGACGACGACAAGattgtaggtgcccaccAAAAAAAAAAAAAAAAAAAAAAA*A*A |
| 1361 | |
| SEQ ID: | ATGGACGACGACGACAAGaaagtaacaacgggagAAAAAAAAAAAAAAAAAAAAAAA*A*A |
| 1362 | |
| SEQ ID: | ATGGACGACGACGACAAGtttccaatttgcgctcAAAAAAAAAAAAAAAAAAAAAAA*A*A |
| 1363 | |
| SEQ ID: | ATGGACGACGACGACAAGttgcagctctctcgagAAAAAAAAAAAAAAAAAAAAAAA*A*A |
| 1364 | |
| SEQ ID: | ATGGACGACGACGACAAGaccatccttgcatttcAAAAAAAAAAAAAAAAAAAAAAA*A*A |
| 1365 | |
| SEQ ID: | ATGGACGACGACGACAAGtcctcggtttgtccagAAAAAAAAAAAAAAAAAAAAAAA*A*A |
| 1366 | |
| SEQ ID: | ATGGACGACGACGACAAGtactcatccgtgaactAAAAAAAAAAAAAAAAAAAAAAA*A*A |
| 1367 | |
| SEQ ID: | ATGGACGACGACGACAAGtgttacctagtccctgAAAAAAAAAAAAAAAAAAAAAAA*A*A |
| 1368 | |
| SEQ ID: | ATGGACGACGACGACAAGacctataacgtgggcgAAAAAAAAAAAAAAAAAAAAAAA*A*A |
| 1369 | |
| SEQ ID: | ATGGACGACGACGACAAGcaaggttgctgtgtgcAAAAAAAAAAAAAAAAAAAAAAA*A*A |
| 1370 | |
| SEQ ID: | ATGGACGACGACGACAAGacgcagttgcacacttAAAAAAAAAAAAAAAAAAAAAAA*A*A |
| 1371 | |
| SEQ ID: | ATGGACGACGACGACAAGaagggtcaggtgaggaAAAAAAAAAAAAAAAAAAAAAAA*A*A |
| 1372 | |
| SEQ ID: | ATGGACGACGACGACAAGtgttgaggctgcaggaAAAAAAAAAAAAAAAAAAAAAAA*A*A |
| 1373 | |
| SEQ ID: | ATGGACGACGACGACAAGgtccgagtgtattctgAAAAAAAAAAAAAAAAAAAAAAA*A*A |
| 1374 | |
| SEQ ID: | ATGGACGACGACGACAAGtcaagaacctagcgagAAAAAAAAAAAAAAAAAAAAAAA*A*A |
| 1375 | |
| SEQ ID: | ATGGACGACGACGACAAGtcttatatgaggcgtgAAAAAAAAAAAAAAAAAAAAAAA*A*A |
| 1376 | |
| SEQ ID: | ATGGACGACGACGACAAGttatgtcgcgttccgtAAAAAAAAAAAAAAAAAAAAAAA*A*A |
| 1377 | |
| SEQ ID: | ATGGACGACGACGACAAGcattgctcagccacacAAAAAAAAAAAAAAAAAAAAAAA*A*A |
| 1378 | |
| SEQ ID: | ATGGACGACGACGACAAGtttatgcacacttgccAAAAAAAAAAAAAAAAAAAAAAA*A*A |
| 1379 | |
| SEQ ID: | ATGGACGACGACGACAAGagttatcgggcacgatAAAAAAAAAAAAAAAAAAAAAAA*A*A |
| 1380 | |
| SEQ ID: | ATGGACGACGACGACAAGttggcatcccgattctAAAAAAAAAAAAAAAAAAAAAAA*A*A |
| 1381 | |
| SEQ ID: | ATGGACGACGACGACAAGaatgtacgaagtccctAAAAAAAAAAAAAAAAAAAAAAA*A*A |
| 1382 | |
| SEQ ID: | ATGGACGACGACGACAAGgatgaatggccttcttAAAAAAAAAAAAAAAAAAAAAAA*A*A |
| 1383 | |
| SEQ ID: | ATGGACGACGACGACAAGaaacgtcaacctcgccAAAAAAAAAAAAAAAAAAAAAAA*A*A |
| 1384 | |
| SEQ ID: | ATGGACGACGACGACAAGcacgttcgccagaaatAAAAAAAAAAAAAAAAAAAAAAA*A*A |
| 1385 | |
| SEQ ID: | ATGGACGACGACGACAAGcagatctaaatgcacgAAAAAAAAAAAAAAAAAAAAAAA*A*A |
| 1386 | |
| SEQ ID: | ATGGACGACGACGACAAGattctcgcaactgtctAAAAAAAAAAAAAAAAAAAAAAA*A*A |
| 1387 | |
| SEQ ID: | ATGGACGACGACGACAAGagcatggttcccaactAAAAAAAAAAAAAAAAAAAAAAA*A*A |
| 1388 | |
| SEQ ID: | ATGGACGACGACGACAAGagggaatgcttgatctAAAAAAAAAAAAAAAAAAAAAAA*A*A |
| 1389 | |
| SEQ ID: | ATGGACGACGACGACAAGccccacagtattcagcAAAAAAAAAAAAAAAAAAAAAAA*A*A |
| 1390 | |
| SEQ ID: | ATGGACGACGACGACAAGagcgtactggacaagcAAAAAAAAAAAAAAAAAAAAAAA*A*A |
| 1391 | |
| SEQ ID: | ATGGACGACGACGACAAGcggttcatcgttgaccAAAAAAAAAAAAAAAAAAAAAAA*A*A |
| 1392 | |
| SEQ ID: | ATGGACGACGACGACAAGgggtgtactaggtaatAAAAAAAAAAAAAAAAAAAAAAA*A*A |
| 1393 | |
| SEQ ID: | ATGGACGACGACGACAAGccatctggattagactAAAAAAAAAAAAAAAAAAAAAAA*A*A |
| 1394 | |
| SEQ ID: | ATGGACGACGACGACAAGgatgcgaagcgcatacAAAAAAAAAAAAAAAAAAAAAAA*A*A |
| 1395 | |
| SEQ ID: | ATGGACGACGACGACAAGcataccacgcctatgtAAAAAAAAAAAAAAAAAAAAAAA*A*A |
| 1396 | |
| SEQ ID: | ATGGACGACGACGACAAGgaagtggtcttcaggtAAAAAAAAAAAAAAAAAAAAAAA*A*A |
| 1397 | |
| SEQ ID: | ATGGACGACGACGACAAGtcgctgagccgcaaatAAAAAAAAAAAAAAAAAAAAAAA*A*A |
| 1398 | |
| SEQ ID: | ATGGACGACGACGACAAGttatggagcctgttcgAAAAAAAAAAAAAAAAAAAAAAA*A*A |
| 1399 | |
| SEQ ID: | ATGGACGACGACGACAAGgaagcccataggaggtAAAAAAAAAAAAAAAAAAAAAAA*A*A |
| 1400 | |
| SEQ ID: | ATGGACGACGACGACAAGgccgtgacagtggtttAAAAAAAAAAAAAAAAAAAAAAA*A*A |
| 1401 | |
| SEQ ID: | ATGGACGACGACGACAAGaagtcgacctctatcgAAAAAAAAAAAAAAAAAAAAAAA*A*A |
| 1402 | |
| SEQ ID: | ATGGACGACGACGACAAGcattgactttcgagcgAAAAAAAAAAAAAAAAAAAAAAA*A*A |
| 1403 | |
| SEQ ID: | ATGGACGACGACGACAAGattaaacagggagctgAAAAAAAAAAAAAAAAAAAAAAA*A*A |
| 1404 | |
| SEQ ID: | ATGGACGACGACGACAAGacaatccgaggtctgaAAAAAAAAAAAAAAAAAAAAAAA*A*A |
| 1405 | |
| SEQ ID: | ATGGACGACGACGACAAGgaagggcaaggtttctAAAAAAAAAAAAAAAAAAAAAAA*A*A |
| 1406 | |
| SEQ ID: | ATGGACGACGACGACAAGgtggaaaaccgagataAAAAAAAAAAAAAAAAAAAAAAA*A*A |
| 1407 | |
| SEQ ID: | ATGGACGACGACGACAAGaccattactcgtaagcAAAAAAAAAAAAAAAAAAAAAAA*A*A |
| 1408 | |
| SEQ ID: | ATGGACGACGACGACAAGcgtccgatgacctcttAAAAAAAAAAAAAAAAAAAAAAA*A*A |
| 1409 | |
| SEQ ID: | ATGGACGACGACGACAAGtgtggcgcttacaaacAAAAAAAAAAAAAAAAAAAAAAA*A*A |
| 1410 | |
| SEQ ID: | ATGGACGACGACGACAAGattcacatgtgcaggaAAAAAAAAAAAAAAAAAAAAAAA*A*A |
| 1411 | |
| SEQ ID: | ATGGACGACGACGACAAGctaccacacaagctccAAAAAAAAAAAAAAAAAAAAAAA*A*A |
| 1412 | |
| SEQ ID: | ATGGACGACGACGACAAGggatggtaattcgcttAAAAAAAAAAAAAAAAAAAAAAA*A*A |
| 1413 | |
| SEQ ID: | ATGGACGACGACGACAAGttcaaaggtttgacgcAAAAAAAAAAAAAAAAAAAAAAA*A*A |
| 1414 | |
| SEQ ID: | ATGGACGACGACGACAAGgtctgcagcaatctctAAAAAAAAAAAAAAAAAAAAAAA*A*A |
| 1415 | |
| SEQ ID: | ATGGACGACGACGACAAGgacagtcgtaactgggAAAAAAAAAAAAAAAAAAAAAAA*A*A |
| 1416 | |
| SEQ ID: | ATGGACGACGACGACAAGagtgcttgtaaagagcAAAAAAAAAAAAAAAAAAAAAAA*A*A |
| 1417 | |
| SEQ ID: | ATGGACGACGACGACAAGgtaggagctgcctttgAAAAAAAAAAAAAAAAAAAAAAA*A*A |
| 1418 | |
| SEQ ID: | ATGGACGACGACGACAAGccactttcgtagacatAAAAAAAAAAAAAAAAAAAAAAA*A*A |
| 1419 | |
| SEQ ID: | ATGGACGACGACGACAAGtgattagcgtggttacAAAAAAAAAAAAAAAAAAAAAAA*A*A |
| 1420 | |
| SEQ ID: | ATGGACGACGACGACAAGaaaggcagtaagaaccAAAAAAAAAAAAAAAAAAAAAAA*A*A |
| 1421 | |
| SEQ ID: | ATGGACGACGACGACAAGcgtagtttagggcccaAAAAAAAAAAAAAAAAAAAAAAA*A*A |
| 1422 | |
| SEQ ID: | ATGGACGACGACGACAAGgtcataatcccgttccAAAAAAAAAAAAAAAAAAAAAAA*A*A |
| 1423 | |
| SEQ ID: | ATGGACGACGACGACAAGttgatacgttccctggAAAAAAAAAAAAAAAAAAAAAAA*A*A |
| 1424 | |
| SEQ ID: | ATGGACGACGACGACAAGaacgataggatcgcgaAAAAAAAAAAAAAAAAAAAAAAA*A*A |
| 1425 | |
| SEQ ID: | ATGGACGACGACGACAAGagaatttagggcgcctAAAAAAAAAAAAAAAAAAAAAAA*A*A |
| 1426 | |
| SEQ ID: | ATGGACGACGACGACAAGctagcatttagacccaAAAAAAAAAAAAAAAAAAAAAAA*A*A |
| 1427 | |
| SEQ ID: | ATGGACGACGACGACAAGaccgtttgacggtttgAAAAAAAAAAAAAAAAAAAAAAA*A*A |
| 1428 | |
| SEQ ID: | ATGGACGACGACGACAAGgtggtagcatgctagcAAAAAAAAAAAAAAAAAAAAAAA*A*A |
| 1429 | |
| SEQ ID: | ATGGACGACGACGACAAGctgtttcgtaccagtcAAAAAAAAAAAAAAAAAAAAAAA*A*A |
| 1430 | |
| SEQ ID: | ATGGACGACGACGACAAGattacgtccgagagagAAAAAAAAAAAAAAAAAAAAAAA*A*A |
| 1431 | |
| SEQ ID: | ATGGACGACGACGACAAGggacttattcgacactAAAAAAAAAAAAAAAAAAAAAAA*A*A |
| 1432 | |
| SEQ ID: | ATGGACGACGACGACAAGccattgacaggacgagAAAAAAAAAAAAAAAAAAAAAAA*A*A |
| 1433 | |
| SEQ ID: | ATGGACGACGACGACAAGagcgtgaaatcgtgctAAAAAAAAAAAAAAAAAAAAAAA*A*A |
| 1434 | |
| SEQ ID: | ATGGACGACGACGACAAGctggttataaggggttAAAAAAAAAAAAAAAAAAAAAAA*A*A |
| 1435 | |
| SEQ ID: | ATGGACGACGACGACAAGctgcgcatccgtactaAAAAAAAAAAAAAAAAAAAAAAA*A*A |
| 1436 | |
| SEQ ID: | ATGGACGACGACGACAAGatcccacagcctaatgAAAAAAAAAAAAAAAAAAAAAAA*A*A |
| 1437 | |
| SEQ ID: | ATGGACGACGACGACAAGatgcgtaatcaggaacAAAAAAAAAAAAAAAAAAAAAAA*A*A |
| 1438 | |
| SEQ ID: | ATGGACGACGACGACAAGacgccgtgaactgaacAAAAAAAAAAAAAAAAAAAAAAA*A*A |
| 1439 | |
| SEQ ID: | ATGGACGACGACGACAAGatagcccggcaatgcaAAAAAAAAAAAAAAAAAAAAAAA*A*A |
| 1440 | |
| SEQ ID: | ATGGACGACGACGACAAGcacctcaaagtcagccAAAAAAAAAAAAAAAAAAAAAAA*A*A |
| 1441 | |
| SEQ ID: | ATGGACGACGACGACAAGttccaaggacgtggaaAAAAAAAAAAAAAAAAAAAAAAA*A*A |
| 1442 | |
| SEQ ID: | ATGGACGACGACGACAAGagagagatgctaaccgAAAAAAAAAAAAAAAAAAAAAAA*A*A |
| 1443 | |
| SEQ ID: | ATGGACGACGACGACAAGgttccggaactgtcctAAAAAAAAAAAAAAAAAAAAAAA*A*A |
| 1444 | |
| SEQ ID: | ATGGACGACGACGACAAGggatggtcctgaatccAAAAAAAAAAAAAAAAAAAAAAA*A*A |
| 1445 | |
| SEQ ID: | ATGGACGACGACGACAAGattttggcggtgggtcAAAAAAAAAAAAAAAAAAAAAAA*A*A |
| 1446 | |
| SEQ ID: | ATGGACGACGACGACAAGaatcgattgcgtacggAAAAAAAAAAAAAAAAAAAAAAA*A*A |
| 1447 | |
| SEQ ID: | ATGGACGACGACGACAAGtggagccgttattacgAAAAAAAAAAAAAAAAAAAAAAA*A*A |
| 1448 | |
| SEQ ID: | ATGGACGACGACGACAAGaggcattgtgactggtAAAAAAAAAAAAAAAAAAAAAAA*A*A |
| 1449 | |
| SEQ ID: | ATGGACGACGACGACAAGgactgctgtccaaaatAAAAAAAAAAAAAAAAAAAAAAA*A*A |
| 1450 | |
| SEQ ID: | ATGGACGACGACGACAAGccctttgcgtcccattAAAAAAAAAAAAAAAAAAAAAAA*A*A |
| 1451 | |
| SEQ ID: | ATGGACGACGACGACAAGttgcaagcggctaccaAAAAAAAAAAAAAAAAAAAAAAA*A*A |
| 1452 | |
| SEQ ID: | ATGGACGACGACGACAAGttggcgcatttatcggAAAAAAAAAAAAAAAAAAAAAAA*A*A |
| 1453 | |
| SEQ ID: | ATGGACGACGACGACAAGcaacatcttaggtctcAAAAAAAAAAAAAAAAAAAAAAA*A*A |
| 1454 | |
| SEQ ID: | ATGGACGACGACGACAAGgtaatccgtcaggagtAAAAAAAAAAAAAAAAAAAAAAA*A*A |
| 1455 | |
| SEQ ID: | ATGGACGACGACGACAAGcactgtcacgtacacaAAAAAAAAAAAAAAAAAAAAAAA*A*A |
| 1456 | |
| SEQ ID: | ATGGACGACGACGACAAGggtgaggggatagtaaAAAAAAAAAAAAAAAAAAAAAAA*A*A |
| 1457 | |
| SEQ ID: | ATGGACGACGACGACAAGatgggcacatattctcAAAAAAAAAAAAAAAAAAAAAAA*A*A |
| 1458 | |
| SEQ ID: | ATGGACGACGACGACAAGaaaacgcctatcactcAAAAAAAAAAAAAAAAAAAAAAA*A*A |
| 1459 | |
| SEQ ID: | ATGGACGACGACGACAAGctctctttgatccgtaAAAAAAAAAAAAAAAAAAAAAAA*A*A |
| 1460 | |
| SEQ ID: | ATGGACGACGACGACAAGcttacgaggctaccgaAAAAAAAAAAAAAAAAAAAAAAA*A*A |
| 1461 | |
| SEQ ID: | ATGGACGACGACGACAAGtgtctagctgaggcaaAAAAAAAAAAAAAAAAAAAAAAA*A*A |
| 1462 | |
| SEQ ID: | ATGGACGACGACGACAAGgtaggacagatccgcaAAAAAAAAAAAAAAAAAAAAAAA*A*A |
| 1463 | |
| SEQ ID: | ATGGACGACGACGACAAGgtacccatgtcttaacAAAAAAAAAAAAAAAAAAAAAAA*A*A |
| 1464 | |
| SEQ ID: | ATGGACGACGACGACAAGagacctctcggtgaatAAAAAAAAAAAAAAAAAAAAAAA*A*A |
| 1465 | |
| SEQ ID: | ATGGACGACGACGACAAGgggtcgattcacttgaAAAAAAAAAAAAAAAAAAAAAAA*A*A |
| 1466 | |
| SEQ ID: | ATGGACGACGACGACAAGtcgatacgccaaggtgAAAAAAAAAAAAAAAAAAAAAAA*A*A |
| 1467 | |
| SEQ ID: | ATGGACGACGACGACAAGtgtttgtagccgcctgAAAAAAAAAAAAAAAAAAAAAAA*A*A |
| 1468 | |
| SEQ ID: | ATGGACGACGACGACAAGaattctgcctcctcaaAAAAAAAAAAAAAAAAAAAAAAA*A*A |
| 1469 | |
| SEQ ID: | ATGGACGACGACGACAAGctccgaaaagttgcagAAAAAAAAAAAAAAAAAAAAAAA*A*A |
| 1470 | |
| SEQ ID: | ATGGACGACGACGACAAGaagccggtcatagcctAAAAAAAAAAAAAAAAAAAAAAA*A*A |
| 1471 | |
| SEQ ID: | ATGGACGACGACGACAAGcatcagtaggtgacgcAAAAAAAAAAAAAAAAAAAAAAA*A*A |
| 1472 | |
| SEQ ID: | ATGGACGACGACGACAAGaatcggcgcattgggaAAAAAAAAAAAAAAAAAAAAAAA*A*A |
| 1473 | |
| SEQ ID: | ATGGACGACGACGACAAGgaaattgaggtcctgcAAAAAAAAAAAAAAAAAAAAAAA*A*A |
| 1474 | |
| SEQ ID: | ATGGACGACGACGACAAGacctgcgtgactcttgAAAAAAAAAAAAAAAAAAAAAAA*A*A |
| 1475 | |
| SEQ ID: | ATGGACGACGACGACAAGgcgcgggtaatcatacAAAAAAAAAAAAAAAAAAAAAAA*A*A |
| 1476 | |
| SEQ ID: | ATGGACGACGACGACAAGtcttaggctttcgtgcAAAAAAAAAAAAAAAAAAAAAAA*A*A |
| 1477 | |
| SEQ ID: | ATGGACGACGACGACAAGccgaagacactgtcgtAAAAAAAAAAAAAAAAAAAAAAA*A*A |
| 1478 | |
| SEQ ID: | ATGGACGACGACGACAAGtcatttccccgcctctAAAAAAAAAAAAAAAAAAAAAAA*A*A |
| 1479 | |
| SEQ ID: | ATGGACGACGACGACAAGccttgtgcgtatgtaaAAAAAAAAAAAAAAAAAAAAAAA*A*A |
| 1480 | |
| SEQ ID: | ATGGACGACGACGACAAGtgcgttggtctaaaggAAAAAAAAAAAAAAAAAAAAAAA*A*A |
| 1481 | |
| SEQ ID: | ATGGACGACGACGACAAGccctactaacaatgtcAAAAAAAAAAAAAAAAAAAAAAA*A*A |
| 1482 | |
| SEQ ID: | ATGGACGACGACGACAAGtcctcttagcttgggcAAAAAAAAAAAAAAAAAAAAAAA*A*A |
| 1483 | |
| SEQ ID: | ATGGACGACGACGACAAGctcttacccgcgataaAAAAAAAAAAAAAAAAAAAAAAA*A*A |
| 1484 | |
| SEQ ID: | ATGGACGACGACGACAAGtctgttgggttgtccgAAAAAAAAAAAAAAAAAAAAAAA*A*A |
| 1485 | |
| SEQ ID: | ATGGACGACGACGACAAGagaagtggtcttagacAAAAAAAAAAAAAAAAAAAAAAA*A*A |
| 1486 | |
| SEQ ID: | ATGGACGACGACGACAAGtcagaacaagtcatgcAAAAAAAAAAAAAAAAAAAAAAA*A*A |
| 1487 | |
| SEQ ID: | ATGGACGACGACGACAAGaatccatcggccagtaAAAAAAAAAAAAAAAAAAAAAAA*A*A |
| 1488 | |
| SEQ ID: | ATGGACGACGACGACAAGtcatcagaagcggaagAAAAAAAAAAAAAAAAAAAAAAA*A*A |
| 1489 | |
| SEQ ID: | ATGGACGACGACGACAAGcgttaggttggactacAAAAAAAAAAAAAAAAAAAAAAA*A*A |
| 1490 | |
| SEQ ID: | ATGGACGACGACGACAAGgattagcatcccgaggAAAAAAAAAAAAAAAAAAAAAAA*A*A |
| 1491 | |
| SEQ ID: | ATGGACGACGACGACAAGtacctgaatagtcacgAAAAAAAAAAAAAAAAAAAAAAA*A*A |
| 1492 | |
| SEQ ID: | ATGGACGACGACGACAAGagaaccgcatgtcaccAAAAAAAAAAAAAAAAAAAAAAA*A*A |
| 1493 | |
| SEQ ID: | ATGGACGACGACGACAAGcgattcatatggaccgAAAAAAAAAAAAAAAAAAAAAAA*A*A |
| 1494 | |
| SEQ ID: | ATGGACGACGACGACAAGgaacgaggcctattgtAAAAAAAAAAAAAAAAAAAAAAA*A*A |
| 1495 | |
| SEQ ID: | ATGGACGACGACGACAAGtgggagatatgtaaccAAAAAAAAAAAAAAAAAAAAAAA*A*A |
| 1496 | |
| SEQ ID: | ATGGACGACGACGACAAGttctgaaaacgaagccAAAAAAAAAAAAAAAAAAAAAAA*A*A |
| 1497 | |
| SEQ ID: | ATGGACGACGACGACAAGagtctctttatgacccAAAAAAAAAAAAAAAAAAAAAAA*A*A |
| 1498 | |
| SEQ ID: | ATGGACGACGACGACAAGgagctagtaagacgccAAAAAAAAAAAAAAAAAAAAAAA*A*A |
| 1499 | |
| SEQ ID: | ATGGACGACGACGACAAGaccggtccttcgactaAAAAAAAAAAAAAAAAAAAAAAA*A*A |
| 1500 | |
| SEQ ID: | ATGGACGACGACGACAAGaaatgacgggcgtcacAAAAAAAAAAAAAAAAAAAAAAA*A*A |
| 1501 | |
| SEQ ID: | ATGGACGACGACGACAAGtctcggacccaatcctAAAAAAAAAAAAAAAAAAAAAAA*A*A |
| 1502 | |
| SEQ ID: | ATGGACGACGACGACAAGccatggatcaaaggccAAAAAAAAAAAAAAAAAAAAAAA*A*A |
| 1503 | |
| SEQ ID: | ATGGACGACGACGACAAGtcggtatgtgaatcccAAAAAAAAAAAAAAAAAAAAAAA*A*A |
| 1504 | |
| SEQ ID: | ATGGACGACGACGACAAGggttcatgatcgtatcAAAAAAAAAAAAAAAAAAAAAAA*A*A |
| 1505 | |
| SEQ ID: | ATGGACGACGACGACAAGtaagattctccccttcAAAAAAAAAAAAAAAAAAAAAAA*A*A |
| 1506 | |
| SEQ ID: | ATGGACGACGACGACAAGaaatctaactgccgtgAAAAAAAAAAAAAAAAAAAAAAA*A*A |
| 1507 | |
| SEQ ID: | ATGGACGACGACGACAAGtactgatcatttccgcAAAAAAAAAAAAAAAAAAAAAAA*A*A |
| 1508 | |
| SEQ ID: | ATGGACGACGACGACAAGgtaggatcacggcgttAAAAAAAAAAAAAAAAAAAAAAA*A*A |
| 1509 | |
| SEQ ID: | ATGGACGACGACGACAAGcttgatgtcgtcaatcAAAAAAAAAAAAAAAAAAAAAAA*A*A |
| 1510 | |
| SEQ ID: | ATGGACGACGACGACAAGggaagtctagcgagtcAAAAAAAAAAAAAAAAAAAAAAA*A*A |
| 1511 | |
| SEQ ID: | ATGGACGACGACGACAAGtctctgctcgaggagtAAAAAAAAAAAAAAAAAAAAAAA*A*A |
| 1512 | |
| SEQ ID: | ATGGACGACGACGACAAGctttgcacgagagccaAAAAAAAAAAAAAAAAAAAAAAA*A*A |
| 1513 | |
| SEQ ID: | ATGGACGACGACGACAAGactttaccaatggcgaAAAAAAAAAAAAAAAAAAAAAAA*A*A |
| 1514 | |
| SEQ ID: | ATGGACGACGACGACAAGgcagaatagcgactcgAAAAAAAAAAAAAAAAAAAAAAA*A*A |
| 1515 | |
| SEQ ID: | ATGGACGACGACGACAAGcgaacgttgcgtttggAAAAAAAAAAAAAAAAAAAAAAA*A*A |
| 1516 | |
| SEQ ID: | ATGGACGACGACGACAAGtgaagtctcgaagtgaAAAAAAAAAAAAAAAAAAAAAAA*A*A |
| 1517 | |
| SEQ ID: | ATGGACGACGACGACAAGcccttgggcataaaacAAAAAAAAAAAAAAAAAAAAAAA*A*A |
| 1518 | |
| SEQ ID: | ATGGACGACGACGACAAGggctagcagttgagtgAAAAAAAAAAAAAAAAAAAAAAA*A*A |
| 1519 | |
| SEQ ID: | ATGGACGACGACGACAAGatgggctatggtggtaAAAAAAAAAAAAAAAAAAAAAAA*A*A |
| 1520 | |
| SEQ ID: | ATGGACGACGACGACAAGtaccactaggaatcagAAAAAAAAAAAAAAAAAAAAAAA*A*A |
| 1521 | |
| SEQ ID: | ATGGACGACGACGACAAGacataggggcattgagAAAAAAAAAAAAAAAAAAAAAAA*A*A |
| 1522 | |
| SEQ ID: | ATGGACGACGACGACAAGgttcatagatagcgcaAAAAAAAAAAAAAAAAAAAAAAA*A*A |
| 1523 | |
| SEQ ID: | ATGGACGACGACGACAAGtggctttcctaacagcAAAAAAAAAAAAAAAAAAAAAAA*A*A |
| 1524 | |
| SEQ ID: | ATGGACGACGACGACAAGgaagcgtccatatgacAAAAAAAAAAAAAAAAAAAAAAA*A*A |
| 1525 | |
| SEQ ID: | ATGGACGACGACGACAAGcacaagcgactctttcAAAAAAAAAAAAAAAAAAAAAAA*A*A |
| 1526 | |
| SEQ ID: | ATGGACGACGACGACAAGaagatattccgcgtgcAAAAAAAAAAAAAAAAAAAAAAA*A*A |
| 1527 | |
| SEQ ID: | ATGGACGACGACGACAAGgtccaaatcacaccgtAAAAAAAAAAAAAAAAAAAAAAA*A*A |
| 1528 | |
| SEQ ID: | ATGGACGACGACGACAAGgacgtcatcgtacctgAAAAAAAAAAAAAAAAAAAAAAA*A*A |
| 1529 | |
| SEQ ID: | ATGGACGACGACGACAAGacagctgctgtgcatcAAAAAAAAAAAAAAAAAAAAAAA*A*A |
| 1530 | |
| SEQ ID: | ATGGACGACGACGACAAGttgtaacagtgcaacgAAAAAAAAAAAAAAAAAAAAAAA*A*A |
| 1531 | |
| SEQ ID: | ATGGACGACGACGACAAGagctgttatgcgccgtAAAAAAAAAAAAAAAAAAAAAAA*A*A |
| 1532 | |
| SEQ ID: | ATGGACGACGACGACAAGttgcccaaaaccctgtAAAAAAAAAAAAAAAAAAAAAAA*A*A |
| 1533 | |
| SEQ ID: | ATGGACGACGACGACAAGagctaagtcgctggtaAAAAAAAAAAAAAAAAAAAAAAA*A*A |
| 1534 | |
| SEQ ID: | ATGGACGACGACGACAAGtcctgtaattacgcctAAAAAAAAAAAAAAAAAAAAAAA*A*A |
| 1535 | |
| SEQ ID: | ATGGACGACGACGACAAGcgcctgatcctttgagAAAAAAAAAAAAAAAAAAAAAAA*A*A |
| 1536 | |
| SEQ ID: | ATGGACGACGACGACAAGacctctgtcgagttacAAAAAAAAAAAAAAAAAAAAAAA*A*A |
| 1537 | |
| SEQ ID: | ATGGACGACGACGACAAGgacgttgtagcaggatAAAAAAAAAAAAAAAAAAAAAAA*A*A |
| 1538 | |
| SEQ ID: | ATGGACGACGACGACAAGatggctcaacgaggagAAAAAAAAAAAAAAAAAAAAAAA*A*A |
| 1539 | |
| SEQ ID: | ATGGACGACGACGACAAGagaggtacatgagaggAAAAAAAAAAAAAAAAAAAAAAA*A*A |
| 1540 | |
| SEQ ID: | ATGGACGACGACGACAAGtgacagcccatctcgtAAAAAAAAAAAAAAAAAAAAAAA*A*A |
| 1541 | |
| SEQ ID: | ATGGACGACGACGACAAGtgacaacgccatgtctAAAAAAAAAAAAAAAAAAAAAAA*A*A |
| 1542 | |
| SEQ ID: | ATGGACGACGACGACAAGgggttacaacgtatagAAAAAAAAAAAAAAAAAAAAAAA*A*A |
| 1543 | |
| SEQ ID: | ATGGACGACGACGACAAGcatacgatcacggacgAAAAAAAAAAAAAAAAAAAAAAA*A*A |
| 1544 | |
| SEQ ID: | ATGGACGACGACGACAAGtaccccggctatcaacAAAAAAAAAAAAAAAAAAAAAAA*A*A |
| 1545 | |
| SEQ ID: | ATGGACGACGACGACAAGatgaaactcaccgcaaAAAAAAAAAAAAAAAAAAAAAAA*A*A |
| 1546 | |
| SEQ ID: | ATGGACGACGACGACAAGcctatatccattcctgAAAAAAAAAAAAAAAAAAAAAAA*A*A |
| 1547 | |
| SEQ ID: | ATGGACGACGACGACAAGtagcattaacagcgtgAAAAAAAAAAAAAAAAAAAAAAA*A*A |
| 1548 | |

Libraries are then prepared from the digested products using a modified Nextera® XT protocol in which custom primers designed to enrich 3′ end are used. The libraries are then sequenced using an ILLUMINA® platform. Gene expression can then be analyzed by determining the total amount of each of the RNAs present, for each cellular barcode present.

The present methods provide several advantages over previous methods. For example, by using a 384-well PCR plate the reaction volume is decreased (e.g., the volume decreased from 10 μL to 5 μL for reverse transcription and from 25 μL to 10 μL for PCR). Further, by using a restriction enzyme, the current method allows for recovery of about 80-90%, such as 85%, 3′ end sequences that have cell barcode information; a much higher recovery rate compared with other 3′ end selection methods (Table 11).

### VI. Single Cell Gene Expression Analysis, Single Cell RNA Sequencing, and DNA-Labeled Antibody Sequencing

The present methods for the generation of peptide antigens by IVTT using synthesized oligo nucleotides as the template, which are then loaded to MHC monomers and form DNA-BC pMHC tetramers to stain and sort T cells, can also be combined with single cell gene expression analysis platforms, such as BD BD Rhapsody™ Single-Cell Analysis System, or single cell RNA sequencing (scRNA-seq) platforms, such as 10× genomics Chromium or 1CellBio inDrop or Dolomite Bio Nadia. In addition, methods described here can be combined with DNA-labeled antibody sequencing, such as CITE-seq or REAP-seq (Stoeckius et al., 2017) or the commercially available DNA-labeled antibodies, such as BD Ab-seq products or Biolegend TotalSeq (FIGS. 23-28, Table 1). The method that includes the TetTCR-Seq, single cell gene expression or scRNA-seq, and DNA-labeled antibody sequencing is referred to herein as TetTCR-SeqHD.

TetTCR-SeqHD methods described here can use peptide encoding oligos desgined in the TetTCR-Seq or peptide encoding oligos with poly A tail added to the 3′end to interface with scRNA-seq protocols that high-throughput scRNA-seq platforms use. A DNA linker oligonucleotide may be used to covalently linked to streptavidin in order to complementary bind peptide-encoding DNA oligonucleotide. This design makes it possible for only annealing to be required to link the peptide-encoding DNA oligonucleotide to the streptavidin. MID or UMI and cell barcodes from high-throught platforms during reverse transcription may be used. Reverse transcription using primers containing polyT in above single cell analysis platforms can generate cDNA of peptide-encoding DNA oligonucleotide for each individual cell. Reverse transcription part of TetTCR-SeqHD is compatible with single cell RNA sequencing protocols, such as Smart-seq and Smart-seq2 protocols (Ramskold et al., 2012).

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

PE/APC-labeled streptavidin conjugation to DNA Linker—Conjugation of a DNA linker comprising a MID sequence (Table 1) to Phycoerythrin (PE)- and Allophycocyanin (APC)-labeled streptavidin was performed following manufacturer's protocols (SoluLink®). Excess unconjugated DNA linker was removed by 6 wash steps in a Vivaspin® 6 100 kDa protein concentrator (GE® Healthcare). Conjugates were concentrated to ˜120 μl, and then passed through a 0.2 μm centrifugal filter. The molar DNA:protein conjugation ratio was kept between 1:3 to 1:7.

DNA:protein conjugation ratio was determined by absorbance using a 1 mg/ml of PE or APC-labeled streptavidin reference solution. The absorbance of the DNA-streptavidin conjugate was then compared with this standard curve to determine the effective protein concentration of the conjugate. The DNA concentration was determined from the difference in the A260 absorbance between the DNA-streptavidin conjugate and a protein concentration-matched version of the PE/APC streptavidin.

Overlap extension of the DNA-streptavidin conjugate—Annealing of DNA template to DNA-streptavidin conjugate was done at 55° C. for 5 minutes, then cooled to 25° C. at −0.1° C./s in the presence of 250 μM dNTP in 1× CutSmart® buffer (NEB®). Then, 1 μl of extension mixture consisting of 0.1 μl CutSmart® 10×, and 0.125 μl Klenow Fragment Exo-(5 U/ul, NEB) was added before starting the extension at 37° C. for 1 hour. The reaction is stopped by adding EDTA. The extended DNA-streptavidin conjugate was stored at 4° C. These steps correspond to steps 2.1 and 2.2 in FIG. 1A.

In vitro transcription/translation—Peptide-encoding DNA templates were purchased from IDT and SIGMA-ALDRICH®. DNA templates were amplified in a 10 μl PCR reaction with 400 μM dNTP, 1 μM IVTT forward primer (Table 1), 1.05 μM IVTT reverse primer (Table 1), 25 μM DNA template, and 0.0375 U/μl TaKaRa Ex Taq® HS DNA Polymerase (TAKARA BIO USA®). The reaction proceeded for 95° C. 3 min, then 30 cycles of 95° C. 20 s, 52° C. 40 s, 72° C. 45 s, then 72° C. 5 min. The PCR product was diluted with 73.3 μl of water. Corresponds to step 1.1 in FIG. 1A.

20 μl of 1.5× concentrated PUREXPRESS® IVTT master mix (NEW ENGLAND BIOLABS®) consists of 10 μl Solution A, 7.5 μl solution B, 0.8 μl of Release Factor 1+2+3 (5 reaction/μl, NEB special order), 0.25 μl enterokinase (16 U/μl, NEB), 0.25 μl Murine RNase Inhibitor (40 U/ul, NEB), and 1.2 μl H₂O. 1 μl of the diluted PCR product was added to 2 μl of the IVTT master mix on ice and then incubated at 30° C. for 4 hours. This step corresponds to step 1.2 in FIG. 1A.

pMHC UV exchange and tetramerization—pMHC UV exchange and tetramerization follows previously described protocol (Rodenko et al., Yu et al., 2015). The UV exchange was performed for 60 minutes on ice, and then incubated at 4° C. for at least 12 hours. Extended DNA-streptavidin conjugate was then added to its corresponding UV-exchanged pMHC monomer mix at molar ratio of 1:6.7 and incubated at 4° C. for 1 hour to generate DNA pMHC tetramers. This step corresponds to step 1.3 in FIG. 1A.

DNA pMHC tetramer pooling—500 μl of staining buffer (PBS, 5 mM EDTA, 2% FBS, 100 ug/ml salmon sperm DNA, 100 uM d-biotin, 0.05% sodium azide) was added to a 100 kDa VIVASPIN® protein concentrator (GE®) and incubated for at least 30 minutes. The concentrator is spun at 10,000 g and further staining buffer is added until 1 ml of solution have run through the membrane. Immediately prior to cell staining, 0.65 μl of each DNA pMHC tetramer is added to 400 μl of staining buffer, transferred to the concentrator, and then spun at 7,000 g for 10 minutes or longer until the volume reaches ˜50 μl.

DNA pMHC tetramer staining and sorting of T cells—Human Leukocyte Reduction System (LRS) chambers were obtained from de-identified donors by staff members at We Are Blood. The use of LRS chamber from de-identified donors for this study was approved by the Institutional Review Board of the University of Texas at Austin and was complied with all ethical regulations. CD8⁺ T cell isolation was performed following a previously established protocol (Yu et al., 2015).

Cells were resuspended into staining buffer containing ˜60 nM of each DNA-BC pMHC tetramer and 0.025 mg/ml of BV785-CD8a (RPA-T8) antibody and incubated for 1 hour at 4° C. In experiments 1 and 2, a HCV-KLV(WT) binding clone was pre-stained with BV605-CD8a and then spiked into the main sample. Tetramer enrichment was performed either on ice or at 4° C. following published protocol (Yu et al., 2015).

The enriched fraction was eluted off the column and washed into FACS buffer with 0.05% sodium azide, and stained with AF488-CD3, 7-AAD, BV421-CCR7, BV510-CD45RA, and BV785-CD8a (Biolegend). Single cells were sorted using BD FACSARIA™ II into 4 μl lysis buffer following previously published protocol (Zhang et al., 2016).

T cell receptor and DNA-BC sequencing library preparation—Single cell TCR amplification and sequencing was done following published protocol with a minor modification (Zhang et al., 2016). During the first PCR amplification, primers P1 and P2 (SEQ ID NOs: 4-5) were included in the primer mix at 100 nM final concentration for concurrent amplification of TCR and the DNA-BC from the DNA pMHC tetramer (Table 2).

1 μl of first PCR product from the TCR and DNA-BC amplification was combined with 100 nM of a V1f_rxn2 primer (Table 1) and 100 nM of a V1r_rxn2 primer from Table 1, and 0.025 U/μl TAKARA EX TAQ® HS (TAKARA BIO USA®) to 5 μl volume for a second PCR. PCR proceeded at 95° C. 3 minutes, then 10 cycles of 95° C. 20 sec, 55° C. 40 sec, and 72° C. 45 sec, then 72° C. 5 min. These PCR primers include cell barcodes to discriminate between wells, and include partial Illumina adaptor as previously described (Zhang et al., 2016).

A third PCR was used to add the remaining ILLUMINA® sequencing adaptors using ILLU_f and ILLU_r primers (Table 1). This PCR was identical to that of the prior, except that it only used 5 cycles. Multiple wells are then pooled and purified by gel electrophoresis and gel extraction. Libraries were sequenced on the ILLUMINA® MISEQ® using the V2 kit. The libraries were sequenced to a depth of at least 6000 reads/cell.

DNA-BC sequence processing—Raw reads were filtered based on the constant region of the DNA-BC. Reads were further separated according to cell barcodes. Within each cell barcode, reads with an identical MID sequence were clustered together and a consensus peptide-encoding sequence was built for each cluster. Each cluster represents one MID count.

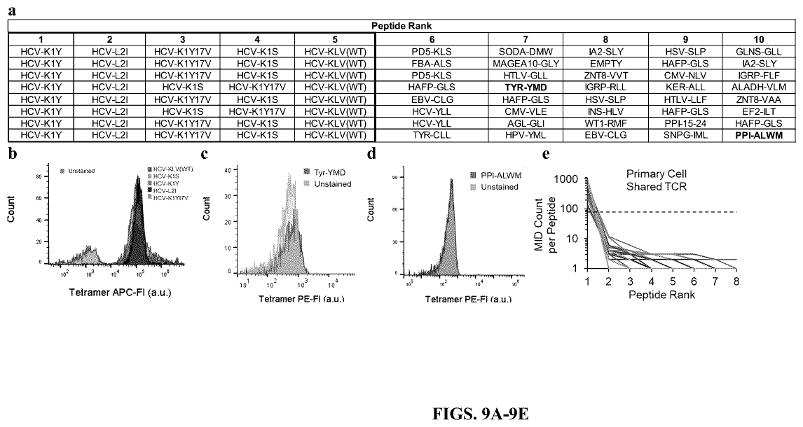

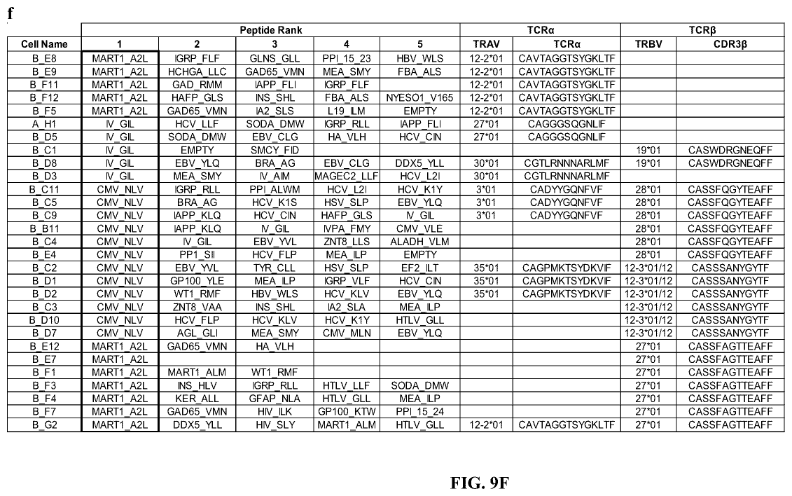

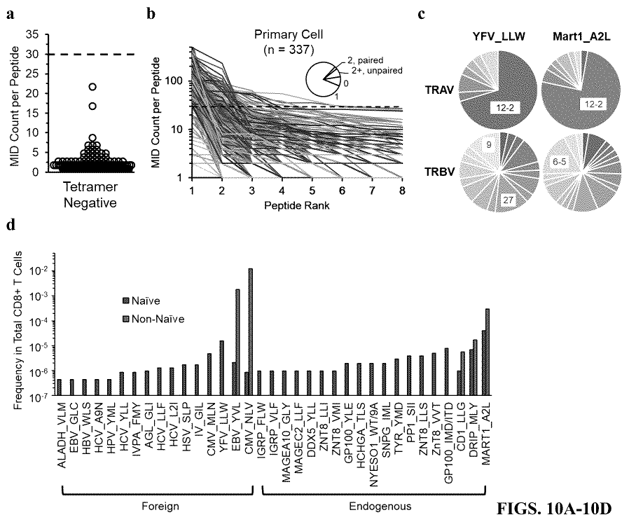

Clusters were filtered based on the peptide-encoding region to be 25-30 nt in length, and with a Levenshtein distance no greater than 2 from the nearest known DNA-BC sequence. A histogram was then created expressing the % of total reads belonging to each group of clusters sharing the same read count. Low read count clusters, which occur due to sequencing errors, were removed (FIG. 9) (Fu et al., 2014). The clusters are then collected into their corresponding cell and peptide based on the cell barcode and peptide-encoding DNA sequence, respectively.

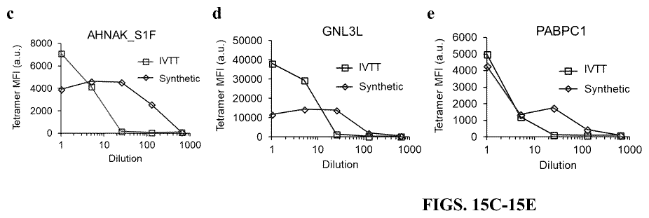

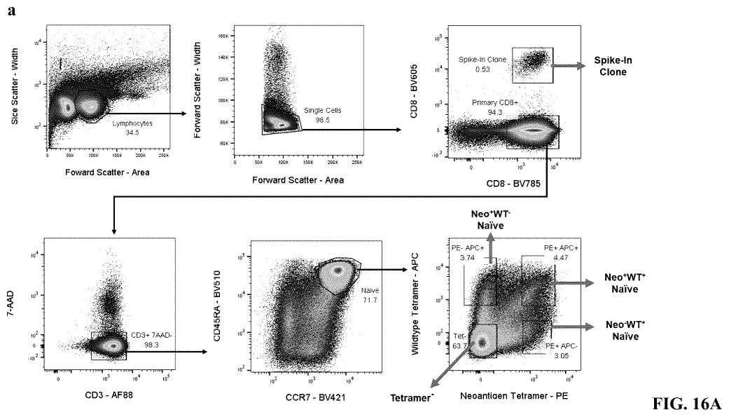

Calculation of percent cross-reactive T cells for Experiment 3-6: The relative proportion of T cells belonging to the Neo⁺WT⁺, Neo⁻WT⁺, and Neo⁺WT⁻ antigen-binding cell populations was calculated for each Neo-WT antigen pair using cells with positive antigen detection. The analysis was restricted to cells with the one identified antigen in the Neo⁻WT⁺ and Neo⁺WT⁻ sorted populations and the two identified antigens in the Neo⁺WT⁺ sorted population (FIGS. 113E, 15E, 18I). From this dataset, normalization was performed to account for differences in the frequency and number of cells sorted for the three cell populations. Taking these two normalizations into account, the equation for calculating the relative proportion p of cells binding to peptide a in population b for Experiment 3-4 is:

$$p ( {a}_{i} , {b}_{j} ) = \frac{relfreq ( {b}_{j} ) * \frac{count ( {a}_{i} , {b}_{j} )}{totalsort ( {b}_{j} )}}{{\sum }_{b} \frac{relfreq ( b ) count ( {a}_{i} , b )}{totalsort ( b )}}$$

aᵢ refers to a Neo-WT antigen pair in the Neo⁺WT⁺ population, corresponding WT peptide only in the Neo⁻WT⁺ population, and corresponding Neo peptide only in the Neo⁺WT⁻ population. bⱼ refers to one of the three cell populations Neo⁺WT⁻, Neo⁻WT⁺, or Neo⁺WT⁺. count(aᵢ,bⱼ) refers to the antigen-binding T cell count in cell population bⱼ binding to peptide aᵢ. Relfreq(bⱼ) refers to the percentage of cell population bⱼ taken from the tetramer gating in the tetramer-enriched fraction, which is a measure of the relative cell frequency (FIG. 112A). totalsort(bⱼ) is the total number of cells sorted for cell population bⱼ.

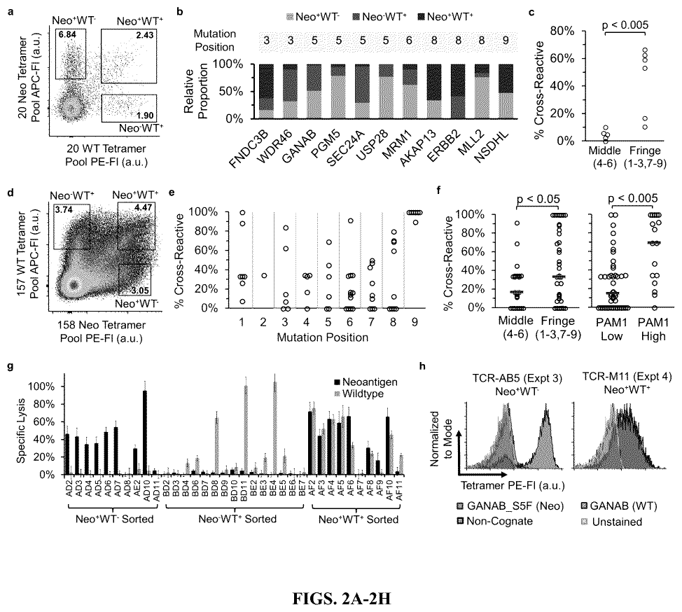

The percent cross reactive T cells for any Neo-WT antigen pair aᵢ is simply p(aᵢ,b_{Neo+WT+}) (same values as red bars in FIG. 2B). While this calculation can be performed for all Neo-WT antigen pairs, the analysis was restricted to Neo-WT antigen pairs containing at least 3 cells where both the Neo and WT antigen were detected in at least one cell.

An aggregate analysis was performed for experiment 5-6. Since cells are aggregated from these two experiments, the cell counts were normalized in the three Tetramer⁺ populations but not the cell frequency because the relative frequency of the three cell populations in both experiments were comparable between one another. The altered equation used for Experiment 5-6 is the following:

$$p ( {a}_{i} , {b}_{j} ) = \frac{count ( {a}_{i} , {b}_{j} ) / totalsort ( {b}_{j} )}{{\sum }_{{b}_{1}}^{{b}_{3}} \frac{count ( {a}_{i} , b )}{totalsort ( {b}_{j} )}}$$

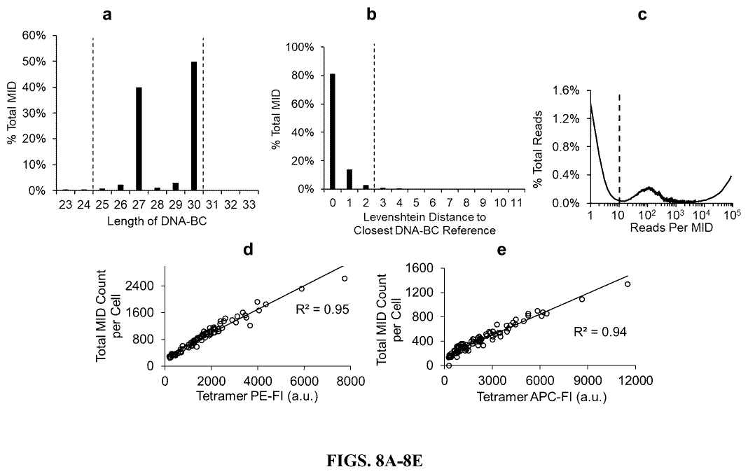

T cell lines and functional assay: T cell lines were generated according to previously published protocol, but using the DNA-BC pMHC tetramer pool. Cells were gated in the same manner as FIG. 8 except for the AF488 channel, where CD3-AF488 was replaced by the dump channel CD4,14,16,19,32,56-AF488. 5 cells from the same population (Neo⁺WT⁻, Neo⁻WT⁺, Neo⁺WT⁺) were sorted into each well. Functional status was analyzed 10-21 days after re-stimulation.

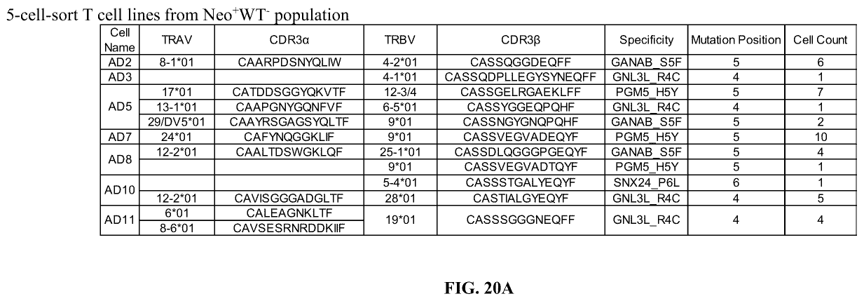

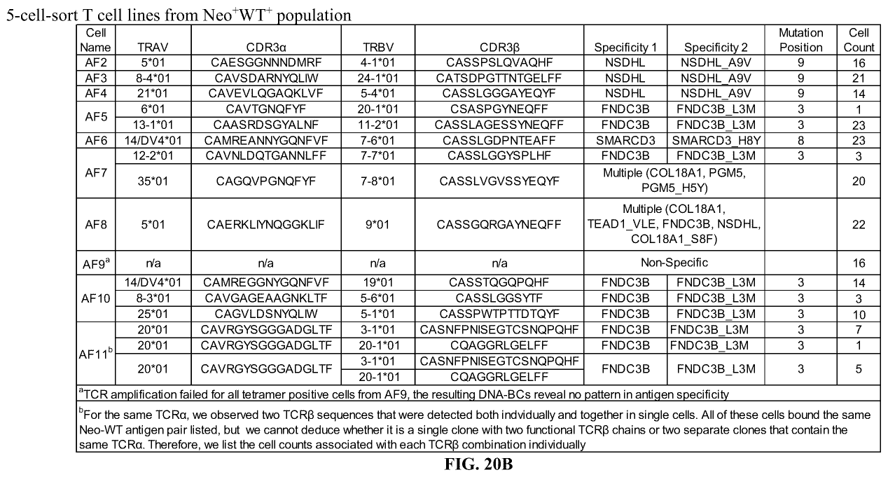

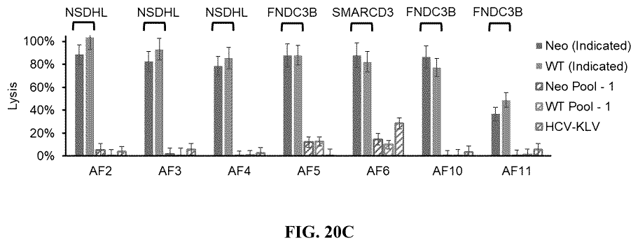

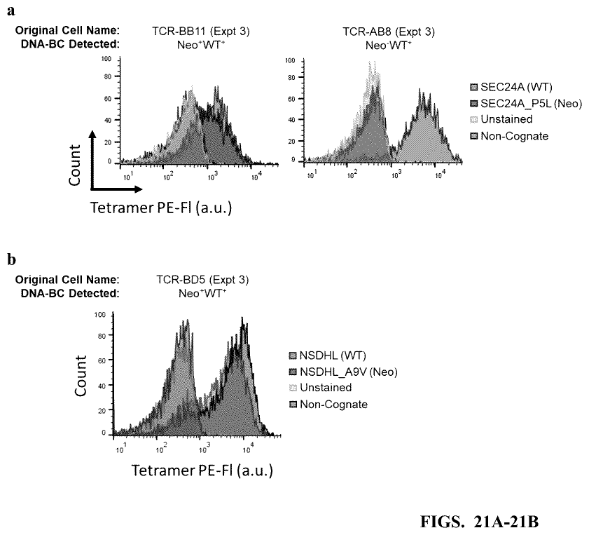

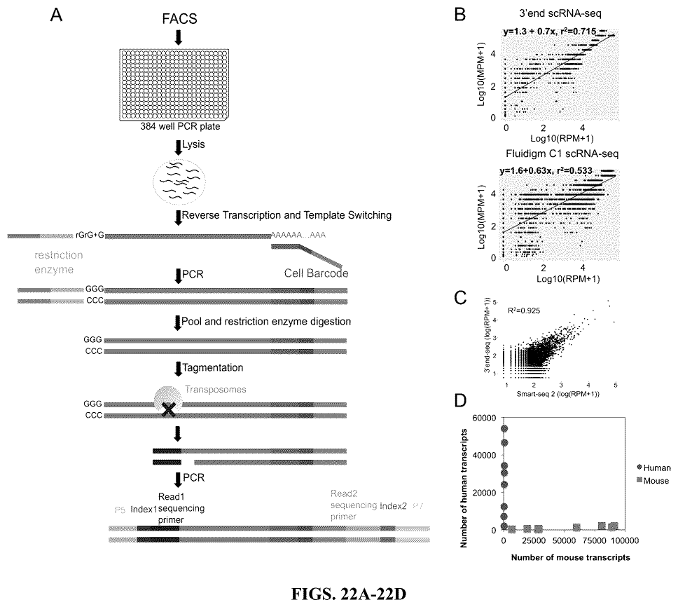

Functionality was measured and analyzed using the LDH cytotoxicity assay kit (Thermofisher) following manufacturer's instructions as described previously. For FIG. 2G and FIG. 20, T2 cells (ATTC) were pulsed with a peptide pool consisting of either the 20 neoantigen peptides (250 mM total, 12.5 mM each peptide) or 20 wildtype peptides (250 mM total, 12.5 mM each peptide). Background cytotoxicity was subtracted by using T2 cells pulsed with HCV-KLV(WT) peptide (250 mM). For FIG. 21C, T2 cells were pulsed with 12.5 mM of a single peptide or a peptide pool consisting of the 19 indicated neo-antigen or WT peptides at 12.5 mM per peptide. Background cytotoxicity was subtracted by using T2 cells not pulsed with peptide. For each well, 60,000 T cells were incubated with 6,000 peptide-pulsed T2 cells for 4 hours at 37° C. Each condition for each cell line (derived from 5 single sorted cells) was performed in triplicates.

Lentiviral TCR transduction: Lentivirus production and TCR transduction was performed as previously described with the following modifications. TCR were synthesized as GenParts (GenScript) and was cloned into pLEX_307 (a gift from David Root via Addgene) under EF-1a promoter. The vector also confers puromycin resistance. All vector sequences were confirmed via Sanger sequencing prior to viral production. 72 hours after transduction, expression of the TCR was analyzed by flow cytometry. Antigen binding of the transduced cells was confirmed by pMHC tetramer and anti-CD3 antibody (Biolegend) staining.

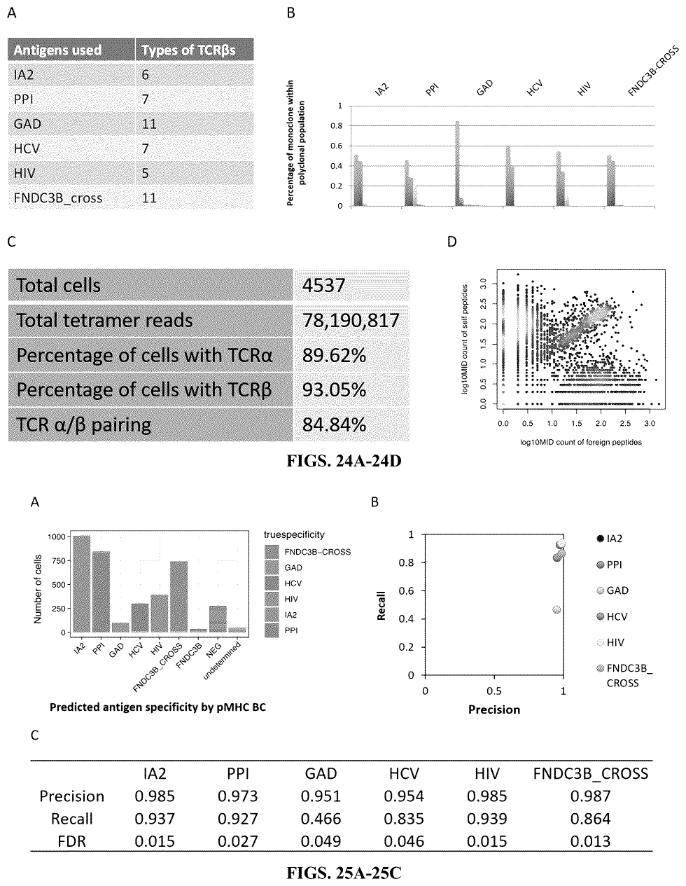

Criteria for peptide classification: MID threshold and signal-to-noise ratio: In order to characterize the non-specific binding level of DNA-BC peptides to T cells, a peptide was defined to be positively binding if the fluorescence intensity of the corresponding pMHC tetramer is above background level, which is set using the flow through fraction after tetramer enrichment. To measure background, fluorescent tetramer negative (Tetramer) single CD8+ T cells were sorted from the tetramer enriched fraction and measured the number of MIDs associated with each of the non-specifically bound peptides. Results show that these non-specific bound DNA-BCs from Tetramer single cells have low MID counts associated with each peptide (FIG. 1D, 13A, 15A, 18A, 18E). Another version of peptide classification is based on MID distribution (FIG. 24D, 27A-B).

The first criteria that was applied to detect positively bound peptides from background level of non-specific binding is a MID count threshold. This threshold was defined to be the maximum MID count-per-peptide from the Tetramer population with an added 25% buffer, rounded to the nearest tens digit (dashed lines in FIG. 1D, 13A, 15A, 18A, 18E). This value was determined for each TetTCR-Seq experiment.

The second criteria used for each cell was a signal-to-noise ratio between two borderline peptides, which is defined to be the ratio of the peptide with the lowest MID count above the MID threshold to the peptide with the highest MID count below the MID threshold. The spike-in clone from Experiment 1 was used as the positive control for the MID counts associated with positive and negatively binding peptides, which was validated using traditional tetramer staining (FIG. 1E, 1F, 10A-D). By aggregating all cells from this spike-in clone, the signal-to-noise ratio ranged from 3.6:1 to 61:1. Using this as a guide, the signal-to-noise ratio was set to be greater than 2:1; Cells with a signal-to-noise ratio below this threshold was removed from analysis because the segregation in MID counts between positive and negative binding peptides was too low.

### Example 2

### Establishment of TetTCR-Seq

To address the challenges associated with prior approaches to TCR analysis, Tetramer Associated TCR Sequencing (TetTCR-Seq) was developed. TetTCR-Seq is a platform for high-throughput pairing of TCR sequence with potentially multiple antigenic pMHC species at single T cell resolution. First, a large library of fluorescently labeled, DNA-barcoded (DNA-BC) pMHC tetramers was constructed in an inexpensive and rapid manner using in vitro transcription/translation (IVTT) (FIG. 1A). Next, tetramer-stained cells were single-cell sorted for concurrent amplification of the DNA-BC and TCRαβ genes in RT-PCR (FIG. 1B). These amplicons were further PCR amplified separately in parallel wells to add the cell barcode and sequencing adapters. A molecular identifier (MID) consisting of 12 random nucleotides (nt) was included in the DNA-BC to provide absolute counting of the copy number for each species of tetramers bound to the cell. Finally, the linking of multiple peptide specificities with their bound TCRα and TCRβ sequences was done using predetermined nucleotide-based cell barcodes. DNA-BC pMHC tetramers are compatible with magnetic enrichment methods for the isolation of rare antigen-binding precursor T cells, making TetTCR-Seq a versatile platform to analyze both clonally expanded and precursor T cells.

To construct large pMHC libraries via UV-mediated peptide exchange using traditional chemically synthesized peptide is costly with long turnaround times. To solve this problem, TetTCR-Seq utilizes a set of peptide-encoding oligonucleotides that serve as both the DNA-BCs for identifying antigen specificities and DNA templates for peptide generation via IVTT (FIG. 1A). Synthesizing 60 length oligonucleotides is less expensive (about 20-fold) and faster (1-2 days instead of weeks) than synthesizing peptides. The IVTT step only adds a few additional hours, making it possible to generate peptide libraries that are tailored to any disease and/or individuals quickly and affordably.

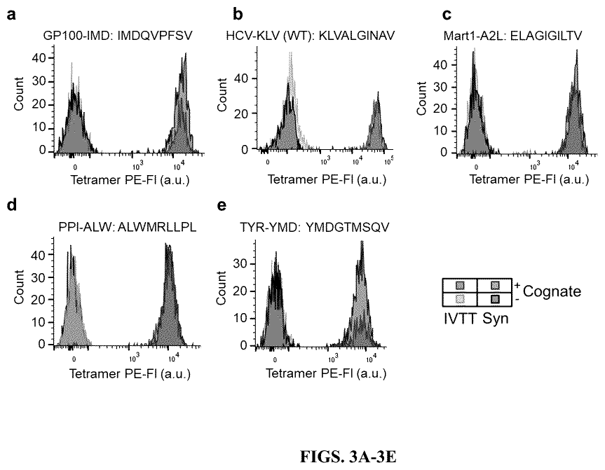

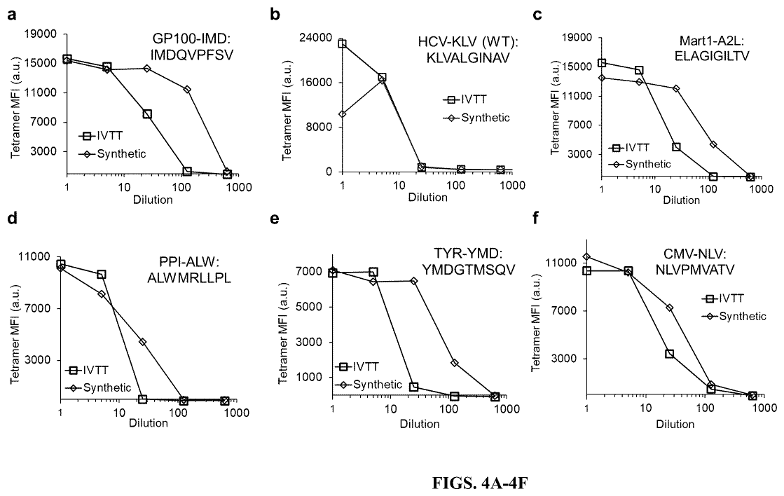

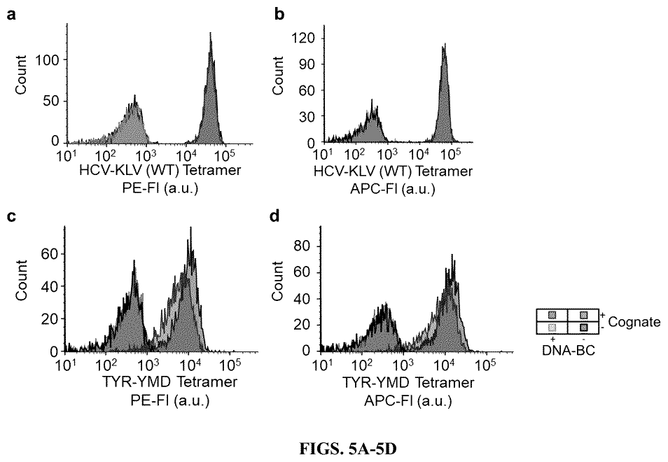

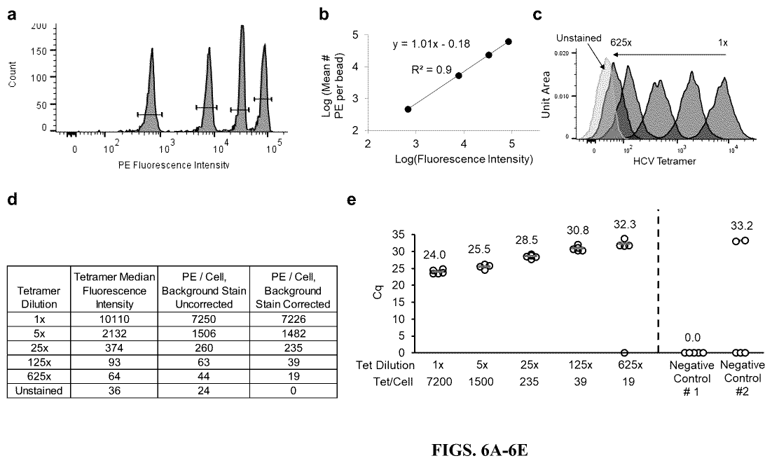

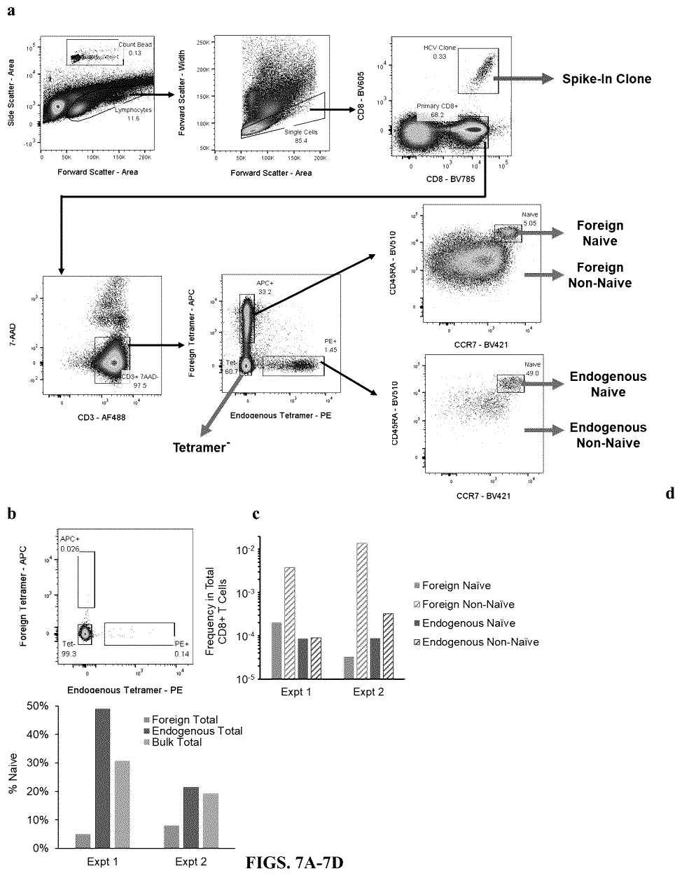

pMHC tetramers generated by UV-exchange using either IVTT- or synthetic-produced peptides stained cognate and non-cognate T cell clones similarly (FIGS. 1C and 3). IVTT can generate 20-100 μM of the desired peptide, which is in the concentration range commonly used for UV-mediated peptide exchange (FIG. 4). Covalent attachment of the DNA-BC to PE or APC streptavidin scaffold did not hinder staining performance of the resulting DNA-BC pMHC tetramer (FIG. 5). DNA-BC pMHC tetramer achieved a detection sensitivity of as few as ˜19 tetramer complexes per cell, which is comparable to the fluorescent pMHC tetramer detection limit (FIG. 6). 6 main TetTCR-Seq experiments were performed and they are summarized in FIG. 7.

The ability of TetTCR-Seq was assessed to accurately link TCRαβ sequence with pMHC binding from primary CD8⁺ T cells in human peripheral blood. In Experiment 1, a 96-peptide library was constructed consisting of well documented foreign and endogenous peptides bound to HLA-A2 and isolated dominant pathogen-specific T cells as well as rare precursor antigen-binding T cells from a healthy CMV sero-positive donor (FIG. 1, 8). To test whether TetTCR-Seq can detect cross-reactive peptides, included in the panel was a documented HCV wildtype (WT) peptide, HCV-KLV(WT), and 4 candidate altered peptide ligands (APL) with 1-2 amino acid (AA) substitutions. A T cell clone that was established using HCV-KLV (WT) was spiked into the donor's sample to test for its potential to cross-react with the APLs.

TCRα and TCRβ sequences were successfully amplified along with the DNA-BC and the efficiencies are comparable to previous protocols (FIG. 7). Sequencing error-containing DNA-BC reads were removed before downstream analysis (FIG. 9A-C). Positively binding peptides were classified by their MID counts using two criteria: an MID threshold derived from tetramer negative controls and a ratio of MID counts between the peptides above and below this threshold (FIG. 1D). MID counts also correlated with the fluorescence staining intensity (FIG. 9D-E), confirming its utility in quantifying the number of bound pMHC tetramers.

Using this classification scheme, the expected HCV-KLV(WT) epitope were identified from all sorted cells belonging to the spike-in clone (FIG. 1E, 10A). In addition, it was discovered that all four APLs were also classified as binders. The 6^{th} ranked peptide and beyond, by MID count, all classified as non-binders; Their MID species varied from cell-to-cell, which suggests non-specific binding. A separate pMHC staining experiment on the T cell clone confirmed that the classification is accurate (FIGS. 1F and 10B-D). It was also confirmed that all primary cells with shared TCR sequences also shared the same peptide specificity (=FIG. 10E-F). These results show that TetTCR-Seq is able to resolve positively binding peptides in primary T cell populations and identify up to five cross-reactive peptides per cell.

The majority of primary T cells were classified as binding one peptide (FIG. 1G). This result is expected because the probability of TCR cross-reactivity between similar peptides is higher than disparate ones, and most of the peptides used in Experiment 1 had a Levenshtein distance of greater than 4 among each other (Table 2, 4). However, two cells were detected that were classified as binding GP100-IMD and GP100-ITD simultaneously (FIG. 1G); these two peptides are only 1 AA apart and cross-reactivity has been previously reported.

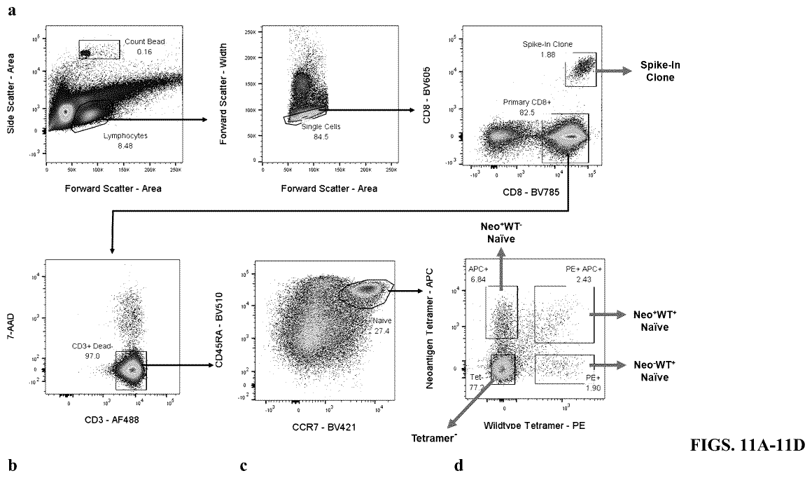

Among the peptides surveyed, a high degree of peptide diversity was found in the foreign-specific naïve T cell repertoire (FIG. 1H). This diversity reduced in the non-naïve repertoire to two dominant peptides for CMV and influenza of high frequency (FIG. 1H). This is expected given the CMV sero-positive status and a high probability of influenza exposure or vaccination for this donor. The majority of cells within the endogenous-binding population responded to MART1-A2L, which corroborates its high documented frequency relative to other endogenous epitopes (FIG. 1H). Linked TCR and DNA-BC analysis uncovered dominant recognition patterns in MART1-A2L and YFV-LLW specific TCRs by the TCRα V gene 12-2 and 12-1/12-2, respectively, with variable TCRβ V gene usage (FIG. 1I). This result is consistent with recent literature reports. In Experiment 2, TetTCR-Seq was performed on a second CMV seropositive donor and verified the findings from Experiment 1 (FIG. 11). These results highlight the ability of TetTCR-Seq to accurately link pMHC binding with TCR sequences.

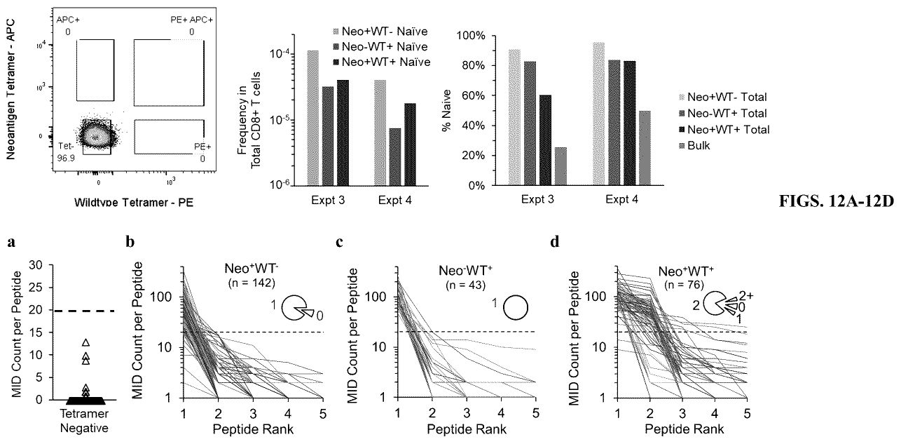

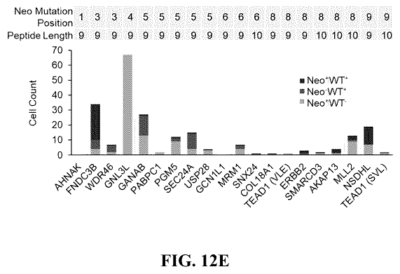

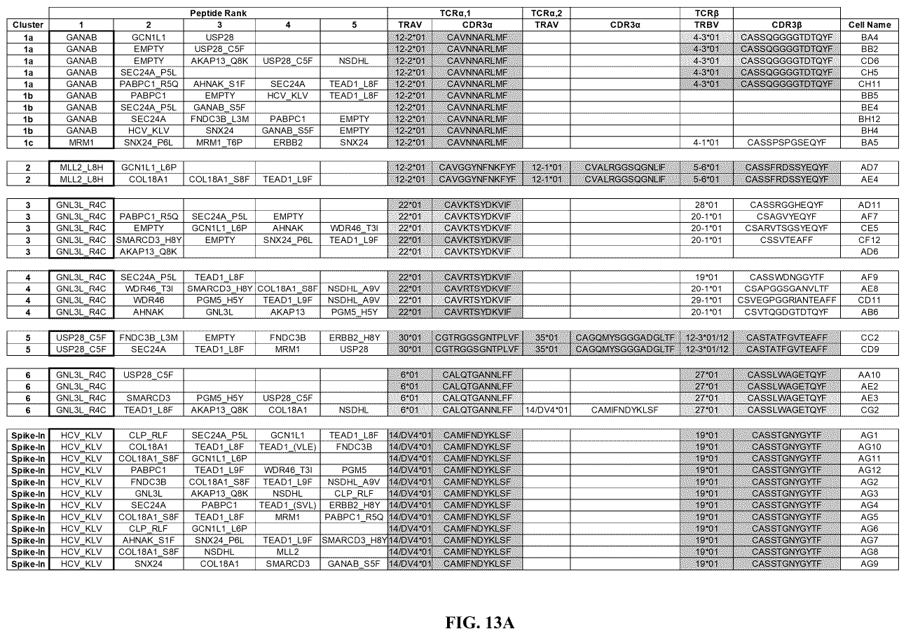

TetTCR-Seq was next applied to profile cancer antigen cross-reactivity in healthy donor peripheral blood T cells and isolate neo-antigen (Neo)-specific TCRs with no cross-reactivity to wildtype counterpart antigen (WT). Naïve T cells from healthy donors are a useful source of Neo-specific TCRs. However, most neo-antigens are 1 AA from the WT sequence, meaning that Neo-specific TCRs can potentially cross-react with endogenous host cells to cause severe autoimmunity, and even death. In Experiment 3, 20 pairs of Neo-WT peptides were surveyed that bind with high affinity to HLA-A2. pMHC tetramer-based selection of naïve T cells has an inherent risk of selecting T cells reactive to peptides that are not naturally processed. As such, peptides were also chosen based on previous evidence of tumor expression and T cell targeting. Neo and WT pMHC pools were labeled using two separate fluorophores, allowing for sorting of three cell populations, Neo⁺WT⁻, Neo⁻WT⁺, and Neo⁺WT⁺ (FIGS. 2A and 12).

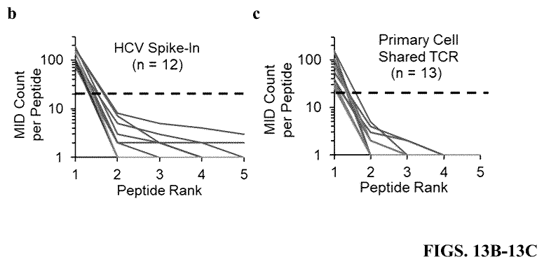

Tetramer⁺ CD8⁺ T cells were enriched in the naive phenotype compared to bulk, indicative of no prior exposure to the surveyed antigens (FIG. 12D). No more than one peptide was detected in T cells sorted from either the Neo⁺WT⁻ or the Neo⁻WT⁺ populations (FIG. 13A-C). T cells with two detected peptide binders accounted for 84% of the Neo⁺WT⁺ population, 98% of which belonged to a Neo-WT antigen pair (FIG. 13D).

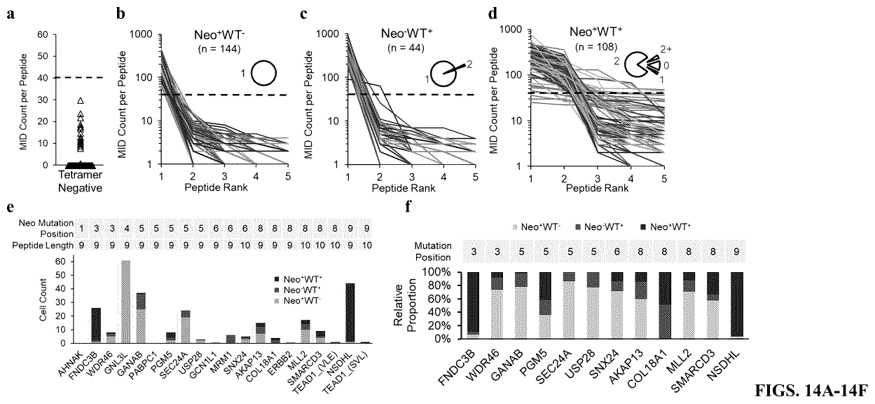

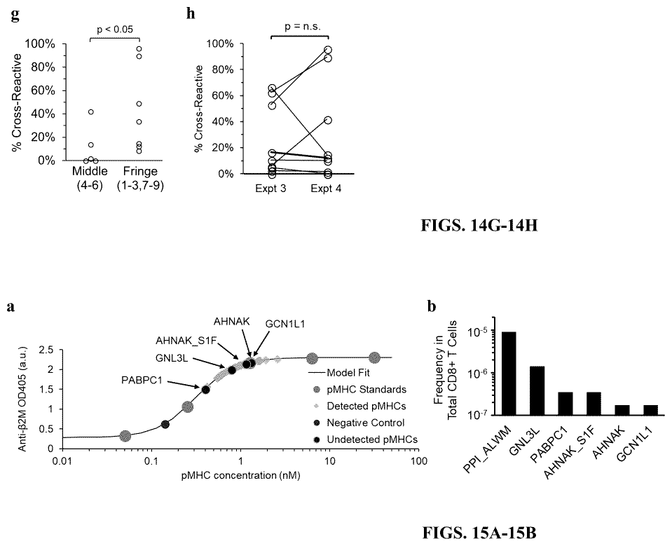

Just as in Experiment 1, the criteria correctly classified all peptides for the spike-in HCV-binding clone (FIG. 14). Interestingly, despite only sorting on the CCR7⁺CD45RA⁺ naïve phenotype, 6 clusters of primary T cells were detected with shared TCR sequences on the AA level (Clusters 1-6 in FIG. 14A). Cells with shared TCR α and β sequences bound the same peptide (Clusters 1a, 2, 5, 6). Many of these TCRs were found to be encoded by different TCRα and TCRβ nucleotide sequences, indicating convergent VDJ recombination. It was also found that in some TCRs, the same TCR α chain is sufficient for them to engage the same pMHC, while TCRβ chains are all different (Clusters 3 and 4). However, in other TCRs, the same TCR α paired with a different TCR β chain can lead to different peptide specificity (Compare Cluster 1c to 1a). These results highlight the advantage of high-throughput linking of TCR sequence with its antigenic peptide as a first step in deciphering the TCR repertoire, which could be complementary to bioinformatics analysis.

Cells in the Neo⁺WT⁺ population bound 11 of the 20 Neo-WT antigen pairs, indicating that Neo-WT cross-reactivity is wide-spread in the precursor T cell repertoire (FIGS. 2B and 13E). By analyzing the proportion of mono and cross-reactive T cells from each Neo-WT pair, it was observed that neo-antigens with mutations at fringe positions 3, 8, and 9 elicited significantly more cross-reactive responses than the ones at center positions 4, 5, and 6 (FIG. 2C). This is consistent with observations made by others using alanine substitutions on peptides in a mouse model. In Experiment 4, TetTCR-Seq was performed on a separate donor and observed the same trend (FIG. 15). The percentage of cross-reactive T cells for the same Neo-WT antigen pair was not significantly different between Experiment 3 and 4, indicating that this property is conserved between donors for the peptides tested (FIG. 15H).

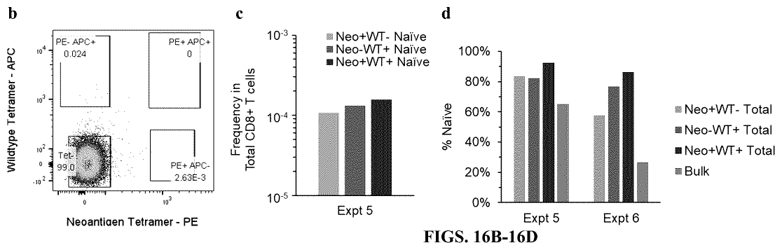

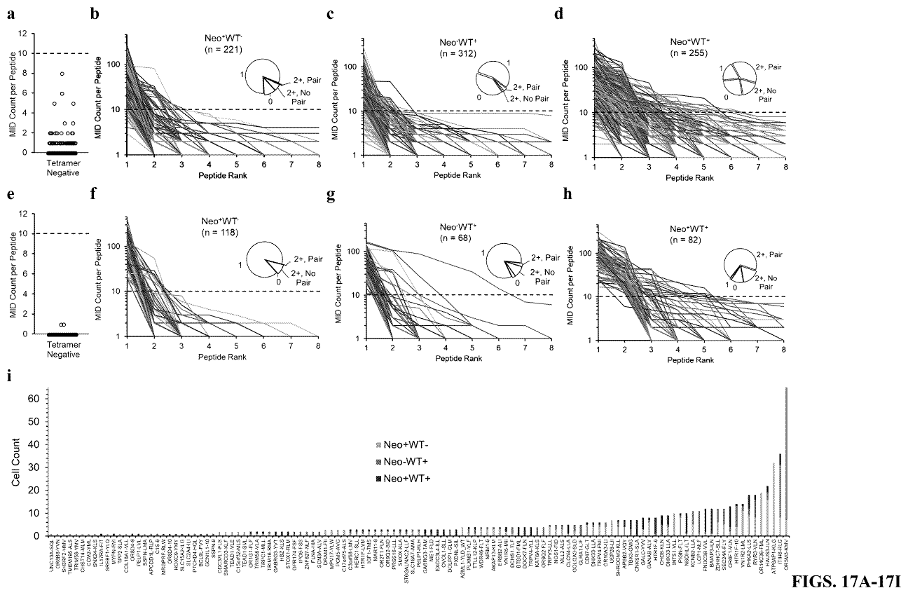

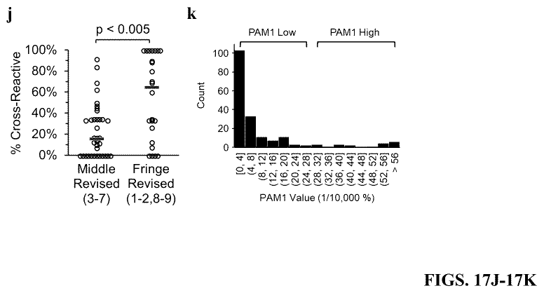

Five peptides in Experiment 3 and 4 had no detected T cell binding. Further analysis showed no difference in the pMHC UV-exchange efficiency associated with detected and undetected peptides (FIG. 16). TetTCR-Seq on a subsequent donor using these 5 peptides showed that these antigen-binding T cells are present at low frequencies in blood. Furthermore, monoclonal T cell lines specific for 3 of the peptides were successfully generated and found that IVTT-generated pMHC tetramers stained similarly as their synthetic peptide counterparts. These results confirm that “undetected” peptide-binding T cells in Experiment 3 and 4 were more likely caused by low cell frequency rather than inefficient pMHC generation by IVTT.

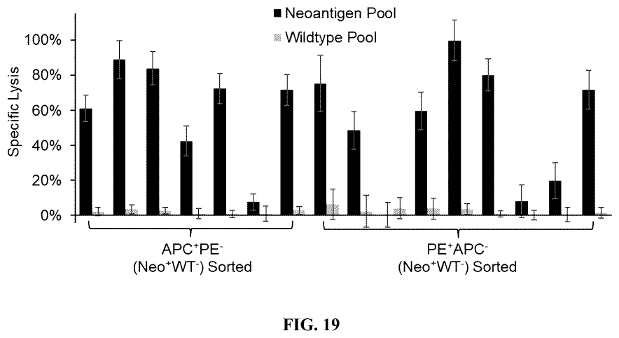

To test the feasibility of TetTCR-Seq to screen larger libraries, a 315 Neo-WT antigen pair library (1 WT is associated with 2 Neo) was assembled and T cell cross-reactivity was profiled across more than 1000 Tetramer⁺ CD8⁺ sorted single T cells from two donors, corresponding to Experiment 5 and 6 (FIGS. 2D and 17-18). Neo-antigens were selected with high predicted affinity for HLA-A2 from recent literature, and preference was given to those with positive binding and/or T cell assays. ELISA on all 315 pMHC species showed no difference in pMHC UV-exchange efficiency between detected and undetected peptides (FIG. 19).

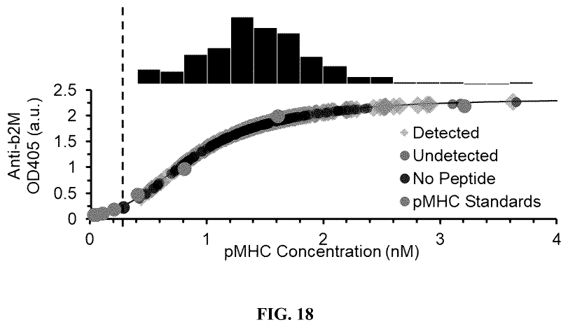

Similar to Experiment 3 and 4, neo-antigen mutations in the fringes had an elevated percentage of cross-reactive T cells than mutations in the middle (FIG. 2E-F). This difference increased when middle was extended to position 3-7 (FIG. 18J). This larger dataset also enabled us to examine the effect of neo-antigen mutation identity. The PAM1 matrix was used as a measure for chemical similarity between AAs. High PAM1 values correspond to a high mutational probability in evolution. It was found that neo-antigen mutations with high PAM1 values have a significantly higher percentage of cross-reactive T cells than those with low PAM1 values (FIG. 2F, 18K). Thus, in addition to mutation position, WT-binding T cells are more likely to recognize the neo-antigen if the mutated AA is chemically similar to the original. While these results show that mutation position and identity are two major factors that contribute to T cell cross-reactivity, large unaccounted variations still exist between peptides, highlighting the necessity for experimental screening against WT cross-reactivity when using neo-antigen based therapy in cancer.

Lastly, it was assessed the utility of TetTCR-Seq for isolating neo-antigen-specific TCRs with no cross-reactivity to WT. To this end, cell lines were generated from the Neo⁺WT⁻, Neo⁻WT⁺, and Neo⁺WT⁺ populations using the 40 Neo-WT pMHC tetramer library from Experiment 3 and 4. Each T cell line consist of 5 Tetramer⁺ cells sorted from the same population. These cell lines responded to Neo and WT antigens in a manner that matched their population gating scheme during sorting (FIG. 2G). The choice of fluorophore did not affect this functional profile, as tested by swapping the fluorophore encoding of the DNA-BC pMHC library (FIG. 20). The T cell lines were further characterized in Neo⁺WT⁻ and Neo⁺WT⁺ categories by TetTCR-Seq and found unique TCRs in each cell line targeting a wide range of antigens (FIG. 21A-B). Neo⁺WT⁺ cell lines identified as monoclonal were functional against the Neo-WT antigen pair identified by TetTCR-Seq, but not the other 19 Neo-WT pairs (FIG. 21C).

To directly show that TCR sequences isolated from primary T cells match the antigen specificity detected by the TetTCR-Seq, five TCRs were transduced from Experiment 3 and 4 into the TCR-deficient Jurkat 76 cell line. TCR-transduced Jurkat cells were stained with pMHC tetramers that corresponded to the neoantigen-WT paired specificity of the primary T cell (FIG. 2H, 22). Together, the TCR-transduced Jurkat and T cell line experiments show that TetTCR-Seq is not only capable of identifying cross-reactive TCRs on a large scale but can also identify mono-specific TCRs that are functionally reactive to Neo- but not WT-peptide in a high-throughput manner. Such TCRs could be therapeutically valuable in TCR re-directed adoptive cell transfer therapy.

In conclusion, it was shown that TetTCR-Seq can accurately link TCR sequences with multiple antigenic pMHC binders. This platform is general and can be broadly applied to interrogate antigen-binding T cells in clonally expanded or precursor T cell populations, from infection to autoimmune disease to cancer immunotherapy. With promising methods emerging for predicting antigenic pMHCs for groups of TCR sequences, TetTCR-Seq can not only expedite the discovery in this area but also help to experimentally validate informatically predicted antigens. The unique DNA-BC/IVTT approach enables the affordable and rapid generation of a large set of DNA-BC pMHC tetramers, making it possible to widely adopt TetTCR-Seq to accelerate T cell based scientific and clinical discoveries. Lastly, the pairing of TetTCR-Seq with recent advances in single-cell transcriptome and protein quantification signals a future in which integrated single T cell phenotype, TCR sequence, and pMHC-binding landscape can be measured at scale.

TABLE 2
Summary of the 6 main TetTCR-Seq experiments performed and blood donor characteristics. The
percentage difference between “DNA-BC” column and “Antigen Detection” column are those T cells without identified binding antigen
based on the criteria listed. These T cells correspond to grey lines in all the peptide rank curves.

| | | | | CMV | pMHC | Sorted | Cells |
|---|---|---|---|---|---|---|---|
| Expt | Expt Type | Age | Gender | Status | Library^{a} | Population | Sorted |
| 1 | 96 Foreign | 30 | Male | + | 29 Foreign (APC) | Foreign Naïve | 56 |
| | Endogenous | | | | 61 Endogenous (PE) | Foreign Non- | 32 |
| | | | | | 5 HCV-KLV + | Naïve | |
| | | | | | Mut. (APC) | Endogenous | 56 |
| | | | | | 1 Neg. Ctrl | Naïve | |
| | | | | | (PE, APC)^{e} | Endogenous | 23 |
| | | | | | | Non-Naïve | |
| | | | | | | HCV-KLV | 8 |
| | | | | | | Specific Clone | |
| | | | | | | Tetramer⁻ | 8 |
| 2 | 96 Foreign | 51 | Male | + | 29 Foreign (APC) | Foreign Naïve | 96 |
| | Endogenous | | | | 61 Endogenous (PE) | Foreign Non- | 88 |
| | | | | | 6 HCV-KLV + | Naïve | |
| | | | | | Mut. (APC)^{f} | Endogenous | 96 |
| | | | | | | Naïve | |
| | | | | | | Endogenous | 88 |
| | | | | | | Non-Naïve | |
| | | | | | | HCV-KLV | 8 |
| | | | | | | Specific Clone | |
| | | | | | | Tetramer⁻ | 8 |
| 3^{g} | 40 | 56 | Male | − | 20 Neoantigen (APC) | Neo⁺WT⁻ | 142 |
| | Neoantigen | 65 | Male | − | 20 Wildtype (PE) | Neo⁻WT⁺ | 43 |
| | Wildtype | | | | 1 HCV-KLV (PE, APC) | Neo⁺WT⁺ | 76 |
| | | | | | 1 Neg. Ctrl (PE, APC)^{e} | HCV-KLV | 12 |
| | | | | | | Specific Clone | |
| | | | | | | Tetramer⁻ | 12 |
| 4^{g} | 40 | 50 | Male | − | 20 Neoantigen (APC) | Neo⁺WT⁻ | 144 |
| | Neoantigen | 56 | Female | − | 20 Wildtype (PE) | Neo⁻WT⁺ | 44 |
| | Wildtype | | | | 4 MAGE-A (PE, APC)^{h} | Neo⁺WT⁺ | 108 |
| | | | | | | Tetramer⁻ | 35 |
| 5 | 315 | 47 | Female | − | 158 Neoantigen (PE)ⁱ | Neo⁺WT⁻ | 221 |
| | Neoantigen | | | | 157 Wildtype (APC) | Neo⁻WT⁺ | 312 |
| | Wildtype | | | | 1 HCV-KLV (PE, APC) | Neo⁺WT⁺ | 255 |
| | | | | | 1 Neg. Ctrl (PE, APC)^{e} | HCV-KLV | 8 |
| | | | | | | Specific Clone | |
| | | | | | | Tetramer⁻ | 8 |
| 6 | 315 | 58 | Male | − | 158 Neoantigen (PE)ⁱ | Neo⁺WT⁻ | 118 |
| | Neoantigen | | | | 157 Wildtype (APC) | Neo⁻WT⁺ | 68 |
| | Wildtype | | | | 1 HCV-KLV (PE, APC) | Neo⁺WT⁺ | 82 |
| | | | | | 1 Neg. Ctrl (PE, APC)^{e} | Tetramer⁻ | 6 |

Summary of the 6 main TetTCR-Seq experiments performed and blood donor characteristics. The
percentage difference between “DNA-BC” column and “Antigen Detection” column are those T cells without identified binding antigen
based on the criteria listed. These T cells correspond to grey lines in all the peptide rank curves.

| | Amplification Efficiency | | | | Antigen | Relevant |
|---|---|---|---|---|---|---|
| Expt | TCRα^{b} | TCRβ^{b} | TCRαβ^{b} | DNA-BC^{c} | Detection^{d} | Figures |
| 1 | 28 (50%) | 36 (64%) | 20 (36%) | 56 (100%) | 50 (89%) | Main Figure: |
| | 13 (41%) | 19 (59%) | 10 (31%) | 32 (100%) | 32 (100%) | 1b, 1d, 1e, 1g, 1h, 1i |
| | 37 (66%) | 45 (80%) | 34 (61%) | 56 (100%) | 55 (98%) | Supplementary: |
| | 9 (39%) | 12 (52%) | 4 (17%) | 23 (100%) | 23 (100%) | 6, 7, 8 |
| | 8 (100%) | 8 (100%) | 8 (100%) | 8 (100%) | 8 (100%) | |
| | n/a | n/a | n/a | 5 (63%) | 0 (0%) | |
| 2 | 74 (77%) | 78 (81%) | 59 (61%) | 96 (100%) | 85 (79%) | Supplementary: |
| | 67 (76%) | 62 (70%) | 54 (61%) | 88 (100%) | 84 (95%) | 6, 9 |
| | 75 (78%) | 81 (84%) | 64 (67%) | 96 (100%) | 92 (96%) | |
| | 79 (90%) | 83 (94%) | 77 (88%) | 87 (99%) | 75 (85%) | |
| | 7 (88%) | 7 (88%) | 7 (88%) | 8 (100%) | 7 (88%) | |
| | n/a | n/a | n/a | 7 (88%) | 0 (0%) | |
| 3^{g} | 112 (79%) | 130 (92%) | 106 (75%) | 142 (100%) | 127 (89%) | Main Figure: |
| | 36 (84%) | 34 (79%) | 30 (70%) | 43 (100%) | 43 (100%) | 2a-c |
| | 61 (80%) | 71 (93%) | 59 (78%) | 76 (100%) | 71 (93%) | Supplementary: |
| | 12 (100%) | 12 (100%) | 12 (100%) | 12 (100%) | 12 (100%) | 10-12, 14 |
| | n/a | n/a | n/a | 10 (83%) | 0 (0%) | |
| 4^{g} | 34 (24%) | 33 (23%) | 12 (8%) | 144 (100%) | 144 (100%) | Supplementary: |
| | 16 (36%) | 11 (25%) | 6 (14%) | 44 (100%) | 44 (100%) | 10, 13, 14 |
| | 30 (28%) | 31 (29%) | 11 (10%) | 108 (100%) | 95 (88%) | |
| | n/a | n/a | n/a | 13 (37%) | 0 (0%) | |
| 5 | 136 (62%) | 137 (62%) | 112 (51%) | 215 (97%) | 197 (89%) | Main Figure: |
| | 172 (55%) | 183 (59%) | 134 (43%) | 301 (96%) | 186 (60%) | 2d-f |
| | 140 (55%) | 150 (59%) | 108 (42%) | 249 (98%) | 189 (74%) | Supplementary: |
| | 6 (75%) | 6 (75%) | 6 (75%) | 7 (88%) | 7 (88%) | 15-17 |
| | n/a | n/a | n/a | 7 (88%) | 0 (0%) | |
| 6 | 97 (82%) | 99 (84%) | 86 (73%) | 118 (100%) | 118 (100%) | |
| | 53 (78%) | 58 (85%) | 46 (68%) | 68 (100%) | 66 (97%) | |
| | 62 (76%) | 67 (82%) | 52 (63%) | 82 (100%) | 72 (88%) | |
| | n/a | n/a | n/a | 1 (17%) | 0 (0%) | |

^{a}Detailed summary in Supplementary Table. Shown is the number of peptides, peptide category, and fluorescent encoding.
^{b}Includes only cells containing productive TCRα and/or TCRβ sequences are included
^{c}Includes only cells with at least 100 reads of DNA-BC and this applies to Tetramer⁻ cells as well.
^{d}Includes only cells with at least one detected antigen from the MID threshold criteria
^{e}A DNA-BC pMHC tetramer UV-exchanged with a non HLA-A2 binding peptide, RLFAFVRFT
^{f}The library is the same as Expt 1, except for the replacement of the negative control peptide with an additional HCV-KLV mutant peptide, HCV-A9N. This peptide did not bind to the HCV-KLV Specific clone in a separate tetramer staining, and serves as a negative control.
^{g}Blood samples from two donors were pooled together in Experiment 3 and 4
^{h}The library is the same as Expt 3, except for the replacement of the negative control and HCV-KLV peptide with 4 peptides from the MAGE-A antigen family. 3 MAGE-A specific T cells were detected out of 298 cells and were not used for subsequent analysis.
ⁱNeo-antigen/WT pairs are used for all antigens except for DHX33-LLA, which have two neo-antigens with substitutions K5T and M4I. One T cell was found to be cross-reactive to all three peptides.

TABLE 3
TetTCR-Seq summary for experiment 1

| Cell | Sorted | Detected Peptide by MID Count | | | | | TCRα,1 | | TCRα,2 | | TCRβ | | SEQ ID NOs |
|---|---|---|---|---|---|---|---|---|---|---|---|---|---|
| Name | Population | Rank 1 | Rank 2 | Rank 3 | Rank 4 | Rank 5 | TRAV | CDR3α | TRAV | CDR3a | TRBV | CDR3β | (L to R) |
| AA1 | Naïve | ZNT8- | 0 | 0 | 0 | 0 | | | | | 6-2*01,6- | CASSYSENEQFF | 1628 |
| | Endogenous | LLS | | | | | | | | | 3*01 | | |
| AA10 | Naïve | MART1- | 0 | 0 | 0 | 0 | 12-2*01 | CGGQAGTALIF | | | 6-1*01 | CASRSYVASSNE | 1549 |
| | Endogenous | A2L | | | | | | | | | | QFF | 1629 |
| AA11 | Naïve | MART1- | 0 | 0 | 0 | 0 | 12-2*01 | CAVNGGNQFY | | | 28*01 | CASTQWYGGGT | 1550 |
| | Endogenous | A2L | | | | | | F | | | | PPYF | 1630 |
| AA12 | Naïve | MART1- | 0 | 0 | 0 | 0 | 12-2*01 | CAVGRDDKIIF | | | 7-2*01 | CASSLTTGVFSQ | 1551 |
| | Endogenous | A2L | | | | | | | | | | PQHF | 1631 |
| AA2 | Naïve | MART1- | 0 | 0 | 0 | 0 | 17*01 | CATCMDSNYQL | | | 15*01 | CATSPYSVTTFA | 1552 |
| | Endogenous | A2L | | | | | | IW | | | | NTIYF | 1632 |
| AA3 | Naïve | MAGEA10- | 0 | 0 | 0 | 0 | | | | | 2*01 | CAGMTVTEAFF | 1633 |
| | Endogenous | GLY | | | | | | | | | | | |
| AA4 | Naïve | MART1- | 0 | 0 | 0 | 0 | | | | | 4-2*01 | CASSQALLAPSTD | 1634 |
| | Endogenous | A2L | | | | | | | | | | TQYF | |
| AA5 | Naïve | MART1- | 0 | 0 | 0 | 0 | 12-2*01 | CAVTTDSWGKL | | | 2*01 | CASSEGGIGELF | 1553 |
| | Endogenous | A2L | | | | | | QF | | | | F | 1635 |
| AA6 | Naïve | MART1- | 0 | 0 | 0 | 0 | | | | | | | |
| | Endogenous | A2L | | | | | | | | | | | |
| AA7 | Naïve | MART1- | 0 | 0 | 0 | 0 | | | | | 4-1*01 | CASSQDTDGRM | 1636 |
| | Endogenous | A2L | | | | | | | | | | FF | |
| AA8 | Naïve | MART1- | 0 | 0 | 0 | 0 | 12-2*01 | CAVNPGGADG | | | 6-1*01 | CASSEAPGTSV | 1554 |
| | Endogenous | A2L | | | | | | LTF | | | | GGLFF | 1637 |
| AA9 | Naïve | MART1- | 0 | 0 | 0 | 0 | 12-2*01 | CAVSGSARQLT | | | 28*01 | CASTTGDGLGAF | 1555 |
| | Endogenous | A2L | | | | | | F | | | | F | 1638 |
| AB1 | Naïve | PPI-RLL | 0 | 0 | 0 | 0 | 8-1*01 | CAVNPRDNYG | | | 4-2*01 | CASSQDIGNFEQ | 1556 |
| | Endogenous | | | | | | | QNFVF | | | | FF | 1639 |
| AB11 | Naïve | MART1- | 0 | 0 | 0 | 0 | | | | | | | |
| | Endogenous | A2L | | | | | | | | | | | |
| AB12 | Naïve | ZNT8- | 0 | 0 | 0 | 0 | 12-3*01 | CAAGGSYIPTF | | | 28*01 | CASSGTGGYSG | 1557 |
| | Endogenous | LLS | | | | | | | | | | ANVLTF | 1640 |
| AB3 | Naïve | MART1- | 0 | 0 | 0 | 0 | | | | | | | |
| | Endogenous | A2L | | | | | | | | | | | |
| AB4 | Naïve | MART1- | 0 | 0 | 0 | 0 | 12-2*01 | CAVNTGFQKLV | | | 27*01 | CASSEANEKLFF | 1558 |
| | Endogenous | A2L | | | | | | F | | | | | 1641 |
| AB5 | Naïve | MART1- | 0 | 0 | 0 | 0 | 12-2*01 | CAVNGNNRLAF | | | 4-1*01 | CASSQAPLASG | 1559 |
| | Endogenous | A2L | | | | | | | | | | GYTF | 1642 |
| AB6 | Naïve | MART1- | 0 | 0 | 0 | 0 | 12-2*01 | CAVQGGGSQG | | | 7-2*01 | CASSLAGQVFS | 1560 |
| | Endogenous | A2L | | | | | | NLIF | | | | GELFF | 1643 |
| AB7 | Naïve | MART1- | 0 | 0 | 0 | 0 | 12-2*01 | CAAGGSQGNLI | | | 4-2*01 | CASSQGTINTGE | 1561 |
| | Endogenous | A2L | | | | | | F | | | | LFF | 1644 |
| AB8 | Naïve | MART1- | 0 | 0 | 0 | 0 | 12-2*01 | CAVNIPTF | | | 20-1*01 | CSARDGTSSGY | 1562 |
| | Endogenous | A2L | | | | | | | | | | F | 1645 |
| AB9 | Naïve | MART1- | 0 | 0 | 0 | 0 | | | | | 19*01 | CASMPRGFPSD | 1646 |
| | Endogenous | A2L | | | | | | | | | | EQFF | |
| AC1 | Naïve | MART1- | 0 | 0 | 0 | 0 | | | | | 4-2*01 | CASSQDWVAEQ | 1647 |
| | Endogenous | A2L | | | | | | | | | | YF | |
| AC10 | Naïve | MART1- | 0 | 0 | 0 | 0 | 12-2*01 | CAVSGTASKLT | | | 6-6*01 | CASSYGTGDGY | 1563 |
| | Endogenous | A2L | | | | | | F | | | | TF | 1648 |
| AC11 | Naïve | GP100- | 0 | 0 | 0 | 0 | 13-2*01 | CAEKGGGGAD | | | | | 1564 |
| | Endogenous | IMD | | | | | | GLTF | | | | | |
| AC12 | Naïve | MART1- | 0 | 0 | 0 | 0 | 23/DV6* | CAASKEAAGNK | 12-2*01 | CAVKDGQNF | 28*01 | CASSLGLGQPQ | 1565162 |
| | Endogenous | A2L | | | | | 01 | LTF | | VF | | GF | 51649 |
| AC2 | Naïve | GP100- | GP100- | 0 | 0 | 0 | 26-1*01 | CIVRGFAYGQN | 16*01 | CALSPGYNF | 18*01 | CASSSRDRSSST | 1566162 |
| | Endogenous | IMD | ITD | | | | | FVF | | NKFYF | | EAFF | 61650 |
| AC3 | Naïve | MART1- | 0 | 0 | 0 | 0 | | | | | | | |
| | Endogenous | A2L | | | | | | | | | | | |
| AC4 | Naïve | MART1- | 0 | 0 | 0 | 0 | 12-2*01 | CAVSDGQKLLF | | | 14*01 | CASSQAGVGGE | 1567 |
| | Endogenous | A2L | | | | | | | | | | LFF | 1651 |
| AC5 | Naïve | MART1- | 0 | 0 | 0 | 0 | | | | | 28*01 | CASSLPGLASHE | 1652 |
| | Endogenous | A2L | | | | | | | | | | QFF | |
| AC6 | Naïve | MART1- | 0 | 0 | 0 | 0 | 12-2*01 | CAVTRGGADGL | | | 6-5*01 | CASSYSGLGQP | 1568 |
| | Endogenous | A2L | | | | | | TF | | | | QHF | 1653 |
| AC7 | Naïve | MART1- | 0 | 0 | 0 | 0 | 13-2*01 | CAENRDGDDKII | | | | | 1569 |
| | Endogenous | A2L | | | | | | F | | | | | |
| AC8 | Naïve | MART1- | 0 | 0 | 0 | 0 | 12-2*01 | CAASGGGADG | | | 28*01 | CASSSTVYNEQF | 1570 |
| | Endogenous | A2L | | | | | | LTF | | | | G | 1654 |
| AC9 | Naïve | MART1- | 0 | 0 | 0 | 0 | 12-2*01 | CAVRTQIIF | | | 27*01 | CASSRSPGGVY | 1571 |
| | Endogenous | A2L | | | | | | | | | | EQYF | 1655 |
| AD1 | Naïve | MART1- | 0 | 0 | 0 | 0 | | | | | | | |
| | Endogenous | A2L | | | | | | | | | | | |
| AD10 | Naïve | MART1- | 0 | 0 | 0 | 0 | 41*01 | CAVRSERSGG | | | 27*01 | CASSPSPAGAYE | 1572 |
| | Endogenous | A2L | | | | | | GADGLTF | | | | QYF | 1656 |
| AD11 | Naïve | CD1-LLG | 0 | 0 | 0 | 0 | 12-2*01 | CAVNDYKLSF | | | 27*01 | CASSWTGANYG | 1573 |
| | Endogenous | | | | | | | | | | | YTF | 1657 |
| AD12 | Naïve | MART1- | 0 | 0 | 0 | 0 | 12-2*01 | CAVNTGFQKLV | | | 27*01 | CASSPNLAGEE | 1558 |
| | Endogenous | A2L | | | | | | F | | | | QYF | 1658 |
| AD2 | Naïve | MART1- | 0 | 0 | 0 | 0 | | | | | | | |
| | Endogenous | A2L | | | | | | | | | | | |
| AD3 | Naïve | MART1- | 0 | 0 | 0 | 0 | 12-2*01 | CAAEFYF | | | 11-1*01 | CASSLGQGQPQ | 1574 |
| | Endogenous | A2L | | | | | | | | | | HF | 1659 |
| AD4 | Naïve | MART1- | 0 | 0 | 0 | 0 | 12-2*01 | CASDNNARLMF | | | 4-1*01 | CASSQEVVANN | 1575 |
| | Endogenous | A2L | | | | | | | | | | EQFF | 1660 |
| AD5 | Naïve | MART1- | 0 | 0 | 0 | 0 | | | | | 27*01 | CASSLGGNTGEL | 1661 |
| | Endogenous | A2L | | | | | | | | | | FF | |
| AD6 | Naïve | MART1- | 0 | 0 | 0 | 0 | 12-2*01 | CAVIRSGGYNK | | | 5-6*01 | CASSLELAGGPA | 1576 |
| | Endogenous | A2L | | | | | | LIF | | | | FF | 1662 |
| AD7 | Naïve | MART1- | 0 | 0 | 0 | 0 | | | | | | | |
| | Endogenous | A2L | | | | | | | | | | | |
| AD8 | Naïve | MART1- | 0 | 0 | 0 | 0 | | | | | 2*01 | CASRAGIQSGEL | 1663 |
| | Endogenous | A2L | | | | | | | | | | FF | |
| AD9 | Naïve | MART1- | 0 | 0 | 0 | 0 | | | | | 27*01 | CASSPSGHYEQ | 1664 |
| | Endogenous | A2L | | | | | | | | | | YF | |
| AE1 | Naïve Foreign | CMV- | 0 | 0 | 0 | 0 | 12-2*01 | CAGFSGGYNKL | | | 9*01 | CASSRGTGGYE | 1577 |
| | | MLN | | | | | | IF | | | | QFF | 1665 |
| AE10 | Naïve Foreign | HTLV- | 0 | 0 | 0 | 0 | 12-3*01 | CTSRVSDGQKL | | | | | 1578 |
| | | LLF | | | | | | LF | | | | | |
| AE11 | Naïve Foreign | YFV-LLW | 0 | 0 | 0 | 0 | | | | | 12- | CASSLSGDEQYF | 1666 |
| | | | | | | | | | | | 3*01,12- | | |
| | | | | | | | | | | | 4*01 | | |
| AE12 | Naïve Foreign | YFV-LLW | 0 | 0 | 0 | 0 | 12-1*01 | CVVNNDKIIF | | | 27*01 | CASSLTPSASGY | 1579 |
| | | | | | | | | | | | | EQYF | 1667 |
| AE2 | Naïve Foreign | HSV-SLP | 0 | 0 | 0 | 0 | | | | | | | |
| AE3 | Naïve Foreign | HSV-SLP | 0 | 0 | 0 | 0 | | | | | | | |
| AE4 | Naïve Foreign | HSV-SLP | 0 | 0 | 0 | 0 | | | | | | | |
| AE5 | Naïve Foreign | YFV-LLW | 0 | 0 | 0 | 0 | | | | | | | |
| AE6 | Naïve Foreign | IVPA- | 0 | 0 | 0 | 0 | | | | | | | |
| | | FMY | | | | | | | | | | | |
| AE7 | Naïve Foreign | ALADH- | 0 | 0 | 0 | 0 | 12-1*01 | CVVNEYSSASK | | | 14*01 | CASSQGWDEQY | 1580 |
| | | VLM | | | | | | IF | | | | F | 1668 |
| AE8 | Naïve Foreign | ALADH- | 0 | 0 | 0 | 0 | | | | | 9*01 | CASSTLSGNYNE | 1669 |
| | | VLM | | | | | | | | | | QFF | |
| AE9 | Naïve Foreign | HCV-L21 | 0 | 0 | 0 | 0 | | | | | 28*01 | CASGSVPEQYF | 1670 |
| AF1 | Naïve Foreign | YFV-LLW | 0 | 0 | 0 | 0 | 12-1*01 | CVVAEARLMF | | | 27*01 | CASSPGTGGTY | 1581 |
| | | | | | | | | | | | | EQYF | 1671 |
| AF10 | Naïve Foreign | EBV-YLQ | 0 | 0 | 0 | 0 | | | | | 27*01 | CASSGLAGFSP | 1672 |
| | | | | | | | | | | | | QETQYF | |
| AF11 | Naïve Foreign | YFV-LLW | 0 | 0 | 0 | 0 | | | | | 9*01 | CASSGGTGAYE | 1673 |
| | | | | | | | | | | | | QYF | |
| AF12 | Naïve Foreign | HBV- | 0 | 0 | 0 | 0 | 12-2*01 | CAVNGANDYKL | | | | | 1582 |
| | | WLS | | | | | | SF | | | | | |
| AF2 | Naïve Foreign | HCV-LLF | 0 | 0 | 0 | 0 | | | | | 28*01 | CASSAGASIEQY | 1674 |
| | | | | | | | | | | | | F | |
| AF3 | Naïve Foreign | EBV-YLQ | 0 | 0 | 0 | 0 | | | | | | | |
| AF4 | Naïve Foreign | HBV- | 0 | 0 | 0 | 0 | | | | | 3-1*01 | CASSLGQGGVG | 1675 |
| | | WLS | | | | | | | | | | EKLFF | |
| AF5 | Naïve Foreign | HTLV- | 0 | 0 | 0 | 0 | 38- | CAYSMLDRLMF | | | 15*01 | CATRKSYNSPLH | 1583 |
| | | LLF | | | | | 2/DV8* | | | | | F | 1676 |
| | | | | | | | 01 | | | | | | |
| AF6 | Naïve Foreign | IVPA- | 0 | 0 | 0 | 0 | | | | | | | |
| | | FMY | | | | | | | | | | | |
| AF7 | Naïve Foreign | HTLV- | 0 | 0 | 0 | 0 | | | | | | | |
| | | LLF | | | | | | | | | | | |
| AF8 | Naïve Foreign | YFV-LLW | 0 | 0 | 0 | 0 | 8-3*01 | CAVGSDSSYKL | | | 4-1*01 | CASSQAQGTYE | 1584 |
| | | | | | | | | IF | | | | QYF | 1677 |
| AF9 | Naïve Foreign | HTLV- | 0 | 0 | 0 | 0 | | | | | | | |
| | | LLF | | | | | | | | | | | |
| AG1 | Naïve Foreign | CMV- | 0 | 0 | 0 | 0 | | | | | 27*01 | CASSLGWGYEQ | 1678 |
| | | MLN | | | | | | | | | | YF | |
| AG10 | Naïve Foreign | CMV- | 0 | 0 | 0 | 0 | 12-2*01 | CAVGIYNQGGK | | | 4-1*01 | CASSPGLDYEQ | 1585 |
| | | MLN | | | | | | LIF | | | | YF | 1679 |
| AG11 | Naïve Foreign | IV-GIL | GLNS- | 0 | 0 | 0 | | | | | | | |
| | | | GLL | | | | | | | | | | |
| AG12 | Naïve Foreign | YFV-LLW | 0 | 0 | 0 | 0 | | | | | 12- | CASTRQFNQPQ | 1680 |
| | | | | | | | | | | | 3*01,12- | HF | |
| | | | | | | | | | | | 4*01 | | |
| AG2 | Naïve Foreign | YFV-LLW | 0 | 0 | 0 | 0 | 8-1*01 | CAVRRDDKIIF | | | | | 1586 |
| AG3 | Naïve Foreign | YFV-LLW | 0 | 0 | 0 | 0 | 12-2*01 | CAVNEGTGNQ | | | 4-1*01 | CASSQGGGTEA | 1587 |
| | | | | | | | | FYF | | | | FF | 1681 |
| AG4 | Naïve Foreign | HPV-YML | 0 | 0 | 0 | 0 | | | | | | | |
| AG5 | Naïve Foreign | EBV-YVL | 0 | 0 | 0 | 0 | 12-2*01 | CAVKGGGADG | | | 29-1*01 | CSALTGSSYEQY | 1588 |
| | | | | | | | | LTF | | | | F | 1682 |
| AG7 | Naïve Foreign | YFV-LLW | 0 | 0 | 0 | 0 | 12-2*01 | CAEGGGADGL | | | 9*01 | CASSGGYEQYF | 1589 |
| | | | | | | | | TF | | | | | 1683 |
| AG8 | Naïve Foreign | YFV-LLW | 0 | 0 | 0 | 0 | 12-1*01 | CVVNMGKNGQ | | | 27*01 | CASSFGDSYEQ | 1590 |
| | | | | | | | | KLLF | | | | YF | 1684 |
| AG9 | Naïve Foreign | HTLV- | 0 | 0 | 0 | 0 | 29/DVS* | CAALISNFGNE | | | | | 1591 |
| | | LLF | | | | | 01 | KLTF | | | | | |
| AH1 | Naïve Foreign | IV-GIL | 0 | 0 | 0 | 0 | 27*01 | CAGGGSQGNLI | | | | | 1592 |
| | | | | | | | | F | | | | | |
| AH10 | Naïve Foreign | YFV-LLW | 0 | 0 | 0 | 0 | | | | | 9*01 | CASSLSGSSYEQ | 1685 |
| | | | | | | | | | | | | YF | |
| AH11 | Naïve Foreign | YFV-LLW | 0 | 0 | 0 | 0 | 38- | CASLGQGAQKL | | | 10-3*01 | CAISEASGVTYE | 1593 |
| | | | | | | | 2/DV8* | VF | | | | QYF | 1686 |
| | | | | | | | 01 | | | | | | |
| AH12 | Naïve Foreign | CMV- | 0 | 0 | 0 | 0 | | | | | 4-3*01 | CASSQGQGYEQ | 1687 |
| | | MLN | | | | | | | | | | YF | |
| AH2 | Naïve Foreign | CMV- | 0 | 0 | 0 | 0 | | | | | 25-1*01 | CASSGSRVPYE | 1688 |
| | | MLN | | | | | | | | | | QYF | |
| AH3 | Naïve Foreign | 0 | 0 | 0 | 0 | 0 | 12-2*01 | CAVNQAGTALI | | | 4-1*01 | CASSQTGTGAY | 1594 |
| | | | | | | | | F | | | | EQYF | 1689 |
| AH4 | Naïve Foreign | 0 | 0 | 0 | 0 | 0 | 5*01 | CAEYSSASKIIF | | | 20-1*01 | CSANRQGSIYF | 1595 |
| | | | | | | | | | | | | | 1690 |
| AH5 | Naïve Foreign | GLNS- | 0 | 0 | 0 | 0 | 12-2*01 | CAVNRDSGTYK | | | 27*01 | CASSFEWSYEQ | 1596 |
| | | GLL | | | | | | YIF | | | | YF | 1691 |
| AH6 | Naïve Foreign | HTLV- | 0 | 0 | 0 | 0 | 24*01 | CASISLDSNYQL | | | | | 1597 |
| | | LLF | | | | | | IW | | | | | |
| AH7 | Naïve Foreign | YFV-LLW | 0 | 0 | 0 | 0 | | | | | 12- | CASSHRGYEQY | 1692 |
| | | | | | | | | | | | 3*01,12- | F | |
| | | | | | | | | | | | 4*01 | | |
| AH8 | Naïve Foreign | CMV- | 0 | 0 | 0 | 0 | | | | | 28*01 | CASSPIDRAGGP | 1693 |
| | | MLN | | | | | | | | | | YEQYF | |
| AH9 | Naïve Foreign | HTLV- | 0 | 0 | 0 | 0 | | | | | | | |
| | | LLF | | | | | | | | | | | |
| BA1 | Naïve | MART1- | 0 | 0 | 0 | 0 | 12-2*01 | CAVGREAAGN | | | 6-4*01 | CASSLTSGSFAG | 1593 |
| | Endogenous | A2L | | | | | | KLTF | | | | ELFF | 1694 |
| BA10 | Naïve | MART1- | 0 | 0 | 0 | 0 | 16*01 | CALSRPSRGSQ | | | 28*01 | CASSPQGSGGE | 1599 |
| | Endogenous | A2L | | | | | | GNLIF | | | | AFF | 1695 |
| BA12 | Naïve Foreign | YFV-LLW | 0 | 0 | 0 | 0 | 26-1*01 | CIVAAISGSARQ | | | 6-2*01,6- | CASSYGGGYEQ | 1600 |
| | | | | | | | | LTF | | | 3*01 | YF | 1696 |
| BA2 | Naïve | MART1- | 0 | 0 | 0 | 0 | 12-2*01 | CAVSGGGADG | | | 28*01 | CASSALGINEQF | 1601 |
| | Endogenous | A2L | | | | | | LTF | | | | F | 1697 |
| BA3 | Naïve | MART1- | 0 | 0 | 0 | 0 | 12-2*01 | CAVNVQGGSE | | | 28*01 | CASSWTGGGQP | 1602 |
| | Endogenous | A2L | | | | | | KLVF | | | | QHF | 1698 |
| BA4 | Naïve | IGRP- | 0 | 0 | 0 | 0 | | | | | | | |
| | Endogenous | VLF | | | | | | | | | | | |
| BA5 | Naïve | MART1- | 0 | 0 | 0 | 0 | | | | | 6-5*01 | CASNQGPGNTIY | 1699 |
| | Endogenous | A2L | | | | | | | | | | F | |
| BA6 | Naïve | MART1- | 0 | 0 | 0 | 0 | 12-2*01 | CAVNKGFQKLV | | | 27*01 | CASSDSYEQYF | 1603 |
| | Endogenous | A2L | | | | | | F | | | | | 1700 |
| BA7 | Naïve | GP100- | GP100- | 0 | 0 | 0 | 17*01 | CATDGRGSTLG | | | | | 1604 |
| | Endogenous | IMD | ITD | | | | | RLYF | | | | | |
| BA8 | Naïve | PPI-15- | 0 | 0 | 0 | 0 | 12-3*01 | CAMSESDGQK | | | 4-1*01 | CASSLVPLSPEQ | 1605 |
| | Endogenous | 23 | | | | | | LLF | | | | YF | 1701 |
| BA9 | Naïve | MART1- | 0 | 0 | 0 | 0 | 13-1*01 | CAPPGDGNNR | | | 12- | CASSLGAGGGG | 1606 |
| | Endogenous | A2L | | | | | | LAF | | | 3*01,12- | TQYF | 1702 |
| | | | | | | | | | | | 4*01 | | |
| BB1 | Naïve Foreign | HBV- | 0 | 0 | 0 | 0 | | | | | 28*01 | CASSQQGVWGT | 1703 |
| | | WLS | | | | | | | | | | GELFF | |
| BB10 | Non-Naïve | IV-GIL | 0 | 0 | 0 | 0 | 27*01 | CAGAGGGSQG | | | 19*01 | CASSPRSTDTQYF | 1607 |
| | Foreign | | | | | | | NLIF | | | | F | 1704 |
| BB11 | Non-Naïve | CMV-NLV | 0 | 0 | 0 | 0 | | | | | 28*01 | CASSFQGYTEAFF | 1705 |
| | Foreign | | | | | | | | | | | F | |
| BB12 | Non-Naïve | CMV-NLV | 0 | 0 | 0 | 0 | | | | | | | |
| | Foreign | | | | | | | | | | | | |
| BB2 | Naïve Foreign | YFV-LLW | 0 | 0 | 0 | 0 | 12-2*01 | CAVNSDGQKLL | | | | | 1608 |
| | | | | | | | | F | | | | | |
| BB3 | Naïve Foreign | 0 | 0 | 0 | 0 | 0 | 3*01 | CAVRDMGSNY | | | 6-6*01 | CASSYAQGAET | 1609 |
| | | | | | | | | QLIW | | | | QYF | 1706 |
| BB4 | Naïve Foreign | HTLV- | 0 | 0 | 0 | 0 | 29/DVS* | CAASASTDKLIF | | | 27*01 | CASSLGLADPNN | 1610 |
| | | LLF | | | | | 01 | | | | | EQFF | 1707 |
| BB5 | Naïve Foreign | IV-GIL | 0 | 0 | 0 | 0 | 27*01 | CAGASTGGDS | | | | | 1611 |
| | | | | | | | | GNTGKLIF | | | | | |
| BB6 | Naïve Foreign | HTLV- | 0 | 0 | 0 | 0 | 12-3*01 | CAMSLSNFGNE | | | 20-1*01 | CSARDGGLAGE | 1612 |
| | | LLF | | | | | | KLTF | | | | QKVGDTQYF | 1708 |
| BB7 | Naïve Foreign | CMV- | 0 | 0 | 0 | 0 | 8-4*01 | CAVILQGAQKL | 8-4*01 | CAVSSITQGG | 20-1*01 | CSARGAGVPYE | 1613162 |
| | | MLN | | | | | | VF | | SEKLVF | | QYF | 71709 |
| BB8 | Naïve Foreign | CMV- | 0 | 0 | 0 | 0 | | | | | 9*01 | CASSVGVSGSF | 1710 |
| | | MLN | | | | | | | | | | YEQYF | |
| BB9 | Non-Naïve | CMV-NLV | 0 | 0 | 0 | 0 | | | | | | | |
| | Foreign | | | | | | | | | | | | |
| BC1 | Non-Naïve | IV-GIL | 0 | 0 | 0 | 0 | | | | | 19*01 | CASWDRGNEQF | 1711 |
| | Foreign | | | | | | | | | | | F | |
| BC10 | Non-Naïve | CMV-NLV | 0 | 0 | 0 | 0 | | | | | | | |
| | Foreign | | | | | | | | | | | | |
| BC11 | Non-Naïve | CMV-NLV | 0 | 0 | 0 | 0 | 3*01 | CADYYGQNFVF | | | 28*01 | CASSFQGYTEAF | 1614 |
| | Foreign | | | | | | | | | | | F | 1705 |
| BC12 | Non-Naïve | IV-GIL | 0 | 0 | 0 | 0 | 27*01 | CAGQTGNTGKL | | | | | 1615 |
| | Foreign | | | | | | | IF | | | | | |
| BC2 | Non-Naïve | CMV- | 0 | 0 | 0 | 0 | 35*01 | CAGPMKTSYDK | | | 12- | CASSSANYGYTF | 1616 |
| | Foreign | NLV | | | | | | VIF | | | 3*01,12- | | 1712 |
| | | | | | | | | | | | 4*01 | | |
| BC3 | Non-Naïve | CMV- | 0 | 0 | 0 | 0 | | | | | 12- | CASSSANYGYTF | 1712 |
| | Foreign | NLV | | | | | | | | | 3*01,12- | | |
| | | | | | | | | | | | 4*01 | | |
| BC4 | Non-Naïve | CMV-NLV | 0 | 0 | 0 | 0 | | | | | 28*01 | CASSFQGYTEAF | 1705 |
| | Foreign | | | | | | | | | | | F | |
| BC5 | Non-Naïve | CMV-NLV | 0 | 0 | 0 | 0 | 3*01 | CADYYGQNFVF | | | 28*01 | CASSFQGYTEAF | 1614 |
| | Foreign | | | | | | | | | | | F | 1705 |
| BC6 | Non-Naïve | CMV-NLV | 0 | 0 | 0 | 0 | | | | | | | |
| | Foreign | | | | | | | | | | | | |
| BC7 | Non-Naïve | IV-GIL | 0 | 0 | 0 | 0 | | | | | | | |
| | Foreign | | | | | | | | | | | | |
| BC8 | Non-Naïve | IV-GIL | 0 | 0 | 0 | 0 | | | | | 3-1*01 | CASSQFRGGRD | 1713 |
| | Foreign | | | | | | | | | | | GYTF | |
| BC9 | Non-Naïve | CMV-NLV | 0 | 0 | 0 | 0 | 3*01 | CADYYGQNFVF | | | 28*01 | CASSFQGYTEAF | 1614 |
| | Foreign | | | | | | | | | | | F | 1705 |
| BD1 | Non-Naïve | CMV- | 0 | 0 | 0 | 0 | 35*01 | CAGPMKTSYDK | | | 12- | CASSSANYGYTF | 1616 |
| | Foreign | NLV | | | | | | VIF | | | 3*01,12- | | 1712 |
| | | | | | | | | | | | 4*01 | | |
| BD10 | Non-Naïve | CMV- | 0 | 0 | 0 | 0 | | | | | 12- | CASSSANYGYTF | 1712 |
| | Foreign | NLV | | | | | | | | | 3*01,12- | | |
| | | | | | | | | | | | 4*01 | | |
| BD11 | Non-Naïve | IV-GIL | 0 | 0 | 0 | 0 | | | | | | | |
| | Foreign | | | | | | | | | | | | |
| BD12 | Non-Naïve | CMV- | 0 | 0 | 0 | 0 | 24*01 | CARNTGNQFYF | | | 6-5*01 | CASSYSTGTAYG | 1617 |
| | Foreign | NLV | | | | | | | | | | YTF | 1714 |
| BD2 | Non-Naïve | CMV- | 0 | 0 | 0 | 0 | 35*01 | CAGPMKTSYDK | | | 12- | CASSSANYGYTF | 1616 |
| | Foreign | NLV | | | | | | VIF | | | 3*01,12- | | 1712 |
| | | | | | | | | | | | 4*01 | | |
| BD3 | Non-Naïve | IV-GIL | 0 | 0 | 0 | 0 | 30*01 | CGTLRNNNARL | | | | | 1618 |
| | Foreign | | | | | | | MF | | | | | |
| BD4 | Non-Naïve | IV-GIL | 0 | 0 | 0 | 0 | | | | | | | |
| | Foreign | | | | | | | | | | | | |
| BD5 | Non-Naïve | IV-GIL | 0 | 0 | 0 | 0 | 27*01 | CAGGGSQGNLI | | | 19*01 | CASSIRSSYEQY | 1592 |
| | Foreign | | | | | | | F | | | | F | 1715 |
| BD6 | Non-Naïve | IV-GIL | 0 | 0 | 0 | 0 | | | | | 9*01 | CASSARDFAYE | 1716 |
| | Foreign | | | | | | | | | | | QYF | |
| BD7 | Non-Naïve | CMV- | 0 | 0 | 0 | 0 | | | | | 12- | CASSSANYGYTF | 1712 |
| | Foreign | NLV | | | | | | | | | 3*01,12- | | |
| | | | | | | | | | | | 4*01 | | |
| BD8 | Non-Naïve | IV-GIL | 0 | 0 | 0 | 0 | 30*01 | CGTLRNNNARL | | | 19*01 | CASWDRGNEQF | 1618 |
| | Foreign | | | | | | | MF | | | | F | 1711 |
| BD9 | Non-Naïve | IV-GIL | 0 | 0 | 0 | 0 | 27*01 | CAGDKGGGSQ | | | | | 1619 |
| | Foreign | | | | | | | GNLIF | | | | | |
| BE1 | Non-Naïve | IV-GIL | 0 | 0 | 0 | 0 | | | | | | | |
| | Foreign | | | | | | | | | | | | |
| BE10 | Non-Naïve | MART1- | 0 | 0 | 0 | 0 | 12-2*01 | CAVTGGGTSY | | | 27*01 | CASSFALSNEAF | 1620 |
| | Endogenous | A2L | | | | | | GKLTF | | | | F | 1717 |
| BE11 | Non-Naïve | PD5-KLS | 0 | 0 | 0 | 0 | | | | | 6-1*01 | CASDEKLFF | 1718 |
| | Endogenous | | | | | | | | | | | | |
| BE12 | Non-Naïve | MART1- | 0 | 0 | 0 | 0 | | | | | 27*01 | CASSFAGTTEAF | 1719 |
| | Endogenous | A2L | | | | | | | | | | F | |
| BE2 | Non-Naïve | IV-GIL | 0 | 0 | 0 | 0 | | | | | | | |
| | Foreign | | | | | | | | | | | | |
| BE3 | Non-Naïve | IV-GIL | 0 | 0 | 0 | 0 | | | | | | | |
| | Foreign | | | | | | | | | | | | |
| BE4 | Non-Naïve | CMV-NLV | 0 | 0 | 0 | 0 | | | | | 28*01 | CASSFQGYTEAF | 1705 |
| | Foreign | | | | | | | | | | | F | |
| BE6 | Non-Naïve | MART1- | 0 | 0 | 0 | 0 | | | | | | | |
| | Endogenous | A2L | | | | | | | | | | | |
| BE7 | Non-Naïve | MART1- | 0 | 0 | 0 | 0 | | | | | 27*01 | CASSFAGTTEAF | 1719 |
| | Endogenous | A2L | | | | | | | | | | F | |
| BE8 | Non-Naïve | MART1- | 0 | 0 | 0 | 0 | 12-2*01 | CAVTAGGTSYG | | | | | 1621 |
| | Endogenous | A2L | | | | | | KLTF | | | | | |
| BE9 | Non-Naïve | MART1- | 0 | 0 | 0 | 0 | 12-2*01 | CAVTAGGTSYG | | | | | 1621 |
| | Endogenous | A2L | | | | | | KLTF | | | | | |
| BF1 | Non-Naïve | MART1- | 0 | 0 | 0 | 0 | | | | | 27*01 | CASSFAGTTEAF | 1719 |
| | Endogenous | A2L | | | | | | | | | | F | |
| BF10 | Non-Naïve | MART1- | 0 | 0 | 0 | 0 | | | | | | | 1621162 |
| | Endogenous | A2L | | | | | | | | | | | 4 |
| BF11 | Non-Naïve | MART1- | 0 | 0 | 0 | 0 | 12-2*01 | CAVTAGGTSYG | 38-2/DV8* | CAYRSPPSS | | | 1621161 |
| | Endogenous | A2L | | | | | | KLTF | 01 | EKLVF | | | 8 |
| BF12 | Non-Naïve | MART1- | 0 | 0 | 0 | 0 | 12-2*01 | CAVTAGGTSYG | 30*01 | CGTLRNNNA | | | |
| | Endogenous | A2L | | | | | | KLTF | | RLMF | | | |
| BF2 | Non-Naïve | TYR- | 0 | 0 | 0 | 0 | | | | | 29-1*01 | CSVTGTGLFDEQ | 1720 |
| | Endogenous | YMD | | | | | | | | | | YF | |
| BF3 | Non-Naïve | MART1- | 0 | 0 | 0 | 0 | | | | | 27*01 | CASSFAGTTEAF | 1719 |
| | Endogenous | A2L | | | | | | | | | | F | |
| BF4 | Non-Naïve | MART1- | 0 | 0 | 0 | 0 | | | | | 27*01 | CASSFAGTTEAFF | 1719 |
| | Endogenous | A2L | | | | | | | | | | F | |
| BF5 | Non-Naïve | MART1- | 0 | 0 | 0 | 0 | 12-2*01 | CAVTAGGTSYG | | | | | 1621 |
| | Endogenous | A2L | | | | | | KLTF | | | | | |
| BF6 | Non-Naïve | PD5-KLS | 0 | 0 | 0 | 0 | | | | | | | |
| | Endogenous | | | | | | | | | | | | |
| BF7 | Non-Naïve | MART1- | 0 | 0 | 0 | 0 | | | | | 27*01 | CASSFAGTTEAF | 1719 |
| | Endogenous | A2L | | | | | | | | | | F | |
| BF8 | Non-Naïve | CD1-LLG | 0 | 0 | 0 | 0 | 12-2*01 | CAVYSGGYNKL | | | 27*01 | CASSFVNTGELF | 1622 |
| | Endogenous | | | | | | | IF | | | | F | 1721 |
| BF9 | Non-Naïve | MART1- | 0 | 0 | 0 | 0 | | | | | | | |
| | Endogenous | A2L | | | | | | | | | | | |
| BG1 | Non-Naïve | MART1- | 0 | 0 | 0 | 0 | | | | | | | |
| | Endogenous | A2L | | | | | | | | | | | |
| BG2 | Non-Naïve | MART1- | 0 | 0 | 0 | 0 | 12-2*01 | CAVTAGGTSYG | | | 27*01 | CASSFAGTTEAF | 1621 |
| | Endogenous | A2L | | | | | | KLTF | | | | F | 1719 |
| BG3 | Non-Naïve | MART1- | 0 | 0 | 0 | 0 | | | | | | | |
| | Endogenous | A2L | | | | | | | | | | | |
| BG4 | Non-Naïve | MART1- | 0 | 0 | 0 | 0 | 12-2*01 | CALPSGNTPLV | | | 6-1*01 | CASSDPGSGAY | 1623 |
| | Endogenous | A2L | | | | | | F | | | | EQYF | 1722 |
| BH10 | Spike-In | HCV-K1Y | HCV- | HCV- | HCV- | HCV- | 38- | CAYRSPPSSEK | | | 28*01 | CASSFLGTGLNE | 1624 |
| | | | L2I | K1Y17V | K1S | KLV | 2/DV8* | LVF | | | | QYF | 1723 |
| | | | | | | | 01 | | | | | | |
| BH2 | Spike-In | HCV-K1Y | HCV- | HCV- | HCV- | HCV- | 38- | CAYRSPPSSEK | | | 28*01 | CASSFLGTGLNE | 1624 |
| | | | L2I | K1Y17V | K1S | KLV | 2/DV8* | LVF | | | | QYF | 1723 |
| | | | | | | | 01 | | | | | | |
| BH3 | Spike-In | HCV-K1Y | HCV- | HCV- | HCV- | HCV- | 38- | CAYRSPPSSEK | | | 28*01 | CASSFLGTGLNE | 1624 |
| | | | L2I | K1Y17V | K1S | KLV | 2/DV8* | LVF | | | | QYF | 1723 |
| | | | | | | | 01 | | | | | | |
| BH4 | Spike-In | HCV-K1Y | HCV- | HCV- | HCV- | HCV- | 38- | CAYRSPPSSEK | | | 28*01 | CASSFLGTGLNE | 1624 |
| | | | L2I | K1S | K1Y17V | KLV | 2/DV8* | LVF | | | | QYF | 1723 |
| | | | | | | | 01 | | | | | | |
| BH5 | Spike-In | HCV-K1Y | HCV- | HCV- | HCV- | HCV- | 38- | CAYRSPPSSEK | | | 28*01 | CASSFLGTGLNE | 1624 |
| | | | L2I | K1Y17V | K1S | KLV | 2/DV8* | LVF | | | | QYF | 1723 |
| | | | | | | | 01 | | | | | | |
| BH6 | Spike-In | HCV-K1Y | HCV- | HCV- | HCV- | HCV- | 38- | CAYRSPPSSEK | | | 28*01 | CASSFLGTGLNE | 1624 |
| | | | L2I | K1S | K1Y17V | KLV | 2/DV8* | LVF | | | | QYF | 1723 |
| | | | | | | | 01 | | | | | | |
| BH7 | Spike-In | HCV-K1Y | HCV- | HCV- | HCV- | HCV- | 38- | CAYRSPPSSEK | | | 28*01 | CASSFLGTGLNE | 1624 |
| | | | L2I | K1Y17V | K1S | KLV | 2/DV8* | LVF | | | | QYF | 1723 |
| | | | | | | | 01 | | | | | | |
| BH8 | Spike-In | HCV-K1Y | HCV- | HCV- | HCV- | HCV- | 38- | CAYRSPPSSEK | | | 28*01 | CASSFLGTGLNE | 1624 |
| | | | L2I | K1Y17V | K1S | KLV | 2/DV8* | LVF | | | | QYF | 1723 |
| | | | | | | | 01 | | | | | | |

TABLE 4
TetTCR summary for experiment 2

| | | | | | | | | | | | | | SEQ ID |
|---|---|---|---|---|---|---|---|---|---|---|---|---|---|
| Cell | Sorted | Detected Peptide by MID Count | | | | | TCRα,1 | | TCRα,2 | | TCRβ | | NO |
| Name | Population | Rank 1 | Rank 2 | Rank 3 | Rank 4 | Rank 5 | TRAV | CDR3α | TRAV | CDR3α | TRBV | CDR3β | (L to R) |
| WA11 | Clone | HCV_K1Y | HCV_L2I | HCV_K1S | HCV_ | HCV_ | 38- | CAYRSPPSSEKL | | | 28 | CASSFLGTGLNE | 1721 |
| | | | | | K1Y17V | KLV | 2/DV8 | VF | | | | QYF | 1889 |
| WB11 | Clone | HCV_K1Y | HCV_L2I | HCV_K1S | HCV_ | HCV_ | 38- | CAYRSPPSSEKL | | | 28 | CASSFLGTGLNE | 1724 |
| | | | | | K1Y17V | KLV | 2/DV8 | VF | | | | QYF | 1889 |
| WC11 | Clone | HCV_K1Y | HCV_K1S | HCV_L2I | HCV_ | HCV_ | 38- | CAYRSPPSSEKL | | | 28 | CASSFLGTGLNE | 1724 |
| | | | | | K1Y17V | KLV | 2/DV8 | VF | | | | QYF | 1889 |
| WD11 | Clone | 0 | 0 | 0 | 0 | 0 | | | | | | | |
| WE11 | Clone | HCV_K1Y | HCV_L2I | HCV_K1S | HCV_ | HCV_ | 38- | CAYRSPPSSEKL | | | 28 | CASSFLGTGLNE | 1724 |
| | | | | | K1Y17V | KLV | 2/DV8 | VF | | | | QYF | 1889 |
| WF11 | Clone | HCV_K1Y | HCV_L2I | HCV_ | HCV_K1S | HCV_ | 38- | CAYRSPPSSEKL | | | 28 | CASSFLGTGLNE | 1724 |
| | | | | K1Y17V | | KLV | 2/DV8 | VF | | | | QYF | 1889 |
| WG11 | Clone | HCV_K1Y | HCV_L2I | HCV_K1S | HCV_ | HCV_ | 38- | CAYRSPPSSEKL | | | 28 | CASSFLGTGLNE | 1724 |
| | | | | | K1Y17V | KLV | 2/DV8 | VF | | | | QYF | 1889 |
| WH11 | Clone | HCV_K1Y | HCV_L2I | HCV_K1S | HCV_ | HCV_ | 38- | CAYRSPPSSEKL | | | 28 | CASSFLGTGLNE | 1724 |
| | | | | | K1Y17V | KLV | 2/DV8 | VF | | | | QYF | 1889 |
| TA1 | Foreign_ | YFV_LLW | 0 | 0 | 0 | 0 | 2-Dec | CAVNSDGQKLLF | | | 3-Oct | CAISEGAAYGYTF | 1725 |
| | Naive | | | | | | | | | | | | 1890 |
| TA2 | Foreign_ | YFV_LLW | 0 | 0 | 0 | 0 | 2-Dec | CAPGDDKIIF | | | 15 | CATSSSGAYEQY | 1726 |
| | Naive | | | | | | | | | | | F | 1891 |
| TA3 | Foreign_ | EBV_YVL | 0 | 0 | 0 | 0 | 17 | CASSGLSSGGSY | | | | | 1727 |
| | Naive | | | | | | | IPTF | | | | | |
| TB1 | Foreign_ | HCV_L2I | 0 | 0 | 0 | 0 | 38- | CAYRLGGSEKLV | | | 13 | CASSFPPAGTGE | 1728 |
| | Naive | | | | | | 2/DV8 | F | | | | LFF | 1892 |
| TB2 | Foreign_ | CMV_MLN | 0 | 0 | 0 | 0 | 14/DV4 | CAMRGGLYNFNK | | | 1-Apr | CASSPQGQGESG | 1729 |
| | Naive | | | | | | | FYF | | | | ANVLTF | 1893 |
| TB3 | Foreign_ | YFV_LLW | 0 | 0 | 0 | 0 | 2-Dec | CAVTDYKLSF | 5 | CAGRSYNTNAGK | 1-Jun | CASSEALYEQYF | 1730 |
| | Naive | | | | | | | | | STF | | | 1879 |
| | | | | | | | | | | | | | 1894 |
| TC1 | Foreign_ | YFV_LLW | 0 | 0 | 0 | 0 | 2-Dec | CALQDDKIIF | | | 1-Apr | CASSQGAAYEQY | 1731 |
| | Naive | | | | | | | | | | | F | 1895 |
| TC2 | Foreign_ | 0 | 0 | 0 | 0 | 0 | | | | | | CASSDGVSYGYT | 1896 |
| | Naive | | | | | | | | | | 2 | F | |
| TC3 | Foreign_ | CMV_MLN | 0 | 0 | 0 | 0 | 17 | CATGDLGNQFYF | | | 8-Jul | CASSLGFGYEQF | 1732 |
| | Naive | | | | | | | | | | | F | 1897 |
| TD1 | Foreign_ | YFV_LLW | 0 | 0 | 0 | 0 | | | | | | | |
| | Naive | | | | | | | | | | | | |
| TD2 | Foreign_ | YFV_LLW | HA_VLH | 0 | 0 | 0 | 2-Dec | CAVNSDGQKLLF | | | | | 1725 |
| | Naive | | | | | | | | | | | | |
| TD3 | Foreign_ | EBV_GLC | 0 | 0 | 0 | 0 | | | | | 20-1 | CSARSGVGNTIYF | 1898 |
| | Naive | | | | | | | | | | | | |
| TE1 | Foreign_ | 0 | 0 | 0 | 0 | 0 | 14/DV4 | CAMRELTSGTYK | | | 20-1 | CSPLGGQGVWD | 1733 |
| | Naive | | | | | | | YIF | | | | EQFF | 1899 |
| TE2 | Foreign_ | CMV_NLV | 0 | 0 | 0 | 0 | 24 | CARNTGNQFYF | | | | | 1734 |
| | Naive | | | | | | | | | | | | |
| TE3 | Foreign_ | 0 | 0 | 0 | 0 | 0 | 5 | CAEQGGSARQLT | 12-2 | CAVSTDKLIF | | | 1735 |
| | Naive | | | | | | | F | | | | | 1880 |
| TF1 | Foreign_ | HCV_LLF | 0 | 0 | 0 | 0 | | | | | 3-Oct | CAISEPGTGDTEA | 1900 |
| | Naive | | | | | | | | | | | FF | |
| TF2 | Foreign_ | IV_GIL | 0 | 0 | 0 | 0 | 17 | CATDAVSGTGGT | | | 19 | CASSIYGAGYTEA | 1736 |
| | Naive | | | | | | | SYGKLTF | | | | FF | 1901 |
| TF3 | Foreign_ | YFV_LLW | 0 | 0 | 0 | 0 | 1-Dec | CVVNDNDMRF | | | 27 | CASSFGASYGYT | 1737 |
| | Naive | | | | | | | | | | | F | 1902 |
| TG1 | Foreign_ | YFV_LLW | HAFP_F | 0 | 0 | 0 | 10 | CVVSPYSRVCTQ | 12-2 | CAVSNQGGKLIF | | | 1738 |
| | Naive | | MN | | | | | L | | | | | 1881 |
| TG2 | Foreign_ | YFV_LLW | 0 | 0 | 0 | 0 | 2-Dec | CAVSEDKLSF | | | | | 1739 |
| | Naive | | | | | | | | | | | | |
| TG3 | Foreign_ | YFV_LLW | 0 | 0 | 0 | 0 | 2-Dec | CAVSGGSYIPTF | | | | | 1740 |
| | Naive | | | | | | | | | | | | |
| TH1 | Foreign_ | HSV_SLP | 0 | 0 | 0 | 0 | 2-Dec | CAVGSARQLTF | | | 24-1 | CATSVGSGPLST | 1741 |
| | Naive | | | | | | | | | | | DTQYF | 1903 |
| TH2 | Foreign_ | 0 | 0 | 0 | 0 | 0 | 1-Dec | CVVTASNDMRF | | | 14 | CASSQETSPNYG | 1742 |
| | Naive | | | | | | | | | | | YTF | 1904 |
| TH3 | Foreign_ | HCV_YLL | 0 | 0 | 0 | 0 | 38- | CACADYGGSQG | | | | | 1743 |
| | Naive | | | | | | 2/DV8 | NLIF | | | | | |
| UA1 | Foreign_ | CMV_NLV | IV_GIL | 0 | 0 | 0 | 24 | CATNTGNQFYF | | | 1-Jun | CASSPTTRTRYY | 1744 |
| | Naive | | | | | | | | | | | GYTF | 1905 |
| UA2 | Foreign_ | YFV_LLW | 0 | 0 | 0 | 0 | 1-Dec | CAFEGGKLIF | | | 20-1 | CSAIGPRGTDTQY | 1745 |
| | Naive | | | | | | | | | | | F | 1906 |
| UA3 | Foreign_ | 0 | 0 | 0 | 0 | 0 | 2-Dec | CAVNNDYKLSF | | | 3-1 | CASSQEMASVQE | 1746 |
| | Naive | | | | | | | | | | | TQYF | 1907 |
| UB1 | Foreign_ | YFV_LLW | 0 | 0 | 0 | 0 | 2-Dec | CAVTGNQFYF | | | 11-2 | CASSLGGQGAYE | 1747 |
| | Naive | | | | | | | | | | | QYF | 1908 |
| UB2 | Foreign_ | YFV_LLW | 0 | 0 | 0 | 0 | 2-Dec | CAGNNARLMF | | | 15 | CATSPRGGHEQY | 1748 |
| | Naive | | | | | | | | | | | F | 1909 |
| UB3 | Foreign_ | HCV_LLF | 0 | 0 | 0 | 0 | 38-1 | CALDAGNMLTF | | | 28 | CASLGLEYEQYF | 1749 |
| | Naive | | | | | | | | | | | | 1910 |
| UC1 | Foreign_ | YFV_LLW | 0 | 0 | 0 | 0 | 2-Dec | CAAGDARLMF | | | | | 1750 |
| | Naive | | | | | | | | | | | | |
| UC2 | Foreign_ | IVPA_FMY | 0 | 0 | 0 | 0 | 38- | CAYIWGDKIIF | | | | | 1753 |
| | Naive | | | | | | 2/DV8 | | | | | | 1911 |
| UC3 | Foreign_ | YFV_LLW | 0 | 0 | 0 | 0 | 39 | CAVDSGDMRF | | | | | 1752 |
| | Naive | | | | | | | | | | | | |
| UD1 | Foreign_ | YFV_LLW | 0 | 0 | 0 | 0 | | | | | | | |
| | Naive | | | | | | | | | | | | |
| UD2 | Foreign_ | 0 | 0 | 0 | 0 | 0 | 38- | CAYYGGSQGNLI | | | 13 | CASSATGVSPYE | 1753 |
| | Naive | | | | | | 2/DV8 | F | | | | QYF | 1911 |
| UD3 | Foreign_ | CMV_MLN | 0 | 0 | 0 | 0 | | | | | 19 | CASSQGLSYEQY | |
| | Naive | | | | | | | | | | | F | |
| UE1 | Foreign_ | 0 | 0 | 0 | 0 | 0 | 2-Dec | CAVITGGGNKLTF | | | 9 | CASSVAGSTEAFF | 1754 |
| | Naive | | | | | | | | | | | | 1913 |
| UE2 | Foreign_ | CMV_MLN | 0 | 0 | 0 | 0 | 3-Aug | CAVGMDSSYKLI | 10 | CVVSAMGGGNK | 1-Jun | CASNQPQHF | 1755 |
| | Naive | | | | | | | F | | LTF | | | 1882 |
| | | | | | | | | | | | | | 1914 |
| UE3 | Foreign_ | CMV_MLN | 0 | 0 | 0 | 0 | 4 | CLVGDVQEGFQK | | | 20-1 | CSARDPSQGGYE | |
| | Naive | | | | | | | LVF | | | | QYF | |
| UF1 | Foreign_ | YFV_LLW | IV_AIM | 0 | 0 | 0 | 1-Dec | CVVADDKIIF | | | 9 | CASSVDGGSQPQ | 1757 |
| | Naive | | | | | | | | | | | HF | 1916 |
| UF2 | Foreign_ | HSV_SLP | 0 | 0 | 0 | 0 | | | | | 2-Nov | CASSLPAGVGDT | 1917 |
| | Naive | | | | | | | | | | | QYF | |
| UF3 | Foreign_ | HCV_L2I | HCV_KLV | 0 | 0 | 0 | 38- | CASLRNMLTF | 38- | CALLDGNKLVF | 19 | CASSIGLNQPQHF | 1758 |
| | Naive | | | | | | 2/DV8 | | 2/DV8 | | | | 1883 |
| | | | | | | | | | | | | | 1918 |
| UG1 | Foreign_ | YFV_LLW | 0 | 0 | 0 | 0 | 24 | CARNTGNQFYF | | | 9 | CASSVGGVPYNE | 1734 |
| | Naive | | | | | | | | | | | QFF | 1919 |
| UG2 | Foreign_ | CMV_MLN | 0 | 0 | 0 | 0 | 2-Aug | CVVSVSGGYNKL | | | 2-Nov | CASSLVESEQFF | 1759 |
| | Naive | | | | | | | IF | | | | | 1920 |
| UG3 | Foreign_ | 0 | 0 | 0 | 0 | 0 | 14/DV4 | CAMRVRTWGQN | | | 12-3, | CASSFANSPLHF | 1760 |
| | Naive | | | | | | | FVF | | | 12-4 | | 1921 |
| UH1 | Foreign_ | YFV_LLW | 0 | 0 | 0 | 0 | 1-Dec | CVVSDDKIIF | | | 27 | CASSLTALGAAYV | 1761 |
| | Naive | | | | | | | | | | | YTF | 1922 |
| UH2 | Foreign_ | HCV_L2I | 0 | 0 | 0 | 0 | | | | | | | |
| | Naive | | | | | | | | | | 20-1 | CSATEGSGYTF | 1923 |
| UH3 | Foreign_ | YFV_LLW | 0 | 0 | 0 | 0 | | | | | 13 | CASSRRDSNTEA | 1924 |
| | Naive | | | | | | | | | | | FF | |
| VA1 | Foreign_ | YFV_LLW | 0 | 0 | 0 | 0 | 39 | CAWYSGGGADG | | | 5-May | CASSFWGADTQY | 1762 |
| | Naive | | | | | | | LTF | | | | F | 1925 |
| VA2 | Foreign_ | IV_GIL | 0 | 0 | 0 | 0 | 2-Dec | CAVSPFGNVLHC | | | 2 | CASTGQNPEAFF | 1763 |
| | Naive | | | | | | | | | | | | 1926 |
| VA3 | Foreign_ | HSV_SLP | 0 | 0 | 0 | 0 | 4-Aug | CAVSETGAGNNR | 41 | CAVEGSRLTF | 1-Jun | CASSEVRGPWAE | 1764 |
| | Naive | | | | | | | KLIW | | | | TQYF | 1863 |
| | | | | | | | | | | | | | 1967 |
| VB1 | Foreign_ | YFV_LLW | 0 | 0 | 0 | 0 | 2-Dec | CAVSDDKIIF | | | 15 | CATSRTGTGSTE | 1765 |
| | Naive | | | | | | | | | | | AFF | 1928 |
| VB2 | Foreign_ | 0 | 0 | 0 | 0 | 0 | | | | | | | |
| | Naive | | | | | | | | | | | | |
| VB3 | Foreign_ | IVPA_FMY | 0 | 0 | 0 | 0 | | | | | 3-Apr | CASSPTGTGYNE | 1929 |
| | Naive | | | | | | | | | | | QFF | |
| VC1 | Foreign_ | YFV_LLW | 0 | 0 | 0 | 0 | 2-Dec | CAVRLGGADGLT | | | 20-1 | CSAWWGAEQYF | 1766 |
| | Naive | | | | | | | F | | | | | 1930 |
| VC2 | Foreign_ | YFV_LLW | 0 | 0 | 0 | 0 | 14/DV4 | CAMRSSDPGGY | | | 19 | CASSIQGRGDTE | 1767 |
| | Naive | | | | | | | NKLIF | | | | AFF | 1931 |
| VC3 | Foreign_ | HAFP_GLS | HTLV_LLF | 0 | 0 | 0 | 1-Dec | CVVNGGGYQKV | | | 9 | CASSAGLFPEQFF | 1768 |
| | Naive | | | | | | | TF | | | | | 1932 |
| VD1 | Foreign_ | IV_GIL | 0 | 0 | 0 | 0 | 3-Dec | CAMSQDYNTDKL | | | 2 | CASRTRQEAFF | 1769 |
| | Naive | | | | | | | IF | | | | | 1933 |
| VD2 | Foreign_ | EBV_YVL | 0 | 0 | 0 | 0 | | | | | 19 | CASSIVGNTEAFF | 1934 |
| | Naive | | | | | | | | | | | | |
| VD3 | Foreign_ | ALADH_VLM | 0 | 0 | 0 | 0 | | | | | 8-Jul | CASSFWGLGELF | 1935 |
| | Naive | | | | | | | | | | | F | |
| VE1 | Foreign_ | YFV_LLW | 0 | 0 | 0 | 0 | 2-Dec | CAVTNDKIIF | | | 9 | CASSPMNEQFF | 1770 |
| | Naive | | | | | | | | | | | | 1936 |
| VE2 | Foreign_ | YFV_LLW | 0 | 0 | 0 | 0 | 27 | CAGASTGDYKLS | | | 25-1 | CASGRGPNYGYT | 1771 |
| | Naive | | | | | | | F | | | | F | 1937 |
| VE3 | Foreign_ | HPV_YML | 0 | 0 | 0 | 0 | | | | | 1-May | CASSLLGLIKETQ | 1938 |
| | Naive | | | | | | | | | | | YF | |
| VF1 | Foreign_ | YFV_LLW | 0 | 0 | 0 | 0 | | | | | 9 | CASSDSYEQYF | 1939 |
| | Naive | | | | | | | | | | | | |
| VF2 | Foreign_ | YFV_LLW | 0 | 0 | 0 | 0 | 2-Dec | CAVVGGYNKLIF | | | 1-Mar | CASSPGQVSYEQ | 1772 |
| | Naive | | | | | | | | | | | YF | 1940 |
| VF3 | Foreign_ | EBV_YVL | 0 | 0 | 0 | 0 | 2-Dec | CAVITGGGNKLTF | | | 1-May | CASSLAGGGEQY | 1754 |
| | Naive | | | | | | | | | | | F | 1941 |
| VG1 | Foreign_ | IV_GIL | 0 | 0 | 0 | 0 | 38- | CDPSGGNNRKLI | | | 19 | CASSVYSGGYNE | 1773 |
| | Naive | | | | | | 2/DV8 | W | | | | QFF | 1942 |
| VG2 | Foreign_ | 0 | 0 | 0 | 0 | 0 | 29/DV5 | CAATQGGSEKLV | | | 9 | CASSVGVGTDTQ | 1774 |
| | Naive | | | | | | | F | | | | YF | 1943 |
| VG3 | Foreign_ | EBV_YVL | 0 | 0 | 0 | 0 | | | | | 29-1 | CSVDNKAGGGYT | 1944 |
| | Naive | | | | | | | | | | | F | |
| VH1 | Foreign_ | HCV_A9N | 0 | 0 | 0 | 0 | 38- | CAYGSNNNDMR | | | 5-Jun | CASSYSPGTGNTI | 1775 |
| | Naive | | | | | | 2/DV8 | F | | | | YF | 1945 |
| VH2 | Foreign_ | YFV_LLW | 0 | 0 | 0 | 0 | 2-Dec | CAVSDDKIIF | | | 1-Jun | CASSEGPGQGSY | 1965 |
| | Naive | | | | | | | | | | | EQYF | 1946 |
| VH3 | Foreign_ | YFV_LLW | MART1_ | 0 | 0 | 0 | 2-Dec | CAVNNARLMF | | | | | 1776 |
| | Naive | | A2L | | | | | | | | | | |
| WA1 | Foreign_ | YFV_LLW | 0 | 0 | 0 | 0 | | | | | 2 | CASSEAFGRPNY | 1947 |
| | Naive | | | | | | | | | | | GYTF | |
| WA2 | Foreign_ | 0 | 0 | 0 | 0 | 0 | 3-Aug | CAVGAGPGAGS | | | 1-Jun | CASRSHPTYEQY | 1777 |
| | Naive | | | | | | | YQLTF | | | | F | 1948 |
| WA3 | Foreign_ | HSV_SLP | 0 | 0 | 0 | 0 | 14/DV4 | CAMREGTTDSW | | | 20-1 | CSARDLGLHQPQ | 1778 |
| | Naive | | | | | | | GKLQF | | | | HF | 1949 |
| WB1 | Foreign_ | YFV_LLW | 0 | 0 | 0 | 0 | 2-Dec | CAVDRDDKIIF | | | 27 | CASSFDLAGVNY | 1779 |
| | Naive | | | | | | | | | | | EQYF | 1950 |
| WB2 | Foreign_ | 0 | 0 | 0 | 0 | 0 | 17 | CASSGLSSGGSY | | | 1-Mar | CASSPLRGPADR | 1727 |
| | Naive | | | | | | | IPTF | | | | TGTEAFF | 1951 |
| WB3 | Foreign_ | CMV_MLN | 0 | 0 | 0 | 0 | 20 | CAVQAADSSASK | | | 2-Jul | CASSFWAGGWT | 1780 |
| | Naive | | | | | | | IIF | | | | EAFF | 1952 |
| WC1 | Foreign_ | YFV_LLW | 0 | 0 | 0 | 0 | | | | | 5-Jun | CASSYGSNYGYT | 1953 |
| | Naive | | | | | | | | | | | F | |
| WC2 | Foreign_ | HCV_LLF | 0 | 0 | 0 | 0 | 4 | CLVGGYSGGYQ | 3 | CAVRDMHPRGY | 15 | CATRGGEGQPQH | 1781 |
| | Naive | | | | | | | KVTF | | NKLIF | | F | 1884 |
| | | | | | | | | | | | | | 1954 |
| WC3 | Foreign_ | HTLV_LLF | HAFP_GLS | 0 | 0 | 0 | 3 | CAVRDYGNNRLA | | | | | 1782 |
| | Naive | | | | | | | F | | | | | |
| WD1 | Foreign_ | HCV_YLL | 0 | 0 | 0 | 0 | | | | | 1-Nov | CASSLGDWDLEA | 1955 |
| | Naive | | | | | | | | | | | FF | |
| WD2 | Foreign_ | YFV_LLW | 0 | 0 | 0 | 0 | 25 | CAGIDNAGNMLT | | | | | 1783 |
| | Naive | | | | | | | F | | | | | |
| WD3 | Foreign_ | YFV_LLW | 0 | 0 | 0 | 0 | 29/DV5 | CAAKDNRKLIW | | | 27 | CASGPGTAYGYT | 1784 |
| | Naive | | | | | | | | | | | F | 1956 |
| WE1 | Foreign_ | CMV_MLN | 0 | 0 | 0 | 0 | 4 | CLAFSGGYNKLIF | | | 27 | CASSLGPAYNEQ | 1785 |
| | Naive | | | | | | | | | | | FF | 1957 |
| WE2 | Foreign_ | YFV_LLW | 0 | 0 | 0 | 0 | 24 | CASSTDSWGKLQ | | | 2-Apr | CASSHDAGASTG | 1786 |
| | Naive | | | | | | | F | | | | ELFF | 1958 |
| WE3 | Foreign_ | YFV_LLW | 0 | 0 | 0 | 0 | | | | | 6-2, 6-3 | CASSSGAAYEQY | 1959 |
| | Naive | | | | | | | | | | | F | |
| WF1 | Foreign_ | CMV_MLN | 0 | 0 | 0 | 0 | 19 | CALSEAGYGNNR | | | 2 | CASSESFPASGG | 1787 |
| | Naive | | | | | | | LAF | | | | STDTQYF | 1960 |
| WF2 | Foreign_ | CMV_NLV | 0 | 0 | 0 | 0 | 3 | CAVNYGNMLTF | 14/DV4 | CAMRAFGADGQ | 5-Jun | CASSYATEVAGE | 1788 |
| | Naive | | | | | | | | | KLLF | | TQYF | 1885 |
| | | | | | | | | | | | | | 1961 |
| WF3 | Foreign_ | CMV_MLN | 0 | 0 | 0 | 0 | 14/DV4 | CAMREGMDSSY | | | 2 | CASMTNNQPQHF | 1789 |
| | Naive | | | | | | | KLIF | | | | | 1962 |
| WG1 | Foreign_ | YFV_LLW | 0 | 0 | 0 | 0 | 2-Dec | CAVIGSGKLIF | | | 1-Apr | CASSQTAGGYEQ | 1790 |
| | Naive | | | | | | | | | | | YF | 1963 |
| WG2 | Foreign_ | YFV_LLW | 0 | 0 | 0 | 0 | | | | | 9 | CASSVGGVSYNE | 1964 |
| | Naive | | | | | | | | | | | QFF | |
| WG3 | Foreign_ | EBV_YVL | 0 | 0 | 0 | 0 | 17 | CASSGLSSGGSY | | | 1-Mar | CASSPLRGPADR | 1727 |
| | Naive | | | | | | | IPTF | | | | TGTEAFF | 1951 |
| WH1 | Foreign_ | YFV_LLW | 0 | 0 | 0 | 0 | | | | | 2-Apr | CASSQVSSTGEL | 1965 |
| | Naive | | | | | | | | | | | FF | |
| WH2 | Foreign_ | CMV_MLN | 0 | 0 | 0 | 0 | 27 | CAGASSNTGKLIF | | | | | 1791 |
| | Naive | | | | | | | | | | | | |
| WH3 | Foreign_ | HBV_WLS | 0 | 0 | 0 | 0 | 2-Dec | CAVMADGQKLLF | | | 6-May | CASSQTIGTGFSN | 1792 |
| | Naive | | | | | | | | | | | EQFF | 1966 |
| TA7 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1967 |
| TA8 | Foreign_ | CMV_NLV | 0 | 0 | 0 | 0 | 35 | CAAPRETSYDKVI | | | | | 1794 |
| | Nonnaive | | | | | | | F | | | | | |
| TA9 | Foreign_ | CMV_NLV | 0 | 0 | 0 | 0 | 35 | CAAPRETSYDKVI | | | | | 1794 |
| | Nonnaive | | | | | | | F | | | | | |
| TB7 | Foreign_ | CMV_NLV | 0 | 0 | 0 | 0 | 26-2 | CILSNNNDMRF | | | | | 1793 |
| | Nonnaive | | | | | | | | | | | | |
| TB8 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| TB9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| TC7 | Foreign_ | CMV_NLV | 0 | 0 | 0 | 0 | 26-2 | CILSNNNDMRF | | | | | 1793 |
| | Nonnaive | | | | | | | | | | | | |
| TC8 | Foreign_ | CMV_NLV | 0 | 0 | 0 | 0 | | | | | | | |
| | Nonnaive | | | | | | | | | | | | |
| TC9 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| TD7 | Foreign_ | CMV_NLV | 0 | 0 | 0 | 0 | 35 | CAGPTKTSYDKVI | | | 12-3, | CASSSANYGYTF | 1795 |
| | Nonnaive | | | | | | | F | | | 12-4 | | 1968 |
| TD8 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| TD9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| TE7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| TE8 | Foreign_ | CMV_NLV | 0 | 0 | 0 | 0 | 35 | CAGPTKTSYDKVI | | | 12-3, | CASSSANYGYTF | 1795 |
| | Nonnaive | | | | | | | F | | | 12-4 | | 1968 |
| TE9 | Foreign_ | EBV_YVL | 0 | 0 | 0 | 0 | 17 | CATGLNYGGSQ | | | 2-Oct | CASSLFNQETQY | 1796 |
| | Nonnaive | | | | | | | GNLIF | | | | F | 1969 |
| TF7 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1967 |
| TF8 | Foreign_ | CMV_NLV | 0 | 0 | 0 | 0 | 3 | CAVFYGNKLVF | | | 5-Jun | CASSYATGIPDTQ | 1797 |
| | Nonnaive | | | | | | | | | | | YF | 1970 |
| TF9 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| TG7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| TG8 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| TG9 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1967 |
| TH7 | Foreign_ | 0 | 0 | 0 | 0 | 0 | | | | | | | |
| | Nonnaive | | | | | | | | | | | | |
| TH8 | Foreign_ | CMV_NLV | 0 | 0 | 0 | 0 | | | | | | | |
| | Nonnaive | | | | | | | | | | | | |
| TH9 | Foreign_ | EBV_YVL | 0 | 0 | 0 | 0 | | | | | | | |
| | Nonnaive | | | | | | | | | | | | |
| UA7 | Foreign_ | CMV_NLV | 0 | 0 | 0 | 0 | | | | | | | |
| | Nonnaive | | | | | | | | | | | | |
| UA8 | Foreign_ | CMV_NLV | 0 | 0 | 0 | 0 | 3 | CAVFYGNKLVF | | | 5-Jun | CASSYATGIPDTQ | 1797 |
| | Nonnaive | | | | | | | | | | | YF | 1970 |
| UA9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UB7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UB8 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1767 |
| UB9 | Foreign_ | CMV_NLV | 0 | 0 | 0 | 0 | | | | | 12-3, | CASSSANYGYTF | 1968 |
| | Nonnaive | | | | | | | | | | 12-4 | | |
| UC7 | Foreign_ | EBV_YVL | 0 | 0 | 0 | 0 | | | | | 1-Mar | CASSPLRGPADR | 1951 |
| | Nonnaive | | | | | | | | | | | TGTEAFF | |
| UC8 | Foreign_ | CMV_NLV | 0 | 0 | 0 | 0 | | | | | | | |
| | Nonnaive | | | | | | | | | | | | |
| UC9 | Foreign_ | CMV_NLV | 0 | 0 | 0 | 0 | | | | | | | |
| | Nonnaive | | | | | | | | | | | | |
| UD7 | Foreign_ | CMV_NLV | 0 | 0 | 0 | 0 | 26-2 | CILSNNNDMRF | | | | | 1793 |
| | Nonnaive | | | | | | | | | | | | |
| UD8 | Foreign_ | CMV_NLV | 0 | 0 | 0 | 0 | | | | | | | |
| | Nonnaive | | | | | | | | | | | | |
| UD9 | Foreign_ | CMV_NLV | 0 | 0 | 0 | 0 | | | | | 12-3, | CASSSANYGYTF | 1968 |
| | Nonnaive | | | | | | | | | | 12-4 | | |
| UE7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UE8 | Foreign_ | CMV_NLV | 0 | 0 | 0 | 0 | 3 | CAVFYGNKLVF | | | 5-Jun | CASSYATGIPDTQ | 1797 |
| | Nonnaive | | | | | | | | | | | YF | 1970 |
| UE9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UF7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UF8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UF9 | Foreign_ | CMV_NLV | 0 | 0 | 0 | 0 | | | | | | | |
| | Nonnaive | | | | | | | | | | | | |
| UG7 | Foreign_ | 0 | 0 | 0 | 0 | 0 | 4-Aug | CAVSDLNYGQNF | | | 9-Jul | CASTYGGGALNE | 1798 |
| | Nonnaive | | | | | | | VF | | | | QFF | 1971 |
| UG8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| UG9 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1967 |
| UH7 | Foreign_ | CMV_NLV | 0 | 0 | 0 | 0 | 26-2 | CILSNNNDMRF | | | | | 1793 |
| | Nonnaive | | | | | | | | | | | | |
| UH8 | Foreign_ | EBV_YVL | 0 | 0 | 0 | 0 | | | | | 1-Mar | CASSPLRGPADR | 1951 |
| | Nonnaive | | | | | | | | | | | TGTEAFF | |
| UH9 | Foreign_ | CMV_NLV | 0 | 0 | 0 | 0 | 3 | CAVFYGNKLVF | | | 5-Jun | CASSYATGIPDTQ | 1797 |
| | Nonnaive | | | | | | | | | | | YF | 1970 |
| VA7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VA8 | Foreign_ | CMV_NLV | 0 | 0 | 0 | 0 | 35 | CAGPTKTSYDKVI | | | 12-3, | CASSSANYGYTF | 1795 |
| | Nonnaive | | | | | | | F | | | 12-4 | | 1968 |
| VA9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VB7 | Foreign_ | CMV_NLV | 0 | 0 | 0 | 0 | 35 | CAAPRETSYDKVI | | | | | 1794 |
| | Nonnaive | | | | | | | F | | | | | |
| VB8 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1967 |
| VB9 | Foreign_ | CMV_NLV | 0 | 0 | 0 | 0 | 26-2 | CILSNNNDMRF | | | | | 1793 |
| | Nonnaive | | | | | | | | | | | | |
| VC7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VC8 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| VC9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VD7 | Foreign_ | CMV_NLV | 0 | 0 | 0 | 0 | | | | | 12-3, | CASSSANYGYTF | 1968 |
| | Nonnaive | | | | | | | | | | 12-4 | | |
| VD8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VD9 | Foreign_ | CMV_NLV | 0 | 0 | 0 | 0 | | | | | | | |
| | Nonnaive | | | | | | | | | | | | |
| VE7 | Foreign_ | CMV_NLV | 0 | 0 | 0 | 0 | | | | | | | |
| | Nonnaive | | | | | | | | | | | | |
| VE8 | Foreign_ | CMV_NLV | 0 | 0 | 0 | 0 | 3 | CAVFYGNKLVF | | | 5-Jun | CASSYATGIPDTQ | 1797 |
| | Nonnaive | | | | | | | | | | | YF | 1970 |
| VE9 | Foreign_ | CMV_NLV | 0 | 0 | 0 | 0 | 35 | CAAPRETSYDKVI | | | | | 1794 |
| | Nonnaive | | | | | | | F | | | | | |
| VF7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VF8 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1767 |
| VF9 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| VG7 | Foreign_ | EBV_YVL | 0 | 0 | 0 | 0 | 17 | CASSGLSSGGSY | | | 1-Mar | CASSPLRGPADR | 1727 |
| | Nonnaive | | | | | | | IPTF | | | | TGTEAFF | 1951 |
| VG8 | Foreign_ | CMV_NLV | 0 | 0 | 0 | 0 | 24 | CARNTGNQFYF | | | | | 1734 |
| | Nonnaive | | | | | | | | | | | | |
| VG9 | Foreign_ | CMV_NLV | 0 | 0 | 0 | 0 | | | | | 12-3, | CASSSANYGYTF | 1968 |
| | Nonnaive | | | | | | | | | | 12-4 | | |
| VH7 | Foreign_ | CMV_NLV | 0 | 0 | 0 | 0 | 35 | CAGPTKTSYDKVI | | | | | 1795 |
| | Nonnaive | | | | | | | F | | | | | |
| VH8 | Foreign_ | CMV_NLV | 0 | 0 | 0 | 0 | | | | | 12-3, | CASSSANYGYTF | 1968 |
| | Nonnaive | | | | | | | | | | 12-4 | | |
| VH9 | Foreign_ | CMV_NLV | 0 | 0 | 0 | 0 | | | | | | | |
| | Nonnaive | | | | | | | | | | | | |
| WA7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WA8 | Foreign_ | 0 | 0 | 0 | 0 | 0 | 17 | CASSGLSSGGSY | | | | | 1727 |
| | Nonnaive | | | | | | | IPTF | | | | | |
| WB7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WB8 | Foreign_ | CMV_NLV | 0 | 0 | 0 | 0 | 26-2 | CILSNNNDMRF | | | | | 1793 |
| | Nonnaive | | | | | | | | | | | | |
| WC7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WC8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WD7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WD8 | Foreign_ | CMV_NLV | 0 | 0 | 0 | 0 | | | | | 12-3, | CASSSANYGYTF | 1968 |
| | Nonnaive | | | | | | | | | | 12-4 | | |
| WE7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WE8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WF7 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WF8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WG7 | Foreign_ | CMV_NLV | 0 | 0 | 0 | 0 | 26-2 | CILSNNNDMRF | | | 30 | CAWSISDSSRVE | 1793 |
| | Nonnaive | | | | | | | | | | | AFF | 1967 |
| WG8 | Foreign_ | CMV_NLV | 0 | 0 | 0 | 0 | 35 | CAAPRETSYDKVI | | | 12-3, | CASSSANYGYTF | 1794 |
| | Nonnaive | | | | | | | F | | | 12-4 | | 1968 |
| WH7 | Foreign_ | CMV_NLV | 0 | 0 | 0 | 0 | 3 | CAVFYGNKLVF | | | 5-Jun | CASSYATGIPDTQ | 1797 |
| | Nonnaive | | | | | | | | | | | YF | 1970 |
| WH8 | Foreign_ | 0 | 0 | 0 | 0 | 0 | | | | | | | |
| | Nonnaive | | | | | | | | | | | | |
| TA4 | Self_Naive | TYR_YMD | 0 | 0 | 0 | 0 | 2-Dec | CAVNMFSNYGQ | | | | | 1799 |
| | | | | | | | | NFVF | | | | | |
| TA5 | Self_Naive | DRIP_MLY | 0 | 0 | 0 | 0 | 2-Sep | CALRIGGSTLGRL | | | 12-3, | CASSASGGRDYG | 1800 |
| | | | | | | | | YF | | | 12-4 | YTF | 1972 |
| TA6 | Self_Naive | DRIP_MLY | 0 | 0 | 0 | 0 | 1-Dec | CVVNLPNTGFQK | | | 12-3, | CASRTGTSGGFP | 1801 |
| | | | | | | | | LVF | | | 12-4 | NTGELFF | 1973 |
| TB4 | Self_Naive | MART1_A2L | 0 | 0 | 0 | 0 | 2-Dec | CAVNVANDMRF | | | 5-Jun | CASSYSIGNTEAF | 1802 |
| | | | | | | | | | | | | F | 1974 |
| TB5 | Self_Naive | MART1_A2L | 0 | 0 | 0 | 0 | 2-Dec | CAVNGGGKLTF | 16 | CAPTIYNQGGKLI | 24-1 | CATSGSYEQYF | 1803 |
| | | | | | | | | | | F | | | 1886 |
| | | | | | | | | | | | | | 1975 |
| TB6 | Self_Naive | PP1_SII | 0 | 0 | 0 | 0 | 2-Sep | CALPNFGNEKLT | | | 5-Jun | CASSYRFDSPLHF | 1804 |
| | | | | | | | | F | | | | | 1976 |
| TC4 | Self_Naive | ZNT8_LLI | 0 | 0 | 0 | 0 | 16 | CALSGSDSWGKL | | | 1-Oct | CASSESTIVQGYN | 1805 |
| | | | | | | | | QF | | | | EQFF | 1977 |
| TC5 | Self_Naive | MART1_A2L | 0 | 0 | 0 | 0 | 2-Dec | CAADNYGQNFVF | | | 30 | CAWSVSGLGYGY | 1806 |
| | | | | | | | | | | | | TF | 1978 |
| TC6 | Self_Naive | DRIP_MLY | 0 | 0 | 0 | 0 | 19 | CALSENTGFQKL | | | | | |
| | | | | | | | | VF | | | | | 1807 |
| TD4 | Self_Naive | MART1_A2L | 0 | 0 | 0 | 0 | 2-Dec | CAAPGNTPLVF | | | 3-Oct | CAISETTGINEQFF | 1808 |
| | | | | | | | | | | | | | 1979 |
| TD5 | Self_Naive | ZNT8_VVT | 0 | 0 | 0 | 0 | 38- | | | | 30 | CAWSGFSRTEAF | 1809 |
| | | | | | | | 2/DV8 | CAYRSVPDMRF | | | | F | 1980 |
| TD6 | Self_Naive | TYR_YMD | 0 | 0 | 0 | 0 | 1-Dec | CVVNFPTNAGKS | | | 14 | CASSLGQGLSYE | 1810 |
| | | | | | | | | TF | | | | QYF | 1981 |
| TE4 | Self_Naive | IGRP_FLW | 0 | 0 | 0 | 0 | 38- | CAYRSALWGAQ | | | 2-Jul | CASSLAENSGNTI | 1811 |
| | | | | | | | 2/DV8 | KLVF | | | | YF | 1982 |
| TE5 | Self_Naive | MART1_A2L | 0 | 0 | 0 | 0 | | | | | | | |
| TE6 | Self_Naive | MART1_A2L | 0 | 0 | 0 | 0 | 2-Dec | CAVKDARLMF | | | | | 1812 |
| TF4 | Self_Naive | ZNT8_VVT | 0 | 0 | 0 | 0 | 38- | CSLANAGKSTF | | | 2-Nov | CASSLVGGITGEL | 1813 |
| | | | | | | | 2/DV8 | | | | | FF | 1983 |
| TF5 | Self_Naive | ZNT8_VVT | 0 | 0 | 0 | 0 | 3-Dec | CAMSDTNAGKST | | | 27 | CASSTSAGFSNQ | 1814 |
| | | | | | | | | F | | | | PQHF | 1984 |
| TF6 | Self_Naive | 0 | 0 | 0 | 0 | 0 | 24 | CAPDQTGANNLF | | | | | 1815 |
| | | | | | | | | F | | | | | |
| TG4 | Self_Naive | MART1_A2L | 0 | 0 | 0 | 0 | 27 | CAGLNNARLMF | | | 2-Apr | CASSLQGGYGGG | 1816 |
| | | | | | | | | | | | | YTF | 1985 |
| TG5 | Self_Naive | MART1_A2L | 0 | 0 | 0 | 0 | 3-Dec | CAMTLSNFGNEK | | | 4-Jun | CASSDMAGDGYT | 1817 |
| | | | | | | | | LTF | | | | F | 1986 |
| TG6 | Self_Naive | AGL_GLI | 0 | 0 | 0 | 0 | 2-Dec | CAVGEYGNKLVF | | | 4-May | CASSPGPYEQYF | 1818 |
| | | | | | | | | | | | | | 1987 |
| TH4 | Self_Naive | MART1_A2L | 0 | 0 | 0 | 0 | 20 | CAVQTQGGSEKL | | | | | |
| | | | | | | | | VF | | | | | 1819 |
| TH5 | Self_Naive | MART1_A2L | IV_AIM | 0 | 0 | 0 | 20 | CAARGRDDKIIF | | | | | 1820 |
| TH6 | Self_Naive | MART1_A2L | 0 | 0 | 0 | 0 | 3-Aug | CAAFTSGNTPLV | | | 2-Nov | CASSLGGLGQPQ | 1821 |
| | | | | | | | | F | | | | HF | 1988 |
| UA4 | Self_Naive | GP100_YLE | 0 | 0 | 0 | 0 | | | | | 6-2, 6-3 | CASSWAPHYEQY | 1989 |
| | | | | | | | | | | | | F | |
| UA5 | Self_Naive | ZNT8_VVT | 0 | 0 | 0 | 0 | 2-Dec | CVFPNQGGSEKL | | | 1-Mar | CASSQDPGNGNT | 1822 |
| | | | | | | | | VF | | | | IYF | 1990 |
| UA6 | Self_Naive | 0 | 0 | 0 | 0 | 0 | 4-Aug | CAVSVITQGGSE | | | 3-Oct | CASSAGRYEQYF | 1823 |
| | | | | | | | | KLVF | | | | | 1991 |
| UB4 | Self_Naive | MART1_A2L | 0 | 0 | 0 | 0 | | | | | 27 | CASSVGGFGNQP | 1992 |
| | | | | | | | | | | | | QHF | |
| UB5 | Self_Naive | GP100_IMD | 0 | 0 | 0 | 0 | | | | | 15 | CATSTGWRTGTD | 1993 |
| | | | | | | | | | | | | TQYF | |
| UB6 | Self_Naive | MART1_A2L | IGRP_RLL | PPI_RLL | 0 | 0 | 2-Dec | CAVSSYDKVIF | | | 20-1 | CSALTGNQPQHF | 1824 |
| | | | | | | | | | | | | | 1994 |
| UC4 | Self_Naive | NYESO1_ | NYESO1_ | 0 | 0 | 0 | 38- | CALMDSNYQLIW | | | | | 1825 |
| | | 9A | V165 | | | | 2/DV8 | | | | | | |
| UC5 | Self_Naive | ZNT8_VMI | 0 | 0 | 0 | 0 | 2-Dec | CAVSGYSTLTF | | | 29-1 | CSVGLGQTGTEA | 1826 |
| | | | | | | | | | | | | FF | 1995 |
| UC6 | Self_Naive | MART1_A2L | 0 | 0 | 0 | 0 | 27 | CAGSGGGYQKV | 25 | CAGYKLVF | 5-Jun | CASSYSQGVYTG | 1827 |
| | | | | | | | | TF | | | | ELFF | 1887 |
| | | | | | | | | | | | | | 1996 |
| UD4 | Self_Naive | DDX5_YLL | 0 | 0 | 0 | 0 | | | | | 6-Jun | CASSWDYTEQYF | 1997 |
| UD5 | Self_Naive | ZNT8_LLS | 0 | 0 | 0 | 0 | 2-Dec | | | | 24-1 | CATSDSTGSYGY | 1828 |
| | | | | | | | | CAADSWGKLQF | | | | TF | 1998 |
| UD6 | Self_Naive | MART1_A2L | 0 | 0 | 0 | 0 | | | | | 5-Jun | CASLQGSGSPLH | 1999 |
| | | | | | | | | | | | | F | |
| UE4 | Self_Naive | 0 | 0 | 0 | 0 | 0 | | | | | 4-Jun | CASSVGGLGQPQ | 2000 |
| | | | | | | | | | | | | HF | |
| UE5 | Self_Naive | MART1_A2L | 0 | 0 | 0 | 0 | 2-Dec | CAAPSGNTPLVF | | | 19 | CASSMAGEQYF | 1829 |
| | | | | | | | | | | | | | 2001 |
| UE6 | Self_Naive | PP1_SII | 0 | 0 | 0 | 0 | 17 | CATDGEDDSWG | | | 4-May | CASVLGGSSYNE | 1830 |
| | | | | | | | | KLQF | | | | QFF | 2002 |
| UF4 | Self_Naive | MART1_A2L | 0 | 0 | 0 | 0 | 2-Dec | CAVGGGSQGNLI | | | 1-Mar | CASSPYRTGNIQY | 1831 |
| | | | | | | | | F | | | | F | 2003 |
| UF5 | Self_Naive | ZNT8_LLS | 0 | 0 | 0 | 0 | 2-Dec | CAVNPSNQFYF | | | 2 | CASRGPYHNEQF | 1832 |
| | | | | | | | | | | | | F | 2004 |
| UF6 | Self_Naive | MART1_A2L | 0 | 0 | 0 | 0 | 2-Dec | CAVNLNQAGTALI | | | 2-Apr | CASSQVGSTEAF | 1833 |
| | | | | | | | | F | | | | F | 2005 |
| UG4 | Self_Naive | MAGEA10_ | 0 | 0 | 0 | 0 | 17 | CATDEVDSSYKLI | | | 2 | | 1834 |
| | | GLY | | | | | | F | | | | CASTSYTEAFF | 2006 |
| UG5 | Self_Naive | MART1_A2L | 0 | 0 | 0 | 0 | 3-Dec | CAMSQSNFGNE | | | 18 | CASSPGQSPTNE | 1835 |
| | | | | | | | | KLTF | | | | KLFF | 2007 |
| UG6 | Self_Naive | TYR_YMD | 0 | 0 | 0 | 0 | 17 | CATGFSGAGSYQ | 41 | CAVEGSRLTF | 9-Jul | CASSLVMDNYGY | 1836 |
| | | | | | | | | LTF | | | | TF | 1863 |
| | | | | | | | | | | | | | 2008 |
| UH4 | Self_Naive | ZNT8_VVT | 0 | 0 | 0 | 0 | 3-Dec | CAMSDGGFQKLV | | | | | |
| | | | | | | | | F | | | | | 1837 |
| UH5 | Self_Naive | MART1_A2L | 0 | 0 | 0 | 0 | 2-Dec | | | | 27 | CASSSPGGETQY | 1838 |
| | | | | | | | | CAVNTGFQKLVF | | | | F | 2009 |
| UH6 | Self_Naive | MART1_A2L | 0 | 0 | 0 | 0 | 2-Dec | CAVNRDNFNKFY | | | 9-Jul | CASSPEPGSHEQ | 1839 |
| | | | | | | | | F | | | | YF | 2010 |
| VA4 | Self_Naive | MART1_A2L | 0 | 0 | 0 | 0 | 2-Dec | CAVNNNDMRF | | | | | 1840 |
| VA5 | Self_Naive | GP100_IMD | 0 | 0 | 0 | 0 | 3 | CAVRYSSASKIIF | | | 27 | CASRPGGGGYTF | 1841 |
| | | | | | | | | | | | | | 2011 |
| VA6 | Self_Naive | MART1_A2L | 0 | 0 | 0 | 0 | 2-Dec | CAAFSGGGADGL | | | 5-Jun | CASMRGAHTGEL | 1842 |
| | | | | | | | | TF | | | | FF | 2012 |
| VB4 | Self_Naive | MART1_A2L | 0 | 0 | 0 | 0 | | | | | | | |
| | | | | | | | | | | | 20-1 | CSASTGLTEAFF | 2013 |
| VB5 | Self_Naive | MART1_A2L | 0 | 0 | 0 | 0 | 2-Dec | CAVGGGYQKVTF | | | | | 1843 |
| VB6 | Self_Naive | MART1_A2L | 0 | 0 | 0 | 0 | | | | | 2-Nov | CASSLVRDLLFTD | 2014 |
| | | | | | | | | | | | | TQYF | |
| VC4 | Self_Naive | DRIP_MLY | 0 | 0 | 0 | 0 | 3-Dec | CAMSVGGLTGG | | | 12-3, | CASSLSGQGATN | 1844 |
| | | | | | | | | GNKLTF | | | 12-4 | EKLFF | 2015 |
| VC5 | Self_Naive | MART1_A2L | 0 | 0 | 0 | 0 | 2-Dec | CAVANAGNMLTF | | | 2-Apr | CASSQEVGLAGE | 1845 |
| | | | | | | | | | | | | TQYF | 2016 |
| VC6 | Self_Naive | MART1_A2L | 0 | 0 | 0 | 0 | 2-Dec | CAVNTGGGADGL | | | 28 | CASTQGDTGELF | 1846 |
| | | | | | | | | TF | | | | F | 2017 |
| VD4 | Self_Naive | DRIP_MLY | 0 | 0 | 0 | 0 | | | | | 12-3, | CASSSDRAGSPL | 2018 |
| | | | | | | | | | | | 12-4 | HF | |
| VD5 | Self_Naive | GFAP_NLA | GPC_FVG | 0 | 0 | 0 | 2-Jan | CAAYNAGNMLTF | | | 5-May | CASSHRGSGNTIY | 1847 |
| | | | | | | | | | | | | F | 2019 |
| VD6 | Self_Naive | HCHGA_TLS | | 0 | 0 | 0 | | | | | 1-May | CASSLADVGQYD | 2020 |
| | | | 0 | | | | | | | | | TDTQYF | |
| VE4 | Self_Naive | NYESO1_ | NYESO1_ | 0 | 0 | 0 | | | | | 2 | CASSGPARDTQY | 2021 |
| | | 9A | V165 | | | | | | | | | F | |
| VE5 | Self_Naive | MAGEC2_ | 0 | 0 | 0 | 0 | 2-Sep | CALSLAEGNFNK | | | 1-Jun | CASTWTGEQYF | 1848 |
| | | LLF | | | | | | FYF | | | | | 2022 |
| VE6 | Self_Naive | GP100_YLE | 0 | 0 | 0 | 0 | 17 | CAPGIAGGTSYG | | | 27 | CASSLAYSYEQYF | 1849 |
| | | | | | | | | KLTF | | | | | 2023 |
| VF4 | Self_Naive | MART1_A2L | 0 | 0 | 0 | 0 | | | | | 5-Jun | CASSYETGGSYE | 2024 |
| | | | | | | | | | | | | QYF | |
| VF5 | Self_Naive | MART1_A2L | 0 | 0 | 0 | 0 | 2-Dec | CAVPTFVNTGKLI | | | 28 | CASTYGGLNEQY | 1850 |
| | | | | | | | | F | | | | F | 2025 |
| VF6 | Self_Naive | GP100_IMD | GP100_ITD | 0 | 0 | 0 | 20 | CAVGRDGNQFYF | | | 19 | CASSTTGGGNYE | 1851 |
| | | | | | | | | | | | | QYF | 2026 |
| VG4 | Self_Naive | IGRP_VLF | 0 | 0 | 0 | 0 | 1-Aug | CAVNGDSGGSN | | | 1-Nov | CASSLWGAGELF | 1852 |
| | | | | | | | | YKLTF | | | | F | 2027 |
| VG5 | Self_Naive | MART1_A2L | 0 | 0 | 0 | 0 | 2 | CASNGGSYEQYF | | | | | 2028 |
| VG6 | Self_Naive | CD1_LLG | 0 | 0 | 0 | 0 | | | | | 27 | CASSLGDTEQFF | 2029 |
| VH4 | Self_Naive | ZNT8_LLS | 0 | 0 | 0 | 0 | 1-Dec | CVVSEEYTNAGK | | | 6-May | CASSLERLRVYS | 1853 |
| | | | | | | | | STF | | | | GYTF | 2030 |
| VH5 | Self_Naive | GP100_IMD | GP100_ | 0 | 0 | 0 | 14/DV4 | CAMREGTGRRAL | | | 2 | CATHGVSSRETQ | 1854 |
| | | | ITD | | | | | TF | | | | YF | 2031 |
| VH6 | Self_Naive | PP1_SII | 0 | 0 | 0 | 0 | 39 | CAGGGSQGNLIF | | | | | 1855 |
| WA4 | Self_Naive | HCHGA_TLS | 0 | 0 | 0 | 0 | | | | | | | |
| WA5 | Self_Naive | MART1_A2L | 0 | 0 | 0 | 0 | 2-Dec | CAVPTNFGNEKL | | | 6-May | CASSLEGTGLTDT | 1856 |
| | | | | | | | | TF | | | | QYF | 2032 |
| WA6 | Self_Naive | MART1_A2L | 0 | 0 | 0 | 0 | 2-Dec | CASTGGKLIF | | | 27 | CASSLSTVFTDTQ | 1857 |
| | | | | | | | | | | | | YF | 2033 |
| WB4 | Self_Naive | DRIP_MLY | 0 | 0 | 0 | 0 | 24 | CAVSSGTYKYIF | | | 12-3, | CASSLLGNTEAFF | 1858 |
| | | | | | | | | | | | 12-4 | | 2034 |
| WB5 | Self_Naive | GP100_IMD | GP100_ | 0 | 0 | 0 | 3 | CAVRDDTGGFKT | 8-3 | CAGGPYNTDKLIF | 19 | CASSTTEAYEQYF | 1859 |
| | | | ITD | | | | | IF | | | | | 1888 |
| | | | | | | | | | | | | | 2035 |
| WB6 | Self_Naive | GP100_IMD | GP100_ | 0 | 0 | 0 | 24 | CAFGDNYGQNFV | | | 19 | CASSTALAASYEQ | 1860 |
| | | | ITD | | | | | F | | | | YF | 2036 |
| WC4 | Self_Naive | MART1_A2L | 0 | 0 | 0 | 0 | | | | | 13 | CASSLGVGQPQH | 2037 |
| | | | | | | | | | | | | F | |
| WC5 | Self_Naive | GP100_IMD | GP100_ | 0 | 0 | 0 | 35 | CAGLADSNYQLI | | | 9 | CASSVGSGGRPS | 1861 |
| | | | ITD | | | | | W | | | | SYNEQFF | 2038 |
| WC6 | Self_Naive | ZNT8_LLS | 0 | 0 | 0 | 0 | 3-Dec | CAMDSSYKLIF | 26-1 | CIVRVECMYSGG | 9 | CASSALAGGQAD | #N/A |
| | | | | | | | | | | GADGLTF | | TQYF | |
| WD4 | Self_Naive | MART1_A2L | 0 | 0 | 0 | 0 | 41 | CAVEGSRLTF | | | 2-Nov | CASSSGPTMGGK | 1863 |
| | | | | | | | | | | | | LFF | 2040 |
| WD5 | Self_Naive | MART1_A2L | 0 | 0 | 0 | 0 | 2-Dec | CAVNPTGYSTLT | | | 2 | CASNSGGYNEQF | 1864 |
| | | | | | | | | F | | | | F | 2041 |
| WD6 | Self_Naive | MART1_A2L | 0 | 0 | 0 | 0 | 2-Dec | CALPKGGYSTLT | | | 5-Jun | CASSTTGTGLLEQ | 1865 |
| | | | | | | | | F | | | | YF | 2042 |
| WE4 | Self_Naive | 0 | 0 | 0 | 0 | 0 | 17 | CALNFGNEKLTF | | | 27 | CASSSGPRGNEQ | 1866 |
| | | | | | | | | | | | | FF | 2043 |
| WE5 | Self_Naive | MART1_A2L | 0 | 0 | 0 | 0 | 2-Dec | CAALTGNQFYF | | | 14 | CASSQGSGQPQH | 1867 |
| | | | | | | | | | | | | F | 2044 |
| WE6 | Self_Naive | MART1_A2L | 0 | 0 | 0 | 0 | 1-Dec | CVVNPFGNEKLT | | | 20-1 | CSARHPGVSTDT | 1868 |
| | | | | | | | | F | | | | QYF | 2045 |
| WF4 | Self_Naive | PP1_SII | 0 | 0 | 0 | 0 | 27 | CAGVPSNTGKLIF | | | 1-May | CASSPWRGPFQE | 1869 |
| | | | | | | | | | | | | TQYF | 2046 |
| WF5 | Self_Naive | DRIP_MLY | 0 | 0 | 0 | 0 | | | | | | | |
| WF6 | Self_Naive | SNPG_IML | 0 | 0 | 0 | 0 | | | | | 6-May | CASSPGKTEAFF | 2047 |
| WG4 | Self_Naive | SNPG_IML | 0 | 0 | 0 | 0 | | | | | 2-Oct | CASSESGRAEAF | 2048 |
| | | | | | | | | | | | | F | |
| WG5 | Self_Naive | MART1_A2L | 0 | 0 | 0 | 0 | 2-Dec | CAASLGGGADGL | | | 9-Jul | CASSPDVGHEKL | 1870 |
| | | | | | | | | TF | | | | FF | 2049 |
| WG6 | Self_Naive | MART1_A2L | 0 | 0 | 0 | 0 | 2-Dec | CALAIGFGNVLHC | | | 27 | CASSPIGGGSNE | 1871 |
| | | | | | | | | | | | | QFF | 2050 |
| WH4 | Self_Naive | GP100_IMD | GP100_ | 0 | 0 | 0 | 3 | CAVSFGSSNTGK | | | 5-Dec | CASGFTFQGSPE | 1872 |
| | | | ITD | | | | | LIF | | | | AFF | 2051 |
| WH5 | Self_Naive | MART1_A2L | 0 | 0 | 0 | 0 | | | | | | | |
| WH6 | Self_Naive | MART1_A2L | 0 | 0 | 0 | 0 | 2-Dec | CAASGGGADGLT | | | 28 | CASSFGGLARNE | 1873 |
| | | | | | | | | F | | | | QFF | 2052 |
| TA10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TA11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TA12 | Self_ | MART1_A2L | 0 | 0 | 0 | 0 | | | | | 6-2, 6-3 | CASSYFGGSLSE | 2053 |
| | Nonnaive | | | | | | | | | | | QYF | |
| TB10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TB11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TB12 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TC10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TC11 | Self_ | CD1_LLG | 0 | 0 | 0 | 0 | | | | | 27 | CASSFLTGTGELF | 2054 |
| | Nonnaive | | | | | | | | | | | F | |
| TC12 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TD10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TD11 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TD12 | Self_ | MART1_A2L | 0 | 0 | 0 | 0 | | | | | | | |
| | Nonnaive | | | | | | | | | | | | |
| TE10 | Self_ | DRIP_MLY | 0 | 0 | 0 | 0 | | | | | | | |
| | Nonnaive | | | | | | | | | | | | |
| TE11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TE12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TF10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TF11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TF12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TG10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TG11 | Self_ | MART1_A2L | 0 | 0 | 0 | 0 | 14/DV4 | CAMREGTGRRAL | | | | | 1854 |
| | Nonnaive | | | | | | | TF | | | | | |
| TG12 | Self_ | MART1_A2L | 0 | 0 | 0 | 0 | 12-2 | CAGNTGNQFYF | | | 28 | CASRPQGLGNTIY | 1874 |
| | Nonnaive | | | | | | | | | | | F | 2055 |
| TH10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TH11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| TH12 | Self_ | 0 | 0 | 0 | 0 | 0 | 14/DV4 | CAMREGTGRRAL | | | | | 1854 |
| | Nonnaive | | | | | | | TF | | | | | |
| UA10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UA11 | Self_ | MART1_A2L | ADI_SVA | PP1_SII | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UA12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UB10 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UB11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UB12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UC10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UC11 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UC12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UD10 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UD11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UD12 | Self_ | MART1_A2L | 0 | 0 | 0 | 0 | 14/DV4 | CAMREGTGRRAL | | | 6-2, 6-3 | CASSYFRGSLSE | 1854 |
| | Nonnaive | | | | | | | TF | | | | QYF | 2056 |
| UE10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UE11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UE12 | Self_ | MART1_ | 0 | 0 | 0 | 0 | 14/DV4 | CAMREGTGRRAL | | | 6-2, 6-3 | CASSYFGGSLSE | 1854 |
| | Nonnaive | A2L | | | | | | TF | | | | QYF | 2053 |
| UF10 | Self_ | MART1_A2L | ADI_SVA | 0 | 0 | 0 | | | | | 6-2, 6-3 | CASSYFGGSLSE | 2053 |
| | Nonnaive | | | | | | | | | | | QYF | |
| UF11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UF12 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UG10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UG11 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UG12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UH10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UH11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| UH12 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VA10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VA11 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VA12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VB10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VB11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VB12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VC10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VC11 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VC12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VD10 | Self_ | MART1_A2L | 0 | 0 | 0 | 0 | 12-2 | FAGGGGSSNTG | | | 9 | CASSPGGTEAFF | 1875 |
| | Nonnaive | | | | | | | KLIF | | | | | 2057 |
| VD11 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VD12 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VE10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VE11 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VE12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VF10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VF11 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VF12 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VG10 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VG11 | Self_ | MART1_A2L | 0 | 0 | 0 | 0 | 2-Dec | CAVTGQGGKLIF | | | 5-Jun | CASSFGGGGQPQ | 1879 |
| | Nonnaive | | | | | | | | | | | HF | 2058 |
| VG12 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| VH10 | Self_ | MART1_A2L | 0 | 0 | 0 | 0 | | | | | 6-2, 6-3 | CASSYFGGSLSE | 2053 |
| | Nonnaive | | | | | | | | | | | QYF | |
| VH11 | Self_ | DRIP_MLY | 0 | 0 | 0 | 0 | | | | | | | |
| | Nonnaive | | | | | | | | | | | | |
| VH12 | Self_ | MART1_A2L | 0 | 0 | 0 | 0 | 2-Dec | CAVGLGFGNVLH | | | 1-Apr | CASSLGVGTEAFF | 1877 |
| | Nonnaive | | | | | | | C | | | | | 2059 |
| WA10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WA9 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WB10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WB9 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WC10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WC9 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WD10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WD9 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WE10 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WE9 | Self_ | 0 | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WF10 | Self_ | MART1_A2L | ADI_SVA | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WF9 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WG10 | Self_ | DRIP_MLY | 0 | 0 | 0 | 0 | | | | | 12-3, | CASSFGRNRSQN | 2060 |
| | Nonnaive | | | | | | | | | | 12-4 | TEAFF | |
| WG9 | Self_ | MART1_A2L | 0 | 0 | 0 | 0 | 14/DV4 | CAMREGTGRRAL | | | 6-2, 6-3 | CASSYFGGSLSE | 1854 |
| | Nonnaive | | | | | | | TF | | | | QYF | 2053 |
| WH10 | Self_ | 0 | 0 | 0 | 0 | 0 | 14/DV4 | CAMREGPGGTS | | | 6-2, 6-3 | CASSYRQDSNQP | 1878 |
| | Nonnaive | | | | | | | YGKLTF | | | | QHF | 2061 |
| WH9 | Self_ | MART1_A2L | 0 | 0 | 0 | 0 | | | | | 6-2, 6-3 | CASSYFGGSLSE | 2053 |
| | Nonnaive | | | | | | | | | | | QYF | |

TABLE 5
Description of neoantigen and wildtype peptides used for experiment 3 and 4.

| | | Position | | | Wildtype HLA- | | | Mutant HLA- |
|---|---|---|---|---|---|---|---|---|
| Wildtype | Mutant | of | | SEQ | A2 Binding | | SEQ | A2 Binding |
| amino | amino | mutation | Wildtype | ID | NetMHC | Mutant | ID | NetMHC 4.0 |
| acid | acid | in peptide | peptide | NO | 4.0 (nM) | peptide | NO | (nM) |
| T | I | 3 | FLTYLDVSV | 2062 | 6.4 | FLIYLDVSV | 2082 | 4 |
| S | F | 1 | SMPDFDLHL | 2063 | 22.9 | FMPDFDLHL | 2083 | 5.5 |
| S | F | 8 | VLLGVKLSGV | 2064 | 32.5 | VLLGVKLFGV | 2084 | 9.1 |
| H | Y | 8 | ALIHHNTHL | 2065 | 79.3 | ALIHHNTYL | 2085 | 17.9 |
| L | F | 8 | VLENFTILLV | 2066 | 138.5 | VLENFTIFLV | 2086 | 50.6 |
| L | F | 9 | SVLENFTILL | 2067 | 182.7 | SVLENFTIFL | 2087 | 84.7 |
| A | V | 9 | ILTGLNYEA | 2068 | 41.7 | ILTGLNYEV | 2088 | 7.4 |
| S | F | 5 | ALYGSVPVL | 2069 | 15.3 | ALYGFVPVL | 2089 | 8.3 |
| L | M | 3 | VVLSWAPPV | 2070 | 9.6 | VVMSWAPPV | 2090 | 5.8 |
| L | P | 6 | ALLETLSLLL | 2071 | 35.7 | ALLETPSLLL | 2091 | 53.5 |
| L | H | 8 | ALSPVIPLI | 2072 | 8.1 | ALSPVIPHI | 2092 | 11.3 |
| H | Y | 8 | KLFEFLVHGV | 2073 | 4.4 | KLFEFLVYGV | 2093 | 3.3 |
| R | C | 4 | NLNRCSVPV | 2074 | 48.4 | NLNCCSVPV | 2094 | 18.2 |
| C | F | 5 | LIIPCIHLI | 2075 | 32.7 | LIIPFIHLI | 2095 | 24.5 |
| T | P | 6 | LLFGMTPCL | 2076 | 7.4 | LLFGMPPCL | 2096 | 11.7 |
| P | L | 6 | KLSHQPVLL | 2077 | 85.1 | KLSHQLVLL | 2097 | 25.8 |
| H | Y | 5 | AVGSHVYSV | 2078 | 91.5 | AVGSYVYSV | 2098 | 29.3 |
| P | L | 5 | FLYNPLTRV | 2079 | 4.4 | FLYNLLTRV | 2099 | 3.3 |
| Q | K | 8 | KLMNIQQQL | 2080 | 15.4 | KLMNIQQKL | 2100 | 20.3 |
| R | Q | 5 | MLGERLFPL | 2081 | 4 | MLGEQLFPL | 2101 | 3.4 |

TABLE 6
TetTCR-Seq summary for experiment 3.

| | Sorted | | | | | | | | | | | | SEQ ID |
|---|---|---|---|---|---|---|---|---|---|---|---|---|---|
| Cell | Popu- | Detected Peptide by MID Count | | | | | TCRα,1 | | TCRα,2 | | TCRβ | | NO |
| Name | lation | Rank 1 | Rank 2 | Rank 3 | Rank 4 | Rank 5 | TRAV | CDR3α | TRAV | CDR3α | TRBV | CDR3β | (L to R) |
| BA1 | Neo⁺WT⁺ | GANAB | GANAB- | 0 | 0 | 0 | | | | | 29-1*01 | CSVPEGNTGELF | 2312 |
| | | | S5F | | | | | | | | | F | |
| BA10 | Neo⁺WT⁺ | HCV-KLV | 0 | 0 | 0 | 0 | | | | | 7-9*01 | CASSLEGEQYF | 2313 |
| BA11 | Neo⁺WT⁺ | NSDHL- | NSDHL | 0 | 0 | 0 | 14/DV4*01 | CAMRESNTGGFK | | | | | 2102 |
| | | A9V | | | | | | TIF | | | | | |
| BA2 | Neo⁺WT⁺ | SMARCD3 | SMARCD3- | 0 | 0 | 0 | 5*01 | CAVYNTDKLIF | | | 4-1*01 | CASSQGALGYTF | 2103 |
| | | | H8Y | | | | | | | | | | 2314 |
| BA3 | Neo⁺WT⁺ | USP28 | 0 | 0 | 0 | 0 | 13- | GGTSYGKLTF | | | 12-3*01, | CASSFPDRGQGV | 2104 |
| | | | | | | | 2*01/13- | | | | 12-4*01 | YGYTF | 2315 |
| | | | | | | | 2*02 | | | | | | |
| BA6 | Neo⁺WT⁺ | FNDC3B- | FNDC3B | 0 | 0 | 0 | 12-2*01 | CAVNGQAGTALIF | | | | CASYFFALFTDTQ | 2105 |
| | | L3M | | | | | | | | | 25-1*01 | YF | 2316 |
| BA8 | Neo⁺WT⁺ | NSDHL | NSDHL- | 0 | 0 | 0 | | | | | | CSARLKGGGDTQ | 2317 |
| | | | A9V | | | | | | | | 20-1*01 | YF | |
| BA9 | Neo⁺WT⁺ | HCV-KLV | 0 | 0 | 0 | 0 | 38- | CAYTHARLMF | 21*01 | RINSGGSNYKL | 15*01 | CATSRDRGTDTF | 2106 |
| | | | | | | | 2/DV8*01 | | | TF | | F | 2289 |
| | | | | | | | | | | | | | 2318 |
| BB1 | Neo⁺WT⁺ | FNDC3B | FNDC3B- | 0 | 0 | 0 | 12-2*01 | CAGIPDAGGTSYG | | | 6-2*01, | CASSYSSDFWGD | 2107 |
| | | | L3M | | | | | KLTF | | | 6-3*01 | QPQHF | 2319 |
| BB10 | Neo⁺WT⁺ | MLL2 | MLL2-L8H | 0 | 0 | 0 | 12-2*01 | CAVNKPGFGNEKL | | | 27*01 | CASSGAAGTSAY | 2108 |
| | | | | | | | | TF | | | | NEQFF | 2320 |
| BB11 | Neo⁺WT⁺ | SEC24A- | SEC24A | 0 | 0 | 0 | 25*01 | CAGRKTSYDKVIF | | | 4-3*01 | CASSYASTGTLN | 2109 |
| | | PSL | | | | | | | | | | YGYTF | 2321 |
| BB12 | Neo⁺WT⁺ | FNDC3B- | FNDC3B | 0 | 0 | 0 | 8-3*01 | CAVGATNNAGNM | | | 7-9*01 | CASSPDLNPYEQ | 2110 |
| | | L3M | | | | | | LTF | | | | YF | 2322 |
| BB6 | Neo⁺WT⁺ | HCV-KLV | 0 | 0 | 0 | 0 | | | | | 13*01 | CASSSQGETYEQ | 2323 |
| | | | | | | | | | | | | YF | |
| BB7 | Neo⁺WT⁺ | HCV-KLV | 0 | 0 | 0 | 0 | 38- | CAYWEGAQKLVF | | | 15*01 | CATAKEGLAYEQ | 2111 |
| | | | | | | | 2/DV8*01 | | | | | FF | 2324 |
| BB8 | Neo⁺WT⁺ | FNDC3B | FNDC3B- | 0 | 0 | 0 | 8-1*01 | CAVNVYNQGGKLI | | | 13*01 | CASSSGLAGGPK | 2112 |
| | | | L3M | | | | | F | | | | HYEQYF | 2325 |
| BC1 | Neo⁺WT⁺ | NSDHL- | NSDHL | 0 | 0 | 0 | 14/DV4*01 | CAMSVSDTGNQF | | | 15*01 | CATSRDRGLTEA | 2113 |
| | | A9V | | | | | | YF | | | | FF | 2326 |
| BC10 | Neo⁺WT⁺ | FNDC3B | FNDC3B- | 0 | 0 | 0 | 8-3*01 | CAVGAGSNFGNE | | | 6-5*01 | CASSYGGNSPLH | 2114 |
| | | | L3M | | | | | KLTF | | | | F | 2327 |
| BC11 | Neo⁺WT⁺ | AKAP13 | AKAP13- | 0 | 0 | 0 | | | | | 29-1*01 | CSADVGGQNEQ | 2328 |
| | | | Q8K | | | | | | | | | YF | |
| BC12 | Neo⁺WT⁺ | WDR46 | WDR46- | 0 | 0 | 0 | 21*01 | CAVRNRDDKIIF | | | 9*01 | CASSVGTGYEQY | 2115 |
| | | | T3I | | | | | | | | | F | 2329 |
| BC2 | Neo⁺WT⁺ | 0 | 0 | 0 | 0 | 0 | | | | | 12-3*01, | CASSLSSRSNQP | 2330 |
| | | | | | | | | | | | 12-4*01 | QHF | |
| BC4 | Neo⁺WT⁺ | FNDC3B | 0 | 0 | 0 | 0 | 19*01 | CALSEVGAGSYQL | | | | | 2116 |
| | | | | | | | | TF | | | | | |
| BC5 | Neo⁺WT⁺ | FNDC3B | FNDC3B- | 0 | 0 | 0 | 3*01 | CAVQAGGYQKVT | | | 13*01 | CASSSRQGAGDT | 2117 |
| | | | L3M | | | | | F | | | | QYF | 2331 |
| BC8 | Neo⁺WT⁺ | NSDHL- | NSDHL | EMPTY | 0 | 0 | 14/DV4*01 | CAMREGNTGGFK | | | 9*01 | CASSAGGDTEAF | 2118 |
| | | A9V | | | | | | TIF | | | | F | 2332 |
| BC9 | Neo⁺WT⁺ | HCV-KLV | 0 | 0 | 0 | 0 | 38- | CAYGANDMRF | | | 25-1*01 | CASSDGGKDGYT | 2119 |
| | | | | | | | 2/DV8*01 | | | | | F | 2333 |
| BD1 | Neo⁺WT⁺ | FNDC3B | FNDC3B- | 0 | 0 | 0 | | | | | 28*01 | CASSLWRGMGA | 2334 |
| | | | L3M | | | | | | | | | GELFF | |
| BD10 | Neo⁺WT⁺ | FNDC3B | FNDC3B- | 0 | 0 | 0 | 29/DV5*01 | CAASATGGTSYGK | | | 19*01 | CASSFTSGSGHE | 2120 |
| | | | L3M | | | | | LTF | | | | QYF | 2335 |
| BD11 | Neo⁺WT⁺ | ERBB2 | ERBB2- | 0 | 0 | 0 | 14/DV4*01 | CAMHRDDKIIF | | | 12-3*01, | CASSLAVQRPSG | 2121 |
| | | | H8Y | | | | | | | | 12-4*01 | NTIYF | 2336 |
| BD12 | Neo⁺WT⁺ | NSDHL | NSDHL- | 0 | 0 | 0 | 38- | CASGIQGAQKLVF | | | 7-9*01 | CASSLSGVAYGY | 2122 |
| | | | A9V | | | | 2/DV8*01 | | | | | TF | 2337 |
| BD2 | Neo⁺WT⁺ | 0 | 0 | 0 | 0 | 0 | 24*01 | CALSGYSTLTF | | | 2*01 | CASSQGQGSSQ | 2123 |
| | | | | | | | | | | | | YF | 2338 |
| BD3 | Neo⁺WT⁺ | FNDC3B | FNDC3B- | 0 | 0 | 0 | 12-2*01 | CAVSKEGSYIPTF | | | 29-1*01 | CSVRGGGDNSPL | 2124 |
| | | | L3M | | | | | | | | | HF | 2339 |
| BD5 | Neo⁺WT⁺ | NSDHL- | NSDHL | 0 | 0 | 0 | 38-1*01 | CAFMIDNNNDMRF | | | 9*01 | CASSGLAGGPFG | 2125 |
| | | A9V | | | | | | | | | | METQYF | 2340 |
| BD8 | Neo⁺WT⁺ | SMARCD3 | 0 | 0 | 0 | 0 | 8-1*01 | CAVNAWNNDMRF | | | 27*01 | CASTQGGVDTQY | 2126 |
| | | | | | | | | | | | | F | 2341 |
| BE1 | Neo⁺WT⁺ | FNDC3B | FNDC3B- | 0 | 0 | 0 | 8-3*01 | CAVGGEAQGAQK | | | 7-7*01 | CASSWGPGYEQ | 2127 |
| | | | L3M | | | | | LVF | | | | YF | 2342 |
| BE10 | Neo⁺WT⁺ | HCV-KLV | 0 | 0 | 0 | 0 | 38-1*01 | CAVSGAGSYQLTF | | | 7-9*01 | CASSLVDSGLYE | 2128 |
| | | | | | | | | | | | | QYF | 2343 |
| BE12 | Neo⁺WT⁺ | FNDC3B | FNDC3B- | 0 | 0 | 0 | 19*01 | CALSEAMTSGTYK | | | 20-1*01 | CSAREVRDLYNE | 2129 |
| | | | L3M | | | | | YIF | | | | QFF | 2344 |
| BE3 | Neo⁺WT⁺ | FNDC3B | FNDC3B- | 0 | 0 | 0 | 3*01 | CAVSNLMDTGRR | | | 5-8*01 | CASSLSSGPYNE | 2130 |
| | | | L3M | | | | | ALTF | | | | QFF | 2345 |
| BE5 | Neo⁺WT⁺ | NSDHL | NSDHL- | 0 | 0 | 0 | 9-2*01 | CALSEHDMRF | | | 7-9*01 | CASSLPGGPRET | 2131 |
| | | | A9V | | | | | | | | | QYF | 2346 |
| BE9 | Neo⁺WT⁺ | FNDC3B | FNDC3B- | 0 | 0 | 0 | 17*01 | CATDADTGNQFYF | | | 12-3*01, | CASSFGPYGYTF | 2132 |
| | | | L3M | | | | | | | | 12-4*01 | | 2347 |
| BF11 | Neo⁺WT⁺ | NSDHL- | NSDHL | 0 | 0 | 0 | 38- | CALNTGTASKLTF | | | 7-8*01 | CASSLQASGRET | 2133 |
| | | A9V | | | | | 2/DV8*01 | | | | | QYF | 2348 |
| BF12 | Neo⁺WT⁺ | HCV-KLV | EMPTY | 0 | 0 | 0 | 38- | CAYYGGGATNKLI | | | 13*01 | CASSLGSGTQYF | 2134 |
| | | | | | | | 2/DV8*01 | F | | | | | 2349 |
| BF3 | Neo⁺WT⁺ | FNDC3B | FNDC3B- | 0 | 0 | 0 | 14/DV4*01 | CAMSLRSYTGNQF | | | 20-1*01 | CSARISTSSSYEQ | 2135 |
| | | | L3M | | | | | YF | | | | YF | 2350 |
| BF5 | Neo⁺WT⁺ | FNDC3B | FNDC3B- | 0 | 0 | 0 | 29/DV5*01 | CAAKDNYGQNFVF | | | 5-5*01 | CASSLYGGESQE | 2136 |
| | | | L3M | | | | | | | | | TQYF | 2351 |
| BF9 | Neo⁺WT⁺ | FNDC3B- | FNDC3B | 0 | 0 | 0 | | | | | | | |
| | | L3M | | | | | | | | | | | |
| BG1 | Neo⁺WT⁺ | MRM1 | MRM1- | 0 | 0 | 0 | 29/DV5*01 | CAASLRYFGNEKL | 13- | CAVVMEYGNKL | 12-3*01, | CASSPDPYEQYF | 2137 |
| | | | T6P | | | | | TF | 2*01 | VF | 12-4*01 | | 2290 |
| | | | | | | | | | | | | | 2352 |
| BG10 | Neo⁺WT⁺ | PGM5 | PGM5- | 0 | 0 | 0 | 41*01 | CAVTDYNTDKLIF | | | 28*01 | CASSFRGEAFF | 2138 |
| | | | H5Y | | | | | | | | | | 2353 |
| BG11 | Neo⁺WT⁺ | HCV-KLV | 0 | 0 | 0 | 0 | 38- | CAYVEGNYQLIW | | | 19*01 | CASSIAAGNTIYF | 2139 |
| | | | | | | | 2/DV8*01 | | | | | | 2354 |
| BG12 | Neo⁺WT⁺ | 0 | 0 | 0 | 0 | 0 | 19*01 | CALSEVRYSSASKI | | | 27*01 | CASSLHREVNEK | 2140 |
| | | | | | | | | IF | | | | LFF | 2355 |
| BG2 | Neo⁺WT⁺ | FNDC3B | FNDC3B- | 0 | 0 | 0 | 19*01 | CALRGRVAGANNL | | | 6-1*01 | CASSEWAGQPQ | 2141 |
| | | | L3M | | | | | FF | | | | HF | 2356 |
| BG5 | Neo⁺WT⁺ | NSDHL | NSDHL- | 0 | 0 | 0 | 8-3*01 | CAVAFNNAGNMLT | | | 5-4*01 | CASSGGGAEAFF | 2142 |
| | | | A9V | | | | | F | | | | | 2357 |
| BG7 | Neo⁺WT⁺ | ERBB2 | ERBB2- | 0 | 0 | 0 | 10*01 | CVVSGVNVWGTY | | | 19*01 | CASSIESGSKQR | 2143 |
| | | | H8Y | | | | | KYIF | | | | NEQFF | 2358 |
| BG9 | Neo⁺WT⁺ | 0 | 0 | 0 | 0 | 0 | | | | | 10-3*01 | CATREPPNTEAF | 2359 |
| | | | | | | | | | | | | F | |
| BH1 | Neo⁺WT⁺ | AKAP13 | AKAP13- | 0 | 0 | 0 | 24*01 | CAFPMDSNYQLIW | | | 6-6*01 | CASSYNSMNTEA | 2144 |
| | | | Q8K | | | | | | | | | FF | 2360 |
| BH10 | Neo⁺WT⁺ | FNDC3B | FNDC3B- | 0 | 0 | 0 | 12-2*01 | CAPGGEKLTF | | | 7-6*01 | CASSLGGPAEQY | 2145 |
| | | | L3M | | | | | | | | | F | 2361 |
| BH11 | Neo⁺WT⁺ | HCV-KLV | 0 | 0 | 0 | 0 | 38-2/ | CSVGGGSEKLVF | | | 7-9*01 | CASSFGGYEQYF | 2146 |
| | | | | | | | DV8*01 | | | | | | 2362 |
| BH2 | Neo⁺WT⁺ | FNDC3B- | FNDC3B | 0 | 0 | 0 | | | | | 28*01 | CASSSSGIGETQ | 2363 |
| | | L3M | | | | | | | | | | YF | |
| BH5 | Neo⁺WT⁺ | MLL2 | MLL2-L8H | 0 | 0 | 0 | 12-2*01 | CAGLNSDGQKLLF | | | 6-2*01, | CASSYSSDRSSY | 2147 |
| | | | | | | | | | | | 6-3*01 | EQYF | 2364 |
| BH6 | Neo⁺WT⁺ | MLL2-L8H | GANAB | 0 | 0 | 0 | 12-2*01 | CAVNSKSGYSTLT | | | 6-2*01, | CASKSWDMAYE | 2148 |
| | | | | | | | | F | | | 6-3*01 | QYF | 2365 |
| BH7 | Neo⁺WT⁺ | HCV-KLV | 0 | 0 | 0 | 0 | 12-2*01 | CAVSMDTGRRALT | | | 28*01 | CASSSGTSLTLTY | 2149 |
| | | | | | | | | F | | | | NEQFF | 2366 |
| BH8 | Neo⁺WT⁺ | FNDC3B | FNDC3B- | 0 | 0 | 0 | 14/DV4*01 | CAMREGGNDMRF | | | 4-2*01 | CASSPRIGGPRE | 2150 |
| | | | L3M | | | | | | | | | GYTF | 2367 |
| BH9 | Neo⁺WT⁺ | FNDC3B- | FNDC3B | 0 | 0 | 0 | 3*01 | CAVRDKNRDDKIIF | | | 7-9*01 | CASQPWFNTGEL | 2151 |
| | | L3M | | | | | | | | | | FF | 2368 |
| CA5 | Neo⁺WT⁺ | NSDHL- | NSDHL | 0 | 0 | 0 | 5*01 | CAEKGAGGSYIPT | | | 10-3*01 | CAISPGGEQFF | 2152 |
| | | A9V | | | | | | F | | | | | 2369 |
| CA6 | Neo⁺WT⁺ | NSDHL | NSDHL- | 0 | 0 | 0 | 14/DV4*01 | CAMREVREAGNQ | | | 6-6*01 | CASSYSLAGEFF | 2153 |
| | | | A9V | | | | | FYF | | | | | 2370 |
| CB4 | Neo⁺WT⁺ | AKAP13 | AKAP13- | 0 | 0 | 0 | | | | | 3-1*01 | CASGKGDTEAFF | 2371 |
| | | | Q8K | | | | | | | | | | |
| CB5 | Neo⁺WT⁺ | HCV-KLV | 0 | 0 | 0 | 0 | 38- | CAYLDDYKLSF | | | 9*01 | CASSVETTDYGY | 2154 |
| | | | | | | | 2/DV8*01 | | | | | TF | 2372 |
| CB6 | Neo⁺WT⁺ | FNDC3B | FNDC3B- | 0 | 0 | 0 | 41*01 F | CAVRLDDFGNVLH | | | 7-2*01 | CASSFAPGQGIE | 2155 |
| | | | L3M | | | | | C | | | | KLFF | 2373 |
| CC11 | Neo⁺WT⁺ | FNDC3B | FNDC3B- | 0 | 0 | 0 | 14/DV4*01 | CAMREGPSQAGT | | | 2*01 | CASSEVSVLYEQ | 2156 |
| | | | L3M | | | | | ALIF | | | | YF | 2374 |
| CC6 | Neo⁺WT⁺ | MLL2-L8H | MLL2 | 0 | 0 | 0 | 14/DV4*01 | CALYGGSQGNLIF | 17*01 | CATASLRNYGQ | 9*01 | CASSVETGGLDT | 2157 |
| | | | | | | | | | | NFVF | | QYF | 2291 |
| | | | | | | | | | | | | | 2375 |
| CC9 | Neo⁺WT⁺ | NSDHL- | NSDHL | 0 | 0 | 0 | 14/DV4*01 | CAMRGSDGQKLL | | | 9*01 | CASSRGGGTEAF | 2158 |
| | | A9V | | | | | | F | | | | F | 2376 |
| CD12 | Neo⁺WT⁺ | NSDHL- | NSDHL | 0 | 0 | 0 | 14/DV4*01 | CAMREPYSGAGS | | | 9*01 | CASGAQHTEAFF | 2159 |
| | | A9V | | | | | | YQLTF | | | | | 2377 |
| CD3 | Neo⁺WT⁺ | HCV-KLV | 0 | 0 | 0 | 0 | 38- | CAYFELDMRF | 26- | CIVRVDERGTS | 5-5*01 | CASSFEGQETQY | 2160 |
| | | | | | | | 2/DV8*01 | | 1*01 | YGKLTF | | F | 2292 |
| | | | | | | | | | | | | | 2378 |
| CE8 | Neo⁺WT⁺ | HCV-KLV | 0 | 0 | 0 | 0 | | | | | | | |
| CF10 | Neo⁺WT⁺ | FNDC3B | FNDC3B- | 0 | 0 | 0 | | | | | 10-3*01 | CAISELGYEQYF | 2379 |
| | | | L3M | | | | | | | | | | |
| CG6 | Neo⁺WT⁺ | HCV-KLV | 0 | 0 | 0 | 0 | | | | | 20-1*01 | CSATSEYTEAFF | 2380 |
| CH10 | Neo⁺WT⁺ | HCV-KLV | 0 | 0 | 0 | 0 | 38-2/ | CAYSTGDMRF | | | 10-3*01 | CAISSDRTDEQYF | 2161 |
| | | | | | | | DV8*01 | | | | | | 2381 |
| CH6 | Neo⁺WT⁺ | EMPTY | GNL3L | PABPC1 | 0 | 0 | 12-1*01 | CVVSPYNQGGKLI | | | 7-9*01 | CASSLDIGDQPQ | 2162 |
| | | | | | | | | F | | | | HF | 2382 |
| CH8 | Neo⁺WT⁺ | 0 | 0 | 0 | 0 | 0 | | | | | | | |
| AA1 | Neo⁺WT⁻ | MLL2-L8H | 0 | 0 | 0 | 0 | 5*01 | CAESRGTDKLIF | | | 2*01 | CASSFMETQYF | 2163 |
| | | | | | | | | | | | | | 2383 |
| AA10 | Neo⁺WT⁻ | GNL3L- | 0 | 0 | 0 | 0 | 6*01 | CALQTGANNLFF | | | 27*01 | CASSLWAGETQY | 2164 |
| | | R4C | | | | | | | | | | F | 2384 |
| AA11 | Neo⁺WT⁻ | GNL3L- | 0 | 0 | 0 | 0 | 5*01 | CAEYSSASKIIF | | | 11-3*01 | CASSLDYNEQFF | 2165 |
| | | R4C | | | | | | | | | | | 2385 |
| AA12 | Neo⁺WT⁻ | SEC24A- | 0 | 0 | 0 | 0 | 1-1*01 | CAVLNSGNTPLVF | | | 12-3*01, | CASSPGRTQYF | 2166 |
| | | PSL | | | | | | | | | 12-4*01 | | 2386 |
| AA2 | Neo⁺WT⁻ | MLL2-L8H | 0 | 0 | 0 | 0 | 25*01 | CAGNYGGSQGNLI | 12- | CAVGAEYGNKL | 19*01 | CASSMAAGTHEQ | 2167 |
| | | | | | | | | F | 2*01 | VF | | YF | 2293 |
| | | | | | | | | | | | | | 2387 |
| AA3 | Neo⁺WT⁻ | GNL3L- | 0 | 0 | 0 | 0 | 26-2*01 | CILRDALIQAGNML | | | 20-1*01 | CSARTDRGNNYG | 2168 |
| | | R4C | | | | | | TF | | | | YTF | 2388 |
| AA4 | Neo⁺WT⁻ | GNL3L- | 0 | 0 | 0 | 0 | 12-2*01 | CAVNLNYGGSQG | | | 4-2*01 | CASSANQGYEQY | 2169 |
| | | R4C | | | | | | NLIF | | | | F | 2389 |
| AA5 | Neo⁺WT⁻ | GNL3L- | 0 | 0 | 0 | 0 | | | | | 2*01 | CASSEWGIEAFF | 2390 |
| | | R4C | | | | | | | | | | | |
| AA6 | Neo⁺WT⁻ | GNL3L- | 0 | 0 | 0 | 0 | 12-2*01 | CAVNPVQGAQKLV | | | 4-2*01 | CASSQVEGYEQY | 2170 |
| | | R4C | | | | | | F | | | | F | 2391 |
| AA7 | Neo⁺WT⁻ | GANAB- | 0 | 0 | 0 | 0 | 29/DV5*01 | CAASAGLAGSYQL | | | 6-1*01 | CASSEISSGGPFL | 2171 |
| | | S5F | | | | | | TF | | | | DTQYF | 2392 |
| AA8 | Neo⁺WT⁻ | GNL3L- | 0 | 0 | 0 | 0 | 13-1*01 | CAASEAF | | | 6-2*01, | CASSYSSRVNYE | 2172 |
| | | R4C | | | | | | | | | 6-3*01 | QYF | 2393 |
| AA9 | Neo⁺WT⁻ | 0 | 0 | 0 | 0 | 0 | 1-2*01 | CAVRESYGQNFVF | | | 7-6*01 | CASSSGLAGNST | 2173 |
| | | | | | | | | | | | | QYF | 2394 |
| AB1 | Neo⁺WT⁻ | 0 | 0 | 0 | 0 | 0 | 21*01 | CAVRGYSTLTF | | | 2*01 | CASTTGLEAFF | 2174 |
| | | | | | | | | | | | | | 2395 |
| AB10 | Neo⁺WT⁻ | GNL3L- | 0 | 0 | 0 | 0 | 22*01 | CAVVTTTDKLIF | | | 20-1*01 | CSARDLGGGGD | 2175 |
| | | R4C | | | | | | | | | | EQFF | 2396 |
| AB11 | Neo⁺WT⁻ | 0 | 0 | 0 | 0 | 0 | 12-2*01 | CAVMDDSWGKLQ | | | 5-1*01 | CASSLATGGGEQ | 2176 |
| | | | | | | | | F | | | | YF | 2397 |
| AB12 | Neo⁺WT⁻ | GNL3L- | 0 | 0 | 0 | 0 | 13-2*01 | CAVSNSGGSNYKL | | | 6-5*01 | CASSYGGISYGY | 2177 |
| | | R4C | | | | | | TF | | | | TF | 2398 |
| AB2 | Neo⁺WT⁻ | GNL3L- | 0 | 0 | 0 | 0 | 13-2*01 | CAETGQGGGADG | | | 6-5*01 | CASSYASGGYEQ | 2178 |
| | | R4C | | | | | | LTF | | | | YF | 2399 |
| AB3 | Neo⁺WT⁻ | NSDHL- | 0 | 0 | 0 | 0 | 3*01 | CAVRDMDNARLM | | | 9*01 | CASSVGGDIGIGY | 2179 |
| | | A9V | | | | | | F | | | | TF | 2400 |
| AB4 | Neo⁺WT⁻ | MRM1- | 0 | 0 | 0 | 0 | 3*01 | CAVRDQAGTALIF | | | 13*01 | CASSFGPVEQYF | 2180 |
| | | T6P | | | | | | | | | | | 2401 |
| AB5 | Neo⁺WT⁻ | GANAB- | 0 | 0 | 0 | 0 | 13-2*01 | CAETGDSNYQLIW | | | 15*01 | CATSEGLQYEQY | 2181 |
| | | S5F | | | | | | | | | | F | 2402 |
| AB6 | Neo⁺WT⁻ | GNL3L- | 0 | 0 | 0 | 0 | 22*01 | CAVRTSYDKVIF | | | 20-1*01 | CSVTQGDGTDTQ | 2182 |
| | | R4C | | | | | | | | | | YF | 2403 |
| AB7 | Neo⁺WT⁻ | PGM5- | 0 | 0 | 0 | 0 | 12-3*01 | CAMSATASGTYKY | | | 9*01 | CASSVEGAHPIQ | 2183 |
| | | H5Y | | | | | | IF | | | | ETQYF | 2404 |
| AB8 | Neo⁺WT⁻ | SEC24A- | 0 | 0 | 0 | 0 | 12-1*01 | CVVNQRGGGADG | | | 13*01 | CASSLGQTVTQE | 2184 |
| | | PSL | | | | | | LTF | | | | TQYF | 2405 |
| AB9 | Neo⁺WT⁻ | 0 | 0 | 0 | 0 | 0 | 19*01 | CALSEAENDYKLS | | | 3-1*01 | CASSQDLTASYY | 2185 |
| | | | | | | | | F | | | | NEQFF | 2406 |
| AC1 | Neo⁺WT⁻ | GNL3L- | 0 | 0 | 0 | 0 | 5*01 | CAESLRPGGGAD | | | 9*01 | CASSVAAGGAYE | 2186 |
| | | R4C | | | | | | GLTF | | | | QYF | 2407 |
| AC10 | Neo⁺WT⁻ | FNDC3B- | 0 | 0 | 0 | 0 | 20*01 | CAVQASSGAGSY | | | 4-2*01 | CASRGPYNEQFF | 2187 |
| | | L3M | | | | | | QLTF | | | | | 2408 |
| AC11 | Neo⁺WT⁻ | PGM5- | 0 | 0 | 0 | 0 | 8-3*01 | CAVGGGSQGNLIF | | | 13*01 | CASSLATEQFF | 2188 |
| | | H5Y | | | | | | | | | | | 2409 |
| AC12 | Neo⁺WT⁻ | SEC24A- | 0 | 0 | 0 | 0 | 16*01 | CALRDSSGGSYIP | 21*01 | CAVTWGHNNA | 7-6*01 | CASSLESTANTE | 2189 |
| | | PSL | | | | | | TF | | GNMLTF | | AFF | 2294 |
| | | | | | | | | | | | | | 2410 |
| AC2 | Neo⁺WT⁻ | GNL3L- | 0 | 0 | 0 | 0 | | | | | 5-8*01 | CASNPGPTYGYT | 2411 |
| | | R4C | | | | | | | | | | F | |
| AC3 | Neo⁺WT⁻ | GNL3L- | 0 | 0 | 0 | 0 | 10*01 | CVTGTNAGKSTF | 12- | CALLLGGGADG | 2*01 | CAIMSSGRADGE | 2190 |
| | | R4C | | | | | | | 2*01 | LTF | | LFF | 2295 |
| | | | | | | | | | | | | | 2412 |
| AC4 | Neo⁺WT⁻ | NSDHL- | 0 | 0 | 0 | 0 | 9-2*01 | CALSDSVNNAGN | | | 9*01 | CASSQGSDEQYF | 2191 |
| | | A9V | | | | | | MLTF | | | | | 2413 |
| AC5 | Neo⁺WT⁻ | GNL3L- | 0 | 0 | 0 | 0 | 26-1*01 | CIVRGDIKAAGNKL | 12- | CAMIGNSGNTP | 6-5*01 | CASSYGGAYEQY | 2192 |
| | | R4C | | | | | | TF | 3*01 | LVF | | F | 2296 |
| | | | | | | | | | | | | | 2414 |
| AC6 | Neo⁺WT⁻ | NSDHL- | 0 | 0 | 0 | 0 | 9-2*01 | CALADMNRDDKIIF | | | 9*01 | CASSVDPGQSYE | 2193 |
| | | A9V | | | | | | | | | | QYF | 2415 |
| AC7 | Neo⁺WT⁻ | WDR46- | 0 | 0 | 0 | 0 | 12-2*01 | CAVKGGGSYIPTF | | | | | 2194 |
| | | T3I | | | | | | | | | | | |
| AC8 | Neo⁺WT⁻ | GNL3L- | 0 | 0 | 0 | 0 | 20*01 | CAIHRGPGAGSYQ | | | 19*01 | CASSIVDGYEQY | 2195 |
| | | R4C | | | | | | LTF | | | | F | 2416 |
| AC9 | Neo⁺WT⁻ | NSDHL- | 0 | 0 | 0 | 0 | 14/DV4*01 | CAMREPDSNYQLI | 13- | CAENRNAGNN | 9*01 | CASSGFRGELFF | 2196 |
| | | A9V | | | | | | W | 2*01 | RKLIW | | | 2297 |
| | | | | | | | | | | | | | 2417 |
| AD1 | Neo⁺WT⁻ | GNL3L- | 0 | 0 | 0 | 0 | 12-2*01 | CAVYSSASKIIF | | | 6-5*01 | CASSYGQGYEQY | 2197 |
| | | R4C | | | | | | | | | | F | 2418 |
| AD10 | Neo⁺WT⁻ | MLL2-L8H | 0 | 0 | 0 | 0 | 19*01 | CALRENYNNNDM | 16*01 | CALSNAGNNRK | 3-1*01 | CASSQVGGSYPR | 2198 |
| | | | | | | | | RF | | LIW | | EQFF | 2298 |
| | | | | | | | | | | | | | 2149 |
| AD11 | Neo⁺WT⁻ | GNL3L- | 0 | 0 | 0 | 0 | 22*01 | CAVKTSYDKVIF | | | 28*01 | CASSRGGHEQYF | 2199 |
| | | R4C | | | | | | | | | | | 2420 |
| AD12 | Neo⁺WT⁻ | GNL3L- | 0 | 0 | 0 | 0 | 10*01 | CVVTTTGGGYNKL | 1-2*01 | CAVRDTGGGN | 2*01 | CASSDPNDYEQY | 2200 |
| | | R4C | | | | | | IF | | KLTF | | F | 2299 |
| | | | | | | | | | | | | | 2421 |
| AD2 | Neo⁺WT⁻ | GNL3L- | 0 | 0 | 0 | 0 | 13-1*01 | CAATPTNAGKSTF | | | 19*01 | CASSIVGQGYEQ | 2201 |
| | | R4C | | | | | | | | | | YF | 2422 |
| AD3 | Neo⁺WT⁻ | GNL3L- | 0 | 0 | 0 | 0 | 35*01 | CAGHNNNAGNML | | | | | 2202 |
| | | R4C | | | | | | TF | | | | | |
| AD4 | Neo⁺WT⁻ | MLL2-L8H | 0 | 0 | 0 | 0 | 12-3*01 | CASGEYYGQNFVF | | | 19*01 | CASSMGGVGTEA | 2203 |
| | | | | | | | | | | | | FF | 2423 |
| AD5 | Neo⁺WT⁻ | GNL3L- | 0 | 0 | 0 | 0 | 6*01 | CALSGYSTLTF | | | 4-2*01 | CASSPYSNQPQH | 2123 |
| | | R4C | | | | | | | | | | F | 2424 |
| AD6 | Neo⁺WT⁻ | GNL3L- | 0 | 0 | 0 | 0 | 22*01 | CAVKTSYDKVIF | | | | | 2199 |
| | | R4C | | | | | | | | | | | |
| AD7 | Neo⁺WT⁻ | MLL2-L8H | 0 | 0 | 0 | 0 | 12-2*01 | CAVGGYNFNKFYF | 12- | CVALRGGSQG | 5-6*01 | CASSFRDSSYEQ | 2204 |
| | | | | | | | | | 1*01 | NLIF | | YF | 2300 |
| | | | | | | | | | | | | | 2425 |
| AD8 | Neo⁺WT⁻ | GNL3L- | 0 | 0 | 0 | 0 | 19*01 | CALSEADTGGFKTI | | | 6-6*01 | CASSYSVKGQDY | 2205 |
| | | R4C | | | | | | F | | | | SYEQYF | 2426 |
| AD9 | Neo⁺WT⁻ | MLL2-L8H | 0 | 0 | 0 | 0 | 25*01 | CAGTGAGSYQLTF | | | 6-5*01 | CASRLHGGTPSY | 2206 |
| | | | | | | | | | | | | EQYF | 2427 |
| AE1 | Neo⁺WT⁻ | PGM5- | 0 | 0 | 0 | 0 | 3*01 | CAVRDMQDSNYQ | | | 9*01 | CASSVEGSTEAF | 2207 |
| | | H5Y | | | | | | LIW | | | | F | 2428 |
| AE10 | Neo⁺WT⁻ | GNL3L- | 0 | 0 | 0 | 0 | | | | | 4-2*01 | CASSQAGGYEQY | 2429 |
| | | R4C | | | | | | | | | | F | |
| AE11 | Neo⁺WT⁻ | TEAD1- | 0 | 0 | 0 | 0 | 14/DV4*01 | CAMRANSGGYQK | | | 5-5*01 | CASTQPVDMNTE | 2208 |
| | | L9F | | | | | | VTF | | | | AFF | 2430 |
| AE12 | Neo⁺WT⁻ | SMARCD3- | 0 | 0 | 0 | 0 | | | | | 28*01 | CASSLYRGGDTQ | 2431 |
| | | H8Y | | | | | | | | | | YF | |
| AE2 | Neo⁺WT⁻ | GNL3L- | 0 | 0 | 0 | 0 | 6*01 | CALQTGANNLFF | | | 27*01 | CASSLWAGETQY | 2164 |
| | | R4C | | | | | | | | | | F | 2384 |
| AE3 | Neo⁺WT⁻ | GNL3L- | 0 | 0 | 0 | 0 | 6*01 | CALQTGANNLFF | | | 27*01 | CASSLWAGETQY | 2164 |
| | | R4C | | | | | | | | | | F | 2384 |
| AE4 | Neo⁺WT⁻ | MLL2-L8H | 0 | 0 | 0 | 0 | 12-2*01 | CAVGGYNFNKFYF | 12- | CVALRGGSQG | 5-6*01 | CASSFRDSSYEQ | 2204 |
| | | | | | | | | | 1*01 | NLIF | | YF | 2300 |
| | | | | | | | | | | | | | 2425 |
| AE5 | Neo⁺WT⁻ | FNDC3B- | 0 | 0 | 0 | 0 | 3*01 | CAVRDRAGGYQK | 13- | CAEIGNTGGFK | 13*01 | CASSSRLSQETQ | 2209 |
| | | L3M | | | | | | VTF | 2*01 | TIF | | YF | 2301 |
| | | | | | | | | | | | | | 2432 |
| AE6 | Neo⁺WT⁻ | PGM5- | 0 | 0 | 0 | 0 | 10*01 | CVVSLDYIPTF | | | 9*01 | CASSVEGSGETQ | 2210 |
| | | H5Y | | | | | | | | | | YF | 2433 |
| AE7 | Neo⁺WT⁻ | GNL3L- | 0 | 0 | 0 | 0 | 5*01 | CAEKNTDKLIF | 12- | CAVNRDDYKLS | 9*01 | CASSVSQGGYEQ | 2211 |
| | | R4C | | | | | | | 2*01 | F | | YF | 2302 |
| | | | | | | | | | | | | | 2434 |
| AE8 | Neo⁺WT⁻ | GNL3L- | 0 | 0 | 0 | 0 | 22*01 | CAVRTSYDKVIF | | | 20-1*01 | CSAPGGSGANVL | 2182 |
| | | R4C | | | | | | | | | | TF | 2435 |
| AE9 | Neo⁺WT⁻ | GANAB- | 0 | 0 | 0 | 0 | 8-3*01 | CAVAVWGNNAGN | 12- | CAVLTDSWGKL | 6-5*01 | CASSNVLAGGRD | 2212 |
| | | S5F | | | | | | MLTF | 2*01 | QF | | TQYF | 2303 |
| | | | | | | | | | | | | | 2436 |
| AF1 | Neo⁺WT⁻ | PGM5- | 0 | 0 | 0 | 0 | 8-2*01 | CVVSNSGNTPLVF | | | 7-9*01 | CASSLGDRGPQP | 2213 |
| | | H5Y | | | | | | | | | | QHF | 2437 |
| AF10 | Neo⁺WT⁻ | GNL3L- | 0 | 0 | 0 | 0 | 19*01 | CALSEANDGQKLL | | | 6-2*01, | CASTLAGGPYEQ | 2214 |
| | | R4C | | | | | | F | | | 6-3*01 | YF | 2438 |
| AF11 | Neo⁺WT⁻ | GANAB- | 0 | 0 | 0 | 0 | 1-1*01 | CAVSLYNQGGKLI | | | 19*01 | CASTGTDSYEQY | 2215 |
| | | S5F | | | | | | F | | | | F | 2439 |
| AF12 | Neo⁺WT⁻ | GNL3L- | 0 | 0 | 0 | 0 | 22*01 | CAVETSYDKVIF | | | | | 2216 |
| | | R4C | | | | | | | | | | | |
| AF2 | Neo⁺WT⁻ | GANAB- | 0 | 0 | 0 | 0 | 14/DV4*01 | CAMREPSQGGSE | | | 27*01 | CASSNQETQYF | 2217 |
| | | S5F | | | | | | KLVF | | | | | 2440 |
| AF3 | Neo⁺WT⁻ | GNL3L- | 0 | 0 | 0 | 0 | 38- | CASAGTSYDKVIF | | | 20-1*01 | CSVRTPSSYEQY | 2218 |
| | | R4C | | | | | 2/DV8*01 | | | | | F | 2441 |
| AF4 | Neo⁺WT⁻ | GNL3L- | 0 | 0 | 0 | 0 | 13-2*01 | CAESSSGSARQLT | | | 4-3*01 | CASSQVPGGYEQ | 2219 |
| | | R4C | | | | | | F | | | | YF | 2442 |
| AF5 | Neo⁺WT⁻ | GANAB- | 0 | 0 | 0 | 0 | 29/DV5*01 | CAASAQGGTSYG | | | 19*01 | CASRMGTSGSTD | 2220 |
| | | S5F | | | | | | KLTF | | | | TQYF | 2443 |
| AF6 | Neo⁺WT⁻ | GNL3L- | 0 | 0 | 0 | 0 | | | | | 20-1*01 | CSALGLAGGQGG | 2444 |
| | | R4C | | | | | | | | | | ELFF | |
| AF7 | Neo⁺WT⁻ | GNL3L- | 0 | 0 | 0 | 0 | 22*01 | CAVKTSYDKVIF | | | 20-1*01 | CSAGVYEQYF | 2199 |
| | | R4C | | | | | | | | | | | 2445 |
| AF8 | Neo⁺WT⁻ | GNL3L- | 0 | 0 | 0 | 0 | 12-2*01 | CAVFYGNNRLAF | | | 9*01 | CASSVWDSLTGE | 2221 |
| | | R4C | | | | | | | | | | LFF | 2446 |
| AF9 | Neo⁺WT⁻ | GNL3L- | 0 | 0 | 0 | 0 | 22*01 | CAVRTSYDKVIF | | | 19*01 | CASSWDNGGYT | 2182 |
| | | R4C | | | | | | | | | | F | 2447 |
| CA1 | Neo⁺WT⁻ | NSDHL- | 0 | 0 | 0 | 0 | 19*01 | CALSEVITGANNLF | | | 9*01 | CASSVGSQETQY | 2222 |
| | | A9V | | | | | | F | | | | F | 2448 |
| CA10 | Neo⁺WT⁻ | PGM5- | 0 | 0 | 0 | 0 | 1-1*01 | CAVRDWYGGSQG | | | 6-1*01 | CASILGLTTYNEQ | 2223 |
| | | H5Y | | | | | | NLIF | | | | FF | 2449 |
| CA11 | Neo⁺WT⁻ | GNL3L- | 0 | 0 | 0 | 0 | 29/DV5*01 | CAGADKLIF | 27*01 | CAGDGGSQGN | 6-5*01 | CASSWTGAGYE | 2224 |
| | | R4C | | | | | | | | LIF | | QYF | 2304 |
| | | | | | | | | | | | | | 2450 |
| CA12 | Neo⁺WT⁻ | 0 | 0 | 0 | 0 | 0 | 5*01 | CAESSFYVSGGYN | | | 11-3*01 | CASSLGETQYF | 2225 |
| | | | | | | | | KLIF | | | | | 2451 |
| CA2 | Neo⁺WT⁻ | GNL3L- | 0 | 0 | 0 | 0 | 8-2*01 | CVVSDKEWGGGA | | | 14*01 | | 2226 |
| | | R4C | | | | | | DGLTF | | | | | |
| CA3 | Neo⁺WT⁻ | 0 | 0 | 0 | 0 | 0 | | | | | 2*01 | CASRYREGVEKL | 2452 |
| | | | | | | | | | | | | FF | |
| CA4 | Neo⁺WT⁻ | 0 | 0 | 0 | 0 | 0 | | | | | | | |
| CA7 | Neo⁺WT⁻ | 0 | 0 | 0 | 0 | 0 | 13-1*01 | CAAPRNDKIIF | | | 6-5*01 | CASSYSGPTGYE | 2227 |
| | | | | | | | | | | | | QYF | 2453 |
| CA8 | Neo⁺WT⁻ | GANAB- | 0 | 0 | 0 | 0 | 7*01 | CALGELVTGGGNK | | | 5-6*01 | CASSLNREGNTE | 2228 |
| | | S5F | | | | | | LTF | | | | AFF | 2454 |
| CA9 | Neo⁺WT⁻ | MLL2-L8H | 0 | 0 | 0 | 0 | 12-2*01 | CAVISNQFYF | | | 6-5*01 | CASSYEGALSYE | 2229 |
| | | | | | | | | | | | | QYF | 2455 |
| CB1 | Neo⁺WT⁻ | GNL3L- | 0 | 0 | 0 | 0 | 25*01 F | PNYGGSQGNLIF | | | 20-1*01 | CSAREGLAAGEL | 2230 |
| | | R4C | | | | | | | | | | FF | 2456 |
| CB10 | Neo⁺WT⁻ | GNL3L- | 0 | 0 | 0 | 0 | 12-2*01 | CAVNPRDDKIIF | | | 3-1*01 | CASSPGQGLAYE | 2231 |
| | | R4C | | | | | | | | | | QYF | 2457 |
| CB11 | Neo⁺WT⁻ | FNDC3B- | 0 | 0 | 0 | 0 | 12-2*01 | CAVKDRGGSEKLV | | | 6-6*01 | CASRDSLTGELF | 2232 |
| | | L3M | | | | | | F | | | | F | 2458 |
| CB12 | Neo⁺WT⁻ | GNL3L- | 0 | 0 | 0 | 0 | | | | | 6-5*01 | CASSPSGGSYGY | 2459 |
| | | R4C | | | | | | | | | | TF | |
| CB2 | Neo⁺WT⁻ | GNL3L- | 0 | 0 | 0 | 0 | 13-1*01 | CAASNDQKLVF | | | 9*01 | CASSISTSGYEQF | 2233 |
| | | R4C | | | | | | | | | | F | 2460 |
| CB3 | Neo⁺WT⁻ | GNL3L- | 0 | 0 | 0 | 0 | 13-1*01 | CAAFSNQAGTALIF | | | 6-5*01 | CASSYSNGGYGY | 2234 |
| | | R4C | | | | | | | | | | TF | 2461 |
| CB7 | Neo⁺WT⁻ | 0 | 0 | 0 | 0 | 0 | | | | | 7-2*01 | CASSFWTSGGE | 2462 |
| | | | | | | | | | | | | QYF | |
| CB8 | Neo⁺WT⁻ | 0 | 0 | 0 | 0 | 0 | 8-3*01 | CAVGFDNNAGNM | | | 10-3*01 | CAISERWDGYNE | 2235 |
| | | | | | | | | LTF | | | | QFF | 2463 |
| CC1 | Neo⁺WT⁻ | GNL3L- | 0 | 0 | 0 | 0 | | | | | 7-6*01 | CASSFLGDEQFF | 2464 |
| | | R4C | | | | | | | | | | | |
| CC10 | Neo⁺WT⁻ | NSDHL- | 0 | 0 | 0 | 0 | | | | | 15*01 | CATSRDLGGQQP | 2465 |
| | | A9V | | | | | | | | | | QHF | |
| CC12 | Neo⁺WT⁻ | GNL3L- | 0 | 0 | 0 | 0 | 22*01 | CAVYSSASKIIF | 10*01 | CVVNPYNTDKLI | 4-3*01 | CASSVGEGTEAF | 2197 |
| | | R4C | | | | | | | | F | | F | 2305 |
| | | | | | | | | | | | | | 2466 |
| CC2 | Neo⁺WT⁻ | USP28- | 0 | 0 | 0 | 0 | 30*01 | CGTRGGSGNTPL | 35*01 | CAGQMYSGGG | 12-3*01, | CASTATFGVTEA | 2236 |
| | | C5F | | | | | | VF | | ADGLTF | 12-4*01 | FF | 2306 |
| | | | | | | | | | | | | | 2467 |
| CC3 | Neo⁺WT⁻ | GNL3L- | 0 | 0 | 0 | 0 | 12-2*01 | CAVANDYKLSF | | | 6-5*01 | CASSYSLAAEAFF | 2237 |
| | | R4C | | | | | | | | | | | 2468 |
| CC4 | Neo⁺WT⁻ | MRM1- | 0 | 0 | 0 | 0 | | | | | 14*01 | CASSLTGSEQYF | 2469 |
| | | T6P | | | | | | | | | | | |
| CC7 | Neo⁺WT⁻ | 0 | 0 | 0 | 0 | 0 | 12-2*01 | CALLTEDSNYQLI | | | 2*01 | CASSGELGSPLH | 2238 |
| | | | | | | | | W | | | | F | 2470 |
| CD1 | Neo⁺WT⁻ | GANAB- | 0 | 0 | 0 | 0 | 8-1*01 | CAVIPDSNYQLIW | | | 5-8*01 | CASSSLGEQFF | 2239 |
| | | S5F | | | | | | | | | | | 2471 |
| CD10 | Neo⁺WT⁻ | AKAP13- | 0 | 0 | 0 | 0 | 38- | CAYYTPLVF | | | 6-2*01, | CASTDTGELFF | 2240 |
| | | Q8K | | | | | 2/DV8*01 | | | | 6-3*01 | | 2472 |
| CD11 | Neo⁺WT⁻ | GNL3L- | 0 | 0 | 0 | 0 | 22*01 | CAVRTSYDKVIF | | | 29-1*01 | CSVEGPGGRIAN | 2182 |
| | | R4C | | | | | | | | | | TEAFF | 2473 |
| CD2 | Neo⁺WT⁻ | GNL3L- | 0 | 0 | 0 | 0 | | | | | 27*01 | CASSLWAGETQY | 2384 |
| | | R4C | | | | | | | | | | F | |
| CD4 | Neo⁺WT⁻ | NSDHL- | 0 | 0 | 0 | 0 | | | | | 5-5*01 | CASSARGYDEQF | 2474 |
| | | A9V | | | | | | | | | | F | |
| CD5 | Neo⁺WT⁻ | GNL3L- | 0 | 0 | 0 | 0 | 22*01 | CAVDPNTGNQFYF | | | 20-1*01 | CSARASGAYEQY | 2241 |
| | | R4C | | | | | | | | | | F | 2475 |
| CD7 | Neo⁺WT⁻ | 0 | 0 | 0 | 0 | 0 | 12-2*01 | CAVNTGNQFYF | | | 6-5*01 | CASSYANGYEQY | 2242 |
| | | | | | | | | | | | | F | 2476 |
| CD8 | Neo⁺WT⁻ | GNL3L- | 0 | 0 | 0 | 0 | | | | | | | |
| | | R4C | | | | | | | | | | | |
| CD9 | Neo⁺WT⁻ | USP28- | 0 | 0 | 0 | 0 | 30*01 | CGTRGGSGNTPL | 35*01 | CAGQMYSGGG | 12-3*01, | CASTATFGVTEA | 2236 |
| | | CSF | | | | | | VF | | ADGLTF | 12-4*01 | FF | 2306 |
| | | | | | | | | | | | | | 2467 |
| CE1 | Neo⁺WT⁻ | 0 | 0 | 0 | 0 | 0 | | | | | 7-7*01 | CASSWGGGYEQ | 2477 |
| | | | | | | | | | | | | YF | |
| CE10 | Neo⁺WT⁻ | GNL3L- | 0 | 0 | 0 | 0 | 12-2*01 | CAVLLYGNKLVF | | | 6-1*01 | CASNQGLYEQYF | 2243 |
| | | R4C | | | | | | | | | | | 2478 |
| CE11 | Neo⁺WT⁻ | TEAD1- | 0 | 0 | 0 | 0 | 38- | CALTQGGSEKLVF | | | 19*01 | CASSIAQGGNQP | 2244 |
| | | L8F | | | | | 2/DV8*01 | | | | | QHF | 2479 |
| CE12 | Neo⁺WT⁻ | GNL3L- | 0 | 0 | 0 | 0 | | | | | | | |
| | | R4C | | | | | | | | | | | |
| CE2 | Neo⁺WT⁻ | GANAB- | 0 | 0 | 0 | 0 | 12-2*01 | CAVTTDSWGKLQF | | | 6-2*01, | CASSRQPMNTEA | 2245 |
| | | S5F | | | | | | | | | 6-3*01 | FF | 2480 |
| CE3 | Neo⁺WT⁻ | MLL2-L8H | 0 | 0 | 0 | 0 | | | | | 6-2*01, | CASSYSLEGYTF | 2481 |
| | | | | | | | | | | | 6-3*01 | | |
| CE4 | Neo⁺WT⁻ | SEC24A- | 0 | 0 | 0 | 0 | 26-1*01 | CIVRVDNARLMF | 26- | CIVRVRDSNYQ | 7-9*01 | | 2246 |
| | | PSL | | | | | | | 1*01 | LIW | | | 2307 |
| CE5 | Neo⁺WT⁻ | GNL3L- | 0 | 0 | 0 | 0 | 22*01 | CAVKTSYDKVIF | | | 20-1*01 | CSARVTSGSYEQ | 2199 |
| | | R4C | | | | | | | | | | YF | 2482 |
| CE6 | Neo⁺WT⁻ | 0 | 0 | 0 | 0 | 0 | | | | | | | |
| CE7 | Neo⁺WT⁻ | GANAB- | 0 | 0 | 0 | 0 | 14/DV4*01 | CAMREDAGGTSY | | | 29-1*01 | CSVGTYSNQPQH | 2247 |
| | | S5F | | | | | | GKLTF | | | | F | 2483 |
| CE9 | Neo⁺WT⁻ | GNL3L- | 0 | 0 | 0 | 0 | 12-2*01 | CAVGNSGGYQKV | | | 6-1*01 | CASSEGGYTEAF | 2248 |
| | | R4C | | | | | | TF | | | | F | 2484 |
| CF1 | Neo⁺WT⁻ | GNL3L- | 0 | 0 | 0 | 0 | | | | | 3-1*01 | CASSPGDGTEAF | 2485 |
| | | R4C | | | | | | | | | | F | |
| CF11 | Neo⁺WT⁻ | MRM1- | 0 | 0 | 0 | 0 | 24*01 | CAFSDGQKLLF | | | 7-9*01 | CASSLPPADMRD | 2249 |
| | | T6P | | | | | | | | | | TQYF | 2486 |
| CF12 | Neo⁺WT⁻ | GNL3L- | 0 | 0 | 0 | 0 | 22*01 | CAVKTSYDKVIF | | | 20-1*01 | CSSVTEAFF | 2199 |
| | | R4C | | | | | | | | | | | 2487 |
| CF2 | Neo⁺WT⁻ | WDR46- | 0 | 0 | 0 | 0 | 8-1*01 | CAVKMDSNYQLIW | | | 4-2*01 | CASSQDRGNEQF | 2250 |
| | | T3I | | | | | | | | | | F | 2488 |
| CF3 | Neo⁺WT⁻ | PGM5- | 0 | 0 | 0 | 0 | | | | | 20-1*01 | | |
| | | H5Y | | | | | | | | | | | |
| CF4 | Neo⁺WT⁻ | PABPC1- | 0 | 0 | 0 | 0 | | | | | 7-9*01 | CASSFGSGEQFF | 2489 |
| | | R5Q | | | | | | | | | | | |
| CF5 | Neo⁺WT⁻ | GANAB- | 0 | 0 | 0 | 0 | 12-2*01 | CAVTGSGYALNF | | | 4-3*01 | CASSQAHTGELF | 2251 |
| | | S5F | | | | | | | | | | F | 2490 |
| CF6 | Neo⁺WT⁻ | 0 | 0 | 0 | 0 | 0 | | | | | | | |
| CF7 | Neo⁺WT⁻ | GNL3L- | 0 | 0 | 0 | 0 | | | | | 4-3*01 | CASSQDRDSGYY | 2491 |
| | | R4C | | | | | | | | | | EQYF | |
| CF8 | Neo⁺WT⁻ | GNL3L- | 0 | 0 | 0 | 0 | 13-1*01 | CAATSNTGKLIF | 13- | CAAFSHTNAGK | 6-5*01 | CASSYSSGYFLF | 2252 |
| | | R4C | | | | | | | 1*01 | STF | | F | 2308 |
| | | | | | | | | | | | | | 2492 |
| CF9 | Neo⁺WT⁻ | GNL3L- | 0 | 0 | 0 | 0 | 12-1*01 | CVGMDSSYKLIF | | | 6-5*01 | CASSPSTGYGYT | 2253 |
| | | R4C | | | | | | | | | | F | 2493 |
| CG1 | Neo⁺WT⁻ | GANAB- | 0 | 0 | 0 | 0 | | | | | 19*01 | CASSTGNYGYTF | 2494 |
| | | S5F | | | | | | | | | | | |
| CG10 | Neo⁺WT⁻ | GNL3L- | 0 | 0 | 0 | 0 | | | | | | | |
| | | R4C | | | | | | | | | | | |
| CG11 | Neo⁺WT⁻ | GNL3L- | 0 | 0 | 0 | 0 | | | | | | | |
| | | R4C | | | | | | | | | | | |
| CG12 | Neo⁺WT⁻ | MRM1- | 0 | 0 | 0 | 0 | 21*01 | CAVRYYFGNEKLT | | | 5-1*01 | CASSLIQGAVDT | 2254 |
| | | T6P | | | | | | F | | | | QYF | 2495 |
| CG2 | Neo⁺WT⁻ | GNL3L- | 0 | 0 | 0 | 0 | 6*01 | CALQTGANNLFF | 14/DV | CAMIFNDYKLSF | 27*01 | CASSLWAGETQY | 2164 |
| | | R4C | | | | | | | 4*01 | | | F | 2261 |
| | | | | | | | | | | | | | 2384 |
| CG3 | Neo⁺WT⁻ | GNL3L- | 0 | 0 | 0 | 0 | | | | | 6-5*01 | CASSFGQGYEQY | 2496 |
| | | R4C | | | | | | | | | | F | |
| CG4 | Neo⁺WT⁻ | GANAB- | 0 | 0 | 0 | 0 | 21*01 | CAASGGGADGLTF | | | 6-5*01 | CASSPWTLNEQY | 2255 |
| | | S5F | | | | | | | | | | F | 2497 |
| CG5 | Neo⁺WT⁻ | PABPC1- | 0 | 0 | 0 | 0 | 12-2*01 | CAVIPRGGSNYKL | | | 6-2*01, | CASSYGNTGELF | 2256 |
| | | R5Q | | | | | | TF | | | 6-3*01 | F | 2498 |
| CG7 | Neo⁺WT⁻ | GNL3L- | 0 | 0 | 0 | 0 | 13-1*01 | CAYGGGTYKYIF | | | 6-2*01, | CASSYSDRSSYE | 2257 |
| | | R4C | | | | | | | | | 6-3*01 | QYF | 2499 |
| CG8 | Neo⁺WT⁻ | GNL3L- | 0 | 0 | 0 | 0 | 12-2*01 | CAVMTGGFKTIF | | | 6-5*01 | CASSYGGGYEQY | 2258 |
| | | R4C | | | | | | | | | | F | 2500 |
| CG9 | Neo⁺WT⁻ | PGM5- | 0 | 0 | 0 | 0 | | | | | | | |
| | | H5Y | | | | | | | | | | | |
| CH12 | Neo⁺WT⁻ | USP28- | 0 | 0 | 0 | 0 | 19*01 | CALTQSGGYQKVT | | | 2*01 | CASREGLEDTEA | 2259 |
| | | C5F | | | | | | F | | | | FF | 2501 |
| CH7 | Neo⁺WT⁻ | PGM5- | 0 | 0 | 0 | 0 | 19*01 | CALGDYKLSF | | | 13*01 | CASTEGQGGEQ | 2260 |
| | | H5Y | | | | | | | | | | YF | 2502 |
| CH9 | Neo⁺WT⁻ | GNL3L- | 0 | 0 | 0 | 0 | | | | | 29-1*01 | CSPGDGYTF | 2503 |
| | | R4C | | | | | | | | | | | |
| AG1 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG10 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG11 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG12 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG2 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG3 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG4 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG5 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG6 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG7 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG8 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| AG9 | Spike-In | HCV-KLV | 0 | 0 | 0 | 0 | 14/DV4*01 | CAMIFNDYKLSF | | | 19*01 | CASSTGNYGYTF | 2261 |
| | Clone | | | | | | | | | | | | 2494 |
| BA12 | Neo⁻WT⁺ | ERBB2 | 0 | 0 | 0 | 0 | 8-4*01 | CAVSDLNSGGYQ | | | 18*01 | CASSPRDRVHEQ | 2262 |
| | | | | | | | | KVTF | | | | YF | 2504 |
| BA4 | Neo⁻WT⁺ | GANAB | 0 | 0 | 0 | 0 | 12-2*01 | CAVNNARLMF | | | 4-3*01 | CASSQGGGGTD | 2263 |
| | | | | | | | | | | | | TQYF | 2505 |
| BA5 | Neo⁻WT⁺ | MRM1 | 0 | 0 | 0 | 0 | 12-2*01 | CAVNNARLMF | | | 4-1*01 | CASSPSPGSEQY | 2263 |
| | | | | | | | | | | | | F | 2506 |
| BA7 | Neo⁻WT⁺ | GANAB | 0 | 0 | 0 | 0 | 12-2*01 | CAIEGGKLIF | | | 2*01 | CASSDWGGETQ | 2264 |
| | | | | | | | | | | | | YF | 2507 |
| BB2 | Neo⁻WT⁺ | GANAB | 0 | 0 | 0 | 0 | 12-2*01 | CAVNNARLMF | | | 4-3*01 | CASSQGGGGTD | 2263 |
| | | | | | | | | | | | | TQYF | 2505 |
| BB3 | Neo⁻WT⁺ | GANAB | 0 | 0 | 0 | 0 | 12-3*01 | CAMKDFGNEKLTF | | | 2*01 | CSWDFQETQYF | 2265 |
| | | | | | | | | | | | | | 2508 |
| BB4 | Neo⁻WT⁺ | TEAD1- | 0 | 0 | 0 | 0 | 12-2*01 | CAVITGTALIF | | | 2*01 | CASSENTGELFF | 2266 |
| | | (SVL) | | | | | | | | | | | 2509 |
| BB5 | Neo⁻WT⁺ | GANAB | 0 | 0 | 0 | 0 | 12-2*01 | CAVNNARLMF | | | | | 2263 |
| BB9 | Neo⁻WT⁺ | FNDC3B | 0 | 0 | 0 | 0 | | | | | | | |
| BC3 | Neo⁻WT⁺ | FNDC3B | 0 | 0 | 0 | 0 | 14/DV4*01 | CAMREFNAGGTS | | | 20-1*01 | CSGLVPGFDSPL | 2267 |
| | | | | | | | | YGKLTF | | | | HF | 2510 |
| BC6 | Neo⁻WT⁺ | FNDC3B | 0 | 0 | 0 | 0 | 14/DV4*01 | CAMRETWGGLGG | 12- | CVVISTDSWGK | 9*01 | CASSVETGGLDT | 2268 |
| | | | | | | | | SQGNLIF | 1*01 | FQF | | QYF | 2309 |
| | | | | | | | | | | | | | 2375 |
| BC7 | Neo⁻WT⁺ | PGM5 | 0 | 0 | 0 | 0 | | | | | 9*01 | CASSVDGGPQET | 2511 |
| | | | | | | | | | | | | QYF | |
| BD4 | Neo⁻WT⁺ | FNDC3B | 0 | 0 | 0 | 0 | 12-2*01 | CAVYTGGFKTIF | | | 12-3*01, | CASSFGGSSYEQ | 2269 |
| | | | | | | | | | | | 12-4*01 | YF | 2512 |
| BD6 | Neo⁻WT⁺ | WDR46 | 0 | 0 | 0 | 0 | 12-2*01 | CAVPVLGGSQGNL | | | | | 2270 |
| | | | | | | | | IF | | | | | |
| BD7 | Neo⁻WT⁺ | SEC24A | 0 | 0 | 0 | 0 | | | | | 9*01 | CASSVGTSSYGY | 2513 |
| | | | | | | | | | | | | TF | |
| BD9 | Neo⁻WT⁺ | MLL2 | 0 | 0 | 0 | 0 | 17*01 | CATDANTGNQFYF | | | 19*01 | CASSLGTLNEQF | 2271 |
| | | | | | | | | | | | | F | 2514 |
| BE11 | Neo⁻WT⁺ | SEC24A | 0 | 0 | 0 | 0 | 22*01 | CALLSNQAGTALIF | | | 28*01 | CASSNARGYGYT | 2272 |
| | | | | | | | | | | | | F | 2515 |
| BE2 | Neo⁻WT⁺ | USP28 | 0 | 0 | 0 | 0 | 8-3*01 | CAVGDAGGATNKL | | | 7-2*01 | CASSWWLNTEAF | 2273 |
| | | | | | | | | IF | | | | F | 2516 |
| BE4 | Neo⁻WT⁺ | GANAB | 0 | 0 | 0 | 0 | 12-2*01 | CAVNNARLMF | | | | | 2263 |
| BE6 | Neo⁻WT⁺ | SEC24A | 0 | 0 | 0 | 0 | | | | | 5-6*01 | CASSPAGSNYGY | 2517 |
| | | | | | | | | | | | | TF | |
| BE7 | Neo⁻WT⁺ | MRM1 | 0 | 0 | 0 | 0 | 9-2*01 | CALSEPIYNFNKFY | | | 11-2*01 | CASSLGAEQYF | 2274 |
| | | | | | | | | F | | | | | 2518 |
| BE8 | Neo⁻WT⁺ | SEC24A | 0 | 0 | 0 | 0 | 22*01 F | CAVEDLGFGNVLH | | | 28*01 | CASSPGLYTQYF | 2275 |
| | | | | | | | | C | | | | | 2519 |
| BF1 | Neo⁻WT⁺ | SEC24A | 0 | 0 | 0 | 0 | | | | | | | |
| BF10 | Neo⁻WT⁺ | GANAB | 0 | 0 | 0 | 0 | 12-2*01 | CAVNPGGFKTIF | | | 6-2*01, | CASSYSSGTEAF | 2276 |
| | | | | | | | | | | | 6-3*01 | F | 2520 |
| BF2 | Neo⁻WT⁺ | GANAB | 0 | 0 | 0 | 0 | 12-2*01 | CAVSPGGFKTIF | | | | | 2277 |
| BF4 | Neo⁻WT⁺ | SEC24A | 0 | 0 | 0 | 0 | 5*01 | CAERDQAGTALIF | 17*01 | CATDVYDYKLS | 7-2*01 | CASSLREAGELF | 2278 |
| | | | | | | | | | | F | | F | 2310 |
| | | | | | | | | | | | | | 2521 |
| BF6 | Neo⁻WT⁺ | SEC24A | 0 | 0 | 0 | 0 | 8-3*01 | CAVGYNTDKLIF | | | 7-6*01 | CASSLGNTEAFF | 2279 |
| | | | | | | | | | | | | | 2522 |
| BF7 | Neo⁻WT⁺ | FNDC3B | 0 | 0 | 0 | 0 | 1-2*01 | CAVRGSARQLTF | | | 2*01 | CASSEVQGGRDT | 2280 |
| | | | | | | | | | | | | QYF | 2523 |
| BF8 | Neo⁻WT⁺ | SEC24A | 0 | 0 | 0 | 0 | 12-3*01 | CAMDKMDSNYQLI | | | 27*01 | CASSFGIGPQYF | 2281 |
| | | | | | | | | W | | | | | 2524 |
| BG3 | Neo⁻WT⁺ | WDR46 | 0 | 0 | 0 | 0 | 14/DV4*01 | CAMRESKAAGNKL | | | 7-2*01 | CASSLWGQGWT | 2282 |
| | | | | | | | | TF | | | | GELFF | 2525 |
| BG4 | Neo⁻WT⁺ | COL18A1 | 0 | 0 | 0 | 0 | 23/DV6*01 | CAASLNTNAGKST | | | 30*01 | CAWSVGNYGYTF | 2283 |
| | | | | | | | | F | | | | | 2526 |
| BG6 | Neo⁻WT⁺ | FNDC3B | 0 | 0 | 0 | 0 | 14/DV4*01 | CAMRESSYGNNR | | | 5-8*01 | CASSRPLNQPQH | 2284 |
| | | | | | | | | LAF | | | | F | 2527 |
| BG8 | Neo⁻WT⁺ | WDR46 | 0 | 0 | 0 | 0 | 12-2*01 | CAVNMEGAGSYQ | | | 9*01 | CASSVESGEQYF | 2285 |
| | | | | | | | | LTF | | | | | 2528 |
| BH12 | Neo⁻WT⁺ | GANAB | 0 | 0 | 0 | 0 | 12-2*01 | CAVNNARLMF | | | | | 2263 |
| BH3 | Neo⁻WT⁺ | SNX24 | 0 | 0 | 0 | 0 | 13-2*01 | CAENKDDYKLSF | | | 7-8*01 | CASSFSATGELFF | 2286 |
| | | | | | | | | | | | | | 2529 |
| BH4 | Neo⁻WT⁺ | GANAB | 0 | 0 | 0 | 0 | 12-2*01 | CAVNNARLMF | | | | | 2263 |
| CB9 | Neo⁻WT⁺ | PGM5 | 0 | 0 | 0 | 0 | 35*01 | CAGAEISGGGADG | 9-2*01 | CAPPIEGGSEKL | 3-1*01 | CASSLAYEQYF | 2287 |
| | | | | | | | | LTF | | VF | | | 2311 |
| | | | | | | | | | | | | | 2530 |
| CC5 | Neo-WT⁺ | WDR46 | 0 | 0 | 0 | 0 | | | | | 4-1*01 | CASSFGANTGEL | 2531 |
| | | | | | | | | | | | | FF | |
| CC8 | Neo⁻WT⁺ | SEC24A | 0 | 0 | 0 | 0 | | | | | | | |
| CD6 | Neo⁻WT⁺ | GANAB | 0 | 0 | 0 | 0 | 12-2*01 | CAVNNARLMF | | | 4-3*01 | CASSQGGGGTD | 2563 |
| | | | | | | | | | | | | TQYF | 2505 |
| CH11 | Neo⁻WT⁺ | GANAB | 0 | 0 | 0 | 0 | 12-2*01 | CAVNNARLMF | | | 4-3*01 | CASSQGGGGTD | 2263 |
| | | | | | | | | | | | | TQYF | 2505 |
| CH4 | Neo⁻WT⁺ | SEC24A | 0 | 0 | 0 | 0 | 14/DV4*01 | CAMREFYSGGGA | | | 2*01 | CASSEDRGNSPL | 2288 |
| | | | | | | | | DGLTF | | | | HF | 2532 |
| CH5 | Neo⁻WT⁺ | GANAB | 0 | 0 | 0 | 0 | 12-2*01 | CAVNNARLMF | | | 4-3*01 | CASSQGGGGTD | 2263 |
| | | | | | | | | | | | | TQYF | 2505 |

TABLE 7
TetTCR summary for experiment 4.

| Cell | Sorted | Detected Peptide by MID Count | | | | | TCRα, 1 | | TCRα, 2 | | TCRβ | | SEQ ID NO |
|---|---|---|---|---|---|---|---|---|---|---|---|---|---|
| Name | Population | Rank 1 | Rank 2 | Rank 3 | Rank 4 | Rank 5 | TRAV | CDR3α | TRAV | CDR3α | TRBV | CDR3β | (L to R) |
| G23 | Neo⁺WT⁺ | 0 | 0 | 0 | 0 | 0 | | | | | | | |
| G6 | Neo⁺WT⁺ | 0 | 0 | 0 | 0 | 0 | | | | | 15*01 | CATSQMGDT | 2615 |
| | | | | | | | | | | | | QYF | |
| H10 | Neo⁺WT⁺ | 0 | 0 | 0 | 0 | 0 | 3*01 | CAVGFYGNN | | | | | 2533 |
| | | | | | | | | RLAF | | | | | |
| H9 | Neo⁺WT⁺ | 0 | 0 | 0 | 0 | 0 | | | | | 6-2*01, | CASSPFGDM | 2616 |
| | | | | | | | | | | | 6-3*01 | LYNEQFF | |
| I12 | Neo⁺WT⁺ | 0 | 0 | 0 | 0 | 0 | 12-2*01 | CAVRNNDMR | | | | | 2534 |
| | | | | | | | | F | | | | | |
| I15 | Neo⁺WT⁺ | 0 | 0 | 0 | 0 | 0 | | | | | 30*01 | CAARPASYE | 2617 |
| | | | | | | | | | | | | QYF | |
| J5 | Neo⁺WT⁺ | 0 | 0 | 0 | 0 | 0 | | | | | 12-3*01, | CASSSSGRA | 2618 |
| | | | | | | | | | | | 12-4*01 | SADTQYF | |
| K5 | Neo⁺WT⁺ | 0 | 0 | 0 | 0 | 0 | | | | | | | |
| L13 | Neo⁺WT⁺ | 0 | 0 | 0 | 0 | 0 | | | | | | | |
| L6 | Neo⁺WT⁺ | 0 | 0 | 0 | 0 | 0 | 19*01 | CALSEALAY | | | | | 2535 |
| | | | | | | | | NQGGKLIF | | | | | |
| M3 | Neo⁺WT⁺ | 0 | 0 | 0 | 0 | 0 | 10*01 | CVVSGGYNK | | | 4-2*01 | CASSPNARLA | 2536 2619 |
| | | | | | | | | LIF | | | | GAGGTDTQYF | |
| M7 | Neo⁺WT⁺ | 0 | 0 | 0 | 0 | 0 | | | | | 5-6*01 | CASSLAPKT | 2620 |
| | | | | | | | | | | | | AFSYEQYF | |
| O1 | Neo⁺WT⁺ | 0 | 0 | 0 | 0 | 0 | 14/DV4*01 | CAMRDPFTG | | | 20-1*01 | CSARSWTPQ | 2537 2621 |
| | | | | | | | | NQFYF | | | | ETQYF | |
| H23 | Neo⁺WT⁺ | AKAP13 | 0 | 0 | 0 | 0 | | | | | | | |
| H4 | Neo⁺WT⁺ | AKAP13 | AKAP13_Q8K | 0 | 0 | 0 | | | | | | | |
| K2 | Neo⁺WT⁺ | AKAP13 | AKAP13_Q8K | SNX24 | 0 | 0 | | | | | 29-1*01 | CSVEGLRGG | 2622 |
| | | | | | | | | | | | | NEQFF | |
| G2 | Neo⁺WT⁺ | AKAP13_Q8K | AKAP13 | 0 | 0 | 0 | | | | | | | |
| I1 | Neo⁺WT⁺ | COL18A1 | COL18A1_S8F | 0 | 0 | 0 | 16*01 | CALRGYSTL | | | | | 2538 |
| | | | | | | | | TF | | | | | |
| K12 | Neo⁺WT⁺ | COL18A1 | COL18A1_S8F | 0 | 0 | 0 | | | | | | | |
| G12 | Neo⁺WT⁺ | FNDC3B | FNDC3B_L3M | 0 | 0 | 0 | | | | | | | |
| G14 | Neo⁺WT⁺ | FNDC3B | FNDC3B_L3M | 0 | 0 | 0 | 14/DV4*01 | CAMRELGGS | | | | | 2539 |
| | | | | | | | | NYKLTF | | | | | |
| G18 | Neo⁺WT⁺ | FNDC3B | FNDC3B_L3M | 0 | 0 | 0 | | | | | 13*01 | CASSLGGLT | 2623 |
| | | | | | | | | | | | | DTQYF | |
| G19 | Neo⁺WT⁺ | FNDC3B | FNDC3B_L3M | 0 | 0 | 0 | | | | | | | |
| G24 | Neo⁺WT⁺ | FNDC3B | FNDC3B_L3M | 0 | 0 | 0 | | | | | | | |
| G3 | Neo⁺WT⁺ | FNDC3B | 0 | 0 | 0 | 0 | | | | | 30*01 | CAWSAGEQY | 2624 |
| | | | | | | | | | | | | F | |
| G7 | Neo⁺WT⁺ | FNDC3B | USP28_C5F | 0 | 0 | 0 | 19*01 | CALSETDTG | | | | | 2540 |
| | | | | | | | | RRALTF | | | | | |
| H11 | Neo⁺WT⁺ | FNDC3B | FNDC3B_L3M | 0 | 0 | 0 | | | | | | | |
| H2 | Neo⁺WT⁺ | FNDC3B | FNDC3B_L3M | 0 | 0 | 0 | | | | | | | |
| H8 | Neo⁺WT⁺ | FNDC3B | FNDC3B_L3M | 0 | 0 | 0 | | | | | | | |
| I13 | Neo⁺WT⁺ | FNDC3B | FNDC3B_L3M | 0 | 0 | 0 | | | | | | | |
| I14 | Neo⁺WT⁺ | FNDC3B | FNDC3B_L3M | 0 | 0 | 0 | 14/DV4*01 | CAMREFAGA | | | | | 2541 |
| | | | | | | | | NSKLTF | | | | | |
| I18 | Neo⁺WT⁺ | FNDC3B | FNDC3B_L3M | 0 | 0 | 0 | | | | | 6-5*01 | CASSYGGGS | 2625 |
| | | | | | | | | | | | | PQYF | |
| I20 | Neo⁺WT⁺ | FNDC3B | FNDC3B_L3M | 0 | 0 | 0 | | | | | | | |
| I6 | Neo⁺WT⁺ | FNDC3B | FNDC3B_L3M | 0 | 0 | 0 | 12-2*01 | CAVNNARLM | | | | | 2542 |
| | | | | | | | | F | | | | | |
| J1 | Neo⁺WT⁺ | FNDC3B | FNDC3B_L3M | 0 | 0 | 0 | | | | | 5-4*01 | CASSWTGN | 2626 |
| | | | | | | | | | | | | TEAFF | |
| J12 | Neo⁺WT⁺ | FNDC3B | FNDC3B_L3M | 0 | 0 | 0 | | | | | | | |
| J17 | Neo⁺WT⁺ | FNDC3B | FNDC3B_L3M | 0 | 0 | 0 | | | | | | | |
| K19 | Neo⁺WT⁺ | FNDC3B | FNDC3B_L3M | 0 | 0 | 0 | | | | | | | |
| K3 | Neo⁺WT⁺ | FNDC3B | FNDC3B_L3M | 0 | 0 | 0 | | | | | | | |
| L1 | Neo⁺WT⁺ | FNDC3B | FNDC3B_L3M | 0 | 0 | 0 | 29/DV5*01 | CAASGQGGT | | | | | 2543 |
| | | | | | | | | SYGKLTF | | | | | |
| L11 | Neo⁺WT⁺ | FNDC3B | FNDC3B_L3M | 0 | 0 | 0 | 29/DV5*01 | CAASGGNSG | | | 13*01 | CASSPLRGPY | 2544 2627 |
| | | | | | | | | YALNF | | | | EQYF | |
| L2 | Neo⁺WT⁺ | FNDC3B | FNDC3B_L3M | 0 | 0 | 0 | | | | | | | |
| M2 | Neo⁺WT⁺ | FNDC3B | FNDC3B_L3M | 0 | 0 | 0 | | | | | 20-1*01 | CSATPRYRG | 2628 |
| | | | | | | | | | | | | YEQYF | |
| M4 | Neo⁺WT⁺ | FNDC3B | FNDC3B_L3M | 0 | 0 | 0 | 12-1*01 | CVVRGSQGN | | | | | 2545 |
| | | | | | | | | LIF | | | | | |
| N8 | Neo⁺WT⁺ | FNDC3B | FNDC3B_L3M | 0 | 0 | 0 | | | | | | | |
| K1 | Neo⁺WT⁺ | FNDC3B_L3M | TEAD1_L8F | FNDC3B | TEAD1_(VLE) | 0 | | | | | 3-1*01 | CASAGPGRN | 2629 |
| | | | | | | | | | | | | QPQHF | |
| M11 | Neo⁺WT⁺ | GANAB | GANAB_S5F | 0 | 0 | 0 | 29/DV5*01 | CAASALSGA | | | 4-2*01 | CASSQGSGAN | 2546 2630 |
| | | | | | | | | NSKLTF | | | | VLTF | |
| G17 | Neo⁺WT⁺ | MLL2 | MLL2_L8H | 0 | 0 | 0 | | | | | | | |
| G9 | Neo⁺WT⁺ | MLL2 | 0 | 0 | 0 | 0 | | | | | 7-9*01 | CASYPISRA | 2631 |
| | | | | | | | | | | | | SYEQYF | |
| H12 | Neo⁺WT⁺ | MLL2 | MLL2_L8H | 0 | 0 | 0 | | | | | | | |
| N2 | Neo⁺WT⁺ | MLL2 | MLL2_L8H | 0 | 0 | 0 | | | | | | | |
| G20 | Neo⁺WT⁺ | MRM1 | NSDHL | NSDHL_A9V | USP28 | 0 | | | | | | | |
| J20 | Neo⁺WT⁺ | MRM1 | NSDHL | NSDHL_A9V | 0 | 0 | | | | | 4-1*01 | CASSQDQNT | 2632 |
| | | | | | | | | | | | | EAFF | |
| H1 | Neo⁺WT⁺ | NSDHL | NSDHL_A9V | MRM1 | 0 | 0 | | | | | | | |
| H16 | Neo⁺WT⁺ | NSDHL | NSDHL_A9V | 0 | 0 | 0 | | | | | | | |
| H20 | Neo⁺WT⁺ | NSDHL | NSDHL_A9V | 0 | 0 | 0 | | | | | | | |
| H3 | Neo⁺WT⁺ | NSDHL | NSDHL_A9V | 0 | 0 | 0 | | | | | 7-9*01 | CASSGQGHP | 2633 |
| | | | | | | | | | | | | YNEQFF | |
| H7 | Neo⁺WT⁺ | NSDHL | NSDHL_A9V | 0 | 0 | 0 | | | | | | | |
| I16 | Neo⁺WT⁺ | NSDHL | NSDHL_A9V | 0 | 0 | 0 | | | | | | | |
| I17 | Neo⁺WT⁺ | NSDHL | NSDHL_A9V | 0 | 0 | 0 | | | | | | | |
| I19 | Neo⁺WT⁺ | NSDHL | NSDHL_A9V | 0 | 0 | 0 | | | | | | | |
| I2 | Neo⁺WT⁺ | NSDHL | NSDHL_A9V | 0 | 0 | 0 | | | | | | | |
| I22 | Neo⁺WT⁺ | NSDHL | NSDHL_A9V | 0 | 0 | 0 | | | | | | | |
| I24 | Neo⁺WT⁺ | NSDHL | NSDHL_A9V | 0 | 0 | 0 | | | | | | | |
| I3 | Neo⁺WT⁺ | NSDHL | NSDHL_A9V | 0 | 0 | 0 | 3*01 | CAVRETNPK | | | | | 2547 |
| | | | | | | | | GKLIF | | | | | |
| I5 | Neo⁺WT⁺ | NSDHL | NSDHL_A9V | 0 | 0 | 0 | | | | | | | |
| J10 | Neo⁺WT⁺ | NSDHL | NSDHL_A9V | 0 | 0 | 0 | | | | | | | |
| J21 | Neo⁺WT⁺ | NSDHL | NSDHL_A9V | 0 | 0 | 0 | | | | | | | |
| J24 | Neo⁺WT⁺ | NSDHL | NSDHL_A9V | 0 | 0 | 0 | | | | | | | |
| J8 | Neo⁺WT⁺ | NSDHL | NSDHL_A9V | 0 | 0 | 0 | 9-2*01 | CALSEVNRD | | | 7-9*01 | CASSPMGQSY | 2548 2634 |
| | | | | | | | | DKIIF | | | | EQYF | |
| J9 | Neo⁺WT⁺ | NSDHL | NSDHL_A9V | 0 | 0 | 0 | | | | | | | |
| K10 | Neo⁺WT⁺ | NSDHL | NSDHL_A9V | 0 | 0 | 0 | | | | | | | |
| K11 | Neo⁺WT⁺ | NSDHL | NSDHL_A9V | 0 | 0 | 0 | 14/DV4*01 | CAMRELDGQ | | | 9*01 | CASSTGGTSG | 2549 2635 |
| | | | | | | | | KLLF | | | | GRNTGELFF | |
| K13 | Neo⁺WT⁺ | NSDHL | NSDHL_A9V | 0 | 0 | 0 | | | | | | | |
| K17 | Neo⁺WT⁺ | NSDHL | NSDHL_A9V | 0 | 0 | 0 | | | | | | | |
| K4 | Neo⁺WT⁺ | NSDHL | NSDHL_A9V | 0 | 0 | 0 | | | | | | | |
| L15 | Neo⁺WT⁺ | NSDHL | NSDHL_A9V | 0 | 0 | 0 | | | | | | | |
| L5 | Neo⁺WT⁺ | NSDHL | NSDHL_A9V | 0 | 0 | 0 | | | | | | | |
| L8 | Neo⁺WT⁺ | NSDHL | NSDHL_A9V | 0 | 0 | 0 | | | | | 20-1*01 | CSARGDPNY | 2636 |
| | | | | | | | | | | | | EQYF | |
| M1 | Neo⁺WT⁺ | NSDHL | NSDHL_A9V | 0 | 0 | 0 | | | | | | | |
| M10 | Neo⁺WT⁺ | NSDHL | NSDHL_A9V | 0 | 0 | 0 | 19*01 | CALSEANYG | | | 4-1*01 | CASSPRAYNE | 2550 2637 |
| | | | | | | | | GSQGNLIF | | | | QFF | |
| N1 | Neo⁺WT⁺ | NSDHL | NSDHL_A9V | 0 | 0 | 0 | 1-2*01 | CAVRGLTGA | | | | | 2551 |
| | | | | | | | | NNLFF | | | | | |
| G22 | Neo⁺WT⁺ | NSDHL_A9V | NSDHL | 0 | 0 | 0 | 38-1*01 | CAFMMDNNN | | | 9*01 | CASSGQGGDE | 2552 2638 |
| | | | | | | | | DMRF | | | | QYF | |
| H14 | Neo⁺WT⁺ | NSDHL_A9V | NSDHL | 0 | 0 | 0 | 10*01 | CVVTPTDSW | | | | | 2553 |
| | | | | | | | | GKLQF | | | | | |
| H17 | Neo⁺WT⁺ | NSDHL_A9V | NSDHL | 0 | 0 | 0 | 9-2*01 | CALSEGQTG | | | 9*01 | CASSVGGGSS | 2554 2639 |
| | | | | | | | | ANNLFF | | | | YEQYF | |
| H6 | Neo⁺WT⁺ | NSDHL_A9V | NSDHL | 0 | 0 | 0 | | | | | | | |
| I7 | Neo⁺WT⁺ | NSDHL_A9V | NSDHL | 0 | 0 | 0 | | | | | | | |
| I8 | Neo⁺WT⁺ | NSDHL_A9V | NSDHL | 0 | 0 | 0 | 5*01 | CAESRPEYG | | | | | 2555 |
| | | | | | | | | NKLVF | | | | | |
| I9 | Neo⁺WT⁺ | NSDHL_A9V | NSDHL | 0 | 0 | 0 | 14/DV4*01 | CAMRAYSGG | | | 9*01 | CASSVASGGY | 2556 2640 |
| | | | | | | | | GADGLTF | | | | TDTQYF | |
| J13 | Neo⁺WT⁺ | NSDHL_A9V | NSDHL | 0 | 0 | 0 | | | | | | | |
| J19 | Neo⁺WT⁺ | NSDHL_A9V | NSDHL | 0 | 0 | 0 | | | | | | | |
| J23 | Neo⁺WT⁺ | NSDHL_A9V | NSDHL | 0 | 0 | 0 | 24*01 | CAPPGAQKL | | | | | 2557 |
| | | | | | | | | VF | | | | | |
| K15 | Neo⁺WT⁺ | NSDHL_A9V | NSDHL | 0 | 0 | 0 | 12-3*01 | CAMTITGNQ | | | | | 2558 |
| | | | | | | | | FYF | | | | | |
| N3 | Neo⁺WT⁺ | NSDHL_A9V | NSDHL | 0 | 0 | 0 | | | | | | | |
| N7 | Neo⁺WT⁺ | NSDHL_A9V | NSDHL | 0 | 0 | 0 | | | | | 28*01 | CASSRSRWE | 2641 |
| | | | | | | | | | | | | FYGYTF | |
| O2 | Neo⁺WT⁺ | NSDHL_A9V | NSDHL | 0 | 0 | 0 | 19*01 | CALSEAGSG | | | 9*01 | CASNRGYNEQ | 2559 2642 |
| | | | | | | | | NTPLVF | | | | FF | |
| I11 | Neo⁺WT⁺ | PGM5 | PGM5_H5Y | 0 | 0 | 0 | 16*01 | CALIRNSGN | | | | | 2560 |
| | | | | | | | | TPLVF | | | | | |
| J16 | Neo⁺WT⁺ | PGM5 | PGM5_H5Y | 0 | 0 | 0 | | | | | | | |
| K8 | Neo⁺WT⁺ | PGM5 | PGM5_H5Y | 0 | 0 | 0 | 17*01 | CATEDYNTD | | | | | 2561 |
| | | | | | | | | KLIF | | | | | |
| I23 | Neo⁺WT⁺ | PGM5_H5Y | PGM5 | 0 | 0 | 0 | | | | | | | |
| G15 | Neo⁺WT⁺ | SEC24A | SEC24A_P5L | 0 | 0 | 0 | | | | | 4-3*01 | CASSQAERG | 2643 |
| | | | | | | | | | | | | ESYNEQFF | |
| G16 | Neo⁺WT⁺ | SMARCD3 | SMARCD3_H8Y | 0 | 0 | 0 | | | | | | | |
| J4 | Neo⁺WT⁺ | SMARCD3 | SMARCD3_H8Y | 0 | 0 | 0 | | | | | 27*01 | CASSLGGNP | 2644 |
| | | | | | | | | | | | | TYNEQFF | |
| L12 | Neo⁺WT⁺ | SMARCD3 | SMARCD3_H8Y | 0 | 0 | 0 | | | | | | | |
| H24 | Neo⁺WT⁺ | SNX24 | SNX24_P6L | 0 | 0 | 0 | | | | | | | |
| O4 | Neo⁺WT⁺ | TEAD1_(SVL) | TEAD1_L9F | 0 | 0 | 0 | | | | | 2*01 | CASRIPDRN | 2645 |
| | | | | | | | | | | | | EQFF | |
| H5 | Neo⁺WT⁺ | TEAD1_(VLE) | MAGEA12_KMAE | 0 | 0 | 0 | | | | | | | |
| G21 | Neo⁺WT⁺ | WDR46 | WDR46_T3I | 0 | 0 | 0 | 14/DV4*01 | CAMRELNFN | | | | | 2562 |
| | | | | | | | | KFYF | | | | | |
| A5 | Neo⁺WT⁻ | AKAP13_Q8K | 0 | 0 | 0 | 0 | | | | | 30*01 | CAWSAGGTG | 2646 |
| | | | | | | | | | | | | ELFF | |
| B10 | Neo⁺WT⁻ | AKAP13_Q8K | 0 | 0 | 0 | 0 | | | | | | | |
| B14 | Neo⁺WT⁻ | AKAP13_Q8K | 0 | 0 | 0 | 0 | 38-2/DV8*01 | CAYHDNNDM | | | | | 2563 |
| | | | | | | | | RF | | | | | |
| D13 | Neo⁺WT⁻ | AKAP13_Q8K | 0 | 0 | 0 | 0 | | | | | 30*01 | CAWMGSYNE | 2647 |
| | | | | | | | | | | | | QFF | |
| E4 | Neo⁺WT⁻ | AKAP13_Q8K | 0 | 0 | 0 | 0 | 29/DV5*01 | CAASAMDSS | | | | | 2564 |
| | | | | | | | | YKLIF | | | | | |
| F11 | Neo⁺WT⁻ | AKAP13_Q8K | 0 | 0 | 0 | 0 | | | | | | | |
| F19 | Neo⁺WT⁻ | AKAP13_Q8K | 0 | 0 | 0 | 0 | | | | | | | |
| E6 | Neo⁺WT⁻ | ERBB2_H8Y | 0 | 0 | 0 | 0 | | | | | | | |
| E12 | Neo⁺WT⁻ | FNDC3B_L3M | 0 | 0 | 0 | 0 | | | | | 7-6*01 | CASSLQGSY | 2648 |
| | | | | | | | | | | | | EQYF | |
| A18 | Neo⁺WT⁻ | GANAB_S5F | 0 | 0 | 0 | 0 | | | | | | | |
| A21 | Neo⁺WT⁻ | GANAB_S5F | 0 | 0 | 0 | 0 | | | | | | | |
| A6 | Neo⁺WT⁻ | GANAB_S5F | 0 | 0 | 0 | 0 | 19*01 | CALSEAEYN | | | 20-1*01 | CSARPGLAGG | 2565 2649 |
| | | | | | | | | FNKFYF | | | | YEQYF | |
| A9 | Neo⁺WT⁻ | GANAB_S5F | 0 | 0 | 0 | 0 | | | | | 6-5*01 | CASSYQTGN | 2650 |
| | | | | | | | | | | | | EQFF | |
| B24 | Neo⁺WT⁻ | GANAB_S5F | 0 | 0 | 0 | 0 | 35*01 | CAGQSRYNR | | | | | 2566 |
| | | | | | | | | DDKIIF | | | | | |
| B4 | Neo⁺WT⁻ | GANAB_S5F | 0 | 0 | 0 | 0 | 12-2*01 | CAAAAGANN | | | | | 2567 |
| | | | | | | | | LFF | | | | | |
| B5 | Neo⁺WT⁻ | GANAB_S5F | 0 | 0 | 0 | 0 | 25*01 | CAGGSNDYK | | | | | 2568 |
| | | | | | | | | LSF | | | | | |
| B8 | Neo⁺WT⁻ | GANAB_S5F | 0 | 0 | 0 | 0 | 1-1*01 | CAVSFYNQG | | | 19*01 | CASRGSGAST | 2569 2651 |
| | | | | | | | | GKLIF | | | | GELFF | |
| B9 | Neo⁺WT⁻ | GANAB_S5F | 0 | 0 | 0 | 0 | | | | | | | |
| C10 | Neo⁺WT⁻ | GANAB_S5F | 0 | 0 | 0 | 0 | | | | | | | |
| C11 | Neo⁺WT⁻ | GANAB_S5F | 0 | 0 | 0 | 0 | | | | | | | |
| C2 | Neo⁺WT⁻ | GANAB_S5F | 0 | 0 | 0 | 0 | | | | | | | |
| C22 | Neo⁺WT⁻ | GANAB_S5F | 0 | 0 | 0 | 0 | | | | | 9*01 | CASSGQGTD | 2652 |
| | | | | | | | | | | | | TQYF | |
| C3 | Neo⁺WT⁻ | GANAB_S5F | 0 | 0 | 0 | 0 | | | | | | | |
| C5 | Neo⁺WT⁻ | GANAB_S5F | 0 | 0 | 0 | 0 | | | | | 7-9*01 | CASSLWAEP | 2653 |
| | | | | | | | | | | | | DTQYF | |
| C6 | Neo⁺WT⁻ | GANAB_S5F | 0 | 0 | 0 | 0 | | | | | | | |
| D14 | Neo⁺WT⁻ | GANAB_S5F | 0 | 0 | 0 | 0 | | | | | | | |
| D19 | Neo⁺WT⁻ | GANAB_S5F | 0 | 0 | 0 | 0 | | | | | | | |
| D2 | Neo⁺WT⁻ | GANAB_S5F | 0 | 0 | 0 | 0 | | | | | | | |
| D4 | Neo⁺WT⁻ | GANAB_S5F | 0 | 0 | 0 | 0 | | | | | | | |
| E16 | Neo⁺WT⁻ | GANAB_S5F | 0 | 0 | 0 | 0 | | | | | | | |
| E8 | Neo⁺WT⁻ | GANAB_S5F | 0 | 0 | 0 | 0 | 8-1*01 | CAVNAPDTD | | | | | 2570 |
| | | | | | | | | KLIF | | | | | |
| E9 | Neo⁺WT⁻ | GANAB_S5F | 0 | 0 | 0 | 0 | 39*01 | CAVVNSNSG | | | | | 2571 |
| | | | | | | | | YALNF | | | | | |
| F13 | Neo⁺WT⁻ | GANAB_S5F | 0 | 0 | 0 | 0 | | | | | | | |
| F17 | Neo⁺WT⁻ | GANAB_S5F | 0 | 0 | 0 | 0 | | | | | | | |
| B22 | Neo⁺WT⁻ | GCN1L1_L6P | 0 | 0 | 0 | 0 | | | | | | | |
| A1 | Neo⁺WT⁻ | GNL3L_R4C | 0 | 0 | 0 | 0 | | | | | | | |
| A11 | Neo⁺WT⁻ | GNL3L_R4C | 0 | 0 | 0 | 0 | | | | | | | |
| A13 | Neo⁺WT⁻ | GNL3L_R4C | 0 | 0 | 0 | 0 | | | | | | | |
| A15 | Neo⁺WT⁻ | GNL3L_R4C | 0 | 0 | 0 | 0 | | | | | | | |
| A16 | Neo⁺WT⁻ | GNL3L_R4C | 0 | 0 | 0 | 0 | 21*01 | CAVLLNNAG | | | | | 2572 |
| | | | | | | | | NMLTF | | | | | |
| A17 | Neo⁺WT⁻ | GNL3L_R4C | 0 | 0 | 0 | 0 | | | | | 6-5*01 | CASSLGISY | 2654 |
| | | | | | | | | | | | | EQYF | |
| A2 | Neo⁺WT⁻ | GNL3L_R4C | 0 | 0 | 0 | 0 | | | | | 4-3*01 | CASSQVTGY | 2655 |
| | | | | | | | | | | | | EQYF | |
| A20 | Neo⁺WT⁻ | GNL3L_R4C | 0 | 0 | 0 | 0 | | | | | | | |
| A23 | Neo⁺WT⁻ | GNL3L_R4C | 0 | 0 | 0 | 0 | | | | | | | |
| A3 | Neo⁺WT⁻ | GNL3L_R4C | 0 | 0 | 0 | 0 | 20*01 | CAVSGGYRD | | | 4-1*01 | CASSQVSGGS | 2573 2656 |
| | | | | | | | | DKIIF | | | | YEQYF | |
| A4 | Neo⁺WT⁻ | GNL3L_R4C | 0 | 0 | 0 | 0 | 26-1*01 | CIVRDWANF | 13-1*01 | CAASIDRDDK | | | 2574 2613 |
| | | | | | | | | GNEKLTF | | IIF | | | |
| B13 | Neo⁺WT⁻ | GNL3L_R4C | 0 | 0 | 0 | 0 | | | | | | | |
| B16 | Neo⁺WT⁻ | GNL3L_R4C | 0 | 0 | 0 | 0 | | | | | | | |
| B17 | Neo⁺WT⁻ | GNL3L_R4C | 0 | 0 | 0 | 0 | 26-2*01 | CILTMGTSY | | | 4-3*01 | CASSQEPSGF | 2575 2657 |
| | | | | | | | | DKVIF | | | | YEQYF | |
| B18 | Neo⁺WT⁻ | GNL3L_R4C | 0 | 0 | 0 | 0 | 12-2*01 | CAVNEATGR | | | | | 2576 |
| | | | | | | | | RALTF | | | | | |
| B19 | Neo⁺WT⁻ | GNL3L_R4C | 0 | 0 | 0 | 0 | 22*01 | CAVDPNTGN | | | 4-2*01 | CASSQQGSEQ | 2577 2658 |
| | | | | | | | | QFYF | | | | YF | |
| B2 | Neo⁺WT⁻ | GNL3L_R4C | 0 | 0 | 0 | 0 | | | | | | | |
| B20 | Neo⁺WT⁻ | GNL3L_R4C | 0 | 0 | 0 | 0 | | | | | | | |
| B21 | Neo⁺WT⁻ | GNL3L_R4C | 0 | 0 | 0 | 0 | | | | | 7-6*01 | CASSLGEDY | 2659 |
| | | | | | | | | | | | | EQYF | |
| B23 | Neo⁺WT⁻ | GNL3L_R4C | 0 | 0 | 0 | 0 | | | | | | | |
| B6 | Neo⁺WT⁻ | GNL3L_R4C | 0 | 0 | 0 | 0 | | | | | | | |
| C12 | Neo⁺WT⁻ | GNL3L_R4C | 0 | 0 | 0 | 0 | | | | | | | |
| C13 | Neo⁺WT⁻ | GNL3L_R4C | 0 | 0 | 0 | 0 | | | | | | | |
| C14 | Neo⁺WT⁻ | GNL3L_R4C | 0 | 0 | 0 | 0 | | | | | 29-1*01 | CSVQGPYNE | 2660 |
| | | | | | | | | | | | | QFF | |
| C16 | Neo⁺WT⁻ | GNL3L_R4C | 0 | 0 | 0 | 0 | | | | | | | |
| C19 | Neo⁺WT⁻ | GNL3L_R4C | 0 | 0 | 0 | 0 | 39*01 | CAADTSGTY | | | | | 2578 |
| | | | | | | | | KYIF | | | | | |
| C21 | Neo⁺WT⁻ | GNL3L_R4C | 0 | 0 | 0 | 0 | | | | | | | |
| C24 | Neo⁺WT⁻ | GNL3L_R4C | 0 | 0 | 0 | 0 | 13-1*01 | CAATRDYKL | | | | | 2579 |
| | | | | | | | | SF | | | | | |
| C4 | Neo⁺WT⁻ | GNL3L_R4C | 0 | 0 | 0 | 0 | | | | | | | |
| C9 | Neo⁺WT⁻ | GNL3L_R4C | 0 | 0 | 0 | 0 | 19*01 | CALAGWEYG | | | 4-3*01 | CASSPGQGID | 2580 2661 |
| | | | | | | | | NKLVF | | | | SPLHF | |
| D12 | Neo⁺WT⁻ | GNL3L_R4C | 0 | 0 | 0 | 0 | | | | | | | |
| D15 | Neo⁺WT⁻ | GNL3L_R4C | 0 | 0 | 0 | 0 | | | | | | | |
| D16 | Neo⁺WT⁻ | GNL3L_R4C | 0 | 0 | 0 | 0 | | | | | | | |
| D18 | Neo⁺WT⁻ | GNL3L_R4C | 0 | 0 | 0 | 0 | | | | | | | |
| D21 | Neo⁺WT⁻ | GNL3L_R4C | 0 | 0 | 0 | 0 | | | | | | | |
| D23 | Neo⁺WT⁻ | GNL3L_R4C | 0 | 0 | 0 | 0 | 12-1*01 | CVVNINSGN | | | | | 2581 |
| | | | | | | | | TPLVF | | | | | |
| D24 | Neo⁺WT⁻ | GNL3L_R4C | 0 | 0 | 0 | 0 | | | | | | | |
| D3 | Neo⁺WT⁻ | GNL3L_R4C | 0 | 0 | 0 | 0 | | | | | | | |
| D5 | Neo⁺WT⁻ | GNL3L_R4C | 0 | 0 | 0 | 0 | 13-1*01 | CAAEGNTGG | | | | | 2582 |
| | | | | | | | | FKTIF | | | | | |
| D6 | Neo⁺WT⁻ | GNL3L_R4C | 0 | 0 | 0 | 0 | | | | | | | |
| D8 | Neo⁺WT⁻ | GNL3L_R4C | 0 | 0 | 0 | 0 | | | | | 6-5*01 | CASSYSGGY | 2662 |
| | | | | | | | | | | | | EQYF | |
| E10 | Neo⁺WT⁻ | GNL3L_R4C | 0 | 0 | 0 | 0 | | | | | | | |
| E15 | Neo⁺WT⁻ | GNL3L_R4C | 0 | 0 | 0 | 0 | | | | | | | |
| E17 | Neo⁺WT⁻ | GNL3L_R4C | 0 | 0 | 0 | 0 | | | | | | | |
| E18 | Neo⁺WT⁻ | GNL3L_R4C | 0 | 0 | 0 | 0 | | | | | | | |
| E21 | Neo⁺WT⁻ | GNL3L_R4C | 0 | 0 | 0 | 0 | | | | | | | |
| E22 | Neo⁺WT⁻ | GNL3L_R4C | 0 | 0 | 0 | 0 | | | | | 30*01 | CAWIRTGGY | 2663 |
| | | | | | | | | | | | | GYTF | |
| E23 | Neo⁺WT⁻ | GNL3L_R4C | 0 | 0 | 0 | 0 | | | | | | | |
| E7 | Neo⁺WT⁻ | GNL3L_R4C | 0 | 0 | 0 | 0 | | | | | | | |
| F1 | Neo⁺WT⁻ | GNL3L_R4C | 0 | 0 | 0 | 0 | | | | | | | |
| F10 | Neo⁺WT⁻ | GNL3L_R4C | 0 | 0 | 0 | 0 | | | | | | | |
| F12 | Neo⁺WT⁻ | GNL3L_R4C | 0 | 0 | 0 | 0 | | | | | | | |
| F14 | Neo⁺WT⁻ | GNL3L_R4C | 0 | 0 | 0 | 0 | | | | | | | |
| F16 | Neo⁺WT⁻ | GNL3L_R4C | 0 | 0 | 0 | 0 | | | | | | | |
| F18 | Neo⁺WT⁻ | GNL3L_R4C | 0 | 0 | 0 | 0 | | | | | | | |
| F2 | Neo⁺WT⁻ | GNL3L_R4C | 0 | 0 | 0 | 0 | | | | | | | |
| F20 | Neo⁺WT⁻ | GNL3L_R4C | 0 | 0 | 0 | 0 | | | | | | | |
| F21 | Neo⁺WT⁻ | GNL3L_R4C | 0 | 0 | 0 | 0 | 38-2/DV8*01F | CAAETSGSR | | | | | 2583 |
| | | | | | | | | LTF | | | | | |
| F22 | Neo⁺WT⁻ | GNL3L_R4C | 0 | 0 | 0 | 0 | 12-2*01 | CAVIDGAGS | | | | | 2584 |
| | | | | | | | | YQLTF | | | | | |
| F4 | Neo⁺WT⁻ | GNL3L_R4C | 0 | 0 | 0 | 0 | 12-2*01 | CAVFSGGYQ | | | 12-3*01, | CASSPGGGYE | 2585 2664 |
| | | | | | | | | KVTF | | | 12-4*01 | QYF | |
| F6 | Neo⁺WT⁻ | GNL3L_R4C | 0 | 0 | 0 | 0 | | | | | | | |
| A12 | Neo⁺WT⁻ | MAGEA6_KVAK | 0 | 0 | 0 | 0 | | | | | | | |
| A19 | Neo⁺WT⁻ | MAGEA6_KVAK | 0 | 0 | 0 | 0 | | | | | | | |
| A10 | Neo⁺WT⁻ | MLL2_L8H | 0 | 0 | 0 | 0 | 9-2*01 | CALRLSSGG | | | 2*01 | CASSFTVAGE | 2586 2665 |
| | | | | | | | | SNYKLTF | | | | QYF | |
| A24 | Neo⁺WT⁻ | MLL2_L8H | 0 | 0 | 0 | 0 | | | | | 6-6*01 | CASSYSGHN | 2666 |
| | | | | | | | | | | | | EQFF | |
| A7 | Neo⁺WT⁻ | MLL2_L8H | 0 | 0 | 0 | 0 | | | | | 4-1*01 | CASSYTIGN | 2667 |
| | | | | | | | | | | | | EQYF | |
| B1 | Neo⁺WT⁻ | MLL2_L8H | 0 | 0 | 0 | 0 | | | | | 10-3*01 | CAISDPDRG | 2668 |
| | | | | | | | | | | | | GRAFF | |
| C8 | Neo⁺WT⁻ | MLL2_L8H | 0 | 0 | 0 | 0 | | | | | | | |
| D11 | Neo⁺WT⁻ | MLL2_L8H | 0 | 0 | 0 | 0 | | | | | | | |
| D22 | Neo⁺WT⁻ | MLL2_L8H | 0 | 0 | 0 | 0 | 13-1*01 | CAAERGNNA | | | | | 2587 |
| | | | | | | | | RLMF | | | | | |
| E13 | Neo⁺WT⁻ | MLL2_L8H | 0 | 0 | 0 | 0 | | | | | | | |
| E19 | Neo⁺WT⁻ | MLL2_L8H | 0 | 0 | 0 | 0 | | | | | | | |
| F5 | Neo⁺WT⁻ | MLL2_L8H | 0 | 0 | 0 | 0 | | | | | | | |
| F3 | Neo⁺WT⁻ | NSDHL_A9V | 0 | 0 | 0 | 0 | 12-2*01 | CAVNPLEGG | | | | | 2588 |
| | | | | | | | | YNKLIF | | | | | |
| D20 | Neo⁺WT⁻ | PGM5_H5Y | 0 | 0 | 0 | 0 | | | | | | | |
| D9 | Neo⁺WT⁻ | PGM5_H5Y | 0 | 0 | 0 | 0 | | | | | | | |
| A14 | Neo⁺WT⁻ | SEC24A_P5L | 0 | 0 | 0 | 0 | | | | | 6-5*01 | CASTAGGGT | 2669 |
| | | | | | | | | | | | | DTQYF | |
| A22 | Neo⁺WT⁻ | SEC24A_P5L | 0 | 0 | 0 | 0 | | | | | 6-5*01 | CASSYSPGA | 2670 |
| | | | | | | | | | | | | YTEAFF | |
| A8 | Neo⁺WT⁻ | SEC24A_P5L | 0 | 0 | 0 | 0 | | | | | 29-1*01 | CSVWKENAF | 2671 |
| | | | | | | | | | | | | EQFF | |
| B11 | Neo⁺WT⁻ | SEC24A_P5L | 0 | 0 | 0 | 0 | | | | | | | |
| B15 | Neo⁺WT⁻ | SEC24A_P5L | 0 | 0 | 0 | 0 | | | | | | | |
| C1 | Neo⁺WT⁻ | SEC24A_P5L | 0 | 0 | 0 | 0 | | | | | | | |
| C15 | Neo⁺WT⁻ | SEC24A_P5L | 0 | 0 | 0 | 0 | | | | | | | |
| C17 | Neo⁺WT⁻ | SEC24A_P5L | 0 | 0 | 0 | 0 | | | | | | | |
| C23 | Neo⁺WT⁻ | SEC24A_P5L | 0 | 0 | 0 | 0 | 17*01 | CATDRNAPY | | | 4-3*01 | CASSQDTGYE | 2589 2672 |
| | | | | | | | | ALNF | | | | QYF | |
| C7 | Neo⁺WT⁻ | SEC24A_P5L | 0 | 0 | 0 | 0 | 17*01 | CATDEGNTP | | | 12-3*01, | CASGLDTQYF | 2590 2673 |
| | | | | | | | | LVF | | | 12-4*01 | | |
| D1 | Neo⁺WT⁻ | SEC24A_P5L | 0 | 0 | 0 | 0 | | | | | | | |
| D10 | Neo⁺WT⁻ | SEC24A_P5L | 0 | 0 | 0 | 0 | | | | | | | |
| E14 | Neo⁺WT⁻ | SEC24A_P5L | 0 | 0 | 0 | 0 | | | | | | | |
| E3 | Neo⁺WT⁻ | SEC24A_P5L | 0 | 0 | 0 | 0 | | | | | | | |
| F15 | Neo⁺WT⁻ | SEC24A_P5L | 0 | 0 | 0 | 0 | | | | | | | |
| F23 | Neo⁺WT⁻ | SEC24A_P5L | 0 | 0 | 0 | 0 | 39*01 | CAVDGGEYG | | | | | 2591 |
| | | | | | | | | NKLVF | | | | | |
| F24 | Neo⁺WT⁻ | SEC24A_P5L | 0 | 0 | 0 | 0 | | | | | 28*01 | CASSLTGVD | 2674 |
| | | | | | | | | | | | | GYTF | |
| F8 | Neo⁺WT⁻ | SEC24A_P5L | 0 | 0 | 0 | 0 | 17*01 | CATDDTGGF | | | | | 2592 |
| | | | | | | | | KTIF | | | | | |
| F9 | Neo⁺WT⁻ | SEC24A_P5L | 0 | 0 | 0 | 0 | 38-2/DV8*01 | CAYNPDMRF | | | 20-1*01 | CSAAYNTFGE | 2593 2675 |
| | | | | | | | | | | | | QFF | |
| C20 | Neo⁺WT⁻ | SMARCD3_H8Y | 0 | 0 | 0 | 0 | | | | | | | |
| E2 | Neo⁺WT⁻ | SMARCD3_H8Y | 0 | 0 | 0 | 0 | | | | | | | |
| E24 | Neo⁺WT⁻ | SMARCD3_H8Y | 0 | 0 | 0 | 0 | 8-2*01 | CVVSLHTGG | | | | | 2594 |
| | | | | | | | | FKTIF | | | | | |
| F7 | Neo⁺WT⁻ | SMARCD3_H8Y | 0 | 0 | 0 | 0 | | | | | | | |
| D17 | Neo⁺WT⁻ | SNX24_P6L | 0 | 0 | 0 | 0 | | | | | 20-1*01 | CSATSGTDT | 2676 |
| | | | | | | | | | | | | QYF | |
| D7 | Neo⁺WT⁻ | SNX24_P6L | 0 | 0 | 0 | 0 | 13-2*01 | CAENVTGNQ | | | 13*01 | CASSLGGFAG | 2595 2677 |
| | | | | | | | | FYF | | | | NTIYF | |
| E1 | Neo⁺WT⁻ | SNX24_P6L | 0 | 0 | 0 | 0 | 38-2/DV8*01 | CASKRGGAD | | | | | 2596 |
| | | | | | | | | GLTF | | | | | |
| C18 | Neo⁺WT⁻ | USP28_C5F | 0 | 0 | 0 | 0 | | | | | | | |
| E20 | Neo⁺WT⁻ | USP28_C5F | 0 | 0 | 0 | 0 | | | | | | | |
| B12 | Neo⁺WT⁻ | WDR46_T3I | 0 | 0 | 0 | 0 | | | | | | | |
| B3 | Neo⁺WT⁻ | WDR46_T3I | 0 | 0 | 0 | 0 | | | | | | | |
| B7 | Neo⁺WT⁻ | WDR46_T3I | 0 | 0 | 0 | 0 | | | | | | | |
| E11 | Neo⁺WT⁻ | WDR46_T3I | 0 | 0 | 0 | 0 | | | | | 29-1*01 | CSSPGREGP | 2678 |
| | | | | | | | | | | | | QYF | |
| E5 | Neo⁺WT⁻ | WDR46_T3I | 0 | 0 | 0 | 0 | | | | | | | |
| G5 | Neo⁻WT⁺ | AKAP13 | 0 | 0 | 0 | 0 | | | | | | | |
| H21 | Neo⁻WT⁺ | AKAP13 | 0 | 0 | 0 | 0 | 38-2/DV8*01 | CAYSPPLVF | | | 6-2*01, | CASRGGDGET | 2597 2679 |
| | | | | | | | | | | | 6-3*01 | QYF | |
| J11 | Neo⁻WT⁺ | AKAP13 | 0 | 0 | 0 | 0 | 38-2/DV8*01 | CAFAPGNNN | | | | | 2598 |
| | | | | | | | | DMRF | | | | | |
| K9 | Neo⁻WT⁺ | AKAP13 | 0 | 0 | 0 | 0 | 38-1*01 | CAYFPYGQN | | | 9*01 | CASGDSGALE | 2599 2680 |
| | | | | | | | | FVF | | | | FF | |
| L4 | Neo⁻WT⁺ | AKAP13 | 0 | 0 | 0 | 0 | | | | | | | |
| H19 | Neo⁻WT⁺ | COL18A1 | 0 | 0 | 0 | 0 | | | | | 19*01 | CASSSAGTE | 2681 |
| | | | | | | | | | | | | AFF | |
| L14 | Neo⁻WT⁺ | COL18A1 | 0 | 0 | 0 | 0 | 14/DV4*01 | CAMRVSDNF | | | | | 2600 |
| | | | | | | | | NKFYF | | | | | |
| G11 | Neo⁻WT⁺ | FNDC3B | 0 | 0 | 0 | 0 | | | | | | | |
| G1 | Neo⁻WT⁺ | GANAB | 0 | 0 | 0 | 0 | | | | | | | |
| G10 | Neo⁻WT⁺ | GANAB | 0 | 0 | 0 | 0 | | | | | | | |
| H15 | Neo⁻WT⁺ | GANAB | 0 | 0 | 0 | 0 | | | | | | | |
| H22 | Neo⁻WT⁺ | GANAB | 0 | 0 | 0 | 0 | | | | | | | |
| I21 | Neo⁻WT⁺ | GANAB | 0 | 0 | 0 | 0 | | | | | 4-3*01 | CASSQGGGG | 2682 |
| | | | | | | | | | | | | TDTQYF | |
| J14 | Neo⁻WT⁺ | GANAB | 0 | 0 | 0 | 0 | | | | | | | |
| J2 | Neo⁻WT⁺ | GANAB | 0 | 0 | 0 | 0 | 5*01 | CAESPSNFG | | | | | 2601 |
| | | | | | | | | NEKLTF | | | | | |
| L16 | Neo⁻WT⁺ | GANAB | 0 | 0 | 0 | 0 | | | | | | | |
| L3 | Neo⁻WT⁺ | GANAB | 0 | 0 | 0 | 0 | | | | | | | |
| M5 | Neo⁻WT⁺ | GANAB | 0 | 0 | 0 | 0 | | | | | | | |
| N4 | Neo⁻WT⁺ | GANAB | 0 | 0 | 0 | 0 | 13-2*01 | CAENPCSND | | | | | 2602 |
| | | | | | | | | YKLSF | | | | | |
| K14 | Neo⁻WT⁺ | MAGEA3_KVAE | 0 | 0 | 0 | 0 | | | | | 19*01 | CATWDSGNI | 2683 |
| | | | | | | | | | | | | QYF | |
| J15 | Neo⁻WT⁺ | MLL2 | 0 | 0 | 0 | 0 | 12-2*01 | CAVTSNTGK | | | | | 2603 |
| | | | | | | | | LIF | | | | | |
| J6 | Neo⁻WT⁺ | MLL2 | 0 | 0 | 0 | 0 | | | | | | | |
| K16 | Neo⁻WT⁺ | MLL2 | 0 | 0 | 0 | 0 | | | | | 20-1*01 | CSATCNGTF | 2684 |
| | | | | | | | | | | | | LYQETQYF | |
| M9 | Neo⁻WT⁺ | MLL2 | 0 | 0 | 0 | 0 | 14/DV4*01 | CAMREDYSS | | | 4-3*01 | CASSQGPPGS | 2604 2685 |
| | | | | | | | | ASKIIF | | | | GGGNEQFF | |
| I4 | Neo⁻WT⁺ | MRM1 | 0 | 0 | 0 | 0 | 12-3*01 | CAMALGNTG | | | | | 2605 |
| | | | | | | | | NQFYF | | | | | |
| J7 | Neo⁻WT⁺ | MRM1 | 0 | 0 | 0 | 0 | | | | | | | |
| K7 | Neo⁻WT⁺ | MRM1 | 0 | 0 | 0 | 0 | 12-2*01 | CAASGGGAD | | | | | 2606 |
| | | | | | | | | GLTF | | | | | |
| L7 | Neo⁻WT⁺ | MRM1 | GANAB | 0 | 0 | 0 | | | | | | | |
| L9 | Neo⁻WT⁺ | MRM1 | 0 | 0 | 0 | 0 | | | | | | | |
| N5 | Neo⁻WT⁺ | MRM1 | 0 | 0 | 0 | 0 | 12-2*01 | CAGYSGGGA | | | 6-5*01 | CASSSLGDSY | 2607 2686 |
| | | | | | | | | DGLTF | | | | EQYF | |
| N9 | Neo⁻WT⁺ | MRM1 | 0 | 0 | 0 | 0 | 12-2*01 | CAVNGNQFY | | | 12-3*01, | CASSLGGPGA | 2608 2687 |
| | | | | | | | | F | | | 12-4*01 | FF | |
| G8 | Neo⁻WT⁺ | PGM5 | 0 | 0 | 0 | 0 | 35*01 | CEGNNNDMR | 19*01 | CALTTDSNSG | | | 2609 2614 |
| | | | | | | | | F | | YALNF | | | |
| N6 | Neo⁻WT⁺ | PGM5 | 0 | 0 | 0 | 0 | | | | | | | |
| G13 | Neo⁻WT⁺ | SEC24A | 0 | 0 | 0 | 0 | | | | | | | |
| I10 | Neo⁻WT⁺ | SEC24A | 0 | 0 | 0 | 0 | | | | | | | |
| K6 | Neo⁻WT⁺ | SEC24A | 0 | 0 | 0 | 0 | | | | | | | |
| M6 | Neo⁻WT⁺ | SEC24A | 0 | 0 | 0 | 0 | 22*01 | CAVAHARLM | | | 6-2*01, | CASSSDINYG | 2610 2688 |
| | | | | | | | | F | | | 6-3*01 | YTF | |
| M8 | Neo⁻WT⁺ | SEC24A | 0 | 0 | 0 | 0 | | | | | 6-5*01 | CASSYSSGY | 2689 |
| | | | | | | | | | | | | GYTF | |
| O3 | Neo⁻WT⁺ | SMARCD3 | 0 | 0 | 0 | 0 | 8-3*01 | CAVGVEYGN | | | | | 2611 |
| | | | | | | | | KLVF | | | | | |
| K18 | Neo⁻WT⁺ | SNX24 | 0 | 0 | 0 | 0 | | | | | | | |
| H18 | Neo⁻WT⁺ | TEAD1_(VLE) | 0 | 0 | 0 | 0 | 24*01 | CAFSQYGNK | | | | | 2612 |
| | | | | | | | | LVF | | | | | |
| J3 | Neo⁻WT⁺ | USP28 | 0 | 0 | 0 | 0 | | | | | | | |
| H13 | Neo⁻WT⁺ | WDR46 | 0 | 0 | 0 | 0 | | | | | | | |
| J22 | Neo⁻WT⁺ | WDR46 | 0 | 0 | 0 | 0 | | | | | | | |

TABLE 8
Description of neoantigen and wildtype peptides used for experiment 5 and 6.

| | | | | | Position | | | Wildtype | | | Mutant |
|---|---|---|---|---|---|---|---|---|---|---|---|
| | | | Wild- | | of | | | HLA-A2 | | | HLA-A2 |
| | | | type | Mutant | mutation | | SEQ | Binding | | SEQ | Binding |
| Wildtype | | HUGO | amino | amino | in | Wildtype | ID | NetMHC | Mutant | ID | NetMHC |
| Name | Mutant Name | symbol | acid | acid | peptide | peptide | NO: | 4.0 (nM) | peptide | NO: | 4.0 (nM) |
| CHST13-VLV | CHST13-VLV_V1M | CHST13 | V | M | 1 | VLVDDAHGL | 2690 | 43.6 | MLVDDAHGL | 2848 | 13 |
| A2ML1-YLD | A2ML1-YLD_K7R | A2ML1 | K | R | 7 | YLDELIKNT | 2691 | 86.4 | YLDELIRNT | 2849 | 71.9 |
| (WT) | | | | | | | | | | | |
| AGFG2-FLQ | AGFG2-FLQ_S4S | AGFG2 | S | F | 4 | FLQSRGNEV | 2692 | 29.6 | FLQFRGNEV | 2850 | 47.7 |
| AGXT2L2-ILT | AGXT2L2-ILT_M5I | AGXT2L2 | M | I | 5 | ILTDMEEKV | 2693 | 75 | ILTDIEEKV | 2851 | 49.5 |
| AHNAK-SMP | AHNAK-SMP_S1F | AHNAK | S | F | 1 | SMPDFDLHL | 2694 | 22.9 | FMPDFDLHL | 2852 | 5.5 |
| AKAP13-KLM | AKAP13-KLM_Q8K | AKAP13 | Q | K | 8 | KLMNIQQQL | 2695 | 15.4 | KLMNIQQKL | 2853 | 20.3 |
| APBB2-GML | APBB2-GML_L3F | APBB2 | L | F | 3 | GMLPVDKPV | 2696 | 31 | GMFPVDKPV | 2854 | 20 |
| APBB2-VQY | APBB2-VQY_L7F | APBB2 | L | F | 7 | VQYLGMLPV | 2697 | 48.3 | VQYLGMFPV | 2855 | 12 |
| APCDD1L-RLP | APCDD1L-RLP_R1W | APCDD1L | R | W | 1 | RLPHVEYEL | 2698 | 51.1 | WLPHVEYEL | 2856 | 24 |
| ATP6AP1-KLG | ATP6AP1-KLG_G3W | ATP6AP1 | G | W | 3 | KLGASPLHV | 2699 | 50.2 | KLWASPLHV | 2857 | 5.5 |
| BAIAP3-ILN | BAIAP3-ILN_V6I | BAIAP3 | V | I | 6 | ILNVDVFTL | 2700 | 38.2 | ILNVDIFTL | 2858 | 26.8 |
| BCL9L-FVY | BCL9L-FVY_T6I | BCL9L | T | I | 6 | FVYVFTTHL | 2701 | 41.8 | FVYVFITHL | 2859 | 45.1 |
| BTBD1-FML | BTBD1-FML_LI | BTBD1 | L | I | 10 | FMLLTQARL | 2702 | 27.6 | FMLLTQARI | 2860 | 33.7 |
| C15orf32- | C15orf32-MLS_G9R | C15orf32 | G | R | 9 | MLSILALVGV | 2703 | 42.6 | MLSILALVRV | 2861 | 90.8 |
| MLS | | | | | | | | | | | |
| C17orf75- | C17orf75-ALS_V7A | C17orf75 | V | A | 7 | ALSYTPVEV | 2704 | 22.7 | ALSYTPAEV | 2862 | 31.8 |
| ALS | | | | | | | | | | | |
| C1S-10 | C1S-10_N1H | C1S | N | H | 1 | NLMDGDLGLI | 2705 | 55.9 | HLMDGDLGLI | 2863 | 50.4 |
| C1S-9 | C1S-9_N1H | C1S | N | H | 1 | NLMDGDLGL | 2706 | 12.9 | HLMDGDLGL | 2864 | 11.8 |
| C3orf58-LMV | C3orf58-LMV_L4P | C3orf58 | L | P | 4 | LMVLHSPSL | 2707 | 50 | LMVPHSPSL | 2865 | 31.9 |
| CAMK1D-KLF | CAMK1D-KLF_K8N | CAMK1D | K | N | 8 | KLFEQILKA | 2708 | 8.6 | KLFEQILNA | 2866 | 6.8 |
| CCM2-YML | CCM2-YML_R6H | CCM2 | R | H | 6 | YMLTLRTKL | 2709 | 36.3 | YMLTLHTKL | 2867 | 14.1 |
| CD47-GLT | CD47-GLT_V6F | CD47 | V | F | 6 | GLTSFVIAI | 2710 | 29.2 | GLTSFFIAI | 2868 | 38.3 |
| CDC37L1-FLS | CDC37L1-FLS_P6L | CDC37L1 | P | L | 6 | FLSDHPYLV | 2711 | 2.5 | FLSDHLYLV | 2869 | 2 |
| CELSR1-YLF | CELSR1-YLF_F3L | CELSR1 | F | L | 3 | YLFAIFSGL | 2712 | 4.5 | YLLAIFSGL | 2870 | 4.9 |
| CHD8-KLN | CHD8-KLN_P7A | CHD8 | P | A | 7 | KLNTITPVV | 2713 | 9 | KLNTITAVV | 2871 | 18.4 |
| CHST14-MLM | CHST14-MLM_F4L | CHST14 | F | L | 4 | MLMFAVIVA | 2714 | 18.5 | MLMLAVIVA | 2872 | 35.9 |
| CLCN4-LLA | CLCN4-LLA_G8V | CLCN4 | G | V | 8 | LLAGTLAGV | 2715 | 9.6 | LLAGTLAVV | 2873 | 17.7 |
| CNKSR1-SLA | CNKSR1-SLA_A9V | CNKSR1 | A | V | 9 | SLAPLSPRA | 2716 | 64.7 | SLAPLSPRV | 2874 | 9.9 |
| COL18A1-VLL | COL18A1-VLL_S8F | COL18A1 | S | F | 8 | VLLGVKLSGV | 2717 | 32.5 | VLLGVKLFGV | 2875 | 9.1 |
| DCHS1-TLF | DCHS1-TLF_I5M | DCHS1 | I | M | 5 | TLFTIVGTV | 2718 | 40.6 | TLFTMVGTV | 2876 | 39.6 |
| DHX33-LLA | DHX33-LLA_M4I | DHX33 | M | I | 4 | LLAMKVPNV | 2719 | 8.3 | LLAIKVPNV | 2877 | 13.7 |
| DHX33-LLA | DHX33-LLA_K5T | DHX33 | K | T | 5 | LLAMKVPNV | 2720 | 8.3 | LLAMTVPNV | 2878 | 8.5 |
| DNAH8-FMT | DNAH8-FMT_G7D | DNAH8 | G | D | 7 | FMTKINGLEV | 2721 | 24.6 | FMTKINDLEV | 2879 | 23.4 |
| DOCK7-FLN | DOCK7-FLN_M9L | DOCK7 | M | L | 9 | FLNDLLSVM | 2722 | 15.1 | FLNDLLSVL | 2880 | 6.3 |
| DOLPP1-GLM | DOLPP1-GLM_A4V | DOLPP1 | A | V | 4 | GLMAIAWFI | 2723 | 3.1 | GLMVIAWFI | 2881 | 7 |
| DRAM1-FII | DRAM1-FII_I3F | DRAM1 | I | F | 3 | FIISYVVAV | 2724 | 3.4 | FIFSYVVAV | 2882 | 3.1 |
| ERBB2-ALI | ERBB2-ALI_H8Y | ERBB2 | H | Y | 8 | ALIHHNTHL | 2725 | 79.3 | ALIHHNTYL | 2883 | 17.9 |
| EXOC3L4-ILL | EXOC3L4-ILL_V91 | EXOC3L4 | V | I | 9 | ILLDWAANV | 2726 | 3.5 | ILLDWAANI | 2884 | 6.3 |
| FAM47B-ALF | FAM47B-ALF_A1S | FAM47B | A | S | 1 | ALFSELSPV | 2727 | 3.9 | SLFSELSPV | 2885 | 3.8 |
| FBXL4-SLL | FBXL4-SLL_L2V | FBXL4 | L | V | 2 | SLLEYYTEL | 2728 | 4.1 | SVLEYYTEL | 2886 | 30.9 |
| FLNA-HIA | FLNA-HIA_P6L | FLNA | P | L | 6 | HIAKSPFEV | 2729 | 93.8 | HIAKSLFEV | 2887 | 21.7 |
| FNDC3B-VVL | FNDC3B-VVL_L3M | FNDC3B | L | M | 3 | VVLSWAPPV | 2730 | 9.6 | VVMSWAPPV | 2888 | 5.8 |
| GABRG3-TAM | GABRG3-TAM_L5I | GABRG3 | L | I | 5 | TAMDLFVTV | 2731 | 33.2 | TAMDIFVTV | 2889 | 27.2 |
| GABRG3-YVT | GABRG3-YVT_L7I | GABRG3 | L | I | 7 | YVTAMDLFV | 2732 | 17.2 | YVTAMDIFV | 2890 | 14.3 |
| GALC-YVV | GALC-YVV_V3L | GALC | V | L | 3 | YVVTWIVGA | 2733 | 47.2 | YVLTWIVGA | 2891 | 14 |
| GANAB-ALY | GANAB-ALY_S5F | GANAB | S | F | 5 | ALYGSVPVL | 2734 | 15.3 | ALYGFVPVL | 2892 | 8.3 |
| GCN1L1-10 | GCN1L1-10_L6P | GCN1L1 | L | P | 6 | ALLETLSLLL | 2735 | 35.7 | ALLETPSLLL | 2893 | 53.5 |
| GCN1L1-9 | GCN1L1-9_L6P | GCN1L1 | L | P | 6 | ALLETLSLL | 2736 | 11 | ALLETPSLL | 2894 | 19.9 |
| GLRA1-LIF | GLRA1-LIF_F6L | GLRA1 | F | L | 6 | LIFNMFYWI | 2737 | 16.2 | LIFNMLYWI | 2895 | 10.9 |
| GOLGA3-SLD | GOLGA3-SLD_P4L | GOLGA3 | P | L | 4 | SLDPTTSPV | 2738 | 10.4 | SLDLTTSPV | 2896 | 19 |
| GPR137B-KMS | GPR137B-KMS_S3P | GPR137B | S | P | 3 | KMSLANIYL | 2739 | 19.1 | KMPLANIYL | 2897 | 38.1 |
| GPR174-FSF | GPR174-FSF_P4S | GPR174 | P | S | 4 | FSFPLDFLV | 2740 | 14.8 | FSFSLDFLV | 2898 | 15 |
| GSTA4-FLQ | GSTA4-FLQ_E4K | GSTA4 | E | K | 4 | FLQEYTVKL | 2741 | 4.2 | FLQKYTVKL | 2899 | 11.7 |
| HAUS3-ILN | HAUS3-ILN_T7A | HAUS3 | T | A | 7 | ILNAMITKI | 2742 | 53 | ILNAMIAKI | 2900 | 48.1 |
| HBZ-KLS | HBZ-KLS_A7T | HBZ | A | T | 7 | KLSELHAYI | 2743 | 11.4 | KLSELHTYI | 2901 | 11.7 |
| HERC1-SLL | HERC1-SLL_PS | HERC1 | P | S | 6 | SLLLLPVSV | 2744 | 16.2 | SLLLLSVSV | 2902 | 17.3 |
| HLA-DRB5- | HLA-DRB5-YMA_KE | HLA-DRB5 | K | E | 4 | YMAKLTVTL | 2745 | 5.6 | YMAELTVTL | 2903 | 3 |
| YMA | | | | | | | | | | | |
| HOXC9-YMY | HOXC9-YMY_G4D | HOXC9 | G | D | 4 | YMYGSPGEL | 2746 | 24.4 | YMYDSPGEL | 2904 | 12.6 |
| HTR1F-10 | HTR1F-10_V1M | HTR1F | V | M | 1 | VMPFSIVYIV | 2747 | 27.5 | MMPFSIVYIV | 2905 | 10.5 |
| HTR1F-9 | HTR1F-9_V1M | HTR1F | V | M | 1 | VMPFSIVYI | 2748 | 31.4 | MMPFSIVYI | 2906 | 10.3 |
| HTR1F-LVM | HTR1F-LVM_V2M | HTR1F | V | M | 2 | LVMPFSIVYI | 2749 | 35.3 | LMMPFSIVYI | 2907 | 5.1 |
| IGF1-TMS | IGF1-TMS_S4F | IGF1 | S | F | 4 | TMSSSHLFYL | 2750 | 14.5 | TMSFSHLFYL | 2908 | 6.1 |
| IL17RA-FIT | IL17RA-FIT_TM | IL17RA | T | M | 3 | FITGISILL | 2751 | 34.8 | FIMGISILL | 2909 | 5.1 |
| INTS1-VLL | INTS1-VLL_L3F | INTS1 | L | F | 3 | VLLHRAFLV | 2752 | 11.3 | VLFHRAFLV | 2910 | 8.6 |
| IPO9-FSS | IPO9-FSS_E4D | IP09 | E | D | 4 | FSSEVLNLV | 2753 | 63.4 | FSSDVLNLV | 2911 | 43.5 |
| ITIH6-RLG | ITIH6-RLG_G3V | ITIH6 | G | V | 3 | RLGPYLEFL | 2754 | 23.4 | RLVPYLEFL | 2912 | 12.6 |
| KAT6A-KLS | KAT6A-KLS_MK | KAT6A | M | K | 7 | KLSREIMPV | 2755 | 5.8 | KLSREIKPV | 2913 | 64.8 |
| KCNB2-LLA | KCNB2-LLA_P6T | KCNB2 | P | T | 6 | LLAILPYYV | 2756 | 5.3 | LLAILTYYV | 2914 | 4.6 |
| KCNC3-FLP | KCNC3-FLP_A7V | KCNC3 | A | V | 7 | FLPDLNANA | 2757 | 21.3 | FLPDLNVNA | 2915 | 14.6 |
| KIF20B-YTS | KIF20B-YTS_S6L | KIF2OB | S | L | 6 | YTSEISSPI | 2758 | 35.4 | YTSEILSPI | 2916 | 14.3 |
| LCP1-NLF | LCP1-NLF_PL | LCP1 | P | L | 7 | NLFNRYPAL | 2759 | 37.3 | NLFNRYLAL | 2917 | 61.6 |
| MAR11-10 | MAR11-10_F1L | MAR11 | F | L | 1 | FLIASVTWLL | 2760 | 4.8 | LLIASVTWLL | 2918 | 15.3 |
| MAR11-9 | MAR11-9_F1L | MAR11 | F | L | 1 | FLIASVTWL | 2761 | 4.3 | LLIASVTWL | 2919 | 15.1 |
| ME1-FLD | ME1-FLD_A8G | ME1 | A | G | 8 | FLDEFMEAV | 2762 | 2.7 | FLDEFMEGV | 2920 | 2.7 |
| MLL2-ALS | MLL2-ALS_L8H | MLL2 | L | H | 8 | ALSPVIPLI | 2763 | 8.1 | ALSPVIPHI | 2921 | 11.3 |
| MPV17-YLW | MPV17-YLW_A5P | MPV17 | A | P | 5 | YLWPAVQLA | 2764 | 5.7 | YLWPPVQLA | 2922 | 9.3 |
| MRGPRF-RLW | MRGPRF-RLW_R1W | MRGPRF | R | W | 1 | RLWEPLRVV | 2765 | 35 | WLWEPLRVV | 2923 | 21.5 |
| MRM1-10 | MRM1-10_T6P | MRM1 | T | P | 6 | LLFGMTPCLL | 2766 | 22.6 | LLFGMPPCLL | 2924 | 34.7 |
| MRM1-9 | MRM1-9_T6P | MRM1 | T | P | 6 | LLFGMTPCL | 2767 | 7.4 | LLFGMPPCL | 2925 | 11.7 |
| MYH4-GLD | MYH4-GLD_D3N | MYH4 | D | N | 3 | GLDETIAKL | 2768 | 30.4 | GLNETIAKL | 2926 | 59.7 |
| MYPN-RVI | MYPN-RVI_R1L | MYPN | R | L | 1 | RVIGMPPPV | 2769 | 36 | LVIGMPPPV | 2927 | 20.7 |
| NBPF24-LLD | NBPF24-LLD_E6G | NBPF24 | E | G | 6 | LLDEKEPEV | 2770 | 13.1 | LLDEKGPEV | 2928 | 12.2 |
| NOS1-FID | NOS1-FID_D3Y | NOS1 | D | Y | 3 | FIDQYYSSI | 2771 | 40.9 | FIYQYYSSI | 2929 | 22.7 |
| NSDHL-ILT | NSDHL-ILT_A9V | NSDHL | A | V | 9 | ILTGLNYEA | 2772 | 41.7 | ILTGLNYEV | 2930 | 7.4 |
| OASL-ILD | OASL-ILD_DN | OASL | D | N | 3 | ILDPADPTL | 2773 | 37 | ILNPADPTL | 2931 | 73.5 |
| OR10A3-ILI | OR10A3-ILI_V6F | OR10A3 | V | F | 6 | ILIVMVPFL | 2774 | 10.4 | ILIVMFPFL | 2932 | 12.3 |
| OR14C36-FML | OR14C36-FML_V6L | OR14C36 | V | L | 6 | FMLYLVTLM | 2775 | 9.5 | FMLYLLTLM | 2933 | 7.6 |
| OR1G1-FLF | OR1G1-FLF_T8M | OR1G1 | T | M | 8 | FLFMYLVTV | 2776 | 3.3 | FLFMYLVMV | 2934 | 3.6 |
| OR2T1-FLN | OR2T1-FLN_F5L | OR2T1 | F | L | 5 | FLNVFFPLL | 2777 | 8.4 | FLNVLFPLL | 2935 | 11.5 |
| OR5K2-YIF | OR5K2-YIF_GE | OR5K2 | G | E | 5 | YIFLGNLAL | 2778 | 23.5 | YIFLENLAL | 2936 | 40.8 |
| OR5M3-KMV | OR5M3-KMV_T8N | OR5M3 | T | N | 8 | KMVAVFYTT | 2779 | 46.2 | KMVAVFYNT | 2937 | 55 |
| OR6F1-VLN | OR6F1-VLN_T8M | OR6F1 | T | M | 8 | VLNPFIYTL | 2780 | 8.8 | VLNPFIYML | 2938 | 10.8 |
| OR8B8-YVN | OR8B8-YVN_V2L | OR8B8 | V | L | 2 | YVNELVVFV | 2781 | 5.9 | YLNELVVFV | 2939 | 2.6 |
| OR8D4-10 | OR8D4-10_G3E | OR8D4 | G | E | 3 | FLGIYTVTVV | 2782 | 26.5 | FLEIYTVTVV | 2940 | 35.9 |
| OR8D4-9 | OR8D4-9_G3E | OR8D4 | G | E | 3 | FLGIYTVTV | 2783 | 8.2 | FLEIYTVTV | 2941 | 17 |
| OR9Q2-FLF | OR9Q2-FLF_S8F | OR9Q2 | S | F | 8 | FLFTFFASI | 2784 | 4.2 | FLFTFFAFI | 2942 | 3.8 |
| OR9Q2-SID | OR9Q2-SID_S1F | OR9Q2 | S | F | 1 | SIDCYLLAI | 2785 | 45.3 | FIDCYLLAI | 2943 | 7.3 |
| OVOL1-SLL | OVOL1-SLL_L9V | OVOL1 | L | V | 9 | SLLQGSPHL | 2786 | 18.2 | SLLQGSPHV | 2944 | 9.5 |
| PABPC1-MLG | PABPC1-MLG_R5Q | PABPC1 | R | Q | 5 | MLGERLFPL | 2787 | 4 | MLGEQLFPL | 2945 | 3.4 |
| PCDHB3-FLF | PCDHB3-FLF_SL | PCDHB3 | S | L | 4 | FLFSVLLFV | 2788 | 2.5 | FLFLVLLFV | 2946 | 5.9 |
| PELP1-LVL | PELP1-LVL_L3F | PELP1 | L | F | 3 | LVLPLVMGV | 2789 | 22.2 | LVFPLVMGV | 2947 | 16.5 |
| PELP1-RLH | PELP1-RLH_L7F | PELP1 | L | F | 7 | RLHDLVLPL | 2790 | 10.6 | RLHDLVFPL | 2948 | 4.7 |
| PGM5-AVG | PGM5-AVG_H5Y | PGM5 | H | Y | 5 | AVGSHVYSV | 2791 | 91.5 | AVGSYVYSV | 2949 | 29.3 |
| PHKA2-LLS | PHKA2-LLS_SF | PHKA2 | S | F | 6 | LLSIISFPA | 2792 | 33.3 | LLSIIFFPA | 2950 | 43.9 |
| PIGN-FLT | PIGN-FLT_P7H | PIGN | P | H | 7 | FLTVFSPFM | 2793 | 11.5 | FLTVFSHFM | 2951 | 25.7 |
| PLXNB1-VLF | PLXNB1-VLF_V1L | PLXNB1 | V | L | 1 | VLFAAFSSA | 2794 | 33.5 | LLFAAFSSA | 2952 | 26 |
| PRSS16-LLL | PRSS16-LLL_L1Q | PRSS16 | L | Q | 1 | LLLVSLWGL | 2795 | 9.4 | QLLVSLWGL | 2953 | 22.9 |
| PTCHD4-HQL | PTCHD4-HQL_G5V | PTCHD4 | G | V | 5 | HQLGGVVEV | 2796 | 49.2 | HQLGVVVEV | 2954 | 54 |
| PXDNL-SIL | PXDNL-SIL_S1F | PXDNL | S | F | 1 | SILDAVQRV | 2797 | 31.4 | FILDAVQRV | 2955 | 5.7 |
| REV3L-KLS | REV3L-KLS_R6H | REV3L | R | H | 6 | KLSEYRNSL | 2798 | 49.7 | KLSEYHNSL | 2956 | 19.7 |
| RRP1B-LLA | RRP1B-LLA_L7F | RRP1B | L | F | 7 | LLADQNLKFI | 2799 | 83.6 | LLADQNFKFI | 2957 | 30.1 |
| RYR3-VLN | RYR3-VLN_E6K | RYR3 | E | K | 6 | VLNYFEPYL | 2800 | 10.2 | VLNYFKPYL | 2958 | 20.4 |
| SCN3A-ALV | SCN3A-ALV_P7S | SCN3A | P | S | 7 | ALVGAIPSI | 2801 | 12.3 | ALVGAISSI | 2959 | 50.4 |
| SEC24A-FLY | SEC24A-FLY_P5L | SEC24A | P | L | 5 | FLYNPLTRV | 2802 | 4.4 | FLYNLLTRV | 2960 | 3.3 |
| SH3RF2-HMV | SH3RF2-HMV_MI | SH3RF2 | M | 1 | 2 | HMVEISTPV | 2803 | 6.4 | HIVEISTPV | 2961 | 34.1 |
| SHROOM2-KLL | SHROOM2-KLL_D6V | SHROOM2 | D | V | 6 | KLLAGDEIV | 2804 | 31.1 | KLLAGVEIV | 2962 | 11.1 |
| SLC15A2-ILG | SLC15A2-ILG_G4E | SLC15A2 | G | E | 4 | ILGGQVVHTV | 2805 | 86.8 | ILGEQVVHTV | 2963 | 49 |
| SLC16A7-AMA | SLC16A7-AMA_P6L | SLC16A7 | P | L | 6 | AMAGSPVFL | 2806 | 19.4 | AMAGSLVFL | 2964 | 8.1 |
| SLC1A2-YMS | SLC1A2-YMS_S3P | SLC1A2 | S | P | 3 | YMSTTIIAA | 2807 | 8.3 | YMPTTIIAA | 2965 | 13.8 |
| SLC2A3-ILV | SLC2A3-ILV_L9M | SLC2A3 | L | M | 9 | ILVAQIFGL | 2808 | 9 | ILVAQIFGM | 2966 | 28 |
| SLC2A4-ILI | SLC2A4-ILI_A4T | SLC2A4 | A | T | 4 | ILIAQVLGL | 2809 | 17.4 | ILITQVLGL | 2967 | 22.6 |
| SLC38A1-RIW | SLC38A1-RIW_W3L | SLC38A1 | W | L | 3 | RIWAALFLGL | 2810 | 70.9 | RILAALFLGL | 2968 | 96.9 |
| SLC39A4-LLG | SLC39A4-LLG_G4S | SLC39A4 | G | S | 4 | LLGGVVTVLL | 2811 | 27.9 | LLGSWTVLL | 2969 | 22.7 |
| SMARCD3-KLF | SMARCD3-KLF_H8Y | SMARCD3 | H | Y | 8 | KLFEFLVHGV | 2812 | 4.4 | KLFEFLVYGV | 2970 | 3.3 |
| SMOX-KLA | SMOX-KLA_KN | SMOX | K | N | 4 | KLAKPLPYT | 2813 | 88.9 | KLANPLPYT | 2971 | 59.8 |
| SNX24-KLS | SNX24-KLS_P6L | SNX24 | P | L | 6 | KLSHQPVLL | 2814 | 85.1 | KLSHQLVLL | 2972 | 25.8 |
| SPOP N147I- | SPOP N147I- | SPOP | N | I | 7 | FLLDEANGL | 2815 | 5.5 | FLLDEAIGL | 2973 | 3.3 |
| FLL | FLL_N7I | | | | | | | | | | |
| SREBF1-YLQ | SREBF1-YLQ_L6M | SREBF1 | L | M | 6 | YLQDSLATT | 2816 | 20 | YLQDSMATT | 2974 | 28.2 |
| SSPN-10 | SSPN-10_S9F | SSPN | S | F | 9 | FLMASISSSL | 2817 | 9.2 | FLMASISSFL | 2975 | 6.3 |
| SSPN-9 | SSPN-9_S9F | SSPN | S | F | 9 | FLMASISSS | 2818 | 21.8 | FLMASISSF | 2976 | 31.7 |
| SSPN-LMA | SSPN-LMA_S8F | SSPN | S | F | 8 | LMASISSSL | 2819 | 22.7 | LMASISSFL | 2977 | 10.5 |
| ST6GALNAC2- | ST6GALNAC2- | ST6GALNAC2 | Y | H | 6 | LLFALYFSA | 2820 | 7.4 | LLFALHFSA | 2978 | 9.6 |
| LLF | LLF_Y6H | | | | | | | | | | |
| STOX1-RLM | STOX1-RLM_M3I | STOX1 | M | I | 3 | RLMKHYPGI | 2821 | 18.5 | RLIKHYPGI | 2979 | 50.4 |
| TAS1R2-FMS | TAS1R2-FMS_A4S | TAS1R2 | A | S | 4 | FMSAYSGVL | 2822 | 25.4 | FMSSYSGVL | 2980 | 28 |
| TBX3-GMG | TBX3-GMG_T8M | TBX3 | T | M | 8 | GMGPLLATV | 2823 | 19.7 | GMGPLLAMV | 2981 | 20.2 |
| TEAD1-SVL | TEAD1-SVL_L9F | TEAD1 | L | F | 9 | SVLENFTILL | 2824 | 182.7 | SVLENFTIFL | 2982 | 84.7 |
| TEAD1-VLE | TEAD1-VLE_L8F | TEAD1 | L | F | 8 | VLENFTILLV | 2825 | 138.5 | VLENFTIFLV | 2983 | 50.6 |
| TEX2-FLM | TEX2-FLM_K8N | TEX2 | K | N | 8 | FLMTLETKM | 2826 | 13.2 | FLMTLETNM | 2984 | 9.3 |
| TMEM127-VTF | TMEM127-VTF_L9V | TMEM127 | L | V | 9 | VTFAVSFYLV | 2827 | 41.4 | VTFAVSFYVV | 2985 | 41.4 |
| TMEM195-ALS | TMEM195-ALS_S3L | TMEM195 | S | L | 3 | ALSQVTLLL | 2828 | 73.6 | ALLQVTLLL | 2986 | 40.6 |
| TP73-YTP | TP73-YTP_P3S | TP73 | P | S | 3 | YTPEHAASV | 2829 | 69 | YTSEHAASV | 2987 | 34.2 |
| TPP2-SLA | TPP2-SLA_WL | TPP2 | W | L | 7 | SLAETFWET | 2830 | 10.3 | SLAETFLET | 2988 | 52 |
| TRIM16-RMA | TRIM16-RMA_R1T | TRIM16 | R | T | 1 | RMAAISNTV | 2831 | 14.3 | TMAAISNTV | 2989 | 15.3 |
| TRIM58-VLA | TRIM58-VLA_V1F | TRIM58 | V | F | 1 | VLASPSVPL | 2832 | 38.5 | FLASPSVPL | 2990 | 5.9 |
| TRIM58-YMV | TRIM58-YMV_V3F | TRIM58 | V | F | 3 | YMVLASPSV | 2833 | 4.8 | YMFLASPSV | 2991 | 2.8 |
| TRPC1-MLL | TRPC1-MLL_Q5H | TRPC1 | Q | H | 5 | MLLKQDVSL | 2834 | 27.6 | MLLKHDVSL | 2992 | 15.4 |
| TRPV3-LLL | TRPV3-LLL_A8V | TRPV3 | A | V | 8 | LLLNMLIAL | 2835 | 8.5 | LLLNMLIVL | 2993 | 17.1 |
| TRPV4-FMI | TRPV4-FMI_A6T | TRPV4 | A | T | 6 | FMIGYASAL | 2836 | 5.2 | FMIGYTSAL | 2994 | 3.8 |
| TRPV4-YLL | TRPV4-YLL_A9T | TRPV4 | A | T | 9 | YLLFMIGYA | 2837 | 10.5 | YLLFMIGYT | 2995 | 31.3 |
| TTLL12-KLP | TTLL12-KLP_N7D | TTLL12 | N | D | 7 | KLPLDINPV | 2838 | 15.7 | KLPLDIDPV | 2996 | 21.4 |
| UNC13A-SQL | UNC13A-SQL_S1F | UNC13A | S | F | 1 | SQLNQSFEI | 2839 | 80 | FQLNQSFEI | 2997 | 8.9 |
| USP28-LII | USP28-LII_C5F | USP28 | C | F | 5 | LIIPCIHLI | 2840 | 32.7 | LIIPFIHLI | 2998 | 24.5 |
| VN1R2-LML | VN1R2-LML_L3F | VN1R2 | L | F | 3 | LMLWASSSI | 2841 | 37.3 | LMFWASSSI | 2999 | 23.1 |
| VN1R5-MII | VN1R5-MII_S7Y | VN1R5 | S | Y | 7 | MIISHLSLI | 2842 | 30.9 | MIISHLYLI | 3000 | 7.9 |
| WDR46-FLT | WDR46-FLT_T3I | WDR46 | T | I | 3 | FLTYLDVSV | 2843 | 6.4 | FLIYLDVSV | 3001 | 4 |
| ZDHHC17-LLL | ZDHHC17-LLL_T41 | ZDHHC17 | T | I | 4 | LLLTFNVSV | 2844 | 5.2 | LLLIFNVSV | 3002 | 14.5 |
| ZDHHC7-SLL | ZDHHC7-SLL_P7L | ZDHHC7 | P | L | 7 | SLLWMNPFV | 2845 | 3.7 | SLLWMNLFV | 3003 | 5.1 |
| ZFP90-FTQ | ZFP90-FTQ_EK | ZFP90 | E | K | 5 | FTQEEWYHV | 2846 | 23 | FTQEKWYHV | 3004 | 26.8 |
| ZNF827-NLF | ZNF827-NLF_54I | ZNF827 | S | I | 4 | NLFSQDISV | 2847 | 16 | NLFIQDISV | 3005 | 46.4 |

TABLE 9
TetTCR summary for experiment 5.

| | Sorted | | | | | | | | | | | | | | SEQ ID |
|---|---|---|---|---|---|---|---|---|---|---|---|---|---|---|---|
| Cell | Popu- | Detected Peptide by MID Count | | | | | TCRα, 1 | | TCRα, 2 | | TCRβ | | TCRβ | | NO (L |
| Name | lation | Rank 1 | Rank 2 | Rank 3 | Rank 4 | Rank 5 | TRAV | CDR3α | TRAV | CDR3α | TRBV | CDR3β | TRBV | CDR3β | to R) |
| SA1 | Clone | HCV- | HCV- | 0 | 0 | 0 | 38-2/ | CAYRSPPSS | | | 28*01 | CASSFLGTG | | | 3006 |
| | | KLV(PE) | KLV(APC) | | | | DV8*01 | EKLVF | | | | LNEQYF | | | 3490 |
| SB1 | Clone | HCV- | HCV- | 0 | 0 | 0 | 38-2/ | CAYRSPPSS | | | 28*01 | CASSFLGTG | | | 3006 |
| | | KLV(APC) | KLV(PE) | | | | DV8*01 | EKLVF | | | | LNEQYF | | | 3490 |
| SC1 | Clone | HCV- | HCV- | 0 | 0 | 0 | 38-2/ | CAYRSPPSS | | | 28*01 | CASSFLGTG | | | 3006 |
| | | KLV(APC) | KLV(PE) | | | | DV8*01 | EKLVF | | | | LNEQYF | | | 3490 |
| SD1 | Clone | 0 | 0 | 0 | 0 | 0 | | | | | | | | | |
| SE1 | Clone | HCV- | HCV- | 0 | 0 | 0 | 38-2/ | CAYRSPPSS | | | 28*01 | CASSFLGTG | | | 3006 |
| | | KLV(APC) | KLV(PE) | | | | DV8*01 | EKLVF | | | | LNEQYF | | | 3490 |
| SF1 | Clone | HCV- | HCV- | 0 | 0 | 0 | | | | | | | | | |
| | | KLV(APC) | KLV(PE) | | | | | | | | | | | | |
| SG1 | Clone | HCV- | HCV- | 0 | 0 | 0 | 38-2/ | CAYRSPPSS | | | 28*01 | CASSFLGTG | | | 3006 |
| | | KLV(APC) | KLV(PE) | | | | DV8*01 | EKLVF | | | | LNEQYF | | | 3490 |
| SH1 | Clone | HCV- | HCV- | 0 | 0 | 0 | 38-2/ | CAYRSPPSS | | | 28*01 | CASSFLGTG | | | 3006 |
| | | KLV(APC) | KLV(PE) | | | | DV8*01 | EKLVF | | | | LNEQYF | | | 3490 |
| GA10 | Neo⁺WT⁺ | FNDC3B- | FNDC3B- | 0 | 0 | 0 | 8- | CAVGAEDSN | | | 6-2*01, | CASSYSWGE | | | 3007 |
| | | VVL_L3M | VVL | | | | 3*01 | YQLIW | | | 6-3*01 | QFF | | | 3491 |
| GA12 | Neo⁺WT⁺ | OR6F1- | OR6F1- | 0 | 0 | 0 | | | | | 6-2*01, | CASTHWERV | | | 3492 |
| | | VLN | VLN_T8M | | | | | | | | 6-3*01 | DEQFF | | | |
| GA6 | Neo⁺WT⁺ | OR14C36- | OR14C36- | IL17RA- | 0 | 0 | 17*01 | CATDVNNDM | | | 6-5*01 | CASSYGVNT | | | 3008 |
| | | FML_V6L | FML | FIT_TM | | | | RF | | | | EAFF | | | 3493 |
| GB1 | Neo⁺WT⁺ | TTLL12- | TTLL12- | GP100- | 0 | 0 | 24*01 | CASFMDSNY | | | 10-3*01 | CAISRGDTE | | | 3009 |
| | | KLP_N7D | KLP | ALL | | | | QLIW | | | | AFF | | | 3494 |
| GB2 | Neo⁺WT⁺ | ME1- | ME1-FLD | 0 | 0 | 0 | 14/ | CAMRASLQG | | | 15*01 | CATSAKTRL | | | 3010 |
| | | FLD_A8G | | | | | DV4*01 | AQKLVF | | | | NTEAFF | | | 3495 |
| GB4 | Neo⁺WT⁺ | OR14C36- | 0 | 0 | 0 | 0 | 17*01 | CATDAQFLR | | | | | | | 3011 |
| | | FML_V6L | | | | | | SGAGSYQLT | | | | | | | |
| | | | | | | | | F | | | | | | | |
| GB8 | Neo⁺WT⁺ | 0 | 0 | 0 | 0 | 0 | 9-2*01 | CALWGTYKY | | | 13*01 | CASSKGQGA | | | 3012 |
| | | | | | | | | IF | | | | NYGYTF | | | 3496 |
| GC12 | Neo⁺WT⁺ | RYR3- | RYR3-VLN | TAS1R2- | OR10A3- | 0 | 8-3*01 | CAVGGEKLT | | | 5-1*01 | CASSLIDSPY | | | 3013 |
| | | VLN_E6K | | FMS | ILI | | | F | | | | EQYF | | | 3497 |
| GC5 | Neo⁺WT⁺ | FNDC3B- | FNDC3B- | 0 | 0 | 0 | 29/ | CAASATGGT | | | | | | | 3014 |
| | | VVL_L3M | VVL | | | | DV5*01 | SYGKLTF | | | | | | | |
| GD1 | Neo⁺WT⁺ | DHX33- | DHX33- | 0 | 0 | 0 | 12-2*01 | CASEVGGYA | | | | | | | 3015 |
| | | LLA | LLA_M4I | | | | | LNF | | | | | | | |
| GD2 | Neo⁺WT⁺ | 0 | 0 | 0 | 0 | 0 | | | | | | | | | |
| GD6 | Neo⁺WT⁺ | IGF1- | 0 | 0 | 0 | 0 | | | | | | | | | |
| | | TMS_S4F | | | | | | | | | | | | | |
| GD8 | Neo⁺WT⁺ | HAUS3- | HAUS3- | 0 | 0 | 0 | 24*01 | CAPHSGYST | | | 28*01 | CASSLGPNS | | | 3016 |
| | | ILN_T7A | ILN | | | | | LTF | | | | PLHF | | | 3498 |
| GE1 | Neo⁺WT⁺ | DHX33- | 0 | 0 | 0 | 0 | 12-2*01 | CAVIGTDKLI | | | 2*01 | CASGSYEQY | | | 3017 |
| | | LLA_M4I | | | | | | F | | | | F | | | 3499 |
| GE11 | Neo⁺WT⁺ | FNDC3B- | FNDC3B- | 0 | 0 | 0 | 29/ | CAASHGSSN | | | | | | | 3018 |
| | | VVL_L3M | VVL | | | | DV5*01 | TGKLIF | | | | | | | |
| GE2 | Neo⁺WT⁺ | 0 | 0 | 0 | 0 | 0 | | | | | | | | | |
| GE3 | Neo⁺WT⁺ | NSDHL- | 0 | 0 | 0 | 0 | 24*01 | CAFSGNTPL | | | | | | | 3019 |
| | | ILT_A9V | | | | | | VF | | | | | | | |
| GE9 | Neo⁺WT⁺ | HTR1F- | HTR1F-9 | GLRA1- | HTR1F- | 0 | 9-2*01 | CALSDRGGG | | | 6-5*01 | CASSSQTGP | | | 3020 |
| | | 9_V1M | | LIF_F6L | LVM_V2M | | | ADGLTF | | | | YSNQPQHF | | | 3500 |
| GF1 | Neo⁺WT⁺ | VN1R2- | MPV17- | VN1R2- | 0 | 0 | 12-2*01 | CAVGGDSSY | | | | | | | 3021 |
| | | LML_L3F | YLW_A5P | LML | | | | KLIF | | | | | | | |
| GF12 | Neo⁺WT⁺ | PHKA2- | PHKA2- | 0 | 0 | 0 | | | | | 6-5*01 | CASRDSVGG | | | 3501 |
| | | LLS_SF | LLS | | | | | | | | | GEGYTF | | | |
| GF2 | Neo⁺WT⁺ | SLC1A2- | 0 | 0 | 0 | 0 | 12-2*01 | CAAPPDSSY | | | | | | | 3022 |
| | | YMS_S3P | | | | | | KLIF | | | | | | | |
| GF3 | Neo⁺WT⁺ | GABRG3- | 0 | 0 | 0 | 0 | | | | | | | | | |
| | | TAM_L5I | | | | | | | | | | | | | |
| GF7 | Neo⁺WT⁺ | TRPV4- | TRPV4- | 0 | 0 | 0 | | | | | 27*01 | CASSVTGRW | | | 3502 |
| | | YLL_A9T | YLL | | | | | | | | | VPLHF | | | |
| GG5 | Neo⁺WT⁺ | 0 | 0 | 0 | 0 | 0 | | | | | | | | | |
| GH11 | Neo⁺WT⁺ | APBB2- | APBB2- | 0 | 0 | 0 | 12-2*01 | CAVTPTDSW | | | 13*01 | CASSQNGSE | | | 3025 |
| | | VQY | VQY_L7F | | | | | GKLQF | | | | AAYSNQPQH | | | 3503 |
| | | | | | | | | | | | | F | | | |
| GH2 | Neo⁺WT⁺ | CNKSR1- | CNKSR1- | 0 | 0 | 0 | | | | | | | | | |
| | | SLA_A9V | SLA | | | | | | | | | | | | |
| GH4 | Neo⁺WT⁺ | DOCK7- | DOCK7- | 0 | 0 | 0 | 21*01 | CAVRPLNTG | | | | | | | 3024 |
| | | FLN_M9L | FLN | | | | | TASKLTF | | | | | | | |
| GH5 | Neo⁺WT⁺ | OR6F1- | OR6F1- | 0 | 0 | 0 | 41*01 | CAVEGSRLT | | | | | | | 3025 |
| | | VLN_T8M | VLN | | | | | F | | | | | | | |
| GH6 | Neo⁺WT⁺ | DHX33- | DHX33- | KCNB2- | 0 | 0 | | | | | | | | | |
| | | LLA_M4I | LLA | LLA_P6T | | | | | | | | | | | |
| GH7 | Neo⁺WT⁺ | HTR1F- | HTR1F- | 0 | 0 | 0 | | | | | | | | | |
| | | 10_V1M | 10 | | | | | | | | | | | | |
| GH9 | Neo⁺WT⁺ | IL17RA- | 0 | 0 | 0 | 0 | | | | | | | | | |
| | | FIT_TM | | | | | | | | | | | | | |
| IA10 | Neo⁺WT⁺ | NSDHL- | NSDHL- | 0 | 0 | 0 | | | | | 20-1*01 | CSATGQNYE | | | 3504 |
| | | ILT_A9V | ILT | | | | | | | | | QYF | | | |
| IA4 | Neo⁺WT⁺ | DOCK7- | DOCK7- | 0 | 0 | 0 | 8-1*01 | CAVNAPTGF | | | 11-2*01 | CASSIGTVN | | | 3026 |
| | | FLN_M9L | FLN | | | | | QKLVF | | | | RGPNTEAFF | | | 3505 |
| IA5 | Neo⁺WT⁺ | 0 | 0 | 0 | 0 | 0 | 38-1*01 | CAFRQGGSE | | | 19*01 | CASSWQGS | | | 3027 |
| | | | | | | | | KLVF | | | | NIQYF | | | 3506 |
| IA9 | Neo⁺WT⁺ | OR5M3- | OR5M3- | 0 | 0 | 0 | 12-2*01 | CAVREYSGG | | | 5-6*01 | CASSPITNTG | | | 3028 |
| | | KMV | KMV_T8N | | | | | GADGLTF | | | | ELFF | | | 3507 |
| IB1 | Neo⁺WT⁺ | CLCN4- | CLCN4- | 0 | 0 | 0 | 19*01 | CALSEAYNN | | | 20-1*01 | CSATLDRNY | | | 3029 |
| | | LLA_G8V | LLA | | | | | NDMRF | | | | GYTF | | | 3508 |
| IB11 | Neo⁺WT⁺ | HTR1F- | HTR1F-9 | 0 | 0 | 0 | | | | | | | | | |
| | | 9_V1M | | | | | | | | | | | | | |
| IB4 | Neo⁺WT⁺ | CHD8- | CHD8- | 0 | 0 | 0 | 12-1*01 | CVVNVDNAG | | | 7-9*01 | CASSLETGG | | | 3030 |
| | | KLN_P7A | KLN | | | | | NMLTF | | | | WETQYF | | | 3509 |
| IB6 | Neo⁺WT⁺ | TRPC1- | TRPC1- | 0 | 0 | 0 | | | | | 12-3*01, | CASSLNMNT | | | 3510 |
| | | MLL_Q5H | MLL | | | | | | | | 12-4*01 | EAFF | | | |
| IC10 | Neo⁺WT⁺ | IGF1- | HBZ-KLS | 0 | 0 | 0 | | | | | 5-1*01 | CASSIDRTV | | | 3511 |
| | | TMS_S4F | | | | | | | | | | GNTIYF | | | |
| IC6 | Neo⁺WT⁺ | GALC- | GALC- | DRAM1- | 0 | 0 | | | | | | | | | |
| | | YVV_V3L | YVV | FII_I3F | | | | | | | | | | | |
| ID7 | Neo⁺WT⁺ | GABRG3- | GABRG3- | 0 | 0 | 0 | 29/ | CAARLYGGS | | | 20-1*01 | CSARDWGY | | | 3031 |
| | | TAM_L5I | TAM | | | | DV5*01 | QGNLIF | | | | EQYF | | | 3512 |
| ID9 | Neo⁺WT⁺ | HAUS3- | HAUS3- | 0 | 0 | 0 | | | | | | | | | |
| | | ILN_T7A | ILN | | | | | | | | | | | | |
| IE1 | Neo⁺WT⁺ | OR6F1- | OR6F1- | 0 | 0 | 0 | | | | | | | | | |
| | | VLN_T8M | VLN | | | | | | | | | | | | |
| IE2 | Neo⁺WT⁺ | 0 | 0 | 0 | 0 | 0 | | | | | | | | | |
| IE3 | Neo⁺WT⁺ | GABRG3- | GABRG3- | 0 | 0 | 0 | | | | | | | | | |
| | | TAM_L5I | TAM | | | | | | | | | | | | |
| IE7 | Neo⁺WT⁺ | 0 | 0 | 0 | 0 | 0 | 12-2*01 | CAVNEGGTS | | | 27*01 | CASSFGSGG | | | 3032 |
| | | | | | | | | YGKLTF | | | | ALYF | | | 3513 |
| IF3 | Neo⁺WT⁺ | TRPC1- | 0 | 0 | 0 | 0 | | | | | | | | | |
| | | MLL_Q5H | | | | | | | | | | | | | |
| IF4 | Neo⁺WT⁺ | HTR1F- | 0 | 0 | 0 | 0 | | | | | | | | | |
| | | 10_V1M | | | | | | | | | | | | | |
| IF6 | Neo⁺WT⁺ | TRIM16- | 0 | 0 | 0 | 0 | 8-1*01 | CAVFTGGGN | 12-2*01 | CAVRSGA | 7-2*01 | CASSFLLYN | | | 3033 |
| | | RMA_R1T | | | | | | KLTF | | GSYQLTF | | EQFF | | | 3450 |
| | | | | | | | | | | | | | | | 3514 |
| IF8 | Neo⁺WT⁺ | IL17RA- | IL17RA- | 0 | 0 | 0 | 12-3*01 | CAISMDTGR | | | 6-1*01 | CASSEMDGS | | | 3034 |
| | | FIT_TM | FIT | | | | | RALTF | | | | NQPQHF | | | 3515 |
| IF9 | Neo⁺WT⁺ | BAIAP3- | BAIAP3- | 0 | 0 | 0 | 12-2*01 | CAVRLVGGT | | | 29-1*01 | CSVRLTDYN | | | 3035 |
| | | ILN_V6I | ILN | | | | | SYGKLTF | | | | EQFF | | | 3516 |
| IG2 | Neo⁺WT⁺ | HAUS3- | 0 | 0 | 0 | 0 | | | | | | | | | |
| | | ILN_T7A | | | | | | | | | | | | | |
| IG8 | Neo⁺WT⁺ | SHROOM2- | SHROOM2- | 0 | 0 | 0 | 17*01 | CATLGDNDM | | | | | | | 3036 |
| | | KLL D6V | KLL | | | | | RF | | | | | | | |
| IG9 | Neo⁺WT⁺ | OR5M3- | CELSR1- | 0 | 0 | 0 | 14/ | CAMREGWG | | | 9*01 | CASSGSGAS | | | 3037 |
| | | KMV_T8N | YLF_F3L | | | | DV4*01 | DMRF | | | | TDTQYF | | | 3517 |
| IH12 | Neo⁺WT⁺ | 0 | 0 | 0 | 0 | 0 | | | | | | | | | |
| IH3 | Neo⁺WT⁺ | 0 | 0 | 0 | 0 | 0 | 19*01 | CALSGFGMD | | | | | | | 3038 |
| | | | | | | | | SSYKLIF | | | | | | | |
| IH7 | Neo⁺WT⁺ | GALC- | GALC- | 0 | 0 | 0 | 27*01 | CAGIGAGSY | | | | | | | 3039 |
| | | YVV_V3L | YVV | | | | | QLTF | | | | | | | |
| IH8 | Neo⁺WT⁺ | SMOX- | AKAP13- | 0 | 0 | 0 | 24*01 | CAFLMDSSY | | | 27*01 | CASSLGPGG | | | 3040 |
| | | KLA_KN | KLM | | | | | KLIF | | | | ASYTF | | | 3518 |
| JA12 | Neo⁺WT⁺ | HTR1F- | HTR1F-9 | 0 | 0 | 0 | | | | | 25-1*01 | CASSETSLFT | | | 3519 |
| | | 9_V1M | | | | | | | | | | HGYTF | | | |
| JA2 | Neo⁺WT⁺ | SLC15A2- | HAUS3- | 0 | 0 | 0 | 24*01 | CAFIGYGGS | 29/ | CASHGSS | 30*01 | CAWSSSVNT | | | 3041 |
| | | ILG | ILN_T7A | | | | | QGNLIF | DV5*01 | NTGKLIF | | EAFF | | | 3451 |
| | | | | | | | | | | | | | | | 3520 |
| JA4 | Neo⁺WT⁺ | FNDC3B- | FNDC3B- | 0 | 0 | 0 | 20*01 | CAVLTSGYS | | | 13*01 | CASSPMTGA | | | 3042 |
| | | VVL_L3M | VVL | | | | | TLTF | | | | EQFF | | | 3521 |
| JA6 | Neo⁺WT⁺ | HTR1F- | SEC24A- | SEC24A- | CNKSR1- | 0 | 24*01 | CAFIIQGAQ | | | 7-6*01 | CASSLGGLV | | | 3043 |
| | | 10_V1M | FLY | FLY_P5L | SLA_A9V | | | KLVF | | | | YNEQFF | | | 3522 |
| JA7 | Neo⁺WT⁺ | NSDHL- | 0 | 0 | 0 | 0 | | | | | | | | | |
| | | ILT_A9V | | | | | | | | | | | | | |
| JB1 | Neo⁺WT⁺ | 0 | 0 | 0 | 0 | 0 | 21*01 | CAVNSGYST | | | 27*01 | CASSFSGGN | | | 3044 |
| | | | | | | | | LTF | | | | EQFF | | | 3523 |
| JC12 | Neo⁺WT⁺ | 0 | 0 | 0 | 0 | 0 | | | | | 19*01 | CASTSGAYN | | | 3524 |
| | | | | | | | | | | | | EQFF | | | |
| JD11 | Neo⁺WT⁺ | HTR1F- | HTR1F-9 | DOLPP1- | 0 | 0 | 23/ | CAATEGGHN | | | 6-5*01 | CASSYQTGP | | | 3045 |
| | | 9_V1M | | GLM | | | DV6*01 | YGQNFVF | | | | YSNQPQHF | | | 3525 |
| JD3 | Neo⁺WT⁺ | HCV- | HCV- | 0 | 0 | 0 | | | | | | | | | |
| | | KLV(APC) | KLV(PE) | | | | | | | | | | | | |
| JD4 | Neo⁺WT⁺ | KCNB2- | 0 | 0 | 0 | 0 | 26-1*01 | CIVSPGGYQ | | | 27*01 | CASSWVGGA | | | 3046 |
| | | LLA_P6T | | | | | | KVTF | | | | DTQYF | | | 3526 |
| JE12 | Neo⁺WT⁺ | SLC16A7- | HTR1F- | 0 | 0 | 0 | 1-2*01 | CAVNGGDKI | | | 4-1*01 | CASSQDLGT | | | 3047 |
| | | AMA_P6L | 9_V1M | | | | | IF | | | | GNTIYF | | | 3527 |
| JE2 | Neo⁺WT⁺ | GALC- | 0 | 0 | 0 | 0 | | | | | | | | | |
| | | YVV_V3L | | | | | | | | | | | | | |
| JE3 | Neo⁺WT⁺ | 0 | 0 | 0 | 0 | 0 | 14/ | CAMRERGSY | | | | | | | 3048 |
| | | | | | | | DV4*01 | AGGTSYGKL | | | | | | | |
| | | | | | | | | TF | | | | | | | |
| JE4 | Neo⁺WT⁺ | CNKSR1- | 0 | 0 | 0 | 0 | 12-2*01 | CAVNKANDY | | | 20-1*01 | CSASDSLTI | | | 3049 |
| | | SLA_A9V | | | | | | KLSF | | | | SGFF | | | 3528 |
| JE7 | Neo⁺WT⁺ | 0 | 0 | 0 | 0 | 0 | 12-2*01 | CAVTADGQK | | | 5-5*01 | CASSLLGQT | | | 3050 |
| | | | | | | | | LLF | | | | NYGYTF | | | 3529 |
| JE8 | Neo⁺WT⁺ | NSDHL- | NSDHL- | 0 | 0 | 0 | 3*01 | CAVRDDNNN | | | 2*01 | CASSEGQGR | | | 3051 |
| | | ILT_A9V | ILT | | | | | DMRF | | | | WYEQYF | | | 3530 |
| JE9 | Neo⁺WT⁺ | NSDHL- | 0 | 0 | 0 | 0 | 19*01 | CALSEANTG | | | 9*01 | CASSVGSTE | | | 3052 |
| | | ILT_A9V | | | | | | GFKTIF | | | | AFF | | | 3531 |
| JF11 | Neo⁺WT⁺ | OR5M3- | OR5M3- | 0 | 0 | 0 | 14/ | CAMREGDRN | | | 4-2*01 | CASSPWEMN | | | 3053 |
| | | KMV | KMV_T8N | | | | DV4*01 | QFYF | | | | TEAFF | | | 3533 |
| JF12 | Neo⁺WT⁺ | VN1R5- | 0 | 0 | 0 | 0 | 14/ | CAMREAPEN | | | 20-1*01 | CSASVSGGP | | | 3055 |
| | | MII_S7Y | | | | | DV4*01 | GGTSYGKLT | | | | LDTQYF | | | 3533 |
| | | | | | | | | F | | | | | | | |
| JF6 | Neo⁺WT⁺ | BAIAP3- | BAIAP3- | 0 | 0 | 0 | 20*01 | CAVRSNDYK | | | 28*01 | CASSLGPME | | | 3055 |
| | | ILN | ILN_V6I | | | | | LSF | | | | ENIQYF | | | 3534 |
| JG8 | Neo⁺WT⁺ | HTR1F-10 | C17orf75- | C17orf75- | 0 | 0 | 10*01 | CVVRGGYNK | | | 5-4*01 | CASSSDRGE | | | 3056 |
| | | | ALS_V7A | ALS | | | | LIF | | | | QFF | | | 3535 |
| JH1 | Neo⁺WT⁺ | OR6F1- | C15orf32- | ZDHHC7- | 0 | 0 | | | | | | | | | |
| | | VLN_T8M | MLS_G9R | SLL | | | | | | | | | | | |
| JH6 | Neo⁺WT⁺ | 0 | 0 | 0 | 0 | 0 | | | | | | | | | |
| JH9 | Neo⁺WT⁺ | 0 | 0 | 0 | 0 | 0 | 5*01 | CAETGAGN | 12-2*01 | CAGDSWG | | | | | 3057 |
| | | | | | | | | MLTF | | KLQF | | | | | 3452 |
| KA1 | Neo⁺WT⁺ | HAUS3- | VN1R2- | 0 | 0 | 0 | | | | | | | | | |
| | | ILN_T7A | LML_L3F | | | | | | | | | | | | |
| KA10 | Neo⁺WT⁺ | ST6GALNAC2- | ST6GALNAC2- | KCNB2- | PHKA2- | 0 | 12-3*01 | CAFYDYKLS | | | 6-1*01 | CASSEVEGP | | | 3058 |
| | | LLF_Y6H | LLF | LLA_P6T | LLS_SF | | | F | | | | GELFF | | | 3536 |
| KA11 | Neo⁺WT⁺ | C3orf58- | C3orf58- | 0 | 0 | 0 | | | | | 27*01 | CASSLSGFG | | | 3537 |
| | | LMV | LMV_L4P | | | | | | | | | NTIYF | | | |
| KA2 | Neo⁺WT⁺ | TRIM58- | TRIM58- | 0 | 0 | 0 | 19*01 | CALSDPYSS | | | 14*01 | CASSQGGQD | | | 3059 |
| | | VLA | VLA_V1F | | | | | ASKIIF | | | | GHGTTNEKL | | | 3538 |
| | | | | | | | | | | | | FF | | | |
| KA6 | Neo⁺WT⁺ | IGF1- | IGF1-TMS | 0 | 0 | 0 | 14/ | CAMREGQD | | | 11-2*01 | CASSLGGGG | | | 3060 |
| | | TMS_S4F | | | | | DV4*01 | ARLMF | | | | PQETQYF | | | 3539 |
| KB12 | Neo⁺WT⁺ | PXDNL- | PXDNL- | 0 | 0 | 0 | 38-2/ | CARPEAGN | | | 10-2*01 | CATSRTDIS | | | 3061 |
| | | SIL_S1F | SIL | | | | DV8*01 | MLTF | | | | YEQYF | | | 3540 |
| KB3 | Neo⁺WT⁺ | TBX3- | TBX3- | 0 | 0 | 0 | | | | | 6-5*01 | CASSYYGTT | | | 3541 |
| | | GMG_T8M | GMG | | | | | | | | | DEQYF | | | |
| | 3062 | | | | | | | | | | | | | | |
| KB4 | Neo⁺WT⁺ | CNKSR1- | NOS1-FID | CNKSR1- | 0 | 0 | 17*01 | CATDEANFG | 17*01 | CARPPDD | 4-2*01 | CASSLGPSL | | | 3453 |
| | | SLA_A9V | | SLA | | | | NEKLTF | | YKLSF | | YEQYF | | | 3542 |
| KB7 | Neo⁺WT⁺ | MRM1- | 0 | 0 | 0 | 0 | 12-2*01 | CAVREGFKTI | | | 11-2*01 | CASSWGSSP | | | 3063 |
| | | 9_T6P | | | | | | F | | | | AETQYF | | | 3543 |
| KC10 | Neo⁺WT⁺ | DRAM1- | OR1G1- | 0 | 0 | 0 | | | | | 18*01 | CASSDQGAL | | | 3544 |
| | | FII_I3F | FLF | | | | | | | | | SSYEQYF | | | |
| KC12 | Neo⁺WT⁺ | RYR3-VLN | RYR3- | 0 | 0 | 0 | 12-1*01 | CGRTDSWG | | | 6-1*01 | CASSRIANN | | | 3064 |
| | | | VLN_E6K | | | | | KLQF | | | | NNEQFF | | | 3545 |
| KD1 | Neo⁺WT⁺ | OR5M3- | OR5M3- | 0 | 0 | 0 | 38-2/ | CAYRKENND | | | | | | | 3065 |
| | | KMV | KMV_T8N | | | | DV8*01 | MRF | | | | | | | |
| KD10 | Neo⁺WT⁺ | HERC1- | HERC1- | 0 | 0 | 0 | | | | | 29-1*01 | CSVPVFGRG | | | 3546 |
| | | SLL_PS | SLL | | | | | | | | | TGELFF | | | |
| KD12 | Neo⁺WT⁺ | HTR1F- | NBPF24- | SLC1A2- | 0 | 0 | | | | | 5-1*01 | CASSLWGTY | | | 3547 |
| | | 9_V1M | LLD_E6G | YMS_S3P | | | | | | | | NEQFF | | | |
| KD3 | Neo⁺WT⁺ | TRPV3- | HTR1F-10 | 0 | 0 | 0 | | | | | | | | | |
| | | LLL_A8V | | | | | | | | | | | | | |
| KD5 | Neo⁺WT⁺ | HTR1F- | 0 | 0 | 0 | 0 | | | | | 4-1*01 | CASSQADHY | | | 3548 |
| | | 10_V1M | | | | | | | | | | EQYF | | | |
| KD8 | Neo⁺WT⁺ | 0 | 0 | 0 | 0 | 0 | 12-2*01 | CAVIAGGFK | | | 27*01 | CASSLFNEQ | | | 3066 |
| | | | | | | | | TIF | | | | FF | | | 3549 |
| KD9 | Neo⁺WT⁺ | BTBD1- | BTBD1- | 0 | 0 | 0 | 8-3*01 | CAVGRRNS | | | | | | | 3067 |
| | | FML_LI | FML | | | | | GGYQKVTF | | | | | | | |
| KE12 | Neo⁺WT⁺ | OR5M3- | GABRG3- | 0 | 0 | 0 | 17*01 | CATFPMKTS | | | | | | | 3068 |
| | | KMV | TAM_L5I | | | | | YDKVIF | | | | | | | |
| KE3 | Neo⁺WT⁺ | OVOL1- | OVOL1- | 0 | 0 | 0 | | | | | | | | | |
| | | SLL_L9V | SLL | | | | | | | | | | | | |
| KE7 | Neo⁺WT⁺ | NSDHL- | 0 | 0 | 0 | 0 | | | | | | | | | |
| | | ILT_A9V | | | | | | | | | | | | | |
| KE8 | Neo⁺WT⁺ | HBZ-KLS | HBZ- | 0 | 0 | 0 | 1-2*01 | CAVGLGGGY | | | 27*01 | CASSFGGAS | | | 3069 |
| | | | KLS_A7T | | | | | NKLIF | | | | EAFF | | | 3550 |
| KE9 | Neo⁺WT⁺ | 0 | 0 | 0 | 0 | 0 | 12-2*01 | CAVNEERTD | | | 9*01 | CASSVGNTE | | | 3070 |
| | | | | | | | | KLIF | | | | AFF | | | 3551 |
| KF1 | Neo⁺WT⁺ | HBZ-KLS | HBZ- | 0 | 0 | 0 | 1-2*01 | CAVASGGYN | | | | | | | 3071 |
| | | | KLS_A7T | | | | | KLIF | | | | | | | |
| KF12 | Neo⁺WT⁺ | HAUS3- | 0 | 0 | 0 | 0 | | | | | | | | | |
| | | ILN_T7A | | | | | | | | | | | | | |
| KF2 | Neo⁺WT⁺ | BAIAP3- | 0 | 0 | 0 | 0 | | | | | | | | | |
| | | ILN_V6I | | | | | | | | | | | | | |
| KF4 | Neo⁺WT⁺ | HTR1F- | 0 | 0 | 0 | 0 | | | | | 6-5*01 | CASSPILTYE | | | 3552 |
| | | 9_V1M | | | | | | | | | | QYF | | | |
| KG4 | Neo⁺WT⁺ | TBX3- | 0 | 0 | 0 | 0 | 38-2/ | CAYRSGEYG | | | 19*01 | CASSMAGSS | | | 3072 |
| | | GMG_T8M | | | | | DV8*01 | NKLVF | | | | YEQYF | | | 3553 |
| KG7 | Neo⁺WT⁺ | 0 | 0 | 0 | 0 | 0 | | | | | | | | | |
| KG9 | Neo⁺WT⁺ | TRIM16- | GLRA1- | 0 | 0 | 0 | 25*01 | CAGNDYKLS | | | 12-3*01, | CASSLAQSD | | | 3073 |
| | | RMA | LIF_F6L | | | | | F | | | 12-4*01 | SLAFF | | | 3554 |
| KH2 | Neo⁺WT⁺ | HTR1F- | 0 | 0 | 0 | 0 | | | | | 27*01 | CASSLQGSD | | | 3555 |
| | | LVM_V2M | | | | | | | | | | NEQFF | | | |
| KH9 | Neo⁺WT⁺ | BCL9L- | 0 | 0 | 0 | 0 | 19*01 | CALSDPNDY | | | | | | | 3074 |
| | | FVY_T6I | | | | | | KLSF | | | | | | | |
| LA1 | Neo⁺WT⁺ | 0 | 0 | 0 | 0 | 0 | | | | | 20-1*01 | CSARDLTVA | | | 3556 |
| | | | | | | | | | | | | ETQYF | | | |
| LA2 | Neo⁺WT⁺ | HAUS3- | VN1R5- | HAUS3- | MAR11- | 0 | 12-2*01 | CAVYSGGGA | | | 6-5*01 | CASSSGGA | | | 3075 |
| | | ILN_T7A | MII_S7Y | ILN | 9_F1L | | | DGLTF | | | | WYTF | | | 3557 |
| LA5 | Neo⁺WT⁺ | BAIAP3- | PELP1- | MAR11- | HAUS3- | USP28- | | | | | 2*01 | CASSPRGVG | | | 3558 |
| | | ILN_V6I | LVL_L3F | 9_F1L | ILN | LII_C5F | | | | | | TEAFF | | | |
| LA7 | Neo⁺WT⁺ | NSDHL- | NSDHL- | 0 | 0 | 0 | 14/ | CAMREGLSN | | | 4-1*01 | CASSPSSGG | | | 3076 |
| | | ILT_A9V | ILT | | | | DV4*01 | YGGSQGNLI | | | | ITDTQYF | | | 3559 |
| | | | | | | | | F | | | | | | | |
| LB10 | Neo⁺WT⁺ | VN1R2- | 0 | 0 | 0 | 0 | | | | | 6-1*01 | CASSEQGGE | | | 3560 |
| | | LML_L3F | | | | | | | | | | RRNTEAFF | | | |
| LB12 | Neo⁺WT⁺ | RYR3- | ITIH6- | ITIH6- | 0 | 0 | 29/ | CAASGGGA | 38-1*01 | CAFMKQS | | | | | 3077 |
| | | VLN_E6K | RLG_G3V | RLG | | | DV5*01 | QKLVF | | YRDDKIIF | | | | | 3454 |
| LB3 | Neo⁺WT⁺ | 0 | 0 | 0 | 0 | 0 | 40*01 | CLLGGSNYK | | | | | | | 3078 |
| | | | | | | | | LTF | | | | | | | |
| LB4 | Neo⁺WT⁺ | ITIH6- | 0 | 0 | 0 | 0 | 14/ | CAMRAGYNT | | | 4-1*01 | CASSQGWG | | | 3079 |
| | | RLG_G3V | | | | | DV4*01 | DKLIF | | | | VETQYF | | | 3561 |
| LC1 | Neo⁺WT⁺ | PHKA2- | PHKA2- | 0 | 0 | 0 | 12-2*01 | CAVGSQGNL | 12-1 | VLFRMLTF | 6-5*01 | CASSYSTGG | | | 3080 |
| | | LLS_SF | LLS | | | | | IF | | | | TDTQYF | | | 3455 |
| | | | | | | | | | | | | | | | 3562 |
| LC11 | Neo⁺WT⁺ | 0 | 0 | 0 | 0 | 0 | 21*01 | CAVSGYSTL | | | 19*01 | CASSRTQGY | | | 3081 |
| | | | | | | | | TF | | | | SNQPQHF | | | 3563 |
| LC3 | Neo⁺WT⁺ | BAIAP3- | BAIAP3- | 0 | 0 | 0 | 5*01 | CAEIPRSPM | | | 28*01 | CASSIFTRRG | | | 3082 |
| | | ILN_V6I | ILN | | | | | FSGGYNKLI | | | | YEQYF | | | 3564 |
| | | | | | | | | F | | | | | | | |
| LC5 | Neo⁺WT⁺ | TRIM58- | OR5K2- | TRIM58- | 0 | 0 | 5*01 | CAETLYNQG | | | 9*01 | CASSGRQGI | | | 3083 |
| | | YMV_V3F | YIF | YMV | | | | GKLIF | | | | DTEAFF | | | 3565 |
| LD10 | Neo⁺WT⁺ | 0 | 0 | 0 | 0 | 0 | 8-2*01 | CVVERGSTL | | | 30*01 | CAWIDFLGQ | | | 3084 |
| | | | | | | | | GRLYF | | | | MNTEAFF | | | 3566 |
| LD11 | Neo⁺WT⁺ | ST6GALNAC2- | ST6GALNAC2- | MAR11- | 0 | 0 | 12-3*01 | CAMGDARL | | | 15*01 | CATSGTGGT | | | 3085 |
| | | LLF_Y6H | LLF | 9_F1L | | | | MF | | | | GELFF | | | 3567 |
| LD4 | Neo⁺WT⁺ | PIGN- | 0 | 0 | 0 | 0 | 12-2*01 | CAVLNSGGY | | | 6-2*01, | CASSLSYEQ | | | 3086 |
| | | FLT_P7H | | | | | | QKVTF | | | 6-3*01 | YF | | | 3568 |
| LE1 | Neo⁺WT⁺ | DHX33- | DHX33- | 0 | 0 | 0 | | | | | | | | | |
| | | LLA_M4I | LLA | | | | | | | | | | | | |
| LE10 | Neo⁺WT⁺ | FNDC3B- | FNDC3B- | 0 | 0 | 0 | 8-6*01 | CAVTDNNAG | | | 7-3*01 | CASSFGPGY | | | 3087 |
| | | VVL_L3M | VVL | | | | | NMLTF | | | | EQYF | | | 3569 |
| LE3 | Neo⁺WT⁺ | OR5M3- | OR5M3- | 0 | 0 | 0 | | | | | | | | | |
| | | KMV_T8N | KMV | | | | | | | | | | | | |
| LE7 | Neo⁺WT⁺ | C15orf32- | PHKA2- | 0 | 0 | 0 | | | | | 6-6*01 | CASSYARDR | | | 3570 |
| | | MLS_G9R | LLS_SF | | | | | | | | | NTEAFF | | | |
| LE9 | Neo⁺WT⁺ | BTBD1- | BTBD1- | 0 | 0 | 0 | 6*01 | CALDILISG | | | 30*01 | CAGWDRTP | | | 3088 |
| | | FML_LI | FML | | | | | GSYIPTF | | | | YEQYF | | | 3571 |
| LF11 | Neo⁺WT⁺ | BTBD1- | BTBD1- | 0 | 0 | 0 | 19*01 | CALSSPTYN | | | 2*01 | CASSEDAGN | | | 3089 |
| | | FML_LI | FML | | | | | NNDMRF | | | | YGYTF | | | 3572 |
| LF12 | Neo⁺WT⁺ | 0 | 0 | 0 | 0 | 0 | 24*01 | CAFESGGGA | | | | | | | 3090 |
| | | | | | | | | DGLTF | | | | | | | |
| LG1 | Neo⁺WT⁺ | OR5M3- | 0 | 0 | 0 | 0 | | | | | 6-1*01 | CASSEIQAFE | | | 3573 |
| | | KMV_T8N | | | | | | | | | | ETQYF | | | |
| LG12 | Neo⁺WT⁺ | PXDNL- | PXDNL- | 0 | 0 | 0 | 12-2*01 | CAVRGGND | 3*01F | CAGFGNV | 28*01 | CASSLFARG | | | 3091 |
| | | SIL_S1F | SIL | | | | | MRF | | LHC | | GPTDTQYF | | | 3456 |
| | | | | | | | | | | | | | | | 3574 |
| LG2 | Neo⁺WT⁺ | TRPV4- | ST6GALNAC2- | TRPV4- | 0 | 0 | 12-2*01 | CAVNTRTAL | | | 19*01 | CASSFGSGN | | | 3092 |
| | | FMI | LLF_Y6H | FMI_A6T | | | | IF | | | | TIYF | | | 3575 |
| LG8 | Neo⁺WT⁺ | GALC- | GALC-YVV | 0 | 0 | 0 | 38-2/ | CACMDSNY | | | 7-9*01 | CASSPHSGG | | | 3093 |
| | | YVV_V3L | | | | | DV8*01 | QLIW | | | | DPRNEQFF | | | 3576 |
| LH10 | Neo⁺WT⁺ | 0 | 0 | 0 | 0 | 0 | 8-4*01 | CAVTLTGGG | | | | | | | 3094 |
| | | | | | | | | NKLTF | | | | | | | |
| LH2 | Neo⁺WT⁺ | APBB2- | RYR3- | ZDHHC17- | 0 | 0 | | | | | | | | | |
| | | VQY_L7F | VLN_E6K | LLL_T4I | | | | | | | | | | | |
| LH4 | Neo⁺WT⁺ | NSDHL- | NSDHL- | 0 | 0 | 0 | 14/ | CAMRELSGN | 41*01 | CAVEGSRL | 9*01 | CASSVGGGH | | | 3095 |
| | | ILT_A9V | ILT | | | | DV4*01 | YGGSQGNLI | | TF | | QPQHF | | | 3025 |
| | | | | | | | | F | | | | | | | 3577 |
| LH6 | Neo⁺WT⁺ | CLCN4- | CLCN4- | 0 | 0 | 0 | 12-3*01 | CAMSVPGYS | | | 2*01 | CANGQGDY | | | 3096 |
| | | LLA_G8V | LLA | | | | | TLTF | | | | EQYF | | | 3578 |
| LH8 | Neo⁺WT⁺ | NSDHL- | 0 | 0 | 0 | 0 | | | | | | | | | |
| | | ILT_A9V | | | | | | | | | | | | | |
| LH9 | Neo⁺WT⁺ | PLXNB1- | PLXNB1- | 0 | 0 | 0 | | | | | | | | | |
| | | VLF_V1L | VLF | | | | | | | | | | | | |
| MA2 | Neo⁺WT⁺ | CNKSR1- | CNKSR1- | 0 | 0 | 0 | 14/ | CAMREGNT | | | 19*01 | CASSETSGLI | | | 3097 |
| | | SLA_A9V | SLA | | | | DV4*01 | GGFKTIF | | | | DEKLFF | | | 3579 |
| MA3 | Neo⁺WT⁺ | KCNC3- | KCNC3- | EXOC3L4- | 0 | 0 | | | | | 7-9*01 | CASSLAYRP | | | 3580 |
| | | FLP_A7V | FLP | ILL_V9I | | | | | | | | YEQYF | | | |
| MA5 | Neo⁺WT⁺ | SLC1A2- | SLC1A2- | DRAM1- | 0 | 0 | | | | | 2*01 | CASSWTGDS | | | 3581 |
| | | YMS_S3P | YMS | FII_I3F | | | | | | | | NQPQHF | | | |
| MA7 | Neo⁺WT⁺ | OVOL1- | OVOL1- | 0 | 0 | 0 | 12-2*01 | CAVNAPGTY | | | 12-3*01, | CASSPPDQV | | | 3098 |
| | | SLL_L9V | SLL | | | | | KYIF | | | 12-4*01 | YNEQFF | | | 3582 |
| MA8 | Neo⁺WT⁺ | STOX1- | STOX1- | 0 | 0 | 0 | 41*01 | CAVSYDSNY | | | 7-9*01 | CASSSNIWS | | | 3099 |
| | | RLM_M3I | RLM | | | | | QLIW | | | | PDTQYF | | | 3583 |
| MB4 | Neo⁺WT⁺ | 0 | 0 | 0 | 0 | 0 | 8-3*01 | CAVGARNTG | | | 12-3*01, | CASSPWDSS | | | 3100 |
| | | | | | | | | FQKLVF | | | 12-4*01 | GELFF | | | 3584 |
| MD3 | Neo⁺WT⁺ | HCV- | HCV- | 0 | 0 | 0 | | | | | 3-1*01 | CASSYYSGQ | | | 3585 |
| | | KLV(APC) | KLV(PE) | | | | | | | | | GNEKLFF | | | |
| MD5 | Neo⁺WT⁺ | 0 | 0 | 0 | 0 | 0 | 12-2*01 | CAAATGGGN | 9-2*01 | CALTASNQ | 27*01 | CASSLGGHQ | | | 3101 |
| | | | | | | | | KLTF | | AGTALIF | | PQHF | | | 3457 |
| | | | | | | | | | | | | | | | 3586 |
| ME3 | Neo⁺WT⁺ | BAIAP3- | BAIAP3- | 0 | 0 | 0 | | | | | 13*01 | CASTESSYN | | | 3587 |
| | | ILN_V6I | ILN | | | | | | | | | EQFF | | | |
| ME8 | Neo⁺WT⁺ | ITIH6- | ITIH6- | 0 | 0 | 0 | 12-2*01 | CAVKGGSQ | | | 9*01 | CASSVQSTD | | | 3102 |
| | | RLG_G3V | RLG | | | | | GNLIF | | | | TQYF | | | 3588 |
| MF11 | Neo⁺WT⁺ | 0 | 0 | 0 | 0 | 0 | 41*01 | CAVRPTSPY | | | | | | | 3103 |
| | | | | | | | | GGSQGNLIF | | | | | | | |
| MF4 | Neo⁺WT⁺ | 0 | 0 | 0 | 0 | 0 | | | | | 13*01 | CASSSTVGV | | | 3589 |
| | | | | | | | | | | | | RDYHSGNTI | | | |
| | | | | | | | | | | | | YF | | | |
| MF7 | Neo⁺WT⁺ | 0 | 0 | 0 | 0 | 0 | 12-2*01 | CAVKGTDKLI | | | 2*01 | CASTDLSDT | | | 3104 |
| | | | | | | | | F | | | | QYF | | | 3590 |
| MG10 | Neo⁺WT⁺ | 0 | 0 | 0 | 0 | 0 | | | | | | | | | |
| MG12 | Neo⁺WT⁺ | GALC- | GALC- | 0 | 0 | 0 | 6*01 | CALGTHDMR | | | 10-1*01 | CASSESGAA | | | 3105 |
| | | YVV_V3L | YVV | | | | | F | | | | YTGELFF | | | 3591 |
| MG3 | Neo⁺WT⁺ | 0 | 0 | 0 | 0 | 0 | | | | | 3-1*01 | CATERGFRT | | | 3592 |
| | | | | | | | | | | | | DTQYF | | | |
| MG6 | Neo⁺WT⁺ | OVOL1- | OVOL1- | 0 | 0 | 0 | 41*01 | CAVEGSRLT | | | | | | | 3025 |
| | | SLL_L9V | SLL | | | | | F | | | | | | | |
| MH10 | Neo⁺WT⁺ | 0 | 0 | 0 | 0 | 0 | | | | | 6-5*01 | CASSYEQGP | | | 3593 |
| | | | | | | | | | | | | YEQYF | | | |
| MH12 | Neo⁺WT⁺ | 0 | 0 | 0 | 0 | 0 | | | | | 3-1*01 | CASSQAYGG | | | 3594 |
| | | | | | | | | | | | | DSSYEQYF | | | |
| MH9 | Neo⁺WT⁺ | PHKA2- | KCNB2- | 0 | 0 | 0 | | | | | | | | | |
| | | LLS_SF | LLA_P6T | | | | | | | | | | | | |
| NA11 | Neo⁺WT⁺ | 0 | 0 | 0 | 0 | 0 | 1-2*01 | CARMSTDS | | | 2*01 | CASGRSGGV | | | 3106 |
| | | | | | | | | WGKLQF | | | | GRNGYTF | | | 3595 |
| NA2 | Neo⁺WT⁺ | DHX33- | 0 | 0 | 0 | 0 | | | | | 9*01 | CASALGSGG | | | 3596 |
| | | LLA_K5T | | | | | | | | | | AYEQFF | | | |
| NA3 | Neo⁺WT⁺ | CELSR1- | NOS1- | MRGPRF- | 0 | 0 | 8-6*01 | CAAFMFSGG | | | 27*01 | CASTLGQGN | | | 3107 |
| | | YLF_F3L | FID_D3Y | RLW_R1W | | | | YNKLIF | | | | TEAFF | | | 3597 |
| NA6 | Neo⁺WT⁺ | 0 | 0 | 0 | 0 | 0 | | | | | 3-1*01 | CASSQDTGS | | | 3598 |
| | | | | | | | | | | | | GNTIYF | | | |
| NA9 | Neo⁺WT⁺ | PHKA2- | 0 | 0 | 0 | 0 | 12-1*01 | CVVSNQAGT | | | 4-2*01 | CASSQGPGT | | | 3108 |
| | | LLS_SF | | | | | | ALIF | | | | GFEGYTF | | | 3599 |
| NB11 | Neo⁺WT⁺ | 0 | 0 | 0 | 0 | 0 | 21*01 | CAVRFNTGF | | | 27*01 | CASRRGPTD | | | 3109 |
| | | | | | | | | QKLVF | | | | TQYF | | | 3600 |
| NB12 | Neo⁺WT⁺ | KIF20B- | OR8B8- | A2ML1- | A2ML1- | 0 | 25*01 | CAGRGMVG | | | 2*01 | CASSALAGG | | | 3110 |
| | | YTS_S6L | YVN | YLD_K7R | YLD_WT | | | NKLVF | | | | YNEQFF | | | 3601 |
| NB3 | Neo⁺WT⁺ | CNKSR1- | CNKSR1- | 0 | 0 | 0 | 4*01 | CLIRDDKII | | | 20-1*01 | CSAPKEEPY | | | 3111 |
| | | SLA_A9V | SLA | | | | | F | | | | GYTF | | | 3602 |
| NB4 | Neo⁺WT⁺ | HTR1F- | 0 | 0 | 0 | 0 | 10*01 | CVVMPPGS | 1-2*01 | CAVTVVDN | 27*01 | CASSLTGSA | | | 3112 |
| | | 9_V1M | | | | | | GYSTLTF | | NARLMF | | EAFF | | | 3458 |
| | | | | | | | | | | | | | | | 3603 |
| NB5 | Neo⁺WT⁺ | ATP6AP1- | HCV- | HCV- | 0 | 0 | | | | | | | | | |
| | | KLG_G3W | KLV(APC) | KLV(PE) | | | | | | | | | | | |
| NB6 | Neo⁺WT⁺ | HTR1F- | OR5M3- | 0 | 0 | 0 | 14/ | CAMRETDSS | 8-6*01 | CAVTPNFN | 29-1*01 | CSVERGGDE | | | 3113 |
| | | 10_V1M | KMV | | | | DV4*01 | YKLIF | | KFYF | | QFF | | | 3459 |
| | | | | | | | | | | | | | | | 3604 |
| NB8 | Neo⁺WT⁺ | 0 | 0 | 0 | 0 | 0 | 17*01 | CATGGPDM | | | 6-2*01, | CASSYSISG | | | 3114 |
| | | | | | | | | RF | | | 6-3*01 | QGGETQYF | | | 3605 |
| NC10 | Neo⁺WT⁺ | DHX33- | DHX33- | DHX33- | HCV- | 0 | | | | | 7-8*01 | CASSGRQGS | | | 3606 |
| | | LLA_K5T | LLA_M4I | LLA | KLV(APC) | | | | | | | YEQYF | | | |
| NC7 | Neo⁺WT⁺ | OR2T1- | OR2T1- | 0 | 0 | 0 | 19*01 | CALKNLGNY | | | 20-1*01 | CSAPSYREL | | | 3115 |
| | | FLN_F5L | FLN | | | | | GQNFVF | | | | AGAYLQETQ | | | 3607 |
| | | | | | | | | | | | | YF | | | |
| NC8 | Neo⁺WT⁺ | BAIAP3- | BAIAP3- | 0 | 0 | 0 | 20*01 | CAVQAGNTD | 4*01 | CLVGDLTS | 20-1*01 | CSARTWTGN | | | 3116 |
| | | ILN_V6I | ILN | | | | | KLIF | | FQGAQKL | | TIYF | | | 3460 |
| | | | | | | | | | | VF | | | | | 3608 |
| ND11 | Neo⁺WT⁺ | HTR1F- | HTR1F-9 | 0 | 0 | 0 | 29/ | CAASANNQG | | | 12-3*01, | CASSLVAGP | | | 3117 |
| | | 9_V1M | | | | | DV5*01 | GKLIF | | | 12-4*01 | YSQETQYF | | | 3609 |
| ND12 | Neo⁺WT⁺ | 0 | 0 | 0 | 0 | 0 | | | | | 13*01 | CASSPRTGV | | | 3610 |
| | | | | | | | | | | | | GEQYF | | | |
| ND3 | Neo⁺WT⁺ | ITIH6- | PIGN- | 0 | 0 | 0 | | | | | | | | | |
| | | RLG_G3V | FLT_P7H | | | | | | | | | | | | |
| ND7 | Neo⁺WT⁺ | PHKA2- | PHKA2- | 0 | 0 | 0 | 12-1*01 | CVVGPGANN | 9-2*01 | CALSMYS | 12-3*01, | CASSFRQTL | | | 3118 |
| | | LLS_SF | LLS | | | | | LFF | | GGGADGL | 12-4*01 | AVYEQYF | | | 3461 |
| | | | | | | | | | | TF | | | | | 3611 |
| NE1 | Neo⁺WT⁺ | GCN1L1-9 | APBB2- | GPR174- | 0 | 0 | | | | | | | | | |
| | | | VQY | FSF | | | | | | | | | | | |
| NE11 | Neo⁺WT⁺ | C17orf75- | 0 | 0 | 0 | 0 | 12-2*01 | CAVSTGGGA | | | 5-4*01 | CASSLGQEIP | | | 3119 |
| | | ALS_V7A | | | | | | DGLTF | | | | YYGYTF | | | 3612 |
| NE4 | Neo⁺WT⁺ | CHD8- | 0 | 0 | 0 | 0 | | | | | | | | | |
| | | KLN_P7A | | | | | | | | | | | | | |
| NE8 | Neo⁺WT⁺ | BAIAP3- | BAIAP3- | 0 | 0 | 0 | 26-1*01 | CIVRVAGQF | | | 11-2*01 | CASSSQGGA | | | 3120 |
| | | ILN_V6I | ILN | | | | | YF | | | | KNEQYF | | | 3613 |
| NF12 | Neo⁺WT⁺ | PRSS16- | OR10A3- | 0 | 0 | 0 | 5*01 | CAETPNDYK | 1-2*01 | CAVRDYY | 28*01 | CASSLVGAD | | | 3121 |
| | | LLL_L1Q | ILI | | | | | LSF | | QLIW | | RSGELFF | | | 3462 |
| | | | | | | | | | | | | | | | 3614 |
| NF4 | Neo⁺WT⁺ | 0 | 0 | 0 | 0 | 0 | | | | | | | | | |
| NG2 | Neo⁺WT⁺ | IL17RA- | 0 | 0 | 0 | 0 | | | | | | | | | |
| | | FIT_TM | | | | | | | | | | | | | |
| NG3 | Neo⁺WT⁺ | 0 | 0 | 0 | 0 | 0 | | | | | | | | | |
| NG5 | Neo⁺WT⁺ | DHX33- | HTR1F- | 0 | 0 | 0 | | | | | | | | | |
| | | LLA_K5T | LVM_V2M | | | | | | | | | | | | |
| NG9 | Neo⁺WT⁺ | HBZ- | GABRG3- | GABRG3- | OR8B8- | 0 | 38-1*01 | CAFDFSSGS | | | 19*01 | CASSYGQPN | | | 3122 |
| | | KLS_A7T | YVT | YVT_L7I | YVN_V2L | | | ARQLTF | | | | TEAFF | | | 3615 |
| NH11 | Neo⁺WT⁺ | GABRG3- | GABRG3- | 0 | 0 | 0 | 12-2*01 | CAVNRLVF | | | | | | | 3123 |
| | | YVT_L7I | YVT | | | | | | | | | | | | |
| NH2 | Neo⁺WT⁺ | 0 | 0 | 0 | 0 | 0 | 12-2*01 | CAVTKNTGN | | | 20-1*01 | CSARTGNTN | | | 3124 |
| | | | | | | | | QFYF | | | | EQFF | | | 3616 |
| NH3 | Neo⁺WT⁺ | CD47- | CD47- | 0 | 0 | 0 | | | | | | | | | |
| | | GLT_V6F | GLT | | | | | | | | | | | | |
| NH5 | Neo⁺WT⁺ | 0 | 0 | 0 | 0 | 0 | | | | | | | | | |
| OA1 | Neo⁺WT⁺ | CNKSR1- | CNKSR1- | 0 | 0 | 0 | 14/ | CAMSVSSND | | | 3-1*01 | CASSQGTGG | | | 3125 |
| | | SLA | SLA_A9V | | | | DV4*01 | YKLSF | | | | IVDIQYF | | | 3617 |
| OA5 | Neo⁺WT⁺ | 0 | 0 | 0 | 0 | 0 | 21*01 | CAVRLGGSY | | | 11-2*01 | CASRDILYNE | | | 3126 |
| | | | | | | | | IPTF | | | | QFF | | | 3618 |
| OB11 | Neo⁺WT⁺ | 0 | 0 | 0 | 0 | 0 | 12-3*01 | CAMSGDYKL | | | 28*01 | CASSSQSSG | | | 3127 |
| | | | | | | | | SF | | | | ANVLTF | | | 3619 |
| OB4 | Neo⁺WT⁺ | 0 | 0 | 0 | 0 | 0 | | | | | | | | | |
| OB7 | Neo⁺WT⁺ | TRPC1- | TRPC1- | VN1R5- | 0 | 0 | 3*01F | CGSADRGST | | | | | | | 3128 |
| | | MLL_Q5H | MLL | MII_S7Y | | | | LGRLYF | | | | | | | |
| OC10 | Neo⁺WT⁺ | 0 | 0 | 0 | 0 | 0 | | | | | 4-2*01 | CASSQMTG | | | 3620 |
| | | | | | | | | | | | | GGEQFF | | | |
| OC3 | Neo⁺WT⁺ | 0 | 0 | 0 | 0 | 0 | | | | | 11-2*01 | CASSPGGEA | | | 3621 |
| | | | | | | | | | | | | FF | | | |
| OC4 | Neo⁺WT⁺ | ITIH6- | ITIH6- | 0 | 0 | 0 | 19*01 | CALSEAEGY | | | | | | | 3129 |
| | | RLG_G3V | RLG | | | | | SGYALNF | | | | | | | |
| OD11 | Neo⁺WT⁺ | 0 | 0 | 0 | 0 | 0 | | | | | 7-9*01 | CASSLVRQE | | | 3622 |
| | | | | | | | | | | | | AAGELFF | | | |
| OD3 | Neo⁺WT⁺ | OR8B8- | 0 | 0 | 0 | 0 | | | | | | | | | |
| | | YVN_V2L | | | | | | | | | | | | | |
| OD4 | Neo⁺WT⁺ | GABRG3- | 0 | 0 | 0 | 0 | 27*01 | CAGVFGGSN | | | 9*01 | CASSGGQG | | | 3130 |
| | | TAM_L5I | | | | | | YKLTF | | | | WTDTQYF | | | 3623 |
| OD6 | Neo⁺WT⁺ | 0 | 0 | 0 | 0 | 0 | | | | | | | | | |
| OD7 | Neo⁺WT⁺ | VN1R5- | VN1R5- | 0 | 0 | 0 | 24*01 | CAFILVANAG | | | 12-3*01, | CASRPRQVE | | | 3131 |
| | | MII_S7Y | MII | | | | | KSTF | | | 12-4*01 | TQYF | | | 3624 |
| OD8 | Neo⁺WT⁺ | NSDHL- | NSDHL- | 0 | 0 | 0 | 14/ | CAMREVAGA | | | 10-2*01 | CASGTLNSN | | | 3132 |
| | | ILT | ILT_A9V | | | | DV4*01 | GNKLTF | | | | QPQHF | | | 3625 |
| OE1 | Neo⁺WT⁺ | TEAD1- | NSDHL- | NSDHL- | 0 | 0 | | | | | | | | | |
| | | VLE | ILT | ILT_A9V | | | | | | | | | | | |
| OE10 | Neo⁺WT⁺ | HCV- | HCV- | 0 | 0 | 0 | 38-2/ | CAYGEDDKII | | | 25-1*01 | CASRRDSSG | | | 3133 |
| | | KLV(APC) | KLV(PE) | | | | DV8*01 | F | | | | YTF | | | 3626 |
| OE2 | Neo⁺WT⁺ | 0 | 0 | 0 | 0 | 0 | | | | | | | | | |
| OE3 | Neo⁺WT⁺ | 0 | 0 | 0 | 0 | 0 | | | | | | | | | |
| OE8 | Neo⁺WT⁺ | DHX33- | 0 | 0 | 0 | 0 | 16*01 | CALRFNSSY | | | | | | | 3134 |
| | | LLA_K5T | | | | | | KLIF | | | | | | | |
| OF10 | Neo⁺WT⁺ | HTR1F- | 0 | 0 | 0 | 0 | | | | | 29-1*01 | CSVEQGGDT | | | 3627 |
| | | 10_V1M | | | | | | | | | | QYF | | | |
| OF6 | Neo⁺WT⁺ | 0 | 0 | 0 | 0 | 0 | | | | | | | | | |
| OF7 | Neo⁺WT⁺ | PIGN- | 0 | 0 | 0 | 0 | | | | | | | | | |
| | | FLT_P7H | | | | | | | | | | | | | |
| OF8 | Neo⁺WT⁺ | HTR1F- | 0 | 0 | 0 | 0 | 5*01 | CAESKESGG | | | 27*01 | CASSGFSNQ | | | 3135 |
| | | 9_V1M | | | | | | YQKVTF | | | | PQHF | | | 3628 |
| OF9 | Neo⁺WT⁺ | 0 | 0 | 0 | 0 | 0 | | | | | 2*01 | CATLWGTDT | | | 3629 |
| | | | | | | | | | | | | QYF | | | |
| OG5 | Neo⁺WT⁺ | 0 | 0 | 0 | 0 | 0 | | | | | 6-2*01, | CASSYIPGR | | | 3630 |
| | | | | | | | | | | | 6-3*01 | YEQYF | | | |
| OG7 | Neo⁺WT⁺ | AGXT2L2- | 0 | 0 | 0 | 0 | 14/ | CAMREPRG | | | | | | | 3136 |
| | | ILT_M5I | | | | | DV4*01 | GRRALTF | | | | | | | |
| OH10 | Neo⁺WT⁺ | 0 | 0 | 0 | 0 | 0 | 19*01 | CALRGFQDS | | | | | | | 3137 |
| | | | | | | | | NYQLIW | | | | | | | |
| OH11 | Neo⁺WT⁺ | PHKA2- | 0 | 0 | 0 | 0 | 12-2*01 | CAVTSDGQK | | | 5-4*01 | CASSLEGEK | | | 3138 |
| | | LLS_SF | | | | | | LLF | | | | LFF | | | 3631 |
| OH2 | Neo⁺WT⁺ | OR6F1- | 0 | 0 | 0 | 0 | | | | | | | | | |
| | | VLN_T8M | | | | | | | | | | | | | |
| OH4 | Neo⁺WT⁺ | 0 | 0 | 0 | 0 | 0 | | | | | | | | | |
| OH8 | Neo⁺WT⁺ | GALC- | 0 | 0 | 0 | 0 | | | | | | | | | |
| | | YVV_V3L | | | | | | | | | | | | | |
| OH9 | Neo⁺WT⁺ | BAIAP3- | BAIAP3- | 0 | 0 | 0 | | | | | | | | | |
| | | ILN_V6I | ILN | | | | | | | | | | | | |
| SA10 | Neo⁺WT⁺ | HTR1F- | HTR1F-9 | 0 | 0 | 0 | 26-2*01 | CILRDPYNTD | | | | | | | 3139 |
| | | 9_V1M | | | | | | KLIF | | | | | | | |
| SA11 | Neo⁺WT⁺ | 0 | 0 | 0 | 0 | 0 | | | | | | | | | |
| SA4 | Neo⁺WT⁺ | OR5M3- | OR5M3- | 0 | 0 | 0 | 38-2/ | CAYRTGDSG | | | 10-1*01 | CASSEFRDR | | | 3140 |
| | | KMV | KMV_T8N | | | | DV8*01 | AGSYQLTF | | | | NQPQHF | | | 3632 |
| SA6 | Neo⁺WT⁺ | 0 | 0 | 0 | 0 | 0 | | | | | 4-2*01 | CASSQGRR | | | 3633 |
| | | | | | | | | | | | | GGGDKNIQY | | | |
| | | | | | | | | | | | | F | | | |
| SC10 | Neo⁺WT⁺ | 0 | 0 | 0 | 0 | 0 | | | | | | | | | |
| SC11 | Neo⁺WT⁺ | 0 | 0 | 0 | 0 | 0 | | | | | | | | | |
| SC6 | Neo⁺WT⁺ | 0 | 0 | 0 | 0 | 0 | | | | | | | | | |
| SC9 | Neo⁺WT⁺ | 0 | 0 | 0 | 0 | 0 | | | | | | | | | |
| SD10 | Neo⁺WT⁺ | 0 | 0 | 0 | 0 | 0 | | | | | | | | | |
| SD11 | Neo⁺WT⁺ | ITIH6- | ITIH6- | 0 | 0 | 0 | | | | | 20-1*01 | CSARSEKSG | | | 3634 |
| | | RLG_G3V | RLG | | | | | | | | | ANVLTF | | | |
| SD4 | Neo⁺WT⁺ | CNKSR1- | CNKSR1- | 0 | 0 | 0 | | | | | | | | | |
| | | SLA_A9V | SLA | | | | | | | | | | | | |
| SD6 | Neo⁺WT⁺ | HCV- | HCV- | 0 | 0 | 0 | 38-1*01 | CAFIWNDYK | | | 19*01 | CASSSGGG | | | 3141 |
| | | KLV(PE) | KLV(APC) | | | | | LSF | | | | QPQHF | | | 3635 |
| SE10 | Neo⁺WT⁺ | STOX1- | STOX1- | 0 | 0 | 0 | 17*01 | CATDAEDSN | | | 27*01 | CASSSSSGD | | | 3142 |
| | | RLM_M3I | RLM | | | | | YQLIW | | | | EQYF | | | 3636 |
| SE12 | Neo⁺WT⁺ | PGM5- | 0 | 0 | 0 | 0 | | | | | | | | | |
| | | AVG_H5Y | | | | | | | | | | | | | |
| SE7 | Neo⁺WT⁺ | TBX3- | TBX3- | 0 | 0 | 0 | 14/ | CAMREAFAG | | | 10-3*01 | CAISELDWG | | | 3143 |
| | | GMG | GMG_T8M | | | | DV4*01 | TASKLTF | | | | VSSPLHF | | | 3637 |
| SF11 | Neo⁺WT⁺ | HTR1F- | HTR1F-9 | 0 | 0 | 0 | 5*01 | CAEIGVGGY | | | 6-5*01 | CATSPSLGT | | | 3144 |
| | | 9_V1M | | | | | | QKVTF | | | | QYF | | | 3638 |
| SF12 | Neo⁺WT⁺ | GABRG3- | GABRG3-YVT | 0 | 0 | 0 | | | | | | | | | |
| | | YVT_L7I | | | | | | | | | | | | | |
| SF5 | Neo⁺WT⁺ | BTBD1- | BTBD1- | 0 | 0 | 0 | | | | | | | | | |
| | | FML_LI | FML | | | | | | | | | | | | |
| SF7 | Neo⁺WT⁺ | 0 | 0 | 0 | 0 | 0 | | | | | | | | | |
| SG7 | Neo⁺WT⁺ | 0 | 0 | 0 | 0 | 0 | | | | | | | | | |
| SH4 | Neo⁺WT⁺ | OR6F1- | OR6F1- | 0 | 0 | 0 | | | | | 15*01 | CATSKTADR | | | 3639 |
| | | VLN_T8M | VLN | | | | | | | | | SPYEQYF | | | |
| SH6 | Neo⁺WT⁺ | OR6F1- | OR6F1- | 0 | 0 | 0 | 29/ | CAASGAGGT | | | 3-1*01 | CASSQEGRQ | | | 3145 |
| | | VLN_T8M | VLN | | | | DV5*01 | SYGKLTF | | | | GSYNEQFF | | | 3640 |
| SH9 | Neo⁺WT⁺ | 0 | 0 | 0 | 0 | 0 | | | | | | | | | |
| GA1 | Neo⁺WT⁻ | HTR1F- | 0 | 0 | 0 | 0 | 19*01 | CALSEASRD | | | 19*01 | CASRPGQVV | | | 3146 |
| | | 10_V1M | | | | | | FQKLVF | | | | YGYTF | | | 3641 |
| GA5 | Neo⁺WT⁻ | ITIH6- | 0 | 0 | 0 | 0 | | | | | 5-1*01 | CASSLKTDS | | | 3642 |
| | | RLG_G3V | | | | | | | | | | TPLQETQYF | | | |
| GA7 | Neo⁺WT⁻ | 0 | 0 | 0 | 0 | 0 | | | | | | | | | |
| GA9 | Neo⁺WT⁻ | SEC24A- | 0 | 0 | 0 | 0 | 38-2/ | CAYTSNDMR | | | 4-2*01 | CASSQGTSG | | | 3147 |
| | | FLY_P5L | | | | | DV8*01 | F | | | | TDTQYF | | | 3646 |
| GB11 | Neo⁺WT⁻ | PIGN- | 0 | 0 | 0 | 0 | 12-2*01 | CAVPLAGGT | | | 30*01 | CAWSWTVN | | | 3148 |
| | | FLT_P7H | | | | | | SYGKLTF | | | | TEAFF | | | 3644 |
| GB12 | Neo⁺WT⁻ | ERBB2- | 0 | 0 | 0 | 0 | 19*01 | CALSEAGYS | | | 29-1*01 | CSVVGTGSV | | | 3149 |
| | | ALI_H8Y | | | | | | SASKIIF | | | | ITNEKLFF | | | 3645 |
| GB9 | Neo⁺WT⁻ | ATP6AP1- | 0 | 0 | 0 | 0 | 35*01 | CAGLPDQTG | | | | | | | 3150 |
| | | KLG_G3W | | | | | | ANNLFF | | | | | | | |
| GC2 | Neo⁺WT⁻ | PHKA2- | 0 | 0 | 0 | 0 | | | | | | | | | |
| | | LLS_SF | | | | | | | | | | | | | |
| GC4 | Neo⁺WT⁻ | SEC24A- | 0 | 0 | 0 | 0 | 22*01 | CAVAYSGGG | | | 7-9*01 | CASSSDLRT | | | 3151 |
| | | FLY_P5L | | | | | | ADGLTF | | | | NYNEQFF | | | 3646 |
| GC6 | Neo⁺WT⁻ | SEC24A- | 0 | 0 | 0 | 0 | 12-1*01 | CVVNGNND | 41*01 | CAVEGSRL | 4-1*01 | CASSQDEGY | | | 3152 |
| | | FLY_P5L | | | | | | MRF | | TF | | EQYF | | | 3025 |
| | | | | | | | | | | | | | | | 3647 |
| GC9 | Neo⁺WT⁻ | OR6F1- | 0 | 0 | 0 | 0 | 12-2*01 | CAASSSNTG | | | 4-2*01 | CASSQDLNE | | | 3153 |
| | | VLN_T8M | | | | | | KLIF | | | | QYF | | | 3648 |
| GD11 | Neo⁺WT⁻ | OR10A3- | 0 | 0 | 0 | 0 | 8-6*01 | CAVSDLAGQ | | | 19*01 | CASSPVGDT | | | 3154 |
| | | ILI_V6F | | | | | | KLLF | | | | QYF | | | 3649 |
| GD3 | Neo⁺WT⁻ | PLXNB1- | 0 | 0 | 0 | 0 | 12-3*01 | CAMGDYKLS | | | | | | | 3155 |
| | | VLF_V1L | | | | | | F | | | | | | | |
| GD4 | Neo⁺WT⁻ | CLCN4- | 0 | 0 | 0 | 0 | 12-3*01 | CAMSAGNQ | | | 11-2*01 | CASSLDLAG | | | 3156 |
| | | LLA_G8V | | | | | | GGKLIF | | | | GFYEQYF | | | 3650 |
| GE4 | Neo⁺WT⁻ | 0 | 0 | 0 | 0 | 0 | 13-1*01 | CAASSPLNA | | | | | | | 3157 |
| | | | | | | | | GGTSYGKLT | | | | | | | |
| | | | | | | | | F | | | | | | | |
| GE5 | Neo⁺WT⁻ | CHST14- | 0 | 0 | 0 | 0 | | | | | | | | | |
| | | MLM_F4L | | | | | | | | | | | | | |
| GE6 | Neo⁺WT⁻ | DHX33- | 0 | 0 | 0 | 0 | | | | | 20-1*01 | CSARDPQGF | | | 3651 |
| | | LLA_M4I | | | | | | | | | | DGYTF | | | |
| GF11 | Neo⁺WT⁻ | PHKA2- | 0 | 0 | 0 | 0 | 41*01 | CAVEGSRLT | 12-2*01 | CAVRGGK | | | | | 3025 |
| | | LLS_SF | | | | | | F | | LTF | | | | | 3463 |
| GF4 | Neo⁺WT⁻ | IGF1- | 0 | 0 | 0 | 0 | | | | | | | | | |
| | | TMS_S4F | | | | | | | | | | | | | |
| GF5 | Neo⁺WT⁻ | KCNB2- | 0 | 0 | 0 | 0 | 12-2*01 | CAATGGSYI | | | 14*01 | CASSQAGEQ | | | 3158 |
| | | LLA_P6T | | | | | | PTF | | | | YF | | | 3652 |
| GF9 | Neo⁺WT⁻ | VN1R2- | 0 | 0 | 0 | 0 | 12-2*01 | CAVFGLSND | | | | | | | 3159 |
| | | LML_L3F | | | | | | YKLSF | | | | | | | |
| GG2 | Neo⁺WT⁻ | DHX33- | 0 | 0 | 0 | 0 | | | | | | | | | |
| | | LLA_M4I | | | | | | | | | | | | | |
| GG6 | Neo⁺WT⁻ | NOS1- | 0 | 0 | 0 | 0 | | | | | | | | | |
| | | FID_D3Y | | | | | | | | | | | | | |
| GG8 | Neo⁺WT⁻ | OR5M3- | 0 | 0 | 0 | 0 | 12-2*01 | CAVNAPDGQ | | | | | | | 3160 |
| | | KMV_T8N | | | | | | KLLF | | | | | | | |
| GG9 | Neo⁺WT⁻ | ZDHHC7- | 0 | 0 | 0 | 0 | 12-2*01 | CAVPEGNTP | | | 18*01 | CASSPYGNTI | | | 3161 |
| | | SLL_P7L | | | | | | LVF | | | | YF | | | 3653 |
| GH1 | Neo⁺WT⁻ | VN1R2- | 0 | 0 | 0 | 0 | | | | | 27*01 | CASSPPGTY | | | 3654 |
| | | LML_L3F | | | | | | | | | | NEQFF | | | |
| GH10 | Neo⁺WT⁻ | USP28- | 0 | 0 | 0 | 0 | | | | | 20-1*01 | CSVPSYNEQ | | | 3655 |
| | | LII_C5F | | | | | | | | | | FF | | | |
| GH12 | Neo⁺WT⁻ | C17orf75- | 0 | 0 | 0 | 0 | 1-2*01 | CAVVIGFGN | | | 5-1*01 | CASSTQGTG | | | 3162 |
| | | ALS_V7A | | | | | | VLHC | | | | VYNEQFF | | | 3656 |
| GH3 | Neo⁺WT⁻ | USP28- | 0 | 0 | 0 | 0 | | | | | | | | | |
| | | LII_C5F | | | | | | | | | | | | | |
| GH8 | Neo⁺WT⁻ | INTS1- | 0 | 0 | 0 | 0 | 12-2*01 | CAVNGYGNK | | | 15*01 | CATSRPTDW | | | 3163 |
| | | VLL_L3F | | | | | | LVF | | | | VETQYF | | | 3657 |
| IA1 | Neo⁺WT⁻ | WDR46- | 0 | 0 | 0 | 0 | 12-2*01 | CAVNQSGYS | | | 9*01 | CASSPTGNE | | | 3164 |
| | | FLT_T3I | | | | | | TLTF | | | | QFF | | | 3658 |
| IA11 | Neo⁺WT⁻ | OR5M3- | 0 | 0 | 0 | 0 | | | | | 6-6*01 | CASSYPSTG | | | 3659 |
| | | KMV_T8N | | | | | | | | | | SSYEQYF | | | |
| IA2 | Neo⁺WT⁻ | OR14C36- | 0 | 0 | 0 | 0 | 3*01 | CAVRDIDSN | | | 29-1*01 | CSVAGGTEA | | | 3165 |
| | | FML_V6L | | | | | | YQLIW | | | | FF | | | 3660 |
| IA3 | Neo⁺WT⁻ | OR6F1- | 0 | 0 | 0 | 0 | | | | | 20-1*01 | CSARGAFHE | | | 3661 |
| | | VLN_T8M | | | | | | | | | | QYF | | | |
| IA6 | Neo⁺WT⁻ | ATP6AP1- | 0 | 0 | 0 | 0 | | | | | | | | | |
| | | KLG_G3W | | | | | | | | | | | | | |
| IA7 | Neo⁺WT⁻ | VN1R2- | 0 | 0 | 0 | 0 | | | | | 12-3*01, | CASSIQGALT | | | 3662 |
| | | LML_L3F | | | | | | | | | 12-4*01 | DTQYF | | | |
| IB12 | Neo⁺WT⁻ | ATP6AP1- | 0 | 0 | 0 | 0 | 3*01 | CAVRDIGDN | | | 4-1*01 | CASSPSQGY | | | 3166 |
| | | KLG_G3W | | | | | | NDMRF | | | | GYTF | | | 3663 |
| IB2 | Neo⁺WT⁻ | MLL2- | 0 | 0 | 0 | 0 | | | | | | | | | |
| | | ALS_L8H | | | | | | | | | | | | | |
| IB8 | Neo⁺WT⁻ | MAR11- | 0 | 0 | 0 | 0 | 29/ | CAASESNFG | | | | | | | 3167 |
| | | 9_F1L | | | | | DV5*01 | NEKLTF | | | | | | | |
| IB9 | Neo⁺WT⁻ | SLC16A7- | 0 | 0 | 0 | 0 | | | | | | | | | |
| | | AMA_P6L | | | | | | | | | | | | | |
| IC1 | Neo⁺WT⁻ | MLL2- | 0 | 0 | 0 | 0 | 40*01 | CLLGDNNDM | 41*01 | CAVGEETS | 6-2*01, | CASSYFLEQ | | | 3168 |
| | | ALS_L8H | | | | | | RF | | GSRLTF | 6-3*01 | YF | | | 3464 |
| | | | | | | | | | | | | | | | 3664 |
| IC12 | Neo⁺WT⁻ | GOLGA3- | 0 | 0 | 0 | 0 | 8-3*01 | CAVGAWDS | | | 19*01 | CASSIGGQR | | | 3169 |
| | | SLD_P4L | | | | | | GGSNYKLTF | | | | YNEQFF | | | 3665 |
| IC4 | Neo⁺WT⁻ | OR14C36- | 0 | 0 | 0 | 0 | | | | | | | | | |
| | | FML_V6L | | | | | | | | | | | | | |
| IC5 | Neo⁺WT⁻ | SEC24A- | 0 | 0 | 0 | 0 | 19*01 | CALSEAGSW | | | | | | | 3170 |
| | | FLY_P5L | | | | | | GNTPLVF | | | | | | | |
| IC7 | Neo⁺WT⁻ | ZDHHC7- | 0 | 0 | 0 | 0 | | | | | | | | | |
| | | SLL_P7L | | | | | | | | | | | | | |
| ID1 | Neo⁺WT⁻ | DHX33- | 0 | 0 | 0 | 0 | | | | | | | | | |
| | | LLA_M4I | | | | | | | | | | | | | |
| ID10 | Neo⁺WT⁻ | ATP6AP1- | 0 | 0 | 0 | 0 | | | | | | | | | |
| | | KLG_G3W | | | | | | | | | | | | | |
| ID12 | Neo⁺WT⁻ | USP28- | 0 | 0 | 0 | 0 | 12-2*01 | CAVSGGYNK | | | 10-1*01 | CASSGGGA | | | 3171 |
| | | LII_C5F | | | | | | LIF | | | | GNEQFF | | | 3666 |
| ID4 | Neo⁺WT⁻ | TEAD1- | 0 | 0 | 0 | 0 | | | | | 6-1*01 | CASSEGQGY | | | 3667 |
| | | VLE_L8F | | | | | | | | | | EQYF | | | |
| ID8 | Neo⁺WT⁻ | HAUS3- | 0 | 0 | 0 | 0 | 8-4*01 | CALAGGGAD | | | 13*01 | CASSPYGQG | | | 3172 |
| | | ILN_T7A | | | | | | GLTF | | | | GRDTEAFF | | | 3668 |
| IE5 | Neo⁺WT⁻ | CD47- | 0 | 0 | 0 | 0 | 21*01 | CAVIYNFNKF | | | 2*01 | CASKSNTEA | | | 3173 |
| | | GLT_V6F | | | | | | YF | | | | FF | | | 3669 |
| IF1 | Neo⁺WT⁻ | ATP6AP1- | 0 | 0 | 0 | 0 | | | | | | | | | |
| | | KLG_G3W | | | | | | | | | | | | | |
| IF11 | Neo⁺WT⁻ | ITIH6- | 0 | 0 | 0 | 0 | | | | | | | | | |
| | | RLG_G3V | | | | | | | | | | | | | |
| IF5 | Neo⁺WT⁻ | HAUS3- | 0 | 0 | 0 | 0 | 27*01 | CAGDQNTG | | | 5-6*01 | CASSPTGSY | | | 3174 |
| | | ILN_T7A | | | | | | NQFYF | | | | GYTF | | | 3670 |
| IG11 | Neo⁺WT⁻ | PGM5- | 0 | 0 | 0 | 0 | 19*01 | CALSPRSSN | | | | | | | 3175 |
| | | AVG_H5Y | | | | | | TGKLIF | | | | | | | |
| IG6 | Neo⁺WT⁻ | TRPV3- | 0 | 0 | 0 | 0 | 21*01 | CAVKGGGA | | | 5-4*01 | CASGTELMN | | | 3176 |
| | | LLL_A8V | | | | | | DGLTF | | | | TEAFF | | | 3671 |
| IG7 | Neo⁺WT⁻ | ITIH6- | 0 | 0 | 0 | 0 | | | | | | | | | |
| | | RLG_G3V | | | | | | | | | | | | | |
| IH10 | Neo⁺WT⁻ | 0 | 0 | 0 | 0 | 0 | | | | | 25-1*01 | CASSETGYA | | | 3672 |
| | | | | | | | | | | | | YEQYF | | | |
| IH2 | Neo⁺WT⁻ | SMARCD3- | HTR1F- | GP100- | 0 | 0 | | | | | 19*01 | CATRDSQSS | | | 3673 |
| | | KLF_H8Y | 10_V1M | ALL | | | | | | | | YEQYF | | | |
| IH4 | Neo⁺WT⁻ | ATP6AP1- | 0 | 0 | 0 | 0 | 16*01 | CALSTGNQF | | | 9*01 | CASSAGQGY | | | 3177 |
| | | KLG_G3W | | | | | | YF | | | | EQYF | | | 3674 |
| IH6 | Neo⁺WT⁻ | MPV17- | 0 | 0 | 0 | 0 | 19*01 | CALKTYSNY | | | 7-9*01 | CASSLASQV | | | 3178 |
| | | YLW_A5P | | | | | | QLIW | | | | ETQYF | | | 3675 |
| JA11 | Neo⁺WT⁻ | HAUS3- | 0 | 0 | 0 | 0 | 12-2*01 | CAGFGGYQ | | | 30*01 | CAWSHSGG | | | 3179 |
| | | ILN_T7A | | | | | | KVTF | | | | YEQYF | | | 3676 |
| JA5 | Neo⁺WT⁻ | PIGN- | 0 | 0 | 0 | 0 | 12-2*01 | CAVNSNYQL | | | 20-1*01 | CSGDAFF | | | 3180 |
| | | FLT_P7H | | | | | | IW | | | | | | | 3677 |
| JA9 | Neo⁺WT⁻ | ATP6AP1- | 0 | 0 | 0 | 0 | 12-2*01 | CAVNMYGG | | | 2*01 | CASTPGTEA | | | 3181 |
| | | KLG_G3W | | | | | | YQKVTF | | | | FF | | | 3678 |
| JB10 | Neo⁺WT⁻ | INTS1- | 0 | 0 | 0 | 0 | 8-4*01 | CAVSEWDD | | | 10-2*01 | CASSDGRAD | | | 3182 |
| | | VLL_L3F | | | | | | MRF | | | | TQYF | | | 3679 |
| JB2 | Neo⁺WT⁻ | OR14C36- | 0 | 0 | 0 | 0 | | | | | | | | | |
| | | FML_V6L | | | | | | | | | | | | | |
| JB3 | Neo⁺WT⁻ | OR14C36- | 0 | 0 | 0 | 0 | 39*01 | CAVDSGGG | 27*01 | CAGADTN | 5-4*01 | CASSWLNTE | | | 3183 |
| | | FML_V6L | | | | | | ADGLTF | | AGKSTF | | AFF | | | 3465 |
| | | | | | | | | | | | | | | | 3680 |
| JB5 | Neo⁺WT⁻ | ATP6AP1- | 0 | 0 | 0 | 0 | 19*01 | CALSEAEGN | | | 9*01 | CASSVGGGS | | | 3184 |
| | | KLG_G3W | | | | | | TPLVF | | | | NQPQHF | | | 3681 |
| JC11 | Neo⁺WT⁻ | GANAB- | 0 | 0 | 0 | 0 | | | | | 2*01 | CASSGVAEW | | | 3682 |
| | | ALY_S5F | | | | | | | | | | ALETQYF | | | |
| JC8 | Neo⁺WT⁻ | TRPC1- | 0 | 0 | 0 | 0 | 2*01 | CAVEDRGG | | | 12-3*01, | CASRNTGTT | | | 3185 |
| | | MLL_Q5H | | | | | | NTGFQKLVF | | | 12-4*01 | NEKLFF | | | 3683 |
| JD10 | Neo⁺WT⁻ | DCHS1- | 0 | 0 | 0 | 0 | | | | | | | | | |
| | | TLF_I5M | | | | | | | | | | | | | |
| JD12 | Neo⁺WT⁻ | 0 | 0 | 0 | 0 | 0 | 17*01 | CATDLWSGA | | | 13*01 | CASSPTLAD | | | 3186 |
| | | | | | | | | GNMLTF | | | | EQYF | | | 3684 |
| JD8 | Neo⁺WT⁻ | ATP6AP1- | 0 | 0 | 0 | 0 | | | | | | | | | |
| | | KLG_G3W | | | | | | | | | | | | | |
| JE11 | Neo⁺WT⁻ | MPV17- | 0 | 0 | 0 | 0 | | | | | | | | | |
| | | YLW_A5P | | | | | | | | | | | | | |
| JF3 | Neo⁺WT⁻ | APBB2- | 0 | 0 | 0 | 0 | | | | | | | | | |
| | | VQY_L7F | | | | | | | | | | | | | |
| JF5 | Neo⁺WT⁻ | CELSR1- | SHROOM2- | 0 | 0 | 0 | 24*01 | CAPVSGGGA | 14/ | CAMREPY | 5-6*01 | CASSLPDRG | | | 3187 |
| | | YLF_F3L | KLL_D6V | | | | | DGLTF | DV4*01 | NAGNMLT | | GTKNIQYF | | | 3466 |
| | | | | | | | | | | F | | | | | 3685 |
| JF9 | Neo⁺WT⁻ | RYR3- | 0 | 0 | 0 | 0 | 14/ | CAMRALYYG | | | 3-1*01 | CASSLLGQS | | | 3188 |
| | | VLN_E6K | | | | | DV4*01 | KLTF | | | | TNEKLFF | | | 3686 |
| JG11 | Neo⁺WT⁻ | TRPV4- | 0 | 0 | 0 | 0 | 12-2*01 | CAVNGGWG | | | 29-1*01 | CSVDLGTEE | | | 3189 |
| | | FMI_A6T | | | | | | KLQF | | | | TQYF | | | 3687 |
| JG5 | Neo⁺WT⁻ | MPV17- | 0 | 0 | 0 | 0 | | | | | | | | | |
| | | YLW_A5P | | | | | | | | | | | | | |
| JG6 | Neo⁺WT⁻ | ATP6AP1- | 0 | 0 | 0 | 0 | | | | | | | | | |
| | | KLG_G3W | | | | | | | | | | | | | |
| JG7 | Neo⁺WT⁻ | OR14C36- | 0 | 0 | 0 | 0 | 22*01 | CAGALAFND | | | 6-4*01 | CASSPAVGT | 4-1*01 | CASSQEQ | 3190 |
| | | FML_V6L | | | | | | MRF | | | | GDEKLFF | | LSTYEQYF | 3688 |
| JH11 | Neo⁺WT⁻ | OR14C36- | IPO9- | 0 | 0 | 0 | 3*01 | CAVRDPYNF | | | | | | | 3191 |
| | | FML_V6L | FSS_E4D | | | | | NKFYF | | | | | | | |
| JH3 | Neo⁺WT⁻ | HERC1- | 0 | 0 | 0 | 0 | | | | | | | | | |
| | | SLL_PS | | | | | | | | | | | | | |
| JH7 | Neo⁺WT⁻ | A2ML1- | 0 | 0 | 0 | 0 | 27*01 | CAGARRDDK | | | 9*01 | CASSEPGPW | | | 3192 |
| | | YLD_K7R | | | | | | IIF | | | | AFF | | | 3689 |
| KA12 | Neo⁺WT⁻ | TRIM16- | 0 | 0 | 0 | 0 | 22*01 | CAVKTSYDK | | | 11-3*01 | CASSVTSDQ | | | 3193 |
| | | RMA_R1T | | | | | | VIF | | | | TQYF | | | 3690 |
| KB2 | Neo⁺WT⁻ | ATP6AP1- | 0 | 0 | 0 | 0 | 12-2*01 | CAVTTTSGG | | | | | | | 3194 |
| | | KLG_G3W | | | | | | YQKVTF | | | | | | | |
| KB9 | Neo⁺WT⁻ | CDC37L1- | 0 | 0 | 0 | 0 | 8-1*01 | CAVNAGNTG | | | 13*01 | CASSFRGNT | | | 3195 |
| | | FLS_P6L | | | | | | KLIF | | | | GELFF | | | 3691 |
| KC3 | Neo⁺WT⁻ | PHKA2- | 0 | 0 | 0 | 0 | | | | | | | | | |
| | | LLS_SF | | | | | | | | | | | | | |
| KC6 | Neo⁺WT⁻ | KCNB2- | 0 | 0 | 0 | 0 | 12-2*01 | CAVSNDYKL | | | 3-1*01 | CASSPTGTG | | | 3196 |
| | | LLA_P6T | | | | | | SF | | | | GSDTQYF | | | 3692 |
| KC8 | Neo⁺WT⁻ | HAUS3- | 0 | 0 | 0 | 0 | 12-2*01 | CAVQGGGA | | | 13*01 | CASSFMTEA | | | 3197 |
| | | ILN_T7A | | | | | | DGLTF | | | | GELFF | | | 3693 |
| KD4 | Neo⁺WT⁻ | MRM1- | 0 | 0 | 0 | 0 | 8-1*01 | CAVIANNND | | | 19*01 | CASDSGSGQ | | | 3198 |
| | | 9_T6P | | | | | | MRF | | | | PQHF | | | 3694 |
| KD7 | Neo⁺WT⁻ | GLRA1- | KCNB2- | 0 | 0 | 0 | | | | | 6-5*01 | CASFNTGEL | | | 3695 |
| | | LIF_F6L | LLA_P6T | | | | | | | | | FF | | | |
| KE1 | Neo⁺WT⁻ | PRSS16- | 0 | 0 | 0 | 0 | | | | | 20-1*01 | CSARDPVGG | | | 3696 |
| | | LLL_L1Q | | | | | | | | | | SNTGELFF | | | |
| KE10 | Neo⁺WT⁻ | HAUS3- | 0 | 0 | 0 | 0 | 22*01F | QGGKLIF | 14/ | CAIPPSGT | | | | | 3199 |
| | | ILN_T7A | | | | | | | DV4*01 | YKYIF | | | | | 3467 |
| KE11 | Neo⁺WT⁻ | ZDHHC7- | 0 | 0 | 0 | 0 | 1-1*01 | CAAWNTGF | | | | | | | 3200 |
| | | SLL_P7L | | | | | | QKLVF | | | | | | | |
| KE2 | Neo⁺WT⁻ | ITIH6- | 0 | 0 | 0 | 0 | | | | | | | | | |
| | | RLG_G3V | | | | | | | | | | | | | |
| KE6 | Neo⁺WT⁻ | DCHS1- | PELP1- | 0 | 0 | 0 | 12-2*01 | CAVNVNDYK | | | 9*01 | CASSPTAEA | | | 3201 |
| | | TLF_I5M | LVL_L3F | | | | | LSF | | | | FF | | | 3697 |
| KF3 | Neo⁺WT⁻ | C17orf75- | 0 | 0 | 0 | 0 | | | | | | | | | |
| | | ALS_V7A | | | | | | | | | | | | | |
| KF5 | Neo⁺WT⁻ | 0 | 0 | 0 | 0 | 0 | 13-1*01 | CAASWEQG | | | 3-1*01 | CASSQDRGR | | | 3202 |
| | | | | | | | | SNYKLTF | | | | DQETQYF | | | 3698 |
| KF6 | Neo⁺WT⁻ | INTS1- | 0 | 0 | 0 | 0 | 39*01 | CAPSAGGGS | | | 2*01 | CASSPLGLA | | | 3203 |
| | | VLL_L3F | | | | | | EKLVF | | | | EQETQYF | | | 3699 |
| KG10 | Neo⁺WT⁻ | KCNB2- | MAR11- | 0 | 0 | 0 | 9-2 | CALSDPGFG | | | 7-9*01 | CASSLVRDR | | | 3204 |
| | | LLA_P6T | 9_F1L | | | | | NVLHC | | | | HTEAFF | | | 3700 |
| KG11 | Neo⁺WT⁻ | DHX33- | 0 | 0 | 0 | 0 | 29/ | YQLTF | 8-6*01 | CAVIDPAR | | | | | 3205 |
| | | LLA_K5T | | | | | DV5*01F | | | ARLMF | | | | | 3468 |
| KG12 | Neo⁺WT⁻ | C3orf58- | ST6GALNAC2- | 0 | 0 | 0 | | | | | 7-9*01 | CASGGDAYE | | | 3701 |
| | | LMV_L4P | LLF_Y6H | | | | | | | | | QYF | | | |
| KG6 | Neo⁺WT⁻ | GOLGA3- | 0 | 0 | 0 | 0 | 21*01 | CAVRSYNTD | | | 6-6*01 | CASTPGTSA | 7-3*01 | RASSFTAP | 3206 |
| | | SLD_P4L | | | | | | KLIF | | | | SRDTQYF | | GLQYNEQ | 3702 |
| | | | | | | | | | | | | | | FF | |
| KH11 | Neo⁺WT⁻ | ATP6AP1- | 0 | 0 | 0 | 0 | 8-3*01 | CAVDETTDS | | | 4-1*01 | CASSPGTAY | | | 3207 |
| | | KLG_G3W | | | | | | WGKLQF | | | | EQYF | | | 3703 |
| KH6 | Neo⁺WT⁻ | DRAM1- | 0 | 0 | 0 | 0 | | | | | | | | | |
| | | FII_I3F | | | | | | | | | | | | | |
| KH7 | Neo⁺WT⁻ | 0 | 0 | 0 | 0 | 0 | 8-3*01 | CAHLSGGYN | | | 13*01 | CASSLSADT | | | 3208 |
| | | | | | | | | KLIF | | | | QYF | | | 3704 |
| LA3 | Neo⁺WT⁻ | LCP1- | 0 | 0 | 0 | 0 | 17*01 | CATDANNAG | | | 2*01 | CASSDGNEQ | | | 3209 |
| | | NLF_PL | | | | | | NMLTF | | | | FF | | | 3705 |
| LA6 | Neo⁺WT⁻ | OR9Q2- | 0 | 0 | 0 | 0 | 19*01 | CALSEEADN | 36/ | CAVGRYD | 6-1*01 | CASDSNYGY | | | 3210 |
| | | SID_S1F | | | | | | NDMRF | DV7*01 | YKLSF | | TF | | | 3469 |
| | | | | | | | | | | | | | | | 3706 |
| LB2 | Neo⁺WT⁻ | ATP6AP1- | 0 | 0 | 0 | 0 | 38-2/ | CAYRRMVS | 22*01 | CAVGSQG | 4-1*01 | CASSPGTGY | | | 3211 |
| | | KLG_G3W | | | | | DV8*01 | GGSNYKLTF | | GSEKLVF | | EQYF | | | 3470 |
| | | | | | | | | | | | | | | | 3707 |
| LB5 | Neo⁺WT⁻ | 0 | 0 | 0 | 0 | 0 | 38-2/ | CAYREGAQK | | | | | | | 3212 |
| | | | | | | | DV8*01 | LVF | | | | | | | |
| LB7 | Neo⁺WT⁻ | ATP6AP1- | 0 | 0 | 0 | 0 | | | | | 9*01 | CASSVAGGY | | | 3708 |
| | | KLG_G3W | | | | | | | | | | EQYF | | | |
| LC4 | Neo⁺WT⁻ | A2ML1- | 0 | 0 | 0 | 0 | 8-3*01 | CAVGSPDYK | | | 7-9*01 | CASSWDRG | | | 3213 |
| | | YLD_K7R | | | | | | LSF | | | | TYEQYF | | | 3709 |
| LD12 | Neo⁺WT⁻ | GANAB- | 0 | 0 | 0 | 0 | 39*01 | CAVVQTSGS | | | 13*01 | CASSWRRG | | | 3214 |
| | | ALY_S5F | | | | | | RLTF | | | | TDTQYF | | | 3710 |
| LD2 | Neo⁺WT⁻ | VN1R2- | 0 | 0 | 0 | 0 | 17*01 | CATDAWGH | | | 5-4*01 | CASSLEFGA | | | 3215 |
| | | LML_L3F | | | | | | GGSQGNLIF | | | | DTQYF | | | 3711 |
| LD5 | Neo⁺WT⁻ | HAUS3- | 0 | 0 | 0 | 0 | | | | | | | | | |
| | | ILN_T7A | | | | | | | | | | | | | |
| LD6 | Neo⁺WT⁻ | PIGN- | 0 | 0 | 0 | 0 | 38-2/ | CAYRSDGD | | | 6-1*01 | CASSRTGSL | | | 3216 |
| | | FLT_P7H | | | | | DV8*01 | MRF | | | | NYGYTF | | | 3712 |
| LE12 | Neo⁺WT⁻ | TEAD1- | 0 | 0 | 0 | 0 | 3*01 | CAVRDGGSA | | | 11-2*01 | CASSSQELT | | | 3217 |
| | | VLE_L8F | | | | | | SKIIF | | | | EAFF | | | 3713 |
| LE4 | Neo⁺WT⁻ | 0 | 0 | 0 | 0 | 0 | 14/ | CAMRERGY | | | | | | | 3218 |
| | | | | | | | DV4*01 | STLTF | | | | | | | |
| LE6 | Neo⁺WT⁻ | CLCN4- | CLCN4- | 0 | 0 | 0 | 12-3*01 | CAMSLSNFG | | | 6-1*01 | CASSEKPDT | | | 3219 |
| | | LLA_G8V | LLA | | | | | NEKLTF | | | | QYF | | | 3714 |
| LE8 | Neo⁺WT⁻ | ATP6AP1- | 0 | 0 | 0 | 0 | 22*01 | CAVVKTSYD | | | 4-1*01 | CASSPGQGY | | | 3220 |
| | | KLG_G3W | | | | | | KVIF | | | | EQYF | | | 3715 |
| LF7 | Neo⁺WT⁻ | HAUS3- | 0 | 0 | 0 | 0 | | | | | | | | | |
| | | ILN_T7A | | | | | | | | | | | | | |
| LG11 | Neo⁺WT⁻ | PIGN- | 0 | 0 | 0 | 0 | 12-2*01 | CAVPRNSGN | | | 13*01 | CASSTLIGSG | | | 3221 |
| | | FLT_P7H | | | | | | TPLVF | | | | NTIYF | | | 3716 |
| LG7 | Neo⁺WT⁻ | ATP6AP1- | 0 | 0 | 0 | 0 | 12-2*01 | CAVNDGTAS | | | 4-1*01 | CASSQVVVG | | | 3222 |
| | | KLG_G3W | | | | | | KLTF | | | | YGYTF | | | 3717 |
| LH1 | Neo⁺WT⁻ | USP28- | 0 | 0 | 0 | 0 | | | | | | | | | |
| | | LII_C5F | | | | | | | | | | | | | |
| LH3 | Neo⁺WT⁻ | ITIH6- | 0 | 0 | 0 | 0 | | | | | | | | | |
| | | RLG_G3V | | | | | | | | | | | | | |
| LH5 | Neo⁺WT⁻ | PIGN- | 0 | 0 | 0 | 0 | | | | | 20-1*01 | CSARTGIGP | | | 3718 |
| | | FLT_P7H | | | | | | | | | | YEQYF | | | |
| LH7 | Neo⁺WT⁻ | RYR3- | 0 | 0 | 0 | 0 | | | | | | | | | |
| | | VLN_E6K | | | | | | | | | | | | | |
| MA10 | Neo⁺WT⁻ | ATP6AP1- | 0 | 0 | 0 | 0 | 12-2*01 | CAVTVDDMR | | | 9*01 | CASSPAPAY | | | 3223 |
| | | KLG_G3W | | | | | | F | | | | EQYF | | | 3719 |
| MA4 | Neo⁺WT⁻ | TEAD1- | 0 | 0 | 0 | 0 | 3*01 | CAVSLLSGG | | | | | | | 3224 |
| | | SVL_L9F | | | | | | YNKLIF | | | | | | | |
| MA9 | Neo⁺WT⁻ | SMOX- | 0 | 0 | 0 | 0 | 12-2*01 | CAESLDTDK | | | 20-1*01 | CSARGGGFE | | | 3225 |
| | | KLA_KN | | | | | | LIF | | | | TQYF | | | 3720 |
| MB1 | Neo⁺WT⁻ | HAUS3- | DHX33- | 0 | 0 | 0 | 12-2*01 | CAVDNARLM | | | 19*01 | CASSMSGW | | | 3226 |
| | | ILN_T7A | LLA_M4I | | | | | F | | | | GDTQYF | | | 3721 |
| MB10 | Neo⁺WT⁻ | 0 | 0 | 0 | 0 | 0 | 12-2*01 | CAVNGGGS | | | | | | | 3227 |
| | | | | | | | | QGNLIF | | | | | | | |
| MB8 | Neo⁺WT⁻ | C17orf75- | 0 | 0 | 0 | 0 | 19*01 | CALSEIVPTS | | | 5-4*01 | CASSSPSGY | | | 3228 |
| | | ALS_V7A | | | | | | GTYKYIF | | | | EQYF | | | 3722 |
| MB9 | Neo⁺WT⁻ | INTS1- | 0 | 0 | 0 | 0 | 4*01 | CLVGDSWN | | | 20-1*01 | CSARWDRV | | | 3229 |
| | | VLL_L3F | | | | | | YGQNFVF | | | | SSSTDTQYF | | | 3723 |
| MC10 | Neo⁺WT⁻ | OR14C36- | 0 | 0 | 0 | 0 | 14/ | CAMGSGYAL | | | | | | | 3230 |
| | | FML_V6L | | | | | DV4*01 | NF | | | | | | | |
| MC12 | Neo⁺WT⁻ | SLC16A7- | HTR1F- | 0 | 0 | 0 | 1-2*01 | CAVRDYGQK | | | 4-1*01 | CASSPTPGT | | | 3231 |
| | | AMA_P6L | 9_V1M | | | | | LLF | | | | GETQYF | | | 3724 |
| MC4 | Neo⁺WT⁻ | HAUS3- | 0 | 0 | 0 | 0 | 12-2*01 | CAVTPGTALI | | | 6-5*01 | CASSRDGPS | | | 3232 |
| | | ILN_T7A | | | | | | F | | | | SYEQYF | | | 3725 |
| MC7 | Neo⁺WT⁻ | INTS1- | 0 | 0 | 0 | 0 | 21*01 | CAVKGNDM | | | 10-2*01 | CASSEGWV | | | 3233 |
| | | VLL_L3F | | | | | | RF | | | | DTQYF | | | 3726 |
| MC8 | Neo⁺WT⁻ | ATP6AP1- | 0 | 0 | 0 | 0 | 8-3*01 | CAVFMEYGN | | | 4-1*01 | CASSQATGY | | | 3234 |
| | | KLG_G3W | | | | | | KLVF | | | | EQYF | | | 3727 |
| MD1 | Neo⁺WT⁻ | ATP6AP1- | 0 | 0 | 0 | 0 | | | | | 9*01 | CASSPSGGV | | | 3728 |
| | | KLG_G3W | | | | | | | | | | YGYTF | | | |
| MD11 | Neo⁺WT⁻ | OR5M3- | 0 | 0 | 0 | 0 | | | | | 3-1*01 | CASSPPDGQ | | | 3729 |
| | | KMV_T8N | | | | | | | | | | GDYGYTF | | | |
| MD12 | Neo⁺WT⁻ | OR14C36- | 0 | 0 | 0 | 0 | | | | | 27*01 | CASSSLGGY | | | 3730 |
| | | FML_V6L | | | | | | | | | | EQYF | | | |
| MD7 | Neo⁺WT⁻ | ATP6AP1- | 0 | 0 | 0 | 0 | 30*01 | CGTGGAGD | | | 9*01 | CASSVSTNY | | | 3235 |
| | | KLG_G3W | | | | | | YKLSF | | | | EQYF | | | 3731 |
| MD9 | Neo⁺WT⁻ | ATP6AP1- | 0 | 0 | 0 | 0 | 6*01 | CAPFNTDKLI | | | 20-1*01 | CSARDVGIS | | | 3236 |
| | | KLG_G3W | | | | | | F | | | | YEQYF | | | 3732 |
| ME1 | Neo⁺WT⁻ | KCNB2- | 0 | 0 | 0 | 0 | 3*01 | CAVRVGGD | | | 28*01 | CASTVRQGS | | | 3237 |
| | | LLA_P6T | | | | | | MRF | | | | NQPQHF | | | 3733 |
| ME11 | Neo⁺WT⁻ | TRIM58- | ATP6AP1- | 0 | 0 | 0 | 12-2*01 | CAVDLEVGG | | | 4-1*01 | CASSPDRFY | | | 3238 |
| | | YMV_V3F | KLG_G3W | | | | | NKLVF | | | | EQYF | | | 3734 |
| ME12 | Neo⁺WT⁻ | INTS1- | 0 | 0 | 0 | 0 | 14/ | CAMRELLFG | | | 7-3*01 | CASSSPGQG | | | 3239 |
| | | VLL_L3F | | | | | DV4*01 | NEKLTF | | | | YYEQYF | | | 3735 |
| ME2 | Neo⁺WT⁻ | ATP6AP1- | 0 | 0 | 0 | 0 | | | | | | | | | |
| | | KLG_G3W | | | | | | | | | | | | | |
| ME4 | Neo⁺WT⁻ | SHROOM2- | 0 | 0 | 0 | 0 | 38-2/ | CAYSPYNNN | | | 6-5*01 | CASSYVNGG | | | 3240 |
| | | KLL_D6V | | | | | DV8*01 | DMRF | | | | AIGGELFF | | | 3736 |
| ME7 | Neo⁺WT⁻ | INTS1- | 0 | 0 | 0 | 0 | 12-3*01 | CASYSGGGA | | | 13*01 | CASSLGAGS | | | 3241 |
| | | VLL_L3F | | | | | | DGLTF | | | | YEQYF | | | 3737 |
| MF10 | Neo⁺WT⁻ | ITIH6- | 0 | 0 | 0 | 0 | 14/ | CAMREGPG | 29/ | CAAKWGN | 29-1*01 | CSVEEWDTS | | | 3242 |
| | | RLG_G3V | | | | | DV4*01 | NTPLVF | DV5*01 | NDMRF | | GNTIYF | | | 3471 |
| | | | | | | | | | | | | | | | 3738 |
| MF12 | Neo⁺WT⁻ | SSPN- | 0 | 0 | 0 | 0 | | | | | | | | | |
| | | LMA_S8F | | | | | | | | | | | | | |
| MF3 | Neo⁺WT⁻ | ATP6AP1- | 0 | 0 | 0 | 0 | | | | | 4-1*01 | CASSQGEGY | | | 3739 |
| | | KLG_G3W | | | | | | | | | | EQYF | | | |
| MF8 | Neo⁺WT⁻ | OR14C36- | 0 | 0 | 0 | 0 | 21*01 | CAVRPDGYA | | | 13*01 | CASNLGGDN | | | 3243 |
| | | FML_V6L | | | | | | LNF | | | | EQFF | | | 3740 |
| MF9 | Neo⁺WT⁻ | MLL2- | 0 | 0 | 0 | 0 | 8-6*01 | CAVISTGGT | | | 11-2*01 | CASSFSGTF | | | 3244 |
| | | ALS_L8H | | | | | | SYGKLTF | | | | EAFF | | | 3741 |
| MG11 | Neo⁺WT⁻ | ATP6AP1- | 0 | 0 | 0 | 0 | 41*01 | CAVGEDGQ | | | 4-1*01 | CASSPGQGY | | | 3245 |
| | | KLG_G3W | | | | | | NFVF | | | | EQYF | | | 3715 |
| MG5 | Neo⁺WT⁻ | ATP6AP1- | SLC2A4- | 0 | 0 | 0 | 12-2*01 | CAVAGVISG | | | 19*01 | CASSISPSSY | | | 3246 |
| | | KLG_G3W | ILI_A4T | | | | | TYKYIF | | | | EQYF | | | 3742 |
| MH1 | Neo⁺WT⁻ | VN1R5- | 0 | 0 | 0 | 0 | | | | | | | | | |
| | | MII_S7Y | | | | | | | | | | | | | |
| MH2 | Neo⁺WT⁻ | CD47- | 0 | 0 | 0 | 0 | 5*01 | CAERDGGFK | | | 13*01 | CASSPRTGF | | | 3247 |
| | | GLT_V6F | | | | | | TIF | | | | SSGNTIYF | | | 3743 |
| MH4 | Neo⁺WT⁻ | 0 | 0 | 0 | 0 | 0 | | | | | | | | | |
| MH6 | Neo⁺WT⁻ | OR10A3- | 0 | 0 | 0 | 0 | | | | | | | | | |
| | | ILI_V6F | | | | | | | | | | | | | |
| MH8 | Neo⁺WT⁻ | MRM1- | 0 | 0 | 0 | 0 | 20*01 | CAVIWYNNN | | | 6-5*01 | CASSYSGAE | | | 3248 |
| | | 9_T6P | | | | | | DMRF | | | | QYF | | | 3744 |
| NA12 | Neo⁺WT⁻ | SMARCD3- | 0 | 0 | 0 | 0 | 8-3*01 | CAVYSGGGA | | | | | | | 3075 |
| | | KLF_H8Y | | | | | | DGLTF | | | | | | | |
| NA8 | Neo⁺WT⁻ | VN1R5- | 0 | 0 | 0 | 0 | 19*01 | CALSDPLGR | | | 6-1*01 | CASSEFTRS | | | 3249 |
| | | MII_S7Y | | | | | | DDKIIF | | | | YEQYF | | | 3745 |
| NB1 | Neo⁺WT⁻ | MRM1- | 0 | 0 | 0 | 0 | 12-3*01 | CAPPRRDDK | 20*01 | CAVQGYS | 19*01 | CASSIAPGN | | | 3250 |
| | | 9_T6P | | | | | | IIF | | NDYKLSF | | EQYF | | | 3472 |
| | | | | | | | | | | | | | | | 3746 |
| NB10 | Neo⁺WT⁻ | 0 | 0 | 0 | 0 | 0 | 12-2*01 | CAVNRDDKII | | | 3-1*01 | CASSQYSLS | | | 3251 |
| | | | | | | | | F | | | | TDTQYF | | | 3748 |
| NB7 | Neo⁺WT⁻ | OR5M3- | 0 | 0 | 0 | 0 | 14/ | CAMGDNYG | | | 9*01 | CASSVVGAR | | | 3252 |
| | | KMV_T8N | | | | | DV4*01 | QNFVF | | | | TDTQYF | | | 3748 |
| NB9 | Neo⁺WT⁻ | TEAD1- | 0 | 0 | 0 | 0 | 14/ | CAMKGAGS | | | 2*01 | CASSDPRGQ | | | 3253 |
| | | SVL_L9F | | | | | DV4*01 | YQLTF | | | | PNQPQHF | | | 3749 |
| NC12 | Neo⁺WT⁻ | PHKA2- | 0 | 0 | 0 | 0 | 12-2*01 | CASRPDKLIF | | | 27*01 | CASSPGGYY | | | 3254 |
| | | LLS_SF | | | | | | | | | | GYTF | | | 3750 |
| NC2 | Neo⁺WT⁻ | DRAM1- | GCN1L1-9 | 0 | 0 | 0 | | | | | | | | | |
| | | FII_I3F | | | | | | | | | | | | | |
| NC3 | Neo⁺WT⁻ | OR14C36- | 0 | 0 | 0 | 0 | | | | | 7-9*01 | CASNTGYQE | | | 3751 |
| | | FML_V6L | | | | | | | | | | TQYF | | | |
| NC4 | Neo⁺WT⁻ | HAUS3- | 0 | 0 | 0 | 0 | 22*01 | CAVTDNYGQ | | | 6-5*01 | CASSYNQGY | | | 3255 |
| | | ILN_T7A | | | | | | NFVF | | | | EQYF | | | 3752 |
| ND4 | Neo⁺WT⁻ | PHKA2- | 0 | 0 | 0 | 0 | | | | | | | | | |
| | | LLS_SF | | | | | | | | | | | | | |
| NE2 | Neo⁺WT⁻ | 0 | 0 | 0 | 0 | 0 | 14/ | CAMREGDV | | | 9*01 | CASSVTPAD | | | 3256 |
| | | | | | | | DV4*01 | SF | | | | TQYF | | | 3753 |
| NE5 | Neo⁺WT⁻ | DHX33- | 0 | 0 | 0 | 0 | 12-2*01 | CALNNARLM | | | | | | | 3257 |
| | | LLA_M4I | | | | | | F | | | | | | | |
| NE6 | Neo⁺WT⁻ | ATP6AP1- | 0 | 0 | 0 | 0 | 12-2*01 | CAVNRDSGY | | | 4-1*01 | CASSLEDSA | | | 3258 |
| | | KLG_G3W | | | | | | ALNF | | | | NYGYTF | | | 3754 |
| NE9 | Neo⁺WT⁻ | GANAB- | 0 | 0 | 0 | 0 | 12-2*01 | CAVTTDSWG | | | 6-5*01 | CASSYSGQG | | | 3259 |
| | | ALY_S5F | | | | | | KLQF | | | | YTF | | | 3755 |
| NF3 | Neo⁺WT⁻ | RYR3- | 0 | 0 | 0 | 0 | | | | | | | | | |
| | | VLN_E6K | | | | | | | | | | | | | |
| NF6 | Neo⁺WT⁻ | COL18A1- | 0 | 0 | 0 | 0 | 4*01 | CLVARSYNN | | | 28*01 | CASSSGYNE | | | 3260 |
| | | VLL_S8F | | | | | | NDMRF | | | | QFF | | | 3756 |
| NF7 | Neo⁺WT⁻ | SREBF1- | 0 | 0 | 0 | 0 | 12-2*01 | CAVRGSGTY | | | 19*01 | CASSISTEAF | | | 3261 |
| | | YLQ_L6M | | | | | | KYIF | | | | F | | | 3757 |
| NF9 | Neo⁺WT⁻ | CD47- | 0 | 0 | 0 | 0 | 17*01 | CGAGNMLTF | 12-2*01 | CAVNTFTG | 28*01 | CASTKTGLG | | | 3262 |
| | | GLT_V6F | | | | | | | | GGNKLTF | | DQPQHF | | | 3473 |
| | | | | | | | | | | | | | | | 3758 |
| NG1 | Neo⁺WT⁻ | ATP6AP1- | 0 | 0 | 0 | 0 | 24*01 | CAFAGTYKYI | 8-6*01 | CAVKAGN | 9*01 | CASSVGGGE | | | 3263 |
| | | KLG_G3W | | | | | | F | | FGNEKLTF | | VEAFF | | | 3474 |
| | | | | | | | | | | | | | | | 3759 |
| NG11 | Neo⁺WT⁻ | HAUS3- | 0 | 0 | 0 | 0 | 38-2/ | CAYRTSYDK | | | 20-1*01 | CSAGIPGQV | | | 3264 |
| | | ILN_T7A | | | | | DV8*01 | VIF | | | | FSSNEKLFF | | | 3760 |
| NG6 | Neo⁺WT⁻ | TPP2- | 0 | 0 | 0 | 0 | | | | | | | | | |
| | | SLA_WL | | | | | | | | | | | | | |
| NG7 | Neo⁺WT⁻ | TRIM58- | 0 | 0 | 0 | 0 | 8-6*01 | CAAMGDSSY | | | 7-6*01 | CASSPYSGA | | | 3265 |
| | | YMV_V3F | | | | | | KLIF | | | | NVLTF | | | 3761 |
| NH1 | Neo⁺WT⁻ | ERBB2- | 0 | 0 | 0 | 0 | | | | | | | | | |
| | | ALI_H8Y | | | | | | | | | | | | | |
| NH12 | Neo⁺WT⁻ | NSDHL- | 0 | 0 | 0 | 0 | | | | | 9*01 | CASSLAGAD | | | 3762 |
| | | ILT_A9V | | | | | | | | | | NEQFF | | | |
| NH4 | Neo⁺WT⁻ | C3orf58- | 0 | 0 | 0 | 0 | 14/ | CAMSTLDQI | | | 2*01 | CASIPVGSR | | | 3266 |
| | | LMV_L4P | | | | | DV4*01 | QGAQKLVF | | | | NTIYF | | | 3763 |
| NH6 | Neo⁺WT⁻ | PELP1- | 0 | 0 | 0 | 0 | | | | | | | | | |
| | | RLH_L7F | | | | | | | | | | | | | |
| NH9 | Neo⁺WT⁻ | APBB2- | 0 | 0 | 0 | 0 | 12-2*01 | CAAAPNDYK | | | 28*01 | CASSLGQGY | | | 3267 |
| | | VQY_L7F | | | | | | LSF | | | | NEQFF | | | 3764 |
| OA6 | Neo⁺WT⁻ | DHX33- | TRIM16- | 0 | 0 | 0 | 12-2*01 | CAVNPGSQ | | | 3-1*01 | CASSQWGG | | | 3268 |
| | | LLA_K5T | RMA_R1T | | | | | GNLIF | | | | NEQFF | | | 3765 |
| OA8 | Neo⁺WT⁻ | HAUS3- | 0 | 0 | 0 | 0 | 26-2*01 | CILRDSSGG | | | 28*01 | CASAPGLNY | | | 3269 |
| | | ILN_T7A | | | | | | GADGLTF | | | | EQYF | | | 3766 |
| OB12 | Neo⁺WT⁻ | INTS1- | 0 | 0 | 0 | 0 | 39*01 | CAVDMRADS | | | | | | | 3270 |
| | | VLL_L3F | | | | | | NYQLIW | | | | | | | |
| OB9 | Neo⁺WT⁻ | 0 | 0 | 0 | 0 | 0 | | | | | | | | | |
| OC12 | Neo⁺WT⁻ | TRPV3- | 0 | 0 | 0 | 0 | 35*01 | CAGRSTGAG | | | 5-4*01 | CASSSESGE | | | 3271 |
| | | LLL_A8V | | | | | | SYQLTF | | | | LFF | | | 3767 |
| OC2 | Neo⁺WT⁻ | SMARCD3- | 0 | 0 | 0 | 0 | | | | | | | | | |
| | | KLF_H8Y | | | | | | | | | | | | | |
| OD10 | Neo⁺WT⁻ | SHROOM2- | 0 | 0 | 0 | 0 | 9-2*01 | CALSDRGAQ | | | | | | | 3272 |
| | | KLL_D6V | | | | | | KLVF | | | | | | | |
| OD2 | Neo⁺WT⁻ | OR5M3- | 0 | 0 | 0 | 0 | 3*01 | CAVSPLDGY | | | 2*01 | CASSEHRDH | | | 3273 |
| | | KMV_T8N | | | | | | NKLIF | | | | EQFF | | | 3768 |
| OD5 | Neo⁺WT⁻ | 0 | 0 | 0 | 0 | 0 | | | | | | | | | |
| OE11 | Neo⁺WT⁻ | PHKA2- | 0 | 0 | 0 | 0 | 12-2 | PYSSASKIIF | | | 6-1*01 | CASSVPGQG | | | 3274 |
| | | LLS_SF | | | | | | | | | | VLEQYF | | | 3769 |
| OE5 | Neo⁺WT⁻ | 0 | 0 | 0 | 0 | 0 | | | | | | | | | |
| OE7 | Neo⁺WT⁻ | 0 | 0 | 0 | 0 | 0 | 26-1*01 | CIVRLSNTG | | | 20-1*01 | CSARDRGSS | | | 3275 |
| | | | | | | | | NQFYF | | | | NEKLFF | | | 3770 |
| OF1 | Neo⁺WT⁻ | IGF1- | 0 | 0 | 0 | 0 | 23/ | CAARDPYNQ | | | 11-2*01 | CASSPDPSG | | | 3276 |
| | | TMS_S4F | | | | | DV6*01 | GGKLIF | | | | NEQFF | | | 3771 |
| OF2 | Neo⁺WT⁻ | 0 | 0 | 0 | 0 | 0 | | | | | | | | | |
| OF3 | Neo⁺WT⁻ | APBB2- | 0 | 0 | 0 | 0 | | | | | | | | | |
| | | VQY_L7F | | | | | | | | | | | | | |
| OG12 | Neo⁺WT⁻ | GANAB- | 0 | 0 | 0 | 0 | 8-3*01 | CAVVLTDSW | | | | | | | 3277 |
| | | ALY_S5F | | | | | | GKLQF | | | | | | | |
| OG2 | Neo⁺WT⁻ | 0 | 0 | 0 | 0 | 0 | | | | | | | | | |
| OH3 | Neo⁺WT⁻ | 0 | 0 | 0 | 0 | 0 | | | | | | | | | |
| OH6 | Neo⁺WT⁻ | VN1R2- | 0 | 0 | 0 | 0 | | | | | | | | | |
| | | LML_L3F | | | | | | | | | | | | | |
| SA5 | Neo⁺WT⁻ | OR10A3- | 0 | 0 | 0 | 0 | | | | | | | | | |
| | | ILI_V6F | | | | | | | | | | | | | |
| SA7 | Neo⁺WT⁻ | MPV17- | ITIH6- | 0 | 0 | 0 | 21*01 | CAVRPYDKV | | | 6-6*01 | CASSYGLEQ | | | 3278 |
| | | YLW_A5P | RLG_G3V | | | | | IF | | | | YF | | | 3772 |
| SA9 | Neo⁺WT⁻ | 0 | 0 | 0 | 0 | 0 | | | | | | | | | |
| SB8 | Neo⁺WT⁻ | ST6GALNAC2- | ST6GALNAC2- | 0 | 0 | 0 | 27*01 | CAGLDQPG | 12-2*01 | CAVNSGY | 5-4*01 | CASSLGQGT | | | 3279 |
| | | LLF_Y6H | LLF | | | | | GSYIPTF | | ALNF | | YEQYF | | | 3475 |
| | | | | | | | | | | | | | | | 3773 |
| SC3 | Neo⁺WT⁻ | HAUS3- | 0 | 0 | 0 | 0 | | | | | 5-6*01 | CASSSAGLP | | | 3774 |
| | | ILN_T7A | | | | | | | | | | EQYF | | | |
| SD5 | Neo⁺WT⁻ | DHX33- | 0 | 0 | 0 | 0 | | | | | 11-2*01 | CASSLDFQG | | | 3775 |
| | | LLA_K5T | | | | | | | | | | PRDF | | | |
| SD9 | Neo⁺WT⁻ | CD47- | 0 | 0 | 0 | 0 | | | | | 9*01 | CASSTGQGG | | | 3776 |
| | | GLT_V6F | | | | | | | | | | DTQYF | | | |
| SF9 | Neo⁺WT⁻ | 0 | 0 | 0 | 0 | 0 | | | | | | | | | |
| SG8 | Neo⁺WT⁻ | 0 | 0 | 0 | 0 | 0 | | | | | | | | | |
| SH12 | Neo⁺WT⁻ | 0 | 0 | 0 | 0 | 0 | | | | | | | | | |
| SH8 | Neo⁺WT⁻ | 0 | 0 | 0 | 0 | 0 | | | | | | | | | |
| GA11 | Neo⁻WT⁺ | HTR1F-10 | 0 | 0 | 0 | 0 | 10*01 | CVVSGGYQK | | | 6-6*01 | CASRRQATN | | | 3280 |
| | | | | | | | | VTF | | | | EKLFF | | | 3777 |
| GA3 | Neo⁻WT⁺ | 0 | 0 | 0 | 0 | 0 | | | | | 5-1*01 | CASSMDAYT | | | 3778 |
| | | | | | | | | | | | | EAFF | | | |
| GA4 | Neo⁻WT⁺ | OR5M3- | 0 | 0 | 0 | 0 | | | | | | | | | |
| | | KMV | | | | | | | | | | | | | |
| GA8 | Neo⁻WT⁺ | OR5M3- | 0 | 0 | 0 | 0 | 26-2*01 | CILNVPGGY | | | 7-9*01 | CASSSSGGL | | | 3281 |
| | | KMV | | | | | | QKVTF | | | | DTQYF | | | 3779 |
| GB10 | Neo⁻WT⁺ | SEC24A- | 0 | 0 | 0 | 0 | 29/ | CAASPATSG | | | 3-1*01 | CASSPRLAG | | | 3282 |
| | | FLY | | | | | DV5*01 | TYKYIF | | | | GKYNEQFF | | | 3780 |
| GB3 | Neo⁻WT⁺ | OR5M3- | 0 | 0 | 0 | 0 | 8-3*01 | CAVDRVTGG | | | | | | | 3283 |
| | | KMV | | | | | | GNKLTF | | | | | | | |
| GB5 | Neo⁻WT⁺ | 0 | 0 | 0 | 0 | 0 | | | | | 2*01 | CASSEERPG | | | 3781 |
| | | | | | | | | | | | | EGYTF | | | |
| GB6 | Neo⁻WT⁺ | ITIH6- | 0 | 0 | 0 | 0 | 4*01 | CLVVSNSSA | 1-1*01 | CAVSPGN | 19*01 | CASSIPSRTT | | | 3284 |
| | | RLG | | | | | | SKIIF | | TPLVF | | NYGYTF | | | 3476 |
| | | | | | | | | | | | | | | | 3782 |
| GC1 | Neo⁻WT⁺ | HTR1F-10 | 0 | 0 | 0 | 0 | 26-1*01 | CIVRAALYNN | | | | | | | 3285 |
| | | | | | | | | DMRF | | | | | | | |
| GC10 | Neo⁻WT⁺ | HTR1F-9 | 0 | 0 | 0 | 0 | 5*01 | CAETVNTGF | | | 2*01 | CARTGAGGN | | | 3286 |
| | | | | | | | | QKLVF | | | | TIYF | | | 3783 |
| GC11 | Neo⁻WT⁺ | 0 | 0 | 0 | 0 | 0 | 13-1*01 | CAANEKLVF | | | 19*01 | CASSIAPAYG | | | 3287 |
| | | | | | | | | | | | | YTF | | | 3784 |
| GC3 | Neo⁻WT⁺ | GCN1L1- | 0 | 0 | 0 | 0 | 12-2*01 | CAVKGMRF | | | 20-1*01 | CSARNRDTY | | | 3288 |
| | | 10 | | | | | | | | | | YNEQFF | | | 3785 |
| GC8 | Neo⁻WT⁺ | ITIH6- | ITIH6- | 0 | 0 | 0 | | | | | 28*01 | CASSFRRDT | | | 3786 |
| | | RLG | RLG_G3V | | | | | | | | | DTQYF | | | |
| GD12 | Neo⁻WT⁺ | RYR3- | 0 | 0 | 0 | 0 | 12-2*01 | CAGTHMRF | | | 7-9*01 | CASSSWTG | | | 3289 |
| | | VLN | | | | | | | | | | GNEQYF | | | 3787 |
| GD5 | Neo⁻WT⁺ | OR5M3- | 0 | 0 | 0 | 0 | | | | | | | | | |
| | | KMV | | | | | | | | | | | | | |
| GD9 | Neo⁻WT⁺ | PHKA2- | 0 | 0 | 0 | 0 | 5*01 | CAILPDSGA | | | 27*01 | CASSVPGTP | | | 3290 |
| | | LLS | | | | | | GSYQLTF | | | | NTEAFF | | | 3788 |
| GE10 | Neo⁻WT⁺ | OR5M3- | 0 | 0 | 0 | 0 | 34*01 | CGADNSGG | | | 7-9*01 | CASSLSWLD | | | 3291 |
| | | KMV | | | | | | GADGLTF | | | | SQETQYF | | | 3789 |
| GE12 | Neo⁻WT⁺ | SSPN-9 | 0 | 0 | 0 | 0 | 17*01 | CATDALSGT | | | 9*01 | CASSVDGTE | | | 3292 |
| | | | | | | | | YKYIF | | | | ETQYF | | | 3790 |
| GE7 | Neo⁻WT⁺ | ITIH6- | 0 | 0 | 0 | 0 | | | | | | | | | |
| | | RLG | | | | | | | | | | | | | |
| GE8 | Neo⁻WT⁺ | 0 | 0 | 0 | 0 | 0 | 14/ | CAMRESYNN | 38-2/ | CAYRSFSN | | | | | 3293 |
| | | | | | | | DV4*01 | NDMRF | DV8*01 | AGNNRKLI | | | | | 3477 |
| | | | | | | | | | | W | | | | | |
| GF8 | Neo⁻WT⁺ | 0 | 0 | 0 | 0 | 0 | | | | | | | | | |
| GG1 | Neo⁻WT⁺ | TBX3- | 0 | 0 | 0 | 0 | | | | | | | | | |
| | | GMG | | | | | | | | | | | | | |
| GG12 | Neo⁻WT⁺ | 0 | 0 | 0 | 0 | 0 | | | | | 7-9*01 | CASSLGGGI | | | 3791 |
| | | | | | | | | | | | | EAFF | | | |
| GG3 | Neo⁻WT⁺ | SHROOM2- | 0 | 0 | 0 | 0 | | | | | | | | | |
| | | KLL | | | | | | | | | | | | | |
| GG4 | Neo⁻WT⁺ | 0 | 0 | 0 | 0 | 0 | | | | | | | | | |
| GG7 | Neo⁻WT⁺ | LCP1- | 0 | 0 | 0 | 0 | 14/ | CALNNAGNM | | | 9*01 | CASSEWDTE | | | 3294 |
| | | NLF | | | | | DV4*01 | LTF | | | | AFF | | | 3792 |
| IA12 | Neo⁻WT⁺ | HOXC9- | 0 | 0 | 0 | 0 | 12-2*01 | CAVINSGAG | | | | | | | 3295 |
| | | YMY | | | | | | SYQLTF | | | | | | | |
| IA8 | Neo⁻WT⁺ | ITIH6- | 0 | 0 | 0 | 0 | 38-2/ | CAYRTQKLV | | | 6-5*01 | CASSAGTIYN | | | 3296 |
| | | RLG | | | | | DV8*01 | F | | | | EQFF | | | 3793 |
| IB10 | Neo⁻WT⁺ | OR5M3- | 0 | 0 | 0 | 0 | 19*01 | CALILTQGGS | | | 13*01 | CASSQVRDR | | | 3297 |
| | | KMV | | | | | | EKLVF | | | | DINYGYTF | | | 3794 |
| IB7 | Neo⁻WT⁺ | HAUS3- | 0 | 0 | 0 | 0 | 14/ | CARITGGGN | | | 2*01 | CASSGPRGY | | | 3298 |
| | | ILN | | | | | DV4*01 | KLTF | | | | TF | | | 3795 |
| IC11 | Neo⁻WT⁺ | HTR1F-10 | 0 | 0 | 0 | 0 | | | | | | | | | |
| IC2 | Neo⁻WT⁺ | OR5M3- | 0 | 0 | 0 | 0 | | | | | | | | | |
| | | KMV | | | | | | | | | | | | | |
| IC8 | Neo⁻WT⁺ | VN1R2- | 0 | 0 | 0 | 0 | | | | | 3-1*01 | CASSQDWG | | | 3796 |
| | | LML | | | | | | | | | | AEAFF | | | |
| IC9 | Neo⁻WT⁺ | 0 | 0 | 0 | 0 | 0 | 8-1*01 | CAVNALYNF | | | | | | | 3299 |
| | | | | | | | | NKFYF | | | | | | | |
| ID11 | Neo⁻WT⁺ | 0 | 0 | 0 | 0 | 0 | | | | | | | | | |
| ID2 | Neo⁻WT⁺ | ZDHHC7- | 0 | 0 | 0 | 0 | | | | | | | | | |
| | | SLL | | | | | | | | | | | | | |
| ID3 | Neo⁻WT⁺ | 0 | 0 | 0 | 0 | 0 | | | | | 7-9*01 | CASSLVLYD | | | 3797 |
| | | | | | | | | | | | | GGLQETQYF | | | |
| ID5 | Neo⁻WT⁺ | OR10A3- | 0 | 0 | 0 | 0 | | | | | 6-1*01 | CASSAFGIVA | | | 3798 |
| | | ILI | | | | | | | | | | DTQYF | | | |
| IE10 | Neo⁻WT⁺ | IPO9- | 0 | 0 | 0 | 0 | | | | | | | | | |
| | | FSS | | | | | | | | | | | | | |
| IE11 | Neo⁻WT⁺ | GPR174- | 0 | 0 | 0 | 0 | | | | | | | | | |
| | | FSF | | | | | | | | | | | | | |
| IE4 | Neo⁻WT⁺ | GLRA1- | 0 | 0 | 0 | 0 | 38-1*01 | CAYGTGANN | | | 15*01 | CATSGGQSN | | | 3300 |
| | | LIF | | | | | | LFF | | | | EKLFF | | | 3799 |
| IE6 | Neo⁻WT⁺ | 0 | 0 | 0 | 0 | 0 | | | | | | | | | |
| IE8 | Neo⁻WT⁺ | 0 | 0 | 0 | 0 | 0 | | | | | | | | | |
| IE9 | Neo⁻WT⁺ | OR5M3- | 0 | 0 | 0 | 0 | 8-6*01 | CAVSADKLIF | | | | | | | 3301 |
| | | KMV | | | | | | | | | | | | | |
| IF10 | Neo⁻WT⁺ | HERC1- | 0 | 0 | 0 | 0 | 14/ | CAMRAITQG | | | 28*01 | CASSLSYTP | | | 3302 |
| | | SLL | | | | | DV4*01 | GSERLVF | | | | HQPQHF | | | 3800 |
| IF12 | Neo⁻WT⁺ | OR5M3- | 0 | 0 | 0 | 0 | | | | | | | | | |
| | | KMV | | | | | | | | | | | | | |
| IF7 | Neo⁻WT⁺ | ITIH6- | 0 | 0 | 0 | 0 | | | | | | | | | |
| | | RLG | | | | | | | | | | | | | |
| IG1 | Neo⁻WT⁺ | GLRA1- | 0 | 0 | 0 | 0 | | | | | | | | | |
| | | LIF | | | | | | | | | | | | | |
| IG10 | Neo⁻WT⁺ | GLRA1- | 0 | 0 | 0 | 0 | 17*01 | CATDQGNTP | | | 13*01 | CASSPGGTN | | | 3303 |
| | | LIF | | | | | | LVF | | | | EKLFF | | | 3801 |
| IG12 | Neo⁻WT⁺ | 0 | 0 | 0 | 0 | 0 | 38-2/ | CAYIGYDMR | | | 6-6*01 | CASSYLMGQ | | | 3304 |
| | | | | | | | DV8*01 | F | | | | GKGQAFF | | | 3802 |
| IG3 | Neo⁻WT⁺ | OR5M3- | 0 | 0 | 0 | 0 | | | | | | | | | |
| | | KMV | | | | | | | | | | | | | |
| IG4 | Neo⁻WT⁺ | 0 | 0 | 0 | 0 | 0 | 17*01 | CAIADSWGK | | | | | | | 3305 |
| | | | | | | | | LQF | | | | | | | |
| IG5 | Neo⁻WT⁺ | OR5M3- | 0 | 0 | 0 | 0 | 19*01 | CALSEQTSY | | | 7-9*01 | CASSAGGTE | | | 3306 |
| | | KMV | | | | | | DKVIF | | | | AFF | | | 3803 |
| IH11 | Neo⁻WT⁺ | LCP1- | 0 | 0 | 0 | 0 | | | | | | | | | |
| | | NLF | | | | | | | | | | | | | |
| JA10 | Neo⁻WT⁺ | 0 | 0 | 0 | 0 | 0 | 41*01 | CAPTRNAGG | | | 4-1*01 | CASSPYGDQ | | | 3307 |
| | | | | | | | | TSYGKLTF | | | | LNTGELFF | | | 3804 |
| JA3 | Neo⁻WT⁺ | HTR1F-10 | 0 | 0 | 0 | 0 | 10*01 | CVVKGGYNK | | | 6-6*01 | CASNREVST | | | 3308 |
| | | | | | | | | LIF | | | | DTQYF | | | 3805 |
| JA8 | Neo⁻WT⁺ | 0 | 0 | 0 | 0 | 0 | 12-2*01 | CAVVHGGQ | | | | | | | 3309 |
| | | | | | | | | NFVF | | | | | | | |
| JB11 | Neo⁻WT⁺ | KAT6A- | 0 | 0 | 0 | 0 | 38-2/ | CAMEGNEKL | | | 12-3*01, | CASRGTGTG | | | 3310 |
| | | KLS | | | | | DV8*01 | TF | | | 12-4*01 | SYEQYF | | | 3806 |
| JB12 | Neo⁻WT⁺ | GLRA1- | 0 | 0 | 0 | 0 | 24*01 | CAPHSNYQL | | | | | | | 3311 |
| | | LIF | | | | | | IW | | | | | | | |
| JB4 | Neo⁻WT⁺ | OR8D4-10 | 0 | 0 | 0 | 0 | 8-3*01 | CAVAPGSGG | | | 29-1*01 | CSVPGTAYE | | | 3312 |
| | | | | | | | | SNYKLTF | | | | QYF | | | 3807 |
| JB7 | Neo⁻WT⁺ | OR5M3- | 0 | 0 | 0 | 0 | 14/ | CAMREVYNN | | | 10-3*01 | CAISDLDSN | | | 3313 |
| | | KMV | | | | | DV4*01 | AGNMLTF | | | | QPQHF | | | 3808 |
| JB8 | Neo⁻WT⁺ | ITIH6- | 0 | 0 | 0 | 0 | 19*01 | CALSGYSTL | | | 19*01 | CASSISGGS | | | 3314 |
| | | RLG | | | | | | TF | | | | YEQYF | | | 3809 |
| JB9 | Neo⁻WT⁺ | OR5M3- | 0 | 0 | 0 | 0 | 41*01 | CAAENRDDK | | | | | | | 3315 |
| | | KMV | | | | | | IIF | | | | | | | |
| JC1 | Neo⁻WT⁺ | OR5M3- | 0 | 0 | 0 | 0 | 19*01 | CALKGNNRL | 17*01 | CATEGSYI | 7-9*01 | CASSLSWED | | | 3316 |
| | | KMV | | | | | | AF | | PTF | | ENTDTQYF | | | 3478 |
| | | | | | | | | | | | | | | | 3810 |
| JC10 | Neo⁻WT⁺ | CNKSR1- | CNKSR1- | 0 | 0 | 0 | 3*01 | CDPIPTRRLS | | | | | | | 3317 |
| | | SLA | SLA_A9V | | | | | F | | | | | | | |
| JC3 | Neo⁻WT⁺ | 0 | 0 | 0 | 0 | 0 | 12-3*01 | CAMSVGNA | | | | | | | 3318 |
| | | | | | | | | GNMLTF | | | | | | | |
| JC4 | Neo⁻WT⁺ | 0 | 0 | 0 | 0 | 0 | 10*01 | CLVSGGYNK | | | 20-1*01 | CSARVPTSF | | | 3319 |
| | | | | | | | | LIF | | | | TDTQYF | | | 3811 |
| JC5 | Neo⁻WT⁺ | ITIH6- | 0 | 0 | 0 | 0 | 14/ | CAMRGYQK | | | 19*01 | CASSASEPS | | | 3320 |
| | | RLG | | | | | DV4*01 | VTF | | | | GETQYF | | | 3812 |
| JC6 | Neo⁻WT⁺ | OR5M3- | 0 | 0 | 0 | 0 | 19*01 | CALSEASEY | | | 7-9*01 | CASSFPVSD | | | 3321 |
| | | KMV | | | | | | GNKLVF | | | | PSTDTQYF | | | 3813 |
| JC9 | Neo⁻WT⁺ | 0 | 0 | 0 | 0 | 0 | 17*01 | CATEVQGAQ | | | 13*01 | CASSFGETQ | | | 3322 |
| | | | | | | | | KLVF | | | | YF | | | 3814 |
| JD1 | Neo⁻WT⁺ | CHD8- | 0 | 0 | 0 | 0 | 17*01 | CATDAEGAQ | | | 4-2*01 | CASSPTSGG | | | 3323 |
| | | KLN | | | | | | KLVF | | | | YEQYF | | | 3815 |
| JD2 | Neo⁻WT⁺ | PLXNB1- | 0 | 0 | 0 | 0 | | | | | | | | | |
| | | VLF | | | | | | | | | | | | | |
| JD5 | Neo⁻WT⁺ | 0 | 0 | 0 | 0 | 0 | 24*01 | CAFRFNKFY | | | 27*01 | CASGPNQPQ | | | 3324 |
| | | | | | | | | F | | | | HF | | | 3816 |
| JD6 | Neo⁻WT⁺ | 0 | 0 | 0 | 0 | 0 | 5*01 | CAVLDGYNK | | | | | | | 3325 |
| | | | | | | | | LIF | | | | | | | |
| JD7 | Neo⁻WT⁺ | 0 | 0 | 0 | 0 | 0 | 38-2/ | CAYRSAWD | | | 19*01 | CASSPWTGS | | | 3326 |
| | | | | | | | DV8*01 | MRF | | | | YQETQYF | | | 3817 |
| JE1 | Neo⁻WT⁺ | 0 | 0 | 0 | 0 | 0 | 38-2/ | CALSGGGAD | 38-2/ | CAYRSPFL | | | | | 3327 |
| | | | | | | | DV8*01 | GLTF | DV8*01 | RAGTASKL | | | | | 3479 |
| | | | | | | | | | | TF | | | | | |
| JE10 | Neo⁻WT⁺ | 0 | 0 | 0 | 0 | 0 | 10*01 | CVVSGGYNK | | | 2*01 | CARTGEDNS | | | 3328 |
| | | | | | | | | LIF | | | | PLHF | | | 3818 |
| JE5 | Neo⁻WT⁺ | OR5M3- | 0 | 0 | 0 | 0 | 12-2*01 | CAVNLYARL | | | 19*01 | CASSTGISYE | | | 3329 |
| | | KMV | | | | | | MF | | | | QYF | | | 3819 |
| JE6 | Neo⁻WT⁺ | 0 | 0 | 0 | 0 | 0 | 8-2*01 | CVDGGYQK | | | 6-1*01 | CASSEEVSD | | | 3330 |
| | | | | | | | | VTF | | | | DSPLHF | | | 3820 |
| JF10 | Neo⁻WT⁺ | PHKA2- | 0 | 0 | 0 | 0 | 12-2*01 | CAVKNDYKL | | | 5-6*01 | CASGRSGED | | | 3331 |
| | | LLS | | | | | | SF | | | | YGYTF | | | 3821 |
| JF2 | Neo⁻WT⁺ | OR5M3- | 0 | 0 | 0 | 0 | | | | | | | | | |
| | | KMV | | | | | | | | | | | | | |
| JF4 | Neo⁻WT⁺ | OR5M3- | 0 | 0 | 0 | 0 | 5*01 | CAEAISGGY | | | | | | | 3332 |
| | | KMV | | | | | | NKLIF | | | | | | | |
| JF8 | Neo⁻WT⁺ | CCM2- | 0 | 0 | 0 | 0 | | | | | | | | | |
| | | YML_R6H | | | | | | | | | | | | | |
| JG1 | Neo⁻WT⁺ | 0 | 0 | 0 | 0 | 0 | | | | | | | | | |
| JG10 | Neo⁻WT⁺ | 0 | 0 | 0 | 0 | 0 | | | | | | | | | |
| JG12 | Neo⁻WT⁺ | ZDHHC7- | 0 | 0 | 0 | 0 | 8-1*01 | CAVNKPNQA | | | 14*01 | CASSQNPGQ | | | 3333 |
| | | SLL | | | | | | GTALIF | | | | GIYSPLHF | | | 3822 |
| JG3 | Neo⁻WT⁺ | 0 | 0 | 0 | 0 | 0 | 12-2*01 | CAVKNTGFQ | | | | | | | 3334 |
| | | | | | | | | KLVF | | | | | | | |
| JG4 | Neo⁻WT⁺ | MLL2- | 0 | 0 | 0 | 0 | 1-2*01 | CAVSHLIAG | | | 9*01 | CASSGQGAY | | | 3335 |
| | | ALS | | | | | | GFKTIF | | | | ITDTQYF | | | 3823 |
| JG9 | Neo⁻WT⁺ | TTLL12- | 0 | 0 | 0 | 0 | 12-2*01 | CAVNEDKIIF | | | 12-3*01, | CASSLASGN | | | 3336 |
| | | KLP | | | | | | | | | 12-4*01 | EQFF | | | 3825 |
| JH10 | Neo⁻WT⁺ | 0 | 0 | 0 | 0 | 0 | 12-1*01 | CVVNGNNN | | | 19*01 | CASSKGGNQ | | | 3337 |
| | | | | | | | | DMRF | | | | PQHF | | | 3825 |
| JH12 | Neo⁻WT⁺ | 0 | 0 | 0 | 0 | 0 | 21*01 | CAVEGSNFG | | | | | | | 3338 |
| | | | | | | | | NEKLTF | | | | | | | |
| JH2 | Neo⁻WT⁺ | LCP1- | 0 | 0 | 0 | 0 | 12-2*01 | CAVSNNDM | | | 7-2*01 | CASSLAKMD | | | 3339 |
| | | NLF | | | | | | RF | | | | LPLAKNIQYF | | | 3826 |
| JH4 | Neo⁻WT⁺ | ZNF827- | 0 | 0 | 0 | 0 | | | | | | | | | |
| | | NLF | | | | | | | | | | | | | |
| JH5 | Neo⁻WT⁺ | APCDD1L- | 0 | 0 | 0 | 0 | | | | | | | | | |
| | | RLP | | | | | | | | | | | | | |
| JH8 | Neo⁻WT⁺ | 0 | 0 | 0 | 0 | 0 | | | | | | | | | |
| KA3 | Neo⁻WT⁺ | HAUS3- | 0 | 0 | 0 | 0 | 20*01 | CAVLLSNDY | 19*01 | CALSEGER | | | | | 3340 |
| | | ILN | | | | | | KLSF | | DDKIIF | | | | | 3480 |
| KA4 | Neo⁻WT⁺ | 0 | 0 | 0 | 0 | 0 | | | | | 4-2*01 | CASSQGDRD | | | 3827 |
| | | | | | | | | | | | | SGNTIYF | | | |
| KA5 | Neo⁻WT⁺ | LCP1- | 0 | 0 | 0 | 0 | 12-3*01 | CAMEDTNAG | | | 11-2*01 | CASSLGGDE | | | 3341 |
| | | NLF | | | | | | KSTF | | | | QYF | | | 3828 |
| KA7 | Neo⁻WT⁺ | OR5M3- | 0 | 0 | 0 | 0 | 9-2*01 | CALSDGEFY | | | | | | | 3342 |
| | | KMV | | | | | | NQGGKLIF | | | | | | | |
| KA8 | Neo⁻WT⁺ | OR5M3- | 0 | 0 | 0 | 0 | | | | | 7-9*01 | CASSMPTGT | | | 3829 |
| | | KMV | | | | | | | | | | DSYEQYF | | | |
| KA9 | Neo⁻WT⁺ | OR9Q2- | 0 | 0 | 0 | 0 | 12-1*01 | CVVILNARLM | | | 20-1*01 | CSAIVFSRG | | | 3343 |
| | | FLF | | | | | | F | | | | GDEQFF | | | 3830 |
| KB1 | Neo⁻WT⁺ | OR1G1- | 0 | 0 | 0 | 0 | 30*01 | CGTDNAGGT | | | 19*01 | CASSPGQGY | | | 3344 |
| | | FLF | | | | | | SYGKLTF | | | | EQYF | | | 3715 |
| KB10 | Neo⁻WT⁺ | CHD8- | 0 | 0 | 0 | 0 | 13-1*01 | CAASMGQA | | | 4-2*01 | CASSPAGTD | | | 3345 |
| | | KLN | | | | | | GTALIF | | | | YGYTF | | | 3831 |
| KB5 | Neo⁻WT⁺ | FNDC3B- | 0 | 0 | 0 | 0 | | | | | | | | | |
| | | VVL | | | | | | | | | | | | | |
| KB6 | Neo⁻WT⁺ | OR5M3- | 0 | 0 | 0 | 0 | | | | | 7-9*01 | CASSSINRD | | | 3832 |
| | | KMV | | | | | | | | | | KMNTEAFF | | | |
| KB8 | Neo⁻WT⁺ | GANAB- | 0 | 0 | 0 | 0 | 12-2*01 | CAVSGGGA | | | 5-6*01 | CASSPGTSY | | | 3346 |
| | | ALY | | | | | | DGLTF | | | | EQYF | | | 3833 |
| KC1 | Neo⁻WT⁺ | OR5M3- | 0 | 0 | 0 | 0 | 14/ | CAMREGRD | | | | | | | 3347 |
| | | KMV | | | | | DV4*01 | FGNEKLTF | | | | | | | |
| KC11 | Neo⁻WT⁺ | OR5M3- | 0 | 0 | 0 | 0 | 17*01 | CATDAGDDK | | | 7-9*01 | CASSLAVGQ | | | 3348 |
| | | KMV | | | | | | IIF | | | | PGEEEQYF | | | 3834 |
| KC2 | Neo⁻WT⁺ | OR9Q2- | 0 | 0 | 0 | 0 | 19*01 | CALSEWGS | | | | | | | 3349 |
| | | FLF | | | | | | QGNLIF | | | | | | | |
| KC5 | Neo⁻WT⁺ | DCHS1- | 0 | 0 | 0 | 0 | 14/ | CAMREGGD | 13-1*01 | CAAIIGQK | | | | | 3350 |
| | | TLF | | | | | DV4*01 | SSYKLIF | | LLF | | | | | 3481 |
| KC7 | Neo⁻WT⁺ | 0 | 0 | 0 | 0 | 0 | | | | | 12-3*01, | CASSKGAGV | | | 3835 |
| | | | | | | | | | | | 12-4*01 | FQETQYF | | | |
| KD11 | Neo⁻WT⁺ | OR5M3- | 0 | 0 | 0 | 0 | 19*01 | CALSEADDY | | | 20-1*01 | CSAHPRDVQ | | | 3351 |
| | | KMV | | | | | | KLSF | | | | ETQYF | | | 3836 |
| KD2 | Neo⁻WT⁺ | OR10A3- | 0 | 0 | 0 | 0 | | | | | | | | | |
| | | ILI | | | | | | | | | | | | | |
| KD6 | Neo⁻WT⁺ | 0 | 0 | 0 | 0 | 0 | | | | | 7-9*01 | CASSSTREQ | | | 3837 |
| | | | | | | | | | | | | LIGEKLFF | | | |
| KE4 | Neo⁻WT⁺ | OR5M3- | 0 | 0 | 0 | 0 | 8-1*01F | CAVKSGAGF | | | 7-9*01 | CASSLNRGL | | | 3352 |
| | | KMV | | | | | | GNVLHC | | | | NTGELFF | | | 3838 |
| KE5 | Neo⁻WT⁺ | 0 | 0 | 0 | 0 | 0 | 12-2*01 | CAVNWNYG | | | | | | | 3353 |
| | | | | | | | | GSQGNLIF | | | | | | | |
| KF10 | Neo⁻WT⁺ | 0 | 0 | 0 | 0 | 0 | | | | | 7-9*01 | CASSFSSLD | | | 3839 |
| | | | | | | | | | | | | NYGYTF | | | |
| KF7 | Neo⁻WT⁺ | SMOX- | 0 | 0 | 0 | 0 | 21*01 | CAVEPGDDY | | | 12-5*01 | CASDPDSLIH | | | 3354 |
| | | KLA | | | | | | KLSF | | | | NTGELFF | | | 3840 |
| KF8 | Neo⁻WT⁺ | OR5M3- | 0 | 0 | 0 | 0 | | | | | 7-9*01 | CASSSTGTG | | | 3841 |
| | | KMV | | | | | | | | | | GSYNSPLHF | | | |
| KF9 | Neo⁻WT⁺ | OR8D4-9 | OR8D4- | 0 | 0 | 0 | 23/ | CAVNQAGTA | | | 9*01 | CASSDNDW | | | 3355 |
| | | | 9_G3E | | | | DV6*01 | LIF | | | | RLQYF | | | 3842 |
| KG2 | Neo⁻WT⁺ | OR5M3- | 0 | 0 | 0 | 0 | | | | | 7-9*01 | CASSSPTSG | | | 3843 |
| | | KMV | | | | | | | | | | ADNEQFF | | | |
| KG3 | Neo⁻WT⁺ | 0 | 0 | 0 | 0 | 0 | 12-1*01 | CVVNLNYGG | | | 6-5*01 | CASSYSNGY | | | 3356 |
| | | | | | | | | SQGNLIF | | | | EQYF | | | 3844 |
| KG5 | Neo⁻WT⁺ | 0 | 0 | 0 | 0 | 0 | 5*01 | CAEGLEDTG | | | 19*01 | CASSPGGYG | | | 3357 |
| | | | | | | | | KLIF | | | | YTF | | | 3845 |
| KG8 | Neo⁻WT⁺ | 0 | 0 | 0 | 0 | 0 | | | | | | | | | |
| KH1 | Neo⁻WT⁺ | OR5M3- | 0 | 0 | 0 | 0 | 14/ | CAMREAHD | | | 7-9*01 | CASSFWGLP | | | 3358 |
| | | KMV | | | | | DV4*01 | NFGNEKLTF | | | | HQETQYF | | | 3846 |
| KH10 | Neo⁻WT⁺ | KAT6A- | 0 | 0 | 0 | 0 | 3*01 | CAVRDEDDK | | | 7-9*01 | CASSLASEQ | | | 3359 |
| | | KLS | | | | | | IIF | | | | YF | | | 3847 |
| KH12 | Neo⁻WT⁺ | OR5M3- | CLCN4- | 0 | 0 | 0 | 8-4*01 | CAVSARAFG | | | 7-9*01 | CASSADRTQ | | | 3360 |
| | | KMV | LLA | | | | | NEKLTF | | | | NYGYTF | | | 3848 |
| KH3 | Neo⁻WT⁺ | RYR3- | 0 | 0 | 0 | 0 | | | | | | | | | |
| | | VLN | | | | | | | | | | | | | |
| KH4 | Neo⁻WT⁺ | SEC24A- | 0 | 0 | 0 | 0 | 22*01 | CAVEDNFNK | | | | | | | 3361 |
| | | FLY | | | | | | FYF | | | | | | | |
| KH5 | Neo⁻WT⁺ | 0 | 0 | 0 | 0 | 0 | | | | | | | | | |
| KH8 | Neo⁻WT⁺ | 0 | 0 | 0 | 0 | 0 | 19*01 | CALSEAYSG | | | | | | | 3362 |
| | | | | | | | | SARQLTF | | | | | | | |
| LA10 | Neo⁻WT⁺ | 0 | 0 | 0 | 0 | 0 | 12-2*01 | CAVKSEYGN | | | 20-1*01 | CSAYPAGDG | | | 3363 |
| | | | | | | | | KLVF | | | | TGELFF | | | 3849 |
| LA11 | Neo⁻WT⁺ | OR5M3- | 0 | 0 | 0 | 0 | 19*01 | CALSEGNFG | | | 7-9*01 | CASSPPLWG | | | 3364 |
| | | KMV | | | | | | NEKLTF | | | | VYGYTF | | | 3850 |
| LA12 | Neo⁻WT⁺ | DCHS1- | 0 | 0 | 0 | 0 | 14/ | CAMRGGMD | | | | | | | 3365 |
| | | TLF | | | | | DV4*01 | SSYKLIF | | | | | | | |
| LA4 | Neo⁻WT⁺ | LCP1-NLF | 0 | 0 | 0 | 0 | 21*01 | CAVDGQAGT | 26-2*01 | CILRGIPR | 15*01 | CATSRVVTGN | | | 3366 |
| | | | | | | | | ALIF | | DSSYKLIF | | EQFF | | | 3482 |
| | | | | | | | | | | | | | | | 3851 |
| LA8 | Neo⁻WT⁺ | TBX3- | TBX3- | 0 | 0 | 0 | 14/ | CAMTSFQKL | | | 13*01 | CASSLRGEK | | | 3367 |
| | | GMG | GMG_T8M | | | | DV4*01 | VF | | | | NNYGYTF | | | 3852 |
| LA9 | Neo⁻WT⁺ | ITIH6- | 0 | 0 | 0 | 0 | 3*01 | CAVRDTRSY | | | 19*01 | CASSIQGNS | | | 3368 |
| | | RLG | | | | | | NTDKLIF | | | | NQPQHF | | | 3853 |
| LB1 | Neo⁻WT⁺ | OR5M3- | 0 | 0 | 0 | 0 | 26-1*01 | CIVRIIKAAG | 14/ | CAMREGRV | 7-9*01 | CASSLVRAD | | | 3370 |
| | | KMV | | | | | | NKLTF | DV4*01 | FGNEKLTF | | GETQYF | | | 3855 |
| LB11 | Neo⁻WT⁺ | ITIH6-RLG | 0 | 0 | 0 | 0 | 14/ | CAMRESNNA | | | 6-6*01 | CASSATGTV | | | 3371 |
| | | | | | | | DV4*01 | RLMF | | | | NTEAFF | | | 3856 |
| LB8 | Neo⁻WT⁺ | SEC24A- | 0 | 0 | 0 | 0 | 22*01 | CAVEMTTDS | | | 19*01 | CASSIGGYG | | | 3857 |
| | | FLY | | | | | | WGKLQF | | | | YTF | | | |
| LB9 | Neo⁻WT⁺ | 0 | 0 | 0 | 0 | 0 | | | | | 19*01 | CASTGTSYE | | | 3372 |
| | | | | | | | | | | | | QYF | | | 3858 |
| LC10 | Neo⁻WT⁺ | SEC24A- | 0 | 0 | 0 | 0 | 14/ | CAMRELYTG | | | 28*01 | CASSPSGTG | | | 3373 |
| | | FLY | | | | | DV4*01 | GFKTIF | | | | FYEQYF | | | 3859 |
| LC12 | Neo⁻WT⁺ | DOLPP1- | 0 | 0 | 0 | 0 | 8-2*01 | CGMDSSYKL | | | 20-1*01 | CSARVQGAY | | | |
| | | GLM | | | | | | IF | | | | EQYF | | | |
| LC2 | Neo⁻WT⁺ | LCP1- | 0 | 0 | 0 | 0 | 21 | CAVWVGFG | 19*01 | CALSRGG | 4-2*01 | CASSQVLGF | | | 3374 |
| | | NLF | | | | | | NVLHC | | GADGLTF | | SYEQYF | | | 3484 |
| | | | | | | | | | | | | | | | 3860 |
| LC7 | Neo⁻WT⁺ | ITIH6- | 0 | 0 | 0 | 0 | 29/ | CAGGDSWG | | | 4-1*01 | CASSRKGDS | | | 3375 |
| | | RLG | | | | | DV5*01 | KLQF | | | | PLHF | | | 3861 |
| LC8 | Neo⁻WT⁺ | SLC16A7- | 0 | 0 | 0 | 0 | | | | | 6-1*01 | CASSHDDRG | | | 3862 |
| | | AMA | | | | | | | | | | PNEKLFF | | | |
| LC9 | Neo⁻WT⁺ | KAT6A- | 0 | 0 | 0 | 0 | 12-2*01 | CAVSGDAGN | | | 9*01 | CASSTGGDT | | | 3376 |
| | | KLS | | | | | | MLTF | | | | QYF | | | 3863 |
| LD1 | Neo⁻WT⁺ | OR5M3- | 0 | 0 | 0 | 0 | 5*01 | CAESMGND | | | 6-2*01, | CASSYGHPG | | | 3377 |
| | | KMV | | | | | | MRF | | | 6-3*01 | EQYF | | | 3864 |
| LD3 | Neo⁻WT⁺ | ZDHHC7- | 0 | 0 | 0 | 0 | 12-2*01 | CAVNNARLM | | | 20-1*01 | CSALTGLGN | | | 3378 |
| | | SLL | | | | | | F | | | | YGYTF | | | 3865 |
| LD7 | Neo⁻WT⁺ | 0 | 0 | 0 | 0 | 0 | 1-1*01 | CAGRGYSTL | | | 27*01 | CASSSDSSY | | | 3379 |
| | | | | | | | | TF | | | | EQYF | | | 3866 |
| LD9 | Neo⁻WT⁺ | 0 | 0 | 0 | 0 | 0 | | | | | 9*01 | CASTPGGSS | | | 3867 |
| | | | | | | | | | | | | YNSPLHF | | | |
| LE11 | Neo⁻WT⁺ | SEC24A- | 0 | 0 | 0 | 0 | 12-2*01 | CAVTARSSY | | | | | | | 3380 |
| | | FLY | | | | | | KLIF | | | | | | | |
| LE5 | Neo⁻WT⁺ | BCL9L- | 0 | 0 | 0 | 0 | 12-2*01 | CAVGDSNYQ | | | 6-5*01 | CASSFNYNE | | | 3381 |
| | | FVY | | | | | | LIW | | | | OFF | | | 3868 |
| LF1 | Neo⁻WT⁺ | 0 | 0 | 0 | 0 | 0 | | | | | | | | | |
| LF10 | Neo⁻WT⁺ | TBX3- | 0 | 0 | 0 | 0 | 38-2/ | CAYRSFNNN | | | 13*01 | CASRSRGGH | | | 3382 |
| | | GMG | | | | | DV8*01 | DMRF | | | | SPLHF | | | 3869 |
| LF2 | Neo⁻WT⁺ | OR5M3- | 0 | 0 | 0 | 0 | | | | | | | | | |
| | | KMV | | | | | | | | | | | | | |
| LF3 | Neo⁻WT⁺ | 0 | 0 | 0 | 0 | 0 | | | | | | | | | |
| LF4 | Neo⁻WT⁺ | TBX3- | 0 | 0 | 0 | 0 | 17*01 | CATDNDMRF | | | 13*01 | CASSFGPDE | | | 3383 |
| | | GMG | | | | | | | | | | QYF | | | 3870 |
| LF5 | Neo⁻WT⁺ | 0 | 0 | 0 | 0 | 0 | 12-2*01 | CAPSLDMRF | | | | | | | 3384 |
| LF6 | Neo⁻WT⁺ | 0 | 0 | 0 | 0 | 0 | 4*01 | CLVGDGGVT | | | 28*01 | CASSSTGDN | | | 3385 |
| | | | | | | | | GGGNKLTF | | | | SPLHF | | | 3871 |
| LF8 | Neo⁻WT⁺ | 0 | 0 | 0 | 0 | 0 | 5*01 | CAESMERG | | | 28*01 | CASQSWRG | | | 3386 |
| | | | | | | | | DKLIF | | | | MNTEAFF | | | 3872 |
| LF9 | Neo⁻WT⁺ | OR5M3- | 0 | 0 | 0 | 0 | 1-1*01 | CAVVDSNYQ | | | 11-1*01 | CASSSPWG | | | 3387 |
| | | KMV | | | | | | LIW | | | | GTTDTSTDT | | | 3873 |
| | | | | | | | | | | | | QYF | | | |
| LG10 | Neo⁻WT⁺ | ITIH6- | 0 | 0 | 0 | 0 | 12-2*01 | CAVYGDYG | | | 11-2*01 | CASSRGGLT | | | 3388 |
| | | RLG | | | | | | GSQGNLIF | | | | DTQYF | | | 3874 |
| LG3 | Neo⁻WT⁺ | OR5M3- | 0 | 0 | 0 | 0 | | | | | | | | | |
| | | KMV | | | | | | | | | | | | | |
| LG5 | Neo⁻WT⁺ | GOLGA3- | 0 | 0 | 0 | 0 | | | | | | | | | |
| | | SLD | | | | | | | | | | | | | |
| LG6 | Neo⁻WT⁺ | KAT6A- | 0 | 0 | 0 | 0 | 19*01 | CALSEAEEY | | | 12-3*01, | CASSFLSSY | | | 3389 |
| | | KLS | | | | | | GNKLVF | | | 12-4*01 | NEQFF | | | 3875 |
| LG9 | Neo⁻WT⁺ | OR6F1- | 0 | 0 | 0 | 0 | | | | | | | | | |
| | | VLN | | | | | | | | | | | | | |
| LH11 | Neo⁻WT⁺ | 0 | 0 | 0 | 0 | 0 | | | | | | | | | |
| LH12 | Neo⁻WT⁺ | 0 | 0 | 0 | 0 | 0 | 12-2*01 | CAVKNTGRR | | | | | | | 3390 |
| | | | | | | | | ALTF | | | | | | | |
| MA11 | Neo⁻WT⁺ | 0 | 0 | 0 | 0 | 0 | 20*01 | CAVQAFGNE | | | 18*01 | CASSGPEAY | | | 3391 |
| | | | | | | | | KLTF | | | | EQYF | | | 3876 |
| MA12 | Neo⁻WT⁺ | SHROOM2- | 0 | 0 | 0 | 0 | 17*01 | CATGGVSNT | 25*01 | CAGYDYKL | 10-3*01 | CAISESKGN | | | 3392 |
| | | KLL | | | | | | NAGKSTF | | SF | | YGYTF | | | 3485 |
| | | | | | | | | | | | | | | | 3877 |
| MA6 | Neo⁻WT⁺ | OR5M3- | 0 | 0 | 0 | 0 | 9-2*01 | CALILTNFGN | | | 7-9*01 | CASSAPGQG | | | 3393 |
| | | KMV | | | | | | EKLTF | | | | NEKLFF | | | 3787 |
| MB11 | Neo⁻WT⁺ | 0 | 0 | 0 | 0 | 0 | | | | | | | | | |
| MB12 | Neo⁻WT⁺ | RYR3- | 0 | 0 | 0 | 0 | | | | | 19*01 | CASSIVDRPY | | | 3879 |
| | | VLN | | | | | | | | | | EQYF | | | |
| MB2 | Neo⁻WT⁺ | ITIH6- | 0 | 0 | 0 | 0 | 29/ | CAASVGDML | | | 15*01 | CATSRGTGA | | | 3394 |
| | | RLG | | | | | DV5*01 | TF | | | | GEQYF | | | 3880 |
| MB5 | Neo⁻WT⁺ | ITIH6- | 0 | 0 | 0 | 0 | 38-2/ | CAYTSNDMR | | | 7-4*01 | RASSPRTGG | 10-2*01 | CASSEFR | 3147 |
| | | RLG | | | | | DV8*01 | F | | | | EQYF | | NVGGYTF | 3881 |
| MB6 | Neo⁻WT⁺ | 0 | 0 | 0 | 0 | 0 | 3*01 | CAVRDNNFN | | | 6-6*01 | CASSYLDGA | | | 3395 |
| | | | | | | | | KFYF | | | | YEQYF | | | 3882 |
| MB7 | Neo⁻WT⁺ | MYPN- | MYPN- | 0 | 0 | 0 | 38-2/ | CAYMDSNY | | | 25-1*01 | CASSTGADL | | | 3396 |
| | | RVI_R1L | RVI | | | | DV8*01 | QLIW | | | | TYEQYF | | | 3883 |
| MC1 | Neo⁻WT⁺ | 0 | 0 | 0 | 0 | 0 | 38-2/ | CAYNQGGKL | | | 12-3*01, | CASSFTRDL | | | 3397 |
| | | | | | | | DV8*01 | IF | | | 12-4*01 | YGYTF | | | 3884 |
| MC11 | Neo⁻WT⁺ | OR5M3- | 0 | 0 | 0 | 0 | | | | | 7-9*01 | CASSLAVGE | 7-9*01 | CASLKMG | 3885 |
| | | KMV | | | | | | | | | | TRNSPLHF | | GLDEQFF | |
| MC2 | Neo⁻WT⁺ | 0 | 0 | 0 | 0 | 0 | | | | | 7-9*01 | CASSGTGGY | | | 3886 |
| | | | | | | | | | | | | EQYF | | | |
| MC3 | Neo⁻WT⁺ | OR5M3- | 0 | 0 | 0 | 0 | 4*01 | CLVGYSGGY | | | 7-9*01 | CASSLAGDR | | | 3398 |
| | | KMV | | | | | | QKVTF | | | | GRNSPLHF | | | 3887 |
| MC5 | Neo⁻WT⁺ | PIGN- | 0 | 0 | 0 | 0 | 12-2*01 | CAVVYSGGG | | | 19*01 | CASSPWTGA | | | 3399 |
| | | FLT | | | | | | ADGLTF | | | | EKLFF | | | 3888 |
| MC6 | Neo⁻WT⁺ | OR5M3- | 0 | 0 | 0 | 0 | | | | | 7-9*01 | CASSYFFEG | | | 3889 |
| | | KMV | | | | | | | | | | LNTGELFF | | | |
| MC9 | Neo⁻WT⁺ | LCP1- | 0 | 0 | 0 | 0 | | | | | 13*01 | CASSSPSGG | | | 3890 |
| | | NLF | | | | | | | | | | RTDTQYF | | | |
| MD10 | Neo⁻WT⁺ | OR5M3- | 0 | 0 | 0 | 0 | | | | | 7-9*01 | CASSFFASG | | | 3891 |
| | | KMV | | | | | | | | | | DTDTQYF | | | |
| MD2 | Neo⁻WT⁺ | VN1R2- | 0 | 0 | 0 | 0 | | | | | | | | | |
| | | LML | | | | | | | | | | | | | |
| MD6 | Neo⁻WT⁺ | 0 | 0 | 0 | 0 | 0 | 9-2*01 | CALTKETSG | | | 5-1*01 | CASSLEGTS | | | 3400 |
| | | | | | | | | SRLTF | | | | LNEQFF | | | 3892 |
| ME10 | Neo⁻WT⁺ | 0 | 0 | 0 | 0 | 0 | | | | | 2*01 | CASSPDSDH | | | 3893 |
| | | | | | | | | | | | | YGYTF | | | |
| ME5 | Neo⁻WT⁺ | 0 | 0 | 0 | 0 | 0 | | | | | 6-5*01 | CASSQFMNT | | | 3894 |
| | | | | | | | | | | | | EAFF | | | |
| ME6 | Neo⁻WT⁺ | 0 | 0 | 0 | 0 | 0 | 21*01 | CAVLNDYKL | 27*01 | CAGGTGY | 12-3*01, | CASSLQGNG | | | 3401 |
| | | | | | | | | SF | | NKLIF | 12-4*01 | YTF | | | 3486 |
| | | | | | | | | | | | | | | | 3895 |
| ME9 | Neo⁻WT⁺ | 0 | 0 | 0 | 0 | 0 | | | | | 5-5*01 | CASSLGGLS | | | 3896 |
| | | | | | | | | | | | | GYTF | | | |
| MF1 | Neo⁻WT⁺ | 0 | 0 | 0 | 0 | 0 | | | | | 27*01 | CASSFQGGT | | | 3897 |
| | | | | | | | | | | | | GYTF | | | |
| MF2 | Neo⁻WT⁺ | 0 | 0 | 0 | 0 | 0 | 4*01 | CLVGDPVDK | | | 6-1*01 | CASSEDGYE | | | 3402 |
| | | | | | | | | IIF | | | | QYF | | | 3898 |
| MF5 | Neo⁻WT⁺ | 0 | 0 | 0 | 0 | 0 | | | | | 6-2*01, | CASKNDGNS | | | 3899 |
| | | | | | | | | | | | 6-3*01 | PLHF | | | |
| MF6 | Neo⁻WT⁺ | SEC24A- | 0 | 0 | 0 | 0 | | | | | | | | | |
| | | FLY | | | | | | | | | | | | | |
| MG1 | Neo⁻WT⁺ | 0 | 0 | 0 | 0 | 0 | 17*01 | CATDEGGST | | | 13*01 | CASSLVTSG | | | 3403 |
| | | | | | | | | LGRLYF | | | | EQFF | | | 3900 |
| MG2 | Neo⁻WT⁺ | OR5M3- | 0 | 0 | 0 | 0 | | | | | | | | | |
| | | KMV | | | | | | | | | | | | | |
| MG4 | Neo⁻WT⁺ | OR5M3- | OR5M3- | 0 | 0 | 0 | 8-2*01 | CVVTISGGY | | | 11-2*01 | CASSLPDNN | | | 3404 |
| | | KMV | KMV_T8N | | | | | NKLIF | | | | EQFF | | | 3901 |
| MG7 | Neo⁻WT⁺ | 0 | 0 | 0 | 0 | 0 | 12-2*01 | CASGGGNM | | | 20-1*01 | CSATDVWGY | | | 3405 |
| | | | | | | | | LTF | | | | TF | | | 3902 |
| MG8 | Neo⁻WT⁺ | MRM1-9 | 0 | 0 | 0 | 0 | 12-2*01 | CAGNNARLM | | | 7-9*01 | CASSNLGGT | | | 3406 |
| | | | | | | | | F | | | | DTQYF | | | 3903 |
| MH11 | Neo⁻WT⁺ | KCNB2- | 0 | 0 | 0 | 0 | 19*01 | CALIYFSGGY | | | 7-6*01 | CASSSPSQG | | | 3407 |
| | | LLA | | | | | | NKLIF | | | | ITGELFF | | | 3904 |
| MH3 | Neo⁻WT⁺ | OR5M3- | 0 | 0 | 0 | 0 | 1-1*01 | CICEGGSYIP | | | 7-9*01 | CASSFWRD | | | 3408 |
| | | KMV | | | | | | TF | | | | GATNEKLFF | | | 3905 |
| MH5 | Neo⁻WT⁺ | HTR1F- | 0 | 0 | 0 | 0 | | | | | | | | | |
| | | 10 | | | | | | | | | | | | | |
| MH7 | Neo⁻WT⁺ | 0 | 0 | 0 | 0 | 0 | | | | | | | | | |
| NA10 | Neo⁻WT⁺ | HAUS3- | 0 | 0 | 0 | 0 | 21*01 | CAVITGGGN | | | | | | | 3409 |
| | | ILN | | | | | | KLTF | | | | | | | |
| NA4 | Neo⁻WT⁺ | SCN3A- | 0 | 0 | 0 | 0 | | | | | 27*01 | CASSFSARE | | | 3906 |
| | | ALV | | | | | | | | | | YGYTF | | | |
| NA5 | Neo⁻WT⁺ | 0 | 0 | 0 | 0 | 0 | 12-3*01 | CAMSGHDM | | | 27*01 | CASSFGANY | | | 3410 |
| | | | | | | | | RF | | | | GYTF | | | 3907 |
| NA7 | Neo⁻WT⁺ | LCP1- | 0 | 0 | 0 | 0 | 8-3*01 | CAVVRGDTD | | | 11-2*01 | CASSLYVYS | | | 3411 |
| | | NLF | | | | | | KLIF | | | | YEQYF | | | 3908 |
| NB2 | Neo⁻WT⁺ | LCP1- | 0 | 0 | 0 | 0 | 5*01 | CAEETGGGN | | | 11-2*01 | CASSLMGAE | | | 3412 |
| | | NLF | | | | | | KLTF | | | | AFF | | | 3909 |
| NC1 | Neo⁻WT⁺ | ATP6AP1- | KAT6A- | 0 | 0 | 0 | | | | | 4-1*01 | CASSQAGDG | | | 3910 |
| | | KLG | KLS | | | | | | | | | SYEQYF | | | |
| NC11 | Neo⁻WT⁺ | 0 | 0 | 0 | 0 | 0 | | | | | 3-1*01 | CASSQLDYN | | | 3911 |
| | | | | | | | | | | | | EQFF | | | |
| NC5 | Neo⁻WT⁺ | NOS1- | 0 | 0 | 0 | 0 | 38-1*01 | CAFIRGSQG | | | 11-2*01 | CASSFWSG | | | 3413 |
| | | FID | | | | | | NLIF | | | | GTYEQYF | | | 3912 |
| NC6 | Neo⁻WT⁺ | 0 | 0 | 0 | 0 | 0 | 26-2*01 | CILSYNTGN | 14/ | CAIIRFGN | 19*01 | CASSATSGA | | | 3414 |
| | | | | | | | | QFYF | DV4*01 | EKLTF | | YNEQFF | | | 3487 |
| | | | | | | | | | | | | | | | 3913 |
| NC9 | Neo⁻WT⁺ | 0 | 0 | 0 | 0 | 0 | | | | | 20-1*01 | CSARAVTNT | | | 3914 |
| | | | | | | | | | | | | GELFF | | | |
| ND1 | Neo⁻WT⁺ | 0 | 0 | 0 | 0 | 0 | | | | | | | | | |
| ND10 | Neo⁻WT⁺ | OR9Q2- | 0 | 0 | 0 | 0 | 12-2*01 | CAPRGSGR | | | 28*01 | CASSLQGGG | | | 3415 |
| | | FLF | | | | | | RALTF | | | | GYTF | | | 3915 |
| ND2 | Neo⁻WT⁺ | CD47- | APBB2- | 0 | 0 | 0 | | | | | | | | | |
| | | GLT | VQY_L7F | | | | | | | | | | | | |
| ND8 | Neo⁻WT⁺ | 0 | 0 | 0 | 0 | 0 | 35*01 | CAGPHLSYN | | | 14*01 | CASSQVGQ | | | 3416 |
| | | | | | | | | TDKLIF | | | | GQF | | | 3916 |
| ND9 | Neo⁻WT⁺ | ITIH6- | 0 | 0 | 0 | 0 | 8-3*01 | CAVGAGNN | | | 9*01 | CASSVYSTD | 4-3*01 | CASRVSA | 3417 |
| | | RLG | | | | | | DMRF | | | | TQYF | | SSYNEQF | 3917 |
| | | | | | | | | | | | | | | F | |
| NE10 | Neo⁻WT⁺ | 0 | 0 | 0 | 0 | 0 | | | | | 7-9*01 | CASSYLGRV | | | 3918 |
| | | | | | | | | | | | | NKNIQYF | | | |
| NE12 | Neo⁻WT⁺ | OR5M3- | 0 | 0 | 0 | 0 | 21*01 | CAVPSRPNF | 40*01 | CLLLNYGG | 7-9*01 | CASSLGGTE | | | 3418 |
| | | KMV | | | | | | GNEKLTF | | SQGNLIF | | AFF | | | 3488 |
| | | | | | | | | | | | | | | | 3919 |
| NE3 | Neo⁻WT⁺ | GABRG3- | 0 | 0 | 0 | 0 | | | | | 20-1*01 | CSARNRASY | | | 3920 |
| | | TAM | | | | | | | | | | NSPLHF | | | |
| NE7 | Neo⁻WT⁺ | 0 | 0 | 0 | 0 | 0 | 19*01 | CALPDIQGA | | | 18*01 | CASSQQGFY | | | 3419 |
| | | | | | | | | QKLVF | | | | EQYF | | | 3921 |
| NF1 | Neo⁻WT⁺ | 0 | 0 | 0 | 0 | 0 | 14/ | CAMREDYG | | | 15*01 | CATTPDRGH | | | 3420 |
| | | | | | | | DV4*01 | GSQGNLIF | | | | QPQHF | | | 3922 |
| NF10 | Neo⁻WT⁺ | OR5M3- | 0 | 0 | 0 | 0 | | | | | | | | | |
| | | KMV | | | | | | | | | | | | | |
| NF11 | Neo⁻WT⁺ | 0 | 0 | 0 | 0 | 0 | | | | | 6-5*01 | CASSYLEGD | | | 3923 |
| | | | | | | | | | | | | NYGYTF | | | |
| NF2 | Neo⁻WT⁺ | OR5M3- | 0 | 0 | 0 | 0 | | | | | | | | | |
| | | KMV | | | | | | | | | | | | | |
| NF5 | Neo⁻WT⁺ | OR5M3- | 0 | 0 | 0 | 0 | | | | | | | | | |
| | | KMV | | | | | | | | | | | | | |
| NG10 | Neo⁻WT⁺ | OR5M3- | 0 | 0 | 0 | 0 | 26-1*01 | CIVRVGYNA | | | 7-9*01 | CASSLGHFE | | | 3421 |
| | | KMV | | | | | | RLMF | | | | GNQPQHF | | | 3924 |
| NG12 | Neo⁻WT⁺ | 0 | 0 | 0 | 0 | 0 | | | | | | | | | |
| NG8 | Neo⁻WT⁺ | 0 | 0 | 0 | 0 | 0 | | | | | 29-1*01 | CSVTGNNYG | | | 3925 |
| | | | | | | | | | | | | YTF | | | |
| NH7 | Neo⁻WT⁺ | 0 | 0 | 0 | 0 | 0 | | | | | | | | | |
| NH8 | Neo⁻WT⁺ | ZDHHC7- | 0 | 0 | 0 | 0 | | | | | 7-9*01 | CASSSETNW | | | 3926 |
| | | SLL | | | | | | | | | | GTGGNQPQ | | | |
| | | | | | | | | | | | | HF | | | |
| OA10 | Neo⁻WT⁺ | NOS1- | 0 | 0 | 0 | 0 | 34*01 | CGAVFLNDY | | | 27*01 | CASSMTVMN | | | 3422 |
| | | FID | | | | | | KLSF | | | | TEAFF | | | 3927 |
| OA11 | Neo⁻WT⁺ | DHX33- | OR1G1- | 0 | 0 | 0 | 22*01 | CAVDIATFG | | | 9*01 | CASSVDFGR | | | 3423 |
| | | LLA_K5T | FLF | | | | | NEKLTF | | | | TYNEQFF | | | 3928 |
| OA12 | Neo⁻WT⁺ | OR5M3- | 0 | 0 | 0 | 0 | | | | | | | | | |
| | | KMV | | | | | | | | | | | | | |
| OA2 | Neo⁻WT⁺ | OR5M3- | DHX33- | 0 | 0 | 0 | 25*01F | STSFGSNYG | 8-6*01 | CAVSVGVK | 7-9*01 | CASSLVPSG | | | 3424 |
| | | KMV | LLA_K5T | | | | | QNFVF | | YNFNKFYF | | QANTEAFF | | | 3489 |
| | | | | | | | | | | | | | | | 3929 |
| OA3 | Neo⁻WT⁺ | 0 | 0 | 0 | 0 | 0 | 10*01 | CVVLGGYNK | | | | | | | 3425 |
| | | | | | | | | LIF | | | | | | | |
| OA4 | Neo⁻WT⁺ | 0 | 0 | 0 | 0 | 0 | | | | | | | | | |
| OA7 | Neo⁻WT⁺ | HCV- | 0 | 0 | 0 | 0 | | | | | 6-2*01, | CASSYRGVE | | | 3930 |
| | | KLV(APC) | | | | | | | | | 6-3*01 | QYF | | | |
| OA9 | Neo⁻WT⁺ | 0 | 0 | 0 | 0 | 0 | 19*01 | CALSEAGDY | | | 3-1*01 | CASSTEGRS | | | 3426 |
| | | | | | | | | KLSF | | | | SYEQYF | | | 3931 |
| OB1 | Neo⁻WT⁺ | 0 | 0 | 0 | 0 | 0 | 22*01 | CAVYDNFNK | | | | | | | 3427 |
| | | | | | | | | FYF | | | | | | | |
| OB3 | Neo⁻WT⁺ | 0 | 0 | 0 | 0 | 0 | | | | | | | | | |
| OB5 | Neo⁻WT⁺ | NSDHL- | NSDHL- | 0 | 0 | 0 | 19*01 | CALMMTTDS | | | | | | | 3428 |
| | | ILT_A9V | ILT | | | | | WGKLQF | | | | | | | |
| OB8 | Neo⁻WT⁺ | 0 | 0 | 0 | 0 | 0 | | | | | | | | | |
| OC1 | Neo⁻WT⁺ | OR5M3- | 0 | 0 | 0 | 0 | 38-2/ | CACTGGGA | | | 27*01 | CASSLSPTD | | | 3429 |
| | | KMV | | | | | DV8*01 | DGLTF | | | | TQYF | | | 3932 |
| OC11 | Neo⁻WT⁺ | KCNB2- | 0 | 0 | 0 | 0 | 19*01 | CALNTIRDSN | | | 20-1*01 | CSARVRGDH | | | 3430 |
| | | LLA | | | | | | YQLIW | | | | NEQFF | | | 3933 |
| OC5 | Neo⁻WT⁺ | 0 | 0 | 0 | 0 | 0 | | | | | 7-9*01 | CASSSYTDK | | | 3934 |
| | | | | | | | | | | | | KSPGELFF | | | |
| OC6 | Neo⁻WT⁺ | 0 | 0 | 0 | 0 | 0 | | | | | 7-9*01 | CASSPTDTQ | | | 3935 |
| | | | | | | | | | | | | YF | | | |
| OC7 | Neo⁻WT⁺ | OR5M3- | 0 | 0 | 0 | 0 | | | | | 7-9*01 | CASSLERGM | | | 3936 |
| | | KMV | | | | | | | | | | GSNQPQHF | | | |
| OC8 | Neo⁻WT⁺ | 0 | 0 | 0 | 0 | 0 | | | | | 7-9*01 | CASSDWTGS | | | 3937 |
| | | | | | | | | | | | | NEQFF | | | |
| OC9 | Neo⁻WT⁺ | 0 | 0 | 0 | 0 | 0 | | | | | | | | | |
| OD1 | Neo⁻WT⁺ | 0 | 0 | 0 | 0 | 0 | | | | | 6-5*01 | CASSNTGGR | | | 3938 |
| | | | | | | | | | | | | ETQYF | | | |
| OD12 | Neo⁻WT⁺ | 0 | 0 | 0 | 0 | 0 | | | | | | | | | |
| OD9 | Neo⁻WT⁺ | 0 | 0 | 0 | 0 | 0 | 8-4*01 | CAVSEYDKII | | | 12-3*01, | CASSSSGGG | | | 3431 |
| | | | | | | | | F | | | 12-4*01 | TEQFF | | | 3939 |
| OE12 | Neo⁻WT⁺ | 0 | 0 | 0 | 0 | 0 | | | | | 10-3*01 | CATWTGGG | | | 3940 |
| | | | | | | | | | | | | SEAFF | | | |
| OE4 | Neo⁻WT⁺ | 0 | 0 | 0 | 0 | 0 | 5*01 | CAEIISSASK | | | | | | | 3432 |
| | | | | | | | | IIF | | | | | | | |
| OE9 | Neo⁻WT⁺ | PELP1- | PELP1- | 0 | 0 | 0 | 19*01 | CARLTGANN | | | | | | | 3433 |
| | | LVL | LVL_L3F | | | | | LFF | | | | | | | |
| OF11 | Neo⁻WT⁺ | ITIH6- | 0 | 0 | 0 | 0 | | | | | | | | | |
| | | RLG | | | | | | | | | | | | | |
| OF12 | Neo⁻WT⁺ | 0 | 0 | 0 | 0 | 0 | | | | | | | | | |
| OF4 | Neo⁻WT⁺ | 0 | 0 | 0 | 0 | 0 | | | | | | | | | |
| OF5 | Neo⁻WT⁺ | OR5M3- | 0 | 0 | 0 | 0 | | | | | | | | | |
| | | KMV | | | | | | | | | | | | | |
| OG1 | Neo⁻WT⁺ | HTR1F- | 0 | 0 | 0 | 0 | | | | | | | | | |
| | | 10 | | | | | | | | | | | | | |
| OG10 | Neo⁻WT⁺ | 0 | 0 | 0 | 0 | 0 | 12-2*01 | CAVSPFSDG | | | | | | | 3434 |
| | | | | | | | | QKLLF | | | | | | | |
| OG11 | Neo⁻WT⁺ | OR5M3- | 0 | 0 | 0 | 0 | | | | | | | | | |
| | | KMV | | | | | | | | | | | | | |
| OG4 | Neo⁻WT⁺ | 0 | 0 | 0 | 0 | 0 | | | | | | | | | |
| OG6 | Neo⁻WT⁺ | OR5M3- | OR5M3- | 0 | 0 | 0 | 26-2*01 | CILRDMEYG | | | 7-9*01 | CASSRYGGP | | | 3435 |
| | | KMV | KMV_T8N | | | | | NKLVF | | | | SDNEQFF | | | 3941 |
| OG8 | Neo⁻WT⁺ | ITIH6- | 0 | 0 | 0 | 0 | 38-2/ | CAFNDYKLS | | | 10-3*01 | CAIRDRLNTE | | | 3436 |
| | | RLG | | | | | DV8*01 | F | | | | AFF | | | 3942 |
| OH1 | Neo⁻WT⁺ | 0 | 0 | 0 | 0 | 0 | | | | | | | | | |
| OH12 | Neo⁻WT⁺ | HTR1F- | 0 | 0 | 0 | 0 | 10*01 | CVVSGGYNK | | | 4-2*01 | CASSQGTSR | | | 3328 |
| | | 10 | | | | | | LIF | | | | DRNQPQHF | | | 3943 |
| OH5 | Neo⁻WT⁺ | 0 | 0 | 0 | 0 | 0 | 13-1*01 | CAASRLPGY | | | 7-9*01 | CASTLGGEG | | | 3437 |
| | | | | | | | | SSASKIIF | | | | RNTGELFF | | | 3944 |
| OH7 | Neo⁻WT⁺ | ST6GALNAC2- | 0 | 0 | 0 | 0 | 12-3*01 | CAMKDNDM | | | 5-4*01 | CARGSGGET | | | 3438 |
| | | LLF | | | | | | RF | | | | QYF | | | 3945 |
| SA12 | Neo⁻WT⁺ | 0 | 0 | 0 | 0 | 0 | | | | | | | | | |
| SA3 | Neo⁻WT⁺ | ERBB2- | 0 | 0 | 0 | 0 | 12-2*01 | CAVNSNSGY | | | 4-1*01 | CASSQSETG | | | 3439 |
| | | ALI | | | | | | ALNF | | | | DGYTF | | | 3946 |
| SB10 | Neo⁻WT⁺ | 0 | 0 | 0 | 0 | 0 | | | | | | | | | |
| SB11 | Neo⁻WT⁺ | 0 | 0 | 0 | 0 | 0 | | | | | | | | | |
| SB12 | Neo⁻WT⁺ | 0 | 0 | 0 | 0 | 0 | | | | | | | | | |
| SB3 | Neo⁻WT⁺ | KCNB2- | 0 | 0 | 0 | 0 | | | | | 19*01 | CASSITFSDT | | | 3947 |
| | | LLA | | | | | | | | | | QYF | | | |
| SB5 | Neo⁻WT⁺ | ZNF827- | 0 | 0 | 0 | 0 | 17*01 | CASSGGSYI | | | | | | | 3440 |
| | | NLF | | | | | | PTF | | | | | | | |
| SB6 | Neo⁻WT⁺ | 0 | 0 | 0 | 0 | 0 | 12-2*01 | CAVNDYKLS | | | 4-1*01 | CASSQALDQ | | | 3441 |
| | | | | | | | | F | | | | PQHF | | | 3948 |
| SB7 | Neo⁻WT⁺ | SCN3A- | 0 | 0 | 0 | 0 | 14/ | CAMREHGTA | | | | | | | 3442 |
| | | ALV | | | | | DV4*01 | GNKLTF | | | | | | | |
| SB9 | Neo⁻WT⁺ | 0 | 0 | 0 | 0 | 0 | | | | | | | | | |
| SC12 | Neo⁻WT⁺ | 0 | 0 | 0 | 0 | 0 | | | | | 19*01 | CASSNRDRG | | | 3949 |
| | | | | | | | | | | | | PYEQYF | | | |
| SC4 | Neo⁻WT⁺ | 0 | 0 | 0 | 0 | 0 | | | | | | | | | |
| SC5 | Neo⁻WT⁺ | ME1- | 0 | 0 | 0 | 0 | 14/ | CAMRERTG | | | 20-1*01 | CSARQTSGG | | | 3443 |
| | | FLD | | | | | DV4*01 | GFKTIF | | | | SSYNEQFF | | | 3950 |
| SC7 | Neo⁻WT⁺ | ITIH6- | 0 | 0 | 0 | 0 | | | | | | | | | |
| | | RLG | | | | | | | | | | | | | |
| SC8 | Neo⁻WT⁺ | 0 | 0 | 0 | 0 | 0 | | | | | | | | | |
| SD7 | Neo⁻WT⁺ | 0 | 0 | 0 | 0 | 0 | 12-2*01 | CAVMTTDS | | | 7-9*01 | CASSSLGLF | | | 3444 |
| | | | | | | | | WGKLQF | | | | AEQFF | | | 3951 |
| SD8 | Neo⁻WT⁺ | NSDHL- | NSDHL- | 0 | 0 | 0 | 14/ | CAMRETPQ | | | 2*01 | CASSEGQNT | | | 3445 |
| | | ILT | ILT_A9V | | | | DV4*01 | GGSEKLVF | | | | EAFF | | | 3952 |
| SE11 | Neo⁻WT⁺ | 0 | 0 | 0 | 0 | 0 | 24*01 | CAFINDYKLS | | | 6-2*01, | CASSTGPYN | | | 3446 |
| | | | | | | | | F | | | 6-3*01 | EQFF | | | 3953 |
| SE3 | Neo⁻WT⁺ | GPR174- | 0 | 0 | 0 | 0 | 20*01 | CAVSDTGGF | | | 7-8*01 | CASSLTGSS | | | 3447 |
| | | FSF | | | | | | KTIF | | | | DTQYF | | | 3954 |
| SE5 | Neo⁻WT⁺ | 0 | 0 | 0 | 0 | 0 | | | | | | | | | |
| SE6 | Neo⁻WT⁺ | OR5M3- | 0 | 0 | 0 | 0 | | | | | | | | | |
| | | KMV | | | | | | | | | | | | | |
| SE8 | Neo⁻WT⁺ | 0 | 0 | 0 | 0 | 0 | 12-1*01 | CVVNMEGG | | | 14*01 | CASSQAGQ | | | 3448 |
| | | | | | | | | GADGLTF | | | | GFRTEAFF | | | 3995 |
| SE9 | Neo⁻WT⁺ | 0 | 0 | 0 | 0 | 0 | | | | | | | | | |
| SF10 | Neo⁻WT⁺ | 0 | 0 | 0 | 0 | 0 | | | | | | | | | |
| SF3 | Neo⁻WT⁺ | HTR1F- | 0 | 0 | 0 | 0 | | | | | | | | | |
| | | 10 | | | | | | | | | | | | | |
| SF6 | Neo⁻WT⁺ | 0 | 0 | 0 | 0 | 0 | | | | | | | | | |
| SF8 | Neo⁻WT⁺ | OR5M3- | 0 | 0 | 0 | 0 | | | | | 7-9*01 | CASSLGQER | | | 3956 |
| | | KMV | | | | | | | | | | PYEQYF | | | |
| SG10 | Neo⁻WT⁺ | 0 | 0 | 0 | 0 | 0 | | | | | | | | | |
| SG11 | Neo⁻WT⁺ | 0 | 0 | 0 | 0 | 0 | | | | | | | | | |
| SG12 | Neo⁻WT⁺ | 0 | 0 | 0 | 0 | 0 | | | | | | | | | |
| SG3 | Neo⁻WT⁺ | 0 | 0 | 0 | 0 | 0 | | | | | | | | | |
| SG5 | Neo⁻WT⁺ | HTR1F- | 0 | 0 | 0 | 0 | | | | | | | | | |
| | | 10 | | | | | | | | | | | | | |
| SG6 | Neo⁻WT⁺ | GLRA1- | 0 | 0 | 0 | 0 | | | | | 5-1*01 | CASSFGQGY | | | 3957 |
| | | LIF | | | | | | | | | | EQYF | | | |
| SG9 | Neo⁻WT⁺ | 0 | 0 | 0 | 0 | 0 | | | | | | | | | |
| SH10 | Neo⁻WT⁺ | 0 | 0 | 0 | 0 | 0 | | | | | | | | | |
| SH11 | Neo⁻WT⁺ | 0 | 0 | 0 | 0 | 0 | | | | | | | | | |
| SH3 | Neo⁻WT⁺ | 0 | 0 | 0 | 0 | 0 | | | | | | | | | |
| SH5 | Neo⁻WT⁺ | ITIH6- | 0 | 0 | 0 | 0 | 19*01 | CALSEDQFY | | | 6-1*01 | CASRPGGGS | | | 3449 |
| | | RLG | | | | | | F | | | | YNEQFF | | | 3958 |
| SH7 | Neo⁻WT⁺ | ITIH6- | 0 | 0 | 0 | 0 | | | | | | | | | |
| | | RLG | | | | | | | | | | | | | |

TABLE 10
Experiment 1

| | | Tetramer | |
|---|---|---|---|
| Peptide Name | Sequence | Fluorescence | SEQ ID NO: |
| NYESO1-V165 | SLLMWITQV | PE | 3964 |
| ADI-SVA | SVASTITGV | PE | 3965 |
| BRA-AG | WLLPGTSTV | PE | 3966 |
| BRA-NA | WLLPGTSTL | PE | 3967 |
| CD1-LLG | LLGATCMFV | PE | 3968 |
| GP100-IMD | IMDQVPFSV | PE | 3969 |
| GP100-AML | AMLGTHTMEV | PE | 3970 |
| GP100-ITD | ITDQVPFSV | PE | 3971 |
| GP100-KTW | KTWGQYWQV | PE | 3972 |
| GP100-YLE | YLEPGPVTA | PE | 3973 |
| GPC-FVG | FVGEFFTDV | PE | 3974 |
| HAFP-FMN | FMNKFIYEI | PE | 3975 |
| HAFP-GLS | GLSPNLNRFL | PE | 3976 |
| MAGEA10-GLY | GLYDGMEHL | PE | 3977 |
| MAGEC2-LLF | LLFGLALIEV | PE | 3978 |
| MART1-A2L | ELAGIGILTV | PE | 3979 |
| MART1-ALM | ALMDKSLHV | PE | 3980 |
| MG50-CMH | CMHLLLEAV | PE | 3981 |
| NYESO1-9A | SLLMWITQA | PE | 3982 |
| TYR-YMD | YMDGTMSQV | PE | 3983 |
| TYR-CLL | CLLWSFQTSA | PE | 3984 |
| WT1-RMF | RMFPNAPYL | PE | 3985 |
| AGL-GLI | QLIPCMDVV | PE | 3986 |
| EF2-ILT | ILTDITKGV | PE | 3987 |
| FBA-ALS | ALSDHHIYL | PE | 3988 |
| HA-VLH | VLHDDLLEA | PE | 3989 |
| KER-ALL | ALLNIKVKL | PE | 3990 |
| L19-ILM | ILMEHIHKL | PE | 3991 |
| PD5-KLS | KLSEGDLLA | PE | 3992 |
| PP1-SII | SIIGRLLEV | PE | 3993 |
| DDX5-YLL | YLLPAIVHI | PE | 3994 |
| SMCY-FID | FIDSYICQV | PE | 3995 |
| SNPG-IML | IMLEALERV | PE | 3996 |
| GAD-RMM | RMMEYGTTMV | PE | 3997 |
| GAD65-VMN | VMNILLQYVV | PE | 3998 |
| GFAP-NLA | NLAQTDLATV | PE | 3999 |
| HCHGA-LLC | LLCAGQVTAL | PE | 4000 |
| HCHGA-TLS | TLSKPSPMPV | PE | 4001 |
| IA2-MVW | MVWESGCTV | PE | 4002 |
| IA2-VIV | VIVMLTPLV | PE | 4003 |
| IA2-SLY | SLYHVYEVNL | PE | 4004 |
| IA2-SLS | SLSPLQAEL | PE | 4005 |
| IA2-SLA | SLAAGVKLL | PE | 4006 |
| IAPP-KLQ | KLQVFLIVL | PE | 4007 |
| IAPP-FLI | FLIVLSVAL | PE | 4008 |
| IGRP-VLF | VLFGLGFAI | PE | 4009 |
| IGRP-RLL | RLLCALTSL | PE | 4010 |
| IGRP-FLW | FLWSVFMLI | PE | 4011 |
| IGRP-FLF | FLFAVGFYL | PE | 4012 |
| INS-HLV | HLVEALYLV | PE | 4013 |
| INS-SHL | SHLVEALYLV | PE | 4014 |
| DRIP-MLY | MLYQHLLPL | PE | 4015 |
| PPI-15-23 | ALWGPDPAA | PE | 4016 |
| PPI-15-24 | ALWGPDPAAA | PE | 4017 |
| PPI-RLL | RLLPLLALL | PE | 4018 |
| PPI-ALVVM | ALWMRLLPL | PE | 4019 |
| ZNT8-VAA | VAANIVLTV | PE | 4020 |
| ZNT8-LLI | LLIDLTSFLL | PE | 4021 |
| ZNT8-LLS | LLSLFSLWL | PE | 4022 |
| ZNT8-WT | VVTGVLVYL | PE | 4023 |
| ZNT8-VMI | VMIIVSSLAV | PE | 4024 |
| ZNT8-ILA | ILAVDGVLSV | PE | 4025 |
| HCV-K1S | SLVALGINAV | APC | 4026 |
| HCV-K1Y | YLVALGINAV | APC | 4027 |
| HCV-K1Y17V | YLVALGVNAV | APC | 4028 |
| HCV-L2I | KIVALGINAV | APC | 4029 |
| HCV-KLV (WT) | KLVALGINAV | APC | 4030 |
| CMV-VLE | VLEETSVML | APC | 4031 |
| CMV-MLN | MLNIPSINV | APC | 4032 |
| CMV-NLV | NLVPMVATV | APC | 4033 |
| EBV-GLC | GLCTLVAML | APC | 4034 |
| EBV-YVL | YVLDHLIVV | APC | 4035 |
| EBV-YLQ | YLQQNWWTL | APC | 4036 |
| EBV-CLG | CLGGLLTMV | APC | 4037 |
| EBV-FLY | FLYALALLL | APC | 4038 |
| HCV-FLP | FLPSDFFPSV | APC | 4039 |
| HBV-WLS | WLSLLVPFV | APC | 4040 |
| HCV-YLL | YLLPRRGPRL | APC | 4041 |
| HCV-CIN | CINGVCWTV | APC | 4042 |
| HCV-LLF | LLFNILGGWV | APC | 4043 |
| HIV-ILK | ILKEPVHGV | APC | 4044 |
| HIV-SLY | SLYNTVATL | APC | 4045 |
| HPV-YML | YMLDLQPETT | APC | 4046 |
| HSV-SLP | SLPITVYYA | APC | 4047 |
| HTLV-GLL | GLLSLEEEL | APC | 4048 |
| HTLV-LLF | LLFGYPVYV | APC | 4049 |
| IV-AIM | AIMDKNIIL | APC | 4050 |
| IV-GIL | GILGFVFTL | APC | 4051 |
| IVPA-FMY | FMYSDFHFI | APC | 4052 |
| MEA-SMY | SMYRVFEVGV | APC | 4053 |
| MEA-ILP | ILPGQDLQYV | APC | 4054 |
| YFV-LLW | LLWNGPMAV | APC | 4055 |
| ALADH-VLM | VLMGGVPGVE | APC | 4056 |
| GLNS-GLL | GLLHHAPSL | APC | 4057 |
| SODA-DMW | DMWEHAFYL | APC | 4058 |
| Empty | EMPTY | APC | 4059 |

Experiment 2

| | | Tetramer | |
|---|---|---|---|
| Peptide Name | Sequence | Fluorescence | SEQ ID NO: |
| NYESO1-V165 | SLLMWITQV | PE | 4060 |
| ADI-SVA | SVASTITGV | PE | 4061 |
| BRA-AG | WLLPGTSTV | PE | 4062 |
| BRA-NA | WLLPGTSTL | PE | 4063 |
| CD1-LLG | LLGATCMFV | PE | 4064 |
| GP100-IMD | IMDQVPFSV | PE | 4065 |
| GP100-AML | AMLGTHTMEV | PE | 4066 |
| GP100-ITD | ITDQVPFSV | PE | 4067 |
| GP100-KTW | KTWGQYWQV | PE | 4068 |
| GP100-YLE | YLEPGPVTA | PE | 4069 |
| GPC-FVG | FVGEFFTDV | PE | 4070 |
| HAFP-FMN | FMNKFIYEI | PE | 4071 |
| HAFP-GLS | GLSPNLNRFL | PE | 4072 |
| MAGEA10-GLY | GLYDGMEHL | PE | 4073 |
| MAGEC2-LLF | LLFGLALIEV | PE | 4074 |
| MART1-A2L | ELAGIGILTV | PE | 4075 |
| MART1-ALM | ALMDKSLHV | PE | 4076 |
| MG50-CMH | CMHLLLEAV | PE | 4077 |
| NYESO1-9A | SLLMWITQA | PE | 4078 |
| TYR-YMD | YMDGTMSQV | PE | 4079 |
| TYR-CLL | CLLWSFQTSA | PE | 4080 |
| WT1-RMF | RMFPNAPYL | PE | 4081 |
| AGL-GLI | QLIPCMDVV | PE | 4082 |
| EF2-ILT | ILTDITKGV | PE | 4083 |
| FBA-ALS | ALSDHHIYL | PE | 4084 |
| HA-VLH | VLHDDLLEA | PE | 4085 |
| KER-ALL | ALLNIKVKL | PE | 4086 |
| L19-ILM | ILMEHIHKL | PE | 4087 |
| PD5-KLS | KLSEGDLLA | PE | 4088 |
| PP1-SII | SIIGRLLEV | PE | 4089 |
| DDX5-YLL | YLLPAIVHI | PE | 4090 |
| SMCY-FID | FIDSYICQV | PE | 4091 |
| SNPG-IML | IMLEALERV | PE | 4092 |
| GAD-RMM | RMMEYGTTMV | PE | 4093 |
| GAD65-VMN | VMNILLQYVV | PE | 4094 |
| GFAP-NLA | NLAQTDLATV | PE | 4095 |
| HCHGA-LLC | LLCAGQVTAL | PE | 4096 |
| HCHGA-TLS | TLSKPSPMPV | PE | 4097 |
| IA2-MVW | MVWESGCTV | PE | 4098 |
| IA2-VIV | VIVMLTPLV | PE | 4099 |
| IA2-SLY | SLYHVYEVNL | PE | 4100 |
| IA2-SLS | SLSPLQAEL | PE | 4101 |
| IA2-SLA | SLAAGVKLL | PE | 4102 |
| IAPP-KLQ | KLQVFLIVL | PE | 4103 |
| IAPP-FLI | FLIVLSVAL | PE | 4104 |
| IGRP-VLF | VLFGLGFAI | PE | 4105 |
| IGRP-RLL | RLLCALTSL | PE | 4106 |
| IGRP-FLW | FLWSVFMLI | PE | 4107 |
| IGRP-FLF | FLFAVGFYL | PE | 4108 |
| INS-HLV | HLVEALYLV | PE | 4109 |
| INS-SHL | SHLVEALYLV | PE | 4110 |
| DRIP-MLY | MLYQHLLPL | PE | 4111 |
| PPI-15-23 | ALWGPDPAA | PE | 4112 |
| PPI-15-24 | ALWGPDPAAA | PE | 4113 |
| PPI-RLL | RLLPLLALL | PE | 4114 |
| PPI-ALVVM | ALVVMRLLPL | PE | 4115 |
| ZNT8-VAA | VAANIVLTV | PE | 4116 |
| ZNT8-LLI | LLIDLTSFLL | PE | 4117 |
| ZNT8-LLS | LLSLFSLWL | PE | 4118 |
| ZNT8-VVT | VVTGVLVYL | PE | 4119 |
| ZNT8-VMI | VMIIVSSLAV | PE | 4120 |
| ZNT8-ILA | ILAVDGVLSV | PE | 4121 |
| HCV-K1S | SLVALGINAV | APC | 4122 |
| HCV-K1Y | YLVALGINAV | APC | 4123 |
| HCV-K1Y17V | YLVALGVNAV | APC | 4124 |
| HCV-L21 | KIVALGINAV | APC | 4125 |
| HCV-KLV (WT) | KLVALGINAV | APC | 4126 |
| CMV-VLE | VLEETSVML | APC | 4127 |
| CMV-MLN | MLNIPSINV | APC | 4128 |
| CMV-NLV | NLVPMVATV | APC | 4129 |
| EBV-GLC | GLCTLVAML | APC | 4130 |
| EBV-YVL | YVLDHLIVV | APC | 4131 |
| EBV-YLQ | YLQQNWWTL | APC | 4132 |
| EBV-CLG | CLGGLLTMV | APC | 4133 |
| EBV-FLY | FLYALALLL | APC | 4134 |
| HCV-FLP | FLPSDFFPSV | APC | 4135 |
| HBV-WLS | WLSLLVPFV | APC | 4136 |
| HCV-YLL | YLLPRRGPRL | APC | 4137 |
| HCV-CIN | CINGVCVVTV | APC | 4138 |
| HCV-LLF | LLFNILGGWV | APC | 4139 |
| HIV-ILK | ILKEPVHGV | APC | 4140 |
| HIV-SLY | SLYNTVATL | APC | 4141 |
| HPV-YML | YMLDLQPETT | APC | 4142 |
| HSV-SLP | SLPITVYYA | APC | 4143 |
| HTLV-GLL | GLLSLEEEL | APC | 4144 |
| HTLV-LLF | LLFGYPVYV | APC | 4145 |
| IV-AIM | AIMDKNIIL | APC | 4146 |
| IV-GIL | GILGFVFTL | APC | 4147 |
| IVPA-FMY | FMYSDFHFI | APC | 4148 |
| MEA-SMY | SMYRVFEVGV | APC | 4149 |
| MEA-ILP | ILPGQDLQYV | APC | 4150 |
| YFV-LLW | LLWNGPMAV | APC | 4151 |
| ALADH-VLM | VLMGGVPGVE | APC | 4152 |
| GLNS-GLL | GLLHHAPSL | APC | 4153 |
| SODA-DMW | DMWEHAFYL | APC | 4154 |
| HCV-A9N | KLVALGINNV | APC | 4155 |

Experiment 3

| | | Tetramer | |
|---|---|---|---|
| Peptide Name | Sequence | Fluorescence | SEQ ID NO |
| WDR46 | FLTYLDVSV | PE | 4156 |
| AHNAK | SMPDFDLHL | PE | 4157 |
| COL18A1 | VLLGVKLSGV | PE | 4158 |
| ERBB2 | ALIHHNTHL | PE | 4159 |
| TEAD1 (VLE) | VLENFTILLV | PE | 4160 |
| TEAD1 (SVL) | SVLENFTILL | PE | 4161 |
| NSDHL | ILTGLNYEA | PE | 4162 |
| GANAB | ALYGSVPVL | PE | 4163 |
| FNDC3B | VVLSWAPPV | PE | 4164 |
| GCN1L1 | ALLETLSLLL | PE | 4165 |
| MLL2 | ALSPVIPLI | PE | 4166 |
| SMARCD3 | KLFEFLVHGV | PE | 4167 |
| GNL3L | NLNRCSVPV | PE | 4168 |
| USP28 | LIIPCIHLI | PE | 4169 |
| MRM1 | LLFGMTPCL | PE | 4170 |
| SNX24 | KLSHQPVLL | PE | 4171 |
| PGM5 | AVGSHVYSV | PE | 4172 |
| SEC24A | FLYNPLTRV | PE | 4173 |
| AKAP13 | KLMNIQQQL | PE | 4174 |
| PABPC1 | MLGERLFPL | PE | 4175 |
| WDR46 T3I | FLIYLDVSV | APC | 4176 |
| AHNAK S1F | FMPDFDLHL | APC | 4177 |
| COL18A1 S8F | VLLGVKLFGV | APC | 4178 |
| ERBB2 H8Y | ALIHHNTYL | APC | 4179 |
| TEAD1 L8F | VLENFTIFLV | APC | 4180 |
| TEAD1 L9F | SVLENFTIFL | APC | 4181 |
| NSDHL A9V | ILTGLNYEV | APC | 4182 |
| GANAB S5F | ALYGFVPVL | APC | 4183 |
| FNDC3B L3M | VVMSWAPPV | APC | 4184 |
| GCN1L1 L6P | ALLETPSLLL | APC | 4185 |
| MLL2 L8H | ALSPVIPHI | APC | 4186 |
| SMARCD3 H8Y | KLFEFLVYGV | APC | 4187 |
| GNL3L R4C | NLNCCSVPV | APC | 4188 |
| USP28 C5F | LIIPFIHLI | APC | 4189 |
| MRM1 T6P | LLFGMPPCL | APC | 4190 |
| SNX24 P6L | KLSHQLVLL | APC | 4191 |
| PGM5 H5Y | AVGSYVYSV | APC | 4192 |
| SEC24A P5L | FLYNLLTRV | APC | 4193 |
| AKAP13 Q8K | KLMNIQQKL | APC | 4194 |
| PABPC1 R5Q | MLGEQLFPL | APC | 4195 |
| HCV-KLV (WT) | KLVALGINAV | PE | 4196 |
| HCV-KLV (WT) | KLVALGINAV | APC | 4197 |
| EMPTY | | APC | 4198 |
| EMPTY | | PE | 4199 |

Experiment 4

| | | Tetramer | |
|---|---|---|---|
| Peptide Name | Sequence | Fluorescence | SEQ ID NO |
| WDR46 | FLTYLDVSV | PE | 4200 |
| AHNAK | SMPDFDLHL | PE | 4201 |
| COL18A1 | VLLGVKLSGV | PE | 4202 |
| ERBB2 | ALIHHNTHL | PE | 4203 |
| TEAD1 (VLE) | VLENFTILLV | PE | 4204 |
| TEAD1 (SVL) | SVLENFTILL | PE | 4205 |
| NSDHL | ILTGLNYEA | PE | 4206 |
| GANAB | ALYGSVPVL | PE | 4207 |
| FNDC3B | VVLSWAPPV | PE | 4208 |
| GCN1L1 | ALLETLSLLL | PE | 4209 |
| MLL2 | ALSPVIPLI | PE | 4210 |
| SMARCD3 | KLFEFLVHGV | PE | 4211 |
| GNL3L | NLNRCSVPV | PE | 4212 |
| USP28 | LIIPCIHLI | PE | 4213 |
| MRM1 | LLFGMTPCL | PE | 4214 |
| SNX24 | KLSHQPVLL | PE | 4215 |
| PGM5 | AVGSHVYSV | PE | 4216 |
| SEC24A | FLYNPLTRV | PE | 4217 |
| AKAP13 | KLMNIQQQL | PE | 4218 |
| PABPC1 | MLGERLFPL | PE | 4219 |
| WDR46 T3I | FLIYLDVSV | APC | 4220 |
| AHNAK S1F | FMPDFDLHL | APC | 4221 |
| COL18A1 S8F | VLLGVKLFGV | APC | 4222 |
| ERBB2 H8Y | ALIHHNTYL | APC | 4223 |
| TEAD1 L8F | VLENFTIFLV | APC | 4224 |
| TEAD1 L9F | SVLENFTIFL | APC | 4225 |
| NSDHL A9V | ILTGLNYEV | APC | 4226 |
| GANAB S5F | ALYGFVPVL | APC | 4227 |
| FNDC3B L3M | VVMSWAPPV | APC | 4228 |
| GCN1L1 L6P | ALLETPSLLL | APC | 4229 |
| MLL2 L8H | ALSPVIPHI | APC | 4230 |
| SMARCD3 H8Y | KLFEFLVYGV | APC | 4231 |
| GNL3L R4C | NLNCCSVPV | APC | 4232 |
| USP28 C5F | LIIPFIHLI | APC | 4233 |
| MRM1 T6P | LLFGMPPCL | APC | 4234 |
| SNX24 P6L | KLSHQLVLL | APC | 4235 |
| PGM5 H5Y | AVGSYVYSV | APC | 4236 |
| SEC24A P5L | FLYNLLTRV | APC | 4237 |
| AKAP13 Q8K | KLMNIQQKL | APC | 4238 |
| PABPC1 R5Q | MLGEQLFPL | APC | 4239 |
| MAGE-A3 112-120 | KVAELVHFL | PE | 4240 |
| MAGE-A12 112-120 | KMAELVHFL | APC | 4241 |
| MAGE-A2 112-120 | KMVELVHFL | APC | 4242 |
| MAGE-A6 112-120 | KVAKLVHFL | APC | 4243 |

Experiment 5

| | | Tetramer | |
|---|---|---|---|
| Peptide Name | Sequence | Fluorescence | SEQ ID |
| A2ML1-YLD_K7R | YLDELIRNT | PE | 4244 |
| AGFG2-FLQ_S4S | FLQFRGNEV | PE | 4245 |
| AGXT2 L2-ILT_M5I | ILTDIEEKV | PE | 4246 |
| AHNAK-SMP_S1F | FMPDFDLHL | PE | 4247 |
| AKAP13-KLM_Q8K | KLMNIQQKL | PE | 4248 |
| APBB2-GML_L3F | GMFPVDKPV | PE | 4249 |
| APBB2-VQY_L7F | VQYLGMFPV | PE | 4250 |
| APCDD1L-RLP_R1W | WLPHVEYEL | PE | 4251 |
| ATP6AP1-KLG_G3W | KLWASPLHV | PE | 4252 |
| BAIAP3-ILN_V61 | ILNVDIFTL | PE | 4253 |
| BCL9L-FVY_T6I | FVYVFITHL | PE | 4254 |
| BTBD1-FML_LI | FMLLTQARI | PE | 4255 |
| C15orf32-MLS_G9R | MLSILALVRV | PE | 4256 |
| C17orf75-ALS_V7A | ALSYTPAEV | PE | 4257 |
| C1S-10_N1H | HLMDGDLGLI | PE | 4258 |
| C1S-9_N1H | HLMDGDLGL | PE | 4259 |
| C3orf58-LMV_L4P | LMVPHSPSL | PE | 4260 |
| CAMK1D-KLF_K8N | KLFEQILNA | PE | 4261 |
| CCM2-YML_R6H | YMLTLHTKL | PE | 4262 |
| CD47-GLT_V6F | GLTSFFIAI | PE | 4263 |
| CDC37L1-FLS_P6L | FLSDHLYLV | PE | 4264 |
| CELSR1-YLF_F3L | YLLAIFSGL | PE | 4265 |
| CHD8-KLN_P7A | KLNTITAVV | PE | 4266 |
| CHST13-VLV_V1M | MLVDDAHGL | PE | 4267 |
| CHST14-MLM_F4L | MLMLAVIVA | PE | 4268 |
| CLCN4-LLA_G8V | LLAGTLAVV | PE | 4269 |
| CNKSR1-SLA_A9V | SLAPLSPRV | PE | 4270 |
| COL18A1-VLL_S8F | VLLGVKLFGV | PE | 4271 |
| DCHS1-TLF_I5M | TLFTMVGTV | PE | 4272 |
| DHX33-LLA_K5T | LLAMTVPNV | PE | 4273 |
| DHX33-LLA_M4I | LLAIKVPNV | PE | 4274 |
| DNAH8-FMT_G7D | FMTKINDLEV | PE | 4275 |
| DOCK7-FLN_M9L | FLNDLLSVL | PE | 4276 |
| DOLPP1-GLM_A4V | GLMVIAWFI | PE | 4277 |
| DRAM1-FII_I3F | FIFSYVVAV | PE | 4278 |
| ERBB2-ALI_H8Y | ALIHHNTYL | PE | 4279 |
| EXOC3L4-ILL_V9I | ILLDWAANI | PE | 4280 |
| FAM47B-ALF_A1S | SLFSELSPV | PE | 4281 |
| FBXL4-SLL_L2V | SVLEYYTEL | PE | 4282 |
| FLNA-HIA_P6L | HIAKSLFEV | PE | 4283 |
| FNDC3B-VVL_L3M | VVMSWAPPV | PE | 4284 |
| GABRG3-TAM_L5I | TAMDIFVTV | PE | 4285 |
| GABRG3-YVT_L7I | YVTAMDIFV | PE | 4286 |
| GALC-YVV_V3L | YVLTWIVGA | PE | 4287 |
| GANAB-ALY_S5F | ALYGFVPVL | PE | 4288 |
| GCN1L1-10_L6P | ALLETPSLLL | PE | 4289 |
| GCN1L1-9_L6P | ALLETPSLL | PE | 4290 |
| GLRA1-LIF_F6L | LIFNMLYWI | PE | 4291 |
| GOLGA3-SLD_P4L | SLDLTTSPV | PE | 4292 |
| GPR137B-KMS_S3P | KMPLANIYL | PE | 4293 |
| GPR174-FSF_P4S | FSFSLDFLV | PE | 4294 |
| GSTA4-FLQ_E4K | FLQKYTVKL | PE | 4295 |
| HAUS3-ILN_T7A | ILNAMIAKI | PE | 4296 |
| HBZ-KLS_A7T | KLSELHTYI | PE | 4297 |
| HERC1-SLL_PS | SLLLLSVSV | PE | 4298 |
| HLA-DRB5-YMA_KE | YMAELTVTL | PE | 4299 |
| HOXC9-YMY_G4D | YMYDSPGEL | PE | 4300 |
| HTR1F-10_V1M | MMPFSIVYIV | PE | 4301 |
| HTR1F-9_V1M | MMPFSIVYI | PE | 4302 |
| HTR1F-LVM_V2M | LMMPFSIVYI | PE | 4303 |
| IGF1-TMS_S4F | TMSFSHLFYL | PE | 4304 |
| IL17RA-FIT_TM | FIMGISILL | PE | 4305 |
| INTS1-VLL_L3F | VLFHRAFLV | PE | 4306 |
| IPO9-FSS_E4D | FSSDVLNLV | PE | 4307 |
| ITIH6-RLG_G3V | RLVPYLEFL | PE | 4308 |
| KAT6A-KLS_MK | KLSREIKPV | PE | 4309 |
| KCNB2-LLA_P6T | LLAILTYYV | PE | 4310 |
| KCNC3-FLP_A7V | FLPDLNVNA | PE | 4311 |
| KIF20B-YTS_S6L | YTSEILSPI | PE | 4312 |
| LCP1-NLF_PL | NLFNRYLAL | PE | 4313 |
| MAR11-10_F1L | LLIASVTWLL | PE | 4314 |
| MAR11-9_F1L | LLIASVTWL | PE | 4315 |
| ME1-FLD_A8G | FLDEFMEGV | PE | 4316 |
| MLL2-ALS_L8H | ALSPVIPHI | PE | 4317 |
| MPV17-YLW A5P | YLWPPVQLA | PE | 4318 |
| MRGPRF-RLW_R1W | WLWEPLRVV | PE | 4319 |
| MRM1-10_T6P | LLFGMPPCLL | PE | 4320 |
| MRM1-9_T6P | LLFGMPPCL | PE | 4321 |
| MYH4-GLD_D3N | GLNETIAKL | PE | 4322 |
| MYPN-RVI_R1L | LVIGMPPPV | PE | 4323 |
| NBPF24-LLD_E6G | LLDEKGPEV | PE | 4324 |
| NOS1-FID_D3Y | FIYQYYSSI | PE | 4325 |
| NSDHL-ILT_A9V | ILTGLNYEV | PE | 4326 |
| OASL-ILD_DN | ILNPADPTL | PE | 4327 |
| OR10A3-ILI_V6F | ILIVMFPFL | PE | 4328 |
| OR14C36-FML_V6L | FMLYLLTLM | PE | 4329 |
| OR1G1-FLF_T8M | FLFMYLVMV | PE | 4330 |
| OR2T1-FLN_F5L | FLNVLFPLL | PE | 4331 |
| OR5K2-YIF_GE | YIFLENLAL | PE | 4332 |
| OR5M3-KMV_T8N | KMVAVFYNT | PE | 4333 |
| OR6F1-VLN_T8M | VLNPFIYML | PE | 4334 |
| OR8B8-YVN_V2L | YLNELVVFV | PE | 4335 |
| OR8D4-10_G3E | FLEIYTVTVV | PE | 4336 |
| OR8D4-9_G3E | FLEIYTVTV | PE | 4337 |
| OR9Q2-FLF_S8F | FLFTFFAFI | PE | 4338 |
| OR9Q2-SID_S1F | FIDCYLLAI | PE | 4339 |
| OVOL1-SLL_L9V | SLLQGSPHV | PE | 4340 |
| PABPC1-MLG_R5Q | MLGEQLFPL | PE | 4341 |
| PCDHB3-FLF_SL | FLFLVLLFV | PE | 4342 |
| PELP1-LVL_L3F | LVFPLVMGV | PE | 4343 |
| PELP1-RLH_L7F | RLHDLVFPL | PE | 4344 |
| PGM5-AVG_H5Y | AVGSYVYSV | PE | 4345 |
| PHKA2-LLS_SF | LLSIIFFPA | PE | 4346 |
| PIGN-FLT_P7H | FLTVFSHFM | PE | 4347 |
| PLXNB1-VLF_V1L | LLFAAFSSA | PE | 4348 |
| PRSS16-LLL_L1Q | QLLVSLWGL | PE | 4349 |
| PTCHD4-HQL_G5V | HQLGVVVEV | PE | 4350 |
| PXDNL-SIL_S1F | FILDAVQRV | PE | 4351 |
| REV3L-KLS_R6H | KLSEYHNSL | PE | 4352 |
| RRP1B-LLA_L7F | LLADQNFKFI | PE | 4353 |
| RYR3-VLN_E6K | VLNYFKPYL | PE | 4354 |
| SCN3A-ALV_P7S | ALVGAISSI | PE | 4355 |
| SEC24A-FLY_P5L | FLYNLLTRV | PE | 4356 |
| SH3RF2-HMV MI | HIVEISTPV | PE | 4357 |
| SHROOM2-KLL_D6V | KLLAGVEIV | PE | 4358 |
| SLC15A2-ILG_G4E | ILGEQVVHTV | PE | 4359 |
| SLC16A7-AMA_P6L | AMAGSLVFL | PE | 4360 |
| SLC1A2-YMS_S3P | YMPTTIIAA | PE | 4361 |
| SLC2A3-ILV_L9M | ILVAQIFGM | PE | 4362 |
| SLC2A4-ILI_A4T | ILITQVLGL | PE | 4363 |
| SLC38A1-RIW_W3L | RILAALFLGL | PE | 4364 |
| SLC39A4-LLG_G4S | LLGSVVTVLL | PE | 4365 |
| SMARCD3-KLF_H8Y | KLFEFLVYGV | PE | 4366 |
| SMOX-KLA_KN | KLANPLPYT | PE | 4367 |
| SNX24-KLS_P6L | KLSHQLVLL | PE | 4368 |
| SPOPN1471-FLL_N7I | FLLDEAIGL | PE | 4369 |
| SREBF1-YLQ_L6M | YLQDSMATT | PE | 4370 |
| SSPN-10_S9F | FLMASISSFL | PE | 4371 |
| SSPN-9_S9F | FLMASISSF | PE | 4372 |
| SSPN-LMA_S8F | LMASISSFL | PE | 4373 |
| ST6GALNAC2- | LLFALHFSA | PE | 4374 |
| LLF_Y6H | | | |
| STOX1-RLM_M31 | RLIKHYPGI | PE | 4375 |
| TAS1R2-FMS_A4S | FMSSYSGVL | PE | 4376 |
| TBX3-GMG_T8M | GMGPLLAMV | PE | 4377 |
| TEAD1-SVL_L9F | SVLENFTIFL | PE | 4378 |
| TEAD1-VLE_L8F | VLENFTIFLV | PE | 4379 |
| TEX2-FLM_K8N | FLMTLETNM | PE | 4380 |
| TMEM127-VTF_L9V | VTFAVSFYVV | PE | 4381 |
| TMEM195-ALS_S3L | ALLQVTLLL | PE | 4382 |
| TP73-YTP_P3S | YTSEHAASV | PE | 4383 |
| TPP2-SLA_WL | SLAETFLET | PE | 4384 |
| TRIM16-RMA_R1T | TMAAISNTV | PE | 4385 |
| TRIM58-VLA_V1F | FLASPSVPL | PE | 4386 |
| TRIM58-YMV_V3F | YMFLASPSV | PE | 4387 |
| TRPC1-MLL_Q5H | MLLKHDVSL | PE | 4388 |
| TRPV3-LLL_A8V | LLLNMLIVL | PE | 4389 |
| TRPV4-FMI_A6T | FMIGYTSAL | PE | 4390 |
| TRPV4-YLL_A9T | YLLFMIGYT | PE | 4391 |
| TTLL12-KLP_N7D | KLPLDIDPV | PE | 4392 |
| UNC13A-SQL_S1F | FQLNQSFEI | PE | 4393 |
| USP28-LII_C5F | LIIPFIHLI | PE | 4394 |
| VN1R2-LML_L3F | LMFWASSSI | PE | 4395 |
| VN1R5-MII_S7Y | MIISHLYLI | PE | 4396 |
| WDR46-FLT T3I | FLIYLDVSV | PE | 4397 |
| ZDHHC17-LLL_T4I | LLLIFNVSV | PE | 4398 |
| ZDHHC7-SLL_P7L | SLLWMNLFV | PE | 4399 |
| ZFP9O-FTQ_EK | FTQEKVVYHV | PE | 4400 |
| ZNF827-NLF_S4I | NLFIQDISV | PE | 4401 |
| HCV-KLV (PE) | KLVALGINAV | PE | 4402 |
| HCV-KLV (APC) | KLVALGINAV | APC | 4403 |
| EMPTY | | APC | 4404 |
| EMPTY | | PE | 4405 |

### Example 3

### 3′ End Sequencing of Highly Multiplexed Single Cell RNA-Seq Libraries

3′ end sequencing of RNA transcripts is a robust and popular method for analyzing transcriptome expression within a population of cells as well as single cells, though multiplexed single cell transcriptome sequencing has proved challenging. Populations of seemingly homogenous populations of cells are known to have a great deal of heterogeneity in gene expression, confounding bulk transcriptome sequencing. Current methods of single cell sequencing attempt to address that problem, though these methods have a relatively low throughput and are extremely costly. 3′ enrichment is challenging in the currently available methods as both 3′ and 5′ ends have the same adaptor sequence. The ability to highly multiplex is also limited with the primers available.

To address these challenges, a new method of 3′ end sequencing of RNA-seq libraries was developed for highly multiplexed samples. cDNA amplification was performed essentially as in the Smart-Seq2 protocol (Picelli et al., 2013) with several important modifications. A unique cell barcode is included in the reverse transcription (RT) primer, and a restriction digest (SalI) site is included in the template switching oligo (TSO) (Table 1) RT primers with unique cell barcodes were individually dispensed into each well of a 384-well PCR plate.

The workflow for the 3′ end sequencing is shown in FIG. 23A. Briefly, single cells are sorted into individual wells by indexed FACS sorting, and lysed. cDNA amplification is performed essentially as in the Smart-Seq2 protocol, but with the primers listed above (Picelli et al., 2013). After cDNA amplification, multiple single cell PCR products are pooled, each of which already has unique cell barcode at the 3′ end. After purification, PCR products are digested by restriction enzyme incubation. Libraries are then prepared from the digested products using a modified Nextera XT protocol in which custom primers designed to enrich 3′ end are used.

The libraries were then sequenced on an Illumina® NextSeq to a depth of 500,000 reads. The data was then analyzed using custom scripts. It was found that inclusion of restriction enzyme digestion improved recovery of 3′ end sequences significantly over other 3′ selection methods, recovering between 80 and 89% of 3′ end sequences that have cell barcode information (Table 11). Enrichment was measured as the number of reads with all of the correct barcode sequences in read1 divided by the total raw reads.

TABLE 11
3′ end enrichment

| | Percentage of | Genome Mapping |
|---|---|---|
| Method | 3′ end enrichment | percentage |
| w/o restriction enzyme digestion* | 12.96% | 9.64% |
| w/restriction enzyme digestion | 80.02% | 37.39% |
| w/restriction enzyme digestion and | 89.06% | 43.53% |
| gel purification | | |

*customized nextera PCR primer with four base pairs that are only complementary to the RT primer and mismatches to TSO

In addition to significantly enriching the 3′ ends of the transcripts, by using 384-well PCR plate the reaction volume is significantly decreased, while the ability to multiplex is significantly increased, compared to the original Smart-seq2 method.

Next, an ERCC spike-in was performed to validate this protocol 5 nl of 1:40,000 diluted ERCC were added into each well of sorted single cells. The data from the ERCC spike-in was then compared to published data. The method of 3′ end sequencing presented herein was shown to have a similar ERCC detection efficiency to published scRNA-seq data, demonstrating the reliability of this method (FIG. 23B). The correlation between the 3′ end-seq method presented herein and the original Smart-seq2 method was also found to be high (r²=0.924) when comparing normalized reads per million (RPM) (FIG. 23C).

Cross contamination during the 3′ end sequencing protocol was examined next. Human and Mouse cDNA were prepared separately according to the 3′ end sequencing method presented above, but with different cellular barcodes. The cDNAs were then mixed and sequenced as above. Sequencing data were mapped to human and mouse transcriptome respectively using Kallisto. The transcript mapping percentages were compared and it was found that there was a very low cross-contamination rate after sample pooling (FIG. 23D).

The methods disclosed herein allow for highly multiplexed RNA sequencing and will be increasingly valuable as scientists seek to understand and compare increasing numbers of single cells. As shown, these methods provide robust enhancement of 3′ ends of RNA for transcriptome profiling, and excellent multiplexing capabilities. 3′ end sequencing will also add another dimension to T cell profiling and can be incorporated into the TetTCR-seq workflow to assess the transcriptome of the targeted cells. These methods could be extended to methods with even greater multiplexing such as droplet and microwell based single cell RNA-seq or targeted amplification and sequencing selected genes, and digital PCR and sequencing methods.

### Example 4

Studies were performed to examine T cell antigen binding and their associated activation and phenotype in human CD8 cells.

In brief, each peptide barcode was individually in vitro transcribed/translated (IVTT) to generate corresponding peptide, which was later loaded onto MHC molecules. Then pMHC tetramer was tagged with its corresponding peptide barcode bearing a 3′ polyA overhang (FIG. 24). This enables the tetramer barcodes to be captured by BD Rhapsody beads and can be processed together with mRNA through BD Rhapsody. Similar as BD Rhapsody bioinformatic pipeline, peptide barcode sequencing reads from putative cells were extracted and mapped to peptide barcode reference. Only reads that are exact map were retained. The number of unique molecular identifiers (MIDs) was counted for each peptide barcode among individual cells.

Two passes were implemented to call tetramer specificity for each cell, in order to increase the precision. In the first pass, MID negative thresholds were then determined for foreign- and self-peptides respectively. Distribution of MID count aggregation was modeled through bimodal distribution. Specificities of putative tetramer positive cell were identified independently by inflection point of MID counts among all peptides. In the second pass, paired TCRa/b were further integrated with tetramer specificity called from first pass to correct for false positives and false negatives. It was assumed that T cells bearing same paired TCR α/β have the same tetramer specificity. Among T cells having multiple specificities (or tetramer negatives) associated with same TCR, their specificity was correct as the dominant tetramer specificity.

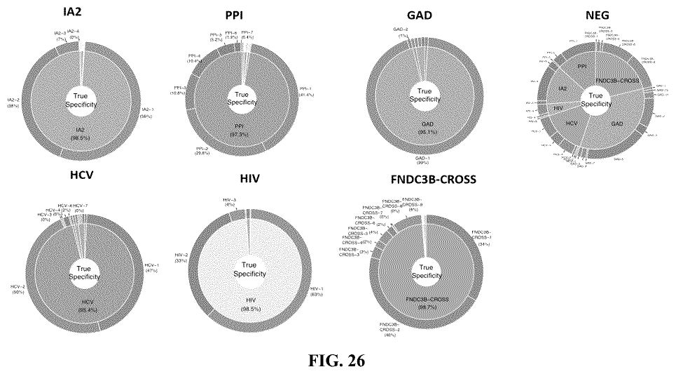

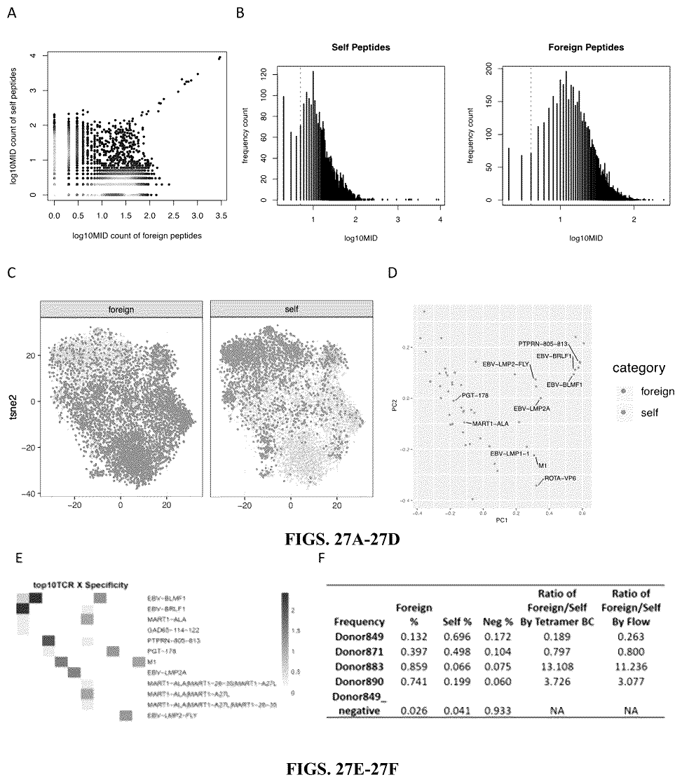

TetTCR-SeqHD was first applied on a mixture of polyclonal T cell populations, including IA2, PPI, GAD, HCV, HIV, FNDC3B-derived antigen specific clones (FIG. 25A-B). Over 80% of cells have paired TCR α/β (FIG. 25C). The peptide molecular counts were examined and three populations were easily observed, including self-antigen specific cells, foreign-antigen specific cells and a cross-reactive population (FIG. 25D). The TCR sequence of each cell represents its true tetramer specificity. After 1^{st} pass of tetramer specificity call, the precision of calling the correct tetramer specificity was found to be over 95% for all the clones with a FDR less than 5% (FIG. 26). Further analysis of the TCR sequences of each antigen specificity population recaptures the original distribution of TCR clonality (FIG. 27), further demonstrating the robustness of TetTCR-SeqHD to reveal the true identity of T cell antigen specificity.

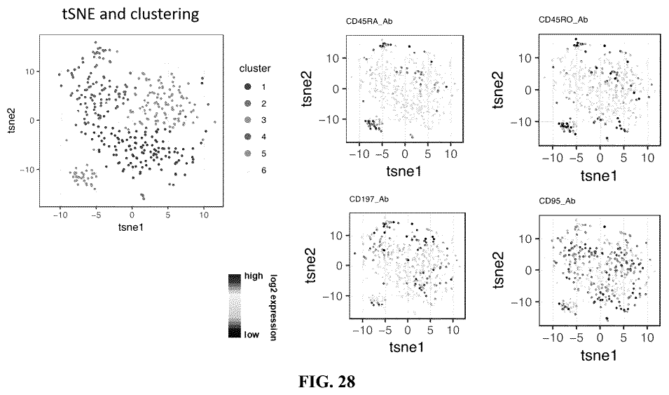

After validation of TetTCR-SeqHD using T cell clones, this technology was further applied to study differences of foreign- and self-specific T cells from human primary CD8 T cells. A total of 80 self-specific peptides were curated through the IEDB database, as well as 33 influenza-, HIV-, EBV-, CVB, Rotaviruse- and HCV-derived peptides. Enriched CD8 T cells were processed from four different donors. The peptide molecular counts were evaluated with density plot and two populations were easily observed, self-antigen specific population and foreign-antigen specific population (FIG. 28A). Due to the low similarity of self- and foreign peptides, a significant cross-reactive population was not observed. Further, by applying self- and foreign peptide molecular count distribution, the negative threshold was bioinformatically inferred to call positive tetramer binding event for each experiment (FIG. 28B). The gene expression profiles for different antigen specificities were compared and it was found that self-antigen specific T cells are phenotypically different compared with foreign-antigen specific T cells (FIG. 29C-D). Moreover, TCR sequences were used to further prove the accuracy of antigen-specificity identification using pMHC DNA barcodes (FIG. 28E). The top 10 TCRs show minimal noisy antigen-specificity identification other than the true identity. Meanwhile, the ratio between self- and foreign-antigen specific T cells identified by pMHC DNA barcodes resembles the ratio from flow cytometry data for all the donors (FIG. 28F).

Last, it was also demonstrated that proteogenomics profile can be investigated in combination with TetTCR-SeqHD, using DNA-labeled antibody sequencing, such as CITE-seq or REAP-seq or the commercially available DNA-labeled antibodies, such as BD Ab-seq products or Biolegend TotalSeq (FIG. 29) (Stoeckius et al., 2017). Using DNA-labeled antibody, primary CD8 T cells can be easily separated into naïve, central memory, effector memory, effector CD8 T cells using canonical antibodies such as CCR7, CD45RA, CD45RO and CD95.

The method disclosed here in can be applied to study the phenotypic profiles of antigen specific T cells in various diseases, including but not limited to autoimmune diseases, such as type 1 diabetes, multiple sclerosis, Rheumatoid arthritis, Lupus, Celiac disesase and so on, various cancers, and infectious diseases.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bentzen, A. K. et al. Nat Biotech 34, 1037-1045 (2016).
Bernard et al, Anal. Biochem., 273: 221-228 (1999).
Birnbaum, Michael E. et al. Cell 157, 1073-1087 (2014).
Bullock, T. N. J. et al. The Journal of Immunology 167, 5824-5831 (2001).
Cameron, B. J. et al. Science Translational Medicine 5, 197ra103-197ra103 (2013).
Carreno, B. M. et al. Science 348, 803-808 (2015).
Cohen, C. J. et al. The Journal of Clinical Investigation 125, 3981-3991 (2015).
Dietrich, P.-Y. et al. The Journal of Immunology 170, 5103-5109 (2003).
Dudley, M. E. et al. Science (New York, N.Y.) 298, 850-854 (2002).
Fu, G. K. et al. Analytical Chemistry 86, 2867-2870 (2014).
Glanville, J. et al. Nature 547, 94-98 (2017).
Lang, H. L. et al. Nature immunology 3, 940-943 (2002).
Luimstra et al., Journal of Experimental Medicine, 1-11 (2018).
Macoscko et al., Cell, 161(5): 1202-1214 (2015).
Mongkolsapaya, J. et al. Nat Med 9, 921-927 (2003).
Newell, E. W. & Davis, M. M. Nature biotechnology 32, 149-157 (2014).
Newell, E. W. et al. Nat Biotech 31, 623-629 (2013).
Peterson, V. M. et al. Nature Biotechnology 35, 936 (2017).
Picelli et al., Nature Methods, 10:1096-1098 (2013).
Picelli et al., Nature Protocols, 9(1): 171-181 (2014).
Rajasagi, M. et al. Blood 124, 453 (2014).
Ramskold et al., Nature Biotechnology, 30, 777-782 (2012).
Rodenko, B. et al. Nat. Protocols 1, 1120-1132 (2006).
Stoeckius et al., Nature Methods, 14, 865-868 (2017).
Stronen, E. et al. Science (2016).
Yu, W. et al. Immunity 42, 929-941 (2015).
Zhang, S.-Q. et al. Science Translational Medicine 8, 341ra377-341ra377 (2016).

What is claimed is:

1. A method for producing a DNA-barcoded peptide multimer comprising:
(a) performing in vitro transcription/translation (IVTT) on a peptide-encoding oligonucleotide to obtain a library of peptides;
(b) contacting the library of peptides with biotinylated MHC monomers comprising UV-cleavable peptides;
(c) applying UV light to exchange the UV-cleavable peptides on the MHC monomers with the library peptides to form peptide MHC monomers (pMHCs);
(d) combining the pMHCs with a multimer backbone comprising streptavidin, wherein streptavidin is linked to an oligonucleotide comprising the peptide-encoding oligonucleotide via a DNA handle comprising a molecular identifier (MID) to produce a DNA-barcoded peptide multimer.

2. The method of claim 1, wherein the multimer backbone comprising streptavidin is prepared by:
a. linking a DNA handle comprising the molecular identifier (MID) to streptavidin in a single batch; and
b. linking the oligonucleotide comprising the peptide-encoding oligonucleotide to the DNA handle in individual reactions, prior to combining the pMHCs with the multimer backbone.

3. The method of claim 2, wherein each DNA-barcoded pMHC multimer has a similar DNA handle:multimer backbone ratio.

4. The method of claim 2, wherein the DNA handle is linked to streptavidin comprising a fluorescent tag.

5. The method of claim 4, wherein the streptavidin is R-phycoerythrin-streptavidin or Allophycocyanin-streptavidin.

6. A method of generating a library of DNA-barcoded pMHC multimers comprising performing the method of any one of claim 1, with a plurality of peptide-encoding DNA oligonucleotides.

7. A DNA-barcoded peptide multimer library produced by the method of claim 1.

8. A method for determining the specificity of T cell receptors (TCRs), the method comprising:
(a) staining a plurality of T cells with a library of DNA-barcoded peptide multimers of claim 7, to generate peptide multimer-bound T cells;
(b) sorting the peptide multimer-bound T cells by separating the peptide multimer-bound T cells from unbound T cells;
(c) sequencing the molecular identifier (MID) of each peptide multimer and a cDNA encoding variable regions of TCR sequences of the T cell bound to said peptide multimer; and
(d) determining the copy number of DNA-barcoded peptide multimers bound to the corresponding T cell to determine TCR specificity, and wherein the copy number is determined by counting the number of copies of each MID.

9. The method of claim 8, wherein the sorting comprises performing flow cytometry.

10. The method of claim 8, wherein the sorting comprises separating single DNA-barcoded peptide multimer-bound T cells into separate reaction containers.

11. The method of claim 10, wherein the reaction container is multi-well plate.

12. The method of claim 8, wherein sequencing comprises preparing DNA-sequencing libraries, the preparing comprising at least one amplification step wherein a primer pair is used to amplify the MID and a different set of primer pairs is used to amplify variable regions of TCRα and TCRβ, sequences of the T cells.

13. A method for linking precursor T cells to their specific antigens comprising:
(a) staining a plurality of T cells with a library of DNA-barcoded peptide multimers of claim 7, to generate peptide multimer-bound T cells;
(b) enriching for peptide multimer-bound precursor T cells;
(c) sorting the peptide multimer-bound precursor T cells by separating the peptide multimer-bound T cells from unbound T cells;
(d) calculating the frequency of peptide multimer-bound precursor T cells;
(e) sequencing (i) the MID of each peptide multimer and (ii) a cDNA encoding variable regions of TCR sequences of the precursor T cells bound to said peptide multimer;
(f) determining the copy number of each DNA-barcoded peptide multimer bound to the corresponding precursor T cell to determine TCR specificity, wherein the copy number is determining by counting the number of copies of each MID.

14. The method of claim 13, wherein the sorting comprises performing flow cytometry.

15. The method of claim 13, wherein the sorting comprises separating single DNA-barcoded peptide multimer-bound T cells into separate reaction containers.

16. The method of claim 15, wherein the reaction container is multi-well plate.

17. The method of any one of claim 13, wherein sequencing comprises preparing DNA-sequencing libraries, the preparing comprising at least one amplification step wherein a primer pair is used to amplify the MID and a different set of primer pairs is used to amplify variable regions of TCRα and TCRβ sequences of the T cells.
